SEQUENCE LISTING (283 sequences)

SEQ ID NO: 1 - DNA, 3871 bp, Homo sapiens
tttccttctccctccccttttcccttccttcgtcccttccttccttcctttcgccgggcgcgatggagccggggcgccggggggccgcggcgctgctagcgctgctgtgcgtggcctgcgcgctgcgcgccgggcgcgcccaatacgaacgctacagcttccgcagcttcccacgggacgagctgatgccgctcgagtcggcctaccggcacgcgctggacaagtacagcggcgagcactgggccgagagcstkggctacctggagatcagcctgcggctgcaccgcttgctgcgcgacagcgaggccttctgccaccgcaactgcagcgccgcgccgcagcccgagcccgccgccggcctcgccagctatcccgagctgcgcctcttcgggggcctgctgcgccgcgcgcactgcctcaagcgctgcaagcagggcctgccagccttccgccagtcccagcccagccgcgaggtgctggcggacttccagcgccgcgagccctacaagttcctgcagttcgcttacttcaaggcaaataatctccccaaagccatcgccgctgctcacacctttctactgaagcatcctgatgacgaaatgatgaagaggaacatggcatattataagagcctgcctggtgccgaggactacattaaagacctggaaaccaagtcatatgaaagcctgttcatccgagcagtgcgggcatacaacggtgagaactggagaacatccatcacagacatggagctggcccttcccgacttcttcaaagccttttacgagtgtctcgcagcctgcgagggttccagggagatcaaggacttcaaggatttctacctttccatagcagatcattatgtagaagttctggaatgcaaaatacagtgtgaagagaacctcaccccagttataggaggctatccggttgagaaatttgtggctaccatgtatcattacttgcagtttgcctattataagttgaacgacctgaagaatgcagccccctgtgcagtcagctatctgctctttgatcagaatgacaaggtcatgcagcagaacctggtgtattaccagtaccacagggacacttggggcctctcggatgagcacttccagcccagacctgaagcagttcagttctttaatgtgaccacactccagaaggagctgtatgactttgctaaggaaaatataatggatgatgatgagggagaagttgtggaatatgtggatgacctcttggaactggaggagaccagctagcccacagcaaccaaagagacttcctcttggcgttcaggaaacacagattctttgtccttttcccaacagcccaggctgttgatacctcagagccttctctttactctccaaagtgaaagggaagcccccgtctctctaactgcatgtcatcaggggtgagcctgcctttcctatcttcacacctgccacctcatgttcacacctatctttctcaccttttttttgagatggagtctcgctctcttgcccaggctggagtgcaatggcacgttctcagctcactgcaacctccgcctcttgggttcaagcaattctgctgcatcagcctcccgagtacctgggattacaggcatgtgccaccacgcccggctaattttgtatttttagtagagacggggttttgccatgttggccaggctggtctcgaactcttgacttcagatgatccatctgccttggcctcccacagtgctgggattacaggcgtgagccaccatgcccggcctctttctcacctttacacctgtcttcttatcctcacatctgttttcacaccttcatccctgtcttcctcatgttcacacttgtcttccccatgttcatagctgcctttcttaccattttggtttgaagggcagtcttctctggcttgtttttttgtttttcccagaaaatcagtattattttttaaataagaaaaacattcctagaagatgataattgtgaaaacctcctttggcttatttgcttttccagattttagtctcctttctccccatccgggaaagatggtggaagacataggctaaatttctccagcctcacaatggtcttcacttggtctgacttgtaccaattctagcacccactgaaaaacaagttgagtagagagtgtagagtgcagaaatgtggcttttgccccactttgcatctccaaaattacaacggttggccgatcccatttgaggacaatgcttagttataagtctccgagttggaaaaggaagaaagccagagctgtctagtttcattcattctttcagtaaatatttattgagtacctactgtgtgctaggcattgacctgggaactagagatacttcacagaataacagggaaagttccctgtgctcatggagcttacattctacagggagaaagagatagccaatacataggaataaatatatacaaggtatcatgtagtgataattgctgtggagaaaaataaagcaggggagggagtaagaaatcctggagatgaggctgcagttttaaatggggcctcactgggaatgtgacgttgagcagagacgttagggaagtggatcctggacaaggcattccaggcagaggaacaagatgtgcactgccccaaagtgagaacttgctctacgtggtcaggaaagagcagggagaccaagcagagtcgtgggcaggggtagaatggaaggagaggcggctggggaggacaggtggtggagggccttggcttctgctaagtgagatgggaaccactggagggtttgaacagaggagtgccttgattgatttatattttgcaagggtcattctagctgcaatattgtgaaaaactttagtggacaagggcagaaggaagagggaagacctgttaggaagctactgcaaggttccaggcttgggcctgggccacagcaacagcagtggtcaaatatctagatttattttgaaaagagccaataggatttgctgagagtttgaatgtggagtgtaagagaaggaagagttaatgatgacattaaggtttttggcctgaatagcaggaaagatggagttaccagttactgaaatagggaaggatgggctgggtaagtaaggaatttggtgcaaagcaggctgtctgtggttggaatgggaggttctggctgcaaatcaaagtggagattctctcaggtcaggtctgcagcagagctcgagacagggatctgaatgcacttggtttattgttgggggtgctctcagaaggaacctgtgaaagcctttatcagtcatttattggctgtgagaagttctctgggagtgtgggtacatttgaaggcaagtgacttcagttgagggcaagtctctggaaaagaggctgtaggcatctggcagctaccatgcgtggtagtgtgttgggggtgggggtcctgggcactggctgtgtgaagggatctggcagggcaccacagcgccccctactgaaccatcagcatgtcagtggcatttaaagccatgcagctggaggggccactgagattgtctctgagtattactgagaagcaacagaaaagagccatggatggagcccttgggctctctgggaaatgggaaatcagccaaaggactgagaaggagttaccttaaggtcagagaaaaccaagagagtgtggtgttctggaagctgagctttctttattcaacctcattcccttctccaaataagccacttgtgtagttgggcccctccagggttgaaggcaagaggagaaaggcacagcgtttgggaaacaagacttttcctgcaatagcctgggaaggaataaaaggatagagtgttaaaataaaaaaaaaaaaaaaaa

SEQ ID NO: 2 - protein, 401 aa, Homo sapiens
  UNSURE (64)
MEPGRRGAAALLALLCVACALRAGRAQYERYSFRSFPRDELMPLESAYRHALDKYSGEHWAESXGYLEISLRLHRLLRDSEAFCHRNCSAAPQPEPAAGLASYPELRLFGGLLRRAHCLKRCKQGLPAFRQSQPSREVLADFQRREPYKFLQFAYFKANNLPKAIAAAHTFLLKHPDDEMMKRNMAYYKSLPGAEDYIKDLETKSYESLFIRAVRAYNGENWRTSITDMELALPDFFKAFYECLAACEGSREIKDFKDFYLSIADHYVEVLECKIQCEENLTPVIGGYPVEKFVATMYHYLQFAYYKLNDLKNAAPCAVSYLLFDQNDKVMQQNLVYYQYHRDTWGLSDEHFQPRPEAVQFFNVTTLQKELYDFAKENIMDDDEGEVVEYVDDLLELEETS

SEQ ID NO: 3 - DNA, 3637 bp, Homo sapiens
  unsure (1582)
tttttttttttttttttttaagaagaaggtccaaatcaataggtcttttattgcatcatttaaatatcacaagtaggtcttaagtgtcatctggcatcttctttctgtagccaggtaactcttagatcttattcatcagcctgctgaacagttcctttttcagagacatagataccatccaaaaatttcctgatatccttgtttttaactgttgtggcttgctgaatcaaagccgctgaatttgaaacaagctcaatgtcatcccgattgagtaccagctccccactgccctgagggcgggccggcctgcggcggagggaaaaaggaagaggagaaggaaattgtcccgaatccctgcagtctttctgtaggttgcggcacaacgccaggcaaaagaagaggaaggaatttaatcctaatcggtggaggtcgatttgagggtctgctgtagcaggtggctccgcttgaagcgagggaggaagtttcctccgatcagtagagattggaaagattgttgggagtggcacaccactagggaaaagaagaaggggcgaactgcttgtcttgaggaggtcaacccccagaatcagctcttgtggccttgaagtggctgaagacgatcaccctccacaggcttgagcccagtcccacagccttcctcccccagcctgagtgactactctattccttggtccctgctattgtcggggacgattgcatgggctacgccaggaaagtaggctgggtgaccgcaggcctggtgattggggctggcgcctgctattgcatttatagactgactaggggaagaaaacagaacaaggaaaaaatggctgagggtggatctggggatgtggatgatgctggggactgttctggggccaggtataatgactggtctgatgatgatgatgacagcaatgagagcaagagtatagtatggtacccaccttgggctcggattgggactgaagctggaaccagaactagggccagggcaagggccagggctacccgggcacgtctggctgtccagaaacgggcttcccccaattcagatgataccgttttgtcccctcaagagctacaaaaggttctttgcttggttgagatgtctgaaaagccttatattcttgaagcagctttaattgctctgggtaacaatgctgcttatgcatttaacagagatattattcgtgatctgggtggtctcccaattgtcgcaaagattctcaatactcgggatcccatagttaaggaaaaggctttaattgtcctgaataacttgagtgtgaatgctgaaaatcagcgcaggcttaaagtatacatgaatcaagtgtgtgatgacacaatcacttctcgcttgaactcatctgtgcagcttgctggactgagattgcttacaaatatgactgttactaatgagtatcagcacatgcttgctaattccatttctgacttttttcgtttattttcagcgggaaatgaagaaaccaaacttcaggttctgaaactccttttgaatttggctgaaaatccagccatgactagggaactgctcagggcccaagtaccatcttcnctgggctccctctttaataagaaggagaacaaagaagttattcttaaacttctggtcatatttgagaacataaatgataatttcaaatgggaagaaaatgaacctactcagaatcaattcggtgaaggttcactttttttctttttaaaagaatttcaagtgtgtgctgataaggttctgggaatagaaagtcaccatgattttttggtgaaagtaaaagttggaaaattcatggccaaacttgctgaacatatgttcccaaagagccaggaataacaccttgattttgtaatttagaagcaacacacattgtaaactattcattttctccaccttgtttatatggtaaaggaatcctttcagctgccagttttgaataatgaatatcatattgtatcatcaatgctgatatttaactgagttggtctttaggtttaagatggataaatgaatatcactacttgttctgaaaacatgtttgttgctttttatctcgctgcctagattgaaatattttgctatttcttctgcataagtgacagtgaaccaattcatcatgagtaagctcccttctgtcattttcattgatttaatttgtgtatcatcaataaaattgtatgttaatgctggaaagaaaaaaagaagaaagaaagaaaccatccctgtccttcagtttataatctagttggagagataagaaacgtacaaaccaaaagataacagaatatctgaagcatgtactcattgtcagatgttccctctgagagcacagaggaggcaaaagcttctgtgggatgtgctagtcggctaaagcttcacagaggaggtggcaattgaaaatgagtcctgaatggggtagggtggttagggaattccatgagacaagacaaggggggcatggtgtgagaaaggcatggaagtaggaaccctcttcctatgacaggagatcattctgcttagagtggagagtgtggagagtgggagtagataattttggaaagctgggtgaagccagttgtggagaattgtttgaatattatcccattgaatacccagagccactaaatctttttttactagaaaataattggggtccatatgaaagtctctattactgagtagtgtcaatgagggtgtggcaaaatggagcctttcacatcctagtggtggccatttggtaatacagatataagccttaaactatgtaaacccttgtcctaaggaagtaattgaataattgcccaaagattgtatgtatgaggctgttcatcccagcactgtctaagctagtaaaaattggaaacaatttaagtatctagcacattggattggttataaagcaaggaatgttcacacagtaggatattataagtatgctgatggaaatctatattgccaggaaaagctattcattatgcgttgtgaagtcagaaagtaaaaaagggtagatagaagtattcgaagtatagttccattttttgagactaataaaacatatgtttaaaaggacactaaaaactggagttatagatatccagatagaaacagtagttatctttgggtagaagaataatgagtgatctttacttttttactttttattcatctttgtgtttttatttatctaaaatgggtattgatttttaggacggttttgaaaaagaaaagtgttgggaatgaagcaagtgattgattggaaaacatactgaatggaagaaatatttagattaaaaatgaggtaggttgaagtttcttctctgaaatgatagataaatggtgaagataaggcttattgtgaggattcagtgaggtaatatatgcaaagtacttacaatgttctggcacatagtaattaattaagaaaatcgagcacccttaattacctagaatgcagggttgttagttttttggttgacttttgttttgctggggcattctgccatgttttagtgtcatttaataaataatagtaacaataaaggttaacatttattaagtgaaaaaaaaaaaaaaaaaaaaaaaaaaaaaaaaaaaaaaaaaaaaaaaaaaaaaaaa

SEQ ID NO: 4 - protein, 379 aa, Homo sapiens
MGYARKVGWVTAGLVIGAGACYCIYRLTRGRKQNKEKMAEGGSGDVDDAGDCSGARYNDWSDDDDDSNESKSIVWYPPWARIGTEAGTRTRARARARATRARLAVQKRASPNSDDTVLSPQELQKVLCLVEMSEKPYILEAALIALGNNAAYAFNRDIIRDLGGLPIVAKILNTRDPIVKEKALIVLNNLSVNAENQRRLKVYMNQVCDDTITSRLNSSVQLAGLRLLTNMTVTNEYQHMLANSISDFFRLFSAGNEETKLQVLKLLLNLAENPAMTRELLRAQVPSSLGSLFNKKENKEVILKLLVIFENINDNFKWEENEPTQNQFGEGSLFFFLKEFQVCADKVLGIESHHDFLVKVKVGKFMAKLAEHMFPKSQE

SEQ ID NO: 5 - DNA, 1608 bp, Homo sapiens
gtatcctggtgcatagacttaacactgtattttaactcaggtaatgtatggcctttttgtttatttttttcctgcatttttggggggtgttgaaataagtaaactgggaaggtgcaggggaattcttaaattcaatgcaaggagtttttgctgagtatctgcagcattcaaggaattaatattagtcactgagaacaaaaagcgaaattagaaaatttcaagtcacttctaggcttgtaggggagaagacgtgtagtgatgaattctatcatttatgaagtacccactggatcccacacactgtgcaagacctttagatcaggcgcctccctcggttttcttcaccctgtgcagcaggtgctgttatttccttttttaaattattatttattattattattttttgagacaggatctccctttgtcactcaggctggaatgcagaggcatgatcactgctcactgcagcttcgaccacccaggctcaaaggagtctcccacctcgggtgctgccacacctggccaacttttttgtatttttttggtagagaccggggtttcaccatgttgcccaggctggtcttgaacttttggactccagcgatctgcctgcctccgcctccctaagtgctgggattacagacatgagccattgtgcccgtcctgttgtttcctgtttagctgaggaggaagggttagataacttggccagtcggttgtaggaccagcactagtacagtgttgggcacgtagtaggtgtttaatacatgaccgatgagcaaatggctccagatgtctctggttccataggcagccttgaatagggctttacacacctgatgagaatgacagcctgtgttgactgagccctgacttgtgtccaaccctgccatagtgccagtgccttgcatgaattcaataatttgagcctagcagcaaccttaagaggtaggtactgttacctccccgtttataaatgagaagacaggcgcagtgaggcccaagattgaagagcttgtggccaagaagatggagttgcaggtggtttggccatagagctgatgcttgctaaatgtgttatatctgtgatggtcattttaggttaataaaagctctgtttttagattgataattctaagggtttatcatcaaggtgtatgagaaggtgagggagcccctgtgtgtagcgcagcaactctggccttctggacagtaggtaggcatgtgatcactgttgtcactaaacctgggaaatgattcctgggtcagggttcattaattgccaaatgattaaagtaataaagctgacactggaaacttatctaacttcatttcttttccttgatttacaaagatagtcaatacattttcctaccaaaaagaactggccagccgtggtggctcatgcctgtaatcctagcagtttaggaagccgaggtgggcggatcgcttgaggtcaggagttcgagaccagtctggccaacatggttgaaatcctgtctctactgaaaatacaaaaattatctgggcatagtggtgtgtgcctgtaattgcagcctgggcaacggagtgagagactgtctcaggaaaaaaaaaaaaaaaaaaaaa

SEQ ID NO: 6 - protein, 122 aa, Homo sapiens [sequence omitted]

SEQ ID NO: 7 - DNA, 1969 bp, Homo sapiens
ggaagttggtggctgcagctgccgtggttttctcctggtgtccagcagaaacggcggcggcgcaaggtgtggctgggccaacccaggatctcccaggaccctccgctctgcgcgacaaggggcccgcgcttgccaaggccgacgggcaggagtgaacgtggcctccgtgggtctgcagccccgataggccaattgtacagaatttaaaccgtctctcagatgtgtacagtagaactcaagaagacagactaccaagggtcatctgaagtcgtgattgggtcactaataacaccaggacaaagttaagggatcactactcaagcataagccccagttttcataagactgctgtgaagatgtttgatataaaggcttgggctgagtatgttgtggaatgggctgcaaaggacccctatggcttccttacaaccgttattttggcccttactccactgttcctagcaagtgctgtactgtcttggaaattggccaagatgattgaggccagggagaaggagcaaaagaagaagcaaaaacgccaagaaaacattgcaaaagctaaacgactaaaaaaggattgaaggactgaacaggctttgcaaccagaggaaaatcatttggaaaattacacagctttggaagaatccactaaagtttcttctttggatttcttgacagtatgatttagtaaatgaaatttgaccaaatggaagaatcatgttagttctgacctcaatactatagtaacttttaggcgtgggtgtagaagtttataggtttctattgacagttattgtaaattagcatttactgtggtacaaattctttataactgacttagtcatttgccgcttagcagtttatatactgaaatgaaaacatcttgtggggaaaagtgactttagattatgaactcaattcaaatgaactctatttaaaatggggtcctattttggacaaaggaaattaagaatgttaaagtcagaacagtcttgaggtaaaaagtgtgctttggcttaaaagggatacagtatattaattacatcttttattattattgtttatttcttagaatcatttctggctttctcaaaacaaaataatattaatgagtacttctatttgctgcatttttcttattacagcctttgagacagctggtaattataagtcattttccattttttaaaacataattttataaagaattctcttatctcgactatgtagaataccacctactggacagaacaatttttgtactcacaaacactgccattttcttagagatggcttgagaggagtaacactatggtttaaagcttgcagtaaaaatgccaaacactgtagtaccttggaacccagtttattcttgtgctaagcagaactgtaaaatagttaaaatgtcttatcaagtaattcgccgattacaaagacaccatttgttttttatttcattctttgttttaactcatgtggtagtgatatttaatactttctgatcaaacaggttcaaagtaaaacgttaaatttcacatttcttttaaagaactcttaaagtgtaacagttacgccatacttcataagtggtaaagaaaggtataaaatttggaaacattttgttgggcatagtagtgattgggtgaaaaggataaattatatcaaaatgagaatgtgctgtaattggaagtagggagctaaaggatgtttctttcagtttagtagaactggaacgttttactattaaacatggcttttataaatgcatggtccaataattttattcactgttagtatttaattcactgtcagcttattaatgttttctgtacccattaatgaattttaaattacmaaaaattgtctwgcagctacagtttaaaaatgaaactagacattaaaataaatttgataatttttttttaaaaaaaaaaaaaaaaaaaaaaaaaaaaaaaaaaaaaaaaaaaaa

SEQ ID NO: 8 - protein, 74 aa, Homo sapiens
MFDIKAWAEYVVEWAAKDPYGFLTTVILALTPLFLASAVLSWKLAKMIEAREKEQKKKQKRQENIAKAKRLKKD

SEQ ID NO: 9 - DNA, 819 bp, Homo sapiens
tgactttttatatatatctcagaggcaaacattcctagtgaagggttgttttcttcttgcaccttggaggggtcttttcatctgctcaggcaccttcgcatccccgtggatcagggctcagagcagaggagagtcagcagtctctaaattatcatcatctcctacctgcacatgtacacaaaaataagcctgaatgctttttcttagtatgcaatttgctgtctatttttaacttgtacacagagggccaaaaagaaaattccatgaggacatgagagtgcattgaggttgcaggtatacagtcaccaaagaacctgaaataattgccggaatgatatcctctaaaagatgtgagcctctcagagagagagagagagggttcctcttgcaacaggcatcgtgtgtgtgttttatgtcccttctcttctgctgctgtgcacttaattcggttccagccgtgtcagggagactcgagaaaaaaatcccaccattaaagacatgctctttgttttttcaatctgtgaccccagcaatctctttagcaagccatggttcagtgaactggcacacagcagccgttcggcagtggaaaaaatcataaaacagatggaagctttacatttttgtttagtttttaagagcagtttttataacatcgcttaagaccattctgatgcatcatactgtttacactcaaagctttgtagctaagatgtttacagtatggagaatgttttaagatattttatagttttgatatttagataattggcaaaaaaaaaaaaaaaaaaaaaaaaaaaaaaaaaaaaaaaaaaaaaa

SEQ ID NO: 10 - protein, 89 aa, Homo sapiens
MISSKRCEPLRERERGFLLQQASCVCFMSLLFCCCALNSVPAVSGRLEKKIPPLKTCSLFFQSVTPAISLASHGSVNWHTAAVRQWKKS

SEQ ID NO: 11 - DNA, 1969 bp, Homo sapiens
acactccatctcccgggagcaaggggaaactccgagaggagggcaacagagccagcatcttgccagggccccggaggaggggttccccgctacgcctgtgccggaggagttccagtcaccgagcgaggggcgcaagggtgggtgcatcctgcgctgcggcgggcgcgctacccagacgctggtgtgcagagccacatgaagcctgctggggactgggggccagggagcagcaagccagctgggactgaggcggacgctgtctcagggagacgctgactcgcaaagacactcccttccttgtgcctgggtaaaaagtctcctcctggggtccctggccatcctgaatatccagaatggtgtttctgaagttcttctgcatgagtttcttctgccacctgtgtcaaggctacttcgatggccccctctacccagagatgtccaatgggactctgcaccactacttcgtgcccgatggggactatgaggagaacgatgaccccgagaagtgccagctgctcttcagggtgagtgaccacaggcgctgctcccagggggaggggagccaggttggcagcctgctgagcctcaccctgcgggaggagttcaccgtgctgggccgccaggtggaggatgctgggcgcgtgctggagggcatcagcaaaagcatctcctacgacctagacggggaagagagctatggcaagtacctgcggcgggagtcccaccagatcggggatgcctactccaactcggacaaatccctcactgagctggagagcaagttcaagcagggccaggaacaggacagccggcaggagagcaggctcaacgaggactttctgggaatgctggtccacaccaggtccctgctgaaggagacactggacatctctgtggggctcagggacaaatacgagctgctggccctcaccattaggagccatgggacccgactaggtcggctgaaaaatgattatcttaaagtataggtggaaggatacaaatgctagaaagagggaatcaaatcagccccgttttggagggtgggggacagaagatggggctacatttcccccatacctactatttttttatatcccgatttgcactttgagaatacatctaaggtcatctttcaaaagagaaaaattggacacttgagtgactttgtttttagttttgtttttgtacattatttatgtgattgttatggaattgtcacctggaaagaacaattttaagcaatgtcatttctagatgggtttctaattctgcagagacacccgtttcagccacatctaaaagagcacagtttatgtggtgcggaattaaacttccccatcctgcagattatgtggaaatacccaaagataatagtgcatagctcctttcagcctctagccttcactcctgggctccaaaagctatcccagttgcctgtttttcaaatgaggttcaaggtgctgctttgcatgcctgccaacccatggaagttgtttcttacttcttttctctcttatttattaaccatggtctgagagttgtttttgttctatgtaacagtattgccacaaaactataggcaaatcgtgtttgcagggagatttctgatgcctctgtgggtgtgtgtaagttaaagtggccacatttaagaaggccaagctttgtagtggttgcacagtcacactgatatgctgatttgctctttctcattgtatgtctatgctttgtcatcagtgctatagtaaattacaaagaaataggtagattgtatgaacatacccacaaatgcctatgatttaggttaccaatgtattctttctcatttggggttttgcttctgtctgtctgtttattggaaacttgtacttcaagtagggggaatcctaattctaataactccttagctaagttttattattcaggcaataaacatgttttcatgtaaaaaaaaaaaaaaaaaaaaaaa

SEQ ID NO: 12 - protein, 211 aa, Homo sapiens
MVFLKFFCMSFFCHLCQGYFDGPLYPEMSNGTLHHYFVPDGDYEENDDPEKCQLLFRVSDHRRCSQGEGSQVGSLLSLTLREEFTVLGRQVEDAGRVLEGISKSISYDLDGEESYGKYLRRESHQIGDAYSNSDKSLTELESKFKQGQEQDSRQESRLNEDFLGMLVHTRSLLKETLDISVGLRDKYELLALTIRSHGTRLGRLKNDYLKV

SEQ ID NO: 13 - DNA, 2020 bp, Homo sapiens
ggccggaggggcagtccgccgcgggggcgagcgcgcatgcgccttcctgggacccacggcaggcgcgaatcccaacggccggcgggcggcggggatacttctacatagacataatcaagttttgactatttggaaaccaagcatcattaaaattctctcaaactcctaattgcgaagaatccataacatttcaagaagtgataacatttctctgaacaagaaaagaagtgattgaccacgttttaaaagtactctggcactggtgctgtgttttcttcccctccctaaatttgaagaactatggagaaatggtacttgatgacagtagtggttttaataggactaacagtacgatggacagtgtctcttaattcttattcaggtgctggtaaaccgcctatgtttggtgattatgaagctcagagacactggcaagaaataacttttaatttaccggtcaaacaatggtattttaacagcagtgataacaatttacagtattggggattggattacccacctcttacagcttatcatagtctcctatgtgcatatgtggcaaagtttataaatccagactggattgctctccatacatcacgtggatatgagagtcaggcacataagctcttcatgcgtacaacagttttaattgctgatctgctgatttacatacctgcagtggttttgtactgttgttgcttaaaagaaatctcaactaagaaaaagattgctaatgcattatgcatcttgctgtatccaggccttattcttatagactatggacattttcaatataattctgtgagtcttggctttgctttgtggggtgttcttggaatatcttgtgactgcgacctcctagggtcactggcattttgcttagctataaattataaacagatggaactttaccacgccttgccatttttttgctttttacttggcaagtgttttaaaaaaggcctcaaaggaaaggggtttgtgtkgctagttaagctagctkgtattgttgtggcttccttcgttctctgctggctgccattctttacagaaagggaacaaaccctgcaggttctaagaagactcttcccggttgatcgtggattatttgaggataaagtagccaatatttggtgcagcttcaatgtctttctgaagattaaggatattttgccacgtcacatccaattaataatgagcttttgttttacgtttttgagcctgcttcctgcatgcataaaattaatacttcagccctcttccaaaggattcaaatttacactggttagctgtgcgctatcattctttttattttctttccaagtacatgaaaaatccattctcttggtgtcactaccagtctgcttagttttaagtgaaattccttttatgtctacttggtttttacttgtgtcaacatttagtatgctacctcttctattgaaggatgaactcctaatgccctctgttgtgacaacaatggcattttttatagcttgtgtaacttccttttcaatatttgaaaagacttctgaagaagaactgcagttgaaatccttttccatttctgtgaggaaatatcttccatgtttwacatttctttccagaattawacaatatttgtttcttatctcagtcatcactatggtgcttctgacgttgatgactgtcacactggatcctcctcagaaactaccggacttgttttctgtattggtgtgtttkgtatcttgcttgaacttcctgttcttcttggtatactttaacattattattatgtgggattccaaaagtggaagaaatcagaagaaaatcagctagctgtattcctaaacaaattgtttcctaaacaaatgtgaaaatgtgaacagtgctgaaaggttttgtgaactttttgctatgtataaatgaaattaccattttgagaaccatggaaccacaggaaaggaaatggtgaaaagtcattgttgtctacacmaaataaatgtatatggagaccaaaaaaaaaaaaaaaaaa

SEQ ID NO: 14 - protein, 507 aa, Homo sapiens
  UNSURE (230)
  UNSURE (236)
  UNSURE (432)
  UNSURE (439)
  UNSURE (476)
MEKWYLMTVVVLIGLTVRWTVSLNSYSGAGKPPMFGDYEAQRHWQEITFNLPVKQWYFNSSDNNLQYWGLDYPPLTAYHSLLCAYVAKFINPDWIALHTSRGYESQAHKLFMRTTVLIADLLIYIPAVVLYCCCLKEISTKKKIANALCILLYPGLILIDYGHFQYNSVSLGFALWGVLGISCDCDLLGSLAFCLAINYKQMELYHALPFFCFLLGKCFKKGLKGKGFVXLVKLAXIVVASFVLCWLPFFTEREQTLQVLRRLFPVDRGLFEDKVANIWCSFNVFLKIKDILPRHIQLIMSFCFTFLSLLPACIKLILQPSSKGFKFTLVSCALSFFLFSFQVHEKSILLVSLPVCLVLSEIPFMSTWFLLVSTFSMLPLLLKDELLMPSVVTTMAFFIACVTSFSIFEKTSEEELQLKSFSISVRKYLPCXTFLSRIXQYLFLISVITMVLLTLMTVTLDPPQKLPDLFSVLVCXVSCLNFLFFLVYFNIIIMWDSKSGRNQKKIS

SEQ ID NO: 15 - DNA, 940 bp, Homo sapiens
gtttggaggtgcttgccttagagcaagggaaacagctctcattcaaaggaactagaagcctctccctcagtggtagggagacagccaggagcggttttctgggaactgtgggatgtgcccttgggggcccgagaaaacagaaggaagatgctccagaccagtaactacagcctggtgctctctctgcagttcctgctgctgtcctatgacctctttgtcaattccttctcagaactgctccaaaagactcctgtcatccagcttgtgctcttcatcatccaggatattgcagtcctcttcaacatcatcatcattttcctcatgttcttcaacaccttcgtcttccaggctggcctggtcaacctcctattccataagttcaaagggaccatcatcctgacagctgtgtactttgccctcagcatctcccttcatgtctgggtcatgaacttacgctggaaaaactccaacagcttcatatggacagatggacttcaaatgctgtttgtattccagagactagtttggaccgaattctaatttttcttgactacaagtcttcaaaataatgttttcatttttttcttcttttttccatttttttccaatttggagtcactgaaaactaagctgtgctttcataaagccctgcaaactgaatctagacaacttcagaagaaaaataacagcaacctatttacatacataagccactttcatacctgcctaccgatgtatggacttcagagtaatgtggcttatagcaattttccaggattgttcttttgtttgttgttgttctcccttcctccccctattttgtctttatgggacatgacacttcacaaccttctaaaaatgagttttcctaataactcaggacctactcgtctagaaataaaccatcctagccatgagagataagataaaaaaaaaaaaa

SEQ ID NO: 16 - protein, 130 aa, Homo sapiens
MLQTSNYSLVLSLQFLLLSYDLFVNSFSELLQKTPVIQLVLFIIQDIAVLFNIIIIFLMFFNTFVFQAGLVNLLFHKFKGTIILTAVYFALSISLHVWVMNLRWKNSNSFIWTDGLQMLFVFQRLVWTEF

SEQ ID NO: 17 - DNA, 1348 bp, Homo sapiens
gctgcttgcaggaattcaacatcatggaaaagaataaaggatgggctctcctgggaggaaaagatggccatcttcagggactatttctccttgccaacgcattgctggaaagaaatcagctccttgcacagaaggtcatgtacttattagtccctcttcttaaccgagggaatgataaacataaactcacatctgcaggcttttttgtggagcttctccggagtccagtggccaagagactgcccagcatatactctgttgcccgctttaaagactggctacaagatggaaatcatctctttagaattctcggcctgaggggactgtacaatcttgttggacaccaggagatgagagaagacatcaagagcctgttgccatacattgtagacagcttgcgtgaaaccgatgagaagatcgttctgtcagccatccagatactcctgcaacttgttagaacaatggatttcactaccctggctgccatgatgaggaccctgttctccttatttggtgatgtgagatctgatgttcatcgtttctccgtgactctctttggagccgccataaagtctgtaaaaaacccagataagaagagtatagagaaccaagtcctggacagcttggtcccactacttctgtattctcaggatgaaaatgatgcagtagctgaggagagcaggcaagtcctaactatatgtgcccagttcctgaagtggaagctgccccaagaagtgtactccaaagatccctggcacatcaaacctactgaagcaggaacaatctgcagattctttgaaaaaaagtgcaaggggaaaattaacatcctagaacaaacactgatgtactccaagaacccaaaacttcccatcagaagatcagcagtcttgtttgtaggccttttatcgaagtacatggatcacaatgagctcaggaggatgggtactgactggatagaggacgatctgagagacctgctgtgtgaccctgagccctcgctgtgcatcatcgcttcccagactctgttactagtccagatggcgagggccgaaccaaaacctaagcagagagtgaactggttgcagaagctcatgggcaggtcctctgcctagaaacacaaggcaagcaacatcagagacagaatcttgctatgttgtgcggcaagctagtcttgaactcatggcctcaagtcatcctcctgtgtcagcctcccaaagtgctgggattacaagcatgcaccacggcacccagcagaattccagtcttgagaaacaggtcaaggacagcttcaaaagagattctaaataaatgttaatgttacaatgttaaaaaaaaaaaaaaaaaaaaa

SEQ ID NO: 18 - protein, 362 aa, Homo sapiens
MEKNKGWALLGGKDGHLQGLFLLANALLERNQLLAQKVMYLLVPLLNRGNDKHKLTSAGFFVELLRSPVAKRLPSIYSVARFKDWLQDGNHLFRILGLRGLYNLVGHQEMREDIKSLLPYIVDSLRETDEKIVLSAIQILLQLVRTMDFTTLAAMMRTLFSLFGDVRSDVHRFSVTLFGAAIKSVKNPDKKSIENQVLDSLVPLLLYSQDENDAVAEESRQVLTICAQFLKWKLPQEVYSKDPWHIKPTEAGTICRFFEKKCKGKINILEQTLMYSKNPKLPIRRSAVLFVGLLSKYMDHNELRRMGTDWIEDDLRDLLCDPEPSLCIIASQTLLLVQMARAEPKPKQRVNWLQKLMGRSSA

SEQ ID NO: 19 - DNA, 1656 bp, Homo sapiens
cttctcccaccctcgctcgcgtagccatggcggagccgtcggcggccactcagtcccattccatctcctcgtcgtccttcggagccgagccgtccgcgcccggcggcggcgggagcccaggagcctgccccgccctggggacgaagagctgcagctcctcctgtgcggtgcacgatctgattttctggagagatgtgaagaagactgggtttgtctttggcaccacgctgatcatgctgctttccctggcagctttcagtgtcatcagtgtggtttcttacctcatcctggctcttctctctgtcaccatcagcttcaggatctacaagtccgtcatccaagctgtacagaagtcagaagaaggccatccattcaaagcctacctggacgtagacattactctgtcctcagaagctttccataattacatgaatgctgccatggtgcacatcaacagggccctgaaactcattattcgtctctttctggtagaagatctggttgactccttgaagctggctgtcttcatgtggctgatgacctatgttggtgctgtttttaacggaatcacccttctaattcttgctgaactgctcattttcagtgtcccgattgtctatgagaagtacaagacccagattgatcactatgttggcatcgcccgagatcagaccaagtcaattgttgaaaagatccaagcaaaactccctggaatcgccaaaaaaaaggcagaataagtacatggaaaccagaaatgcaacagttactaaaacaccatttaatagttataacgtcgttacttgtactatgaaggaaaatactcagtgtcagcttgagcctgcattccaagcttttttttttaatttggtgttttctcccatcctttccctttaaccctcagtatcaagcacaaaaattgatggactgataaaagaactatcttagaactcagaagaagaaagaatcaaattcataggataagtcaataccttaatggtggtagagcctttacctgtagcttgaaaggggaaagattggaggtaagagagaaaatgaaagaacacctctgggtccttctgtccagttttcagcactagtcttactcagctatccattatagttttgcccttaagaagtcatgattaacttatgaaaaaattatttggggacaggagtgtgataccttccttggtttttttttgcagccctcaaatcctatcttcctgccccacaatgtgagcagctacccctgatactccttttctttaatgatttaactatcaacttgataaataacttataggtgatagtgataattcctgattccaagaatgccatctgataaaaaagaatagaaatggaaagtgggactgagagggagtcagcaggcatgctgcggtggcggtcactccctctgccactatccccagggaaggaaaggctccgccatttgggaaagtggtttctacgtcactggacaccggttctgagcattagtttgagaactcgttcccgaatgtgctttcctccctctcccctgcccacctcaagtttaataaataaggttgtacttttcttactataaaataaatgtctgtaactgcaaaaaaaaaaaaaaaaaaaaaaaaaaaaaaaaaaaaaaaaaaaaaaaaaaaa

SEQ ID NO: 20 - protein, 236 aa, Homo sapiens
MAEPSAATQSHSISSSSFGAEPSAPGGGGSPGACPALGTKSCSSSCAVHDLIFWRDVKKTGFVFGTTLIMLLSLAAFSVISVVSYLILALLSVTISFRIYKSVIQAVQKSEEGHPFKAYLDVDITLSSEAFHNYMNAAMVHINRALKLIIRLFLVEDLVDSLKLAVFMWLMTYVGAVFNGITLLILAELLIFSVPIVYEKYKTQIDHYVGIARDQTKSIVEKIQAKLPGIAKKKAE

SEQ ID NO: 21 - DNA, 2439 bp, Homo sapiens
cgctttttttttttttttaaattctctttcaatccattatccattttatagctacagtgtttttttccttaacttgcagttggttacaatgcgtttattaaattcctattgtcctgacttctgtagcacatcaattccatgtgatctgatgtttgctcctctttagccttctccattatatttccttccttcgtacatttttgtttgtccatcatggttttgttttttttttttttttctttggctgttccatgctctctgccatctctagacgtttgtacaaactattcccttgagttattttctctggctcttcagctccttcctcccacctcctcccctgcaccaccaatccattcttttgcttaatttctctccatccttcaggtttcagctttaagaggtcacttcttttaggagacattccctgaatcctctcacctccacccacaaaaaaggcctctccagatgcccttcttttctgctcaaacctcatctgcttcctttatcatatgcttatcgttttggattgtaattatttatttaattgcatgtctttctgctagtttttgtgttagcaacaacaaggatcatatttttcttgttaactaatgtataacactgggtgcctaccagtaagtatttgttgaatgactaaatgagtgaaatgagttaaatgagtagaaaatatccaggaaagtagctgttttttttttttttacagtacctttgctgttgattccctgccccactttttttggtagaagtgataagctaaaatctattttcatgaatcattgtatatgttgttgtaagttgggattcatattcattccagtccattatttatatttttcaggctggcttatttattgacaaaggtgtcaaaacaaccaactctagtgctgctgacccaagggaatacctctgtttggacaataatgcaaggtaaagctggctcttttaagtcacactcaagctatactttgccaaagcaaaactttctaactggagtttattgtgctctttctgtggtaatacatttaaaataaagttttagaagttcgtaaaagggtttttggaaaaccacttattctgttgtcccaggtcctgtgactggtcttttatcttattgaaatactaaagaggttgatgtttttctcctttttcattggctataaatagatgctagagagagttgcagaaagagaaagaaaaaagaggagatatgttgagaaagtttccaaatttggtgtgcagtatgattcattgaaaaataagttaaactaagatgtttagtctcatccttgaaaaaccaaaccatatagaatttattctttgtggtcaaaatgaatactgcatttaatatttcaaaaggaatgatcccaaatttgatgagcaacctggttctggttcagccctttgtagcctcagatttacatcatagccaggctctaggttactttcttttgtaatcacaaaatgcattaggtatggatcagcatttcagcagagttctctaacatgctatatatgagccaaaataaagaaaatgtcccagatgaaaataaacttttctggaaaagatggtgacttaaagtcatattcagtagaacctgtgtacatattattattttccatactttcaaattgattgaccagtttatggagttgatagagtgctagatttaataggctttgttgacagacacttaggaaaagaataccaggcctgattgttcttcaaagaatcaagtaggaagcacaccataggttggtgagcctgacttcagggtgaggcagattctcacaacactttaataagacatgaggtcagaagaaaactggggaaagtctagtttgtcttgaggattcctcaaacatcaagttgagcctgaaatatgttgatgcaatgatcagatagcatttgagaaacccagaaactgtgggcgttttaaaaaataaaacacaaagaactggatacctaaaagctgatcaacaaatgaagtgaaaaaatagaagagataaacttaagaagaaaaaagagtatatagatgacaataaattatgaactaagaggagaatattagctggcaaataaagaattctatctagaaggttactgctgcttttttagctttgaagagtttattataattggtagaactaatataatttattataattggtagaaagtaatgtgttctttgcttagcattgccaaaaaatccataaacacaattggttctcttccctgccttaaaaacttatttatgtatatatatttatgtatatttatagtaatgtgtatgtgtatatatgtgaatatatgtgtgtatatatatgaatagagtaaatatatttgagttcattgatgttttaattagacttgacaataaaacaatttactcataaaaaaaaaaaaaaaaaaaaaaaaaa

SEQ ID NO: 22 - protein, 47 aa, Homo sapiens
MPFFSAQTSSASFIICLSFWIVIIYLIACLSASFCVSNNKDHIFLVN

SEQ ID NO: 23 - DNA, 1132 bp, Homo sapiens
  unsure (1009)
attctagacctgcggccttgaaaagacagtagtggggaaaaaaagtaccattttaccatgtgcctcaagtttgtaatactggttgcttaaacacccttcccttccacggtgggattttctcttcactttcccttgggagtctcaaatgaataagttacagtttgacagcagcagcagagcataagattttatgaatgtgaaaccattatgatctttttatttacttagaaaatttaagtgtgtgataatctttttaatagttcatttttctacatctatttctgatttcatgttgcaactatgtcatgcaaaaagacagtagatattgtaagattgtcttcaacagttgaaattcaggcttgcctttttacagtagatttcatttatagtttatacagataaatgagaactaatataaaatagtaattttgttatggcattatggtatattttaaattcatcaagctcatctgtatgtgtctttttgtccttttactactgagaggattggggctgggatcatggcagcctgctctgatgtatttctctccactctattttattatttttttaaagagttctaacttaaatacgtggaccagctattggataactttaattcatatatttatcattctttctattcactttgccacatacacaccatgtgatgattttaaacccgatttctgtatagagaatgttaaaaggatggcgtttttcagaggttccaaataggtagacattgacaatatagttgcacagtatatggaatacgtatatgtatagacatatatacacacatatacatacagatatacatatatatttctatgtatacacatatacatatatcatatatgtacacatatgcatattgcatatactgtgcaatatatatatacacacacaatttcccagttcgtatttttcattatgtcatgtaccttattgatagctattattatatggcttatgcatactgatttgaaataaacaattttacttngaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 24 - protein, 98 aa, Homo sapiens [sequence omitted]

SEQ ID NO: 25 - DNA, 401 bp, Homo sapiens
gaatcatagtgattaaaatagttggggtaaagttgtagcttatatgcaatactacttggaggaattcttctactaatttgtatttaatgtggaaattgtatagtttcattgatttaatcataaataatggaaatggtctccaagaagttttatttttcatttttttgcttatacactctgattcctataatacagtgctataagctatgcacagaaaataaaatgtttgaaatccaagaataatggttcttactgctaagagggagtaatagttattactaatgattttgattgggttgcatttttgttgcaatgtttattccacttgcagttagaatatgaatatgttttatcactagtgtggctaaataaccaaacatttgtgtaaaaaaaaaaaaaaa

SEQ ID NO: 26 - protein, 38 aa, Homo sapiens
MEMVSKKFYFSFFCLYTLIPIIQCYKLCTENKMFEIQE

SEQ ID NO: 27 - DNA, 755 bp, Homo sapiens
aaccgccaccttctaacatttaaggtagtagacagtatatacagatttgaaccttgctttttcacataatagatagttgaggtcattccatagcagtacacagaaactcatctttggtcttaaaactgcataggtactttagtcctctttgacaaatgttgggttgtttcagtcttctgctatcacaaataatgctgcaaagaatacatttgttcatatgtcatttcatccttggcaattttgcctctggaaagttcctagaagtcagattcccaggtcaaaggttaaatgcgcatgtaattttgctggatattgttaaatccccctacagagcatgcaccactcagcattcccctcagcgttgtatgagagggaccatttctccatggcctcaccagcagatttggttattgtagctctgggcttttaccaatttcacaggttaaaaatagtatctaagacaggcgtggcggctcatgcctgtaatcccagcactttgagaggccgaggaaggcagatcactggaggtcaggagttcgagatcatcctagccaacatggtgaaatcctgtctctactaaaaacataaaaattagctgggcatggtggcacatgcctgtaatcccagctactcaggaggctgaggaaggagaatatcaggaacctgggaggcaggggttgcagtgagcagagatagcgccactccactccagcctgggcgacagagtgagactctgtctcaaaaaaaaaaaaaaa

SEQ ID NO: 28 - protein, 86 aa, Homo sapiens
MLGCFSLLLSQIMLQRIHLFICHFILGNFASGKFLEVRFPGQRLNAHVILLDIVKSPYRACTTQHSPQRCMRGTISPWPHQQIWLL

SEQ ID NO: 29 - DNA, 885 bp, Homo sapiens
cgcccgagtccaagattcttcccaggaacacaaacgtaggagacccacgctcctggaagcaccagcctttatctcttcaccttcaagtcccctttctcaagaatcctctgttctttgccctctaaagtcttggtacatctaggacccaggcatcttgctttccagccacaaagagacagatgaagatgcagaaaggaaatgttctccttatgtttggtctactattgcatttagaagctgcaacaaattccaatgagactagcacctctgccaacactggatccagtgtgatctccagtggagccagcacagccaccaactctgggtccagtgtgacctccagtggggtcagcacagccaccatctcagggtccagcgtgacctccaatggggtcagcatagtcaccaactctgagttccatacaacctccagtgggatcagcacagccaccaactctgagttcagcacagcgtccagtgggatcagcatagccaccaactctgagtccagcacaacctccagtggggccagcacagccaccaactctgagtccagcacaccctccagtggggccagcacagccaccaactctgactccagcacaacctccagtggggctagcacagccaccaactctgactccagcctgggcaacaagagtggaactctgtttcaaaaaagaaagaaagaaattcagctcccacttaaagttcagttgtactctgttattgacaagtaaagtcgattgaagcccagtcatctcctgtatgttgtgtgacttctcataattatctgatcaagagtcttgaagaaacatttacaatttgatgggcaataaaataatttgaaagcaagaaaaaaaaaaaaaaaaaaaaaaaaaaaaa

SEQ ID NO: 30 - protein, 186 aa, Homo sapiens
MKMQKGNVLLMFGLLLHLEAATNSNETSTSANTGSSVISSGASTATNSGSSVTSSGVSTATISGSSVTSNGVSIVTNSEFHTTSSGISTATNSEFSTASSGISIATNSESSTTSSGASTATNSESSTPSSGASTATNSDSSTTSSGASTATNSDSSLGNKSGTLFQKRKKEIQLPLKVQLYSVIDK

SEQ ID NO: 31 - DNA, 3285 bp, Homo sapiens
gcaggtggctaaccccatttagcatctccaggccctgccatggtgtctcatcttgctgttatctctagctctttccctcctcccatttcctttagtagttgaattttgcaaagcttgtagcagtagctcagttgcctgcagcatccttgtgtgtagataaattagtcgacagaaactcagcactggggacaggattgcaaagtcggggacatagatgcagacagttgttgagatttggggatagccgggcttgtgagcggtgcccatttccagatgaagcctttcagcccttctgagtccccggcccttggtgcgatgtctgtgagtttgacctgcccagcgtgtgggctggctcaatgctgaataaagtgggtttgtgtcagctcgtttgcttcgtctccgtgtgtccacctggcctcttccccctgccctggccaccctccagtgtcaaaggaaacttcctcgtgacacgtgctaaagcatggtgaggaggactttgattgggaccattgagatgggtgtgggaccctttccttggggcctggggggagatggggctccaccccgacgtagcagggcaggggttggaggagcgaggagcagtatagggtccatgggtgggaatgactgtgaggagacatcagggctgagggggctctggctaaacccacctcacagagtccttgctgcaggcaggcagggcgatcagacattggctgcaaacggtcagagaggaacccagtcaggtaccattgagggtggtcagatattatggttaaccaaattagggttcttgctaaaactggatttcataagaaagggcaaagagggccctaggagaagattccagagcctggccagagtttggccaagtagagaatctttgtcagcacgccaacaacatcccgaccctgagacctccagtttgtctttctcactgtctccgcctgctgcagtctgctgtcatccctgagcatccctgcccctgccctgcacacctgtgatgcttgcccggacaggtcctgatggcagagtctcccacaacatcagtgtctccacatcaccaggtccgacagtggcttcaccatcctcacctaacctagctgaccagcaacatcccaccctgtcaatcacaacctctttctatttaagaaaattatatatttatggggcacagtgtgatgttttgatatctatgtacattgtggagtgacagattaatgtatccatctcatgtttttttttggtggtgagaatatttgaaatctacactcagcaatttcaaatacagtcatccctctgtgcctaagggggattggttccaggaccccctcatggataccaaaatctgcagatactcaagtaccctgcagtcagccctccctctgcacatatgtgggacagtcagatacagagggccaactgcgtacagtacacggttatcagctgaagtcaccatgctgtgcaatagaccttgagtttattcttgtatagcagggactctgtaccctctgactagaatttccccaaatcctcttgtctcagcccctgctaaccaccgttctactctctaattctataaatcaacattttgattccacatataagtgagatcatgtgatatttgtcctgttcctggcttatttcacttaatataaatgtcctgtaaattcacccatgttgcaaatggcagggtttccttttttatggccaaatagtattccatgatgtgtatacaccacattttcttaagccatttatccactttatccctttatcactttgcttctagaccacgtaggttgattccgtatcttgactgttgtaaaagtgctcttaagaaacacaggagtgtgggtatctcttccatatattcatgtcgtttcctttgggaaaatacttagcagtaggattgctgggtcacggtactctttttaagtttttgaataacctccatatgcttctccataatggctataataatttacatactcaccaacatttattttctttgaaattagtcattctaagaagtgtgagataatctcattgtgatttggtttacgtttccctgatgattaatgatgttgagcatttttttatatacctgttggccattggtatgtcttcttttgagaagtgtctcttcaggttctttgctcattttttagtcgtttatttgctttcctgctattgagtttgagttccatgtatattttggatattaaccccctacttaatgtatggtttgcaaatactctatcccaatttgtgagttgtcttcactctgtttatgatttcctttgctgtgcagaagctttttagctctatgcaatcatgtatgtttatttttcttttgttgcttgtgcttttagggtcatatgcaagaagtgatacaaccctgaaacctaggccagtgtcatggagtttttcacctgtgttttcttctactggctttacagtttcaggccttacaattaagcccttgtctattttgaatggatttttgtgtagggacattccctccacaagggcttcctctggccttgctgatgctcctccgtctcccttgtgtcctctccactccaccctcttcatgtggaagaacccttggcatcctcgtgtggcctctctgtcctatccagccccccatggtgacctcacacttgcctctctgacgtgggtctctctcccaaaccctcttccaggtccaaccactgcctccatcccagacttgcccaggggcccaatccctgcagtcctcagacatctcagagctgtctctgagttgttttctctaacagtccacaataggtctgcaaaggaatcctgcaggctcttcctgtagccaaagaccttgacctcatcttacctgctccccgccagtcccccaccgtggcccactggcactgtcctcttctgcccaggagacctggggacctcatctcctcccgctgctccaacaatgcattctcaacccagcaggtagatgggtttctactttaaaatatgtaggatgaaccagtctggtgatccgatgtacaacaggaggaatgtaggtaataaaattgcactgttttgggagttcctgctaaatgactagacttcagctgctcttgccacaaaatcctaaaagtggttgactctgggaggtgatgggaatgttaattgctcccctgtagtgaccattttgctatctgtttgtactttgtaacatcacgttgcataccttaaatatacacaatgaaatttattaaaacaatgaaaataaaaaaaaaaaaaaaa

SEQ ID NO: 32 - protein, 184 aa, Homo sapiens [sequence omitted]

SEQ ID NO: 33 - DNA, 1819 bp, Homo sapiens
aaactatatttcaagacaaccaaaagttggtgaaggaaagttgttgattagagaattccaactggttaaaaggtcaaaggaggccaggcgcggtggctcaagcctgtaatcccagcactttgggaggccgaggcaggtggatcgtgaggtcaggagatcaagaccatcctggctaacacggtgaaaccccatctctactaaaaatacaaaaaattcgccgggcgtggtggcaggcgcctgtagtcccagctactcaggaggctgaggcaggagaatggcttgaacccgggaggcggagcttgcagtgagccgagatcgcgccactgcactccagcctgggtgacagaccgagactctgtctcaaacaaaaaacaaaaaacaaaacaaaacaaagatcaaatgaatgatagaatttgaaaactacgctctttaattttacaaaatcatggattttcgtggtgatagcaatggatgcgaagaccattaggtgaaaaatggataggaagcttataatgcatggagcagaatgacaggacactaatctatattaacatctctaaatgagatcagccagatgaacttgatgtgatgaaatggatacacacagtggacacctgtgaagttttcttggctcccccaaaactgagaagtacaagttagtctccaaacctaattaccagtttacaggaaacatggggaataaaagaacaaattaacaacacaaagaagcaaacaaccaaatgcacaatttgggaaattctgcagaagtaatggcctagttttttaaccaatacatgtcaaaaaaaaaaaaaaaaaagacaaaaatggaatcctacactttaaaggagactaagaaacgtatccttcaaatacagtgtatggagcattttaggatccttgtgttaaaatgcgcttgggatttgttttaatcaatcatggtgagacaggcagacatggaaattattgtcatgaaggaagaagtttatacgcagatcccacaaacgggaggcatggcatggcatgcagggtcacgtaaagaagcacctgggtgtatcaggaggcagagggtgagagcacagcatggcccagagcttttattgggggttttcatgggaaggaatggacaaggcaggggtaggcacactggtaagcttaggattgaatagtttgagtaattttgttggtctctgggatctaggggggattcgtaattgtctagttagggcaggggaatattgaattggtgtatgagagtttggtaaaggagatagttgggagtatgggctctggattggttggtttgtatatgaaaggcatgcttgcagtggagtttatcatctatgcattagcttgccctgggaggggcagcctatccaggatcaaggccccaagtggccagagcatcaggaatacagaaaataaagaaaacatagtcaatacaagatttgaaggaataaaatgtctctacatattgtacaaatgtaaacatggattggttactaaatgatacgaaataatttattgttcatatgttaggcatgactatggcattatgggtatatgtgtatgagtccttaagtgttagagattcatactgaggtatttaaggctgaaatgttatgcctgtgatttacttttaaatacttaaaaacaaaaggtgggagggatagatgaaacaagattagcaaaatgttggtaaatgtttaatctggatcattaggtacacagggttcattgtgccgttcttactatctttacatatattttaaattttccataataaagttttttaaagtagaaaatcaaaaaaaaaaaaaaa

SEQ ID NO: 34 - protein, 75 aa, Homo sapiens
MGSGLVGLYMKGMLAVEFIIYALACPGRGSLSRIKAPSGQSIRNTENKENIVNTRFEGIKCLYILYKCKHGLVTK

SEQ ID NO: 35 - DNA, 1269 bp, Homo sapiens
gcggaggcggcgcggtggctgatcagagcgcgtagggcttcgccggggccgggagctgggcgcggtcctgctcagcccagctcaccgcgcgccggccctcggcgccctggttctgcggatcaggagaaaataatgaatgtcaaaggaaaagtaattctgtcaatgctggttgtctcaactgtgatcattgtgttttgggaatttatcaacagcacagaaggctctttcttgtggatatatcactcaaaaaacccagaagttgatgacagcagtgctcagaagggctggtggtttctgagctggtttaacaatgggatccacaattatcaacaaggggaagaagacatagacaaagaaaaaggaagagaggagaccaaaggaaggaaaatgacacaacagagcttcggctatgggactggtttaatccaaacttgaaggaatccgaataactaaactggactctggttttctgactcagtccttctagaagacctggactgagagatcatgcggttaaggagtgtgtaacaggcggaccacctgttgggactgcgagattctcaaggggaaggactgggtctcatttctcccatctcagcgcttagcaggatgacctggtatagagcagggaactgggaaatgtgggtcaggggatcagacactccagttgggtcttttatataaattaaatggcaaaaggctccatacccttctccttctttcctaccctccactttatctgcaaaatgggaatgatgataacacccacttcatagaatggtcatgaagatcaaatgagagaataaaagtcaagcacttagcctctggtgcacaataagtattaaataagtatacctattcctccttttccttttttaaaaataatattaccaaatgtccagcttatacacatttacaagacttagctagtgggctatgttagagctactaaaagatctttgacaagctaaaactaagatgcaatgaatgaggtgtaacgaacaagagagttttaagttcagaaatggttacagaagtataagacagctgtgtgggtgttttttggtttttggtttctggtttacaatctcgtcattcaacaaagatgggagttttatagaactaaaagcaccatgtaagctactaaaaacaacaacaaaaaaggctcatcatttctcagtctgaattgacaaaaatgccaatgcaaataaaaatgattactttttattttaaaaaaaaaaaaaaaaaaaaaaaaaaaaaaaaaaaaaa

SEQ ID NO: 36 - protein, 100 aa, Homo sapiens
MNVKGKVILSMLVVSTVIIVFWEFINSTEGSFLWIYHSKNPEVDDSSAQKGWWFLSWFNNGIHNYQQGEEDIDKEKGREETKGRKMTQQSFGYGTGLIQT

SEQ ID NO: 37 - DNA, 232 bp, Homo sapiens
aaaaaaaagatacttctccaaagtgttctcatgtggcctcacccaggtcttgtgtattatttggtaattaatttatggatcttaaaaactgcagtattcccccattttgtgatgagagtgtggggctggcaggggttggttggagggaggagagaagacagaggagcacttaaggtgcaaagcagcctattttttcttcaataaaaattgttaagagaaaaaaaaaaaaaaa

SEQ ID NO: 38 - protein, 57 aa, Homo sapiens
MWPHPGLVYYLVINLWILKTAVFPHFVMRVWGWQGLVGGRREDRGALKVQSSLFFLQ

SEQ ID NO: 39 - DNA, 1135 bp, Homo sapiens
ctcaaggcctggggcaggggcgtttaattgatgatgacagaggacacaggtttttgccagcaaaaaggaaaaccaargcttggtggaagggaaaggtggtgtgtcccctgttccctattccatctccctgggacttcctgctcatcatagtacccagtgagcccagagatcctactagactgggtcagcaattctagagaaccttccggaatagtctgggaacatggtcaaggtggaaggggctcccctagagagggtgggggtgtagttacttcccagttggccagaaaactgggccttgcagacccccttagcattttttccctttttttccttccctgctttctacttctttggggagccccttgtgttttggagtctgactggagtctcgcatcctggggcctgctccatccatccctcctgggcgccagaccctccatccaagccctgtgtctttccatagtcagggtcaggccctgcatctattccaaggggcactcagtacacattccataaattagctgggtgtccctgcacgcccaccccatgaaactcgagcaggtctctggaagccatttgttaaaaaaaaaaaaaaaagttttaaaaataccttttaattttctggtaattccagttctttgaagcatcctctgctgggtcttggggtgtgtggatggattggctgtctgatgggattggtaacccctcgctactcaagatggggggatacaaacaccttcagggaaggggagcctggttcttctcgttttcctttttttttttttttttaaaaaaaaactatttaattttttaatttatttttggttgttttttgcacaatgaagtttcagcttctcaaccttctcccctacccagggctgtggacccagactggccttgagccacagtccctctttccctcctcaccctcttccccctgcgggctcccgggtctgtccatttgttactgtgctgtgctggggattggcgccgaggtggcgtgagattccacttgtgtagaactttgttgagtaaagatcagtttcttgtgaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 40 - protein, 54 aa, Homo sapiens [sequence omitted]

SEQ ID NO: 41 - DNA, 4292 bp, Homo sapiens
ctcaagttaaaccaacaagccgatagaaaaaggtagttatcaagagattttaaaacttcaaccctttttctcttatagttagtgaagagagtagaatatctccagttttggctgacatctctacaacctgaacaattggcttaaacttcacttgggattcccggttgcttgttttagcatggcaaaatttggcgttcacagaatccttcttctggctatttctctgacaaagtgtctggagagtacaaaactgctggcagaccttaaaaaatgtggtgacttggaatgtgaagctttaataaacagagtctcagccatgagagattatagaggacctgactgccgatacctgaacttcactaagggagaagagatatctgtttatgttaaacttgcaggagaaagggaagatttgtgggcaggaagtaaaggaaaggagtttggatattttcccagagatgcagtccagattgaagaggtgttcatatctgaggaaattcagatgtcaacgaaagaatctgactttctttgtcttcttggagtaagttacacatttgacaatgaagatagtgaattaaacggtgattatggtgaaaatatatatccttatgaagaagataaagatgaaaaatctagtatatatgaaagtgattttcagatagaacctggattttatgcaacttatgaaagtactttgtttgaagaccaagttccagcattagaggctcctgaagatatcggaagtaccagtgaatcaaaagactgggaagaagtagttgttgaaagtatggaacaggatcgtattccagaagtgcatgtcccaccatcttcagctgtgtctggagtcaaagaatggtttggattgggaggagaacaagctgaagagaaggcttttgaatcagttattgaacctgtacaagaaagctcatttcggagtagaaaaatagcagtggaagatgagaatgacctagaggaattaaataatggtgagcctcaaacagaacatcagcaagaatctgaatcagaaattgattcagtgccaaagacacagtctgaactagcatctgagtcagagcacattcccaaacctcaatccactggttggtttggtggaggatttacaagttatttaggttttggagatgaggatacagggcttgaattaatagctgaagaaagcaatccaccactacaagattttcccaatcccatatcatctgataaagaagccacagttccatgtacagaaatattaacagaaaaaaaagacacaatcactaatgatagcttgagtctcaagccaagttggtttgattttggttttgctatactaggctttgcatatgccaaggaagataaaattatgttagatgacaggaaaaatgaagaagatggtggggcagatgaacatgaacatcctctaacaagtgaattagaccctgaaaaagaacaagaaatagaaacgataaaaattatagaaacagaagatcaaatagacaagaaaccagtctcagaaaaaacagacgaatctgatactataccatatttgaaaaagttcttgtataattttgacaacccttggaacttccagaacattccaaaggaaacagaattgccatttcccaaacagatactggatcaaaataatgtaattgaaaatgaagaaactggagaattttccattgataattatcccacagataatacaaaagttatgatattcaaaagttcatacagtctgtcagatatggtctctaacatagagttacctacgagaattcacgaagaagtatattttgaaccctcatcttctaaagatagtgatgaaaattcgaaaccatcagtagacaccgaagggcctgctctggtggagatagacagatctgtggaaaataccctgctaaatagtcagatggtttcaactgataactctttgtcttctcaaaattatatttctcagaaagaagatgcttctgagtttcagattctgaaatacttattccaaattgatgtttatgatttcatgaattctgcattttcaccaattgtaattcttacagaaagggttgtggcagcactgcctgaaggtatgagaccagattctaatctttatggttttccatgggaattggtgatatgtgcagctgttgttggattttttgctgttctcttttttttgtggagaagttttagatcggttaggagtcggctttatgtgggacgagagaaaaagcttgctctaatgctttctggactaattgaagaaaaaagtaaactacttgaaaaatttagccttgttcaaaaagagtatgaaggctatgaagtagagtcatctttaaaggatgccagctttgagaaggaggcaacagaagcacaaagtttggaggcaacctgtgaaaagctgaacaggtccaattctgaacttgaggatgaaatactctgtctagaaaaagagttaaaagaagagaaatccaaacattctgaacaagatgaattgatggcggatatttcaaaaaggatacagtctctagaagatgagtcaaaatccctcaaatcacaagtagctgaagccaaaatgaccttcaagatatttcaaatgaatgaagaacgactgaagatagcaataaaagatgctttgaatgaaaattctcaacttcaggaaagccagaaacagcttttgcaagaagctgaagtatggaaagaacaagtgagtgaacttaataaacagaaagtaacatttgaagactccaaagtacatgcagaacaagttctaaatgataaagaaagtcacatcaagactctgactgaacgcttgttaaagatgaaagattgggctgctatgcttggagaagacataacggatgatgataacttggaattagaaatgaacagtgaatcggaaaatggtgcttacttagataatcctccaaaaggagctttgaagaaactgattcatgctgctaagttaaatgcttctttaaaaaccttagaaggagaaagaaaccaaatttatattcagttgtctgaagttgataaaacaaaggaagagcttacagagcatattaaaaatcttcagactcaacaagcatctttgcagtcagaaaacacacattttgaaaatgagaatcagaagcttcaacagaaacttaaagtaatgactgaattatatcaagaaaatgaaatgaaactccaccggaaattaacagtagaggaaaattatcggttagagaaagaagagaaactttctaaagtcgacgaaaagatcagccatgccactgaagagctggagacctatagaaagcgagccaaagatcttgaagaagaattggagagaactattcattcttatcaagggcagattatttcccatgagaaaaaagcacatgataattggttggcagctcggaatgctgaaagaaacctcaatgatttaaggaaagaaaatgctcacaacagacaaaaattaactgaaacagagcttaaatttgaacttttagaaaaagatccttatgcactcgatgttccaaatacagcatttggcagaggctcacgaggcccagggaatcctctggaccatcagattaccaatgaaagaggagaatcaagctgtgataggttaaccgatcctcatagggctccctctgacactgggtctctgtcacctccatgggaccaggaccgtaggatgatgtttcctccgccaggacaatcatatcctgattcagcccttcctccacaaaggcaagacagattttgttctaattctggtagactgtctggaccagcagaactcagaagttttaatatgccttctttggataaaatggatgggtcaatgccttcagaaatggaatccagtagaaatgataccaaagatgatcttggtaatttaaatgtgcctgattcatctctccctgctgaaaatgaagccactggccctggctttgttcctccacctcttgctccaatcagaggtccattgtttccagtggatgcaagaggcccattcttgagaagaggacctcctttccccccacctcctccaggagccatgtttggagcttctcgagattattttccaccaagggatttcccaggtccaccacctgctccatttgcaatgagaaatgtctatccaccgaggggttttcctccttaccttcccccaagacctggatttttccccccacccccacattctgaaggtagaagtgagttcccctcaggtttgattccaccttcaaatgagcctgctactgaacatccagaaccacagcaagaaacctgacaat

SEQ ID NO: 42 - protein, 1369 aa, Homo sapiens
MAKFGVHRILLLAISLTKCLESTKLLADLKKCGDLECEALINRVSAMRDYRGPDCRYLNFTKGEEISVYVKLAGEREDLWAGSKGKEFGYFPRDAVQIEEVFISEEIQMSTKESDFLCLLGVSYTFDNEDSELNGDYGENIYPYEEDKDEKSSIYESDFQIEPGFYATYESTLFEDQVPALEAPEDIGSTSESKDWEEVVVESMEQDRIPEVHVPPSSAVSGVKEWFGLGGEQAEEKAFESVIEPVQESSFRSRKIAVEDENDLEELNNGEPQTEHQQESESEIDSVPKTQSELASESEHIPKPQSTGWFGGGFTSYLGFGDEDTGLELIAEESNPPLQDFPNPISSDKEATVPCTEILTEKKDTITNDSLSLKPSWFDFGFAILGFAYAKEDKIMLDDRKNEEDGGADEHEHPLTSELDPEKEQEIETIKIIETEDQIDKKPVSEKTDESDTIPYLKKFLYNFDNPWNFQNIPKETELPFPKQILDQNNVIENEETGEFSIDNYPTDNTKVMIFKSSYSLSDMVSNIELPTRIHEEVYFEPSSSKDSDENSKPSVDTEGPALVEIDRSVENTLLNSQMVSTDNSLSSQNYISQKEDASEFQILKYLFQIDVYDFMNSAFSPIVILTERVVAALPEGMRPDSNLYGFPWELVICAAVVGFFAVLFFLWRSFRSVRSRLYVGREKKLALMLSGLIEEKSKLLEKFSLVQKEYEGYEVESSLKDASFEKEATEAQSLEATCEKLNRSNSELEDEILCLEKELKEEKSKHSEQDELMADISKRIQSLEDESKSLKSQVAEAKMTFKIFQMNEERLKIAIKDALNENSQLQESQKQLLQEAEVWKEQVSELNKQKVTFEDSKVHAEQVLNDKESHIKTLTERLLKMKDWAAMLGEDITDDDNLELEMNSESENGAYLDNPPKGALKKLIHAAKLNASLKTLEGERNQIYIQLSEVDKTKEELTEHIKNLQTQQASLQSENTHFENENQKLQQKLKVMTELYQENEMKLHRKLTVEENYRLEKEEKLSKVDEKISHATEELETYRKRAKDLEEELERTIHSYQGQIISHEKKAHDNWLAARNAERNLNDLRKENAHNRQKLTETELKFELLEKDPYALDVPNTAFGRGSRGPGNPLDHQITNERGESSCDRLTDPHRAPSDTGSLSPPWDQDRRMMFPPPGQSYPDSALPPQRQDRFCSNSGRLSGPAELRSFNMPSLDKMDGSMPSEMESSRNDTKDDLGNLNVPDSSLPAENEATGPGFVPPPLAPIRGPLFPVDARGPFLRRGPPFPPPPPGAMFGASRDYFPPRDFPGPPPAPFAMRNVYPPRGFPPYLPPRPGFFPPPPHSEGRSEFPSGLIPPSNEPATEHPEPQQET

SEQ ID NO: 43 - DNA, 412 bp, Homo sapiens
ttactttatgccagatccagtacatgccattgttatcctctgtttacaggtggggaagctgaggcaggaagccctttagtcacttgccgaaggccacgctgttacccatgggaccggttttgggcggccgaagagcactcatggggccggattcacgccccgggcccgttccctcctgctctctggtgctcctcacgccattggccccactgcctctcactgcccgtgagtccctgtgcccgtgtcctccttcttgaacccctcagccctcagttaaccctcagaaagctggctcggagaagtccttgtgtggtatctgggaggcagagtttgccgtgagccgagattgtgccactgcacgcactccagcctgggcgacagagcgagaccccatctcaaaaaaaaaaaaaaa

SEQ ID NO: 44 - protein, 49 aa, Homo sapiens
MGPVLGGRRALMGPDSRPGPVPSCSLVLLTPLAPLPLTARESLCPCPPS

SEQ ID NO: 45 - DNA, 1317 bp, Homo sapiens
gtctgtgagagtcaattcaggggaaagatacaagattgatttgtaaaacccttgaaatgtagatttcttgtagatgtatccttcacgttgtaaatatgttttgtagagtgaagccatgggaagccatgtgtaacagagcttagacatccaaaactaatcaatgctgaggtggctaaatacctagccttttacatgtaaacctgtctgcaaaattagcttttttaaaaaaaaaaaaaaaaaaaaattgggggggttaatttatcattcagaaatcttgcattttcaaaaattcagtgcaagcgccaggcgatttgtgtctaaggatacgattttgaaccatatgggcagtgtacaaaatatgaaacaactgtttccacacttgcacctgatcaagagcagtgcttctccatttgttttgcagagaaatgtttttcatttcccgtgtgtttccatttccttctgaaattctgattttatccatttttttaaggctcctctttatctcctttcttaaggcactgttgctatggcacttttctataaccttttcattcctgtgtacagtagcttaaaattgcagtgattgagcataacctacttgtttgtataaattattgaaatccatttgcaccctgtaagaatggacttaaaagtactgctggacaggcatgtgtgctcaaagtacattgattgctcaaatataaggaaatggcccaatgaacgtggttgtgggaggggaaagaggaaacagagctagtcagatgtgaattgtatctgttgtaataaacatgttaaaacaaacaaaaattgttatttttcttttccttcggtcagtgcacattagcatttgaactacctggggattctttatcagaactgttcttgttgaatatttatacttaattgaaataattccttaagggaggttttgtttaaaacgtattaacaggaaattgtgtatgagatatttaatgaaataagaaattcaacaagaatgattaagtcacttcccaagtggttgtcatttgttaaaccctggtttacctgtcttgctattatgacatttcatttggaaggatgtttgtgttgtagctaactgttcaagtctggtgctgactgctgttcttagccatcacaaaacgctaaatttgtgtaattggagcttcctgctgttatctggaaatagcaggaaagcgcagctttgtatattgtttcctaaagtatattaaaataaaaaaagaaactattgctactaaaaaaaaaaaaaaaaaaaaaaaaaaaaaaaaaaaaaaaaaaaaaaaaaaaaaaaaaaaaaaa

SEQ ID NO: 46 - protein, 48 aa, Homo sapiens
MFFISRVFPFPSEILILSIFLRLLFISFLKALLLWHFSITFSFLCTVA

SEQ ID NO: 47 - DNA, 1442 bp, Homo sapiens
tgcggttgttttccttctctccgtgcaacgctggcaagtctcaaagtcgccacagaaacatgcccctgattcagtgcctctgcttagctgtaacatgttaatcagaactacctggcatcttcctgaacaagactttcaataggggccagtatgcttcgcttcatccagaagttttctcaagcatcttcaaagatactgaagtactctttcccagtgggactaagaaccagcagaacagatatactttctctcaagatgtctctccagcaaaacttttccccatgtccaaggccttggctttcctcatcatttccagcgtatatgagcaagacacagtgctatcatacatccccctgcagctttaaaaagcagcagaagcaagcacttctagccagaccctcaagcaccatcacttacctaactgacagcccaaagccagcattatgtgtaactctggcaggactaatccccttcgttgctccaccactggtcatgctgatgacaaaaacttatattcccatattagcttttactcagatggcttatggagccagtttcctatctttcttgggtgggatcagatggggttttgctctaccagaaggtagtccagccaaaccagactaccttaatttagctagcagtgcagctcctcttttcttttcatggtttgccttccttatttctgaaagacttagtgaagccatagtcacagtaataatgggtatgggagtagcattccaccttgaactttttctcttaccacattatcccaactggtttaaagccctgaggatagtagtcactttattggccactttttcatttataatcactttagtagttaaaagtagttttccagaaaaaggacataagagacctggtcaagtataaaaaatataaaagtctgggaagtgaggagcacctctgcccagctgctgccccgtctgggaagtgaggagcgcctctgcctggccgcctgaccatctgggaagtgtgacaagcgcctctgcccggccgctgtgcaaccttccacgtgtgaagtgacagccttgtgtgtgatcttttctgtcttccccaagtttgcattttcgacattaaagtttactttttagttaaaagttttaaaaatatatatatataaatacactgtagataacatttgtatgccagctacacctttttctacttctgtttggctttttttccccacaccaatggtaatttatcttcacagattgttcttcatttctagaaattgttacttcatggtaattacttgagcaaaagcttgaaaatccctgacaagtacttttcatctcatagtatattagttttcactcagtcattttatgaataatatagttatccacttaaacatttcaatattttaaccatcttgaaaattaaagattaaaaatccccttaaaaaaaaaaaaaaa

SEQ ID NO: 48 - protein, 247 aa, Homo sapiens
MLRFIQKFSQASSKILKYSFPVGLRTSRTDILSLKMSLQQNFSPCPRPWLSSSFPAYMSKTQCYHTSPCSFKKQQKQALLARPSSTITYLTDSPKPALCVTLAGLIPFVAPPLVMLMTKTYIPILAFTQMAYGASFLSFLGGIRWGFALPEGSPAKPDYLNLASSAAPLFFSWFAFLISERLSEAIVTVIMGMGVAFHLELFLLPHYPNWFKALRIVVTLLATFSFIITLVVKSSFPEKGHKRPGQV

SEQ ID NO: 49 - DNA, 2696 bp, Homo sapiens
taggcctctttggccgtggagctgtccctcctaggtgggaatgttcactcttttgttgtcaggttgtatgggggggcagggacagtgctgctgggaaggatgccagctctgggattgggccagtcctgtgggcaagacttgcaagaggctggatcaacttggtgtggtatctctgatggcttagagtaatggcaatgagggtctctgttgtgatgtcactgagtactttctggggtgcttctgggcaccccttatgatgtcacaggaagcagttcctcagaggttacttcctgtgaacataagggagcaggtacttcctgtgatgtctcaatgagttcttccttgaaggtcactttggtgatatcatgggaaggtgtacttccggtgatgcctagaggtcacgtcctgtgatgtcattaggctgaagcatgtacttcctgtattggacagtgaccagtctctgacctgccttctccctccacacccttctttggtgggtgttggcctgggggtcttcctaggaagagaataaggcacgggacttggatccaatctggaggactctaacggaaaaaaaccaatattgtcagggtcatctccattcaaacacgtcaagtctgcgaactcgccctggagggaggggtgggagatggacctagtgcaaactactgttaaagacctcccttcccacccctgccttttgtgtgcatgcctgtgtctgcgtggctttgtttcattgaatcgggtgagccaagtgtgtggtggctctgtcaggccagtatggccaggtgtagagttcagtgagccatagtagggtcccttgggccagaatgcttcgtgtggtctgataggttagattggtttggtggcttccaccaagctggcatacttcggtgatgtctgatggatcagaatgttttggtgtgctcaccaggggctctggagaactagaatgttctgatggagtctgacaagccaggcggccttgagagttggtttaggagtggctccctgagtgctgtggctgtggtcagctctaaggacctgttggcagactgagatttcagggcctgacaaccatgtagactaggatagctgagacctccctctacccccacccatctccctctcttccttggagaccacctccactttctccaacccaaagcagggcgcccagtgccctggttccatcatcagcatctctgggggaggggcgccccatgcccaccctctcccccatttgtccgcctcctaggtcttccaaacccttttcttctctatgactttgggggaaatccagcctccttgtctctctcctaaaaaaggagggaagaaaagccacagagacaattcctgcccctaaagcctaggagatccctctcccttgctagagagccacccccaaatcaaaatgtgaaaatccctagaaagcaatagccttcgaggtaccttgcactgaatttcccaccccagcccttccacccgatgggaggctgtaacttgggcactggggtgactttttccatgcccttgtcatctccagggtgggaggcaggccccacttcccctcccctatcccccacttcccattgttgttgccccacccctaatctccagactgaacccagatggagatctgagtgccaaaacaattcttgatgtaactttgtacatatcttctactaccgttgggggctcttggggttagaggtgggggcggctctgtgggccattgctcccctccacctctcaaaagaccttacagtatttcacagtatctctacccgcacgcgagtattacagtatctagctggaatatccccctacagccccccaggaccctatgaggaagggaaggagccagggagagtgaagtaaggtctgggactggggaggtgggatctgatgaactcatttgcatatcattcgcatcctccgcttggcagccgctttctacaaactcattcactggagtctgggtcccaatcagccgggtccaggactcctctcacacagacacatctccggaggctgggcctcctgaaaagtgtttgcttggggtgtctgtgtaacaacccctccctattcatatttcttggggaccccctacccagccagccagggtgatctgaaaggtatactttgctagctcagtgagctagttcactcaccatgttggtgagcagagagccacacctttccccattttaccttgggaaactcactccaccatctttgccatctcttgaaagtcccttctgcaatctgacctcaatcttttgtgctgcagtttgtccagaggggacacagatgtggggtcagggatgaggattattgaaaaacccatcatctcttttttttttccccgtctccctattagccaatccgatctcagagtctctgagtggcctccttgcaccttctcttcagcacccagtaggtgcttaataagtgtttgctgcattgaattatctccctattccttctcatttgccctctagcttcccataccttctccaagtgtcttcctccctttctttgtctggctccctatgactttctatttttttttcctccgtgtggttcccattgttttctgtcctgtctctatcttagtctttgtctgtcttcctcctttcctcaaatgtctcaactctctctccccaatttccccatttaaaaaaaaaaaaaaa

SEQ ID NO: 50 - protein, 73 aa, Homo sapiens [sequence omitted]

SEQ ID NO: 51 - DNA, 2791 bp, Homo sapiens
tttttttagagtggtaacatttattaggaaggagagacttcagttagactcccatgtctcacacagaagaaacagatggtaactgccatttctttctttctttctttctttttttttttgagatggagtttcgctcttattgcccaggctggagtgcaatggtgcaatttcggctcacctcaacctccgcctcccggtttcaagagattctcctgcctcagcctcctaagtagctgggattacaggcatacgccaccatgcctggctaattctgtatttttagtagacacggggtttctccatgttggtcaggctggtcttgaactcccgacctcaggtgatccgcccacctcggcttcccaaagtgctgggattacaagcgtgagccactgcgcccagccagtaactgccatttctaaagaggaaagagagcaggcagagggtcctgactcccaggggacaggtagttcagctggacaatgagggagtatgagattagggtggataaggacactgctcaccaccctccactgaagttcagtggctaaaatactgctacaccagccaatcagtggaaggctatcttgctctccaggggacacttgggatgtggttggtggcaggaagaaagaatgtcatgctatctcttgctgctcctccgggtttctttgccgttactaacagggttgctggaagggccaggcgggcctgcaggtgtgtggttgggctggcttcgagtgattcgggtgctgggtgggtgggagggagtatgagggggatgtgggccaccaccgggacctggtggggcactcagtccattcccattctggttctcagaggctggagaggcagcagagggtccagtggtaggctgggaagctgaagggttgctgtcgttctgcaccgccttgttgggtgcctgctgggtgctgtactctgggtttggctcactgaagttaggaacctcagaataaaccatacagaattctccaatcttctgcagatcacccccacggccagtatatagtgtcagacaacctttgaaaactgaagcgtaccctttggtgagccggataatgtccccaggctggatcagattgccaacatcgtcccagacagagatattgatgctgcctgttttgtccgccactttgcaggtccgaacctcatgcccgtcctttgtcttggtcactcggcctgtctccagcacaatgaagataaggttcagattcttgagcccaggcttgatatccttcacaaaggtctccgtcgtcatgctgcctgagcctcggcaccccactggatggggaatagaggatgaaggctcaatgcttggatcctttgcccccatgctccacttcccaaggcctacctaccccattaggatggggtcaggcagcctcaacccctctaacccttctaaaagattaaaaaagaacatccccggaggcctccaacttcaggatcagaatttgggggtctctggacaggctgctttggggctggaaggccccctgccgggatgctcttttagtctcaagccgcgaagtggcggagccgacgtagacaggggtagggaacccggtgcacagccagggagtagaatcttactggccagatcttccggaaccctcatcccgacaagccgggactcggtccatcccctcccctaccggcagcccaccacccacccaagccagccggcaggactgtgccgtggctggaagttactgtgaggcggcggctaagaaggcggttctggtggcggcggtggaggctgaggcggcggccgaggcggcgacggaggaaacagaagatggcagattttttgaaaggactgcctgtctacaacaaaagcaattttagtcgatttcacgcggactccgtgtgcaaagcctcgaaccgacggccctcagtctacctgcctacccgcgagtacccgtctgaacagatcatcgtgacagaaaagacaaacatcctcctgcgctacctgcatcagcaatgggacaaaaagaacgctgccaagaagagagaccaggagcaagtggagctcgaaggcgagagctccgcacctccccgcaaggtggcgcggaccgacagcccagacatgcacgaggacacttaagactctcaactccacaggcgcctcctgccaggtctgctcctcggtcgcccacccgcctgcccgccatgtgtaagcaccccgcccgcccgcctccctgccggcccatccacaccctgcgtccacaccacttccaacctcataggagccgatgtatttattttccttgagtttttatttatgctgtaacctgtatcaagcgttggttaaaggggacatcagacccagtagtgtgatgttggtagatgctttttaaaaaaaacaacattgtccccccgacccccgccttccatcgggccagttccccgattcctgcccccagttctccagagaaccagagtgtgtctgtgagagtctctagcgggggctttactgtggccgggcgacaggggcgggcccggggtggcctgacctaccaggacagccgagtggccttctcccccccaacaccgatccaggccattgagactcggtcttgtcccacgttcgcccggaactttcccatgcccagacctcactcagcgtgcacgcacgttggggagaagtcggcccttgggatctttctcttgagtcattttatttttatcatggactagtgcgtgctccgtgtccaccccaataaaagggtctttcctaaaaaaaaaaaaaaaaaaaaaaaaaaaaaaaaaa

SEQ ID NO: 52 - protein, 219 aa, Homo sapiens [sequence omitted]

SEQ ID NO: 53 - DNA, 1527 bp, Homo sapiens
tgaacaacaagctaaaatggaatagcacagaatggctgaggagccactgtgaagaaaggcatgcagccatgaaactgcagtgtcccttgctgttagtggggtggctctgaccaatctggaagatacagaaaatgccaagagagcctacgcagaagcagtccacctggataagtgtaaccctttagtaaacctgaactatgctgtgctgctgtacaaccagggcgagaagaagaacgccctggcccaatatcaggagatggagaagaaagtcagcctactcaaggacaatagctctctggaatttgactctgagatggtggagatggctcagaagttgggagctgctctccaggttggggaggcactggtctggaccaaaccagttaaagatcccaaatcaaagcaccagaccacttcaaccagcaaacctgccagtttccagcagcctctgggctctaatcaagctctaggacaggcaatgtcttcagcagctgcatacaggacgctcccctcaggtgctggaggaacatcccagttcacaaagcccccatctcttcctctggagccagagcctgcggtggaatcaagtccaactgaaacatcagaacaaataagagagaaataagaatagaatgaatgaccccaaaatagggttttcttgggcgaggatgtgctggattaggaaaggtgacatgacacaggcagagcagagtggcacccaccacagaatacagtgtgtgttattacgaggagccagcagttgagcctaaggtccttctacctacctggtattggcatttgaggtcggaaaccctctactgccccataagccaggaaaagtgaaaagagaacacagttcctttaagaactggcagcaaggcttgaggccttatgtatgtagctgagtcagcaaggtacatgatgctgtctgctttcaaaaggacttttctctcctagctgactgactccttccttagttcaaggaacagctgagacagacctctgctgagtagctctgtgatgacaaagccttggtttaactgaggtgatcctcaggttgtgaggtttattagtccccaaggcaaacacaaatattagattaataatccaactttaatagtatacatttaaaagaaaaaaaacaaaagccctggaagttgaggccaagcctgctgagtattgcagctgcatttgcccaaagggaatccagaacaagtccctccmtgtattttgttcttgagaggggtcagtctagaagctagatcctatcaggatgaggagcagcagcccagggcttgtctggatmagcaccaacgattttaaagaaaaaaggaagagtttcttagatgagtaattgttattgaagatagtcagtgataaccactgaccagatgctatcaatacastatgtgtcctttttagaataaagattacatatcatcatttcctttggggaaaattgttattcaggtataaaaacaagagatcataataaaaacctaaaagaacctaaaaaaaaaaaaaaaa

SEQ ID NO: 54 - protein, 122 aa, Homo sapiens
MEKKVSLLKDNSSLEFDSEMVEMAQKLGAALQVGEALVWTKPVKDPKSKHQTTSTSKPASFQQPLGSNQALGQAMSSAAAYRTLPSGAGGTSQFTKPPSLPLEPEPAVESSPTETSEQIREK

SEQ ID NO: 55 - DNA, 2352 bp, Homo sapiens
agcagagtgagctgaagctcctgaggagggttcccgaaggggggcgctcagagatgggggcagggggcggggagaggagagtctgccttatgtcccttccttgtggacttcacatggtcatgcaggaagtgaggatgggtgtccagcgggggccgaggccactagtatcctcctgcttccccctgccattctccagggctggactgaccctatggactgggagagagtgcctgaggccaccatgccacagtcaaagggggtcctatctcagaaggtggcagcatccactgagatatcctcacccgaagggaaggaggctgctgggtagcaaataagccccttcttttcttggtgagttgatgacctccaatagctcccagtgtcatgggtacccagtacgcattagctggtgttgggttgattgagacctggggcagttcctggggcaagaagccagatgggagatgagatagaaagtgttaggagttatcctctttgcctggcctttgagaataacttactgtgtgactttgggcaagttccttccccactctgggcctcagtttctcacttgggaaagcaaggagtttgaccagatgatcacaatgggccttcctagctctggccaccaagaatttgtgaacattagagctcctggtctggtgggtagagccagagctgctgactggtctctctgcctccagaggggatttattggacctcagaggtggcagggccctatggagcaccaactgccctcaaccccaccctgtgcccaagactgggaagggattgatgtcaggctgtggccataggtagcatgagttgcccaaggagggacagagcatatctttgctgaggcttggctgaggggcttatgatagggcttgcagtacctcacagccccctgtgggcacagacaccctgaggtttacccaggcaaatatattgattagcaggacaagggctctctcctcagtttctgctcccttccatctctctcccatacttgtctgaaaagggagacaaaaaatcttatacaagtggcatctcaatcctttccagtccagcacctcctggggcaggcagtgtgacttatttcctgtggtgaaatcacctgtttcacaagcctggcagcgcgacttctgagtctcatgaccactcaacccaagggacccctccccagaccagaaccaagtcagctgggggctgtcgtactaccctgtccagtcttgagggcctagttgcaggtcccccaggcatccagcccctcctagagcttgctgggcaggctgcacctcatctgggcaggcgcagagctgatgaaatgctggagcaatgcatggcaaacatatgccctccagtgtcttctgaaacctttggggctgacacaagatcctttagtgtttgggatgacctctttcctgcagacttcttcccctatccctaactcatgcatggaaaacgtttgtcaggctggtttcccgagcctcctgcacctcaacatcacgctcacccttttgggtttagcccagtgttatttagcaaatttctccagctgcagggaaggatcagagcactatcttttttttttttttttctcctggagccaggactgcacaaggcaatggccaaatttagttgaattcagcctaccatcctttgctgatgactcagctctatgccaagtactggagccacagagatgggtcagtcccagcccctgtcctcaggaagcccatggtcagggaaacgttgtagggataagtaatagagggcagttgccttcagggctcctggtggctgctggtccctatggtgccttgatgtgaattagaagacggtgccctttccaggtggattcagacctacactagaacgcacagctttgggagtgacacacaggttggattttagcaccccttgccccttggccagaggtgccctgctgcacggccatacgctgcagcctcgagggacacacaggccaaagtgtttccttcagcctcttcctggagaggaagccgcaggtcatgtttccaagcttctggtctcaaactcttggcctcaagggatcctcctacctcggcctccgaaagtgctgggattacaggtgtgagccaccatgcctggcctcactgtgtagttgtgaatagcttaatagtttgcaatgtggtgcttctcacagctcttctctgtaatgggaacatgaaaaattacctggtacagttttatgctttgtggtgtggcttttaatttttataaacatgtcttactgctattgccagggatttagatttttaataaacttccagatacaacagtaaaaaaaaaaaaaaaaaaaaaaaaaaa

SEQ ID NO: 56 - protein, 169 aa, Homo sapiens
MKCWSNAWQTYALQCLLKPLGLTQDPLVFGMTSFLQTSSPIPNSCMENVCQAGFPSLLHLNITLTLLGLAQCYLANFSSCREGSEHYLFFFFFSWSQDCTRQWPNLVEFSLPSFADDSALCQVLEPQRWVSPSPCPQEAHGQGNVVGISNRGQLPSGLLVAAGPYGALM

SEQ ID NO: 57 - DNA, 995 bp, Homo sapiens
  unsure (852)
ctaaacccttcctccagcctctacctcctgcaaaccatcgtccatattgcaagcaatcagatttatttatttattttttgaggcaggagaatggcgtgaacccgggaggcaaagcttgcagtgagccaagatcgcaccactgcactccagcctgggtgacagagcgagactctgtctcaaaaaaaaaaaaaaaaaagaaaagaaaaaaacctattgcctacctcccaagggcaaatgcagcctggtgtttggctccaagtctgcttcagctttggctcccatcactccgctttccttttgcctcaacttaagatcttgccacatgtacacttcccataacattccagctgagaggcttttgtatacgaggggtttttttttgtttgttttgccwagaatgatcctccctggtgaatcttagcttaaatcaccaggcagttaagcaggcttttctctatgatttcacccccactttgtatatttctgtgattagtcctgaacatcccatgttgtactgtttacctctctcactggacttagaaattctgaagaacagaaacaaaaagttttctctttctctgtatgttctttttttgttgttattattattgacttggtatatcttctttcagatgtattttcttttattctcaacacaaagtaattttaacatgatctttctgggccaaaattttcttatctgtaaaatgaagatgttggactaggattcagggcttcttaactaaagaattcaatagatgatgctgggacaagtgtatatctacctgtaaaggaatgaagttggaccccttcctcatactatacacaaaaattaactcaaaatggatcatngacctaaacataagagctaaaactataagactttcagaagaaaacacaggagtaagtcttcatgaccttggattaaggaatggttgcttagatatgacacccaaaaaaaaaaaaaaaaaaaaaaaaaaaaaaaaaaaaaaaaa

SEQ ID NO: 58 - protein, 72 aa, Homo sapiens
MLYCLPLSLDLEILKNRNKKFSLSLYVLFLLLLLLTWYIFFQMYFLLFSTQSNFNMIFLGQNFLICKMKMLD

SEQ ID NO: 59 - DNA, 1038 bp, Homo sapiens
gacggcctcaccatgatgaaacgggcagctgctgctgcagtgggaggaggtaagttacccggatcgcctgtctccaggccctcacctagcctggtccccgggctgctgggagaacgcagagatgaggcgctgggctggctctcaccctccacttccgaagctgcccgagtagcctgagtgagccacagcatcaaaatactccagggaaaagctcactcccattcctgacccagcttctcttctagtccttatgtcgaataagcataggaggaagatcgtttgaaagargatttgcagctaaactccacgtggcttatttcacatttatgcgtggacacacacacacacacacacacacacacacaaatttgagaccaatgaagggtattgacttcctcagcatcacacagcaagttagagacaaaccagggccatggctggtccttctatgacatctttgcttcacctggctccacactccaccttttcttcaccagaagaccactaagttgccatctctgtattgctcaagctgacagtctccggaaactgtcaaggaattcctaagcggggggcggggggaagggtcccttctcctgagcccacctctgcactcagcttctctctcccacagccctggcagtgggggctgtgcccgtggtgctcagtgccatgggcttcactggggcaggaatcgccgcgtcctccatagcagccaagatgatgtccgcagcagccattgccaacgggggtggtgtttctgcggggagcctggtggctactctgcagtccgtgggggcagctggactctccacatcatccaacatcctcctggcctctgttgggtcagtgtkgggggcctgctkggggaattcaccttcttcttctctcccagctgaacccgaggctaaagaagatgaggcaagagaaaatgtaccccaaggtgaacctccaaaacccccactcaagtcagagaaacatgaggaataaaggtcacatgcagatgcataaaaaaaaaaaaaaaaaaaaaaaaaaaaa

SEQ ID NO: 60 - protein, 105 aa, Homo sapiens
  UNSURE (61)
  UNSURE (65)
MGFTGAGIAASSIAAKMMSAAAIANGGGVSAGSLVATLQSVGAAGLSTSSNILLASVGSVXGACXGNSPSSSLPAEPEAKEDEARENVPQGEPPKPPLKSEKHEE

SEQ ID NO: 61 - DNA, 1060 bp, Homo sapiens
gaggagaccaggacagctgctgagacctctaagaagtccagatactaagagcaaagatgtttcaaactgggggcctcattgtcttctacgggctgttagcccagaccatggcccagtttggaggcctgcccgtgcccctggaccagaccctgcccttgaatgtgaatccagccctgcccttgagtcccacaggtcttgcaggaagcttgacaaatgccctcagcaatggcctgctgtctgggggcctgttgggcattctggaaaaccttccgctcctggacatcctgaagcctggaggaggtacttctggtggcctccttgggggactgcttggaaaagtgacgtcagtgattcctggcctgaacaacatcattgacataaaggtcactgacccccagctgctggaacttggccttgtgcagagccctgatggccaccgtctctatgtcaccatccctctcggcataaagctccaagtgaatacgcccctggtcggtgcaagtctgttgaggctggctgtgaagctggacatcactgcagaaatcttagctgtgagagataagcaggagaggatccacctggtccttggtgactgcacccattcccctggaagcctgcaaatttctctgcttgatggacttggccccctccccattcaaggtcttctggacagcctcacagggatcttgaataaagtcctgcctgagttggttcagggcaacgtgtgccctctggtcaatgaggttctcagaggcttggacatcaccctggtgcatgacattgttaacatgctgatccacggactacagtttgtcatcaaggtctaagccttccaggaaggggctggcctctgctgagctgaactatttcttgctgctcaatccatttcctctggcccagcttcccagtgctcacagatggctggcccatgtgctggaagatgacacagttgccttctctccgaggaacctgccccctctcctttcccaccaggcgtgtgtaacatcccatgtgcctcacctaataaaatggctcttcttctgcaaaaaaaaaaaaaaaa

SEQ ID NO: 62 - protein, 256 aa, Homo sapiens
MFQTGGLIVFYGLLAQTMAQFGGLPVPLDQTLPLNVNPALPLSPTGLAGSLTNALSNGLLSGGLLGILENLPLLDILKPGGGTSGGLLGGLLGKVTSVIPGLNNIIDIKVTDPQLLELGLVQSPDGHRLYVTIPLGIKLQVNTPLVGASLLRLAVKLDITAEILAVRDKQERIHLVLGDCTHSPGSLQISLLDGLGPLPIQGLLDSLTGILNKVLPELVQGNVCPLVNEVLRGLDITLVHDIVNMLIHGLQFVIKV

SEQ ID NO: 63 - DNA, 992 bp, Homo sapiens
gcagaatggggctctggtctctgggcattcatttccctcatagaggctgagaataaaacaaggacttattcacacatgttctagaaccccagaatggcccaagttacctgagaccagggtttctcaaccttgacaccattgacattttggactgggtaattctttgttctgcagagctgtcctttgcactgtaggagatttactaatatccctggcctctacccagtagtaccactagcacctattccccacccagcgtgtctccagatattgtcaaatatcccatcgggtgcaaaatgatccctggtcaagatctgttgcccaagatgttacaggtcacaatgaccacatttgaaattgttttccctttcattttaccctgtgaaagcatctctcctagagccttgcaagaggcaggtgacattgtgtccatatttcttcctgtttcagaacttctgtttcacaacaatttctctctcgctacaagtattctttcactcagcactggggaagttgggaacagctggtcaccatcatccctttaatcaactcacacctgtttaaagagtgtttctgatttgaccttcatcccttagtttactggggttaaaaaaagtctcagcaattttcattatttctcgtgggtctcattatcaaacctttacttatttcggcatatttcctctgggcttcttctagtttctgccttacaagcaatgctgttctgtaaatttattgaaaactctggaacatttcacctttagagatggaggatggaaggattggtaccagaagagggctaagatacgttttctgtcttgagctgaaagcacagtctactctccttcgttttgtcgatgagaaagttgaggccagaggggaggtgacatgtttagagtcacccagctggttagtgacagaaaaagcgtgagagttgtctaggattcctgccactttcaataaagacctgacttggaaaaaaaaaaaaaaaa

SEQ ID NO: 64 - protein, 82 aa, Homo sapiens [sequence omitted]

SEQ ID NO: 65 - DNA, 1095 bp, Homo sapiens
gtcttaatgagcaacagcaacagcagtctccagttaagaaagagagaattaaatacagcagagatttcctgttgaagctctcaagtgtttccatctgcagaaaaaaaccagactttctgcctgatcatcccattgtactgcaaaaaccagaaaacaaccaaagttttaagtagcattttaagaacagatgaatttaagtttggacatctgcaaatgaggtggatctagcaacaataactgtaatggactgtgacaattcaatttattcttaattttgatggttggctatttgacttctctaaaaatgagaaagagctattttaaaatataaagaattttctaatcagtttcagctttgcaggaggtttcctgcataaattgggaagtaacactggaaagtaggaatttggttagtgaagtgggaagactgtatatttataatttgcatactacttgcaattttttgtttttcatcacttgtaataatggaatggaaatgtaagctgtaaagactctcaaatataaaatatttgctacagtgtatatatggtacataattgcttgttgcttttaaagttccttctgttgttctgcttcccactgatttcataccagctcatgaatggatcattacagtctctccagaggcttagaatgattcagaatgttcaatgcatagttctcaataaacaggaggcagaatttttaatgggtatttcttttcagatatatgattggtctctaggtttttgataataatatggtcttaaattcataattactagcagagattgataatttggaaacaatggtagtgaatgaaactgaagttgaaaaacggctgctacttatgtcactaatcagaccatatgaatagcagaagttgagcaatttcaaagtaaaactgatatttttatttccaaaggaatttagacatttgaaaataattgacatacattaagttttaattcgataatttcttatatatggatgaacaatttttgggtttaagcttttaattcctagaaattttatacattaaatctcctgcaatttgtcactctggatgttactgtttaaaaaaaaaaaaaaaaa

SEQ ID NO: 66 - protein, 68 aa, Homo sapiens
MVHNCLLLLKFLLLFCFPLISYQLMNGSLQSLQRLRMIQNVQCIVLNKQEAEFLMGISFQIYDWSLGF

SEQ ID NO: 67 - DNA, 831 bp, Homo sapiens
ggctctgtgggcccagccctacccctgaagcacagttaactggttctggggtaggaactgggggccggagggacagggttctggttctggctcaaccttggctgctggtgagatccagggcctgggaaagaggggctgaggcctgaactgggcctaaggagagtgcagctcagttcgcacacaacagcacccagccctgtccccttgctgcctctacccagccctgggcagttccctcaacagagctctgcagccccaagtggcagctgctggctcaaagctgggactacatgaaagtctgaaaagagaatgagaaggaggtggcgcaagagcctggacgcacgtgtgggaggccgttttgtgcagcgctattgtgctccccgggcgggcatgtkctcgcgctccgtggctctgttggtgcccarcgtgcgggggtgtgctkgtggccctgtgggcctgtagggcaacccatgccaactgcggaaaagtaaccagcaccatacaccccccccaacacaaaactggtcatttattttttttgttgtcattgttattaggaagcaaaaaaatgtacagttacaagaatcattttccaaacagaggttaaatatgagctgaaaagtgtaaaaaaggaagaggaacatcactttacaaatcattaaattaaacaaataaacaaacagaacccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 68 - protein, 50 aa, Homo sapiens
  UNSURE (29)
  UNSURE (39)
  UNSURE (45)
MRRRWRKSLDARVGGRFVQRYCAPRAGMXSRSVALLVPXVRGCAXGPVGL

SEQ ID NO: 69 - DNA, 1893 bp, Homo sapiens
ggcaraccgtgtgagggggcctgtggccccagcgtgctgtggcctcggggagtgggaagtggaggcaggagccttccttacacttcgccatgagtttcctcatcgactccagcatcatgattacctcccaratactattttttggatttgggtggcttttcttcatgcgccaattgtttaaagactatgaratacgtcagtatgttgtacaggtgatcttctccgtgacgtttgcattttcttgcaccatgtttgagctcatcatctttgaaatcttaggagtattgaatagcagctcccgttattttcactggaaaatgaacctgtgtgtaattctgctgatcctggttttcatggtgcctttttacattggctattttattgtgagcaatatccgactactgcataaacaacgactgcttttttcctgtctcttatggctgacctttatgtatttcttctggaaactaggagatccctttcccattctcagcccaaaacatgggatcttatccatagaacagctcatcagccgggttggtgtgattggagtgactctcatggctcttctttctggatttggtgctgtcaactgcccatacacttacatgtcttacttcctcaggaatgtgactgacacggatattctagccctggaacggcgactgctgcaaaccatggatatgatcataagcaaaaagaaaaggatggcaatggcacggagaacaatgttccagaagggggaagtgcataacaaaccatcaggtttctggggaatgataaaaagtgttaccacttcagcatcaggaagtgaaaatcttactcttattcaacaggaagtggatgctttggaagaattaagcaggcagctttttctggaaacagctgatctatatgctaccaaggagagaatagaatactccaaaaccttcaaggggaaatatttaatttcttggttactttttctctatctactgtgtttggaaaattttcatgaataccatcaatattgtatttgatcgagttgggaaaacggatcctgtcacaagaggcattgagatcactgtgaattatctgggaatccaatttgatgtgaagttttggtcccaacacatttccttcattcttgttggaataatcatcgtcacatccatcagaggattgctgatcactcttaccaagttcttttatgccatctctagcagtaagtcctccaatgtcattgtcctgctattagcacagataatgggcatgtactttgtctcctctgtgctgctgatccgaatgagtatgcctttagaataccgcaccataatcactgaagtccttggagaactgcagttcaacttctatcaccgttggtttgatgtgatcttcctggtcagcgctctctctagcatactcttcctctatttggctcacaaacaggcaccagagaagcaaatggcaccttgaacttaagcctactacagactgttagaggccagtggtttcaaaatttagatataagaggggggaaaaatggaaccagggcctgacattttataaacaaacaaaatgctatggtagcatttttcaccttcatagcatactccttccccgtcaggtgatactatgaccatgagtagcatcagccagaacatgagagggagaactaactcaagacaatactcagcagagagcatcccgtgtggatatgaggctggtgtagaggcggagaggagccaagaaactaaaggtgaaaaatacactggaactctggggcaagacatgtctatggtagctgagccaaacacgtaggatttccgttttaaggttcacatggaaaaggttatagctttgccttgagattgactcattaaaatcagagactgtaacaaaaaaaaaaaaaaa

SEQ ID NO: 70 - protein, 309 aa, Homo sapiens
MSFLIDSSIMITSQILFFGFGWLFFMRQLFKDYEIRQYVVQVIFSVTFAFSCTMFELIIFEILGVLNSSSRYFHWKMNLCVILLILVFMVPFYIGYFIVSNIRLLHKQRLLFSCLLWLTFMYFFWKLGDPFPILSPKHGILSIEQLISRVGVIGVTLMALLSGFGAVNCPYTYMSYFLRNVTDTDILALERRLLQTMDMIISKKKRMAMARRTMFQKGEVHNKPSGFWGMIKSVTTSASGSENLTLIQQEVDALEELSRQLFLETADLYATKERIEYSKTFKGKYLISWLLFLYLLCLENFHEYHQYCI

SEQ ID NO: 71 - DNA, 1424 bp, Homo sapiens
cttggctgacggattgccttagaagacttcatgttattgaataacgtgaatactgtgatgatggccaattccaggtgcgcatgaagatcgtgaaaataacagctatttccagtgtttacatctacttaatattctcgtgctcagagctaacgaggctggcgttaggcggtgacgtgggcctgtttgaaggatgctggaagtcgcgggcctaggttgcatggtgtgtgtctgggctgcctcccaaaccgaggtatgtggcccagatctggctaatggacagtttcacccaagctctgtcctgtttccagctgacagctgctacctgcaggtgctgctcgagtctgtctctggttcaccataagccaaggttgggtcttctcccccaagggctcctccattccctgagacctccctgtctgggggtcctggcagcatgctatgggaggagtcctccagacatttccctcaccctcacccctcatacccctgactcaccaaaccctctagccctctggctttgttgttctgcaaaatccaacatttccttttcctacccccgcccaacctgcctaagttcagatgtccccactcctcacctccatcataaggtaagaacctgaatttgttttcccacttccttttgggcctcactcttctccaagttccccagtcacctccagaatgacttctgaacatgcaaccctcaggagtctctccgccctccccactttccccaaccctgcagtcagcaccccagggctctggaggctgtacaggtatgagatgcaaagggcctgtggtttaggtgtgagtgtggtatgggggtgtggaggcagccccgtctggcatggctgtgagggggcagtggaagacaggctgtctgtgctcccatgatggtctggggcccccctggtcagcccacatggccctgtgggggctcctgctgctacagggtgctgggctgggcggaggaagagctggccattcaggatgggcgcagtggctcatgcctgtaatcccagcactttgggaggcccaggcaggtggattgcttgagcccaggagttcaagaccagcctgggcaacatagtaaaaccccgtctttactgaaaacacaaaatttagccaggtgtggtggcgcacgcctgytactctggaggctgaggcatgagaatcgcttgaaccaggaggtggaggttgcagtgagccaaaaccatgccactgcactccagcctgggcaacagagtgagacgcggtctcaaaaaaagaagaaagaaagaaagaaagaaagaaagaaagaaataaagaaagagagagagagagagagagagagagagagaaagaaagaaagaaagawagaaagaaagaaagaaagaaagaaagaaagaaagaaaaaaaaaaaaaaaaaaaaaaaaaaaaaaaaa

SEQ ID NO: 72 - protein, 70 aa, Homo sapiens
MTSEHATLRSLSALPTFPNPAVSTPGLWRLYRYEMQRACGLGVSVVWGCGGSPVWHGCEGAVEDRLSVLP

SEQ ID NO: 73 - DNA, 1726 bp, Homo sapiens
agctggggagaaggaagaaaactgggccgggaacccctcccctcagtgtcccccagttctccatctccataaggagccatcaggctgtcattaaggaacagagtgtcactcagggggcactgtcacaaagcagcacccatggcacatgggccgggggtgcagaagcctggcttatttcaggctgacagctggaccctctgggtgcaggggctcaggcagtggccaagagcccaaagggctaaggcccgtgacgaccacccagcccgtcaccccaggtacaaacactgaccccaaagcaagagcagggactgtccctcagccctcagggccttcatgcagggtgcagaatctcatgtccacatggaggtcacccctcaggtcacacccactcccagagcaaccctgggcarggaggggcaccctggggttgtgttgaccacctccccttcaggtgaggcccttttctgccttctttctagccccctgcatggggcacctgctattgctggggctctggggtggaccctgtgtgatttctgtcagggagcttgtgctgtgcatggccagaggtgtttacatccagaagggcccagcacggccctgtggggtgtggggggaatatggtagatcattgtgatgtgcctcggggccctcttgccttggagccagctttgtttcagaatctgctacttgggccctcttcagggttttgaggctggagaagtgagttgggacagtcactgtcatcaccacccaccctgtcacccacctggaaaacattcttgatatactggccatgctgggccgggctcacatccactgagggtatagtgaccaagcatctaaaccagtcgttctcaaacttcggtgagtatcagaatcacctggaagggcttttacagattgctggccccaccccccagaatttctcatcaggagtgggcaagaccaatcatttgcatttctaacaagttcctaggagctgcagctgctggccctggaaccacactttgagaaccactgctttagaccaaacaccaaaggaagatgcagccaccctcctttacatgtcacaacgctcagggtccatgagtacctcaggctgtccagctgagctccacctgcagcagccgagattcccgactcgctccaccattgggggctaggagtgaagcgtgtcaccatggtcagctcatggccagccaggaaagcctctctgctgtgcgtctgtgcagttcttgttcttccctggaggactcttggatcgcctgtgatcttggccaggagaccaggtgcctgggtcccttcctggaaggggacaagttacacaccccagccccattttcccaccaacttctacatgccttgggagaacctgctacatgttggctgcccccttcccctatttcagcagtgcccagtcctgcttataaacctgaggcctgctccccataccctgccctgtgcaagtgccagccgttattccaggcagcccaatgttgttgaggccagatggattcctggaagcagctggcccatggatgtgagtcatcacagtattctagaaacagagaagaggtcttaacctaatgcgcatagagaaattgttctcattgtaaacatacccctgtccttagctgatctaggtggaagcccagcttcatgtgctagggggcatgataatgataataaaggaattgtatctaggaaaaaaaaaaaaaaaaaa

SEQ ID NO: 74 - protein, 133 aa, Homo sapiens
MVSSWPARKASLLCVCAVLVLPWRTLGSPVILARRPGAWVPSWKGTSYTPQPHFPTNFYMPWENLLHVGCPLPLFQQCPVLLINLRPAPHTLPCASASRYSRQPNVVEARWIPGSSWPMDVSHHSILETEKRS

SEQ ID NO: 75 - DNA, 927 bp, Homo sapiens
cagacggcggagcctggaggagcccacgcagtctgttcctggcacccggtgcgtgtgaagggacttgagggcagcgagatggaatcagcaagagaaaacatcgaccttcaacctggaagctccgaccccaggagccagcccatcaacctgaaccattacgccaccaagaagagcgtggcggagagcatgctggacgtggccctgttcatgtccaacgccatgcggctgaaggcggtgctggagcagggaccatcctctcactactacaccaccctggtcaccctcatcagcctctctctgctcctgcaggtggtcatcggtgtcctgctcgtggtcattgcacggctgaacctgaatgaggtagaaaagcagtggcgactcaaccagctcaacaacgcagccaccatcttggtcttcttcactgtggtcatcaatgttttcattacagccttcggggcacataaaacagggttcctggctgccagggcctcaaggaatcctctctgaatgcagcctgggacccaggttctggggcctggaacttctgcctccttcctccgtgatctgccaggctcggtgggcactttccacagcccaggagagcttctgaaaggacagtatagctgcccttgctccctacccacagcacctgagttaaaaagtgatttttakgttattggtctaagggacttccatcttggtctgaagtcctgagctcagacgcaggtactgccagccataccttcctggtagcatctgctggacctaagtaaggcatgtctgtctaaggccaagtctgcccggcttaaggatgctggttctgactctaccccactgcttccttctgctccaggcctcaattttcccttcttgtaaaatggaatctatatctataaaggtttcttcaaatccaaaaaaaaaaaaaaa

SEQ ID NO: 76 - protein, 142 aa, Homo sapiens
MESARENIDLQPGSSDPRSQPINLNHYATKKSVAESMLDVALFMSNAMRLKAVLEQGPSSHYYTTLVTLISLSLLLQVVIGVLLVVIARLNLNEVEKQWRLNQLNNAATILVFFTVVINVFITAFGAHKTGFLAARASRNPL

SEQ ID NO: 77 - DNA, 1660 bp, Homo sapiens
gcaagtcccacgcacagtcctgaaaaaaattttaatcttcttttcttagaactatcttggttggcatcatcaggccctgagagcacagtgcatgtcagcatctaagattccacttttcaaaatgaaggacctgatactgatcctatgcctcctggaaatgagttttgcagtgccgttctttcctcagcaatctggaacaccgggtatggctagtttgagccttgagacaatgagacagttgggaagtctgcagagattaaacacactttctcagtattctagatacggctttggaaaatcatttaattctttgtggatgcacggtctcctcccaccacattcctctcttccatggatgaggccaagagaacatgaaactcaacagtatgaatattctttgcctgtgcatcccccacctctcccatcacagccatccttgaagcctcaacagccaggactgaaaccttttctccagtctgctgctgcaaccaccaaccaggccacagcactgaaagaagcacttcagcctccaattcacctgggacatctgcccttgcaggaaggagaactgcctctggttcagcagcaggtggcaccatcagataagccaccaaagcctgagctcccaggagtagattttgctgatccacaaggtccatcactcccaggaatggattttcctgatccacaaggtccatcactcccaggattggattttgctgatccacaaggttcaacaattttccagatagcccgtttgatttctcacggaccaatgccacaaaataaacaatctccactttatccaggaatgttgtacgtgccttttggagcaaatcaattgaatgcccctgccagacttggcatcatgagttcagaagaagtggcaggcgggagagaagacccaatggcctatggagccatgtttccaggatttggaggcatgaggcccggctttgagggaatgccccacaacccagctatgggcggtgacttcactctggaatttgactccccagtggctgccaccaaaggccctgagaacgaagaaggaggtgcacaaggctcccctatgccggaggccaacccagacaatctagaaaacccagctttccttacagagctagaacctgctccccacgcagggctccttgctctccctaaggatgacattcccggcctgccaaggagcccttcagggaagatgaagggactccccagygtcaccccagcagctgctgacccactgatgacccctgaattagctgatgtttataggacctacgatgctgacatgaccacatccgtggatttccaggaagaagcaaccatggataccacgatggccccaaactctctgcaaacatccatgccaggaaacaaagcccaggagcccgagatgatgcatgacgcatggcatttccaagagccctgacagctctaagatattagctactttctgtatgcacaagcttcccagctttgtccccacagtgtacctttttgctaaaacacttattacccttctgcagcaaaggcattaaaagcgctaagcatatattaataaatgcaagtggctagaaatagtgtaggtccccttcttgctttcaatatcttgttgaaataaaatgtgtcaattgtcaaaaaaaaaaaaaaaaaa

SEQ ID NO: 78 - protein, 447 aa, Homo sapiens
MSASKIPLFKMKDLILILCLLEMSFAVPFFPQQSGTPGMASLSLETMRQLGSLQRLNTLSQYSRYGFGKSFNSLWMHGLLPPHSSLPWMRPREHETQQYEYSLPVHPPPLPSQPSLKPQQPGLKPFLQSAAATTNQATALKEALQPPIHLGHLPLQEGELPLVQQQVAPSDKPPKPELPGVDFADPQGPSLPGMDFPDPQGPSLPGLDFADPQGSTIFQIARLISHGPMPQNKQSPLYPGMLYVPFGANQLNAPARLGIMSSEEVAGGREDPMAYGAMFPGFGGMRPGFEGMPHNPAMGGDFTLEFDSPVAATKGPENEEGGAQGSPMPEANPDNLENPAFLTELEPAPHAGLLALPKDDIPGLPRSPSGKMKGLPSVTPAAADPLMTPELADVYRTYDADMTTSVDFQEEATMDTTMAPNSLQTSMPGNKAQEPEMMHDAWHFQEP

SEQ ID NO: 79 - DNA, 2036 bp, Homo sapiens
gacaaataccaagaatttttgcgtatgtttatattgtattgttctaaataatgggtagcctgtgaaataagatcttgccacccatgtaataatagtagtaatactatagttaaaatggctgtaagaatagttttataaaagtgaatacacagatctattgtatttgaaacataactttgacaattattagtgtgaccaaagtattaggcggttttcatacatttttcaccttgtacaaaattatgaattcatttttcctccaggccgacaaggagttgtagaatgaaaatgccctctaagtgttattttggttgttctaacttacaaaagtgattttgaataagaaatatttggtgttctttttataaccagtttttgattggtaattgttttctgtattgtttaaaacggatcaaaaatgtwagtctattggtagagattaagtatttattgctacmtcatagttgawaaattgatgttatcgtaaagccatatgttctgtycaagtcttgtttgccttgaaatgawtattcctacaagtgaaacactagactatttgggagtgtatatggcttgtgttttgggattttttttttttttttttggcttttgtttttgtttgtttttttgtttcgtttggtagttcatctgccttttaacccattcaccaaaatttaccttgttaacaagcatcaccaatgaacatttcagagcaatctgcatatttaacagacctaaaataaatcctattaggcaagtcagttgaaaatgctcgtgctgctaatggaattagagtgcgttcattttacaggctagtattttaaaaatagaaatcaaaatctggcaccgaagcatgctaattgtttactgtaccttgtgaggttttcactcataaatttaaaccagtgtatttttttagaactggtttgtgtatatatatagtgattatggatactaattcaatgtaatttataattttctatgtcaatacaaaaatacatcacagccttctcaaacagctcaagcaatatattgtatattgccatatcgtctggtgaaagggttaaattacttcacctcttgcacttttagatgcaaatcagtttttcatttctgtaatagaaaattattcacgtatttttacatcatttgtttttcctgaccagtatttaaaaccaaaaggatattctgaaaaatggccaacaatttttttagaagtagcatcccaagcagcgtgcctaaacattacattgcatatggaaataaaagaatcaaacgtctaatgccttatttctgatttcctttttcattttaagtggtgtggagattccagcactcccaggacagtggagtcagcagtaagccctgggacaggtggcaagggtgggtcccttgacctttgcacgccttctcaggaaccccctttcccgggtgagcccctctctgaagagactgtccttgggcctcctctggaagcagcacccccagaggacagggctcctcctgcttgcctcagggctgcctgacttgaatggcgttggacctcggggattactggtagataatatgctctggtctcgcctggtggtgagttttgccagccatggccagggtttggctccactggtggcacacgtggcctccgtggtatggacctggtggcttctccatcccactgtggcctctgtggtatggacctggtggcttctccatcctacccaaggtaacagtgtcttgcttcatcccactgactgctgggagagagcctctgggacttttctttggggcatcattttgttttgtcttttgtagcagggaaaggatatgacaatggggaggacagttcttttggaggttggaggggccaagccaaggacaggagcaagtgtgccctcattttgtttctacttttaatttctgtgtgttggccatactgaattatgagactaacagatgtctacaatacaatacctgtattcaaaataacaaaaataaagcctgattctttgtttctagaaaaaaaaaaaaaaaaaaaa

SEQ ID NO: 80 - protein, 81 aa, Homo sapiens
MLWSRLVVSFASHGQGLAPLVAHVASVVWTWWLLHPTVASVVWTWWLLHPTQGNSVLLHPTDCWERASGTFLWGIILFCLL

SEQ ID NO: 81 - DNA, 3465 bp, Homo sapiens
attttttcaaatgtaaaaataatatttttataggtatgtttgaataaaaaatgcataatcctgcctttctgttacagcttttaaaaatcagctatgtattcctttctgtttttcgtatatgtacatataaaaaaagacttttcttgttaaattctataagtaaatttctctgaaatgtcaaaaatatgaggagaagacctttcagacatatgaccttcatcaaatggtcccagtggaagaagagtaataaatgaaattaatcaagaccaagaaactaggagggcagcgggaggtaggggaataagggaaaaactattttctagttttcttacttttatgaatttaacatttttctgtaataaatgattgttaccttttcatttggtgctagaagtgggtggagtatgactgacccaagctttaaaaaaagtcaaaacaaagtagctaggaattttttttttttttttgagacagggtctcgggtgcagtggtacagtcacggctcactgcagcctggacctcctgggcccaagcaattttcccacctcagccttggcctcccaagtaggtgggactacaggtgctcaccaccatgcccagccaatgtttttattgtgtagagatggggtcttgccatgttgccaggctggtcccaaactcctgggcgcaagcagtcctcccactttggcctcccaaagtgttggaattacaggcatgagccaccacacccagcctcagagtatgttctccaacatgaccttcacctttgttttctgggaaatgtccacctcacctctggtctttcctttgttttcatactctttaaaatatccttttgttcctacagactagaggtggtgaagcagtttagtgttggccattcctctccctgccttctttagtcacagacaaggtacagatcactgaagtggagtgctagcacagacagggtgtcactcaggctaaacacttacatgtcaacctctatggcagactttacgtctcagaccctcccttctgttcatttgcctgttctttctttcttggcattggtgtgcctgtgctgtgcttgatgctgaggaagaaggactgcttttgtcccccacagtcatactgtattaatctgttttcatgctgctatgaggaactgcctgagactgggtaatttataaaggaaagaggtttaattgactcacagttcctcagggctggggaggcctcaggaaactcagtcatggcagaaggtgaaacaaacacatccttcttcacgtggtggcaggagaaagaagtgctgagcaaaagggggaagcctcttataaaaccatcagatctcgtgagaactcactcactatcatgagaagagcatggaggtaaccgcccccatgatcctattacctcctactgtgtccctcccacaacatacagggattatgggaactacaattcaagatgagatttgggtggggacacagccaaaccatatcacatgcctatagaacatggtccagctgctactctcagggataggtcagggatccagcagacaaagcagcattcgctggacattctctgaaatgtacttcttcttgsttagacaaagccttctgctcagtatcttgctttggttctgcattttgctactgttgtccacttcacttctctctccatttcttttttttttttttttttttttttgagatggagtttcgctccagcctgggtgacagagtgagactctgtatataaaacagcgatatctcaaaatgacacctaaaaatttgatgaattttaaataattggagtcatagagacacagggaaatgagaagaggaaacctggagtgaaatccatcagactgttttttgaggacactcttggcactgacctaaggtagatgacttttgcatttacctggaaggatggtcttgaattcatcattcagtatttatccatatcctgtggaatgatatagcaattgtggaggattatccgaagggtctgaaacccacacattcgtcttaaattttctgaaatttatttacttgttttaaatatgatgataagagccgcccacctgcatgggcttgtgtccctgcttttaatgtggatttatgccactgatctgcattttggacatcataagaaatactgctgtgcttcccctacacccacccctaccccacttgtttattctttgaaatggtactgagaggacttccttctcttataggagcctttgggaaaaatggaattcagtagttcaaatgtctgggcttctactgagcagataatttgtttctaacttagggcactgtcaatcctgtaattgattttttttccccctttttaagttgattcacaacaatatgtgtatcctctaaacattttttaacagctttatttagggttattaacatactataaatggtatgtttaatgtgaacaatttgataagttttgacatgtttatccctgtataaatcatcactacaatcaagatactgtgtatatccatcaacccccaacattgtctgtgctctttggcaattcctcttttctacctctccctttcccctcctgcctccatccctaggaaaccacttgtctgctttttgtcatgatagagtagtttacattttctaaaattgtatataaataggatcatgtaagtatgtacttttttggttttgcttctttctttcatcataattgtttgagatttatccatgtttttgcatgcatcagtagttcatgccttcttaatgctgagtagttttactttgtacagatgtaccaccatttgttgatccattcacttattaatggacatttgggttgttttccagttttgggcttttactcatggtacagttatgaaaatttatgtacaaatctttgcatggatatatgctttcattctcttgagtacatatctgtgagtggaattgccggatggtatggtagatatatatttaaaattttaagacaattgacatgctgtgttccgcagtggttatacatttttgcagcagtgtattagatttccagttgctgtgcaccctcaccagcacttagtatcagtctttttaactttaaccgttctagtaagtgtgtagcagctcattatggctgtgatttatatttctctaatgatattaagcatcttttcatgtgctaatttttatccatatgaaaatatggtgaaactattcaaatcttttgcccatttatttattagattgttttcttactgagttttgaaaagtttttaaagttttttttatagatttaggggtacaagtgcaactgtgttacatggatatattgtgtcttgttgaactctggattttagcatacccatcagctgaatagtataccttgtaccttgagtatttcattcctcaacccctgacccccakgtaaaaaaaaaaaaaaaaaaaaaaaaaaaaaaaaaaaaaaaaaaaa

SEQ ID NO: 82 - protein, 51 aa, Homo sapiens
MMIRAAHLHGLVSLLLMWIYATDLHFGHHKKYCCASPTPTPTPLVYSLKWY

SEQ ID NO: 83 - DNA, 808 bp, Homo sapiens
gtatgggaagaagaccctttctgagggtcacaaagggaggacctaaagctgagcagggagcacacatggaaggagaaaatccctctggcaggccagcctgcaccctcagtccaaggtgtcattggaggaactggaagctgctgcattgggggtaaccatagcaacaataaacctcaaacctagcccaactcttttttttatttactttttagagacaaggtcttgctctgttgcccaggctgaagtgcagtggtgtgatcgcagctcactgcagcctcaaactcctgggctcaagcaatcctcctgcttcagcctttgtaggagattggtcagggtggtgggagaaattataggaaagacacaaaccttcttggaaggccgagaggttttgcaaaagcttcagaaagaaattatggctgaaggcagccaaattcttatctgaagcctgagagcaaagggcagataacaggggagttgtataggaacttacctagataaatttgtttattcctgtgtccagaaaccaacctttgatcattcacacacaggactgctgtctacttgggatgttgacaatgtttattgcccacaaattgtgtttgctccaagcctttgtcattaaatttgtgctaaataaatgtgagggccaccagcttaaggggactgctaactctcttcggcccctagtgctggcagtcccctagcctgctctctcactgaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 84 - protein, 45 aa, Homo sapiens
MLTMFIAHKLCLLQAFVIKFVLNKCEGHQLKGTANSLRPLVLAVP

SEQ ID NO: 85 - DNA, 1024 bp, Homo sapiens
gaagacgcattcctttcctgccaacctctttccagataagcccttgaggtctcgggctgacctacacacacacacacacacacacacacacacacccccacacacacacacgacagagaacatgccataaacatccttgaacccatgcaggaaagcccatcccatattctgaaaaaatgccaaattaggtttttctttctttttggaaatcagtcattacagtaaccgaaaccattgggttcagcgaaaatggaaagatttagctgaatgtagtcagtccaattaagttggatgcaactgagtgatttagttgcttgggtaacccagtgcttgcttgctttcttcattctctgggtggaaactaagatcaagacacatgtttggggataagttaaatgtctgagctattttgctcggtttatcctaagagaactttattatgggatgaggaggtgacccaagatgagaagtggagggggacagcgatgttttctaaacatcgtccagtgttgactggcttccttactttgcacagtgaacacaactaaccacattaattcagctttgtgaagtccctgctctctgtgggtctatgagtcagcagcaacattggcctaacctccgtcccagcctcctggctcaccacatgtgtacagtgctgtttgcagttgtactcattatccatccatctctctgccatccccaagcatcgctgggtgtaaaacgcaaactctccaccgacactgccatgcgtagtcatgtcttgatgccttcaggggctcagtagctatcaaagaggcctggagggcctgggcaggcttgacgatgcctgaccgagttcaagacccacaccctgtagcaataccaagtgctattacataatcaatggacgatttatacttttattttttatgattatttgtttctatattgctgttagaaaaagtgaaataaaaatacttcaaaagaagatatccatataaaaataaaaggagagaaaaaaaaaaaaaaaaaaaaaaaaaaa

SEQ ID NO: 86 - protein, 64 aa, Homo sapiens
MSQQQHWPNLRPSLLAHHMCTVLFAVVLIIHPSLCHPQASLGVKRKLSTDTAMRSHVLMPSGAQ

SEQ ID NO: 87 - DNA, 867 bp, Homo sapiens
ctctgttggctgaaggaggtaactcaaacctcagggtttgtttttcccgggacagatagtagtgatagtgcattatatttgaataagaaaaacaaaccagtataccttgagaaattttaaaaagcatagttgaggcatattttttcataattatatacttatctgtttattgcccatggaaaatatatgtgtagaagtatttcttctgttatttgttactatcttcttaatttgttccaaagaaaatgctgccatactgcattccctctggaaggaaacaaaacaaaacaaaactcactcaaaaccagcagtgctgctatcagataagtagatgtcaatgtatacttacaaggaaaaactaaaaaatgtaatgtgttaattcagcctttttctatgtaatatttccaagtcagactttcttacattcctggaatttactttgatataccaagaataataatgataaaatgtttgctttgattactgtggggggaaagatgaaatgttcaattatattaaaacaaacaagcttttcagagatactggtttcctgcccttgaagggtataaagaatttagatcatgcctgtaatcccagtactttgggaggccgaggcaggtggatcacctgagatcaggagttcgagaccagcctggccaacatggcaaaaccctgtctctactaaaaatacaataattagccaggcatggtggcgggcacctgtcatcccagctacttgggaggctgaggcaggagaatcgcttgaacccaggaggcagtgattgcagtgagctgagatagcaccactgcatgcaagcctgggcaatagagcgagactccgtctcaaaaaaaaaaaaaaaaaaaaaa

SEQ ID NO: 88 - protein, 51 aa, Homo sapiens [sequence omitted]

SEQ ID NO: 89 - DNA, 1797 bp, Homo sapiens
gtctcgggctagtcatggcgtccccgtctcggagactgcagactaaaccagtcattacttgtttcaagagcgttctgctaatctacacttttattttctggatcactggcgttatccttcttgcagttggcatttggggcaaggtgagcctggagaattacttttctcttttaaatgagaaggccaccaatgtccccttcgtgctcattgctactggtaccgtcattattcttttgggcacctttggttgttttgctacctgccgagcttctgcatggatgctaaaactgtatgcaatgtttctgactctcgtttttttggtcgaactggtcgctgccatcgtaggatttgttttcagacatgagattaagaacagctttaagaataattatgagaaggctttgaagcagtataactctacaggagattatagaagccatgcagtagacaagatccaaaatacgttgcattgttgtggtgtcaccgattatagagattggacagatactaattattactcagaaaaaggatttcctaagagttgctgtaaacttgaagattgtactccacagagagatgcagacaaagtaaacaatgaaggttgttttataaaggtgatgaccattatagagtcagaaatgggagtcgttgcaggaatttcctttggagttgcttgcttccaactgattggaatctttctcgcctactgcctctctcgtgccataacaaataaccagtatgagatagtgtaacccaatgtatctgtgggcctattcctctctacctttaaggacatttagggtcccccctgtgaattagaaagttgcttggctggagaactgacaacactacttactgatagaccaaaaaactacaccagtaggttgattcaatcaagatgtatgtagacctaaaactacaccaataggctgattcaatcaagatccgtgctcgcagtgggctgattcaatcaagatgtatgtttgctatgttctaagtccaccttctatcccattcatgttagatcgttgaaaccctgtatccctctgaaacactggaagagctagtaaattgtaaatgaagtaatactgtgttcctcttgactgttatttttcttagtagggggcctttggaaggcactgtgaatttgctattttgatgtagtgttacaagatggaaaattgattcctctgactttgctattgatgtagtgtgatagaaaattcacccctctgaactggctccttcccagtcaaggttatctggtttgattgtataatttgcaccaagaagttaaaatgttttatgactctctgttctgctgacaggcagagagtcacattgtgtaatttaatttcagtcagtcaatagatggcatccctcatcagggttgccagatggtgataacagtgtaaggccttgggtctaaggcatccacgactggaagggactactgatgttctgtgatacatcaggtttcagcacacaacttacatttctttgcctccaaattgaggcatttattatgatgttcatactttccctcttgtttgaaagtttctaattattaaatggtgtcggaattgttgtattttccttaggaattcagtggaacttatcttcattaaatttagctggtaccaggttgatatgacttgtcaatattatggtcaactttaagtcttagttttcgtttgtgcctttgattaataagtataactcttatacaataaatactgctttcctctaaaaaaaaaaaaaaaaaaaaaaaaaaaaaaaaaaaaaaaaaaaaaaaaa

SEQ ID NO: 90 - protein, 245 aa, Homo sapiens
MASPSRRLQTKPVITCFKSVLLIYTFIFWITGVILLAVGIWGKVSLENYFSLLNEKATNVPFVLIATGTVIILLGTFGCFATCRASAWMLKLYAMFLTLVFLVELVAAIVGFVFRHEIKNSFKNNYEKALKQYNSTGDYRSHAVDKIQNTLHCCGVTDYRDWTDTNYYSEKGFPKSCCKLEDCTPQRDADKVNNEGCFIKVMTIIESEMGVVAGISFGVACFQLIGIFLAYCLSRAITNNQYEIV

SEQ ID NO: 91 - DNA, 1992 bp, Homo sapiens
cagaaacaccattcactccgagctgtgaccgcgcaccaacaacagcaacaactccactgcgccgggctgaggagcaggaattaggagctcgcgaataatatgaaagggatccgcaaaggggaaagccgagcaaaggaatccaaaccctgggagcctggcaagcgaagatgcgctaaatgtggccgcctagacttcatcctgatgaagaaaatggggattaaaagtggatttacgttttggaacctcgtctttttattgacggtgtcttgtgtgaaaggatttatttatacatgtggtggaactttaaaaggacttaatggcactatagaaagccctggttttccatatggatatccaaatggtgcaaactgcacatgggtaataatagcagaagaacgaaatagaatacaaattgtttttcagtcatttgctctagaagaagaatacgactacttatcattatatgatggacatcctcatcctacaaactttaggacaaggttaacaggattccatctgccacctccagtgacaagtaccaaatctgtgttctcactacgtttgaccagtgattttgcagttagtgctcatggatttaaggtatattacgaagaattgcagagtagctcttgtggaaatcctggtgttccacccaaaggtgtattatatggcacaagattcgacgtcggggacaagatccgctacagctgtgtaactggatacatccttgatggccaccctcagctcacctgcatagccaattcagttaatacagcttcgtgggattttcctgttcctatctgtagagctgaagatgcttgtggaggaacaatgagaggatccagtggcatcatatccagccctagttttcctaatgagtaccataacaatgctgattgcacttggaccattgtagcagagcctggggacacaatttcactcatatttactgattttcaaatggaagagaaatatgattacttagaaatagaaggttctgagccacctaccatatggttatctggaatgaatataccaccaccaattatcagcaacaaaaactggctcagactgcattttgttacagacagcaatcatcgataccgtggatttagtgctccctatcaagtgaaaaaggccatagattttaaatctagaggatttaaattgtttccagggaaagacaacagcaacaagttttctatcttaaatgagggaggtattaaaacagcttccaatttatgcccagatccaggagaaccagaaaatgggaagagaatcggatcagattttagccttggatcaactgtgcagttctcttgtgatgaagattatgtcctacagggcgcaaagagcatcacctgtcaacggatagctgaagtttttgctgcttggagtgatcacaggcctgtgtgtaaagtgaaaacgtgtggctctaatcttcaaggaccaagtggtacctttacatctcccaactttccgttccagtatgacagcaatgcacaatgtgtctgggtcatcacagcagtgaatacaaataaggttatccagataaattttgaagaatttgatctggagattggctatgataccttgacaattggcgatgggggcgaagttggagatcctaggacagtgctccaagtgctgactggaagctttgtaccagacttgatagtgagcatgagtagccaaatgtggctgcaccttcaaacggacgaaagtgttggatctgttggtttcaaggttaactacaaaggtaatgattaatttctacataggaaatgttatcttaataccaccagagaatatttttaaattcacgtttaattgcatctacaaaattaaaagttttgcagaacacatgctacatttcaacaaagatcatttcctccttaatttaactacaaatgttaattacacttatctttaaataaaatgagtttttcctttaaaaaaaaaaaaaaaaaaaaaaaaaaaaaaaaaaaaaaa

SEQ ID NO: 92 - protein, 556 aa, Homo sapiens
MKGIRKGESRAKESKPWEPGKRRCAKCGRLDFILMKKMGIKSGFTFWNLVFLLTVSCVKGFIYTCGGTLKGLNGTIESPGFPYGYPNGANCTWVIIAEERNRIQIVFQSFALEEEYDYLSLYDGHPHPTNFRTRLTGFHLPPPVTSTKSVFSLRLTSDFAVSAHGFKVYYEELQSSSCGNPGVPPKGVLYGTRFDVGDKIRYSCVTGYILDGHPQLTCIANSVNTASWDFPVPICRAEDACGGTMRGSSGIISSPSFPNEYHNNADCTWTIVAEPGDTISLIFTDFQMEEKYDYLEIEGSEPPTIWLSGMNIPPPIISNKNWLRLHFVTDSNHRYRGFSAPYQVKKAIDFKSRGFKLFPGKDNSNKFSILNEGGIKTASNLCPDPGEPENGKRIGSDFSLGSTVQFSCDEDYVLQGAKSITCQRIAEVFAAWSDHRPVCKVKTCGSNLQGPSGTFTSPNFPFQYDSNAQCVWVITAVNTNKVIQINFEEFDLEIGYDTLTIGDGGEVGDPRTVLQVLTGSFVPDLIVSMSSQMWLHLQTDESVGSVGFKVNYKGND

SEQ ID NO: 93 - DNA, 2085 bp, Homo sapiens
caggcgctcgcgagggggtagctcttctagtagtgctcggcgtcagacatggcggaggcgatggatttgggcaaagaccccaacgggcccacccattcctcgactctgttcgtgagggacgacggcagctccatgtccttctacgtgcggcccagcccggccaagcgtcggctgtcgacgctcatcctgcacggcggcggcaccgtgtgccgagtgcaggagcccggggccgtgctgctggcccagcccggggaggcgctggccgaggcctcgggtgatttcatctccacgcagtacatcctggactgcgtggagcgcaacgagaggctggagctggaggcctatcggctgggccccgcctcggcggcggacaccggctcggaagcaaagcccggggccctggccgagggcgccgcggagccggagccgcagcggcacgccgggcggatcgccttcacggatgcggacgacgtagccatccttacctacgtgaaggaaaatgcccgctcgcccagctccgtcaccggtaacgccttgtggaaagcgatggagaagagctcgctcacgcagcactcgtggcagtccctgaaggaccgctacctcaagcacctgcggggccaggagcataagtacctgctgggggacgcgccggtgagcccctcctcccagaagctcaagcggaaggcggaggaggacccggaggccgcggatagcggggaaccacagaataagagaactccagatttgcctgaagaagagtatgtgaaggaagaaatccaggagaatgaagaagcagtcaaaaagatgcttgtggaagccacccgggagtttgaggaggttgtggtggatgagagccctcctgattttgaaatacatataactatgtgtgatgatgatccacccacacctgaggaagactcagaaacacagcctgatgaggaggaagaagaagaagaagaaaaagtttctcaaccagaggtgggagctgccattaagatcattcggcagttaatggagaagtttaacttggatctatcaacagttacacaggccttcctaaaaaatagtggtgagctggaggctacttccgccttcttagcgtctggtcagagagctgatggatatcccatttggtcccgacaagatgacatagatttgcaaaaagatgatgaggataccagagaggcattggtcaaaaaatttggtgctcagaatgtagctcggaggattgaatttcgaaagaaataattggcaagataatgagaaaagaaaaaagtcatggtaggtgaggtggttaaaaaaaattgtgaccaatgaactttagagagttcttgcattggaactggcacttattttctgaccatcgctgctgttgctctgtgagtcctagatttttgtagccaagcagagttgtagagggggataaaaagaaaagaaattggatgtatttacagctgtccttgaacaagtatcaatgtgtttatgaaaggaagatctaaatcagacaggagttggtctacatagtagtaatccattgttggaatggaacccttgctatagtagtgacaaagtgaaaggaaatttaggaggcataggccatttcaggcagcataagtaatctcctgtcctttggcagaagctcctttagattgggatagattccaaataaagaatctagaaataggagaagatttaattatgaggccttgaacacggattatccccaaacccttgtcatttcccccagtgagctctgatttctagactgctttgaaaatgctgtattcattttgctaacttagtatttgggtaccctgctctttggctgttctttttttggagcccttctcagtcaagtctgccggatgtctttctttacctacccctcagttttccttaaaacgcgcacacaactctagagagtgttaagaataatgttacttggttaatgtgttatttattgagtattgtttgtgctaagcattgtgttagatttaaaaaattagtggattgactccactttgttgtgttgttttcattgttgaaaataaatataactttgtattcgaaaaaaaaaaaaaaaaa

SEQ ID NO: 94 - protein, 399 aa, Homo sapiens
MAEAMDLGKDPNGPTHSSTLFVRDDGSSMSFYVRPSPAKRRLSTLILHGGGTVCRVQEPGAVLLAQPGEALAEASGDFISTQYILDCVERNERLELEAYRLGPASAADTGSEAKPGALAEGAAEPEPQRHAGRIAFTDADDVAILTYVKENARSPSSVTGNALWKAMEKSSLTQHSWQSLKDRYLKHLRGQEHKYLLGDAPVSPSSQKLKRKAEEDPEAADSGEPQNKRTPDLPEEEYVKEEIQENEEAVKKMLVEATREFEEVVVDESPPDFEIHITMCDDDPPTPEEDSETQPDEEEEEEEEKVSQPEVGAAIKIIRQLMEKFNLDLSTVTQAFLKNSGELEATSAFLASGQRADGYPIWSRQDDIDLQKDDEDTREALVKKFGAQNVARRIEFRKK

SEQ ID NO: 95 - DNA, 1427 bp, Homo sapiens
ggaagtcacaagtactctctcctacttcccttaaacttacccgcattcctgaaaaagaacaataaccctgtataccttttatttacagtagacctttacacagtcaccccactatttaaactctgtctacttatgcccctaatcaccattctcacctacttctaaatgccctgcttttgtctatactgccagttcacgcttttcctccagaccattgtagctgatacctcatggagtcaccctccagctgctacccttaactctctcttagagtggatagatgaccttctgtggcaaagtactctccaattcttccatcctgatgaagttcttttcttttatacttactctttgtcttactcccgttctcctgccaccctctatccctccctaattatctcsagaataccatcaacctcacccactccctcttcaccatctccaatccttcctatgcatttccctctcttcctcmtactatacaggtgtccctgccctgccagcccastgggcaacttcccccatctccctatacctccaaacctctttcagtgacccccaactttaccctcctgaacaacttctttacttcctagaaaaattcagcaaaaactcccctgatacctcataccaacaagctgctgctctcctccatacctacctacgaaatctatctccctacgtcacttccacacctcctgttcttggacccctcactatacaaacaactatccccattgctgcccccttatgcatctccctacaattacctgctggaattcccttgggttacctcccatcttccttatgttcctttactctttacctccaaggccctgccacccacattaaccaaaatattggagcattccagcttcgtattacagaaaagccctccctcatcactaacactcttaaaaacatcagtagcaacttttgcctaggaagacatttaccctggctctcactccatccttggctatccttcccctgttcaatggattcccctccaaggccttctgcctgcctgtttatacctagtcttataaataacagtgaatggctacttacagataccaaattctttttttcacaccattaaaacagaacctctccctctacacagttatcctaccaaaccccactacaacctctaatagccgctgcccttgctggatccctagggctctgggtgcaggactctgcttccagaacacactctcattttttttactctccacttccagttatgcctgcctcatggactctttttgtttctgttggtttttccgcatacatgtgcctccctgccaattggacaggcaccttcactttaatcttccttactcccaagatcgagtttacaaatggaaacaaacaactccccattcccctcataactccaacatgacaaaaaaaaaaaaaaa

SEQ ID NO: 96 - protein, 129 aa, Homo sapiens
  UNSURE (104)
  UNSURE (115)
MPCFCLYCQFTLFLQTIVADTSWSHPPAATLNSLLEWIDDLLWQSTLQFFHPDEVLFFYTYSLSYSRSPATLYPSLIISRIPSTSPTPSSPSPILPMHFPLFLXLYRCPCPASPXGNFPHLPIPPNLFQ

SEQ ID NO: 97 - DNA, 2482 bp, Homo sapiens
  unsure (1663)
ggcgcagctctctccccctccgcagtctcagtggcgagctccgggtgctgtggcccggccttggcggggcggcctccggctcaggctggctgagaggctcccagctgcagcgtccccgcccgcctcctcgggagctctgatctcagctgacagtgccctcggggaccaaacaagcctggcaggacaaaattagaagatcaaaatggaaaatatgctgctttggttgatatttttcacccctgggtggaccctcattgatggatctgaaatggaatgggattttatgtggcacttgagaaaggtaccccggattgtcagtgaaaggactttccatctcaccagccccgcatttgaggcagatgctaagatgatggtaaatacagtgtgtggcatcgaatgccagaaagaactcccaactcccagcctttctgaattggaggattatctttcctatgagactgtctttgagaatggcacccgaaccttaaccagggtgaaagttcaagatttggttcttgagccgactcaaaatatcaccacaaagggagtatctgttaggagaaagagacaggtgtatggcaccgacagcaggttcagcatcttggacaaaaggttcttaaccaatttccctttcagcacagctgtgaagctttccacgggctgtagtggcattctcatttcccctcagcatgttctaactgctgcccactgtgttcatgatggaaaggactatgtcaaagggagtaaaaagctaagggtagggttgttgaagatgaggaataaaagtggaggcaagaaacgtcgaggttctaagaggagcaggagagaagctagtggtggtgaccaaagagagggtaccagagagcatctgcaggagagagcgaagggtgggagaagaagaaaaaaatctggccggggtcagaagattgccgaagggaggccttcctttcagtggacccgggtcaagaatacccacattccgaagggctgggcacgaggaggcatgggggacgctaccttggactatgactatgctcttctggagctgaagcgtgctcacaaaaagaaatacatggaacttggaatcagcccaacgatcaagaaaatgcctggtggaatgatccacttctcaggatttgataacgatagggctgatcagttggtctatcggttttgcagtgtgtccgacgaatccaatgatctcctttaccaatactgcgatgctgagtcgggctccaccggttcgggggtctatctgcgtctgaaagatccagacaaaaagaattggaagcgcaaaatcattgcggtctactcagggcaccagtgggtggatgtccacggggttcagaaggactacaacgttgctgttcgcatcactcccctaaaatacgcccagatttgcctctggattcacgggaacgatgccaattgtgcttacggctaacagagacctgaaacagggcggtgtatcatctaaatcacagagaaaaccagctctgcttaccgtagtgagatcacttcataggttatgcctggacttgaactctgtcaatagcatttcaacatttttcaaaatcaggagattttcgtccatttaaaaaatgtataggtgcagatattgaaactaggtgggcacttcaatgccaagtatatactcttcttnacatggtgatgagtttcatttgtagaaaaattttgttgccttcttaaaaattagacacactttaaaccttcaaacaggtattataaataacatgtgactccttaatggacttattctcagggtcctactctaagaagaatctaataggatgctggttgtgtattaaatgtgaaattgcatagataaaggtagatggtaaagcaattagtatcagaatagagacagaaagttacaacacagtttgtactactctgagatggatccattcagctcatgccctcaatgtttatattgtgttatctgttgggtctgggacatttagtttagtttttttgaagaattacaaatcagaagaaaaagcaagcattataaacaaaactaataactgttttactgctttaagaaataacaattacaatgtgtattatttaaaaatgggagaaatagtttgttctatgaaataaacctagtttagaaatagggaagctgagacattttaagatctcaagtttttatttaactaatactcaaaatatggacttttcatgtatgcatagggaagacacttcacaaattatgaatgatcatgtgttgaaagccacattattttatgctatacattctatgtatgaggtgctacatttttaggacaaagaattctgtaatctttttcaagaaagagtctttttctccttgacaaaatccagcttttgtatgaggactatagggtgaattctctgattagtaattttagatatgtcctttcctaaaaatgaataaaatttatgaatatgacttaaaaaaaaaaaaaaaaaaaaa

SEQ ID NO: 98 - protein, 413 aa, Homo sapiens
MENMLLWLIFFTPGWTLIDGSEMEWDFMWHLRKVPRIVSERTFHLTSPAFEADAKMMVNTVCGIECQKELPTPSLSELEDYLSYETVFENGTRTLTRVKVQDLVLEPTQNITTKGVSVRRKRQVYGTDSRFSILDKRFLTNFPFSTAVKLSTGCSGILISPQHVLTAAHCVHDGKDYVKGSKKLRVGLLKMRNKSGGKKRRGSKRSRREASGGDQREGTREHLQERAKGGRRRKKSGRGQKIAEGRPSFQWTRVKNTHIPKGWARGGMGDATLDYDYALLELKRAHKKKYMELGISPTIKKMPGGMIHFSGFDNDRADQLVYRFCSVSDESNDLLYQYCDAESGSTGSGVYLRLKDPDKKNWKRKIIAVYSGHQWVDVHGVQKDYNVAVRITPLKYAQICLWIHGNDANCAYG

SEQ ID NO: 99 - DNA, 2054 bp, Homo sapiens
  unsure (650)
agcctggctgtgggcccatctttggaaaaaagatctgggaatgattgtctagcctccagcctcaacttacttgatgcttgagagactcaaagccccgtggtcagctgccctgcaaagaaagtattttgaccttggcatttggacarctcccatctctcccatkgccctkacaatgctgaatgggctcctgattaaggactcaagcccwcctatgctgctgcwccaggttwacaagactgcccwgttmgatwccttcwactaccagakctgctttatgcmaagtgtctttgaccatttccctgagatcttatttatccaccsgacctataacccaaggggtaaggtcttatatwccttcctggtggatggacctcsggtgcagctggagggtcwtcttgcccgagcagtctactttgccatccctgccaaggaggacactgaaggcctggcccagatgttccaagtattcaagaagtttaatccagcatgggagagagtctgtaccatcctggtggatcctcatttccttccactgcctatcctagctatggagttccccacagctgaggtccttctctcagccttccacatttgtaagttcctccaggccaagttctatcagctgtcccttgaacggcccgtggaaaggntgctcctgacctccctgcagagcacaatgtgctcagccacagcaggcaacctgagaaagttgtatacactcctgagcaactgcatccctccagccaagctgcccgagcttcactcacactggctgctcaacgaccgcatctggctggctcaccgctggagaagccgagctgagagcagccactacttccagagcctcgaggtcaccacccacatcctcagccagttctttggtaccaccccatctgagaaacaaggtatggcttctctgttccgttacatgcagcagaactctgcagacaaggcaaacttcaaccagggcctgtgtgcccagaacaatcatgctcccccagacatcatccccgaaagccccaaactggagcagctggtagaatcccacatccagcactccctcaatgccatctgcacagggccagcagcccaactgtgcctgggcgagcttgctgtggtccagaaatccacacacctcattggctctggctcagaaaagatgaacatacagatcctggaagatacccataaggtgcagccccakccccctgccagctgcakctgctactttaaccaggccttccacctgccctgccgccacatcctagccatgctcagtgcccgccgccaggtgctccagcccgacatgctgccggctcagtggacggcaggctgtgctaccagtctagacagcatcctgggcagcaagtggagtgagaccctggataagcacctggcagtgactcacctcaccgaggaggtgggtcagctgttgcagcactgcaccaaggaggagtttgagcggaggtatagcaccctgcgggaactggccgacagctggattgggccttatgagcaggtccaactctgattattctcgatgcccagagatgctcatgcacctgtgcacactcacatccacccatacacacacacacacacacacacacacacacacactcccttacacttgtacttccgtgggccctccttccagaacaaggacaacaaggacaaggttgaagggtcttctcatctaccatggcctgcactccagcctgggagggtgagactccatctaaaaaaaataaaataaatggcaacccctggtctaagataagagataaaacatcaggtggtgaggttgaggtttggggcttggtagcagttgccccagtcatgagatgactcacttaacccgtctcctttaagtgagctgggctgggaggcttcctacaggggaagaggcccctctggggagctgactcagccaggctccctgaacttttttccttgtcccatcctggggtcaataaaactgaatgttgcatattctaaaaaaaaaaaaaaaaaaaaaaaaaaaaaaaaaaaaaaaaaaaaaaaaaaaaaaaaaa

SEQ ID NO: 100 - protein, 485 aa, Homo sapiens
  UNSURE (25)
  UNSURE (30)
  UNSURE (50)
  UNSURE (53)
  UNSURE (57)..(58)
  UNSURE (60)
  UNSURE (62)
  UNSURE (65)
  UNSURE (69)
  UNSURE (83)
  UNSURE (94)
  UNSURE (101)
  UNSURE (107)
  UNSURE (193)
  UNSURE (377)
  UNSURE (383)
MLERLKAPWSAALQRKYFDLGIWTXPISPXALTMLNGLLIKDSSPPMLLXQVXKTAXXDXFXYQXCFMXSVFDHFPEILFIHXTYNPRGKVLYXFLVDGPXVQLEGXLARAVYFAIPAKEDTEGLAQMFQVFKKFNPAWERVCTILVDPHFLPLPILAMEFPTAEVLLSAFHICKFLQAKFYQLSLERPVERXLLTSLQSTMCSATAGNLRKLYTLLSNCIPPAKLPELHSHWLLNDRIWLAHRWRSRAESSHYFQSLEVTTHILSQFFGTTPSEKQGMASLFRYMQQNSADKANFNQGLCAQNNHAPPDIIPESPKLEQLVESHIQHSLNAICTGPAAQLCLGELAVVQKSTHLIGSGSEKMNIQILEDTHKVQPXPPASCXCYFNQAFHLPCRHILAMLSARRQVLQPDMLPAQWTAGCATSLDSILGSKWSETLDKHLAVTHLTEEVGQLLQHCTKEEFERRYSTLRELADSWIGPYEQVQL

SEQ ID NO: 101 - DNA, 700 bp, Homo sapiens
gggggtttgaaaggagctgctcttgctggctccggtgcaggggatgaatgccagtgaatgccagtgttcagcagggctcctgccaggcggcactccagggtccggcccaaggtgactgtcctgaactatgcctccccgataaccgcagtcagccggccactgaatgagatggtcttgaccccactgacagagcaggagggggaagcctacctggagaagtgtggcagcgtgcggcggcacacggtggccaatgcccactcggacatccagctgctggccatggccaccatgatgcactcsggcctgggggaggaggccarcagtgagaacaagtkcctgctcctgccacccarcttccccccgccccaccsgcagtgctccagtkagcccaacatcaccgacaaccctgacggactggaggagggggccaggggcagccaggagggctcggagctgaactgtgcttccctcagctgagtcgccacccctgggcctttccatctcctgttttgcaaccaggatgrggacccctccatctccgtggattactgaggggggctcttgctttatgcgatgctgccttatttcctttagggtactgtcctggtcaaaatgacctaaggggaaaccgttgttgtaaacctttttattttggaaaaaaaaaaaaaaaaaaaaaaaaaaaaaaaaaaaaaaaaaaaaa

SEQ ID NO: 102 - protein, 139 aa, Homo sapiens
  UNSURE (88)
  UNSURE (93)
  UNSURE (99)
  UNSURE (105)
  UNSURE (110)
MPVFSRAPARRHSRVRPKVTVLNYASPITAVSRPLNEMVLTPLTEQEGEAYLEKCGSVRRHTVANAHSDIQLLAMATMMHSGLGEEAXSENKXLLLPPXFPPPHXQCSSXPNITDNPDGLEEGARGSQEGSELNCASLS

SEQ ID NO: 103 - DNA, 658 bp, Homo sapiens
cccgtcagttctgctcacgtgaggtgcttcatgaaccctctctctgctcactacctgtaacagtggtgcaaatgaatgtttatacccattttcgaggatcccatcagggacaagtgcagggcagtggcccatcagggtggtgtctacaagggaactttggtccatctctcttcagtgactggaggagcccctggccagcatccttccacacastgctgcttgcaggcacaggactggcccccaccttcccggcctccagcgtggtggcaagcctgcctgaacctgggagttcctcagggcccacttccaaatgccactgagccacagcagggaacaagaatcaaagagcaccccacccgccacccatgcctatggccccctccaagggtgtcagtggggttcagtgggccctacaggccctcctcgaatccagccccatctgcaagtcccaaagaaacttttctaaagtttctggaatgcgggtgcaaccctcactggtttttgccccatttttatgttccattcatttcactgggattctgagagggggaagataaacttgggttcaagctaccctagctgacccaggagttccatggaaacagaattctgaaaaaaaaaaaaaataaataaataaataattaaaaaaaaaaaaaaa

SEQ ID NO: 104 - protein, 155 aa, Homo sapiens
  UNSURE (46)
MFIPIFEDPIRDKCRAVAHQGGVYKGTLVHLSSVTGGAPGQHPSTXCCLQAQDWPPPSRPPAWWQACLNLGVPQGPLPNATEPQQGTRIKEHPTRHPCLWPPPRVSVGFSGPYRPSSNPAPSASPKETFLKFLECGCNPHWFLPHFYVPFISLGF

SEQ ID NO: 105 - DNA, 836 bp, Homo sapiens
atatctttatgattttctccttttctagtttgggattgacttaagcaaattagattttaaggaccaagcaactaacagaaaatacatcatggctgtacatttggaggggaaaaaaatagtgtatcatagaataattcatctcttgtcatatactttctcccagttttgacccagcaaaacaaagagaagcctcactagacaaaatgcaccttattcttacaagggtggaaacaatacattgaaatagccaggtacttgaaatgggagaaggataatgaacagcgaggacaagacagttggccatttttccgcgtctattgctctctttcttatttctgcacctttattgcttctaatgggttcaactatgtgtgtttatatttttaggaatggaggaaataccttaggaagcagatgaattattgatcatatacagaaatgatagagacagtaggaaatatgtttgatggaagccctgtgtatatatttttggggggaggggcttgaagtcacttggtacacaggtttttgggtaaggattggagaaaatgggaataaatttttctagaagcagaactatgttctgaattggcatctttgaaagggggaataaacccttaagtgggtgggactgtaactttgtttggggagacaaagaggagactctcttgagacctttattatcaggatgaggtttaaagtcagatcccaaggaaaaaacagccctagtgaaacttccaagctctttgagagttgactttttggtttggatagaaaatggaagtaaggataatagatttgactgtgtgccatggtagtggaaaaaaaaaaaaaaa

SEQ ID NO: 106 - protein, 47 aa, Homo sapiens
MNSEDKTVGHFSASIALFLISAPLLLLMGSTMCVYIFRNGGNTLGSR

SEQ ID NO: 107 - DNA, 1581 bp, Homo sapiens
agaaaaacggatcacagccactatggatgacatgttgtctactcggtctagcaccttgaccgaggatggagctaagagttcagaggccatcaaggagagcagcaagtttccatttggcattagcccagcacagagccaccggaacatcaagatcctagaggacgaaccccacagtaaggatgagaccccactgtgtacccttctggactggcaggattctcttgccaagcgctgcgtctgtgtgtccaataccattcgaagcctgtcatttgtgccaggcaatgactttgagatgtccaaacacccagggctgctgctcatcctgggcaagctgatcctgctgcaccacaagcacccagaacggaagcaggcaccactaacttatgaaaaggaggaggaacaggaccaagggtgagctgcaacaaaatggagtggtggtgggactgcttggagatgctccgggaaaacaccttggttacactcgccaacatctcggggcagttggacctatctccataccccgagagcatttgcctgcctgtcctggacggactcctacactgggcagtttgcccttcagctgaagcccaggaccccttttccaccctgggccccaatgccgtcctttccccgcagagactggtcttggaaaccctcagcaaactcagcatccaggacaacaatgtggacctgattctggccacaccccccttcagccgcctggagaagttgtatagcactatggtgcgcttcctcagtgaccgaaagaacccggtgtgccgggagatggctgtggtactgctggccaacctggctcagggggacagcctggcagctcgtgccattgcagtgcagaagggcagtatcggcaacctcctgggcttcctagaggacagccttgccgccacacagttccagcagagccaggccagcctcctccacatgcagaacccaccctttgagccaactagtgtggacatgatgcggcgggctgcccgcgcgctgcttgccttggccaaggtggacgagaaccactcagagtttactctgtacgaatcacggctgttggacatctcggtatcaccgttgatgaactcattggtttcacaagtcatttgtgatgtactgtttttgattggccagtcatgacagccgtgggacacctccccccccccgtgtgtgtgtgcgtgtgtggagaacttagaaactgactgttgccctttatttatgcaaaaccacctcagaatccagtttaccctgtgctgtccagcttctcccttgggaaaaagtctctcctgtttctctctcctccttccacctcccctccctccatcacctcacgcctttctgttccttgtcctcaccttactcccctcaggaccctaccccaccctctttgaaaagacaaagctctgcctacatagaagactttttttattttaaccaaagttactgttgtttacagtgagtttggggaaaaaaaataaaataaaaatggctttcccagtccttgcatcaacgggatgccacatttcataactgtttttaatggtaaaaaaaaaaaaaaaaaaaaaaaaaaaa

SEQ ID NO: 108 - protein, 240 aa, Homo sapiens
MEWWWDCLEMLRENTLVTLANISGQLDLSPYPESICLPVLDGLLHWAVCPSAEAQDPFSTLGPNAVLSPQRLVLETLSKLSIQDNNVDLILATPPFSRLEKLYSTMVRFLSDRKNPVCREMAVVLLANLAQGDSLAARAIAVQKGSIGNLLGFLEDSLAATQFQQSQASLLHMQNPPFEPTSVDMMRRAARALLALAKVDENHSEFTLYESRLLDISVSPLMNSLVSQVICDVLFLIGQS

SEQ ID NO: 109 - DNA, 1684 bp, Homo sapiens
ctgcctgatttgggaagcgctgcaaggacaaccggctggggtccttgcgcgccgcggctcagggaggagcaccgactgcgccgcaccctgagagatggttggtgccatgtggaaggtgattgtttcgctggtcctgttgatgcctggcccctgtgatgggctgtttcactccctatacagaagtgtttccatgccacctaagggagactcaggacagccattatttctcaccccttacattgaagctgggaagatccaaaaaggaagagaattgagtttggtcggtcctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtacctaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgraaaaaaaaaaaaaaaaaaaaaaa

SEQ ID NO: 110 - protein, 476 aa, Homo sapiens
MVGAMWKVIVSLVLLMPGPCDGLFHSLYRSVSMPPKGDSGQPLFLTPYIEAGKIQKGRELSLVGPFPGLNMKSYAGFLTVNKTYNSNLFFWFFPAQIQPEDAPVVLWLQGGPGGSSMFGLFVEHGPYVVTSNMTLRDRDFPWTTTLSMLYIDNPVGTGFSFTDDTHGYAVNEDDVARDLYSALIQFFQIFPEYKNNDFYVTGESYAGKYVPAIAHLIHSLNPVREVKINLNGIAIGDGYSDPESIIGGYAEFLYLIGLLDEKQKKYFQKQCHECIEHIRKQNWFEAFEILDKLLDGDLTSDPSYFQNVTGCSNYYNFLRCTEPEDQLYYVKFLSLPEVRQAIHVGNQTFNDGTIVEKYLREDTVQSVKPWLTEIMNNYKVLIYNGQLDIIVAAALTERSLMGMDWKGSQEYKKAEKKVWKIFKSDSEVAGYIRQAGDFHQVIIRGGGHILPYDQPLRAFDMINRFIYGKGWDPYVG

SEQ ID NO: 111 - DNA, 750 bp, Homo sapiens
acgatgtgttgaccggctgccgtttgaggactttggtcacccagactagacaccttctgtgctcatgtttggaaagctgaaagggaaggacagctgtgccctcctgggagctcatgtgtccctggcgctgtgctagctttcctttacagctgtttacagacaaggcaggcctgaggcagatggccactgctcttgtgatgtttgctcagaggaatatgaacattttatttttgaaaagggatgatgtggtttttgccaggtgtttataattaatcctttaatattatggttattaacctcttaaacatgaatgaattcttgattgttttaacacagtacctaagactaatgctttctgtggacaccactgagctctgcctcaactccaccctctgcgaccggaggactatgcccctagtaactgctgtcggtgtggacgctgtgctggttctgttttctaaaggagcagaaggacaggtctctgagacaggatcgttgtccctacaggaggaacagtggccttgcttcttagacggtcttcactgtgtgttttaaaacaacaacaacaacaacaacaacataaaactcttttgacctgtaacttaaagatcataaacttcaggcaataatattttctgtgtaagcttttaaaattatttttggggatcatagcttgttttattttgtgctataaaattaacagtattaaatgacttatattcttagaataaaaaaaaaaaaaaaaaaaaa

SEQ ID NO: 112 - protein, 89 aa, Homo sapiens [sequence omitted]

SEQ ID NO: 113 - DNA, 2156 bp, Homo sapiens
  unsure (1353)
aagtgatctacctgcctgggcctcccaaggtgctgggattacgggtgtgagccaccgcgcccagcctattcttttttgtttgtgataatggtcatcctaatggacatgaggtagtgtcatgtggttttgatttgcatgtccctgataaataatgatgttgaccatctactcatgtgcttgttggctatttgcatggcgtgtttggagaaacgtctgttcaagggctttgccttttttttttgagacagartcttactccgttgccccargctggagtkcggtggtgaggggtgcactgcaacatccgccttccaggttcaagcgattcttgtgcctcagcctcccaaagagctgggattacaaaagtgcagtttgcccatttttaatcgattttgttcctgagttggagttttttgtatattcaggctgttaaccccttatgagatagatggtttgcacatagtctcttccattctataggatatcatttctgttaatagattcctttgctgtgcagaaactttttagtttgaggtcatcccatttgtctatttttactttcgttgcccttgctgttggtgtcatgttcaagaaatcattgccaagaccaatgtcgtgaagtctttccctttgttttcttctaagggttttacagtttcaagtctgtgtttgggtcttgcatcggttttgagttagtttttgtgtatgatgtaaggtaagggtctatctttatttgcaagtggatatccagttttcccagcgctgcatattgaagagaccatcctttccccattgtgcaagaagttcttgtcacccttgttgaaggtcatctgtctgtcattgtcatttctggccctgtgctgtcctgtcctgtcctgtcctgtcctgttctgttctgttggtctgtaggtctgtctttatgtcagcaccatactggctgttggactttttaattcttttcttgacagtggtaatttatttgcttctttttcttattagtccctttgcctactttaaataattaattttgttaatttttagttttctgttattttagttcattaatttcattgcttcctttatttatttatttattttttttgagatggagtcttgctctgtcactcaggctggagtgcagtggcacgatctcagctcactgcaacctccacctcccaggttcaagtgattctcctgtctcagtctcctgagtagctgggattacaggcacttgccaccatgcccggctaattttttgtattttttagtagagacggggtttcgctgtgttgcccgggctggtttcaaacttctgagctcaggcaatccacctgcctcggcctcccaaagtgntaggattacaggtgtgagccaccacgcctgacccattgctgccttaaatacacaaagcgcttgagttaataaagttacctgaaggattgaactttaatttctaacagcgtttggaggtgaggggactacttgtttttgctcatttttagtttttttttttttgcacttggggtcaaatggcatgtcatatgtgctgttacctgaaatatattgagggtttctttgttctatcatacmtggtcattttcataactgtcccacagacactggagaagcatgatgactccatggggtacagaatttagaacatccttgtcagattgagtctatggtgatgtgtcttaagtcgtcccttagtcttttttttcctaatcagtctgtcaaatttcagagaaccatgttaaaatcccctattattgtggttttgaaggttgtttccagtgtttttccttcatttaattcttcctctgtcgctgtgcgcctgcagattccaggctgcttgacatgggttcctttccatatgggagtgagccagcagacagccctacagatcgtacacacgttttccaaaactaacaatggaacaggcggcaaacctatgccaatatactagaaattgcagattaaatagatgaaatattctaaactggagtttacataatgaacataagagtaatcagagaatctgactcattttagatgtgtgtgtgtgtgtatatatatgtgtgtgtgtgtgaaaaacattgactataataaaaataatctcgagttcaaaaaaaaaaaaaaaaaaaaaaaaaaaaaaa

SEQ ID NO: 114 - protein, 94 aa, Homo sapiens
MVMCLKSSLSLFFPNQSVKFQRTMLKSPIIVVLKVVSSVFPSFNSSSVAVRLQIPGCLTWVPFHMGVSQQTALQIVHTFSKTNNGTGGKPMPIY

SEQ ID NO: 115 - DNA, 3941 bp, Homo sapiens
  unsure (2895)
cagacacagagatcagaattccaggaaatgatcttccagtgcgttctgggtcagttatggtgactgtaaataccgtcatcacagctggccctcaaaataacgcaataataacatatttacataatgacatattatgactgtaagtgcagtcagccccatctggggctgaggcgggggccctgctgtgcactctccccccagctatcccaccgggccaggggtgggcctcagggttgtgctgggagccgcagggcctgaaggggcctcggctgtacggggatgagactcgcaggggagagggcagaggccggtgacctggcgaggacttgcccaggagattggagctccttgcttctgcgccacgcggatgccccacgctggtctcagctgggttgttggctctgagtggtcatctcgttgctgccatattttcttgcttcattgaatttcactgtgctccagcctgggcaacacagccggactctgtttcaaaaaaaaaaattttttttttccaagataggatggtagagaaaatacctcctgccatgtcctgctatgaatacagctttgtatttctctctctagttttgtcagttttggcttttcagattttgaagcgtgtttgtgggctgaatcttgcccttatcacccatttctaggatgctttttgctccactcattctttgtcttgcttcacttgactttgaactgtatacttttttccatcgttttactttcagtatcttcatacatgtatgtttttgtacgcctctcttagaacagtgtatggttttgtaaaaattcagcctgtagcttttacctgcctccttcatgacctttataatccccttggttctcagcctgccactcacaggacttttccctgtgctgcgttccmagtgccccctccccgcccccacctgtgctttttgttggattagtagaattgcttttgtcattccattgttttcatatatttgtttgggacattttacttttttctgttaacgcttaccctagaaattagaaatgacaccacgtattcttagcgaagtccagttttcagcattttgtccttattggacaatagcaaggatattagaacgtgttggttccgcgtgcttccgtcttgagttatgtgctgctattgtcggatattttgtcttagatgtacgtactttcctgttcattgtggtatgtgtaatttgcgttactttgaattttccacgtttttactttctttgtctctcatcacttactgcttttgggaccccccccatcggggttcacattccctctccctagagcacactcccttggatttcctcgagtggggtctgctgcggtgaagctttcccattttatgtgcagattattttcagagggtatatagaattcaggcagctgtttcgttgtagcacattaaaaatattttcccacttcctccttgcttctgttgttgcttttgagtgttacctctgagtctgcctgtgctccctggaaacggcccgggtttcccaccccctgcccaggtttgctccttccgtggtttttctgtcattatcacgctcacgtgtttccctcggtcaccccctctgcaattttcacacgtcttttccctctctctttgcttcattacctttggcccgcctgccagctgctgattctctctgaagatgtctctaaatgacttttaactgtgatttgtggaattcttattgtggagttttgcgtcttttcaggtgtaggttttttgtctcgcgtgtttccacgtctgcttgtagcgctttccgcttcgccgttccctgcggcccttccttccgtgcccggtgttcatcctcttgaatgctcttttcctgcctgtttggctgggtgtgtctgagttgcaacctgagcgggtttctttgtcttcttacttgtctggtattgtgttctctcgggacgttgcgtttgaggggtcgcacctcagagcaagccgaggtctgggctaagcctgtgctttggcaggcaggaccttagtttgccttttctgggcacctgaggagagggtagcagcagcctggggtctccttgactcacggtcagcagtgagggtttcctggcctgttgggtggctggagcttggctgcattccccactgagagagggaggtgcgcaccttctcctccctggagtggccttccaggtgccctctcagagctgctcatcagggctgtgcctttgtcagcaccaagcctcagcccttgtccctgctgccactgaaggctcaaaacaacactgcacagccttgtgtgtcctctgtgtgtcggcagtttcccccggctctgcagcagcccaggccaggtagcctctggagggagtggtggaggagcacgggcatcctggccgccgctgtgttggggacagaccctggggcctggaaagggaggtgaggccccgtgggggctgctgcaccacaggcaagagagcaagagacagcagaggccggccaggtggtggcacagccgctagggaccaggccggcctgtggaggtattgggatggggaccagcggacttgctggcagaggggcctcagggctgcaggcttcttggactgagccactgggaggacggagttgaccttctttgagacagaaaaagtgtgcatcccggggctgcctgtgaaagctcatctctaaagtgtgtgttgttcttccagccacccctttgctgtgaagttgcttgcgctctgtaagaaagaaatcaagaattcaaaagatatccagaagctcctgtcaggcatcgcagtgtgagtttcaagtgctactggccttagacggaatggcagggcgcagcctcccttggctgagggcaggagtccacggctccaggcgggagaggagcagttagtgtnactcctcaagctaacctaagatcgtgcattccaatgttcaaagcagtcgcaatgggaggtgaggcagcccaggtgctggtggagggagttcccgcgggaacaggcgagctctgcctctgctgccctcgcgctctgccctggcgggaggggaggctccggaaaggagctgcgtggtcaggggctgcctccccgattctcctgtgtgccctgggggtcgctgttgagtgccttgctctgcggcgctcaggtggacactgggcaggtgcgccagccagcgataggcaccttggctgctctgtggctccttgaggtgggggtcctcatggcagggcgagcggccctgcaggagatcctctgtgaggcgtcctcacttcccacagtgactttccaagtgcgacactcgcgtgtgtaggcacagtgcagatgtgcgcacacacacacctccggcttggggccccaggcccgcactgtgctcacggatctgctctgcccaggttctgcgggatggtgcagttccccggcgaacgtgaggaggcaggccctcctgcagctgtgtctgctcctctgccaccgtttccsgctgatccggaagaccacggccagccaggtgtacgagacattgctcacctacagtgacktcgtgggcgcggatgtgctggacgaggtggtgactgtgctcagtgacactgsgtggacgcagagcttgcagtggtgagagagcagcgcaaccgtctgtgtgaccttctgggcgtacccaggccccagytggtgccccagcctggtgcctgctgaagccagtcctggagcccatacctcacccctgcctggtgaggatgtcttgttcctgagggaggccggtgtggaaagcctcgcacagtggtgcctccagctgttgaagggtagcgctggcccttggaggctggcactagctgacagcttttcctctctgcacctgcgctctggtgacttggggtggacgcctctgccttcacttgaacacaaatgtgcttcctataaaatcatgtaccaagaaaaaaaaaaaaaaa

SEQ ID NO: 116 - protein, 70 aa, Homo sapiens
MCCYCRIFCLRCTYFPVHCGMCNLRYFEFSTFLLSLSLITYCFWDPPHRGSHSLSLEHTPLDFLEWGLLR

SEQ ID NO: 117 - DNA, 1779 bp, Homo sapiens
ccaagttccaggtctagaattcaaattactaatttactgcttctctctctctaagcctcagctccctgatctagaccatgagatttacagtaggagagtaccatgtttatccccaaatacttaacagctagggttttcccagactgaataataataataacttttttaaaattcagaaggtatcttcaagttcttggcttgcttcttgtacattcaatatcaaagaagagaaaacacactatctgagagtacttcccatgcacctaataagtgccaaagccacctggtgctagagcccttcaccaaaatgagcatcagccttgctttcagaaagcagggaccacatatatatgatttaaaaaaaatctgcgatcaacttttctctaaaaaacccaaatatgctggggtacagaaagatcaatgcaaaagcaaaacatcctgtgcctgtcctagaggtccccagaggcaggatgccccgactcagaaagaaactcctaagctggcctggccaaagggaggaagaacccagggtgggtgtcgtaactcatctaaaaataacgatgtcatcaggcagatgtgccattgtgctggggctgggtgggtgtggcaggcccaccttgggtatgcaaagctctgacagtgtttcacttgctaccctcggtctgcttaccacactcccagttctgctgaccttacgggaaggctcatgctgggttgactcacggcaggcctagagcactgtgagggatgtgtgaggacaagggtcacaccccagggtggcatttccaagccccatgcctctggccatatcccataggggctctaggcctctgttttcccatctttaaaataattgggggcaatacctcctatgatctttctgagaattaatagagatttcatggcaattgcttagccctgcccagcagagatagcaaataatcaatcagctccctttctcctctgtctcttgggtgttttctactcctggaaccccagagcaagagaggaccctgaaacatggcctacatccaattctttcattttgcatttgaggaaatcgaggcacatggctgcggttctactcttaccaacccatatcaggtcattgctctaacgaggcttaaggagcaataacccgcctttcacgtggttcttacggatacccagaaagatgactcagcttctccagatttctgagaagactaagcataagtcagagagagtatagacaaaggaaaagggggcataactgcaaggaccccctcaaatgtgtgctgtggcagcattggtgggacaggggctgaaagagcaaaacagtagggatcacatcttggagagtactcgggaaggagtccaaaaacgaccatggatcctggagctacaggttgcaaccaaactacaatcattccatttggcctcaggatgtggaagcaccccaaatgtgtttgcctcaaaaagcaaagaggatgaggcccggcatggtagctcaggcctgtaatcccagcactttgggaggccgaggtgggcggatcacttgagtccaggagttcgagatcagcctgggcaatgtagcaacaccgcacctctacaaaaaataaaagaattaactgggcgtggtggcgcatgcctgtagtcccagctactctggaggctgaggtgggaggatcccttgagcccaggagatggaggttgcagtgagctgagatggcaccactgcactccagtctgggtgacagagcaagacccagactcaaaaaaaaaaaaaaaaaaaaaaaa

SEQ ID NO: 118 - protein, 109 aa, Homo sapiens
MSISLAFRKQGPHIYDLKKICDQLFSKKPKYAGVQKDQCKSKTSCACPRGPQRQDAPTQKETPKLAWPKGGRTQGGCRNSSKNNDVIRQMCHCAGAGWVWQAHLGYAKL

SEQ ID NO: 119 - DNA, 1170 bp, Homo sapiens
agccgcgcggctgcgggggcgcaaatagggtcactgggccgcttggcggtgtcgttgcggtaccaggtccgcgtgaggggttcgggggttctgggcaggcacaatggcgtctcgagcaggcccgcgagcggccggcaccgacggcagcgactttcagcaccgggagcgcgtcgccatgcactaccagatgagtgtgaccctcaagtatgaaatcaagaagctgatctacgtacatctggtcatatggctgctgctggttgctaagatgagcgtgggacacctgaggctcttgtcacatgatcaggtggccatgccctatcagtgggaatacccgtatttgctgagcattttgccctctctcttgggccttctctcctttccccgcaacaacattagctacctggtgctctccatgatcagcatgggactcttttccatcgctccactcatttatggcagcatggagatgttccctgctgcacagcagctctaccgccatggcaaggcctaccgtttcctctttggtttttctgccgtttccatcatgtacctggtgttggtgttggcagtgcaagtgcatgcctggcagttgtactacagcaagaagctcctagactcttggttcaccagcacacaggagaagaagcataaatgaagcctctttggggtgaagcctggacatcccatcgaatgaaaggacactagtacagcggttccaaaatcccttctggtgattttagcagctgtgatgttggtacctggtgcagaccaggccaaagttctggaaagctccttttgccatctgctgaggtggcaaaactataatttattcctggttggctagaactgggtgaccgacagctatgaaacaaatttcagctgtttgaagttgaactttgaggtttttctttaagaatgagcttcgtccttgcctctactcggtcattctccccatttccatccattaccccttagccattgagactaaaggaaatagggaataaatcaaattacttcatctctaggtcacgggtcaggaaacatttgggcagctgctcccttggcagctgtggtctcctctgcaaagcattttaattaaaaacctcaataaagatggccctgcccacaaaaaaaaaaaaaaaaaaaaaaaaaaaaaaaaaaaaaaaaaaa

SEQ ID NO: 120 - protein, 183 aa, Homo sapiens
MASRAGPRAAGTDGSDFQHRERVAMHYQMSVTLKYEIKKLIYVHLVIWLLLVAKMSVGHLRLLSHDQVAMPYQWEYPYLLSILPSLLGLLSFPRNNISYLVLSMISMGLFSIAPLIYGSMEMFPAAQQLYRHGKAYRFLFGFSAVSIMYLVLVLAVQVHAWQLYYSKKLLDSWFTSTQEKKHK

SEQ ID NO: 121 - DNA, 1127 bp, Homo sapiens
ctcgccgcagaagtatctccgaatggagccatcccccttcggcgacgtctcctcccgcctcaccacagaacaaattctgtacaacataaaacaagagtataaacgaatgcagaagagaagacatttagaaacgagtttccaacagacagatccgtgttgtacttctgatgcacagccacatgcatttctcctcagtggaccagcttcaccagggacttcatctgcagcatcctcaccattaaaaaaagaacagcccttatttactctacggcaggttgggatgatctgtgaacgtttgttgaaagaacgtgaagagaaagttcgagaagaatatgaagaaatattgaacacaaaacttgcagaacaatatgatgcgtttgtgaagtttacgcatgatcaaataatgcgacgatatggagaacagcctgctagctatgtttcatgaatcacgtatcctgcatttgtgggctgccttgttccttgttgagttgttgcaagaggtcccaattatgacatgcagcaatgccaataccccttctgtgaatacaggttatttcaagctttcgtcagtggcaaccactcttaggcagcagcaactggttttggaaatttccctgatgtcagtaccacctggatgtggacctttgctacctgtattaataccagtggcctcattttgctgtatcattacaatttggcttcttatattaatgtttgaaaaggattaaagctggtattctagaacatgcccttcactggttgtgtaaataaaactgtagaatgacacttcagatgaagttagtgtgattttaattgtgcactacaaccgagctgtaaccagttactaattttagaatgtaatcccaggacaatattaagcaaatagcctgcagtgcttcctgtgaaatagtgaaggaggagggcatttctgtattccaggacttcttggggtttcagaatgggtttgtatgatttttttttttttgtagttttatttattctatcagtctttttaacaaatgtttattgctgcatttttttttttccagtgtatcattgttttactgcccttgtagtactggaatttagttggaagaataaaacatttacttctaaaaaaaaaaaaa

SEQ ID NO: 122 - protein, 140 aa, Homo sapiens
MEPSPFGDVSSRLTTEQILYNIKQEYKRMQKRRHLETSFQQTDPCCTSDAQPHAFLLSGPASPGTSSAASSPLKKEQPLFTLRQVGMICERLLKEREEKVREEYEEILNTKLAEQYDAFVKFTHDQIMRRYGEQPASYVS

SEQ ID NO: 123 - DNA, 806 bp, Homo sapiens
gtgtatcttcagaggcagcaggggccagtgtgccacatcttgccccagtcctgaaaggatagatggtatttggcctgtgacccttggctgaggagccatggtccggctctgccaggccctgctgctgttagtggccactgtggcccttgcatccagaagattccaagcctggggctcaacaaargtggtgaggacattccaagatatccctcaaaactacgtctatgtkcarcakgcactctggttcgccatagaaggagtataacaaggccagctttagtataacaagttcagctttagggtgctgaaggttctgaagagccasgarcaggtgacagatagtttggagtactatattgaggtcaaaattgcccgaacartttgcaagaaaatttcagaagatgaaaactgtgcatttcaagaggatcccaaaatgcaaaaggtggttttttgtayttttattgttgcatctaaaccatggaaatttgaactcaccatgytgraaacaatgcaaagatatgtagttatcttctmgtgtgttctgccacactcatttccattttaaagaagaagcaaagacayttgcaagaaytagaacaacacagttaacccattaacttcatttgtttggcctttttgcatttttgtgtgttcttcatgggctgatgttgaaaatccatgatgtgttttgacagcattgcatagcctattcttgctggatacttcccctactagctgggataatctgytgcaataaatggaagtggtttcttacacstcaaaaaaaaaaaaaaaaaaaaaaaaaa

SEQ ID NO: 124 - protein, 55 aa, Homo sapiens
  UNSURE (46)
MVRLCQALLLLVATVALASRRFQAWGSTKVVRTFQDIPQNYVYVQXALWFAIEGV

SEQ ID NO: 125 - DNA, 1783 bp, Homo sapiens
tccccaccccccttatgtctcagccgaacctaccctaatccagcccacgccacaatggtgggacaggttccccagtccctatgtggtcttatttttacccttgcactccctgtagaccatcaattctacaccctaattacaaaatcatatccacctctgcctggcagaaggtgttatgcttttctggctcgcctaccatccacacatccctacacctcaccaccggatcctcttttctttccttccatccaattcctggcttccccgctgccaactctgctctctatgtctccagtttaaaggtgccccctggaaaaaatgtaacaattccctcacctgtgactggtacctgacagccaccacaccggggcagcaatggctaacggttgacaaagacaatttctttctctctccaaaaccaaacagccttcatcaactccctagccaagactccctatcaggcccttacaggtgccgctctggctggcagttacccmatttgggaaaacgaaaataccctatcatggctacctaccttcacctacaacttctgcctgtccacccccagtctcttctttttgtgtgatacaaactgatatctttgcctaccagccaactggtcaggaacttgcaccctggtctttcaggctccaaccatcaacatcctaccccctaaccaaactattctaatttctgtagaagcctctatctcctcttcacccataagaaataaatgggctctacatctcatcaccctgctaacaggattaggcatcactgctgcacttggcactggaatagcaggcataaccacctcaatcacctcataccaaacactattcacaaccctttctaacaccgtagaagatatgcacacttccattaccagtctccaacgacaattagacttcctcgtgggagtcatccttcaaaactggagagtcctggacctcctaaccactgagaaagggggtacctgcatatacctccaggaagaatgctgtttctgtgttaatgaatctggcattgttcatatcgcagttcgtaggcttcatgacagggctgcagagctttgacatcaagtcgctgactcctggtggcaaggatcatcccttctaagatggataccctgggttgcccccttcctaggacccctgatcttcctcttcctgttactaatgattgggccatgcatatttaaccttgtatcccgcttcatttcccaaaggctgaattgttttatccaggcaagcatgcaaaaacacattgataatatatttcacctttgccacgtctaataccagagcctacgaggaaaccattcggaagctccagaacccaggccctaatcacaacgcccctatccagcaggaagcagccagatgatyaamgacgccctttttcctttttatactaaagtaagaaataagaatgttagcccaaactgcaytattttgcagacccctaccattttacaaactggtcagagtggaaaattccaccagggcctgagctgtgagaaacatcctgtcaggcaggtcccaggcctaacccctggstgcactaaattccttcattatcagcagccaaacacaccgcccccaccccattttcacaacaatcccagacctctcctgcccgggactgtaactggtccagcctgtaagcgggaagggggctctggcactagstggtaccccctctccgcaggtctttctcccaataaatctgtgttgcccttgraaaaaaaaaaaaaaaaaaaaaaaaaaaaaaaaaaaaaaaa

SEQ ID NO: 126 - protein, 136 aa, Homo sapiens
  UNSURE (108)
MLFWLAYHPHIPTPHHRILFSFLPSNSWLPRCQLCSLCLQFKGAPWKKCNNSLTCDWYLTATTPGQQWLTVDKDNFFLSPKPNSLHQLPSQDSLSGPYRCRSGWQLPXLGKRKYPIMATYLHLQLLPVHPQSLLFV

SEQ ID NO: 127 - DNA, 3149 bp, Homo sapiens
ggtctttaacgtgagcccgctgcaggtgtgcggcccagtccgagacagcagatgaggagactgtccttcctgtttcgcagatgaggaaactgaggcttagagaagtttggcaaattggctaagttcctacagctaccacagcagaaagtgctgggcagtagagagctgccccctccagaagatgatcagctgcactccagtgcccccagatcctcgtggaaggaacggatccttaaagcaaaggtggtgacggtgtctcaggaggcagartgggatcaaatcgagcccttgcttagaagtgaattagaagattttccagtacttggaattgactgtgagtgggtaaatttggaaggcaaagcctgccctctgtcacttctacaaatggcctccccaagtggcctgtgtgtcttggttcgcctgcccaagctaatctgtggaggaaaaacactaccaagaacgttattggatattttggcagatggcaccattttgaaagttggagtgggatgctcagaagatgccagcaagcttctgcaggattatggcctcgttgttagggggtgcctggacctccgatacctagccatgcggcagagaaacaatttgctctgtaatgggcttagcctgaagtccctcgctgagactgttttgaactttccccttgacaagtcccttctacttcgttgcagcaactgggatgctgagactctcacagaggaccaggtaatttatgctgccagggatgcccagatttcagtggctctctttcttcatcttcttggataccctttctctaggaattcacctggagaaaaaaaacgatgaccacagtagctggagaaaagtcttggaaaaatgccagggtgtggtcgacatcccatttcgaagcaaaggaatgagcagattgggagaagaggttaatggggaagcaacagaatctcagcagaagccaagaaataagaagtctaagatggatgggatggtgccaggcaaccaccaagggagagaccccagaaaacataaaagaaagcctctgggggtgggctattctgccagaaaatcacctctttatgataactgctttctccatgctcctgatggacagcccctctgcacttgtgatagaagaaaagctcagtggtacctggacaaaggcattggtgagctggtgagtgaagagccctttgtggtgaagctgcggtttgaacctgcaggaaggcccgaatctcctggagactattacttgatggttaaagagaacctgtgtgtagtgtgtggcaagagagactcctacattcggaagaacgtgattccacatgagtaccggaagcacttccccatcgagatgaaggaccacaactcccacgatgtgctgctgctctgcacctcctgccatgccatttccaactactatgacaaccatctgaagcagcagctggccaaggagttccaggcccccatcggctctgaggagggcttgcgcctgctggaagatcctgagcgccggcaggtgcgttctggggccagggccctgctcaacgcggagagcctgcctactcatcgaaaggaggagctgctgcaagcactcagagagttttataacacagacgtggtcacagaggagatgcttcaagaggctgccagcctggagaccagaatctccaatgaaaactatgttcctcacgggctgaaggtggtgcagtgtcacagccagggtggcctgcgctccctcatgcagctggagagccgctggcgtcagcacttcctggactccatgcagcccaagcacctgccccagcagtggtcagtggaccacaaccatcagaagctgctccggaaattcggggaagatcttcccatccagctgtcttgatagctgctttcctcccagttaggacaagtgggaagctggagccaaggttgaagagtcacctcttcccattttagtacatcattaattgtcaaagcctgtgtgacacaactcagaatactaacctagactaatcccaggatgcttctgctggagcaaagatattgtttgaaggagagtttatggttttggattttaaacgggcagggtcttttttcctctcatttttgtggacaagagaggccttcgcctttatttttactctccctcttctgctgtccctgtgcagaggaaaaatgaagaattctcccagaagtgacttgtcaagacttaaaaaaaatgtttttaatgcatttcttccttgtctagtgcctcggtttatctctaacaggggctgtccagtatatcggtcctgttaggaggggagaaaaagttcttccaaaggctggagaagtgaacaaggagtcaaatttattttcccaattcaacttcataattatcatttctttggcttcatgctctcccgtaactcatgtggttgggatccatcccatctgggtcacttcagtctacttcacgtacttgaaaaggctttcctttacacttccaggaccaaacagcaacttcctgccacacacttccaccctatcactgggagaaatccttttctggacatgagcctttgacctgggtggggcagaaagaaccacaaactccatctcccaatagaactttgaaattcactcagcttttcctttcatgctgtttgttgcctgcttgttgcactcctcctgccccagaactgcaagatttttagcttcacccctttctgagagtaatgttatcttttatcagaatcagtatcagttcccctgtattctgtgcttcatcgaatttgcaagactgacctcttttaagcatttaattcactcccagagtcatctggtcaggttgcaatatgaggacttctctgtctcctctgaagcctgggacactgagcttacttaatacattagatgttcaaaagaggagcgttgtttcatctttcaaaatgttaggccattactttgagtataaaatcgacttattaatgattagtaatttttctaaagtattgggaaaactttcttattttataagatcttaacaagcttaaaaaagaattttatgaccagaatccaacaagagctctattttggaattgtgcccaagttggtgatgtttactctaaaattaataataaaactacttgtaagcaaaaaaaaaaaaaaaaaaa

SEQ ID NO: 128 - protein, 380 aa, Homo sapiens
MLPGMPRFQWLSFFIFLDTLSLGIHLEKKNDDHSSWRKVLEKCQGVVDIPFRSKGMSRLGEEVNGEATESQQKPRNKKSKMDGMVPGNHQGRDPRKHKRKPLGVGYSARKSPLYDNCFLHAPDGQPLCTCDRRKAQWYLDKGIGELVSEEPFVVKLRFEPAGRPESPGDYYLMVKENLCVVCGKRDSYIRKNVIPHEYRKHFPIEMKDHNSHDVLLLCTSCHAISNYYDNHLKQQLAKEFQAPIGSEEGLRLLEDPERRQVRSGARALLNAESLPTHRKEELLQALREFYNTDVVTEEMLQEAASLETRISNENYVPHGLKVVQCHSQGGLRSLMQLESRWRQHFLDSMQPKHLPQQWSVDHNHQKLLRKFGEDLPIQLS

SEQ ID NO: 129 - DNA, 1861 bp, Homo sapiens
agagccaggggggtcgcgtagtgtcatgaccagggcgggagatcacaaccgccagagaggatgctgtggatccttggcggactacctgacctctgcaaaattccttctctaccttggtcattctctctctacttggggagatcggatgtggcactttgcggtgtctgtgtttctggtagagctctatggaaacagcctccttttgacagcagtctacgggctggtggtggcagggtctgttctggtcctgggagccatcatcggtgactgggtggacaagaatgctagacttaaagtggcccagacctcgctggtggtacagaatgtttcagtcatcctgtgtggaatcatcctgatgatggttttcttacataaacatgagcttctgaccatgtaccatggatgggttctcacttcctgctatatcctgatcatcactattgcaaatattgcaaatttggccagtactgctactgcaatcacaatccaaagggattggattgttgttgttgcaggagaagacagaagcaaactagcaaatatgaatgccacaatacgaaggattgaccagttaaccaacatcttagcccccatggctgttggccagattatgacatttggctccccartcatcggctgtggctttatttcgggatggaacttggtatccatgtgcgtggagtacgttctgctctggaaggtttaccagaaaaccccagctctagctgtgaaagctggtcttaaagaagaggaaactgaattgaaacagctgaatttacacaaagatactgagccaaaacccctggagggaactcatctaatgggtgtgaaagactctaacatccatgagcttgaacatgagcaagagcctacttgtgcctcccagatggctgagcccttccgtaccttccgagatggatgggtctcctactacaaccagcctgtgtttctggctggcatgggtcttgctttcctttatatgactgtcctgggctttgactgcatcaccacagggtacgcctacactcagggactgagtggttccatcctcagtattttgatgggagcatcagctataactggaataatgggaactgtagcttttacttggctacgtcgaaaatgtggtttggttcggacaggtctgatctcaggattggcacagctttcctgtttgatcttgtgtgtgatctctgtattcatgcctggaagccccctggacttgtccgtttctccttttgaagatatccgatcaaggttcattcaaggagagtcaattacacctaccaagatacctgaaattacaactgaaatatacatgtctaatgggtctaattctgctaatattgtcccggagacaagtcctgaatctgtgcccataatctctgtcagtctgctgtttgcaggcgtcattgctgctagaatcggtctttggtcctttgatttaactgtgacacagttgctgcaagaaaatgtaattgaatctgaaagaggcattataaatggtgtacagaactccatgaactatcttcttratcttctgcatttcatcatggtcatcctggctccaaatcctgaagcttttggcttgctcgtattgatttcagtctcctttgtggcaatgggccacattatgtatttccgatttgcccaaaatactctgggaaacaagctctttgcttgcggtcctgatgcaaaagaagttaggaaggaaaatcaagcaaatacatctgttgtttgagacagtttaactgttgctatcctgttactagattatatagagcacatgtgcttattttgtactgcagaattccaataaatggctgggtgttttgctctgtttttaaaaaaaaaaaaaaaa

SEQ ID NO: 130 - protein, 571 aa, Homo sapiens
  UNSURE (202)
  UNSURE (504)
MTRAGDHNRQRGCCGSLADYLTSAKFLLYLGHSLSTWGDRMWHFAVSVFLVELYGNSLLLTAVYGLVVAGSVLVLGAIIGDWVDKNARLKVAQTSLVVQNVSVILCGIILMMVFLHKHELLTMYHGWVLTSCYILIITIANIANLASTATAITIQRDWIVVVAGEDRSKLANMNATIRRIDQLTNILAPMAVGQIMTFGSPXIGCGFISGWNLVSMCVEYVLLWKVYQKTPALAVKAGLKEEETELKQLNLHKDTEPKPLEGTHLMGVKDSNIHELEHEQEPTCASQMAEPFRTFRDGWVSYYNQPVFLAGMGLAFLYMTVLGFDCITTGYAYTQGLSGSILSILMGASAITGIMGTVAFTWLRRKCGLVRTGLISGLAQLSCLILCVISVFMPGSPLDLSVSPFEDIRSRFIQGESITPTKIPEITTEIYMSNGSNSANIVPETSPESVPIISVSLLFAGVIAARIGLWSFDLTVTQLLQENVIESERGIINGVQNSMNYLLXLLHFIMVILAPNPEAFGLLVLISVSFVAMGHIMYFRFAQNTLGNKLFACGPDAKEVRKENQANTSVV

SEQ ID NO: 131 - DNA, 2157 bp, Homo sapiens
ctctctttaatatcttcacctctaccatgtgtctttcttttaatatagttataattttccaaccacgtagatcaatatttactcatcatgaccataaaatgcagtttagccatatagaaaactatgattacttttctttataatttcccttcagttaatacttattttattttctgtttttatcatctagtcaactcgcaaacttccagcatttgtctaaatctactcaatatattccagtacatcagataatatatcagtttcatcctcctgaaaaactcttttccagtgtatcctgacctgctctaattttgacttgatgctttctgtatctggtgcacagctgttaccttggaatcttcccttcatcattattcagagtgtttctgtagtttttctcttgcattggattttgtgcttcctgaatccctctctctctttttttttttttttttacttggcttactccttgctttgatggatctcaggctccagtagcttccttggaaagagtgtttggaagttgcttctgcaggaagcctttttggtggcatggtcctcaagaagttcctaaaaggttgatgaaaagcccagaaccttgatgacagattgtctggttataaagcattttttacgtaaaatcatcatggtgcaccctaaggtcagatttcatttcagtgtaaaggtaaatggaatcctctccacagagatctttggggtggagaatgaacccactttgaaccttgggaatggaattgctcttttggtcgactcccagcattatgtgagtagaccaaattttggtacaattgaatcacactgcagcagaattcaccctgtgctaggacatccagtaatgcttttcatccctgaagacgtggctggcatggacttgttgggagaactgatactgactccagcagctgcactgtgccccagcccaaaggtttcttccaaccagcttaacaggatttcttcagtttccatatttctatatggacctttgggtctgcctctgatattgtcaacttgggagcagccgatgactactttcttcaaagatacctcttctttagttgactggaaaataccatttgtgtatgatacccaatttggatctcaatttggatagagatttggtgcttccagatgtgagttatcaggtggaatccagtgaggaggatcagtctcagactatggatcctcaaggacaaactctgctgctttttctctttgtggatttccacagtgcatttccagtccagcaaatggaaatctggggagtctatactttgctcacaactcatctcaatgccatccttgtggagagccacagtgtagtgcaaggttccatccaattcactgtggacaaggtcttggagcaacatcaccaggctgccaaggctcagcagaaactacaggcctcactctcagtggctgtgaactccatcatgagtattctgactggaagcactaggagcagcttccgaaagatgtgtctccagacccttcaagcagctgacacacaagagttcaggaccaaactgcacaaagtatttcgtgagatcacccaacaccaatttcttcaccactgctcatgtgaggtgaagcagctaaccctagaaaaaaaggactcagcccagggcactgaggacgcacctgataacagcagcctggagctcctagcagtgcttaaacagccttcccagcccacagcagcaggggtacagcagctctcacattcagtcactagcagagatgccagataccagcgggcaagcagaaaacaagaggctcaagaggggcagcccccgcatagaggagatgcgagctctgcgctctgccagggccccgagcccgtcagaggccgccccgcgccgcccggaagccaccgcggcccccctcactcytagaggaagggagcaccgcgaggctcacggcagggccctggcgccgggcagggcgagcctcggaagccgcctggaggacgtcctgtggctgcaggaggtctccaacctgtcagagtggctgagtcccagccctgggccctgagccgggtccccttccgcaagcgcccaccgatccggaggctgcgggcagccgttatcccgtggtttaataaagctgccgcgcgctcaccaaaaaaaaaaaaaaaaa

SEQ ID NO: 132 - protein, 270 aa, Homo sapiens
MIPNLDLNLDRDLVLPDVSYQVESSEEDQSQTMDPQGQTLLLFLFVDFHSAFPVQQMEIWGVYTLLTTHLNAILVESHSVVQGSIQFTVDKVLEQHHQAAKAQQKLQASLSVAVNSIMSILTGSTRSSFRKMCLQTLQAADTQEFRTKLHKVFREITQHQFLHHCSCEVKQLTLEKKDSAQGTEDAPDNSSLELLAVLKQPSQPTAAGVQQLSHSVTSRDARYQRASRKQEAQEGQPPHRGDASSALCQGPEPVRGRPAPPGSHRGPPHS

SEQ ID NO: 133 - DNA, 1607 bp, Homo sapiens
gtgaacttcactactggaaagcaacaaaggcagtcggcataaaaatgggttctctcagcacagctaacgttgaattttgccttgatgtgttcaaagagctgaacagtaacaacataggagataacatcttcttttcttcgctgagtctgctttatgctctaagcatggtcctccttggtgccaggggagagactgcagagcaattggagaaggtgcttcattttagtcatactgtagactcattaaaaccagggttcaaggactcacctaagtgcagccaagctggaagaattcattccgagtttggtgtctaattctctcaaatcaaccagccagactctaactgtaccctcagcattgccaacaggctctacgggacaaagacgatggcatttcatcaggaaaagtcgcaaatctctttggaaagagcacaattgacccttcatctgtaatggtcctggtgaataccatatatttcaaaggacaatggcaaaataaatttcaagtaagagagacagttaaaagtccttttcagctaagtgagggtaaaaatgtaactgtggaaatgatgtatcaaattggaacatttaaactggcctttgtaaaggagccgcagatgcaagttcttgagctgccctacgttaacaacaaattaagcatgattattctgcttccagtaggcatagctaatctgaaacagatagaaaagcagctgaattcggggacgtttcatgagtggacaagctcttctaacatgatggaaagagaagttgaagtacacctccccagattcaaacttgaaattaagtatgagctaaattccctgttaaaacctctaggggtgacagatctcttcaaccaggtcaaagctgatctttctggaatgtcaccaaccaagggcctatatttatcaaaagccatccacaagtcatacctggatgtcagcgaagagggcacggaggcagcagcagccactggggacagcatcgctgtaaaaagcctaccaatgagagctcagttcaaggcgaaccaccccttcctgttctttataaggcacactcataccaacacgatcctattctgtggcaagcttgcctctccctaatcagatggggttgagtaaggctcagagttgcagatgaggtgcagagacaatcctgtgactttcccacggccaaaaagctgttcacacctcacacacctctgtgcctcagtttgctcatctgcaaaataggtctaggatttcttccaaccatttcatgagttgtgaagctaaggctttgttaatcatggaaaaaggtagacttatgcagaaagcctttctggctttcttatctgtggtgtctcatttgagtgctgtccagtgacatgatcaagtcaatgagtaaaattttaagggattagattttcttgacttgtakgtatctgtgagatcttgaataagtgacctgacatctctgcttaaagaaaaccagctgaagggcttcaactttgcttggatttttaaatattttccttgcatatgtaaatagaatgtggtgagttttagttcaaaattctctgttgagaataataaatgcatgaaataccttaaaaaaaaaaaaaaa

SEQ ID NO: 134 - protein, 217 aa, Homo sapiens
MVLVNTIYFKGQWQNKFQVRETVKSPFQLSEGKNVTVEMMYQIGTFKLAFVKEPQMQVLELPYVNNKLSMIILLPVGIANLKQIEKQLNSGTFHEWTSSSNMMEREVEVHLPRFKLEIKYELNSLLKPLGVTDLFNQVKADLSGMSPTKGLYLSKAIHKSYLDVSEEGTEAAAATGDSIAVKSLPMRAQFKANHPFLFFIRHTHTNTILFCGKLASP

SEQ ID NO: 135 - DNA, 1537 bp, Homo sapiens
gtaggatttggggatgtggatatttaagacaatttcttttttcttttggtttaataggggcgggtatagggaccaactgggaccgagtgcccagggggccgagcacggtcatgctggccggcctgcatgcatgcgtgtgccgggctgggctgggcggccggcggtcgtggggcagggttgggggtctgtgctcagctgataactgccatgcactgtactgcacacgtccctagagcctaccgggacccgacgcttttcagggcatttctccctccagccagggcccaactcccacctgcctgggcgaatctcctccaaggaagtcccaggaggatggggaccaggaaggctgtggacccccatctccagggggccttcccagcctgatccctgtcctccaagttctggaggaggccgctgtagggtctggctgagcttcccacccactttccctggtcccaatcctttcttgtcctatacccagctggggttgctgccctgaacgaactgcgtgtggggccggcacatcctagcaggcagcccctggcgcctgctgcctcagggatgctccaaccaccctcgttctcctcgcagtggccctggctcccacctcccgccccagcctgccgtggggcccgtcagcctggtcccacccccatggagaacccaaagtcttactgtatataactccaggtgacgtttctatatttatagcagtgttgaaaacccacgtgttttacacagaaccaccctctccaacccctcccttcccgaccccaacaaaacgttttcaaaccccttacagttcctggggcaggcggaaacaggctcacagattgtgtgtcggctgcagcagtgattccaacaagcagctattgggggggaaacacagcatttaaaaagatcatcattaaaaaacaagatttatacaacaattacttaggatgtttgtgatctgccgaccttgctatagatgccatgttaccaatgatttcctgtggtgggggcttgccattgtttactctcttatttaccaacttctggcctaggcatgacagtgggcaccttcccccagccctggctgggcccagcgcctgtgttytgtgttagaaaggttttatatatatataaaattacatatatakgtagaaatatatgtaattttgggggccctgttccttgcacattttacagttacctcatttttcccatgtatgtatttgagaaaatgctaatatatagagaaaaaaatggttcttaaaacttaaatgtgtggttttttccattccatgggattcacattggtttgtagcatttaacataactagtatgttgtattatatatatgtgtatactgattgaaatttttaacagatttgtactttttttaaaatgaaagttgctagttctgcttgaccaagtagtgcaatcattattttttttaatattgttgctgatttcagagggatattcactaataaatgtatgatgtatacccacgraaaaaaaaaaaaaaaaaaaaaaaaaaaaaaaaaaa

SEQ ID NO: 136 - protein, 86 aa, Homo sapiens
MHACAGLGWAAGGRGAGLGVCAQLITAMHCTAHVPRAYRDPTLFRAFLPPARAQLPPAWANLLQGSPRRMGTRKAVDPHLQGAFPA

SEQ ID NO: 137 - DNA, 1302 bp, Homo sapiens
cttcatggcctacacacaccaccttacccctctgctggcaagaggggacctgattcatcctcacgctaaacactcattctacccaactgattgagacagaacagaagataaactgaaacttctctgccttcccgctgcaagagtgaatgagcgatccctctcaactgactcaaaatgtttgcctcacccaggagatggagctctcgaaggccttctctggccagcggacactcctatctgccatcctcagcatgctatcactcagcttctccacaacatccctgctcagcaactactggtttgtgggcacacagaaggtgcccaagcccctgtgcgagaaaggtctggcagccaagtgctttgacatgccagtgtccctggatggagataccaacacatccacccaggaggtggtacaatacaactgggagactggggatgaccggttctccttccggagcttccggagtggcatgtggctatcctgtgaggaaactgtggaagaaccaggggagaggtgccgaagtttcattgaacttacaccaccagccaagagagaaatcctatggttatccctgggaacgcagatcacctacatcggacttcaattcatcagcttcctcctgctactaacagacttgctactcactgggaaccctgcctgtgggctcaaactgagcgcctttgctgctgtttcctctgtcctgtcaggtctcctggggatggtggcccacatgatgtattcacaagtcttccaagcgactgtcaacttgggtccagaagactggagaccacatgtttggaattatggctgggccttctacatggcctggctctccttcacctgctgcatggcgtcggctgtcaccaccttcaacacgtacaccaggatggtgctggagttcaagtgcaagcatagtaagagcttcaaggaaaacccgaactgcctaccacatcaccatcagtgtttccctcggcggctgtcaagtgcagcccccaccgtgggtcctttgaccagctaccaccagtatcataatcagcccatccactctgtctctgagggagtcgacttctactccgagctgcggaacaagggatttcaaagaggggccagccaggagctgaaagaagcagttaggtcatctgtagaggaagagcagtgttaggagttaagcgggtttggggagtaggcttgagccctaccttacacgtctgctgattatcaacatgtgcttaagccaaaaaaaaaaaaaaaaaaaaaaaaaaaaaaaaaaaaaaaaaaaaaaaaaaaaaaaaaaaaaa

SEQ ID NO: 138 - protein, 339 aa, Homo sapiens
MSDPSQLTQNVCLTQEMELSKAFSGQRTLLSAILSMLSLSFSTTSLLSNYWFVGTQKVPKPLCEKGLAAKCFDMPVSLDGDTNTSTQEVVQYNWETGDDRFSFRSFRSGMWLSCEETVEEPGERCRSFIELTPPAKREILWLSLGTQITYIGLQFISFLLLLTDLLLTGNPACGLKLSAFAAVSSVLSGLLGMVAHMMYSQVFQATVNLGPEDWRPHVWNYGWAFYMAWLSFTCCMASAVTTFNTYTRMVLEFKCKHSKSFKENPNCLPHHHQCFPRRLSSAAPTVGPLTSYHQYHNQPIHSVSEGVDFYSELRNKGFQRGASQELKEAVRSSVEEEQC

SEQ ID NO: 139 - DNA, 3184 bp, Homo sapiens
  unsure (1644)
gtgcatgcttgtaatcgcagctacttcggagcctgagagactccttcagggtgagcaaaggcctggaaaaacctgtatgcagataaagaaaaggaaagaaagagataatcagtgcatgcagttgtcagctggctgggacctgaggagagtcacttgtggaggcaactggtctttatccccattgtccggtacaaggcaggcattaatcctgtgatccttatctgaagctcagctacaaggctttggccgaccaagtgtgtaccatgctgctattgtcatcttccttgaattctttgcgtggggcctgttgacaactccaatgttgactgttctacatgaaacattttctcaacacacattcctcatgaatggtctcattcaaggtgtaaagggcctgctctcttttttgagtgccccactcattggtgccctgtctgatgtgtgggggaggaagccctttctcctcggcactgtattctttacctgcttcccaatcccactgatgaggatcagcccatggtggtattttgcgatgatttctgtgtctggagtcttctcggtcacgttttctgttatatttgcctatgtagctgatgtcactcaggagcacgagcgaagtacagcttatggatgggtctcagccacctttgcggctagtcttgtcagcagcccggccattggagcatatctttctgccagttacggagacagcctcgttgtgctggtggccacagtggtggctcttctggacatctgcttcatcttagtggctgttccagaatctctgcctgagaaaatgagaccggtttcctggggagctcagatttcttggaaacaagcagacccttttgcgtcgttgaagaaagttggaaaagattctactgtcttactaatctgcatcaccgtgtttctttcataccttcctgaagctggacagtattcaagtttttttctctatctcaggcaggtcataggttttggatctgttaaaattgcagcattcatagctatggtaggaattctgtctattgtggctcagacggcctttcttagcatcttgatgagatcattaggaaataagaatactgtcctccttggcttgggcttccagatgctccagttagcctggtacggttttggatcacaggcctggatgatgtgggcagcagggaccgtggctgccatgtccagcatcacgtttccggcaatcagtgccctcgtctctcggaatgcagagtcagatcagcaaggagttgcccaggggatcataactggaataagaggactatgcaatggcctggggccagcactgtatggcttcatattctacatgttccatgtggaactgactgagttgggcccgaaattgaattctaacaacgttcccctgcagggagctgtcatcccaggcccgccgtttttatttggggcatgtatagtccttatgtcttttctggttgccttattcattcctgaatacagtaaagccagtggagttcaaaaacacagtaacagcagcagcggcagcctgaccaacaccccagaacggggcagtgatgaggacattgagccactactgcaagacagcagcatctgggagctctcttcatttgaggagcctgggaatcagtgcactgagctgtnaactcggcagaaagtgggattctgcatacgccatctctgagagccatggagggagccacacccctggtgacttcatggtgctggatgggagacgctagcggcatccttcagggccaagtttgataaataccaccgccatcattctgctcatcctcctcctgttttttttttttctcttacattctttttttttttcctgtttatacattagaacaagataagatttgaaatacttccttgcaaataatgtgcaactcccaaggtgaaactcaaatagaaaaagtcatctctctggtagaaaggatggctttcctgtaatgactatagagtaagagtggcagcaatctttccatgcccttttcagcagaaggcacagaacagtagcgggactgccatctctggcaagatttcaggtaaagaatctcttcttaatttctaccttcctgtttctctgaatcagcccataggtgttgatgagtggccactcttaaagagtcactcagtatcagggatctactgtctttgttcaaaggtcaaataaaaacctagtctccttttattctactttctattcttagctagaatgaaactcagcatatatacacttctggacataataatattgaatagtaattacctttactagatgaaagaaattttcattacaaacttaaatcatgtaaaactcaacaactcagattcctggacctggtgtcctggttgggtccaaggtgattttacagaagaaaaaaacaactcaagcattctggtggcaacatagagattgtaggctgcttctaagaaagttattaacaatttggaaattcctaagtaggatgagagttagtaactggatacgagtgaagtttatatccaagttcagactcaaaggcattattatgatttgcttcttcccatgtcttccatgtcctgcttctcaaagtttttctcatccatcacactcctgccttaactgctctgagtatgcatttgttttcaattcatctttatttcaatctgtttaacttttgaatcgcatgggaatacgcacattaagttcctttctaaaataaggttttatgaagctgagtttcacgataagtgtcttgctattttttgagatgttttatggacaaagaaaactttacagatttatatgtattttgctgcaccagtaaatggaccattaactagggcccacctttaacagagcacccctttgaaagttttataggtatgaaatatatgtagatatttgtaaagggttttaattttttttttttgatggggtgctgtgtaaatcttgtatttataaatgtaatgaaggtattgacagaaaaaaatatatacaacttttataaaggattgtgtactgactgaatacatttaaaagaaaatatattttgaaacctgttctgctatgaacagagataacatatctttttactatgctattggtttttaggttaagcttcctaatgcataataaatttacagtggttaaaaaaaaaaaaaaaaaaaaaaaaaaaaaaaaaaaaaa

SEQ ID NO: 140 - protein, 454 aa, Homo sapiens
  UNSURE (442)
MLTVLHETFSQHTFLMNGLIQGVKGLLSFLSAPLIGALSDVWGRKPFLLGTVFFTCFPIPLMRISPWWYFAMISVSGVFSVTFSVIFAYVADVTQEHERSTAYGWVSATFAASLVSSPAIGAYLSASYGDSLVVLVATVVALLDICFILVAVPESLPEKMRPVSWGAQISWKQADPFASLKKVGKDSTVLLICITVFLSYLPEAGQYSSFFLYLRQVIGFGSVKIAAFIAMVGILSIVAQTAFLSILMRSLGNKNTVLLGLGFQMLQLAWYGFGSQAWMMWAAGTVAAMSSITFPAISALVSRNAESDQQGVAQGIITGIRGLCNGLGPALYGFIFYMFHVELTELGPKLNSNNVPLQGAVIPGPPFLFGACIVLMSFLVALFIPEYSKASGVQKHSNSSSGSLTNTPERGSDEDIEPLLQDSSIWELSSFEEPGNQCTELXTRQKVGFCIRHL

SEQ ID NO: 141 - DNA, 2481 bp, Homo sapiens
aggtctagaattcaatcgggaagaaggaaaagttcccttctgctgtgaaactatttggcaagaggctggagggcccaatggctgcaaaatcgcaacccaacattcccaaagccaagagtctagatggcgtcaccaatgacagaaccgcatctcaagggcagtggggccgtgcctgggaggtggactggttttcactggcgagcgtcatcttcctactgctgttcgcccccttcatcgtctactacttcatcatggcttgtgaccaatacagctgcgccctgaccggccctgtggtggacatcgtcaccggacatgctcggctctcggacatctgggccaagactccacctataacgaggaaagccgcccagctctataccttgtgggtcaccttccaggtgcttctgtacacgtctctccctgacttctgccataagtttctacccggctacgtaggaggcatccaggagggggccgtgactcctgcaggggttgtgaacaagtatcagatcaacggcctgcaagcctggctcctcacgcacctgctctggtttgcaaacgctcatctcctgtcctggttctcgcccaccatcatcttcgacaactggatcccactgctgtggtgcgccaacatccttggctatgccgtctccaccttcgccatggtcaagggctacttcttccccaccagcgccagagactgcaaattcacaggcaatttcttttacaactacatgatgggcatcgagtttaaccctcggatcgggaagtggtttgacttcaagctgttcttcaatgggcgccccgggatcgtcgcctggaccctcatcaacctgtccttcgcagcgaagcagcgggagctccacagccatgtgaccaatgccatggtcctggtcaacgtcctgcaggccatctacgtgattgacttcttctggaacgaaacctggtacctgaagaccattgacatctgccatgaccacttcgggtggtacctgggctggggcgactgtgtctggctgccttatctttacacgctgcagggtctgtacttggtgtaccaccccgtgcagctgtccaccccgcacgccgtgggcgtcctgctgctgggcctggtgggctactacatcttccgggtggccaaccaccagaaggacctgttccgccgcacggatgggcgctgcctcatctggggcaggaagcccaaggtcatcgagtgctcctacacatccgccgacgggcagaggcaccacagcaagctgctggtgtcgggcttctggggcgtggcccgccacttcaactacgtcggcgacctgatgggcagcctggcctactgcctggcctgtggcggtggccacctgctgccctacttctacatcatctacatggccatcctgctgacccaccgctgcctccgggacgagcaccgctgcgccagcaagtacggccgggactgggagcgctacaccgccgcagtgccttaccgcctgctgcctggaatcttctaagggcacgccctagggagaagccctgtggggctgtcaagagcgtgttctgccaggtccatgggggctggcatcccagctccaactcgaggagcctcagtttcctcatctgtaaactggagagagcccagcacttggcaggtgtccagtacctaatcacgctctgttccttgcttttgccttcaagggaattccgagtgtccagcactgccgtattgccagcacagacggattttctctaatcagtgtccctggggcaggaggatgacccagtcacctttactagtcctttggagacaatttacctgtattaggagcccaggccacgctacactctgcccacactggtgagcaggaggtcttcccacgccctgtcattaggctgcatttactcttgctaaataaaagtgggagtggggcgtgcgcgttatccatgtattgcctttcagctctagatccccctcccctgcctgctctgcagtcgtgggtggggcccgtgcgccgtttctccttggtagcgtgcacggtgttgaactgggacactggggagaaaggggctttcatgtcgtttccttcctgctcctgctgmacagctgccaggagtgctctgcctggagtctgcagacctcagagaggtcccagcactggctgtggcctttcaggtgtaggcaggtgggctctgcttcccgattccctgtgagcgcccaccctctcgaaagaattttctgcttgccctgtgactgtgcagactctggctcgagcaacccggggaacttcaccctcaggggcctcccacaccttctccagcgaggaggtytcagtcccagcctcgggagggcacctccttttctgtgctttcttccctgaggcattcttcctcatccctagggtgttgtgtagaactctttttaaactctatgctccgagtagagttcatctttatattaaacttcccctgttcaaataaaaaaaaaaaaaa

SEQ ID NO: 142 - protein, 475 aa, Homo sapiens
MAAKSQPNIPKAKSLDGVTNDRTASQGQWGRAWEVDWFSLASVIFLLLFAPFIVYYFIMACDQYSCALTGPVVDIVTGHARLSDIWAKTPPITRKAAQLYTLWVTFQVLLYTSLPDFCHKFLPGYVGGIQEGAVTPAGVVNKYQINGLQAWLLTHLLWFANAHLLSWFSPTIIFDNWIPLLWCANILGYAVSTFAMVKGYFFPTSARDCKFTGNFFYNYMMGIEFNPRIGKWFDFKLFFNGRPGIVAWTLINLSFAAKQRELHSHVTNAMVLVNVLQAIYVIDFFWNETWYLKTIDICHDHFGWYLGWGDCVWLPYLYTLQGLYLVYHPVQLSTPHAVGVLLLGLVGYYIFRVANHQKDLFRRTDGRCLIWGRKPKVIECSYTSADGQRHHSKLLVSGFWGVARHFNYVGDLMGSLAYCLACGGGHLLPYFYIIYMAILLTHRCLRDEHRCASKYGRDWERYTAAVPYRLLPGIF

SEQ ID NO: 143 - DNA, 1518 bp, Homo sapiens
cttccccactggctcttggtttatgagttccccttttaaggatctgttgtgacttacctatctgggctagtgacctcagatgtctcagactgagcatcttaccactgtttctggttgatcccttcactcatggtcttaacacatttgcacttcctctcatctcagagagtacagtcacggggcagagcttgcatagggatccaggtgttactagtcttactctggagctggtccaactcagtttcatggcacagaactagattaggtctccactgcgcagtctgttttactgcttagggaaagccagcttttctacccacacacgtttagtttgaagagtatctatttttggagggttctttgggaggttgggcaggcttctttggatcccagatacatttagagctttttgcattaagtgtgaggaaaataacttctctttgatgatgttgatacaccatgtkggcaccytggggcacagcggtttagctggggagattccatgagaatgaacccaaactactcttctttgctagggtcctttacccacacagaggtgagcctttcaggttcttcattttgcttagtttcttcccttgtccttggcatttaagaggcatccatgtgttagccagccaaagccccctgaaggagctggctgctttaaaggatttacttgggaggatgtcaaatggctttgccttctgcagacttcatttattttaatctttttatggctcctttctcttgctttaaaacaggattataagcacacagcaggtactgacacctgaagtcttactaaattcctgtcctcaggccatcctttttctcctgaaacctggactccaattttcaatgacgtttttgtttttctctttcaagcctaactatgggacagctttacgagaaggaaaaagatgaagatggattcttatatgtggcctacagcggagagaacacttttggcttctgagggccattgctgggctaggtgcaccgtaactgcttgtgtatcttgtaaatagccasccattttcagttattawaccagaacctcttmacatagacctattagtgcatttgtaactggatttatttcttaatatatkggaaggttttgtttccttagactagtaaattatcatacagagttttattttgagtttttctttttgtgcattgtcctcatgcctgtattctccaggaaacttgtccttctggaaatcatatkgaatgatatttctatatcgaagtgaggtaggtgcggtattaaagtgaaagggaaggtgatgcatttattctgggttatgcttgaagtgttagatggctaagtattaaaattatccaaattaaatccttagcagtcagaacacttgcttcactagaatatgccaactgccaatcatgttggactgagctaatttgttcctctttctgaaactattaaggtaaataattaacaataaaaattctcttataaaggcaaaaaaaaaaaaaaaaaaaaaaaaaaaaaaa

SEQ ID NO: 144 - protein, 55 aa, Homo sapiens
MVLTHLHFLSSQRVQSRGRACIGIQVLLVLLWSWSNSVSWHRTRLGLHCAVCFTA

SEQ ID NO: 145 - DNA, 2097 bp, Homo sapiens
ctcttgagtacctggggcttgcagatgcatgccaccacacccggctaattttttttttttttaaatagagatggggtcttgttctgttgcccargctggtctggaactcctggcttcaatcagtcctcccacctcagcttcccaaagctctgggattataggcatgagccactgtacctgtccacctgagaaattttctaagcctggattcagtcttatgaaatataatactttgaaatgcacaataactttgaaaatgaaactcattgcttttcatttcaccaggagttactaactataataagctttagagcaaattctccttagatatgatttttgttattattagaaacacatactatcttgataactaaattttgccaatcattcttcttgactagtggtctttatatatacatacatatatatatatatatacatatatatatatatgaggaattttccataagtgacttgaaaaatacagaatgcactccatggtaggtctgttcagtgttatcaggaatactgtttctcatcttcctttcttggtgtccctttgcaggggttgtgtttgcacattatggtcccgtctggagacaacaaaggaagttctctcattcaactcttcgtcattttgggttgggaaaacttagcttggagcccaagattattgaggagttcaaatatgtgaaagcagaaatgcaaaagcacggagaagaccccttctgccctttctccatcatcagcaatgccgtctctaacatcatttgctccttgtgctttggccagcgctttgattacactaatagtgagttcaagaaaatgcttggttttatgtcacgaggcctagaaatctgtctgaacagtcaagtcctcctggtcaacatatgcccttggctttattaccttccctttggaccatttaaggaattaagacaaattgaaaaggatataaccagtttccttaaaaaaatcatcaaagaccatcaagagtctctggatagagagaaccctcaggacttcatagacatgtaccttctccacatggaagaggagaggaaaaataatagtaacagcagttttgatgaagagtacttattttatatcattggggatctctttattgctgggactgataccacaactaactctttgctctggtgcctgctgtatatgtcgctgaaccccgatgtacaagaaaaggttcatgaagaaattgaaagagtcattggcgccaaccgagctccttccctcacagacaaggcccagatgccctacacagaagccaccatcatggaagtgcagaggctaactgtggtggtgccgcttgccattcctcatatgacctcagagaacacagtgctccaagggtataccattcctaaaggcacattgatcttacccaacctgtggtcagtacatagagacccagccatttgggagaaaccggaggatttctaccctaatcgatttctggatgaccaaggacaactaattaaaaaagaaacctttattccttttgggatagggaagcgggtgtgtatgggagaacaactggcaaagatggaattattcctaatgtttgtgagcctaatgcagagtttcgcatttgctttacctgaggattctaagaagcccctcctgastggaagatttggtctaactttagccccacatccatttaatataactatttcaaggagatgaagagcatctccaagaagagatggtaaaaagatatataaatacatatccttctaagcagattcttcctactgcaaaggacagtgaatccagcaactcagtggatccaagctgggctcagaggtcggaaggagggtagagcacactgggaggtttcatcttggaggattcctcagcaggatacttcagccattttagtaatgcaggtctgtgatttgggggatagaaaacaaagtacctatgaaacgggatatctggattttacttgcagtggcttccaccgatgggccaatcttctcatttcttagtgcctcagacatcccatatgtaaaatgagagtaataaaacttggcttctctctacctctcagcactaaaaaaaaaaaaaaaa

SEQ ID NO: 146 - protein, 398 aa, Homo sapiens
  UNSURE (379)
VLSGILFLIFLSWCPFAGVVFAHYGPVWRQQRKFSHSTLRHFGLGKLSLEPKIIEEFKYVKAEMQKHGEDPFCPFSIISNAVSNIICSLCFGQRFDYTNSEFKKMLGFMSRGLEICLNSQVLLVNICPWLYYLPFGPFKELRQIEKDITSFLKKIIKDHQESLDRENPQDFIDMYLLHMEEERKNNSNSSFDEEYLFYIIGDLFIAGTDTTTNSLLWCLLYMSLNPDVQEKVHEEIERVIGANRAPSLTDKAQMPYTEATIMEVQRLTVVVPLAIPHMTSENTVLQGYTIPKGTLILPNLWSVHRDPAIWEKPEDFYPNRFLDDQGQLIKKETFIPFGIGKRVCMGEQLAKMELFLMFVSLMQSFAFALPEDSKKPLLXGRFGLTLAPHPFNITISRR

SEQ ID NO: 147 - DNA, 2504 bp, Homo sapiens
gtcactgtgagtggagcccatgctgggctctgtgccctctgtgtctgtgcatgcgcgtgtgtgtgtgggcgtgtgtgcattgctgggccagcttgaagggaaggcccgtcatgtccctgcactctgttttgcaagatgccaaaccccagttctgatggggctccaacagccaggctgtggtcctttgacgttcctcacctgttgccaacctatcccgtagtgaactgaaaccccaatgaagacagaactgtgcctggggagatgcaatgaggtgagggctgaactcatccttttatatttcttttcaagattggatcagagctcatctccatccagtcttgtttctatgaaggcttcaatctgtttccatgcaaatttgctaatcagagcccagagctgctgggtccctcatctccctcatctattatagattgacttacagcagggagagaatctctttagctcattcctaatggggttgggatcacaatatggtctggtccaatctgcatcttgttgtgtcccaagaccctatctcctccccaacattcttattgcctttggctcccagtaaggaacgaattgggggccagggaggagaacaggggggatcaagaagggaaacccaattccccctttgaaagtgggttctttgaactatgtgtttgggggaagttcctctggatactaatttgaatttatatacctcatgttttgggggtttgacctatatatatatatatatatatatatgcatatatatttcataatatttggaaggtttttgatgctagaaaaatggaaacaagagaaccttcaaaaatggtacttagatgggaactggaggccaatctttcataaagccagccccatagctgcttgctgttaggcctccagccattttgacattggggtggatagtcgattcacctgcctgtcagtcgattcacctgcctgtcacccagttctgtggatgtgctggtgctgagcctttgctctctttccaaatggttacagggatgttgatcagctccaccagagggagctctgatgggaggaattgctctgccatccttgtccctgtgtctcctgtcggcaggcagccattgtatctcaccagcagaccaggagactggtcccaaggttactgcaccacagggcaatttcctgccatagttaggaaggaaacacctgaactaaatggaagagacatccctgcggtgtttaatatcacacccatgccctttgtcaggttaccatgtacagagattacttggagagcctcatgccgtctctaccttcgcacactggtcaagtatctgctgagcttcttggccgcaaggatgcagaaataggctgagggtccatgggaagaaagacacaatgaggcagtaggaggtgggaagaaaagaagacagactttcaaaatggaattaggcactggggagagatcagtttccccacatcagggagaagaaggtataggtggggaagggggtggccaggagcagaaggaagaagactcaagatggaaagggagccgctgtgcctgtggcaataccacttggagaggtcgacttcataccttcaagccttttcccctgggcttttgattgtgtctgtgccccctttcttgtcctctctgcagatgcccagtaggggctacctcatcctcgtgctgttcttgtgtggctttctgggcagtagggatcttgaatttcctttctaacactgtgcccggcaaggcggggagcattcctctgccctttgtcttgtgccaacctggaaaggtgcagtctagatttcagtgagaaccctgccagctgagccctgtgcatctactaccttgacacagagtgttttcccactagaagctctgctctgctctcctggcccaagtaggggattccatgccttccctttcatggtcttagcaccagcagcctagtttctcccttccagagtctccagggatgacaaattggattggagacaaacctcgtcagatgctcatcccctaaaaggttaattgtgtatttgtggctgcgtgtgcctttgtgttttcattctcttcccatttttgtacattttggtcttctctgtggttttatacttggtcaaaagtactcgtcttggtattgcactgttgtgtgcatgagaaaactgggggaaggctcactggtacaagaaaggacccctgacccctttccttctctgtggtccccggcattagattgggggttctgggagaggcaggtgaatgtcctaagtgaattgttctgtttgtaactggaatgtttttgaagtctttggtgttgctccgtgaaaggacatcgccacctggtgctcatgaggtgtctttgcagaacaataaatggcaaatgaacaaccccccccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 148 - protein, 66 aa, Homo sapiens [sequence omitted]

SEQ ID NO: 149 - DNA, 928 bp, Homo sapiens
caagaccagtcttgccaacataacaagaatctgtctctatataagaagattaagaattggctgggcatggtggcatgtgcttgtggccctagctacttgggaggctgcggtggaaggatcacttgggcccaggcattccagcttatgatttcagtgagttatgatcacaacactgaattccaacctaatggatggagagagactatgtctctaaaaataaaaaataaagagattaggaactgtctgcactaagatgactttactattccaagaaatccttgcctaagaaagtaaagttgaaattacttttttgtcctggaaactttccgatctatgtatctgtactcatacagcctcatcgggctaaacagccttcttttcagaacagtagatcactcaactgggttttcaagtgactgtttacctttcaaggctggctttataggtcttgcctcactgtatccagcaatccaaactttaccctatcccagtcaggactgcacacctcatgttgaaagacataccttagaaccagactccccaaagcttacaaatatcccacccttgactcccttttctgaggctactaagattatgtgaagacagtcatcttccttactgcagtgagcaataaacttggtttttgttcatcagtaaaccattttggtggtttctggaggagccagcagttggcaatggttataaatctaaatctaaaagccatttataaaagactgatgaatctagtaacataaaaataaactgcatgataaatatcataaacaaagtcaaaagacaactgacaaccaggttaaaaacatgctttcaacatatattacaggaaaagggctaatattcctaatatgtaaataattgttagaaattaagagatcaagcaccaagcacccattagaaaaaaaaaaaaaaa

SEQ ID NO: 150 - protein, 88 aa, Homo sapiens [sequence omitted]

SEQ ID NO: 151 - DNA, 1343 bp, Homo sapiens
ccgagccagggttccctgccggccttggagatggcgggacttcccacgtctggagccgaggcctggataattcggatttggcacgggaaacatcttggtcgtttgccatttttcggctttggggagtgtttgcgtttcttctccgtttggcagtgaaacacatctcagaaaggtggagctgatcagaataatgttcagcatcaaccccctggagaacctgaaggtgtacatcagcagtcggcctcccctggtggtcttcatgatcagcgtaagcgccatggccatagctttcctgaccctgggctacttcttcaaaatcaaggagattaaatccccagaaatggcagaggattggaatacttttctgctacggttcaatgatttggacttgtgtgtatcagagaatgaaaccctcaagcatctcacaaacgacaccacaactccggaaagtacaatgaccagcgggcaggcccgagcttccacccagtccccccaggccctggaggactcgggcccggtgaatatctcagtctcaatcaccctaaccctggacccactgaaacccttcggagggtattcccgcaacgtcacccatctgtactcaaccatcttagggcatcagattggactttcaggcagggaagcccacgaggagataaacatcaccttcaccctgcctacagcgtggagctcagatgactgcgccctccacggtcactgtgagcaggtggtattcacagcctgcatgaccctcacggccagccctggggtgttccccgtcactgtacagccaccgcactgtgttcctgacacgtacagcaacgccacgctctggtacaagatcttcacaactgccagagatgccaacacaaaatacgcccaagattacaatcctttctggtgttataagggggccattggaaaagtctatcatgctttaaatcccaagcttacagtgattgttccagatgatgaccgttcattaataaatttgcatctcatgcacaccagttacttcctctttgtgatggtgataacaatgttttgctatgctgttatcaagggcagacctagcaaattgcgtcagagcaatcctgaattttgtcccgagaaggtggctttggctgaagcctaattccacagctccttgttttttgagagagactgagagaaccataatccttgcctgctgaacccagcctgggcctggatgctctgtgaatacattatcttgcgatgttgggttattccagccaaagacatttcaagtgcctgtaactgatttgtacatatttataaaaatctattcggaaaaaaaaaaaaaaaaaaaaaaaaaaaaaaaa

SEQ ID NO: 152 - protein, 314 aa, Homo sapiens
MFSINPLENLKVYISSRPPLVVFMISVSAMAIAFLTLGYFFKIKEIKSPEMAEDWNTFLLRFNDLDLCVSENETLKHLTNDTTTPESTMTSGQARASTQSPQALEDSGPVNISVSITLTLDPLKPFGGYSRNVTHLYSTILGHQIGLSGREAHEEINITFTLPTAWSSDDCALHGHCEQVVFTACMTLTASPGVFPVTVQPPHCVPDTYSNATLWYKIFTTARDANTKYAQDYNPFWCYKGAIGKVYHALNPKLTVIVPDDDRSLINLHLMHTSYFLFVMVITMFCYAVIKGRPSKLRQSNPEFCPEKVALAEA

SEQ ID NO: 153 - DNA, 3343 bp, Homo sapiens
tcccgcgccggggccgcgggcggagctgcctgccggtcccgcgccgcgcgtccgcactcctcggccctcgggcggtcgatgggacggggcgccgcggagcaggaggcggcgcccgtcggggtgctcgggccgcgcgggagcccactgtggggctcgggcatggcgggccgcaggacctgagctctcctcaggggagcggggaggcagctgctggccggcgatggggacggagtggggccgtcgccgccgcgccgagccgtgagcgccgagccaccgccgccgctacctcagcccttcgcgaagcgccgggcagctcgggaacatggccctggagcggctctgctcggtcctcaaagtgttgttaataacagtactggtagtggaagggattgccgtggcccaaaaaaacccaagatggacaaaatattggaatcaagcatattcctgcaacccagtgtggcatttgggttcgaaccagcaatggaggtcattttgcttcgccaaattatcctgactcatatccaccaaacaaggagtgtatctacattttggaagctgctccacgtcaaagaatagagttgacctttgatgaacattattatatagaaccatcatttgagtgtcggtttgatcacttggaagttcgagatgggccatttggtttctctcctcttatagatcgttactgtggcgtgaaaagccctccattaattagatcaacagggagattcatgtggattaagtttagttctgatgaagagcttgaaggactgggatttcgagcaaaatattcatttattccagatccagactttacttacctaggaggtattttaaatcccattccagattgtcagttcgagctctcgggagctgatggaatagtgcgctctagtcaggtagaacaagaggagaaaacaaaaccaggccaagccgttgattgcatctggaccattaaagccactccaaaagctaagatttatttgaggttcctagattatcaaatggagcactcaaatgaatgcaagagaaacttcgttgcagtctatgatggaagcagttctattgaaaacctgaaggccaagttttgcagcactgtggccaatgatgtaatgcttaaaacaggaattggagtgattcgaatgtgggcagatgaaggtagtcggcttagcaggtttcgaatgctctttacttcctttgtggagcctccctgcacaagcagcactttcttttgccatagcaacatgtgcatcaataattctttagtctgtaatggtgtccaaaattgtgcatacccttgggatgaaaatcattgtaaagaaaagaaaaaagcaggagtatttgaacaaatcactaagactcatggaacaattattggcattacttcagggattgtcttggtccttctcattatttctattttagtacaagtgaaacagcctcgaaaaaaggtcatggcttgcaaaaccgcttttaataaaaccgggttccaagaagtgtttgatcctcctcattatgaactgttttcactaagggacaaagagatttctgcagacctggcagacttgtcggaagaattggacaactaccagaagatgcggcgctcctccaccgcctcccgctgcatccacgaccaccactgtgggtcgcaggcctccagcgtcaaacaaagcaggaccaacctcagttccatggaacttcctttccgaaatgactttgcacaaccacagccaatgaaaacatttaatagcaccttcaagaaaagtagttacactttcaaacagggacatgagtgccctgagcaggccctggaagaccgagtaatggaggagattccctgtgaaatttatgtcagggggcgagaagattctgcacaagcatccatatccattgacttctaatcttctgctaatggtgatgtgaattcttagggtgtgtacgtacgcagcctccagggcaccatactgtttccagcagccaacccttttctcccatcacaactacgaagaccttgatttaccgttaacctattgtatggtgatgtttttattctctcaggcagtctatatatgttaaaccaatcaaggaacttactctattcagtggaaacaataatcatctctattgcttggtgtcatttataggaagcactgccagttaaagagcattagaagaggtggttggatggagccaggctcaggctgcctcttcgttttagcaacaagaagactgctcttgactgataacagctctgtcaatattttgatgccacaataaacttgatttttctttacattccttttatttttcctttctctaaatttaatttgttttataagcctatcgttttaccatttcattttcttacataagtacaagtggttaatgtaccacatacttcagtataggcatttgttcttgagtgtgtcaaaatacagctagttactgtgccaattaagacccagttgtatttcacccatctgtttcttcttggctaatctctgtacttctgccttttaattactgggcccttattccttattttctgtgagaaataatagatgatatgatttattacctttcaattatatttttctcagttatactagaaaatttcataatcctgggatatatgtaccattgtcagctatgactaaaaatttgaaaaagataaaaatttctagcaagcctttgaagtttaccaagtatagtcacattcagtgacagcccattcattccagtaaagaatcatttcattcactttgggagaggcctataatwacatttatttgcaatgtttctcttcgctagattgtwacatagctcccattctgttggttttgcttacagcatatggtaaccaaggttagatgccagttaaaattccttagaaattggatgagccttgagattgcttcttaactgggacatgacatttttctagctcttatcaagaataacaacttccacttttttttaaactgcacttttgactttttttatggtataaaaacaataatttataaacataaaagctcattgtgttttttagacttttgatattatttgatactgtacaaactttattaaatcaagatgaaagacctacaggacagattcctttcagtgttcacatcagtggctttgtatgcaaatatgctgtgttggacctggacgctataacttattgtaaagaccttggaaatgtggacataagctctttctttccttttgttactgtatttagtttgtgataaatttttcactgtgtgatatttatgctctaaatcactacacaaatcccatattaaaatatacattgtacctgaaaaaaaaaaaaaaa

SEQ ID NO: 154 - protein, 389 aa, Homo sapiens
MWIKFSSDEELEGLGFRAKYSFIPDPDFTYLGGILNPIPDCQFELSGADGIVRSSQVEQEEKTKPGQAVDCIWTIKATPKAKIYLRFLDYQMEHSNECKRNFVAVYDGSSSIENLKAKFCSTVANDVMLKTGIGVIRMWADEGSRLSRFRMLFTSFVEPPCTSSTFFCHSNMCINNSLVCNGVQNCAYPWDENHCKEKKKAGVFEQITKTHGTIIGITSGIVLVLLIISILVQVKQPRKKVMACKTAFNKTGFQEVFDPPHYELFSLRDKEISADLADLSEELDNYQKMRRSSTASRCIHDHHCGSQASSVKQSRTNLSSMELPFRNDFAQPQPMKTFNSTFKKSSYTFKQGHECPEQALEDRVMEEIPCEIYVRGREDSAQASISIDF

SEQ ID NO: 155 - DNA, 2991 bp, Homo sapiens
  unsure (1270)
  unsure (2613)
ggcatggctattgcaccttgggagaagcctttaatcggttagacttctcaagtgcaattcaagatatccgaaggttcaattatgtggtcaaactgttgcagctaattgcaaaatcccagttaacttcattgagtggcgtggcacagaagaattacttcaacattttggataaaatcgttcaaaaggttctttgattaagcgaggattgtggtggtcatcaagaaccttttcccgattgaattctagacctgcggggtagttgcctttggccaaaccaaggacatcatcaggcagatcctgcaggctgatggacttcgcggcttctatcgaggctatgtggcttcactgcttacctatatcccaaacagtgctgtctggtggcccttctatcacttctatgcaggttgagggcaagaactccatcatcctgaccttcagacagctgatggcagaagaagggccttggggcctcatgaaaggcctctcggccagaatcatctcagccacaccttccaccattgtcattgtggtgggctatgagagcctcaagaaactcagcctccgacctgagctggtggactcgagacactggtaaccagtggtggggagagaagcctgctgttttccacactaccgtgggtcaggggcagagtggagaggacagcaccctctccaggtgctcccaccacacacccagccctgccctgggccaagtggcctatctgggatagggatagagactttgaactgctcttgctgaagaggctccacgcctggatcccttgcccccactatttaaaattctcttctgagctgggctccctcactcagtccctgtatttgatactggcctaaagaccccaccccccaccctgccagcccttcttctggcttccccttccatctgtgtccctgagaccctgagaagagctgtacatagagcttgcttactaccactggttcttcctcttgggctttcagcccagactccaagcagctgctatcaaccctctctcccttcatctcttagccttgcttatttttattttgggaccgagctgcccactagatgactctgcttttccctgcatttggggctaaggtgccaggtacttatttgcacagggagcaggagcagcaaaaaatctctggttctccagagcactcgtcctctctttggaggggttattaggttgggagaaatgttgatacttttgttttgtgtgtgtgtgtgtgtgtgtgtgtgtgtgtgtgtgtgtgtgtgtgtttnaacatctgtgaaccaggctattagtcctgctaaagcgccaatcctgctgtcagagctcacccccttcctaagacaggtagaaaaatgtaatgtagctttttccacaagccacttccctgtcccttcagtctcaggagccctaggagagtctaagctggggcatcccctggcccagaggactcccgtggtgggcacagttctaagtggatcaggctgtcttgggtgcactggacttggagcactaccttgagaagtcaggttgagaaagtagttgatctagaaggcaacaagtgggcatgtgttccccagcacattacccaggccagcagagccaaacctaggagagggcagtgggtagattctctgccccaggcagccatgacatacacataaataccccaatcactcagacttacggcaacaagtgttgtctcactatggtgatctctaagatccacatcactggatgcgtagtcatcccagtcatggtaccctgtggaggaatgctggaagaacataaagagcagttcagaaagtcacccaataccaggaccactgcatttaccagcctgatactgccaagattatctgatgctctcctcaggagctaggagaggagtgctccttcctccctaccgctactctccccaagcctgtgttgcaggtagagaggtgcagcaaatagagaaggcatgtcaaaccctgcatttctacctgagacgtgtgacctggatgatcctccaaaccctattggtcccaccccctgggaaaggccatggtgccagtttgaaaggtgctagctacctgaagccttgatatttcttcatgggtgccgcacattcttccaccttggccagaacaggttctgaaaaccacttctctaccttcaccaccaccactgcccatcttgatctctttgagggttttcccatttcacttgatcttattttggtttatcccttcctgcactttgtcaagagagtcctccagtttctatccaggaatgttcacatccaaagggttggacccacggatcattctgaatcttcctgcccctcctcagtgcttaaccctgagaaccacaaatataatggaagcagttccccccaccctcaccccatctctttaagctcatcctagcaagacctctagagaccctagagactcgactttagtccttccccgccatggcacagtggggaaggtgtcaatggggagtgtcacggacaggaggtaggatcctgccgctcgcgtcttagtgtttctccctcaagactttccttctgttttgttgtcttgtgtagtattttacagcccctcttgtgtttttctttatttctcgnacacacacgcagttttaagggtgatgtgtgtataattaaaaggacccttggcccatactttcctaattctttagggactgggattgggtttgactgaaatatgttttggtggggatgggacggtggacttccattctccctaaactggagttttggtcggtaatcaaaactaaaagaaacctctgggagactggaaacctgattggagcactgaggaacaagggaatgaaaaggcagactctctgaacgtttgatgaaatggactcttgtgaaaattaacagtgaatattcactgttgcactgtacgaagtctctgaaatgtaattaaaagtttttattgagcccccgaaaaaaaaaaaaaaaaaaaaaaaaaaaaaa

SEQ ID NO: 156 - protein, 95 aa, Homo sapiens
MDFAASIEAMWLHCLPISQTVLSGGPSITSMQVEGKNSIILTFRQLMAEEGPWGLMKGLSARIISATPSTIVIVVGYESLKKLSLRPELVDSRHW

SEQ ID NO: 157 - DNA, 2293 bp, Homo sapiens
cacctgctgcccaccaccccggcagcacctttccctgcccaggcttcagagtgccctgttgctgctgccactgccccccacactccagggccatgtcagagctcccatctaccctccaccagcatgccgctcctgaagatgcccccaccattctcggggtgcagccacccctgcagcgggcactgtggtgggcactgcagtgggcctctcctcccacccccgagctctcagccactccctagcactcacagggatcccgggtgcaaggggcacaagtttgcacacagtggcctggcttgccagctgccccagccctgcgaggcagatgaggggctgggtgaggaagaggatagcagctctgagcgaagctcctgcacctcatcctccacccaccagagagatgggaagttctgtgactgctgctactgtgagttcttcggccacaatgcgccacccgctgccccgacgagtcggaactataccgagatccgggagaagctccgctcgaggctgaccaggcggaaagaggagctgcccatgaaggggggcaccctgggcgggatccctggggagcccgccgtggaccaccgagatgtggatgagctgctggaattcatcaacagcacggagcccaaagtccccaacagcgccagggccgccaagcgggcccggcacaagctgaaaaagaaggaaaaggagaaggcccagttggcagcagaagctctaaagcaggcaaatcgtgtttctggaagccgggagccaaggcctgccagggagaggctcttggagtggcccgaccgggaactggatcgggtcaacagcttcctgagcagccgtctgcaggagatcaaaaacactgtcaaagactccatccgtgccagcttcagtgtgtgtgagctcagcatggacagcaatggcttctctaaggagggggctgctgagcctgagcctcagagtctacccccctcaaacctcagtkgctcctcagagcagcagcctgacatcaaccttgacctgtcccctttgactttgggctcccctcagaaccacacgttacaagctccaggcgagccagccccaccatgggcagaaatgagaggcccccacccaccatggacagaggtgagggggccccctcccggtatcgtccccgagaacgggctcgtgaggagactcaacaccgtgcccaacctatcccgggtgatctgggtcaagacacccaagccgggctaccccagctccgaggagccaagctcaaaggaagttcccagttgcaagcaggagctgcctgagcctgtgtcctcaggtgggaagccacagaagggcaagaggcagggcagtcaggccaagaagagcgaggcaagcccagccccccggcccccagccagcctagaggttcccagtgccaagggccaggtcgctggccccaagcagccaggcagggtcctagagcttcccaaagtaggcagctgtgctgaggctggagaggggagccgggggagccggccaggaccaggttgggctggcagtcccaaaactgagaaggagaagggcagctcctggcgaaactggccaggcgaggccaaggcacggcctcaggagcaggagtctgtgcagcccccaggcccagcaaggccacagagcttgccccagggcaagggccgcagccgccggagccgcaacaagcaggagaagccagcctcctccttggacgatgtgttcctgcccaaggacatggacggggtggagatggatgagactgaccgagaggtggagtactttaagaggttctgtttggattctgcaaagcagactcgtcagaaagttgctgtgaactggaccaacttcagcctcaagaaaaccactcctagcacagctcagtgaggccctgcccaggctgagctgcttcagggcatcctgaggccctgactgccagctgaaggcgtataatttttccctccgtgtgccccacmtacccgtccaagaccctctgtgctccccaccatcctggaccaaccaaaagctgaacggatgccacactgtgctggggccccttgacctcagcagagccgcttcctggtgctacgcagcctccacactcagagcccgtggactgggctggcctaagggccagggctgatggtactgctggcccaacactgctctctttgtgtttggtttttttgtttttgtttttattttgtttttttccaattctttacttttgatactgtgaagatctttcgtgccgaaagataaagcaacatttggacacagaaaaaaaaaaaaaaaa

SEQ ID NO: 158 - protein, 586 aa, Homo sapiens
  UNSURE (286)
MPLLKMPPPFSGCSHPCSGHCGGHCSGPLLPPPSSQPLPSTHRDPGCKGHKFAHSGLACQLPQPCEADEGLGEEEDSSSERSSCTSSSTHQRDGKFCDCCYCEFFGHNAPPAAPTSRNYTEIREKLRSRLTRRKEELPMKGGTLGGIPGEPAVDHRDVDELLEFINSTEPKVPNSARAAKRARHKLKKKEKEKAQLAAEALKQANRVSGSREPRPARERLLEWPDRELDRVNSFLSSRLQEIKNTVKDSIRASFSVCELSMDSNGFSKEGAAEPEPQSLPPSNLSXSSEQQPDINLDLSPLTLGSPQNHTLQAPGEPAPPWAEMRGPHPPWTEVRGPPPGIVPENGLVRRLNTVPNLSRVIWVKTPKPGYPSSEEPSSKEVPSCKQELPEPVSSGGKPQKGKRQGSQAKKSEASPAPRPPASLEVPSAKGQVAGPKQPGRVLELPKVGSCAEAGEGSRGSRPGPGWAGSPKTEKEKGSSWRNWPGEAKARPQEQESVQPPGPARPQSLPQGKGRSRRSRNKQEKPASSLDDVFLPKDMDGVEMDETDREVEYFKRFCLDSAKQTRQKVAVNWTNFSLKKTTPSTAQ

SEQ ID NO: 159 - DNA, 1704 bp, Homo sapiens
ccggagggcagaaggcagartccaggcttagactgcagttcctcgcttacctgtgcagtctaattttgagctgcctctttgtagtcttaaaaggcaggagcttcgtgttgtgggtctgctaacccgtacgtttccgtgggcaagtcgtgtgtactcctcgccatggctcagctccaaacacgcttctacactgataacaagaaatatgccgtagatgatgttcccttctcaatccctgctgcctctgaaattgccgaccttagtaacatcatcaataaactactaaaggacaaaaatgagttccacaaacatgtggagtttgatttccttattaagggccagtttctgcgaatgcccttggacaaacacatggaaatggagaacatctcatcagaagaagttgtggaaatagaatacgtggagaagtatactgcaccccagccagagcaatgcatgttccatgatgactggatcagttcaattaaaggggcagaggaatggatcttgactggttcttatgataagacttctcggatctggtccttggaaggaaagtcaataatgacaattgtgggacatacggatgttgtaaaagatgtggcctgggtgaaaaaagatagtttgtcctgcttattattgagtgcttctatggatcagactattctcttatgggagtggaatgtagagagaaacaaagtgaaagccctacactgctgtagaggtcatgctggaagtgtagattctatagctgttgatggctcaggaactaaattttgcagtggctcctgggataagatgctaaagatctggtctacagtccctacagatgaagaagatgaaatggaggagtccacaaatcgaccaagaaagaaacagaagacagaacagttgggactaacaaggactcccatagtgaccctctctggccacatggaggcagtttcctcagttctgtggtcagatgctgaagaaatctgcagtgcatcttgggaccatacaattagagtgtgggatgttgagtctggcagtcttaagtcaactttgacaggaaataaagtgtttaattgtatttcctattctccactttgtaaacgtttagcatctggaagcacagataggcatatcagactgtgggatccccgaactaaagatggttctttggtgtcgctgtccctaacgtcacatactggttgggtgacatcagtaaaatggtctcctacccatgaacagcagctgatttcaggatctttagataacattgttaagctgtgggatacaagaagttgtaaggctcctctctatgatctggctgctcatgaagacaaagttctgagtgtagactggacagacacagggctacttctgagtggaggagcagacaataaattgtattcctacagatattcacctaccacttcccatgttggggcatgaaagtgaacaataatttgactatagagattatttctgtaaatgaaattggtagagaaccatgaaattacatagatgcagatgcagaaagcagccttttgaagtttatataatgttttcacccttcataacagctaacgtatcactttttcttatttkgtatttataataagataggtkgtgtttataaaatacaaactgtggcatacattctctatacaaacttgaaattaaactgagttttacatttcttctttaaaaaaaaaaaaaaaa

SEQ ID NO: 160 - protein, 423 aa, Homo sapiens [sequence omitted]

SEQ ID NO: 161 - DNA, 2302 bp, Homo sapiens
cgttggcaagcaattcacaaggtggggacagacttgtactttaacatgtagtccattcaagcaaacaactttggactctactgatagatgaaagagcaaatgatgactagtttagcctctgcatatcaacaatataatgcagatcaagtataatgctcaatattagtgacatgagtatcactaaattacatagaaccctgatggggtttcctgtgtcgtaatccattaaatcggtggccagtgcttgctgccgtggtttagtgattgggtgttagaaataaaaactcaggtctatttcttaccagtcagtaacaatttttagagaatgtacttggtatataatatatggacttcaggaactttattggggtggggggttaattttgccttaccctgttcactttcagatgawtaggcttttgcactttagaatgagaaacttgtgacgttagtgtgttcttactagctttaatttgtatgttagcaatgaattgtgaatcttagtgcagtgggtttttttaaaaaactcaaaaagctgggaattaagtggtttcagtaataatgctataccgaggtgcttgcattgtatttcataattttgttacaaaccaaaattatttttaatgagaacagtcttgggttcagaggtgtgatgccagaatgtattttcgtactgttaggcccttggaacagataccggtgctttctgaaagatgaaagaaatgcaatgggtgctcttcatgcaaggttgcaaacctaccaagaatgcataatagtctcacttttccccaataaagagatgcgtgtgactagttttggacttttaaccttaatgggggttgcatgtctcctattgttaatcattgtcagctgcagtgacatgatccacagtcctgcatttactgcctttcacttaatgattttggacaggttttagagagccaggatgttggtctgggcctttatttggttttggctttagctgataatgttttagtgatgtcagctagtgcagttctcaaatggctgcctattagggaaagaattcagaggatttgactgctcctaatcatctgtcattgctgctagataatgattggcaatttttaagactcaactggaaatctcaacagttgctggtaaaccattaaccataaaaacgttgcttttgaacaccagtgctgaaaaaaatatttttttttttttttttgagagtgaaaagggcttggacttaagataggacaatgtggagaatggggggaagaatgcaaaacgatatagtatcccttatggatggtacatgtgcaacagggaactcttacttcatataccytttgcagtaatcattcagggaggaagaaaaacstggaacttgaatgaaggctgatctttgttttgtgcactgtggccctgccaggcatatagtgaaggtgaatgtcttctccctcagaaaaaaattggttccttgctgtcccagtaaggcatagcttttccagccctaactttaaaactcagtgaggacttagatgggaaagaatgaggtaaatacaaaggattgcaggacaacaactacagcgttgtgtactgtgggaaggggagttgggcactcttgggaggactcctgctgaaggtggtcagcctgcctgacaatggaagacatacttgaatggggagcagggtatgtgctttcatatgaaaaaagagctgatgttaaaactcatttggtgaggtcaacgttgtcacataccttcacataagggatagtwtattttgggttgcagtcaaacttgtgctcagactggtgaaactgagagtcaggcttttacattttaaagaaaatacagtattcattctaattcaggtgtctacttattttatgtaagaataattttagatttcccccccaccatgaagtttcttcctattttcttatgctgtaacttacccccaatctttatctctggatttttactctttaaattttgaagttgactagcattttcaaacctttattttatacccttgtcttttatatwaactttttcttattattctttaggtaagaatgattgatgttggctgatattggagtgctcattcacatgaagtggatagatacttctcaagacatcacacagcgtgagtcaatcaaggagggaagccacaagcagactgacaacgtttctagcaggatcaggtgagctgtgtccagaaaaccaacgagaaggagtggaaggaggaatgaacgtttcattctcgttaataaaggcattatcctaattaaaaaaaaaaaaaaaaaaaa

SEQ ID NO: 162 - protein, 94 aa, Homo sapiens
MPECIFVLLGPWNRYRCFLKDERNAMGALHARLQTYQECIIVSLFPNKEMRVTSFGLLTLMGVACLLLLIIVSCSDMIHSPAFTAFHLMILDRF

SEQ ID NO: 163 - DNA, 1538 bp, Homo sapiens
rcagcctgctgcgcgcccaggggtcccgcgggttttcgggcgcagggtggcscccgcggcaggcggcggccatgaacttctccgaggtattcaagctctccagcttactctgcaagttctccccggacrgcaagtacctggcttcctgtgtccagtaccggttagtggtccgggatgtgaacacccttcagatccttcagctgtacacgtgcctagaccagatccagcacatcgagtggtcggcagactcgctcttcatcctgtkcgccatgtacaarcgagggctggtgcaggtctggtctttagagcagcccgaatggcactgcaaaatagacgagggctcagccgggctggtggcctcgtgctggagcccggacgggcgccacattctcaacaccacggaattccatctgcggataaccgtctggtccttgtgcacaaaatccgtgtcttacatcaaatacccgaaagcttgtctgcagggaatcaccttcaccagggacggccgctacatggcgctggcagaacggcgcgactgcaaagattacgtgagcatcttcgtctgcagtgattggcagctcctgcggcattttgatacggacacccaggatctcacagggattgagtgggccccaaacggctgtgtgctggcagtgtgggacacctgcttggaggtgcgcatccttaatcacgtgacttggaaaatgatcacggagtttgggcatcctgcagccattaatgatcccaagatagtggtgtataaggaggccgagaagagcccacagctgggactgggctgcctctccttcccgccgccccgggccggggccggccctctcccgagctcagagagtaaatatgagatcgcctctgtcccagtctccttacagacactgaaacctgttaccgacagagcaaacccgaaaatgggcataggaatgctggcatttagtcctgacagctacttcctggcgacaaggaacgacaacattcccaatgccgtctgggtctgggacattcagaagctgaggctgttcgcggtgctcgagcagctgtccccagtgcgcgcgtttcagtgggacccgcagcagccgcggctggccatctgcacgggaggcagcaggctctacctgtggtccccagcgggctgcatgtcggtgcaggtgcctggggaaggcgactttgcagtgctctctctgtgctggcatttaagcggagactcgatggccctcctcagcaaggatcacttctgcctctgcttcctggagacagaggcagtggtcggcacagcctgcagacagctgggcggccacacgtagcagcggtgcactaacgtgtgcagaaacagggctactctgtgtttccagtgtgggaaaaaacacagcttcaccaggaggttctccactgtggtggtctggattcagtgattgattctatttttctatagcaaagcatttttgtaaatatgtatggtataaaactgtagttttattatttaaaataaatacttgctgatttataaaaaaaaaaaaaaaaaa

SEQ ID NO: 164 - protein, 415 aa, Homo sapiens
  UNSURE (20)
  UNSURE (65)
MNFSEVFKLSSLLCKFSPDXKYLASCVQYRLVVRDVNTLQILQLYTCLDQIQHIEWSADSLFILXAMYKRGLVQVWSLEQPEWHCKIDEGSAGLVASCWSPDGRHILNTTEFHLRITVWSLCTKSVSYIKYPKACLQGITFTRDGRYMALAERRDCKDYVSIFVCSDWQLLRHFDTDTQDLTGIEWAPNGCVLAVWDTCLEVRILNHVTWKMITEFGHPAAINDPKIVVYKEAEKSPQLGLGCLSFPPPRAGAGPLPSSESKYEIASVPVSLQTLKPVTDRANPKMGIGMLAFSPDSYFLATRNDNIPNAVWVWDIQKLRLFAVLEQLSPVRAFQWDPQQPRLAICTGGSRLYLWSPAGCMSVQVPGEGDFAVLSLCWHLSGDSMALLSKDHFCLCFLETEAVVGTACRQLGGHT

SEQ ID NO: 165 - DNA, 3178 bp, Homo sapiens
  unsure (1653)
  unsure (1767)
atttcttttgccacttaaaattaactgtgggctactcagccagggtacagtgggagcctcaggaaggtcagaggcaacctcctcccctgttctatcaatagaaacccaacgttgaggcaattcctaaacagacgcacctcgtagcttgctgtatgtgtttattctttattgctttcagctttggggctgtaacaggtacaaatatttggtttccctatgatttatagagaagaagaagaaacccagctttctatcagagcactgcaagagaagagtcttacacctgccctcagtgggagatgagaatggtcattatgacttagagaatgctacacgtgtaggttgctggtgtgtcctgaatccacaggcataaagcactccccattttcctactgtaatgcagattctccggctcaaggtctagaatatttgatcctaagatcaagacatcatgcccttcgaatagtactgctctttgttttcaggagtcacgtgaacacacaactctcctatattcctcacgaacctcaggattgagcaaggtctttgtaatttttttggttcactttattgacctgggagcaaggtgctaattctgtggtcagtattcaatgtttttttcagtggagctttttctttgggccatatttgccttctaatacattcctgcaatatgtagtggtgatttcccttagcttcctcctactacctcttatactcatctccccaaattatttgcctcccttaaataagttttcctagaaggtaagctggtcaggcaatttgaaaaatattagatcccaagaaatctattccgtttgcattggacttctcggattccatgtgtttgcagcaggactacatcgaactctgatgtgccggattgtggcatgtctgcatgtctcatccatctattgtttttggtaactcagtttggaatttcagtgtctgtcttccctgggttgacattggaatcagcctctcctttgagcttattttaactcttgagcaacataacatagatttaatgtgaacagtttataccaaagggcagcctgtgcctgtttatggatcctctctgcctttgtacttgaagagcgcattttacatttccagtcctttcacagacaggagctccaaccttacgatggagaattaaacttgcttgtatttccactttgtggatgaggaactatgagaggtggagtgacttcctgggtccccgctgagacttagtgacagatcccagacaagaacttcatctctgactccaggtctagtcctcttccccctgtctcttgccaactccagccctgacaccgtgggcgtctcccctgagagcagatatatttcaattgtccaggccaaaagaggggcgaggcggcataaacacccaaattaggtggaggatccaaaagtcattttcatttggctgtggaatatgttttttgtatttcaatcagctaggggtgtgttcactgtttttggaaattcacagcgcttgagcctccataatgaagctgggctgcagagcacctggcacgtgctccaggctcccagctcccaacctaggacctctccctgccctccactctggttggtttgtggttccctcgaccgagggtttctagaatcagggaccttgtctaagtnttgtttgcccagagcccagcgaagtgtgtgatacaccttgggagtttaggagatcacaaaagggatgaaaacacctttagaaaacatttcattggtggggcgtggtggcttatncctgtgatcccagcctcctgagtagctgggatcacaggcgtacaccaccacacccagctaagtttttgtacttttagtggagacggggtttcaccatgttggccaggctggtcacggactcctgacctcagatgatctgcccgcctgagcctccgaaagtgctgggattacaggcgtgagccactgcgcccggcatggagctgctattgatgggtgagctccacagcttttgcagaagcagaggatatgacttgagagcagtgctgtcacctctcagcatgtccccaagcccaactggggcctcctggagatgcctcagtcggcactggccccaagggaatcgtggggaacagttgcacaatttgcaagtttctgagtgcagcttttcccatccttgggatcagcagataagttgtaaacacagggaggtactgcttattggatatacttttcataagtaggacagaattcttttgggactctagagttgggaactaccacttactagcggcgtggctgaggcagtcttcctcctctgtggctcaggcccttcatctgtgaaatggggtcacagcatctgcctctcagggtcactgtgaggtgtcgatgtgagcaaggcctgaggcttggcaagaagtcaatgtctgcaactcagcagggagcaatggcaggggcagtcaggggtcggctcggataggggtgggtgggctcctgaggttggaagggtaggaattacagagctcttgttactattgttgttactgtttttaaagatacgatatttcagataattcaggagcacgtaaggatgaaacttaggataacctaaaatcacacaacctagagagaagcgcatttttgtcttcccccattttctaggcaaaaatgaaagtactttgtcctcttgaaaaacaattctctaatgaatatcctatgttacatagaggcctgtgtaatgcatttgtgtggccacatggtgtcaatttcatgaatatacaataatattattaattccctgctgaggacattaactggtttccaaggctgcttgttgtttttgctactacaaataatgcattgatgataaatacttttacatacatggttgtatgtttatctgaactattttcaccaatatattcacctagtgtgtatggaagtgtccatttttgtcatacccctggtaaccctgtgatattatttttaaacattttgctaatggatctctgttcttgtttgaatgtatttaatttccagcagaatgagccccattccttattttgattggccatttatcatgtacatatggtgaaatgcctattcgtgacttagccaatgttgtttctttttcttactgattactacagtacatttttatatgaaaaaaaaaaaaaaaaaaaaaaaaaaaaaa

SEQ ID NO: 166 - protein, 67 aa, Homo sapiens [sequence omitted]

SEQ ID NO: 167 - DNA, 2401 bp, Homo sapiens
cgcatcctcagccaccgtcgcagctgcctccgccaccaccgccgcctcctcttccttggccaccccagaactgggcagcagcctcaagaagaagaagcggctctcccagtcagatgaggatgtcattaggctaataggacagcacttgaatggcttagggctcaaccagactgttgatctcctcatgcaagagtcaggatgtcgtttagaacatccttctgctaccaaattccgaaatcatgtcatggaaggagactgggataaggcagaaaatgacctgaatgaactaaagcctttagtgcattctcctcatgctattgtgaggatgaagtttttgctgctgcagcagaagtacctagaatacctggaggatggcaaggtcctggaggcacttcaagttctacgctgtgaattgacgccgctgaaatacaatacagagcgcattcatgttcttagtgggtatctgatgtgtagccatgcagaagacctacgtgcaaaagcagaatgggaaggcaaagggacagcttcccgatctaaactattggataaacttcagacctatttaccaccatcagtgatgcttcccccacggcgtttacagactctcctgcggcaggcggtggaactacaaagggatcggtgcctatatcacaataccaaacttgataataatctagattctgtgtctctgcttatagaccatgtttgtagtaggaggcagttcccatgttatacgcagcagatacttacggagcattgtaatgaagtgtggttctgtaaattctctaatgatggcactaaactagcaacaggatcaaaagatacaacagttatcatatggcaagttgatccggatacacacctgctaaaactgcttaaaacattagaaggacatgcttatggcgtttcttatattgcatggagtccagatgacaactatcttgttgcttgtggcccagatgactgctctgagctttggctttggaatgtacaaacaggagaactaaggacaaaaatgagccagtctcatgaagacagtttgacaagtgtggcttggaatccagatgggaagcgctttgtgactggaggtcagcgtgggcagttctatcagtgtgacttagatggtaatctccttgactcctgggaaggggtaagagtgcaatgcctttggtgcttgagtgatggaaagactgttctggcatcagatacacaccagcgaattcggggctataacttcgaggaccttacagataggaacatagtacaagaagatcatcctattatgtcttttactatttcaaaaaatggccgattagctttgttaaatgtagcaactcagggagttcatttatgggacttgcaagacagagttttagtaagaaagtatcaaggtgttacacaagggttttatacaattcattcatgttttggaggccataatgaagacttcatcgctagtggcagtgaagatcacaaggtttacatctggcacaaacgtagtgaactgccaattgcggagctgacagggcacacacgtacagtaaactgtgtgagctggaacccacagattccatccatgatggccagcgcctcagatgatggcactgttagaatatggggaccagcaccttttatagaccaccagaatattgaagaggaatgcagtagcatggatagttgatggtgaatttggagcagacgacttctgtttaacttaaaattagtcgtattttaatggcttgggatttggtgcaaacaaacatgattgatagctggacagacatgctcgtcatgaaaaaagaaccatttctgaagcccgattggggccaaacatttacaccttgcttcatagtaaccagttgagatgaagcacgtcgttagaacgttgttggacaccatgttgaattattcccccatcggttgtgaagaactgtgctacattcaggcttacccattgaactcagtatatatatttttttccttcctgtcttttgtctggcaggataccattcttgttgctcttctgtgtaatgaagtttaaatgcttgtttggaaaactttatttaacagtttagaaggcttgatagaaagagtgcattagtctgaagagtatacattggataggaaagaatttccttcttttgtttctccaaatctttccgccttatttagcttgagatctttgcagcttggttcatggattctagccttgcccgttgcgcagtatatactgatccagatgataaaccagtgaactatgtcaaaagcactctcaatattacatttgacaaaaagttttgtacttttcacatagcttgttgccccgtaaaagggttaacagcacaattttttaaaaataaattaagaagtatttataggattaaaaaaaaaaaaaaaaaaa

SEQ ID NO: 168 - protein, 498 aa, Homo sapiens
MQESGCRLEHPSATKFRNHVMEGDWDKAENDLNELKPLVHSPHAIVRMKFLLLQQKYLEYLEDGKVLEALQVLRCELTPLKYNTERIHVLSGYLMCSHAEDLRAKAEWEGKGTASRSKLLDKLQTYLPPSVMLPPRRLQTLLRQAVELQRDRCLYHNTKLDNNLDSVSLLIDHVCSRRQFPCYTQQILTEHCNEVWFCKFSNDGTKLATGSKDTTVIIWQVDPDTHLLKLLKTLEGHAYGVSYIAWSPDDNYLVACGPDDCSELWLWNVQTGELRTKMSQSHEDSLTSVAWNPDGKRFVTGGQRGQFYQCDLDGNLLDSWEGVRVQCLWCLSDGKTVLASDTHQRIRGYNFEDLTDRNIVQEDHPIMSFTISKNGRLALLNVATQGVHLWDLQDRVLVRKYQGVTQGFYTIHSCFGGHNEDFIASGSEDHKVYIWHKRSELPIAELTGHTRTVNCVSWNPQIPSMMASASDDGTVRIWGPAPFIDHQNIEEECSSMDS

SEQ ID NO: 169 - DNA, 1110 bp, Homo sapiens
ggtgcgggagccgctctccgccggtcggtccccgcgcggctgagcccaggccgccagcgccgcggccccgtgcggtgtccctgagctcctgctccccgccgggctgctccgagcaacggtgcttcggagctccaaactcgggctgccggggcaagtgtcttcatgaacccagaggatgtccgggaagcactacaagggtcctgaagtcagttgttgcatcaaatacttcatatttggcttcaatgtcatattttggtttttgggaataacatttcttggaattggactgtgggcatggaatgaaaaaggagttctgtccaacatctcttccatcaccgatctcggcggctttgacccagtttggctcttccttgtggtgggaggagtgatgttcattttgggatttgcagggtgcattggagcgctacgggaaaacactttccttctcaagtttttttctgtgttcctgggaattattttcttcctggagctcactgccggagttctagcatttgttttcaaagactggatcaaagaccagctgtatttctttataaacaacaacatcagagcatatcgggatgacattgatttgcaaaacctcatagacttcacccaggaatatattccaatgcaagtcgagagcgatgtggcgttccattctcctgctgcactaaagatcccgcagaagatgtcatcaacactcagtgtggctatgatgccaggcaaaaaccagaagttgaccagcagattgtaatctacacgaaaggctgtgtgccccagtttgagaagtggttgcaggacaatttaaccwtcgttgctggtattttcataggcattgcattgctgcagatatttgggatmtgcctggcccagaatttggttagcgatatcgawgctgtcagggcgagctggtagaccccctgcaaccgctgctgcaagacactggacagacccagctttcgggaccctcccgcgtgccgaactgatcttcgagctgcatggacctaatcacagatgcagcctgcagtctcgcctaatggagctgccattaggggagtgtaaaactgggaaatgctgctcactgacagaattaaaaaaaaaaaaaaa

SEQ ID NO: 170 - protein, 193 aa, Homo sapiens
MSGKHYKGPEVSCCIKYFIFGFNVIFWFLGITFLGIGLWAWNEKGVLSNISSITDLGGFDPVWLFLVVGGVMFILGFAGCIGALRENTFLLKFFSVFLGIIFFLELTAGVLAFVFKDWIKDQLYFFINNNIRAYRDDIDLQNLIDFTQEYIPMQVESDVAFHSPAALKIPQKMSSTLSVAMMPGKNQKLTSRL

SEQ ID NO: 171 - DNA, 1621 bp, Homo sapiens
ctttaaaatgtggctaatgcctgccttagggaaccgttgtgaggattaagtgagacatggtatataaaacgacctccttctggcataaacttgaggtggaagataccttgaggatgcttgaaggtctgctaggcagcttcacagccttttctttcctcttctctatcagaggtctctttggaagcaataatgatgactataacaagaacttatcttgctttgcaagattcttccgccgtcagagtttctgatttattttctggggttccatgtatgccagggagaaagagagagcgcgaaagagagaggatgtctctctcagactggcacctggcggtgaagctggctgaccagccacttactccaaagtctattcttcggttgccagagacagaactgggagaatactcgctagggggctatagtatttcatttctgaagcagcttattgctggcaaactccaggagtctgttccagaccctgagctgattgatctgatctactgtggtcggaagctaaaagatgaccagacacttgacttctatggcattcaacctgggtccactgtccatgttctgcgaaagtcctggcctgaacctgatcagaaaccggaacctgtggacaaagtggctgccatgagagagttccgggtgttgcacactgccctgcacagcagctcctcttacagggaggcggtctttaagatgctcagcaataaggagtctctggatcagatcattgtggccaccccaggcctcagcagtgaccctattgctcttggggttctccaggacaaggacctcttctctgtcttcgctgatcccaatatgcttgatacgttggtgcctgctcacccagccctcgtcaatgccattgtcctggttctgcactccgtagcaggcagtgccccaatgcctgggactgactcctcttcccggagcatgccctccagctcataccgggatatgccaggtggcttcctgtttgaagggctctcagatgatgaggatgactttcacccaaacaccaggtccacaccctctagcagtactcccagctcccgcccagcctccctggggtacagtggagctgctgggccccggcccatcacccagagtgagctggccaccgccttggccctggccagcactccggagagcagctctcacacaccgactcctggcacccagggtcattcctcagggacctcaccaatgtcctctggtgtccagtcagggacgcccatcaccaatgatctcttcagccaagccctacagcatgcccttcaggcctctgggcagcccagccttcagagccagtggcagccccagctgcagcagctacgtgacatgggcatccaggacgatgagctgagcctgcgggccctgcaggccaccggtggggacatccaagcagccctggagctcatctttgctggaggagccccatgaactccctgcttcccctgaacccccagcaagttgcagaggctactgcccttgggaggcactcatgaaggtgcctccatctctcccttccccaatatacctgatggtcaactctaaaaaaaaaaaaaaaaaaaaaaaaaaaaaaaaaaaaaaaaaaaaaaaaaaaaaaaaa

SEQ ID NO: 172 - protein, 420 aa, Homo sapiens
MMTITRTYLALQDSSAVRVSDLFSGVPCMPGRKRERERERMSLSDWHLAVKLADQPLTPKSILRLPETELGEYSLGGYSISFLKQLIAGKLQESVPDPELIDLIYCGRKLKDDQTLDFYGIQPGSTVHVLRKSWPEPDQKPEPVDKVAAMREFRVLHTALHSSSSYREAVFKMLSNKESLDQIIVATPGLSSDPIALGVLQDKDLFSVFADPNMLDTLVPAHPALVNAIVLVLHSVAGSAPMPGTDSSSRSMPSSSYRDMPGGFLFEGLSDDEDDFHPNTRSTPSSSTPSSRPASLGYSGAAGPRPITQSELATALALASTPESSSHTPTPGTQGHSSGTSPMSSGVQSGTPITNDLFSQALQHALQASGQPSLQSQWQPQLQQLRDMGIQDDELSLRALQATGGDIQAALELIFAGGAP

SEQ ID NO: 173 - DNA, 1534 bp, Homo sapiens
aaaccctggtgctccagacaaagatcttagtcgggactagccggccaaggatgaagcctcacttcagaaacacagtggagcgaatgtatcgagacacattctcctacaacttttataatagacccatcctttctcgtcggaataccgtctggctgtgctacgaagtgaaaacaaagggtccctcaaggccccctttggacgcaaagatctttcgaggccaggtgtattccgaacttaagtaccacccagagatgagattcttccactggttcagcaagtggaggaagctgcatcgtgaccaggagtatgaggtcacctggtacatatcctggagcccctgcacaaagtgtacaagggatatggccacgttcctggccgaggacccgaaggttaccctgaccatcttcgttgcccgcctctactacttctgggacccagattaccaggaggcgcttcgcagcctgtgtcagaaaagagacggtccgcgtgccaccatgaagatcatgaattatgacgaatttcagcactgttggagcaagttcgtgtacagccaaagagagctatttgagccttggaataatctgcctaaatattatatattactgcacatcatgctgggggagattctcagacactcgatggatccacccacattcactttcaactttaacaatgaaccttgggtcagaggacggcatgagacttacctgtgttatgaggtggagcgcatgcacaatgacacctgggtcctgctgaaccagcgcaggggctttctatgcaaccaggctccacataaacacggtttccttgaaggccgccatgcagagctgtgcttcctggacgtgattcccttttggaagctggacctggaccaggactacagggttacctgcttcacctcctggagcccctgcttcagctgtgcccaggaaatggctaaattcatttcaaaaaacaaacacgtgagcctgtgcatcttcactgcccgcatctatgatgatcaaggaagatgtcaggaggggctgcgcaccctggccgaggctggggccaaaatttcaataatgacatacagtgaatttaagcactgctgggacacctttgtggaccaccagggatgtcccttccagccctgggatggactagatgagcacagccaagacctgagtgggaggctgcgggccattctccagaatcaggaaaactgaaggatgggcctcagtctctaaggaaggcagagacctgggttgagcctcagaataaaagatcttcttccaagaaatgcaaacaggctgttcaccaccatctccagctgatcacagacaccagcaaagcaatgcactcctgaccaagtagattcttttaaaaattagagtgcattactttgaatcaaaaatttatttatatttcaagaataaagtactaagattgtgctcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 174 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 175 - DNA, 3005 bp, Homo sapiens
  unsure (1407)
aaagaagttgtacgaaggtcaaagaaattgtctgttccagcctcagtggtgtcgaggataatgggaagaggaggatgcaacatcactgcaatacrkgatgttactggtgcccatattgatgtggataaacaaaaagataagaatggcgagagaatgatcacaataaggggtggcacagaatcaacaagatatgcagttcaactaatcaatgcactcattcaagatcctgctaaggaactggaagacttgattcctaaaaatcatatcagaacacctgccagcaccaaatcaattcatgctaacttctcatctggagtaggtaccacagcagcttccagtaaaaatgcatttcctttgggtgctccaactcttgtaacttcacaggcaacaacgttatctacgttccagcccgctaataaacttaataagaatgttccaacaaatgtacgttcttctttcccagtttctctacccttagcttatcctcaccctcattttgccctgctggctgctcaaactatgcaacagattcggcatcctcgcttacccatggcccagtttggaggaaccttctcaccttctcctaacacatggggaccattcccagtgagacctgtgaatcctggcaacacaaatagctctccaaagcataataacacaagccgtctacctaaccagaacgggactgttttaccctcagagtctgctggactagctactgccagttgtcctatcactgtctcttctgtagttgctgccagtcagcaactgtgtgtcactaatacccggactccttcatcagtcagaaagcagttgtttgcctgtgtgcctaagacaagtcctccagcaacagtgatttcttctgtgacaagcacttgtagttccctgccttctgtctcctctgcacctatcactagcgggcaagctcccaccacatttctacctgcaagtacttctcaagcacagctttcttcacaaaagatggagtctttctctgctgtgccacccaccaaagagaaagtgtccacacaggaccagcccatggcaaacctatgtaccccatcttcaactgcaaacagttgcagtagctctgccagcaacaccccgggagctccagaaactcacccatccagtagtcccactcctacttccagtaacacacaagaggaggcacagccatccagtgtgtctgatttaagtcctatgtcaatgccttttgcatctaactcagaacctgctccattgactttgacatcacccagaatggttgctgctgataatcaggacaccagtaatttacctcagttagctgtaccagcacctcgagtttctcatcgaatgcagcccagaggttctttttactccatggtaccaaatgcaactattcaccaggatccccagtctatntttgttacgaatccagttactttaacaccacctcaaggcccaccagctgcagtgcagctttcttcagctgtgaacattatgaatggttctcagatgcacataaacccagcaaataagtctttgccacctacatttggcccagccacacttttcaatcacttcagcagtctttttgatagtagtcaggtgccagctaaccagggctggggagatggtccactgtcctcacgagttgctacagatgcctctttcactgttcagtcagcgttcctgggtaactcagtgcttggacacttggaaaacatgcaccctgataactcaaaggcacctggcttcagaccaccttcccagcgagtttctactagtccagttgggttaccatccattgacccatcaggcagctccccatcttcctcttctgctcctctggcaagtttttccggcataccaggaacaagggttttcctgcaagggccagctcctgttgggactcctagtttcaacagacaacatttttctccccatccttggacaagcgcctcaaactcatccacttctgccccaccaacgttgggccaaccaaaaggagtcagtgccagtcaagatcgaaagatacctcccccaattggaacagagagactggcccgaattcggcaaggagggtctgttgcacaagccccggcggggaccagttttgtcgctcccgttggacacagtggaatctggtcatttggtgtcaatgctgtgtcagaaggcttatcaggttggtcgcaatctgtgatggggaaccatccaatgcatcaacaattatcagacccaagcacattctcccaacatcagccaatggagagagatgattctggaatggtagccccctctaacatttttcatcagcctatggcaagtggttttgtggatttttctaaaggtctgccaatttccatgtatggaggcaccataataccctctcatcctcagcttgctgatgttccaggaggccctctgtttaatggacttcacaatccagatcctgcttggaaccctatgataaaagttatccaaaattcaactgaatgcactgatgcccagcagatttggcctggcacgtgggcacctcatattggaaacatgcatctcaaatatgtcaactaagttagaaggtctttactctttagccttgtttaagaaacctatgaccttggaagaaccatggggatttttttttaatgtgcctaagaaattttctctgaggctttagcaatggaaatttgattgcccattgtataagaacaaattgatttcctatccacctgattatgttctctggttagtttagccattttgaacttaagatcatatgaccttagtgcttttggctaaacatacagaatactacttgtatgcagaagagaattagttgattacatgtttcaaccttttagggtgataaatacatgtataattgtttacatacttaaaaggaaaaagttgagtaaatttcttgtcatatagtggctctacgtaatgtagcctgtattaatgtgaaatatttaccagaatattcaataaaaagatgaacagtcaaaaaaaaaaaaaaa

SEQ ID NO: 176 - protein, 832 aa, Homo sapiens
  UNSURE (12)
  UNSURE (449)
MGRGGCNITAIXDVTGAHIDVDKQKDKNGERMITIRGGTESTRYAVQLINALIQDPAKELEDLIPKNHIRTPASTKSIHANFSSGVGTTAASSKNAFPLGAPTLVTSQATTLSTFQPANKLNKNVPTNVRSSFPVSLPLAYPHPHFALLAAQTMQQIRHPRLPMAQFGGTFSPSPNTWGPFPVRPVNPGNTNSSPKHNNTSRLPNQNGTVLPSESAGLATASCPITVSSVVAASQQLCVTNTRTPSSVRKQLFACVPKTSPPATVISSVTSTCSSLPSVSSAPITSGQAPTTFLPASTSQAQLSSQKMESFSAVPPTKEKVSTQDQPMANLCTPSSTANSCSSSASNTPGAPETHPSSSPTPTSSNTQEEAQPSSVSDLSPMSMPFASNSEPAPLTLTSPRMVAADNQDTSNLPQLAVPAPRVSHRMQPRGSFYSMVPNATIHQDPQSXFVTNPVTLTPPQGPPAAVQLSSAVNIMNGSQMHINPANKSLPPTFGPATLFNHFSSLFDSSQVPANQGWGDGPLSSRVATDASFTVQSAFLGNSVLGHLENMHPDNSKAPGFRPPSQRVSTSPVGLPSIDPSGSSPSSSSAPLASFSGIPGTRVFLQGPAPVGTPSFNRQHFSPHPWTSASNSSTSAPPTLGQPKGVSASQDRKIPPPIGTERLARIRQGGSVAQAPAGTSFVAPVGHSGIWSFGVNAVSEGLSGWSQSVMGNHPMHQQLSDPSTFSQHQPMERDDSGMVAPSNIFHQPMASGFVDFSKGLPISMYGGTIIPSHPQLADVPGGPLFNGLHNPDPAWNPMIKVIQNSTECTDAQQIWPGTWAPHIGNMHLKYVN

SEQ ID NO: 177 - DNA, 1561 bp, Homo sapiens
  unsure (1150)
gagaaggaagggaagccggaaggggcgcgagtgaagcaaagcgaggacagacagctcccagagggcgaggggtgcgtgtgcgtccgcttctcacctcaggtctcccttcggccccgctgccctccctcgcggctgggtgacagctgggtccggtccgtcgcgggctgcctggggtgcgaggatcgcgcaccccgtcttcgcgcgctgtgcctgccgccccgccccctcgtcccgcccgtcccgtcgcgtcgcgtcccgtcccctcgggtgctgccagccgggtgctgatgcgagtcggtggcagcgaggacattttctgactccctggcccctgacacggctgcactttccatcccgtcgcggggccggccgctactccggccccaggatgcagaatgtgattaatactgtgaagggaaaggcactggaagtggctgagtacctgaccccggtcctcaaggaatcaaagtttaaggaaacaggtgtaattaccccagaagagtttgtggcagctggagatcacctagtccaccactgtccaacatggcaatgggctacaggggaagaattgaaagtgaaggcatacctaccaacaggcaaacaatttttggtaaccaaaaatgtgccgtgctataagcggtgcaaacagatggaatattcagatgaattggaagctatcagtgaagaagatgatggtgatggcggatgggtagatacatatcacaacacaggtattacaggaataacggaagccgttaaagagatcacactggaaaataaggacaatataaggcttcaagattgctcagcactatgtgaagaggaagaagatgaagatgaaggagaagctgcagatatggaagaatatgaagagagtggattgttggaaacagatgaggctaccctagatacaaggaaaatagtagaagcttgtaaagccaaaactgatgctggcggtgaagatgctattttgcaaaccagaacttatgacctttacatcacttatgataaatattaccagactccacgattatggttgtttggctatgatgagcaacggcagcctttaacagttgagcacatgtatgaagacatcagtcaggatcatgtgaagaaaacagtgaccattgaaaatcaccctcatctgccnccacctcccatgtgttcagttcacccatgcaggcatgctgaggtgatgaagaaaatcattgagactgttgcagaaggagggggagaacttggagttcatatgtatcttcttattttcttgaaatttgtacaagctgtcattccaacaatagaatatgactacacaagacacttcacaatgtaatgaagagagcataaaatctatcctaattattggttctgatttttaaagaattaacccatagatgtgaccattgaccatattcatcaatatatacagtttctctaataagggacttatatgtttatgcattaaataaaaatatgttccactaccagccttatttgtttaataaaaatcagtgcaaagagaaaaaaaaaaaaaaaaaaaaaaaaaaaaaaaaaaaaaaaa

SEQ ID NO: 178 - protein, 314 aa, Homo sapiens
MQNVINTVKGKALEVAEYLTPVLKESKFKETGVITPEEFVAAGDHLVHHCPTWQWATGEELKVKAYLPTGKQFLVTKNVPCYKRCKQMEYSDELEAISEEDDGDGGWVDTYHNTGITGITEAVKEITLENKDNIRLQDCSALCEEEEDEDEGEAADMEEYEESGLLETDEATLDTRKIVEACKAKTDAGGEDAILQTRTYDLYITYDKYYQTPRLWLFGYDEQRQPLTVEHMYEDISQDHVKKTVTIENHPHLPPPPMCSVHPCRHAEVMKKIIETVAEGGGELGVHMYLLIFLKFVQAVIPTIEYDYTRHFTM

SEQ ID NO: 179 - DNA, 2379 bp, Homo sapiens
atttagttacacatagacataactcttcaaccttaactatggcaatacatttgtgctttaactgttacatagcagtatcaccacttaccaggatccaaatcgaaataataaaagctgtctccatagtttaaaatcgaatagtgccatcatcacagtatattagtcaaatagaagcttcatcagaaatgtatcccacatagagttttaagacttggattctcttctgcccttgttaatctccaactaattactacagattgacacgtttttaattagctgtcctttgtaagaagtcaggaaatctgatgctgtgtccaaaattatgcactgtttgttgaagtagaaccagaaatcctgacctcctgttaaatgacatcagtttccccctctgagcaacagactgcttgtcttgctaggagaggaggatggggggctgagcactcaggctgtccattgaaaccccttgtccatgaatagggtcatactcctaagactgatggggtgttgatcttctaggacatcacttgtttattcagtgccccaaacacagatttctcttctagcactttagaattgatccttgaagtctctcctggttcattcaaatacaagctgtgtgagtctggtggttttctgtgattggtctaatgtgagctctttgaacagacagatctgacagtggaatgactctcccctgcttctggcataactgctttgcctctgtctagtgtccaagcatcttagctgttcaagaggagagggcagcataacttcctgaccactggtgtcagatatcagagcattctggactcctgagaggcagtggcctcttgagtgaacaggggaggccagtagatgccccagatccagagccgtggctgcaaatccagcaggaataaggagggacaaccacagcctcctcatccatgtgtcatttccaagggtttgccttgtgtctcagctcattctgggcagcacgtttgtcttctgtccctagagatttgaaggattttggactcttgtgaatgggtgactggacttggctttacagagttgggtgcttttttctctctgcaattacctgtcatagcattttgtgctcaccacgaaggatggtctctgccttctcttgtcggtgtatgccatctgaacctaggaacacaaagtatattggcctcaaacgggagacccagggttgccagttttccgtgggccttcccctcccttgaaatgtctttaattacctccccttcatcgtcaggccacgtgtgacttctgttcttagcactgccagggtcattgacttccatctaagcttgcatcaggaagatgttccttctgtgatcattggtactgaagccagaaaagctctcattcaggaactctgaagagcaaaaagggacaaacactaactgctgagctgggccatttgatctcctttcaccttgcattgctgtcacagcaccttgtatgatggcaggacaggctccagcagagagaactgcacagtgaccactgtatttttcacgctcttccagggatccctgtcccccgacattgaagagatctcattcaggccagagacacagagaccacatagcccagtgattaaaccccggtttcactctggccccaggagtggagcctggccactcctgtttggttctcactgggaggcccactggccttggatcatctcctcatgcacacccggagttttacctgcttgcttgctttcctggactgctgtttgcaagaaagtaactaaaacatgaaaagtaaacctccagcttccacagtatattacctgccgttgcatgcatttgaaagttagcctcctcccttgccaccgtcttggtggcagtagcgatgcaagaatgatgggagctttccgagagcgttcagtgtttcactgaagacaggacccatagccttcatttctggctctgtgtctcctctggcatatggacacatttcctggcatttgcctgagtctacaccactttttgagaacctgaaatagaagggaatcttctgtggcccacagtctccatattggccctagaagactggcctggcggaggaatttgcgttggcttgctttcaggggttagctacaagattcagctttatatctctgttgcttcttggccagtgtagtcaataagggtcttctttaacatctaagatagaggtttggttggccgggcgtggtcgctwactcctgtaatcccagcactttgggaggcccagtgaggtgggagaattgcttgaacccaggaggcagaggttgcagtgagctgagattgcaccattgcattccagcctgggtaacagagtgagactcttgtctcaaaaaaaaaaaaaaaaaaaaaaaaa

SEQ ID NO: 180 - protein, 67 aa, Homo sapiens
MGDWTWLYRVGCFFLSAITCHSILCSPRRMVSAFSCRCMPSEPRNTKYIGLKRETQGCQFSVGLPLP

SEQ ID NO: 181 - DNA, 1607 bp, Homo sapiens
atacaagtcaagatgctacccatgtagacacactgtatttttaaggtgggcaagtgcgattaacgatgaaccattttaaaggggaggttatttgaaacctctaatttgattattgggaggattttcatgctttctttagtatttattaccatcataccgattcaaactattttattgtctaatacattagcattttgtattttgatggaaattgttacagaatttaaagatttgatgaaataagatgtagcagattttttgtagcaagtttctggtaaaagggttttttgcaagtctcaggttcttgctgcactatttttttttaaatatttattccagttattctaattcagaagcattcttttcaagtaacagcagcacttgtgaaaggaaaaaaaaatgcacatgtttcttagtaggttactaaatttgtacaattaattaagattttagccatcagtgagtttgaaaagggaaatgtatttattttcagcattaaaatgcttccaaaagatcaagttgcttttgtttgtttgtttttttaaccgtaatgtagatggagaaattggaggcaacctcagtataggaactgccactttgagcagtttaggtcttaaagagaaagtcaatctaatgccaaggggagaacaatgagctgaaattgtaccaactcctctggccctccttccctcaattaaaaaaacacacttaccagttttgcttattttacagatatctggtggttctatagtttaaagcagcttgtgaaattaaaaaagtggactcaattttgtttacctttctgtaagtttttcatttttgctgtatagcattggcaaaaatatgtacaaattgacctctgttcttatttcctattgtgagcattataaagataagctcctatgtaaaaccttgctctcagatgagtaaaatatgtatcacagcatagctcagcaataattcatgctcagctgtggggaccctgggggctttttgaagatgatggaaccgcactagggttgaaactgatggctgtggagttaattgtgttttcgagcttgaatctcacctgtgattttttttttttaatgttgtttcatgacttgatttttctcataagccaatgtatttgtaggtttactggattttatttttagggagtgggtaatttcttcccttttttgattaagttggttcagctatggtgctattcagtaggtatcttcagtgtcaggtcccgtagctgaatgccattgttattataattattatttgtaatcacattgtaagcttgaatttgggcttgwacctgcatcttttgtattctgtacatctggttacttagactttgggagtccaatttggtttcagtcatgtatgtctactttgtagtttaagtagacttcatcaactatggtctattttgggtttgtagttttaatttagaattgtgttaaattgatgttttgcatttgacttcatttgacattagttgaagtaaattatttaatttttgaattctggaatttgaacatttactgtaatttgtaatataactgctgtgaaatacttgaataaagatgacaagaaaaaaaaaaaaaaaaaaa

SEQ ID NO: 182 - protein, 58 aa, Homo sapiens [sequence omitted]

SEQ ID NO: 183 - DNA, 2695 bp, Homo sapiens
gaacagagtagtagccaggcaatgttctcataataaacagaaaaggaaaagaaactccaatgtggaaaccatctcaaacctctgtgtgaagtctaccaattttctgttaatcaaagcaagctatgtgagtgtactcagagtccaggggcaaggtagtcaccctgtgtgtggtgggaaaatactgcaagattatatgtcaaataatgggatactcaggaatatttacaaaaatgttgaatattttaatgaaataacaaatatttagacattcaatagacttgagagtaactttaccaagggtctaagtatgagagatatgtttaatatatttttatgggctgaaaaccctgagtgggaaaataggactaatttcaccaggatgacctcctggaaatgcattttccattttggaaattattttaaaagttcattttttctggatgggtatgtgtatgtgtgtgtgtctgtcyaygtgtgtatgttttatgagcttgttaacactaatgtcatacaaaagtactggttagcaggaataagattttaaggtgtattggcattcccatggttcccaagaaaattttagatgactttgattaaaaagtttggattttgtctatttaaatctagcataaaaattggtcatggtgatgatcctagttatgactaatctccctttaagatttaggcatttactgtgtgaaatatgtggcacattttccataacaaacagctaaagttactgaacacaaattatggaaaggtgaaatgaggaaaacattgcaaaacactgaaagagaatatgtctttatttgcatgctggcaaatgaaaattccggtttcacttctacttcagtatctaacaagtctctaacaagaacagacattgaatgaatgaattaagttgagctgtttgaaaattagaatgttttccataaatacattattgaactatcaattagcataaactgctactttcttgtttgacactggtcacagtatttgaaagtaaaaagaatgttactgcacattcagaaatcaggtccacataaaatttaaggtcaggatattaaaggatcacagccagtgctgttaggccttcatttattctatctttttgtctgttcagacatgataacttttctacccatcattttttccattctagtagtggttacatttgttcttgggaattttgctaatggcttcatagtgttggtaaattccattgagtgggtcaagagacaaaagatctcctttgctgaccaaattctcactgctctggcagtctccagagttggtttgctctgggtaatattatwacattggtatgcaactgttttgaatccaggttcatatagtttaggagtaagaattactactattaatgcctgggctgtaaccaaccatttcagcatctgggttgctactagcctcagcatattttatttgctcaagattgccaatttctccaactttatttttcttcacttaaaaaggagaattaagagtgtcattccagtgatactattggggtctttgttatttttggtttgtcatcttgttgtggtaaacatggatgagagtatgtggacaaaagaatatgaaggaaacgtgagttgggagatcaaattgagtgatccgacgcacctttcagatatgactgtaaccacgcttgcaaacttaataccctttactctgtccctgttatcttttctgctcttaatctgttctttgtgtaaacatctcaagaagatgcagttccatggcaaaggatctccagattccaacaccaaggtccacataaaagctttgcaaacggtgacctccttcctcttgttatttgctgtttactttctgtccctaatcacatcgatttggaattttaggaggaggctgtagaacgaacctgtcctcatgctcagccaaactactgcaattatatacccttcatttcattcattcatcctaatttggggaagcaagaagctgaaacagacctttcttttgattttgtgtcagattaagtgctgagtaaaagacctgaaactctcaaatttctagattcacaagtgggacatcgtgtgtctccaagagaaaacaaactgatgttgtctggaacattttatactttccactggtttttctgtattgtatgtttttgagtaatttccaaaagtatatctagaaaagtcttttaccctaaagttagtctaaaaaggtatctatatakgcatgtgtatggtgtatatgaaacacttaagagagagtggcaataacataatcatttttwacaaactgccaaattatagaaaatattgtaagaaatttttcagaatcatgaagccatgtgtattcacaatacagttcatattatcatgtttcatttgaaaaatttatgatctctatttataattgttaagaacttacagcttatttcacaaaatcattgctcttttccactgttatttgtaccatacgtatgtaccatagtgtgcttaaacgtgattatttgaacgtctagttttttggatggtatgcgcattctaatctaaatcaataatgaagttttatctttggggtagtttttgttgcataatgaattctaattttatgtttaatttaaagcaaacaattattgttagaaaactatgcacaaaataaaattcaaggatgaaaaataaaaaaaaaaaaaaaa

SEQ ID NO: 184 - protein, 256 aa, Homo sapiens
  UNSURE (64)
MITFLPIIFSILVVVTFVLGNFANGFIVLVNSIEWVKRQKISFADQILTALAVSRVGLLWVILXHWYATVLNPGSYSLGVRITTINAWAVTNHFSIWVATSLSIFYLLKIANFSNFIFLHLKRRIKSVIPVILLGSLLFLVCHLVVVNMDESMWTKEYEGNVSWEIKLSDPTHLSDMTVTTLANLIPFTLSLLSFLLLICSLCKHLKKMQFHGKGSPDSNTKVHIKALQTVTSFLLLFAVYFLSLITSIWNFRRRL

SEQ ID NO: 185 - DNA, 1111 bp, Homo sapiens
gccgagcgccgccgccgaagcttccgtctcgctcgctcgcgcagcggcggcagcagaggtcgcgcacagatgcgggttagactggcggggggaggaggcggaggagggaaggaagctgcatgcatgagacccacagactcttgcaagctggatgccctctgtggatgaaagatgtatcatggaatgaacccgagcaatggagatggatttctagagcagcagcagcagcagcagcaacctcagtccccccagagactcttggccgtgatcctgtggtttcagctggcgctgtgcttcggccctgcacagctcacgggcgggttcgatgaccttcaagtgtgtgctgaccccggcattcccgagaatggcttcaggacccccagcggaggggttttctttgaaggctctgtagcccgatttcactgccaagacggattcaagctgaagggcgctacaaagagactgtgtttgaagcattttaatggaaccctaggctggatcccaagtgataattccatctgtgtgcaagaagattgccgtatccctcaaatcgaagatgctgagattcataacaagacatatagacatggagagaagctaatcatcacttgtcatgaaggattcaagatccggtaccccgacctacacaatatggtttcattatgtcgcgatgatggaacgtggaataatctgcccatctgtcaaggctgcctgagacctctagcctcttctaatggctatgtaaacatctctgagctccagacctccttcccggtggggactgtgatctcctatcgctgctttcccggatttaaacttgatgggtctgcgtatcttgagtgcttacaaaaccttatctggtcgtccagcccaccccggtgccttgctctggaaggaggaagacctgaacatcttttccctgtcctttatttcccacacatcaggttggcagctgctgtgctttatttttgccctgtgttaaagtcctctcccaccccagcacctacctgttcctcaactagcaccaccacatctctgttctaaatgttgttctcctgcaataaaggacgtttgaatttaaaaaaaaaaaaaaaaaaaaaaaaaaaaaaaa

SEQ ID NO: 186 - protein, 290 aa, Homo sapiens
MYHGMNPSNGDGFLEQQQQQQQPQSPQRLLAVILWFQLALCFGPAQLTGGFDDLQVCADPGIPENGFRTPSGGVFFEGSVARFHCQDGFKLKGATKRLCLKHFNGTLGWIPSDNSICVQEDCRIPQIEDAEIHNKTYRHGEKLIITCHEGFKIRYPDLHNMVSLCRDDGTWNNLPICQGCLRPLASSNGYVNISELQTSFPVGTVISYRCFPGFKLDGSAYLECLQNLIWSSSPPRCLALEGGRPEHLFPVLYFPHIRLAAAVLYFCPVLKSSPTPAPTCSSTSTTTSLF

SEQ ID NO: 187 - DNA, 29 bp, Artificial Sequence
  oligonucleotide
  misc_feature (2): biotinylated phosphoaramidite residue
antgacttcagttgagggcaagtctctgg

SEQ ID NO: 188 - DNA, 29 bp, Artificial Sequence
  oligonucleotide
  misc_feature (2): biotinylated phosphoaramidite residue
tncagaaagactgcagggattcgggacaa

SEQ ID NO: 189 - DNA, 29 bp, Artificial Sequence
  oligonucleotide
  misc_feature (2): biotinylated phosphoaramidite residue
antcatcactacacgtcttctcccctaca

SEQ ID NO: 190 - DNA, 29 bp, Artificial Sequence
  oligonucleotide
  misc_feature (2): biotinylated phosphoaramidite residue
gnctgagtatgttgtggaatgggctgcaa

SEQ ID NO: 191 - DNA, 29 bp, Artificial Sequence
  oligonucleotide
  misc_feature (2): biotinylated phosphoaramidite residue
tngtgactgtatacctgcaacctcaatgc

SEQ ID NO: 192 - DNA, 29 bp, Artificial Sequence
  oligonucleotide
  misc_feature (2): biotinylated phosphoaramidite residue
tngccttgacacaggtggcagaagaaact

SEQ ID NO: 193 - DNA, 29 bp, Artificial Sequence
  oligonucleotide
  misc_feature (2): biotinylated phosphoaramidite residue
cngactggtagtgacaccaagagaatgga

SEQ ID NO: 194 - DNA, 29 bp, Artificial Sequence
  oligonucleotide
  misc_feature (2): biotinylated phosphoaramidite residue
anagcacagcttagttttcagtgactcca

SEQ ID NO: 195 - DNA, 20 bp, Artificial Sequence
  oligonucleotide
gcatatactctgttgcccgc

SEQ ID NO: 196 - DNA, 18 bp, Artificial Sequence
  oligonucleotide
ctgccactatccccaggg

SEQ ID NO: 197 - DNA, 29 bp, Artificial Sequence
  oligonucleotide
  misc_feature (2): biotinylated phosphoaramidite residue
antggtgtgccactcccaacaatctttcc

SEQ ID NO: 198 - DNA, 2505 bp, Homo sapiens
ccagctccccactgccctgagggcgggccggcctgcggcggagggaaaaaggaagaggagaaggaaattgtcccgaatccctgcaggtcagtacctggaagattccataaagtcggggtgcttgagggcgtagggccgagaccgtcgcgggtactgaggcgcctccgtcgtctctcccactcgccgcccgctttccaagacatatgtcccgcttgcagcccatttcgatgctgcgaaacggtgagctgcggggtgtttggggaagagctcagagactgggaaatgggaatctgctgggagcctagggccgcaatccggaaagggagctgtggcctgggtgttggcccctagtccaccaggacagtgccggaggggaatggctggatatgggggcgggggtggtgagatgcaacgcgatatgtcagcagaaccccaagagaggtaataggggtgggaaacctctgacaaccaggcctccgaattagaaaagagttttgtgttctggggactagtccgtccaccaagcgctcagtggcggcagtttcccgtctttctgcctgtggctgtgtcttactgaccatggctctgtgtctagtgggtccaagcctctcccgggtggccagtctttctgtaggttgcggcacaacgccaggcaaaagaagaggaaggaatttaatcctaatcggtggaggtcgatttgagggtctgctgtagcaggtggctccgcttgaagcgagggaggaagtttcctccgatcagtagagattggaaagattgttgggagtggcacaccactagggaaaagaagaaggggcgaactgcttgtcttgaggaggtcaacccccagaatcagctcttgtggccttgaagtggctgaagacgatcaccctccacaggcttgagcccagtcccacagccttcctcccccagcctgagtgactactctattccttggtccctgctattgtcggggacgattgcatgggctacgccaggaaagtaggctgggtgaccgcaggcctggtgattggggctggcgcctgctattgcatttatagactgactaggggaagaaaacagaacaaggaaaaaatggctgagggtggatctggggatgtggatgatgctggggactgttctggggccaggtataatgactggtctgatgatgatgatgacagcaatgagagcaagagtatagtatggtacccaccttgggctcggattgggactgaagctggaaccagagctagggccagggcaagggccagggctacccgggcacgtcgggctgtccagaaacgggcttcccccaattcagatgataccgttttgtcccctcaagagctacaaaaggttctttgcttggttgagatgtctgaaaagccttatattcttgaagcagctttaattgctctgggtaacaatgctgcttatgcatttaacagagatattattcgtgatctgggtggtctcccaattgtcgcaaagattctcaatactcgggatcccatagttaaggaaaaggctttaattgtcctgaataacttgagtgtgaatgctgaaaatcagcgcaggcttaaagtatacatgaatcaagtgtgtgatgacacaatcacttctcgcttgaactcatctgtgcagcttgctggactgagattgcttacaaatatgactgttactaatgagtatcagcacatgcttgctaattccatttctgacttttttcgtttattttcagcgggaaatgaagaaaccaaacttcaggttctgaaactccttttgaatttggctgaaaatccagccatgactagggaactgctcagggcccaagtaccatcttcactgggctccctctttaataagaaggagaacaaagaagttattcttaaacttctggtcatatttgagaacataaatgataatttcaaatgggaagaaaatgaacctactcagaatcaattcggtgaaggttcactttttttctttttaaaagaatttcaagtgtgtgctgataaggttctgggaatagaaagtcaccatgattttttggtgaaagtaaaagttggaaaattcatggccaaacttgctgaacatatgttcccaaagagccaggaataacaccttgattttgtaatttagaagcaacacacattgtaaactattcattttctccaccttgtttatatggtaaaggaatcctttcagctgccagttttgaataatgaatatcatattgtatcatcaatgctgatatttaactgagttggtctttaggtttaagatggataaatgaatatcactacttgttctgaaaacatgtttgttgctttttatctcgctgcctagattgaaatattttgctatttcttctgcataagtgacagtgaaccaattcatcatgagtaagctcccttctgtcattttcattgatttaatttgtgtatcatcaataaaattgtatgttaatgctggaaagaaaaaaaaaaaaaaaaaaaaaaaaaa

SEQ ID NO: 199 - DNA, 29 bp, Artificial Sequence
  oligonucleotide
  misc_feature (2): biotinylated phosphoaramidite residue
gntgaaacctgaaggatggagagaaatta

SEQ ID NO: 200 - DNA, 29 bp, Artificial Sequence
  oligonucleotide
  misc_feature (2): biotinylated phosphoaramidite residue
gngagaaatacatcagagcaggctgccat

SEQ ID NO: 201 - DNA, 29 bp, Artificial Sequence
  oligonucleotide
  misc_feature (2): biotinylated phosphoaramidite residue
tngtattgcatataagctacaactttacc

SEQ ID NO: 202 - DNA, 29 bp, Artificial Sequence
  oligonucleotide
  misc_feature (2): biotinylated phosphoaramidite residue
antaaagtacctatgcagttttaagacca

SEQ ID NO: 203 - DNA, 29 bp, Artificial Sequence
  oligonucleotide
  misc_feature (2): biotinylated phosphoaramidite residue
gngcaaagaacagaggattcttgagaaag

SEQ ID NO: 204 - DNA, 29 bp, Artificial Sequence
  oligonucleotide
  misc_feature (2): biotinylated phosphoaramidite residue
anactggcctaggtttcagggttgtatca

SEQ ID NO: 205 - DNA, 29 bp, Artificial Sequence
  oligonucleotide
  misc_feature (2): biotinylated phosphoaramidite residue
cntgccctaactagacaattacgaatccc

SEQ ID NO: 206 - DNA, 29 bp, Artificial Sequence
  oligonucleotide
  misc_feature (2): biotinylated phosphoaramidite residue
angaaagagccttctgtgctgttgataaa

SEQ ID NO: 207 - DNA, 29 bp, Artificial Sequence
  oligonucleotide
  misc_feature (2): biotinylated phosphoaramidite residue
cnctgccagccccacactctcatcacaaa

SEQ ID NO: 208 - DNA, 19 bp, Artificial Sequence
  oligonucleotide
tcctcaccctcttcccttg

SEQ ID NO: 209 - DNA, 29 bp, Artificial Sequence
  oligonucleotide
  misc_feature (2): biotinylated phosphoaramidite residue
cnaattgttcaggttgtagagatgtcagc

SEQ ID NO: 210 - DNA, 29 bp, Artificial Sequence
  oligonucleotide
  misc_feature (2): biotinylated phosphoaramidite residue
tnagaaggaaatggaaacacacgggaaat

SEQ ID NO: 211 - DNA, 29 bp, Artificial Sequence
  oligonucleotide
  misc_feature (2): biotinylated phosphoaramidite residue
tnagcatgaccagtggtggagcaacgaag

SEQ ID NO: 212 - DNA, 20 bp, Artificial Sequence
  oligonucleotide
ggtatgggaagctagagggc

SEQ ID NO: 213 - DNA, 18 bp, Artificial Sequence
  oligonucleotide
gtctgggacgatgttggc

SEQ ID NO: 214 - DNA, 29 bp, Artificial Sequence
  oligonucleotide
  misc_feature (2): biotinylated phosphoaramidite residue
cngagagctattgtccttgagtaggctga

SEQ ID NO: 215 - DNA, 29 bp, Artificial Sequence
  oligonucleotide
  misc_feature (2): biotinylated phosphoaramidite residue
gnatcttgtgtcagccccaaaggtttcag

SEQ ID NO: 216 - DNA, 29 bp, Artificial Sequence
  oligonucleotide
  misc_feature (2): biotinylated phosphoaramidite residue
antacaacatgggatgttcaggactaatc

SEQ ID NO: 217 - DNA, 29 bp, Artificial Sequence
  oligonucleotide
  misc_feature (2): biotinylated phosphoaramidite residue
cngcagcagcagctgcccgtttcatcatg

SEQ ID NO: 218 - DNA, 29 bp, Artificial Sequence
  oligonucleotide
  misc_feature (2): biotinylated phosphoaramidite residue
cngggctaacagcccgtagaagacaatga

SEQ ID NO: 219 - DNA, 29 bp, Artificial Sequence
  oligonucleotide
  misc_feature (2): biotinylated phosphoaramidite residue
cnctaggagagatgctttcacagggtaaa

SEQ ID NO: 220 - DNA, 29 bp, Artificial Sequence
  oligonucleotide
  misc_feature (2): biotinylated phosphoaramidite residue
cngtgggaagcagaacaacagaaggaact

SEQ ID NO: 221 - DNA, 29 bp, Artificial Sequence
  oligonucleotide
  misc_feature (2): biotinylated phosphoaramidite residue
gntcagcagcacagaggagacaaagtaca

SEQ ID NO: 222 - DNA, 29 bp, Artificial Sequence
  oligonucleotide
  misc_feature (2): biotinylated phosphoaramidite residue
angttgaaggtcgatgttttctcttgctg

SEQ ID NO: 223 - DNA, 29 bp, Artificial Sequence
  oligonucleotide
  misc_feature (2): biotinylated phosphoaramidite residue
gnctgatgatgccaaccaagatagttcta

SEQ ID NO: 224 - DNA, 29 bp, Artificial Sequence
  oligonucleotide
  misc_feature (2): biotinylated phosphoaramidite residue
gngaggacagttcttttggaggttggagg

SEQ ID NO: 225 - DNA, 29 bp, Artificial Sequence
  oligonucleotide
  misc_feature (2): biotinylated phosphoaramidite residue
anttaagacgaatgtgtgggtttcagacc

SEQ ID NO: 226 - DNA, 29 bp, Artificial Sequence
  oligonucleotide
  misc_feature (2): biotinylated phosphoaramidite residue
tntcaacatcccaagtagacagcagtcct

SEQ ID NO: 227 - DNA, 29 bp, Artificial Sequence
  oligonucleotide
  misc_feature (2): biotinylated phosphoaramidite residue
tngacccacagagagcagggacttcacaa

SEQ ID NO: 228 - DNA, 29 bp, Artificial Sequence
  oligonucleotide
  misc_feature (2): biotinylated phosphoaramidite residue
tngtttccttccagagggaatgcagtatg

SEQ ID NO: 229 - DNA, 29 bp, Artificial Sequence
  oligonucleotide
  misc_feature (2): biotinylated phosphoaramidite residue
gncggtaccagtagcaatgagcacgaagg

SEQ ID NO: 230 - DNA, 29 bp, Artificial Sequence
  oligonucleotide
  misc_feature (2): biotinylated phosphoaramidite residue
tncgcgagctcctaattcctgctcctcag

SEQ ID NO: 231 - DNA, 29 bp, Artificial Sequence
  oligonucleotide
  misc_feature (2): biotinylated phosphoaramidite residue
gnaaatctatgtcatcttgtcgggaccaa

SEQ ID NO: 232 - DNA, 29 bp, Artificial Sequence
  oligonucleotide
  misc_feature (2): biotinylated phosphoaramidite residue
tnaggaagatgggaggtaacccaagggaa

SEQ ID NO: 233 - DNA, 29 bp, Artificial Sequence
  oligonucleotide
  misc_feature (2): biotinylated phosphoaramidite residue
tncagatccatcaatgagggtccacccag

SEQ ID NO: 234 - DNA, 29 bp, Artificial Sequence
  oligonucleotide
  misc_feature (2): biotinylated phosphoaramidite residue
gncctgtgtgcccagaacaatcatgctcc

SEQ ID NO: 235 - DNA, 18 bp, Artificial Sequence
  oligonucleotide
gtttctggaatgcgggtg

SEQ ID NO: 236 - DNA, 19 bp, Artificial Sequence
  oligonucleotide
ccgtgataccgaaatgtcc

SEQ ID NO: 237 - DNA, 29 bp, Artificial Sequence
  oligonucleotide
  misc_feature (2): biotinylated phosphoaramidite residue
gnaacaatcaccttccacatggcaccaac

SEQ ID NO: 238 - DNA, 29 bp, Artificial Sequence
  oligonucleotide
  misc_feature (2): biotinylated phosphoaramidite residue
gngttgaggcagagctcagtggtgtccac

SEQ ID NO: 239 - DNA, 29 bp, Artificial Sequence
  oligonucleotide
  misc_feature (2): biotinylated phosphoaramidite residue
ancgtgtgtacgatctgtagggctgtctg

SEQ ID NO: 240 - DNA, 29 bp, Artificial Sequence
  oligonucleotide
  misc_feature (2): biotinylated phosphoaramidite residue
gnagcacgcggaaccaacacgttctaata

SEQ ID NO: 241 - DNA, 29 bp, Artificial Sequence
  oligonucleotide
  misc_feature (2): biotinylated phosphoaramidite residue
anatcagggagctgaggcttagagagaga

SEQ ID NO: 242 - DNA, 29 bp, Artificial Sequence
  oligonucleotide
  misc_feature (2): biotinylated phosphoaramidite residue
gngaaaggagagaaggcccaagagagagg

SEQ ID NO: 243 - DNA, 29 bp, Artificial Sequence
  oligonucleotide
  misc_feature (2): biotinylated phosphoaramidite residue
gntgccactgacgaaagcttgaaataacc

SEQ ID NO: 244 - DNA, 20 bp, Artificial Sequence
  oligonucleotide
ggctctacatctcatcaccc

SEQ ID NO: 245 - DNA, 29 bp, Artificial Sequence
  oligonucleotide
  misc_feature (2): biotinylated phosphoaramidite residue
cnaagttctattgggagatggagtttgtg

SEQ ID NO: 246 - DNA, 29 bp, Artificial Sequence
  oligonucleotide
  misc_feature (2): biotinylated phosphoaramidite residue
cnatccatggtacatggtcagaagctcat

SEQ ID NO: 247 - DNA, 29 bp, Artificial Sequence
  oligonucleotide
  misc_feature (2): biotinylated phosphoaramidite residue
tngagcaggtcaggatacactggaaaaga

SEQ ID NO: 248 - DNA, 29 bp, Artificial Sequence
  oligonucleotide
  misc_feature (2): biotinylated phosphoaramidite residue
cnactgcctttgttgctttccagtagtga

SEQ ID NO: 249 - DNA, 29 bp, Artificial Sequence
  oligonucleotide
  misc_feature (2): biotinylated phosphoaramidite residue
tnaatatccacatccccaaatcctacacg

SEQ ID NO: 250 - DNA, 29 bp, Artificial Sequence
  oligonucleotide
  misc_feature (2): biotinylated phosphoaramidite residue
cncttgcagcgggaaggcagagaagtttc

SEQ ID NO: 251 - DNA, 29 bp, Artificial Sequence
  oligonucleotide
  misc_feature (2): biotinylated phosphoaramidite residue
cntgagccacaatagacagaattcctacc

SEQ ID NO: 252 - DNA, 29 bp, Artificial Sequence
  oligonucleotide
  misc_feature (2): biotinylated phosphoaramidite residue
cngtcagggcgcagctgtattggtcacaa

SEQ ID NO: 253 - DNA, 19 bp, Artificial Sequence
  oligonucleotide
acccacacagaagtgagcc

SEQ ID NO: 254 - DNA, 29 bp, Artificial Sequence
  oligonucleotide
  misc_feature (2): biotinylated phosphoaramidite residue
tnaccagtgtgcgaaggtagagacggcat

SEQ ID NO: 255 - DNA, 29 bp, Artificial Sequence
  oligonucleotide
  misc_feature (2): biotinylated phosphoaramidite residue
tntagcccgatgaggctgtatgagtacag

SEQ ID NO: 256 - DNA, 29 bp, Artificial Sequence
  oligonucleotide
  misc_feature (2): biotinylated phosphoaramidite residue
tntcactgccaaacggagaagaaacgcaa

SEQ ID NO: 257 - DNA, 29 bp, Artificial Sequence
  oligonucleotide
  misc_feature (2): biotinylated phosphoaramidite residue
gngaaggaccaagacaatccctgaagtaa

SEQ ID NO: 258 - DNA, 20 bp, Artificial Sequence
  oligonucleotide
ttggagcactgaggaacaag

SEQ ID NO: 259 - DNA, 29 bp, Artificial Sequence
  oligonucleotide
  misc_feature (2): biotinylated phosphoaramidite residue
gncgtctgcaggagatcaaaaacactgtc

SEQ ID NO: 260 - DNA, 29 bp, Artificial Sequence
  oligonucleotide
  misc_feature (2): biotinylated phosphoaramidite residue
angcagcagggattgagaagggaacatca

SEQ ID NO: 261 - DNA, 29 bp, Artificial Sequence
  oligonucleotide
  misc_feature (2): biotinylated phosphoaramidite residue
tnagtttcaccagtctgagcacaagtttg

SEQ ID NO: 262 - DNA, 29 bp, Artificial Sequence
  oligonucleotide
  misc_feature (2): biotinylated phosphoaramidite residue
anggatcacttctgcctctgcttcctgga

SEQ ID NO: 263 - DNA, 29 bp, Artificial Sequence
  oligonucleotide
  misc_feature (2): biotinylated phosphoaramidite residue
antggacacttccatacacactaggtgaa

SEQ ID NO: 264 - DNA, 29 bp, Artificial Sequence
  oligonucleotide
  misc_feature (2): biotinylated phosphoaramidite residue
gncatggaaggagactgggataaggcaga

SEQ ID NO: 265 - DNA, 29 bp, Artificial Sequence
  oligonucleotide
  misc_feature (2): biotinylated phosphoaramidite residue
tnccaggaacacagaaaaaaacttgagaa

SEQ ID NO: 266 - DNA, 29 bp, Artificial Sequence
  oligonucleotide
  misc_feature (2): biotinylated phosphoaramidite residue
gngctgggagtactgctagagggtgtgga

SEQ ID NO: 267 - DNA, 29 bp, Artificial Sequence
  oligonucleotide
  misc_feature (2): biotinylated phosphoaramidite residue
cnctctttggctgtacacgaacttgctcc

SEQ ID NO: 268 - DNA, 29 bp, Artificial Sequence
  oligonucleotide
  misc_feature (2): biotinylated phosphoaramidite residue
gngggtggcacagcagagaaagactccat

SEQ ID NO: 269 - DNA, 29 bp, Artificial Sequence
  oligonucleotide
  misc_feature (2): biotinylated phosphoaramidite residue
tngcatcttcaccgccagcatcagttttg

SEQ ID NO: 270 - DNA, 29 bp, Artificial Sequence
  oligonucleotide
  misc_feature (2): biotinylated phosphoaramidite residue
cnaactctgtaaagccaagtccagtcacc

SEQ ID NO: 271 - DNA, 29 bp, Artificial Sequence
  oligonucleotide
  misc_feature (2): biotinylated phosphoaramidite residue
tnctgaggttgcctccaatttctccatct

SEQ ID NO: 272 - DNA, 29 bp, Artificial Sequence
  oligonucleotide
  misc_feature (2): biotinylated phosphoaramidite residue
gntgacaaaccaaaaataacaaagacccc

SEQ ID NO: 273 - DNA, 29 bp, Artificial Sequence
  oligonucleotide
  misc_feature (2): biotinylated phosphoaramidite residue
gntacatctttcatccacagagggcatcc

SEQ ID NO: 274 - protein, 51 aa, Homo sapiens
MVLFFFFFSLAVPCSLPSLDVCTNYSLELFSLALQLLPPTSSPAPPIHSFA

SEQ ID NO: 275 - protein, 82 aa, Homo sapiens
  UNSURE (48)
MNVYTHFRGSHQGQVQGSGPSGWCLQGNFGPSLFSDWRSPWPASFHTXLLAGTGLAPTFPASSVVASLPEPGSSSGPTSKCH

SEQ ID NO: 276 - protein, 130 aa, Homo sapiens
MDDMLSTRSSTLTEDGAKSSEAIKESSKFPFGISPAQSHRNIKILEDEPHSKDETPLCTLLDWQDSLAKRCVCVSNTIRSLSFVPGNDFEMSKHPGLLLILGKLILLHHKHPERKQAPLTYEKEEEQDQG

SEQ ID NO: 277 - protein, 111 aa, Homo sapiens [sequence omitted]

SEQ ID NO: 278 - protein, 104 aa, Homo sapiens [sequence omitted]

SEQ ID NO: 279 - protein, 147 aa, Homo sapiens
MASPSGLCVLVRLPKLICGGKTLPRTLLDILADGTILKVGVGCSEDASKLLQDYGLVVRGCLDLRYLAMRQRNNLLCNGLSLKSLAETVLNFPLDKSLLLRCSNWDAETLTEDQVIYAARDAQISVALFLHLLGYPFSRNSPGEKKR

SEQ ID NO: 280 - protein, 176 aa, Homo sapiens
MTDCLVIKHFLRKIIMVHPKVRFHFSVKVNGILSTEIFGVENEPTLNLGNGIALLVDSQHYVSRPNFGTIESHCSRIHPVLGHPVMLFIPEDVAGMDLLGELILTPAAALCPSPKVSSNQLNRISSVSIFLYGPLGLPLILSTWEQPMTTFFKDTSSLVDWKIPFVYDTQFGSQFG

SEQ ID NO: 281 - protein, 89 aa, Homo sapiens
MGSLSTANVEFCLDVFKELNSNNIGDNIFFSSLSLLYALSMVLLGARGETAEQLEKVLHFSHTVDSLKPGFKDSPKCSQAGRIHSEFGV

SEQ ID NO: 282 - protein, 115 aa, Homo sapiens
MVTGMLISSTRGSSDGRNCSAILVPVSPVGRQPLYLTSRPGDWSQGYCTTGQFPAIVRKETPELNGRDIPAVFNITPMPFVRLPCTEITWRASCRLYLRTLVKYLLSFLAARMQK

SEQ ID NO: 283 - protein, 189 aa, Homo sapiens
MVHCPHELLQMPLSLFSQRSWVTQCLDTWKTCTLITQRHLASDHLPSEFLLVQLGYHPLTHQAAPHLPLLLLWQVFPAYQEQGFSCKGQLLLGLLVSTDNIFLPILGQAPQTHPLLPHQRWANQKESVPVKIERYLPQLEQRDWPEFGKEGLLHKPRRGPVLSLPLDTVESGHLVSMLCQKAYQVGRNL

(19) United States
(12) Patent Application Publication
(10) Pub. No.: US 2001/0039335 A1
(43) Pub. Date: Nov. 8, 2001

(54) Secreted proteins and polynucleotides encoding them

(76) Inventors: Kenneth Jacobs, Newton, MA (US); John M. McCoy, Reading, MA (US); Edward R. LaVallie, Harvard, MA (US); Lisa A. Collins-Racie, Acton, MA (US); Cheryl Evans, Germantown, MD (US); David Merberg, Acton, MA (US); Maurice Treacy, Co. Dublin (IE); Michael J. Agostino, Andover, MA (US); Robert J. Steininger II, Cambridge, MA (US); Vikki Spaulding, Lowell, MA (US); Gordon G. Wong, Brookline, MA (US); Hilary Clark, So. San Francisco, CA (US); Kim Fechtel, Arlington, MA (US)

Correspondence Address: Alice O. Carroll, Esq., HAMILTON, BROOK, SMITH & REYNOLDS, P.C., Two Militia Drive, Lexington, MA, 02421-4799, US

(21) Appl. No.: 09/729,674

(22) Filed: Dec. 4, 2000

Related U.S. Application Data
(63) Continuation of application No. 09/539,330, filed on Mar. 30, 2000.
Application No. 09/539,330 is a continuation-in-part of application No. 09/197,886, filed on Nov. 23, 1998, now abandoned.
(60) Provisional application No. 60/126,425, filed on Apr. 10, 1997.
(60) Provisional application No. 60/067,454, filed on Dec. 4, 1997.
(60) Provisional application No. 60/068,379, filed on Dec. 20, 1997.
(60) Provisional application No. 60/070,346, filed on Jan. 2, 1998.
(60) Provisional application No. 60/070,643, filed on Jan. 7, 1998.
(60) Provisional application No. 60/070,755, filed on Jan. 8, 1998.
(60) Provisional application No. 60/071,304, filed on Jan. 13, 1998.
(60) Provisional application No. 60/072,134, filed on Jan. 22, 1998.
(60) Provisional application No. 60/073,095, filed on Jan. 30, 1998.
(60) Provisional application No. 60/075,038, filed on Feb. 18, 1998.

(51) Int. Cl. C07H 21/04; C12P 21/02; C12N 5/06; C07K 14/52

(52) U.S. Cl. 536/23.5; 530/351; 435/69.5; 435/325

(57) ABSTRACT

Novel polynucleotides and the proteins encoded thereby are disclosed.

This application is a continuation-in-part of the following applications:

(1) Ser. No. 09/197,886 (GI 6055A), filed Nov. 23,1998; which is a continuation-in-part of provisional application Ser. No.60/126,425 (GI 6055), filed Nov. 26,1997, now abandoned;

(2) Ser. No.09/203,106 (GI 6056A), filed Nov. 30,1998; which is a continuation-in-part of provisional application Ser. No. 60/067,454 (GI 6056), filed Dec. 4,1997, now abandoned;

(3) Ser. No.09/212,843 (GI 6057A), filed Dec. 16,1998; which is a continuation-in-part of provisional application Ser. No. 60/068,379 (GI 6057), filed Dec. 20, 1997, now abandoned;

(4) Ser. No.09/227,653 (GI 6058A), filed Dec. 30,1998; which is a continuation-in-part of provisional application Ser. No. 60/070,346 (GI 6058), filed Jan. 2, 1998, now abandoned;

(5) Ser. No. 09/225,049 (GI 6059A), filed Jan. 4,1999; which is a continuation-in-part of provisional application Ser. No.60/070,643 (GI 6059), filed Jan. 7,1998, now abandoned;

(6) Ser. No. 09/225,585 (GI 6060A), filed Jan. 6,1999; which is a continuation-in-part of provisional application Ser. No.60/070,755 (GI 6060), filed Jan. 8,1998, now abandoned;

(7) Ser. No. 09 /227,462 (GI 6061A), filed Jan. 8, 1999; which is a continuation-in-part of provisional application Ser. No. 60/071,304 (GI 6061), filed Jan. 13, 1998, now abandoned;

(8) Ser. No.09/235,609 (GI 6062A), filed Jan. 20,1999; which is a continuation-in-part of provisional application Ser. No. 60/072,134 (GI 6062), filed Jan. 22, 1998, now abandoned;

(9) Ser. No.09/237,847 (GI 6063A), filed Jan. 27,1999; which is a continuation-in-part of provisional application Ser. No. 60/073,095 (GI 6063), filed Jan. 30, 1998, now abandoned;

(10) Ser. No. 09/251,600 (GI 6064A), filed Feb. 17,1999; which is a continuation-in-part of provisional application Ser. No.60/075,038 (GI 6064), filed Feb. 18, 1998, now abandoned;

all of which are incorporated by reference herein.

### FIELD OF THE INVENTION

The present invention provides novel polynucleotides and proteins encoded by such polynucleotides, along with therapeutic, diagnostic and research utilities for these polynucleotides and proteins.

### BACKGROUND OF THE INVENTION

Technology aimed at the discovery of protein factors (including e.g., cytokines, such as lymphokines, interferons, CSFs and interleukins) has matured rapidly over the past decade. The now routine hybridization cloning and expression cloning techniques clone novel polynucleotides “directly” in the sense that they rely on information directly related to the discovered protein (i.e., partial DNA/amino acid sequence of the protein in the case of hybridization cloning; activity of the protein in the case of expression cloning). More recent “indirect” cloning techniques such as signal sequence cloning, which isolates DNA sequences based on the presence of a now well-recognized secretory leader sequence motif, as well as various PCR-based or low stringency hybridization cloning techniques, have advanced the state of the art by making available large numbers of DNA/ amino acid sequences for proteins that are known to have biological activity by virtue of their secreted nature in the case of leader sequence cloning, or by virtue of the cell or tissue source in the case of PCR-based techniques. It is to these proteins and the polynucleotides encoding them that the present invention is directed.

### SUMMARY OF THE INVENTION

In one embodiment, the present invention provides a composition comprising an isolated polynucleotide selected from the group consisting of:

(a) a polynucleotide comprising the nucleotide sequence of SEQ ID NO:1;

(b) a polynucleotide comprising the nucleotide sequence of SEQ ID NO:1 from nucleotide 63 to nucleotide 1265;

(c) a polynucleotide comprising the nucleotide sequence of SEQ ID NO:1 from nucleotide 132 to nucleotide 1265;

(d) a polynucleotide comprising the nucleotide sequence of the full-length protein coding sequence of clone bd306_{—}7 deposited with the ATCC under accession number 98599;

(e) a polynucleotide encoding the full-length protein encoded by the cDNA insert of clone bd306_{—}7 deposited with the ATCC under accession number 98599;

(f) a polynucleotide comprising the nucleotide sequence of a mature protein coding sequence of clone bd306_{—}7 deposited with the ATCC under accession number 98599;

(g) a polynucleotide encoding a mature protein encoded by the cDNA insert of clone bd306_{—}7 deposited with the ATCC under accession number 98599;

(h) a polynucleotide encoding a protein comprising the arnino acid sequence of SEQ ID NO:2;

(i) a polynucleotide encoding a protein comprising a fragment of the amino acid sequence of SEQ ID NO:2 having biological activity, the fragment comprising eight consecutive amino acids of SEQ ID NO:2;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:1 from nucleotide 63 to nucleotide 1265; the nucleotide sequence of SEQ ID NO:1 from nucleotide 132 to nucleotide 1265; the nucleotide sequence of the full-length protein coding sequence of clone bd306_{—}7 deposited with the ATCC under accession number 98599; or the nucleotide sequence of a mature protein coding sequence of clone bd306_{—}7 deposited with the ATCC under accession number 98599. In other preferred embodiments, the polynucleotide encodes the full-length or a mature protein encoded by the cDNA insert of clone bd306_{—}7 deposited with the ATCC under accession number 98599. In yet other preferred embodiments, the present invention provides a polynucleotide encoding a protein comprising the amino acid sequence of SEQ ID NO:2 from amino acid 148 to amino acid 189. In further preferred embodiments, the present invention provides a polynucleotide encoding a protein comprising a fragment of the amino acid sequence of SEQ ID NO:2 having biological activity, the fragment preferably comprising eight (more preferably twenty, most preferably thirty) consecutive amino acids of SEQ ID NO:2, or a polynucleotide encoding a protein comprising a fragment of the amino acid sequence of SEQ ID NO:2 having biological activity, the fragment comprising the amino acid sequence from amino acid 195 to amino acid 204 of SEQ ID NO:2.

Other embodiments provide the gene corresponding to the cDNA sequence of SEQ ID NO:1.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:1, but excluding the poly(A) tail at the 3′ end of SEQ ID NO:1; and

(ab) the nucleotide sequence of the cDNA insert of clone bd306_{—}7 deposited with the ATCC under accession number 98599; and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:1, but excluding the poly(A) tail at the 3′ end of SEQ ID NO:1; and

(bb) the nucleotide sequence of the cDNA insert of clone bd306_{—}7 deposited with the ATCC under accession number 98599; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:1, and extending contiguously from a nucleotide sequence corresponding to the 5′ end of SEQ ID NO:1 to a nucleotide sequence corresponding to the 3′ end of SEQ ID NO:1, but excluding the poly(A) tail at the 3′ end of SEQ ID NO:1. Also preferably the polynucleotide isolated according to the above process comprises a nucleotide sequence corresponding to the cDNA sequence of SEQ ID NO:1 from nucleotide 63 to nucleotide 1265, and extending contiguously from a nucleotide sequence corresponding to the 5′ end of said sequence of SEQ ID NO:1 from nucleotide 63 to nucleotide 1265, to a nucleotide sequence corresponding to the 3′ end of said sequence of SEQ ID NO:1 from nucleotide 63 to nucleotide 1265. Also preferably the polynucleotide isolated according to the above process comprises a nucleotide sequence corresponding to the cDNA sequence of SEQ ID NO:1 from nucleotide 132 to nucleotide 1265, and extending contiguously from a nucleotide sequence corresponding to the 5′ end of said sequence of SEQ ID NO:1 from nucleotide 132 to nucleotide 1265, to a nucleotide sequence corresponding to the 3′ end of said sequence of SEQ ID NO:1 from nucleotide 132 to nucleotide 1265.

In other embodiments, the present invention provides a composition comprising a protein, wherein said protein comprises an amino acid sequence selected from the group consisting of:

(a) the amino acid sequence of SEQ ID NO:2;

(b) the amino acid sequence of SEQ ID NO:2 from amino acid 148 to amino acid 189;

(c) fragments of the amino acid sequence of SEQ ID NO:2 comprising eight consecutive amino acids of SEQ ID NO:2; and

(d) the amino acid sequence encoded by the cDNA insert of clone bd306_{—}7 deposited with the ATCC under accession number 98599;

the protein being substantially free from other mammalian proteins. Preferably such protein comprises the amino acid sequence of SEQ ID NO:2 or the amino acid sequence of SEQ ID NO:2 from amino acid 148 to amino acid 189. In further preferred embodiments, the present invention provides a protein comprising a fragment of the amino acid sequence of SEQ ID NO:2 having biological activity, the fragment preferably comprising eight (more preferably twenty, most preferably thirty) consecutive amino acids of SEQ ID NO:2, or a protein comprising a fragment of the amino acid sequence of SEQ ID NO:2 having biological activity, the fragment comprising the amino acid sequence from amino acid 195 to amino acid 204 of SEQ ID NO:2.

In one embodiment, the present invention provides a composition comprising an isolated polynucleotide selected from the group consisting of:

(a) a polynucleotide comprising the nucleotide sequence of SEQ ID NO:3;

(b) a polynucleotide comprising the nucleotide sequence of SEQ ID NO:3 from nucleotide 719 to nucleotide 1855;

(c) a polynucleotide comprising the nucleotide sequence of SEQ ID NO:3 from nucleotide 779 to nucleotide 1855;

(d) a polynucleotide comprising the nucleotide sequence of the full-length protein coding sequence of clone fj283_{—}11 deposited with the ATCC under accession number 98599;

(e) a polynucleotide encoding the full-length protein encoded by the cDNA insert of clone fj283_{—}11 deposited with the ATCC under accession number 98599;

(f) a polynucleotide comprising the nucleotide sequence of a mature protein coding sequence of clone fj28311 deposited with the ATCC under accession number 98599;

(g) a polynucleotide encoding a mature protein encoded by the cDNA insert of clone fj283_{—}11 deposited with the ATCC under accession number 98599;

(h) a polynucleotide encoding a protein comprising the amino acid sequence of SEQ ID NO:4;

(i) a polynucleotide encoding a protein comprising a fragment of the amino acid sequence of SEQ ID NO:4 having biological activity, the fragment comprising eight consecutive amino acids of SEQ ID NO:4;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:3 from nucleotide 719 to nucleotide 1855; the nucleotide sequence of SEQ ID NO:3 from nucleotide 779 to nucleotide 1855; the nucleotide sequence of the full-length protein coding sequence of clone fj283_{—}11 deposited with the ATCC under accession number 98599; or the nucleotide sequence of a mature protein coding sequence of clone fj283_{—}11 deposited with the ATCC under accession number 98599. In other preferred embodiments, the polynucleotide encodes the full-length or a mature protein encoded by the cDNA insert of clone fj283_{—}11 deposited with the ATCC under accession number 98599. In yet other preferred embodiments, the present invention provides a polynucleotide encoding a protein comprising the amino acid sequence of SEQ ID NO:4 from amino acid 1 to amino acid 27. In further preferred embodiments, the present invention provides a polynucleotide encoding a protein comprising a fragment of the amino acid sequence of SEQ ID NO:4 having biological activity, the fragment preferably comprising eight (more preferably twenty, most preferably thirty) consecutive amino acids of SEQ ID NO:4, or a polynucleotide encoding a protein comprising a fragment of the amino acid sequence of SEQ ID NO:4 having biological activity, the fragment comprising the amino acid sequence from amino acid 184 to amino acid 193 of SEQ ID NO:4.

Other embodiments provide the gene corresponding to the cDNA sequence of SEQ ID NO:3.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:3, but excluding the poly(A) tail at the 3′ end of SEQ ID NO:3; and

(ab) the nucleotide sequence of the cDNA insert of clone f283_{—}11 deposited with the ATCC under accession number 98599; and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:3, but excluding the poly(A) tail at the 3′ end of SEQ ID NO:3; and

(bb) the nucleotide sequence of the cDNA insert of clone fj283_{—}11 deposited with the ATCC under accession number 98599; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:3, and extending contiguously from a nucleotide sequence corresponding to the 5′ end of SEQ ID NO:3 to a nucleotide sequence corresponding to the 3′ end of SEQ ID NO:3 , but excluding the poly(A) tail at the 3′ end of SEQ ID NO:3. Also preferably the polynucleotide isolated according to the above process comprises a nucleotide sequence corresponding to the cDNA sequence of SEQ ID NO:3 from nucleotide 719 to nucleotide 1855, and extending contiguously from a nucleotide sequence corresponding to the 5′ end of said sequence of SEQ ID NO:3 from nucleotide 719 to nucleotide 1855, to a nucleotide sequence corresponding to the 3′ end of said sequence of SEQ ID NO:3 from nucleotide 719 to nucleotide 1855. Also preferably the polynucleotide isolated according to the above process comprises a nucleotide sequence corresponding to the cDNA sequence of SEQ ID NO:3 from nucleotide 779 to nucleotide 1855, and extending contiguously from a nucleotide sequence corresponding to the 5′ end of said sequence of SEQ ID NO:3 from nucleotide 779 to nucleotide 1855, to a nucleotide sequence corresponding to the 3′ end of said sequence of SEQ ID NO:3 from nucleotide 779 to nucleotide 1855.

In other embodiments, the present invention provides a composition comprising a protein, wherein said protein comprises an amino acid sequence selected from the group consisting of:

(a) the amino acid sequence of SEQ ID NO:4;

(b) the amino acid sequence of SEQ ID NO:4 from amino acid 1 to amino acid 27;

(c) fragments of the amino acid sequence of SEQ ID NO:4 comprising eight consecutive amino acids of SEQ ID NO:4;

(d) the amino acid sequence encoded by the cDNA insert of clone fj283_{—}11 deposited with the ATCC under accession number 98599; and

(e) the amino acid sequence encoded by the cDNA insert of clone fj283_{—}6 deposited with the ATCC under accession number xxxxx;

the protein being substantially free from other mammalian proteins. Preferably such protein comprises the amino acid sequence of SEQ ID NO:4 or the amino acid sequence of SEQ ID NO:4 from amino acid 1 to amino acid 27. In further preferred embodiments, the present invention provides a protein comprising a fragment of the amino acid sequence of SEQ ID NO:4 having biological activity, the fragment preferably comprising eight (more preferably twenty, most preferably thirty) consecutive amino acids of SEQ ID NO:4, or a protein comprising a fragment of the amino acid sequence of SEQ ID NO:4 having biological activity, the fragment comprising the amino acid sequence from amino acid 184 to amino acid 193 of SEQ ID NO:4.

In one embodiment, the present invention provides a composition comprising an isolated polynucleotide selected from the group consisting of:

(a) a polynucleotide comprising the nucleotide sequence of SEQ ID NO:198;

(b) a polynucleotide comprising the nucleotide sequence of SEQ ID NO:198 from nucleotide 982 to nucleotide 2118;

(c) a polynucleotide comprising the nucleotide sequence of SEQ ID NO:198 from nucleotide 1042 to nucleotide 2118;

(d) a polynucleotide comprising the nucleotide sequence of SEQ ID NO:198 from nucleotide 621 to nucleotide 1248;

(e) a polynucleotide comprising the nucleotide sequence of the full-length protein coding sequence of clone fj283_{—}6 deposited with the ATCC under accession number 98988;

(f) a polynucleotide encoding the full-length protein encoded by the cDNA insert of clone fj283_{—}6 deposited with the ATCC under accession number 98988;

(g) a polynucleotide comprising the nucleotide sequence of a mature protein coding sequence of clone fj283_{—}6 deposited with the ATCC under accession number 98988;

(h) a polynucleotide encoding a mature protein encoded by the cDNA insert of clone fj283_{—}6 deposited with the ATCC under accession number 98988;

(i) a polynucleotide encoding a protein comprising the amino acid sequence of SEQ ID NO:4;

(j) a polynucleotide encoding a protein comprising a fragment of the amino acid sequence of SEQ ID NO:4 having biological activity, the fragment comprising eight consecutive amino acids of SEQ ID NO:4;

(k) a polynucleotide which is an allelic variant of a polynucleotide of (a)-(h) above;

(l) a polynucleotide which encodes a species homologue of the protein of (i) or (j) above ; and

(m) a polynucleotide that hybridizes under stringent conditions to any one of the polynucleotides specified in (a)-(j).

Preferably, such polynucleotide comprises the nucleotide sequence of SEQ ID NO:198 from nucleotide 982 to nucleotide 2118; the nucleotide sequence of SEQ ID NO:198 from nucleotide 1042 to nucleotide 2118; the nucleotide sequence of SEQ ID NO: 198 from nucleotide 621 to nucleotide 1248; the nucleotide sequence of the full-length protein coding sequence of clone fj283_{—}6 deposited with the ATCC under accession number 98988; or the nucleotide sequence of a mature protein coding sequence of clone fj283_{—}6 deposited with the ATCC under accession number 98988. In other preferred embodiments, the polynucleotide encodes the full-length or a mature protein encoded by the cDNA insert of clone fj283_{—}6 deposited with the ATCC under accession number 98988. In further preferred embodiments, the present invention provides a polynucleotide encoding a protein comprising a fragment of the amino acid sequence of SEQ ID NO:4 having biological activity, the fragment preferably comprising eight (more preferably twenty, most preferably thirty) consecutive amino acids of SEQ ID NO:4, or a polynucleotide encoding a protein comprising a fragment of the amino acid sequence of SEQ ID NO:4 having biological activity, the fragment comprising the amino acid sequence from amino acid 184 to amino acid 193 of SEQ ID NO:4.

Other embodiments provide the gene corresponding to the cDNA sequence of SEQ ID NO:198.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:198, but excluding the poly(A) tail at the 3′ end of SEQ ID NO:198; and

(ab) the nucleotide sequence of the cDNA insert of clone fj283_{—}6 deposited with the ATCC under accession number 98988; and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:198, but excluding the poly(A) tail at the 3′ end of SEQ ID NO:198; and

(bb) the nucleotide sequence of the cDNA insert of clone fj283_{—}6 deposited with the ATCC under accession number 98988; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:198, and extending contiguously from a nucleotide sequence corresponding to the 5′ end of SEQ ID NO:198 to a nucleotide sequence corresponding to the 3′ end of SEQ ID NO:198, but excluding the poly(A) tail at the 3′ end of SEQ ID NO:198. Also preferably the polynucleotide isolated according to the above process comprises a nucleotide sequence corresponding to the cDNA sequence of SEQ ID NO:198 from nucleotide 982 to nucleotide 2118, and extending contiguously from a nucleotide sequence corresponding to the 5′ end of said sequence of SEQ ID NO:198 from nucleotide 982 to nucleotide 2118, to a nucleotide sequence corresponding to the 3′ end of said sequence of SEQ ID NO:198 from nucleotide 982 to nucleotide 2118. Also preferably the polynucleotide isolated according to the above process comprises a nucleotide sequence corresponding to the cDNA sequence of SEQ ID NO:198 from nucleotide 1042 to nucleotide 2118, and extending contiguously from a nucleotide sequence corresponding to the 5′ end of said sequence of SEQ ID NO:198 from nucleotide 1042 to nucleotide 2118, to a nucleotide sequence corresponding to the 3′ end of said sequence of SEQ ID NO:198 from nucleotide 1042 to nucleotide 2118. Also preferably the polynucleotide isolated according to the above process comprises a nucleotide sequence corresponding to the cDNA sequence of SEQ ID NO:198 from nucleotide 621 to nucleotide 1248, and extending contiguously from a nucleotide sequence corresponding to the 5′ end of said sequence of SEQ ID NO:198 from nucleotide 621 to nucleotide 1248, to a nucleotide sequence corresponding to the 3′ end of said sequence of SEQ ID NO:198 from nucleotide 621 to nucleotide 1248.

In one embodiment, the present invention provides a composition comprising an isolated polynucleotide selected from the group consisting of:

(a) a polynucleotide comprising the nucleotide sequence of SEQ ID NO:5;

(b) a polynucleotide comprising the nucleotide sequence of SEQ ID NO:5 from nucleotide 259 to nucleotide 624;

(c) a polvnucleotide comprising the nucleotide sequence of the full-length protein coding sequence of clone fk317_{—}3 deposited with the ATCC under accession number 98599;

(d) a polynucleotide encoding the full-length protein encoded by the cDNA insert of clone fk317_{—}3 deposited with the ATCC under accession number 98599;

(e) a polynucleotide comprising the nucleotide sequence of a mature protein coding sequence of clone fk317_{—}3 deposited with the ATCC under accession number 98599;

(f) a polynucleotide encoding a mature protein encoded by the cDNA insert of clone fk317_{—}3 deposited with the ATCC under accession number 98599;

(g) a polynucleotide encoding a protein comprising the amino acid sequence of SEQ ID NO:6;

(h) a polynucleotide encoding a protein comprising a fragment of the amino acid sequence of SEQ ID NO:6 having biological activity, the fragment comprising eight consecutive amino acids of SEQ ID NO:6;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:5 from nucleotide 259 to nucleotide 624; the nucleotide sequence of the full-length protein coding sequence of clone fk317_{—}3 deposited with the ATCC under accession number 98599; or the nucleotide sequence of a mature protein coding sequence of clone fk317_{—}3 deposited with the ATCC under accession number 98599. In other preferred embodiments, the polynucleotide encodes the full-length or a mature protein encoded by the cDNA insert of clone fk317_{—}3 deposited with the ATCC under accession number 98599. In yet other preferred embodiments, the present invention provides a polynucleotide encoding a protein comprising the amino acid sequence of SEQ ID NO:6 from amino acid 1 to amino acid 72. In further preferred embodiments, the present invention provides a polynucleotide encoding a protein comprising a fragment of the amino acid sequence of SEQ ID NO:6 having biological activity, the fragment preferably comprising eight (more preferably twenty, most preferably thirty) consecutive amino acids of SEQ ID NO:6, or a polynucleotide encoding a protein comprising a fragment of the amino acid sequence of SEQ ID NO:6 having biological activity, the fragment comprising the amino acid sequence from amino acid 56 to amino acid 65 of SEQ ID NO:6.

Other embodiments provide the gene corresponding to the cDNA sequence of SEQ ID NO:5.

Further embodiments of the invention provide isolated polynucleotides produced according to a process selected from the group consisting of:

(a) a process comprising the steps of:

(i) preparing one ormore polynucleotide probes thathybridize in 6×SSC at 65 degrees C to a nucleotide sequence selected from the group consisting of:

(aa) SEQ ID NO:5, but excluding the poly(A) tail at the 3′ end of SEQ ID NO:5; and

(ab) the nucleotide sequence of the cDNA insert of clone fk317_{—}3 deposited with the ATCC under accession number 98599; and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:5, but excluding the poly(A) tail at the 3′ end of SEQ ID NO:5; and

(bb) the nucleotide sequence of the cDNA insert of clone fk317_{—}3 deposited with the ATCC under accession number 98599; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:5, and extending contiguously from a nucleotide sequence corresponding to the 5′ end of SEQ ID NO:5 to a nucleotide sequence corresponding to the 3′ end of SEQ ID NO:5, but excluding the poly(A) tail at the 3′ end of SEQ ID NO:5. Also preferably the polynucleotide isolated according to the above process comprises a nucleotide sequence corresponding to the cDNA sequence of SEQ ID NO:5 from nucleotide 259 to nucleotide 624, and extending contiguously from a nucleotide sequence corresponding to the 5′ end of said sequence of SEQ ID NO:5 from nucleotide 259 to nucleotide 624, to a nucleotide sequence corresponding to the 3′ end of said sequence of SEQ ID NO:5 from nucleotide 259 to nucleotide 624.

In other embodiments, the present invention provides a composition comprising a protein, wherein said protein comprises an amino acid sequence selected from the group consisting of:

(a) the amino acid sequence of SEQ ID NO:6;

(b) the amino acid sequence of SEQ ID NO:6 from amino acid 1 to amino acid 72;

(c) fragments of the amino acid sequence of SEQ ID NO:6 comprising eight consecutive amino acids of SEQ ID NO:6; and

(d) the amino acid sequence encoded by the cDNA insert of clone fk317_{—}3 deposited with the ATCC under accession number 98599;

the protein being substantially free from other mammalian proteins. Preferably such protein comprises the amnino acid sequence of SEQ ID NO:6 or the amino acid sequence of SEQ ID NO:6 from amino acid 1 to amino acid 72. In further preferred embodiments, the present invention provides a protein comprising a fragment of the amino acid sequence of SEQ ID NO:6 having biological activity, the fragment preferably comprising eight (more preferably twenty, most preferably thirty) consecutive amino acids of SEQ ID NO:6, or a protein comprising a fragment of the amino acid sequence of SEQ ID NO:6 having biological activity, the fragment comprising the amino acid sequence from amino acid 56 to amino acid 65 of SEQ ID NO:6.

In one embodiment, the present invention provides a composition comprising an isolated polynucleotide selected from the group consisting of:

(a) a polynucleotide comprising the nucleotide sequence of SEQ ID NO:7;

(b) a polynucleotide comprising the nucleotide sequence of SEQ ID NO:7 from nucleotide 357 to nucleotide 578;

(c) a polynucleotide comprising the nucleotide sequence of SEQ ID NO:7 from nucleotide 471 to nucleotide 578;

(d) a polynucleotide comprising the nucleotide sequence of the full-length protein coding sequence of clone k213_{—}2x deposited with the ATCC under accession number 98599;

(e) a polynucleotide encoding the full-length protein encoded by the cDNA insert of clone k213_{—}2x deposited with the ATCC under accession number 98599;

(f) a polynucleotide comprising the nucleotide sequence of a mature protein coding sequence of clone k213_{—}2x deposited with the ATCC under accession number 98599;

(g) a polynucleotide encoding a mature protein encoded by the cDNA insert of clone k213_{—}2x deposited with the ATCC under accession number 98599;

(h) a polynucleotide encoding a protein comprising the amino acid sequence of SEQ ID NO:8;

(i) a polynucleotide encoding a protein comprising a fragment of the amino acid sequence of SEQ ID NO:8 having biological activity, the fragment comprising eight consecutive amino acids of SEQ ID NO:8;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:7 from nucleotide 357 to nucleotide 578; the nucleotide sequence of SEQ ID NO:7 from nucleotide 471 to nucleotide 578; the nucleotide sequence of the full-length protein coding sequence of clone k213_{—}2x deposited with the ATCC under accession number 98599; or the nucleotide sequence of a mature protein coding sequence of clone k213_{—}2x deposited with the ATCC under accession number 98599. In other preferred embodiments, the polynucleotide encodes the full-length or a mature protein encoded by the cDNA insert of clone k213_{—}2x deposited with the ATCC under accession number 98599. In further preferred embodiments, the present invention provides a polynucleotide encoding a protein comprising a fragment of the amino acid sequence of SEQ ID NO:8 having biological activity, the fragment preferably comprising eight (more preferably twenty, most preferably thirty) consecutive amino acids of SEQ ID NO:8, or a polynucleotide encoding a protein comprising a fragment of the amino acid sequence of SEQ ID NO:8 having biological activity, the fragment comprising the amino acid sequence from amino acid 32 to amino acid 41 of SEQ ID NO:8.

Other embodiments provide the gene corresponding to the cDNA sequence of SEQ ID NO:7.

Further embodiments of the invention provide isolated polynucleotides produced according to a process selected from the group consisting of:

(a) a process comprising the steps of:

(i) preparing one or more polynudeotide probes that hybridize in 6×SSC at 65 degrees C to a nucleotide sequence selected from the group consisting of:

(aa) SEQ ID NO:7, but excluding the poly(A) tail at the 3′ end of SEQ ID NO:7; and

(ab) the nucleotide sequence of the cDNA insert of clone k213_{—}2x deposited with the ATCC under accession number 98599; and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:7, but excluding the poly(A) tail at the 3′ end of SEQ ID NO:7; and

(bb) the nucleotide sequence of the cDNA insert of clone k213_{—}2x deposited with the ATCC under accession number 98599; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:7, and extending contiguously from a nucleotide sequence corresponding to the 5′ end of SEQ ID NO:7 to a nucleotide sequence corresponding to the 3′ end of SEQ ID NO:7, but excluding the poly(A) tail at the 3′ end of SEQ ID NO:7. Also preferably the polynucleotide isolated according to the above process comprises a nucleotide sequence corresponding to the cDNA sequence of SEQ ID NO:7 from nucleotide 357 to nucleotide 578, and extending contiguously from a nucleotide sequence corresponding to the 5′ end of said sequence of SEQ ID NO:7 from nucleotide 357 to nucleotide 578, to a nucleotide sequence corresponding to the 3′ end of said sequence of SEQ ID NO:7 from nucleotide 357 to nucleotide 578. Also preferably the polynucleotide isolated according to the above process comprises a nucleotide sequence corresponding to the cDNA sequence of SEQ ID NO:7 from nucleotide 471 to nucleotide 578, and extending contiguously from a nucleotide sequence corresponding to the 5′ end of said sequence of SEQ ID NO:7 from nucleotide 471 to nucleotide 578, to a nucleotide sequence corresponding to the 3′ end of said sequence of SEQ ID NO:7 from nucleotide 471 to nucleotide 578.

In other embodiments, the present invention provides a composition comprising a protein, wherein said protein comprises an amino acid sequence selected from the group consisting of:

(a) the amino acid sequence of SEQ ID NO:8;

(b) fragments of the amino acid sequence of SEQ ID NO:8 comprising eight consecutive amino acids of SEQ ID NO:8; and

(c) the amino acid sequence encoded by the cDNA insert of clone k213_{—}2x deposited with the ATCC under accession number 98599;

the protein being substantially free from other mammalian proteins. Preferably such protein comprises the amino acid sequence of SEQ ID NO:8. In further preferred embodiments, the present invention provides a protein comprising a fragment of the amino acid sequence of SEQ ID NO:8 having biological activity, the fragment preferably comprising eight (more preferably twenty, most preferably thirty) consecutive amino acids of SEQ ID NO:8, or a protein comprising a fragment of the amino acid sequence of SEQ ID NO:8 having biological activity, the fragment comprising the amino acid sequence from amino acid 32 to amino acid 41 of SEQ ID NO:8.

In one embodiment, the present invention provides a composition comprising an isolated polynucleotide selected from the group consisting of:

(a) a polynucleotide comprising the nucleotide sequence of SEQ ID NO:9;

(b) a polynucleotide comprising the nucleotide sequence of SEQ ID NO:9 from nucleotide 332 to nucleotide 598;

(c) a polynucleotide comprising the nucleotide sequence of SEQ ID NO:9 from nucleotide 458 to nucleotide 598;

(d) a polynucleotide comprising the nucleotide sequence of the full-length protein coding sequence of clone na316_{—}1 deposited with the ATCC under accession number 98599;

(e) a polynucleotide encoding the full-length protein encoded by the cDNA insert of clone na316_{—}1 deposited with the ATCC under accession number 98599;

(f) a polynucleotide comprising the nucleotide sequence of a mature protein coding sequence of clone na316_{—}6deposited with the ATCC under accession number 98599;

(g) a polynucleotide encoding a mature protein encoded by the cDNA insert of clone na316_{—}1 deposited with the ATCC under accession number 98599;

(h) a polynucleotide encoding a protein comprising the amino acid sequence of SEQ ID NO:10;

(i) a polynucleotide encoding a protein comprising a fragment of the amino acid sequence of SEQ ID NO:10 having biological activity, the fragment comprising eight consecutive amino acids of SEQ ID NO:10;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:9 from nucleotide 332 to nucleotide 598; the nucleotide sequence of SEQ ID NO:9 from nucleotide 458 to nucleotide 598; the nucleotide sequence of the full-length protein coding sequence of clone na316_{—}6 deposited with the ATCC under accession number 98599; or the nucleotide sequence of a mature protein coding sequence of clone na316_{—}1 deposited with the ATCC under accession number 98599. In other preferred embodiments, the polynucleotide encodes the full-length or a mature protein encoded by the cDNA insert of clone na316_{—}1 deposited with the ATCC under accession number 98599. In further preferred embodiments, the present invention provides a polynucleotide encoding a protein comprising a fragment of the amino acid sequence of SEQ ID NO:10 having biological activity, the fragment preferably comprising eight (more preferably twenty, most preferably thirty) consecutive amino acids of SEQ ID NO:10, or a polynucleotide encoding a protein comprising a fragment of the amino acid sequence of SEQ ID NO:10 having biological activity, the fragment comprising the amino acid sequence from amino acid 39 to amino acid 48 of SEQ ID NO:10.

Other embodiments provide the gene corresponding to the cDNA sequence of SEQ ID NO:9.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:9, but excluding the poly(A) tail at the 3′ end of SEQ ID NO:9; and

(ab) the nucleotide sequence of the cDNA insert of clone na316₋1 deposited with the ATCC under accession number 98599; and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:9, but excluding the poly(A) tail at the 3′ end of SEQ ID NO:9; and

(bb) the nucleotide sequence of the cDNA insert of clone na316_{—}1 deposited with the ATCC under accession number 98599; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:9, and extending contiguously from a nucleotide sequence corresponding to the 5′ end of SEQ ID NO:9 to a nucleotide sequence corresponding to the 3′ end of SEQ ID NO:9 , but excluding the poly(A) tail at the 3′ end of SEQ ID NO:9. Also preferably the polynucleotide isolated according to the above process comprises a nucleotide sequence corresponding to the cDNA sequence of SEQ ID NO:9 from nucleotide 332 to nucleotide 598, and extending contiguously from a nucleotide sequence corresponding to the 5′ end of said sequence of SEQ ID NO:9 from nucleotide 332 to nucleotide 598, to a nucleotide sequence corresponding to the 3′ end of said sequence of SEQ ID NO:9 from nucleotide 332 to nucleotide 598. Also preferably the polynucleotide isolated according to the above process comprises a nucleotide sequence corresponding to the cDNA sequence of SEQ ID NO:9 from nucleotide 458 to nucleotide 598, and extending contiguously from a nucleotide sequence corresponding to the 5′ end of said sequence of SEQ ID NO:9 from nucleotide 458 to nucleotide 598, to a nucleotide sequence corresponding to the 3′ end of said sequence of SEQ ID NO:9 from nucleotide 458 to nucleotide 598.

In other embodiments, the present invention provides a composition comprising a protein, wherein said protein comprises an amino acid sequence selected from the group consisting of:

(a) the amino acid sequence of SEQ ID NO:10;

(b) fragments of the amino acid sequence of SEQ ID NO: 10 comprising eight consecutive amino acids of SEQ ID NO:10; and

(c) the amino acid sequence encoded by the cDNA insert of clone na316_{—}1 deposited with the ATCC under accession number 98599;

the protein being substantially free from other mammalian proteins. Preferably such protein comprises the amino acid sequence of SEQ ID NO:10. In further preferred embodiments, the present invention provides a protein comprising a fragment of the amino acid sequence of SEQ ID NO: 10 having biological activity, the fragment preferably comprising eight (more preferably twenty, most preferably thirty) consecutive amino acids of SEQ ID NO:10, or a protein comprising a fragment of the amino acid sequence of SEQ ID NO:10 having biological activity, the fragment comprising the amino acid sequence from amino acid 39 to amino acid 48 of SEQ ID NO:10.

In one embodiment, the present invention provides a composition comprising an isolated polynucleotide selected from the group consisting of:

(a) a polynucleotide comprising the nucleotide sequence of SEQ ID NO:11;

(b) a polynucleotide comprising the nucleotide sequence of SEQ ID NO:11 from nucleotide 354 to nucleotide 986;

(c) a polynucleotide comprising the nucleotide sequence of SEQ ID NO:11 from nucleotide 408 to nucleotide 986;

(d) a polynucleotide comprising the nucleotide sequence of the full-length protein coding sequence of clone nf93_{—}20 deposited with the ATCC under accession number 98599;

(e) a polynucleotide encoding the full-length protein encoded by the cDNA insert of clone nf93_{—}20 deposited with the ATCC under accession number 98599;

(f) a polynucleotide comprising the nucleotide sequence of a mature protein coding sequence of clone nf93_{—}20 deposited with the ATCC under accession number 98599;

(g) a polynucleotide encoding a mature protein encoded by the cDNA insert of clone nf93_{—}20 deposited with the ATCC under accession number 98599;

(h) a polynucleotide encoding a protein comprising the amino acid sequence of SEQ ID NO:12;

(i) a polynucleotide encoding a protein comprising a fragment of the amino acid sequence of SEQ ID NO:12 having biological activity, the fragment comprising eight consecutive amino acids of SEQ ID NO:12;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:1l from nucleotide 354 to nucleotide 986; the nucleotide sequence of SEQ ID NO:11 from nucleotide 408 to nucleotide 986; the nucleotide sequence of the full-length protein coding sequence of clone nf93_{—}20 deposited with the ATCC under accession number 98599; or the nucleotide sequence of a mature protein coding sequence of clone nf93_{—}20 deposited with the ATCC under accession number 98599. In other preferred embodiments, the polynucleotide encodes the full-length or a mature protein encoded by the cDNA insert of clone nf93_{—}20 deposited with the ATCC under accession number 98599. In further preferred embodiments, the present invention provides a polynucleotide encoding a protein comprising a fragment of the amino acid sequence of SEQ ID NO:12 having biological activity, the fragment preferably comprising eight (more preferably twenty, most preferably thirty) consecutive amino acids of SEQ ID NO:12, or a polynucleotide encoding a protein comprising a fragment of the amino acid sequence of SEQ ID NO:12 having biological activity, the fragment comprising the amino acid sequence from amino acid 100 to amino acid 109 of SEQ ID NO:12.

Other embodiments provide the gene corresponding to the cDNA sequence of SEQ ID NO:11.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:11, but excluding the poly(A) tail at the 3′ end of SEQ ID NO:11; and

(ab) the nucleotide sequence of the cDNA insert of clone nf93_{—}20 deposited with the ATCC under accession number 98599; and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:11, but excluding the poly(A) tail at the 3′ end of SEQ ID NO:11; and

(bb) the nucleotide sequence of the cDNA insert of clone nf93_{—}20 deposited with the ATCC under accession number 98599; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:11, and extending contiguously from a nucleotide sequence corresponding to the 5′ end of SEQ ID NO:11 to a nucleotide sequence corresponding to the 3′ end of SEQ ID NO:11, but excluding the poly(A) tail at the 3′ end of SEQ ID NO:11. Also preferably the polynucleotide isolated according to the above process comprises a nucleotide sequence corresponding to the cDNA sequence of SEQ ID NO:11 from nucleotide 354 to nucleotide 986, and extending contiguously from a nucleotide sequence corresponding to the 5′ end of said sequence of SEQ ID NO:11 from nucleotide 354 to nucleotide 986, to a nucleotide sequence corresponding to the 3′ end of said sequence of SEQ ID NO:11 from nucleotide 354 to nucleotide 986. Also preferably the polynucleotide isolated according to the above process comprises a nucleotide sequence corresponding to the cDNA sequence of SEQ ID NO:11 from nucleotide 408 to nucleotide 986, and extending contiguously from a nucleotide sequence corresponding to the 5′ end of said sequence of SEQ ID NO:11 from nucleotide 408 to nucleotide 986, to a nucleotide sequence corresponding to the 3′ end of said sequence of SEQ ID NO:11 from nucleotide 408 to nucleotide 986.

In other embodiments, the present invention provides a composition comprising a protein, wherein said protein comprises an amino acid sequence selected from the group consisting of:

(a) the amino acid sequence of SEQ ID NO:12;

(b) fragments of the amino acid sequence of SEQ ID NO: 12 comprising eight consecutive amino acids of SEQ ID NO:12; and

(c) the amino acid sequence encoded by the cDNA insert of clone nf93_{—}20 deposited with the ATCC under accession number 98599;

the protein being substantially free from other mammalian proteins. Preferably such protein comprises the amino acid sequence of SEQ ID NO:12. In further preferred embodiments, the present invention provides a protein comprising a fragment of the amino acid sequence of SEQ ID NO:12 having biological activity, the fragment preferably comprising eight (more preferably twenty, most preferably thirty) consecutive amino acids of SEQ ID NO:12, or a protein comprising a fragment of the amino acid sequence of SEQ ID NO:12 having biological activity, the fragment comprising the amino acid sequence from amino acid 100 to amino acid 109 of SEQ ID NO:12.

In one embodiment, the present invention provides a composition comprising an isolated polynucleotide selected from the group consisting of:

(a) a polynucleotide comprising the nucleotide sequence of SEQ ID NO:13;

(b) a polynucleotide comprising the nucleotide sequence of SEQ ID NO:13 from nucleotide 301 to nucleotide 1821;

(c) a polynucleotide comprising the nucleotide sequence of SEQ ID NO:13 from nucleotide 1381 to nucleotide 1821;

(d) a polynucleotide comprising the nucleotide sequence of the full-length protein coding sequence of clone np164_{—}1 deposited with the ATCC under accession number 98599;

(e) a polynucleotide encoding the full-length protein encoded by the cDNA insert of clone np164_{—}1 deposited with the ATCC under accession number 98599;

(f) a polynucleotide comprising the nucleotide sequence of a mature protein coding sequence of clone np164_{—}1 deposited with the ATCC under accession number 98599;

(g) a polynucleotide encoding a mature protein encoded by the cDNA insert of clone np164_{—}1 deposited with the ATCC under accession number 98599;

(h) a polynucleotide encoding a protein comprising the amnino acid sequence of SEQ ID NO:14;

(i) a polynucleotide encoding a protein comprising a fragment of the amino acid sequence of SEQ ID NO:14 having biological activity, the fragment comprising eight consecutive amino acids of SEQ ID NO:14;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:13 from nucleotide 301 to nucleotide 1821; the nucleotide sequence of SEQ ID NO:13 from nucleotide 1381 to nucleotide 1821; the nucleotide sequence of the full-length protein coding sequence of clone np164_{—}1 deposited with the ATCC under accession number 98599; or the nucleotide sequence of a mature protein coding sequence of clone np164_{—}1 deposited with the ATCC under accession number 98599. In other preferred embodiments, the polynucleotide encodes the full-length or a mature protein encoded by the cDNA insert of clone np164_{—}1 deposited with the ATCC under accession number 98599. In further preferred embodiments, the present invention provides a polynucleotide encoding a protein comprising a fragment of the amino acid sequence of SEQ ID NO:14 having biological activity, the fragment preferably comprising eight (more preferably twenty, most preferably thirty) consecutive amino acids of SEQ ID NO:14, or a polynucleotide encoding a protein comprising a fragment of the amino acid sequence of SEQ ID NO:14 having biological activity, the fragment comprising the amino acid sequence from amnino acid 248 to amino acid 257 of SEQ ID NO:14.

Other embodiments provide the gene corresponding to the cDNA sequence of SEQ ID NO:13.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:13, but excluding the poly(A) tail at the 3′ end of SEQ ID NO:13; and

(ab) the nucleotide sequence of the cDNA insert of clone np164_{—}1 deposited with the ATCC under accession number 98599; and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:13, but excluding the poly(A) tail at the 3′ end of SEQ ID NO:13; and

(bb) the nucleotide sequence of the cDNA insert of clone np164_{—}1 deposited with the ATCC under accession number 98599; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:13, and extending contiguously from a nucleotide sequence corresponding to the 5′ end of SEQ ID NO:13 to a nucleotide sequence corresponding to the 3′ end of SEQ ID NO:13, but excluding the poly(A) tail at the 3′ end of SEQ ID NO:13. Also preferably the polynucleotide isolated according to the above process comprises a nucleotide sequence corresponding to the cDNA sequence of SEQ ID NO:13 from nucleotide 301 to nucleotide 1821, and extending contiguously from a nucleotide sequence corresponding to the 5′ end of said sequence of SEQ ID NO:13 from nucleotide 301 to nucleotide 1821, to a nucleotide sequence corresponding to the 3′ end of said sequence of SEQ ID NO:13 from nucleotide 301 to nucleotide 1821. Also preferably the polynucleotide isolated according to the above process comprises a nucleotide sequence corresponding to the cDNA sequence of SEQ ID NO:13 from nucleotide 1381 to nucleotide 1821, and extending contiguously from a nucleotide sequence corresponding to the 5′ end of said sequence of SEQ ID NO:13 from nucleotide 1381 to nucleotide 1821, to a nucleotide sequence corresponding to the 3′ end of said sequence of SEQ ID NO:13 from nucleotide 1381 to nucleotide 1821.

In other embodiments, the present invention provides a composition comprising a protein, wherein said protein comprises an amino acid sequence selected from the group consisting of:

(a) the amino acid sequence of SEQ ID NO:14;

(b) fragments of the amino acid sequence of SEQ ID NO: 14 comprising eight consecutive amino acids of SEQ ID NO:14; and

(c) the amino acid sequence encoded by the cDNA insert of clone np164_{—}1 deposited with the ATCC under accession number 98599;

the protein being substantially free from other mammalian proteins. Preferably such protein comprises the amino acid sequence of SEQ ID NO:14. In further preferred embodiments, the present invention provides a protein comprising a fragment of the amino acid sequence of SEQ ID NO:14 having biological activity, the fragment preferably comprising eight (more preferably twenty, most preferably thirty) consecutive amino acids of SEQ ID NO:14, or a protein comprising a fragment of the amino acid sequence of SEQ ID NO:14 having biological activity, the fragment comprising the amino acid sequence from amino acid 248 to amino acid 257 of SEQ ID NO:14.

In one embodiment, the present invention provides a composition comprising an isolated polynucleotide selected from the group consisting of:

(a) a polynucleotide comprising the nucleotide sequence of SEQ ID NO:15;

(b) a polynucleotide comprising the nucleotide sequence of SEQ ID NO:15 from nucleotide 148 to nucleotide 537;

(c) a polynucleotide comprising the nucleotide sequence of the full-length protein coding sequence of clone pe204_{—}1 deposited with the ATCC under accession number 98599;

(d) a polynucleotide encoding the full-length protein encoded by the cDNA insert of clone pe204_{—}1 deposited with the ATCC under accession number 98599;

(e) a polynucleotide comprising the nucleotide sequence of a mature protein coding sequence of clone pe204_{—}1 deposited with the ATCC under accession number 98599;

(f) a polynucleotide encoding a mature protein encoded by the cDNA insert of clone pe204_{—}1 deposited with the ATCC under accession number 98599;

(g) a polynucleotide encoding a protein comprising the amino acid sequence of SEQ ID NO:16;

(h) a polynucleotide encoding a protein comprising a fragment of the amino acid sequence of SEQ ID NO:16 having biological activity, the fragment comprising eight consecutive amino acids of SEQ ID NO:16;

(i) a polynucleotide which is an allelic variant of a polynucleotide of (a)-(h) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:15 from nucleotide 148 to nucleotide 537; the nucleotide sequence of the full-length protein coding sequence of clone pe204_{—}1 deposited with the ATCC under accession number 98599; or the nucleotide sequence of a mature protein coding sequence of clone pe204_{—}1 deposited with the ATCC under accession number 98599. In other preferred embodiments, the polynucleotide encodes the full-length or a mature protein encoded by the cDNA insert of clone pe204_{—}1 deposited with the ATCC under accession number 98599. In further preferred embodiments, the present invention provides a polynucleotide encoding a protein comprising a fragment of the amino acid sequence of SEQ ID NO:16 having biological activity, the fragmentpreferably comprising eight (more preferably twenty, most preferably thirty) consecutive amino acids of SEQ ID NO:16, or a polynucleotide encoding a protein comprising a fragment of the amino acid sequence of SEQ ID NO:16 having biological activity, the fragment comprising the amino acid sequence from amino acid 60 to amino acid 69 of SEQ ID NO: 16.

Other embodiments provide the gene corresponding to the cDNA sequence of SEQ ID NO:15.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO: 15, but excluding the poly(A) tail at the 3′ end of SEQ ID NO:15; and

(ab) the nucleotide sequence of the cDNA insert of clone pe204_{—}1 deposited with the ATCC under accession number 98599; and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:15, but excluding the poly(A) tail at the 3′ end of SEQ ID NO:15; and

(bb) the nucleotide sequence of the cDNA insert of clone pe204_{—}1 deposited with the ATCC under accession number 98599; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:15, and extending contiguously from a nucleotide sequence corresponding to the 5′ end of SEQ ID NO:15 to a nucleotide sequence corresponding to the 3′ end of SEQ ID NO:15 , but excluding the poly(A) tail at the 3′ end of SEQ ID NO:15. Also preferably the polynucleotide isolated according to the above process comprises a nucleotide sequence corresponding to the cDNA sequence of SEQ ID NO:15 from nucleotide 148 to nucleotide 537, and extending contiguously from a nucleotide sequence corresponding to the 5′ end of said sequence of SEQ ID NO: 15 from nucleotide 148 to nucleotide 537, to a nucleotide sequence corresponding to the 3′ end of said sequence of SEQ ID NO:15 from nucleotide 148 to nucleotide 537.

In other embodiments, the present invention provides a composition comprising a protein, wherein said protein comprises an amino acid sequence selected from the group consisting of:

(a) the amino acid sequence of SEQ ID NO:16;

(b) fragments of the amino acid sequence of SEQ IDNO:16 comprising eight consecutive amino acids of SEQ ID NO:16; and

(c) the armino acid sequence encoded by the cDNA insert of clone pe204_{—}1 deposited with the ATCC under accession number 98599;

the protein being substantially free from other mamnmalian proteins. Preferably such protein comprises the amino acid sequence of SEQ ID NO:16. In further preferred embodiments, the present invention provides a protein comprising a fragment of the amino acid sequence of SEQ ID NO:16 having biological activity, the fragment preferably comprising eight (more preferably twenty, most preferably thirty) consecutive amino acids of SEQ ID NO:16, or a protein comprising a fragment of the amino acid sequence of SEQ ID NO:16 having biological activity, the fragment comprising the amino acid sequence from amino acid 60 to amino acid 69 of SEQ ID NO:16.

In one embodiment, the present invention provides a composition comprising an isolated polynucleotide selected from the group consisting of:

(a) a polynucleotide comprising the nucleotide sequence of SEQ ID NO:17;

(b) a polynucleotide comprising the nucleotide sequence of SEQ ID NO:17 from nucleotide 24 to nucleotide 1109;

(c) a polynucleotide comprising the nucleotide sequence of SEQ ID NO:17 from nucleotide 1050 to nucleotide 1109;

(d) a polynucleotide comprising the nucleotide sequence of the full-length protein coding sequence of clone ya1_{—}1 deposited with the ATCC under accession number 98599;

(e) a polynucleotide encoding the full-length protein encoded by the cDNA insert of clone ya1_{—}1 deposited with the ATCC under accession number 98599;

(f) a polynucleotide comprising the nucleotide sequence of a mature protein coding sequence of clone ya1_{—}1 deposited with the ATCC under accession number 98599;

(g) a polynucleotide encoding a mature protein encoded by the cDNA insert of clone ya1_{—}1 deposited with the ATCC under accession number 98599;

(h) a polynucleotide encoding a protein comprising the amino acid sequence of SEQ ID NO:18;

(i) a polynucleotide encoding a protein comprising a fragment of the amino acid sequence of SEQ ID NO:18 having biological activity, the fragment comprising eight consecutive amino acids of SEQ ID NO:18;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:17 from nucleotide 24 to nucleotide 1109; the nucleotide sequence of SEQ ID NO:17 from nucleotide 1050 to nucleotide 1109; the nucleotide sequence of the full-length protein coding sequence of clone ya1_{—}1 deposited with the ATCC under accession number 98599; or the nucleotide sequence of a mature protein coding sequence of clone ya1_{—}1 deposited with the ATCC under accession number 98599. In other preferred embodiments, the polynucleotide encodes the full-length or a mature protein encoded by the cDNA insert of clone ya1_{—}1 deposited with the ATCC under accession number 98599. In further preferred embodiments, the present invention provides a polynucleotide encoding a protein comprising a fragment of the amino acid sequence of SEQ ID NO:18 having biological activity, the fragment preferably comprising eight (more preferably twenty, most preferably thirty) consecutive amino acids of SEQ ID NO:18, or a polynucleotide encoding a protein comprising a fragment of the amino acid sequence of SEQ ID NO:18 having biological activity, the fragment comprising the amino acid sequence from amino acid 176 to amino acid 185 of SEQ ID NO:18.

Other embodiments provide the gene corresponding to the cDNA sequence of SEQ ID NO:17.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:17, but excluding the poly(A) tail at the 3′ end of SEQ ID NO:17; and

(ab) the nucleotide sequence of the cDNA insert of clone ya1_{—}1 deposited with the ATCC under accession number 98599; and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO: 17, but excluding the poly(A) tail at the 3′ end of SEQ ID NO:17; and

(bb) the nucleotide sequence of the cDNA insert of clone ya1_{—}1 deposited with the ATCC under accession number 98599; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:17, and extending contiguously from a nucleotide sequence corresponding to the 5′ end of SEQ ID NO:17 to a nucleotide sequence corresponding to the 3′ end of SEQ ID NO:17, but excluding the poly(A) tail at the 3′ end of SEQ ID NO:17. Also preferably the polynucleotide isolated according to the above process comprises a nucleotide sequence corresponding to the cDNA sequence of SEQ ID NO:17 from nucleotide 24 to nucleotide 1109, and extending contiguously from a nucleotide sequence corresponding to the 5′ end of said sequence of SEQ ID NO:17 from nucleotide 24 to nucleotide 1109, to a nucleotide sequence corresponding to the 3′ end of said sequence of SEQ ID NO:17 from nucleotide 24 to nucleotide 1109. Also preferably the polynucleotide isolated according to the above process comprises a nucleotide sequence corresponding to the cDNA sequence of SEQ ID NO:17 from nucleotide 1050 to nucleotide 1109, and extending contiguously from a nucleotide sequence corresponding to the 5′ end of said sequence of SEQ ID NO:17 from nucleotide 1050 to nucleotide 1109, to a nucleotide sequence corresponding to the 3′ end of said sequence of SEQ ID NO:17 from nucleotide 1050 to nucleotide 1109.

In other embodiments, the present invention provides a composition comprising a protein, wherein said protein comprises an amino acid sequence selected from the group consisting of:

(a) the amino acid sequence of SEQ ID NO:18;

(b) fragments of the amino acid sequence of SEQ ID NO:18 comprising eight consecutive amino acids of SEQ ID NO:18; and

(c) the amino acid sequence encoded by the cDNA insert of clone ya1_{—}1 deposited with the ATCC under accession number 98599;

the protein being substantially free from other mammalian proteins. Preferably such protein comprises the amino acid sequence of SEQ ID NO:18. In further preferred embodiments, the present invention provides a protein comprising a fragment of the amino acid sequence of SEQ ID NO:18 having biological activity, the fragment preferably comprising eight (more preferably twenty, most preferably thirty) consecutive amino acids of SEQ ID NO:18, or a protein comprising a fragment of the amino acid sequence of SEQ ID NO:18 having biological activity, the fragment comprising the amino acid sequence from amnino acid 176 to amino acid 185 of SEQ ID NO:18.

In one embodiment, the present invention provides a composition comprising an isolated polynucleotide selected from the group consisting of:

(a) a polynucleotide comprising the nucleotide sequence of SEQ ID NO:19;

(b) a polynucleotide comprising the nucleotide sequence of SEQ ID NO:19 from nucleotide 27 to nucleotide 734;

(c) a polynucleotide comprising the nucleotide sequence of SEQ ID NO:19 from nucleotide 270 to nucleotide 734;

(d) a polynucleotide comprising the nucleotide sequence of SEQ ID NO:19 from nucleotide 85 to nucleotide 1604;

(e) a polynucleotide comprising the nucleotide sequence of the full-length protein coding sequence of clone yb8_{—}1 deposited with the ATCC under accession number 98599;

(f) a polynucleotide encoding the full-length protein encoded by the cDNA insert of clone yb8_{—}1 deposited with the ATCC under accession number 98599;

(g) a polynucleotide comprising the nucleotide sequence of a mature protein coding sequence of clone yb8_{—}1 deposited with the ATCC under accession number 98599;

(h) a polynucleotide encoding a mature protein encoded by the cDNA insert of clone yb8_{—}1 deposited with the ATCC under accession number 98599;

(i) a polynucleotide encoding a protein comprising the amino acid sequence of SEQ ID NO:20;

(j) a polynucleotide encoding a protein comprising a fragment of the amino acid sequence of SEQ ID NO:20 having biological activity, the fragment comprising eight consecutive amino acids of SEQ ID NO:20;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:19 from nucleotide 27 to nucleotide 734; the nucleotide sequence of SEQ ID NO:19 from nucleotide 270 to nucleotide 734; the nucleotide sequence of SEQ ID NO:19 from nucleotide 85 tonucleotide 1604; the nucleotide sequence of the full-length protein coding sequence of clone yb8_{—}1 deposited with the ATCC under accession number 98599; or the nucleotide sequence of a mature protein coding sequence of clone yb8_{—}1 deposited with the ATCC under accession number 98599. In other preferred embodiments, the polynucleotide encodes the full-length or a mature protein encoded by the cDNA insert of clone yb8_{—}1 deposited with the ATCC under accession number 98599. In yet other preferred embodiments, the present invention provides a polynucleotide encoding a protein comprising the amino acid sequence of SEQ ID NO:20 from amino acid 70 to amino acid 236. In further preferred embodiments, the present invention provides a polynucleotide encoding a protein comprising a fragment of the amino acid sequence of SEQ ID NO:20 having biological activity, the fragment preferably comprising eight (more preferably twenty, most preferably thirty) consecutive amino acids of SEQ ID NO:20, or a polynucleotide encoding a protein comprising a fragment of the amino acid sequence of SEQ ID NO:20 having biological activity, the fragment comprising the amino acid sequence from amino acid 113 to amino acid 122 of SEQ ID NO:20.

Other embodiments provide the gene corresponding to the cDNA sequence of SEQ ID NO:19.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:19, but excluding the poly(A) tail at the 3′ end of SEQ ID NO:19; and

(ab) the nucleotide sequence of the cDNA insert of clone yb8_{—}1 deposited with the ATCC under accession number 98599; and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:19, but excluding the poly(A) tail at the 3′ end of SEQ ID NO:19; and

(bb) the nucleotide sequence of the cDNA insert of clone yb8_{—}1 deposited with the ATCC under accession number 98599; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:19, and extending contiguously from a nucleotide sequence corresponding to the 5′ end of SEQ ID NO:19 to a nucleotide sequence corresponding to the 3′ end of SEQ ID NO:19, but excluding the poly(A) tail at the 3′ end of SEQ ID NO:19. Also preferably the polynucleotide isolated according to the above process comprises a nucleotide sequence corresponding to the cDNA sequence of SEQ ID NO:19 from nucleotide 27 to nucleotide 734, and extending contiguously from a nucleotide sequence corresponding to the 5′ end of said sequence of SEQ ID NO:19 from nucleotide 27 to nucleotide 734, to a nucleotide sequence corresponding to the 3′ end of said sequence of SEQ ID NO:19 from nucleotide 27 to nucleotide 734. Also preferably the polynucleotide isolated according to the above process comprises a nucleotide sequence corresponding to the cDNA sequence of SEQ ID NO:19 from nucleotide 270 to nucleotide 734, and extending contiguously from a nucleotide sequence corresponding to the 5′ end of said sequence of SEQ ID NO:19 from nucleotide 270 to nucleotide 734, to a nucleotide sequence corresponding to the 3′ end of said sequence of SEQ ID NO:19 from nucleotide 270 to nucleotide 734. Also preferably the polynucleotide isolated according to the above process comprises a nucleotide sequence corresponding to the cDNA sequence of SEQ ID NO:19 from nucleotide 85 to nucleotide 1604, and extending contiguously from a nucleotide sequence corresponding to the 5′ end of said sequence of SEQ ID NO:19 from nucleotide 85 to nucleotide 1604, to a nucleotide sequence corresponding to the 3′ end of said sequence of SEQ ID NO:19 from nucleotide 85 to nucleotide 1604.

In other embodiments, the present invention provides a composition comprising a protein, wherein said protein comprises an amino acid sequence selected from the group consisting of:

(a) the amino acid sequence of SEQ ID NO:20;

(b) the amino acid sequence of SEQ ID NO:20 from amino acid 70 to amino acid 236;

(c) fragments of the amino acid sequence of SEQ ID NO:20 comprising eight consecutive amino acids of SEQ ID NO:20; and

(d) the amino acid sequence encoded by the cDNA insert of clone yb8_{—}1 deposited with the ATCC under accession number 98599;

the protein being substantially free from other mammalian proteins. Preferably such protein comprises the amino acid sequence of SEQ ID NO:20 or the amino acid sequence of SEQ ID NO:20 from amino acid 70 to amino acid 236. In further preferred embodiments, the present invention provides a protein comprising a fragment of the amino acid sequence of SEQ ID NO:20 having biological activity, the fragment preferably comprising eight (more preferably twenty, most preferably thirty) consecutive amino acids of SEQ ID NO:20, or a protein comprising a fragment of the amino acid sequence of SEQ ID NO:20 having biological activity, the fragment comprising the amino acid sequence from amino acid 113 to amino acid 122 of SEQ ID NO:20.

In one embodiment, the present invention provides a composition comprising an isolated polynucleotide selected from the group consisting of:

(a) a polynucleotide comprising the nucleotide sequence of SEQ ID NO:21;

(b) a polynucleotide comprising the nucleotide sequence of SEQ ID NO:21 from nucleotide 469 to nucleotide 609;

(c) a polynucleotide comprising the nucleotide sequence of SEQ ID NO:21 from nucleotide 574 to nucleotide 609;

(d) a polynucleotide comprising the nucleotide sequence of SEQ ID NO:21 from nucleotide 214 to nucleotide 369;

(e) a polynucleotide comprising the nucleotide sequence of the full-length protein coding sequence of clone am856_{—}3 deposited with the ATCC under accession number 98600;

(f) a polynucleotide encoding the full-length protein encoded by the cDNA insert of clone am856_{—}3 deposited with the ATCC under accession number 98600;

(g) a polynucleotide comprising the nucleotide sequence of a mature protein coding sequence of clone am856_{—}3 deposited with the ATCC under accession number 98600;

(h) a polynucleotide encoding a mature protein encoded by the cDNA insert of clone am856_{—}3 deposited with the ATCC under accession number 98600;

(i) a polynucleotide encoding a protein comprising the amino acid sequence of SEQ ID NO:22;

(j) a polynucleotide encoding a protein comprising a fragment of the amino acid sequence of SEQ ID NO:22 having biological activity, the fragment comprising eight consecutive amino acids of SEQ ID NO:22;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:21 from nucleotide 469 to nucleotide 609; the nucleotide sequence of SEQ ID NO:21 from nucleotide 574 to nucleotide 609; the nucleotide sequence of SEQ ID NO:21 from nucleotide 214 to nucleotide 369; the nucleotide sequence of the full-length protein coding sequence of clone am856_{—}3 deposited with the ATCC under accession number 98600; or the nucleotide sequence of a mature protein coding sequence of clone am856_{—}3 deposited with the ATCC under accession number 98600. In other preferred embodiments, the polynucleotide encodes the full-length or a mature protein encoded by the cDNA insert of clone am856_{—}3 deposited with the ATCC under accession number 98600. In yet other preferred embodiments, the present invention provides a polynucleotide encoding a protein comprising the amino acid sequence of SEQ ID NO:22 from amino acid 1 to amino acid 38. In further preferred embodiments, the present invention provides a polynucleotide encoding a protein comprising a fragment of the amino acid sequence of SEQ ID NO:22 having biological activity, the fragment preferably comprising eight (more preferably twenty, most preferably thirty) consecutive amino acids of SEQ ID NO:22, or a polynucleotide encoding a protein comprising a fragment of the amino acid sequence of SEQ ID NO:22 having biological activity, the fragment comprising the amino acid sequence from amino acid 18 to amino acid 27 of SEQ ID NO:22.

Other embodiments provide the gene corresponding to the cDNA sequence of SEQ ID NO:21.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:21, but excluding the poly(A) tail at the 3′ end of SEQ ID NO:21; and

(ab) the nucleotide sequence of the cDNA insert of clone am856_{—}3 deposited with the ATCC under accession number 98600; and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:21, but excluding the poly(A) tail at the 3′ end of SEQ ID NO:21; and

(bb) the nucleotide sequence of the cDNA insert of clone am856_{—}3 deposited with the ATCC under accession number 98600; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:21, and extending contiguously from a nucleotide sequence corresponding to the 5′ end of SEQ ID NO:21 to a nucleotide sequence corresponding to the 3′ end of SEQ ID NO:21, but excluding the poly(A) tail at the 3′ end of SEQ ID NO:21. Also preferably the polynucleotide isolated according to the above process comprises a nucleotide sequence corresponding to the cDNA sequence of SEQ ID NO:21 from nucleotide 469 to nucleotide 609, and extending contiguously from a nucleotide sequence corresponding to the 5′ end of said sequence of SEQ ID NO:21 from nucleotide 469 to nucleotide 609, to a nucleotide sequence corresponding to the 3′ end of said sequence of SEQ ID NO:21 from nucleotide 469 to nucleotide 609. Also preferably the polynucleotide isolated according to the above process comprises a nucleotide sequence corresponding to the cDNA sequence of SEQ ID NO:21 from nucleotide 574 to nucleotide 609, and extending contiguously from a nucleotide sequence corresponding to the 5′ end of said sequence of SEQ ID NO:21 from nucleotide 574 to nucleotide 609, to a nucleotide sequence corresponding to the 3′ end of said sequence of SEQ ID NO:21 from nucleotide 574 to nucleotide 609. Also preferably the polynucleotide isolated according to the above process comprises a nucleotide sequence corresponding to the cDNA sequence of SEQ ID NO:21 from nucleotide 214 to nucleotide 369, and extending contiguously from a nucleotide sequence corresponding to the 5′ end of said sequence of SEQ ID NO:21 from nucleotide 214 to nucleotide 369, to a nucleotide sequence corresponding to the 3′ end of said sequence of SEQ ID NO:21 from nucleotide 214 to nucleotide 369.

In other embodiments, the present invention provides a composition comprising a protein, wherein said protein comprises an amino acid sequence selected from the group consisting of:

(a) the amino acid sequence of SEQ ID NO:22;

(b) the amino acid sequence of SEQ ID NO:22 from amino acid 1 to amino acid 38;

(c) fragments of the amino acid sequence of SEQ ID NO:22 comprising eight consecutive amino acids of SEQ ID NO:22; and

(d) the amino acid sequence encoded by the cDNA insert of clone am856_{—}3 deposited with the ATCC under accession number 98600;

the protein being substantially free from other mammalian proteins. Preferably such protein comprises the amino acid sequence of SEQ ID NO:22 or the amino acid sequence of SEQ ID NO:22 from amino acid 1 to amino acid 38. In further preferred embodiments, the present invention provides a protein comprising a fragment of the amino acid sequence of SEQ ID NO:22 having biological activity, the fragment preferably comprising eight (more preferably twenty, most preferably thirty) consecutive amino acids of SEQ ID NO:22, or a protein comprising a fragment of the amino acid sequence of SEQ ID NO:22 having biological activity, the fragment comprising the amino acid sequence from amino acid 18 to amino acid 27 of SEQ ID NO:22.

In one embodiment, the present invention provides a composition comprising an isolated polynucleotide selected from the group consisting of:

(a) a polynucleotide comprising the nucleotide sequence of SEQ ID NO:23;

(b) a polynucleotide comprising the nucleotide sequence of SEQ ID NO:23 from nucleotide 442 to nucleotide 735;

(c) a polynucleotide comprising the nucleotide sequence of SEQ ID NO:23 from nucleotide 520 to nucleotide 735;

(d) a polynucleotide comprising the nucleotide sequence of the full-length protein coding sequence of clone am996_{—}12 deposited with the ATCC under accession number 98600;

(e) a polynucleotide encoding the full-length protein encoded by the cDNA insert of clone am996_{—}12 deposited with the ATCC under accession number 98600;

(f) a polynucleotide comprising the nucleotide sequence of a mature protein coding sequence of clone am996_{—}12 deposited with the ATCC under accession number 98600;

(g) a polynucleotide encoding a mature protein encoded by the cDNA insert of clone am996_{—}12 deposited with the ATCC under accession number 98600;

(h) a polynucleotide encoding a protein comprising the amino acid sequence of SEQ ID NO:24;

(i) a polynucleotide encoding a protein comprising a fragment of the amino acid sequence of SEQ ID NO:24 having biological activity, the fragment comprising eight consecutive amino acids of SEQ ID NO:24;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:23 from nucleotide 442 to nucleotide 735; the nucleotide sequence of SEQ ID NO:23 from nucleotide 520 to nucleotide 735; the nucleotide sequence of the full-length protein coding sequence of clone am996_{—}12 deposited with the ATCC under accession number 98600; or the nucleotide sequence of a mature protein coding sequence of clone am996_{—}12 deposited with the ATCC under accession number 98600. In other preferred embodiments, the polynucleotide encodes the full-length or a mature protein encoded by the cDNA insert of clone am996_{—}12 deposited with the ATCC under accession number 98600. In yet other preferred embodiments, the present invention provides a polynucleotide encoding a protein comprising the amino acid sequence of SEQ ID NO:24 from amino acid 1 to amino acid 90. In further preferred embodiments, the present invention provides a polynucleotide encoding a protein comprising a fragment of the amino acid sequence of SEQ ID NO:24 having biological activity, the fragment preferably comprising eight (more preferably twenty, most preferably thirty) consecutive amino acids of SEQ ID NO:24, or a polynucleotide encoding a protein comprising a fragment of the amino acid sequence of SEQ ID NO:24 having biological activity, the fragment comprising the amino acid sequence from amino acid 44 to amino acid 53 of SEQ ID NO:24.

Other embodiments provide the gene corresponding to the cDNA sequence of SEQ ID NO:23.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:23, but excluding the poly(A) tail at the 3′ end of SEQ ID NO:23; and

(ab) the nucleotide sequence of the cDNA insert of clone am996_{—}12 deposited with the ATCC under accession number 98600;and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:23, but excluding the poly(A) tail at the 3′ end of SEQ ID NO:23; and

(bb) the nucleotide sequence of the cDNA insert of clone am996_{—}12 deposited with the ATCC under accession number 98600; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:23, and extending contiguously from a nucleotide sequence corresponding to the 5′ end of SEQ ID NO:23 to a nucleotide sequence corresponding to the 3′ end of SEQ ID NO:23 , but excluding the poly(A) tail at the 3′ end of SEQ ID NO:23. Also preferably the polynucleotide isolated according to the above process comprises a nucleotide sequence corresponding to the cDNA sequence of SEQ ID NO:23 from nucleotide 442 to nucleotide 735, and extending contiguously from a nucleotide sequence corresponding to the 5′ end of said sequence of SEQ ID NO:23 from nucleotide 442 to nucleotide 735, to a nucleotide sequence corresponding to the 3′ end of said sequence of SEQ ID NO:23 from nucleotide 442 to nucleotide 735. Also preferably the polynucleotide isolated according to the above process comprises a nucleotide sequence corresponding to the cDNA sequence of SEQ ID NO:23 from nucleotide 520 to nucleotide 735, and extending contiguously from a nucleotide sequence corresponding to the 5′ end of said sequence of SEQ ID NO:23 from nucleotide 520 to nucleotide 735, to a nucleotide sequence corresponding to the 3′ end of said sequence of SEQ ID NO:23 from nucleotide 520 to nucleotide 735.

In other embodiments, the present invention provides a composition comprising a protein, wherein said protein comprises an amino acid sequence selected from the group consisting of:

(a) the amino acid sequence of SEQ ID NO:24;

(b) the amino acid sequence of SEQ ID NO:24 from amino acid 1 to amino acid 90;

(c) fragments of the amino acid sequence of SEQ ID NO:24 comprising eight consecutive amino acids of SEQ ID NO:24; and

(d) the amino acid sequence encoded by the cDNA insert of clone am996_{—}12 deposited with the ATCC under accession number 98600;

the protein being substantially free from other mammalian proteins. Preferably such protein comprises the amino acid sequence of SEQ ID NO:24 or the amino acid sequence of SEQ ID NO:24 from amino acid 1 to amino acid 90. In further preferred embodiments, the present invention provides a protein comprising a fragment of the amino acid sequence of SEQ ID NO:24 having biological activity, the fragment preferably comprising eight (more preferably twenty, most preferably thirty) consecutive amino acids of SEQ ID NO:24, or a protein comprising a fragment of the amino acid sequence of SEQ ID NO:24 having biological activity, the fragment comprising the amino acid sequence from amino acid 44 to amino acid 53 of SEQ ID NO:24.

In one embodiment, the present invention provides a composition comprising an isolated polynucleotide selected from the group consisting of:

(a) a polynucleotide comprising the nucleotide sequence of SEQ ID NO:25;

(b) a polynucleotide comprising the nucleotide sequence of SEQ ID NO:25 from nucleotide 127 to nucleotide 240;

(c) a polynucleotide comprising the nucleotide sequence of the full-length protein coding sequence of clone cc69_{—}1 deposited with the ATCC under accession number 98600;

(d) a polynucleotide encoding the full-length protein encoded by the cDNA insert of clone cc69_{—}1 deposited with the ATCC under accession number 98600;

(e) a polynucleotide comprising the nucleotide sequence of a mature protein coding sequence of clone cc69_{—}1 deposited with the ATCC under accession number 98600;

(f) a polynucleotide encoding a mature protein encoded by the cDNA insert of clone cc69_{—}1 deposited with the ATCC under accession number 98600;

(g) a polynucleotide encoding a protein comprising the amino acid sequence of SEQ ID NO:26;

(h) a polynucleotide encoding a protein comprising a fragment of the amino acid sequence of SEQ ID NO:26 having biological activity, the fragment comprising eight consecutive amino acids of SEQ ID NO:26;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:25 from nucleotide 127 to nucleotide 240; the nucleotide sequence of the full-length protein coding sequence of clone cc69_{—}1 deposited with the ATCC under accession number 98600; or the nucleotide sequence of a mature protein coding sequence of clone cc69_{—}1 deposited with the ATCC under accession number 98600. In other preferred embodiments, the polynucleotide encodes the full-length or a mature protein encoded by the cDNA insert of clone cc69_{—}1 deposited with the ATCC under accession number 98600.

In further preferred embodiments, the present invention provides a polynucleotide encoding a protein comprising a fragment of the amino acid sequence of SEQ ID NO:26 having biological activity, the fragment preferably comprising eight (more preferably twenty, most preferably thirty) consecutive amino acids of SEQ ID NO:26, or a polynucleotide encoding a protein comprising a fragment of the amino acid sequence of SEQ ID NO:26 having biological activity, the fragment comprising the amino acid sequence from amino acid 14 to amino acid 23 of SEQ ID NO:26.

Other embodiments provide the gene corresponding to the cDNA sequence of SEQ ID NO:25.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:25, but excluding the poly(A) tail at the 3′ end of SEQ ID NO:25; and

(ab) the nucleotide sequence of the cDNA insert of clone cc69_{—}1 deposited with the ATCC under accession number 98600; and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:25, but excluding the poly(A) tail at the 3′ end of SEQ ID NO:25; and

(bb) the nucleotide sequence of the cDNA insert of clone cc69_{—}1 deposited with the ATCC under accession number 98600; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:25, and extending contiguously from a nucleotide sequence corresponding to the 5′ end of SEQ ID NO:25 to a nucleotide sequence corresponding to the 3′ end of SEQ ID NO:25, but excluding the poly(A) tail at the 3′ end of SEQ ID NO:25. Also preferably the polynucleotide isolated according to the above process comprises a nucleotide sequence corresponding to the cDNA sequence of SEQ ID NO:25 from nucleotide 127 to nucleotide 240, and extending contiguously from a nucleotide sequence corresponding to the 5′ end of said sequence of SEQ ID NO:25 from nucleotide 127 to nucleotide 240, to a nucleotide sequence corresponding to the 3′ end of said sequence of SEQ ID NO:25 from nucleotide 127 to nucleotide 240.

In other embodiments, the present invention provides a composition comprising a protein, wherein said protein comprises an amino acid sequence selected from the group consisting of:

(a) the amino acid sequence of SEQ ID NO:26;

(b) fragments of the amino acid sequence of SEQ ID NO:26 comprising eight consecutive amino acids of SEQ ID NO:26; and

(c) the amino acid sequence encoded by the cDNA insert of clone cc69_{—}1 deposited with the ATCC under accession number 98600;

the protein being substantially free from other mammalian proteins. Preferably such protein comprises the amino acid sequence of SEQ ID NO:26. In further preferred embodiments, the present invention provides a protein comprising a fragment of the amino acid sequence of SEQ ID NO:26 having biological activity, the fragment preferably comprising eight (more preferably twenty, most preferably thirty) consecutive amino acids of SEQ ID NO:26, or a protein comprising a fragment of the amino acid sequence of SEQ ID NO:26 having biological activity, the fragment comprising the amino acid sequence from amino acid 14 to amino acid 23 of SEQ ID NO:26.

In one embodiment, the present invention provides a composition comprising an isolated polynucleotide selected from the group consisting of:

(a) a polynucleotide comprising the nucleotide sequence of SEQ ID NO:27;

(b) a polynucleotide comprising the nucleotide sequence of SEQ ID NO:27 from nucleotide 156 to nucleotide 413;

(c) a polynucleotide comprising the nucleotide sequence of SEQ ID NO:27 from nucleotide 198 to nucleotide 413;

(d) a polynucleotide comprising the nucleotide sequence of the full-length protein coding sequence of clone cc162_{—}1 deposited with the ATCC under accession number 98600;

(e) a polynucleotide encoding the full-length protein encoded by the cDNA insert of clone cc162_{—}1 deposited with the ATCC under accession number 98600;

(f) a polynucleotide comprising the nucleotide sequence of a mature protein coding sequence of clone cc162_{—}1 deposited with the ATCC under accession number 98600;

(g) a polynucleotide encoding a mature protein encoded by the cDNA insert of clone cc162_{—}1 deposited with the ATCC under accession number 98600;

(h) a polynucleotide encoding a protein comprising the amino acid sequence of SEQ ID NO:28;

(i) a polynucleotide encoding a protein comprising a fragment of the amino acid sequence of SEQ ID NO:28 having biological activity, the fragment comprising eight consecutive amino acids of SEQ ID NO:28;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:27 from nucleotide 156 to nucleotide 413; the nucleotide sequence of SEQ ID NO:27 from nucleotide 198 to nucleotide 413; the nucleotide sequence of the full-length protein coding sequence of clone cc162_{—}1 deposited with the ATCC under accession number 98600; or the nucleotide sequence of a mature protein coding sequence of clone cc162_{—}1 deposited with the ATCC under accession number 98600. In other preferred embodiments, the polynucleotide encodes the full-length or a mature protein encoded by the cDNA insert of clone cc162_{—}1 deposited with the ATCC under accession number 98600. In yet other preferred embodiments, the present invention provides a polynucleotide encoding a protein comprising the amino acid sequence of SEQ ID NO:28 from amino acid 1 to amino acid 66. In further preferred embodiments, the present invention provides a polynucleotide encoding a protein comprising a fragment of the amino acid sequence of SEQ ID NO:28 having biological activity, the fragment preferably comprising eight (more preferably twenty, most preferably thirty) consecutive amino acids of SEQ ID NO:28, or a polynucleotide encoding a protein comprising a fragment of the amino acid sequence of SEQ ID NO:28 having biological activity, the fragment comprising the amino acid sequence from amino acid 38 to amino acid 47 of SEQ ID NO:28.

Other embodiments provide the gene corresponding to the cDNA sequence of SEQ ID NO:27.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:27, but excluding the poly(A) tail at the 3′ end of SEQ ID NO:27; and

(ab) the nucleotide sequence of the cDNA insert of clone cc162_{—}1 deposited with the ATCC under accession number 98600; and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:27, but excluding the poly(A) tail at the 3′ end of SEQ ID NO:27; and

(bb) the nucleotide sequence of the cDNA insert of clone cc162_{—}1 deposited with the ATCC under accession number 98600; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:27, and extending contiguously from a nucleotide sequence corresponding to the 5′ end of SEQ ID NO:27 to a nucleotide sequence corresponding to the 3′ end of SEQ ID NO:27, but excluding the poly(A) tail at the 3′ end of SEQ ID NO:27. Also preferably the polynucleotide isolated according to the above process comprises a nucleotide sequence corresponding to the cDNA sequence of SEQ ID NO:27 from nucleotide 156 to nucleotide 413, and extending contiguously from a nucleotide sequence corresponding to the 5′ end of said sequence of SEQ ID NO:27 from nucleotide 156 to nucleotide 413, to a nucleotide sequence corresponding to the 3′ end of said sequence of SEQ ID NO:27 from nucleotide 156 to nucleotide 413. Also preferably the polynucleotide isolated according to the above process comprises a nucleotide sequence corresponding to the cDNA sequence of SEQ ID NO:27 from nucleotide 198 to nucleotide 413, and extending contiguously from a nucleotide sequence corresponding to the 5′ end of said sequence of SEQ ID NO:27 from nucleotide 198 to nucleotide 413, to a nucleotide sequence corresponding to the 3′ end of said sequence of SEQ ID NO:27 from nucleotide 198 to nucleotide 413.

In other embodiments, the present invention provides a composition comprising a protein, wherein said protein comprises an amino acid sequence selected from the group consisting of:

(a) the amino acid sequence of SEQ ID NO:28;

(b) the amino acid sequence of SEQ ID NO:28 from amino acid 1 to amino acid 66;

(c) fragments of the amino acid sequence of SEQ ID NO:28 comprising eight consecutive amino acids of SEQ ID NO:28; and

(d) the amino acid sequence encoded by the cDNA insert of clone cc162_{—}1 deposited with the ATCC under accession number 98600;

the protein being substantially free from other manmalian proteins. Preferably such protein comprises the amino acid sequence of SEQ ID NO:28 or the amino acid sequence of SEQ ID NO:28 from amino acid 1 to amino acid 66. In further preferred embodiments, the present invention provides a protein comprising a fragment of the amino acid sequence of SEQ ID NO:28 having biological activity, the fragment preferably comprising eight (more preferably twenty, most preferably thirty) consecutive amino acids of SEQ ID NO:28, or a protein comprising a fragment of the amino acid sequence of SEQ ID NO:28 having biological activity, the fragment comprising the amino acid sequence from amino acid 38 to amino acid 47 of SEQ ID NO:28.

In one embodiment, the present invention provides a composition comprising an isolated polynucleotide selected from the group consisting of:

(a) a polynucleotide comprising the nucleotide sequence of SEQ ID NO:29;

(b) a polynucleotide comprising the nucleotide sequence of SEQ ID NO:29 from nucleotide 180 to nucleotide 737;

(c) a polynucleotide comprising the nucleotide sequence of SEQ ID NO:29 from nucleotide 240 to nucleotide 737;

(d) a polynucleotide comprising the nucleotide sequence of the full-length protein coding sequence of clone if87_{—}1 deposited with the ATCC under accession number 98600;

(e) a polynucleotide encoding the full-length protein encoded by the cDNA insert of clone if87_{—}1 deposited with the ATCC under accession number 98600;

(f) a polynucleotide comprising the nucleotide sequence of a mature protein coding sequence of clone if87_{—}1 deposited with the ATCC under accession number 98600;

(g) a polynucleotide encoding a mature protein encoded by the cDNA insert of clone if87_{—}1 deposited with the ATCC under accession number 98600;

(h) a polynucleotide encoding a protein comprising the amino acid sequence of SEQ ID NO:30;

(i) a polynucleotide encoding a protein comprising a fragment of the amino acid sequence of SEQ ID NO:30 having biological activity, the fragment comprising eight consecutive amino acids of SEQ ID NO:30;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:29 from nucleotide 180 to nucleotide 737; the nucleotide sequence of SEQ ID NO:29 from nucleotide 240 to nucleotide 737; the nucleotide sequence of the full-length protein coding sequence of clone if87_{—}1 deposited with the ATCC under accession number 98600; or the nucleotide sequence of a mature protein coding sequence of clone if87_{—}1 deposited with the ATCC under accession number 98600. In other preferred embodiments, the polynucleotide encodes the full-length or a mature protein encoded by the cDNA insert of clone if87_{—}1 deposited with the ATCC under accession number 98600. In yet other preferred embodiments, the present invention provides a polynucleotide encoding a protein comprising the amino acid sequence of SEQ ID NO:30 from amino acid 1 to amino acid 88. In further preferred embodiments, the present invention provides a polynucleotide encoding a protein comprising a fragment of the amino acid sequence of SEQ ID NO:30 having biological activity, the fragment preferably comprising eight (more preferably twenty, most preferably thirty) consecutive amino acids of SEQ ID NO:30, or a polynucleotide encoding a protein comprising a fragment of the amnino acid sequence of SEQ ID NO:30 having biological activity, the fragment comprising the amino acid sequence from amino acid 88 to amino acid 97 of SEQ ID NO:30.

Other embodiments provide the gene corresponding to the cDNA sequence of SEQ ID NO:29.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:29, but excluding the poly(A) tail at the 3′ end of SEQ ID NO:29; and

(ab) the nucleotide sequence of the cDNA insert of clone if87_{—}1 deposited with the ATCC under accession number 98600; and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:29, but excluding the poly(A) tail at the 3′ end of SEQ ID NO:29; and

(bb) the nucleotide sequence of the cDNA insert of clone if87_{—}1 deposited with the ATCC under accession number 98600; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:29, and extending contiguously from a nudeotide sequence corresponding to the 5′ end of SEQ ID NO:29 to a nucleotide sequence corresponding to the 3′ end of SEQ ID NO:29, but excluding the poly(A) tail at the 3′ end of SEQ ID NO:29. Also preferably the polynucleotide isolated according to the above process comprises a nucleotide sequence corresponding to the cDNA sequence of SEQ ID NO:29 from nucleotide 180 to nucleotide 737, and extending contiguously from a nucleotide sequence corresponding to the 5′ end of said sequence of SEQ ID NO:29 from nucleotide 180 to nucleotide 737, to a nucleotide sequence corresponding to the 3′ end of said sequence of SEQ ID NO:29 from nucleotide 180 to nucleotide 737. Also preferably the polynucleotide isolated according to the above process comprises a nucleotide sequence corresponding to the cDNA sequence of SEQ ID NO:29 from nucleotide 240 to nucleotide 737, and extending contiguously from a nucleotide sequence corresponding to the 5′ end of said sequence of SEQ ID NO:29 from nucleotide 240 to nucleotide 737, to a nucleotide sequence corresponding to the 3′ end of said sequence of SEQ ID NO:29 from nucleotide 240 to nucleotide 737.

In other embodiments, the present invention provides a composition comprising a protein, wherein said protein comprises an amino acid sequence selected from the group consisting of:

(a) the amino acid sequence of SEQ ID NO:30;

(b) the amino acid sequence of SEQ ID NO:30 from amino acid 1 to amino acid 88;

(c) fragments of the amino acid sequence of SEQ ID NO:30 comprising eight consecutive amino acids of SEQ ID NO:30; and

(d) the amino acid sequence encoded by the cDNA insert of clone if87_{—}1 deposited with the ATCC under accession number 98600;

the protein being substantially free from other mammalian proteins. Preferably such protein comprises the amino acid sequence of SEQ ID NO:30 or the amino acid sequence of SEQ ID NO:30 from amino acid 1 to amino acid 88. In further preferred embodiments, the present invention provides a protein comprising a fragment of the amino acid sequence of SEQ ID NO:30 having biological activity, the fragment preferably comprising eight (more preferably twenty, most preferably thirty) consecutive amino acids of SEQ ID NO:30, or a protein comprising a fragment of the amino acid sequence of SEQ ID NO:30 having biological activity, the fragment comprising the amino acid sequence from amino acid 88 to amino acid 97 of SEQ ID NO:30.

In one embodiment, the present invention provides a composition comprising an isolated polynucleotide selected from the group consisting of:

(a) a polynucleotide comprising the nucleotide sequence of SEQ ID NO:31;

(b) a polynucleotide comprising the nucleotide sequence of SEQ ID NO:31 from nucleotide 2294 to nucleotide 2845;

(c) a polynucleotide comprising the nucleotide sequence of SEQ ID NO:31 from nucleotide 2387 to nucleotide 2845;

(d) a polynucleotide comprising the nucleotide sequence of the full-length protein coding sequence of clone nn103_{—}4 deposited with the ATCC under accession number 98600;

(e) a polynucleotide encoding the full-length protein encoded by the cDNA insert of clone nn103_{—}4 deposited with the ATCC under accession number 98600;

(f) a polynucleotide comprising the nucleotide sequence of a mature protein coding sequence of clone nn103_{—}4 deposited with the ATCC under accession number 98600;

(g) a polynucleotide encoding a mature protein encoded by the cDNA insert of clone nn103_{—}4 deposited with the ATCC under accession number 98600;

(h) a polynucleotide encoding a protein comprising the amino acid sequence of SEQ ID NO:32;

(i) a polynucleotide encoding a protein comprising a fragment of the amino acid sequence of SEQ ID NO:32 having biological activity, the fragment comprising eight consecutive amino acids of SEQ ID NO:32;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:31 from nucleotide 2294 to nucleotide 2845; the nucleotide sequence of SEQ ID NO:31 from nucleotide 2387 to nucleotide 2845; the nucleotide sequence of the full-length protein coding sequence of clone nn103_{—}4 deposited with the ATCC under accession number 98600; or the nucleotide sequence of a mature protein coding sequence of clone nn103_{—}4 deposited with the ATCC under accession number 98600. In other preferred embodiments, the polynucleotide encodes the full-length or a mature protein encoded by the cDNA insert of clone nn103_{—}4 deposited with the ATCC under accession number 98600. In yet other preferred embodiments, the present invention provides a polynucleotide encoding a protein comprising the amino acid sequence of SEQ ID NO:32 from amino acid 12 to amino acid 137. In further preferred embodiments, the present invention provides a polynucleotide encoding a protein comprising a fragment of the amino acid sequence of SEQ ID NO:32 having biological activity, the fragment preferably comprising eight (more preferably twenty, most preferably thirty) consecutive amino acids of SEQ ID NO:32, or a polynucleotide encoding a protein comprising a fragment of the amino acid sequence of SEQ ID NO:32 having biological activity, the fragment comprising the amino acid sequence from amino acid 87 to amino acid 96 of SEQ ID NO:32.

Other embodiments provide the gene corresponding to the cDNA sequence of SEQ ID NO:31.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:31, but excluding the poly(A) tail at the 3′ end of SEQ ID NO:31; and

(ab) the nucleotide sequence of the cDNA insert of clone nn103_{—}4 deposited with the ATCC under accession number 98600; and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:31, but excluding the poly(A) tail at the 3′ end of SEQ ID NO:31; and

(bb) the nucleotide sequence of the cDNA insert of clone nn103_{—}4 deposited with the ATCC under accession number 98600; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:31, and extending contiguously from a nucleotide sequence corresponding to the 5′ end of SEQ ID NO:31 to a nucleotide sequence corresponding to the 3′ end of SEQ ID NO:31 , but excluding the poly(A) tail at the 3′ end of SEQ ID NO:31. Also preferably the polynucleotide isolated according to the above process comprises a nucleotide sequence corresponding to the cDNA sequence of SEQ ID NO:31 from nucleotide 2294 to nucleotide 2845, and extending contiguously from a nucleotide sequence corresponding to the 5′ end of said sequence of SEQ ID NO:31 from nucleotide 2294 to nucleotide 2845, to a nucleotide sequence corresponding to the 3′ end of said sequence of SEQ ID NO:31 from nucleotide 2294 to nucleotide 2845. Also preferably the polynucleotide isolated according to the above process comprises a nucleotide sequence corresponding to the cDNA sequence of SEQ ID NO:31 from nucleotide 2387 to nucleotide 2845, and extending contiguously from a nucleotide sequence corresponding to the 5′ end of said sequence of SEQ ID NO:31 from nucleotide 2387 to nucleotide 2845, to a nucleotide sequence corresponding to the 3′ end of said sequence of SEQ ID NO:31 from nucleotide 2387 to nucleotide 2845.

In other embodiments, the present invention provides a composition comprising a protein, wherein said protein comprises an amino acid sequence selected from the group consisting of:

(a) the amino acid sequence of SEQ ID NO:32;

(b) the amino acid sequence of SEQ ID NO:32 from amino acid 12 to amino acid 137;

(c) fragments of the amino acid sequence of SEQ ID NO:32 comprising eight consecutive amino acids of SEQ ID NO:32; and

(d) the amino acid sequence encoded by the cDNA insert of clone nn103_{—}4 deposited with the ATCC under accession number 98600;

the protein being substantially free from other mammalian proteins. Preferably such protein comprises the amino acid sequence of SEQ ID NO:32 or the amino acid sequence of SEQ ID NO:32 from amino acid 12 to amino acid 137. In further preferred embodiments, the present invention provides a protein comprising a fragment of the amino acid sequence of SEQ ID NO:32 having biological activity, the fragment preferably comprising eight (more preferably twenty, most preferably thirty) consecutive amino acids of SEQ ID NO:32, or a protein comprising a fragment of the amino acid sequence of SEQ ID NO:32 having biological activity, the fragment comprising the amino acid sequence from amino acid 87 to amino acid 96 of SEQ ID NO:32.

In one embodiment, the present invention provides a composition comprising an isolated polynucleotide selected from the group consisting of:

(a) a polvnucleotide comprising the nucleotide sequence of SEQ ID NO:33;

(b) a polynucleotide comprising the nucleotide sequence of SEQ ID NO:33 from nucleotide 1280 to nucleotide 1504;

(c) a polynucleotide comprising the nucleotide sequence of the full-length protein coding sequence of clone np206_{—}8 deposited with the ATCC under accession number 98600;

(d) a polynucleotide encoding the full-length protein encoded by the cDNA insert of clone np206_{—}8 deposited with the ATCC under accession number 98600;

(e) a polynucleotide comprising the nucleotide sequence of a mature protein coding sequence of clone np206_{—}8 deposited with the ATCC under accession number 98600;

(f) a polynucleotide encoding a mature protein encoded by the cDNA insert of clone np206_{—}8 deposited with the ATCC under accession number 98600;

(g) a polynucleotide encoding a protein comprising the amino acid sequence of SEQ ID NO:34;

(h) a polynucleotide encoding a protein comprising a fragment of the amino acid sequence of SEQ ID NO:34 having biological activity, the fragment comprising eight consecutive amino acids of SEQ ID NO:34;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:33 from nucleotide 1280 to nucleotide 1504; the nucleotide sequence of the full-length protein coding sequence of clone np206_{—}8 deposited with the ATCC under accession number 98600; or the nucleotide sequence of a mature protein coding sequence of clone np206_{—}8 deposited with the ATCC under accession number 98600. In other preferred embodiments, the polynucleotide encodes the full-length or a mature protein encoded by the cDNA insert of clone np206_{—}8 deposited with the ATCC under accession number 98600. In yet other preferred embodiments, the present invention provides a polynucleotide encoding a protein comprising the amino acid sequence of SEQ ID NO:34 from amino acid 1 to amino acid 26. In further preferred embodiments, the present invention provides a polynucleotide encoding a protein comprising a fragment of the amino acid sequence of SEQ ID NO:34 having biological activity, the fragment preferably comprising eight (more preferably twenty, most preferably thirty) consecutive amino acids of SEQ ID NO:34, or a polynucleotide encoding a protein comprising a fragment of the amino acid sequence of SEQ ID NO:34 having biological activity, the fragment comprising the amino acid sequence from amino acid 32 to amino acid 41 of SEQ ID NO:34.

Other embodiments provide the gene corresponding to the cDNA sequence of SEQ ID NO:33.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:33, but excluding the poly(A) tail at the 3′ end of SEQ ID NO:33; and

(ab) the nucleotide sequence of the cDNA insert of clone np206_{—8} deposited with the ATCC under accession number 98600; and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:33, but excluding the poly(A) tail at the 3′ end of SEQ ID NO:33; and

(bb) the nucleotide sequence of the cDNA insert of clone np206_{—}8 deposited with the ATCC under accession number 98600; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:33, and extending contiguously from a nucleotide sequence corresponding to the 5′ end of SEQ ID NO:33 to a nucleotide sequence corresponding to the 3′ end of SEQ ID NO:33, but excluding the poly(A) tail at the 3′ end of SEQ ID NO:33. Also preferably the polynucleotide isolated according to the above process comprises a nucleotide sequence corresponding to the cDN=A sequence of SEQ ID NO:33 from nucleotide 1280 to nucleotide 1504, and extending contiguously from a nucleotide sequence corresponding to the 5′ end of said sequence of SEQ ID NO:33 from nucleotide 1280 to nucleotide 1504, to a nucleotide sequence corresponding to the 3′ end of said sequence of SEQ ID NO:33 from nucleotide 1280 to nucleotide 1504.

In other embodiments, the present invention provides a composition comprising a protein, wherein said protein comprises an amino acid sequence selected from the group consisting of:

(a) the amino acid sequence of SEQ ID NO:34;

(b) the amino acid sequence of SEQ ID NO:34 from amino acid 1 to amino acid 26;

(c) fragments of the amino acid sequence of SEQ ID NO:34 comprising eight consecutive amino acids of SEQ ID NO:34; and

(d) the amino acid sequence encoded by the cDNA insert of clone np206_{—}8 deposited with the ATCC under accession number 98600;

the protein being substantially free from other mammalian proteins. Preferably such a protein comprises the amino acid sequence of SEQ ID NO:34 or the amino acid sequence of SEQ ID NO:34 from amino acid 1 to amino acid 26. In further preferred embodiments, the present invention provides a protein comprising a fragment of the amino acid sequence of SEQ ID NO:34 having biological activity, the fragment preferably comprising eight (more preferably twenty, most preferably thirty) consecutive amino acids of SEQ ID NO:34, or a protein comprising a fragment of the amino acid sequence of SEQ ID NO:34 having biological activity, the fragment comprising the amino acid sequence from amino acid 32 to amino acid 41 of SEQ ID NO:34.

In one embodiment, the present invention provides a composition comprising an isolated polynucleotide selected from the group consisting of:

(a) a polynucleotide comprising the nucleotide sequence of SEQ ID NO:35;

(b) a polynucleotide comprising the nucleotide sequence of SEQ ID NO:35 from nucleotide 133 to nucleotide 432;

(c) a polynucleotide comprising the nucleotide sequence of the full-length protein coding sequence of clone nt746_{—}4 deposited with the ATCC under accession number 98600;

(d) a polynucleotide encoding the full-length protein encoded by the cDNA insert of clone nt746_{—}4 deposited with the ATCC under accession number 98600;

(e) a polynucleotide comprising the nucleotide sequence of a mature protein coding sequence of clone nt746_{—}4 deposited with the ATCC under accession number 98600;

(f) a polynucleotide encoding a mature protein encoded by the cDNA insert of clone nt746_{—}4 deposited with the ATCC under accession number 98600;

(g) a polynucleotide encoding a protein comprising the amino acid sequence of SEQ ID NO:36;

(h) a polynucleotide encoding a protein comprising a fragment of the amino acid sequence of SEQ ID NO:36 having biological activity, the fragment comprising eight consecutive amino acids of SEQ ID NO:36;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:35 from nucleotide 133 to nucleotide 432; the nucleotide sequence of the full-length protein coding sequence of clone nt746_{—}4 deposited with the ATCC under accession number 98600; or the nucleotide sequence of a mature protein coding sequence of clone nt746_{—}4 deposited with the ATCC under accession number 98600. In other preferred embodiments, the polynucleotide encodes the full-length or a mature protein encoded by the cDNA insert of clone nt746_{—}4 deposited with the ATCC under accession number 98600. In yet other preferred embodiments, the present invention provides a polynucleotide encoding a protein comprising the amino acid sequence of SEQ ID NO:36 from amino acid 1 to amino acid 70. In further preferred embodiments, the present invention provides a polynucleotide encoding a protein comprising a fragment of the amino acid sequence of SEQ ID NO:36 having biological activity, the fragment preferably comprising eight (more preferably twenty, most preferably thirty) consecutive amino acids of SEQ ID NO:36, or a polynucleotide encoding a protein comprising a fragment of the amino acid sequence of SEQ ID NO:36 having biological activity, the fragment comprising the amino acid sequence from amino acid 45 to amino acid 54 of SEQ ID NO:36.

Other embodiments provide the gene corresponding to the cDNA sequence of SEQ ID NO:35.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:35, but excluding the poly(A) tail at the 3′ end of SEQ ID NO:35; and

(ab) the nucleotide sequence of the cDNA insert of clone nt746_{—}4 deposited with the ATCC under accession number 98600; and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:35, but excluding the poly(A) tail at the 3′ end of SEQ ID NO:35; and

(bb) the nucleotide sequence of the cDNA insert of clone nt746_{—}4 deposited with the ATCC under accession number 98600; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:35, and extending contiguously from a nucleotide sequence corresponding to the 5′ end of SEQ ID NO:35 to a nucleotide sequence corresponding to the 3′ end of SEQ ID NO:35, but excluding the poly(A) tail at the 3′ end of SEQ ID NO:35. Also preferably the polynucleotide isolated according to the above process comprises a nucleotide sequence corresponding to the cDNA sequence of SEQ ID NO:35 from nucleotide 133 to nucleotide 432, and extending contiguously from a nucleotide sequence corresponding to the 5′ end of said sequence of SEQ ID NO:35 from nucleotide 133 to nucleotide 432, to a nucleotide sequence corresponding to the 3′ end of said sequence of SEQ ID NO:35 from nucleotide 133 to nucleotide 432.

In other embodiments, the present invention provides a composition comprising a protein, wherein said protein comprises an amino acid sequence selected from the group consisting of:

(a) the amino acid sequence of SEQ ID NO:36;

(b) the amino acid sequence of SEQ ID NO:36 from amino acid 1 to amino acid 70;

(c) fragments of the amino acid sequence of SEQ ID NO:36 comprising eight consecutive amino acids of SEQ ID NO:36; and

(d) the amino acid sequence encoded by the cDNA insert of clone nt746_{—}4 deposited with the ATCC under accession number 98600;

the protein being substantially free from other mammalian proteins. Preferably such protein comprises the amino acid sequence of SEQ ID NO:36 or the amino acid sequence of SEQ ID NO:36 from amino acid 1 to amino acid 70. In further preferred embodiments, the present invention provides a protein comprising a fragment of the amino acid sequence of SEQ ID NO:36 having biological activity, the fragment preferably comprising eight (more preferably twenty, most preferably thirty) consecutive amino acids of SEQ ID NO:36, or a protein comprising a fragment of the amino acid sequence of SEQ ID NO:36 having biological activity, the fragment comprising the amino acid sequence from amino acid 45 to amino acid 54 of SEQ ID NO:36.

In one embodiment, the present invention provides a composition comprising an isolated polynucleotide selected from the group consisting of:

(a) a polynucleotide comprising the nucleotide sequence of SEQ ID NO:37;

(b) a polynucleotide comprising the nucleotide sequence of SEQ ID NO:37 from nucleotide 31 to nucleotide 201;

(c) a polynucleotide comprising the nucleotide sequence of the full-length protein coding sequence of clone pe286_{—}1 deposited with the ATCC under accession number 98600;

(d) a polynucleotide encoding the full-length protein encoded by the cDNA insert of clone pe286_{—}1 deposited with the ATCC under accession number 98600;

(e) a polynucleotide comprising the nucleotide sequence of a mature protein coding sequence of clone pe286_{—}1 deposited with the ATCC under accession number 98600;

(f) a polynucleotide encoding a mature protein encoded by the cDNA insert of clone pe286_{—}1 deposited with the ATCC under accession number 98600;

(g) a polynucleotide encoding a protein comprising the amino acid sequence of SEQ ID NO:38;

(h) a polynucleotide encoding a protein comprising a fragment of the amino acid sequence of SEQ ID NO:38 having biological activity, the fragment comprising eight consecutive amino acids of SEQ ID NO:38;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:37 from nucleotide 31 to nucleotide 201; the nucleotide sequence of the full-length protein coding sequence of clone pe286_{—}1 deposited with the ATCC under accession number 98600; or the nucleotide sequence of a mature protein coding sequence of clone pe286_{—}1 deposited with the ATCC under accession number 98600. In other preferred embodiments, the polynucleotide encodes the full-length or a mature protein encoded by the cDNA insert of clone pe286_{—}1 deposited with the ATCC under accession number 98600. In yet other preferred embodiments, the present invention provides a polynucleotide encoding a protein comprising the amino acid sequence of SEQ ID NO:38 from amino acid 1 to amino acid 49. In further preferred embodiments, the present invention provides a polynucleotide encoding a protein comprising a fragment of the amino acid sequence of SEQ ID NO:38 having biological activity, the fragment preferably comprising eight (more preferably twenty, most preferably thirty) consecutive amino acids of SEQ ID NO:38, or a polynucleotide encoding a protein comprising a fragment of the amino acid sequence of SEQ ID NO:38 having biological activity, the fragment comprising the amino acid sequence from amino acid 23 to amino acid 32 of SEQ ID NO:38.

Other embodiments provide the gene corresponding to the cDNA sequence of SEQ ID NO:37.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:37, but excluding the poly(A) tail at the 3′ end of SEQ ID NO:37; and

(ab) the nucleotide sequence of the cDNA insert of clone pe286_{—}1 deposited with the ATCC under accession number 98600; and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:37, but excluding the poly(A) tail at the 3′ end of SEQ ID NO:37; and

(bb) the nucleotide sequence of the cDNA insert of clone pe286_{—}1 deposited with the ATCC under accession number 98600; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:37, and extending contiguously from a nucleotide sequence corresponding to the 5′ end of SEQ ID NO:37 to a-nucleotide sequence corresponding to the 3′ end of SEQ ID NO:37, but excluding the poly(A) tail at the 3′ end of SEQ ID NO:37. Also preferably the polynucleotide isolated according to the above process comprises a nucleotide sequence corresponding to the cDNA sequence of SEQ ID NO:37 from nucleotide 31 to nucleotide 201, and extending contiguously from a nucleotide sequence corresponding to the 5′ end of said sequence of SEQ ID NO:37 from nucleotide 31 to nucleotide 201, to a nucleotide sequence corresponding to the 3′ end of said sequence of SEQ ID NO:37 from nucleotide 31 to nucleotide 201.

In other embodiments, the present invention provides a composition comprising a protein, wherein said protein comprises an amino acid sequence selected from the group consisting of:

(a) the amino acid sequence of SEQ ID NO:38;

(b) the amino acid sequence of SEQ ID NO:38 from amino acid 1 to amino acid 49;

(c) fragments of the amino acid sequence of SEQ ID NO:38 comprising eight consecutive amino acids of SEQ ID NO:38; and

(d) the amino acid sequence encoded by the cDNA insert of clone pe286_{—}1 deposited with the ATCC under accession number 98600;

the protein being substantially free from other mammalian proteins. Preferably such protein comprises the amino acid sequence of SEQ ID NO:38 or the amino acid sequence of SEQ ID NO:38 from amino acid 1 to amino acid 49. In further preferred embodiments, the present invention provides a protein comprising a fragment of the amino acid sequence of SEQ ID NO:38 having biological activity, the fragment preferably comprising eight (more preferably twenty, most preferably thirty) consecutive amino acids of SEQ ID NO:38, or a protein comprising a fragment of the amino acid sequence of SEQ ID NO:38 having biological activity, the fragment comprising the amino acid sequence from amino acid 23 to amino acid 32 of SEQ ID NO:38.

In one embodiment, the present invention provides a composition comprising an isolated polynucleotide selected from the group consisting of:

(a) a polynucleotide comprising the nucleotide sequence of SEQ ID NO:39;

(b) a polynucleotide comprising the nucleotide sequence of SEQ ID NO:39 from nucleotide 843 to nucleotide 1004;

(c) a polynucleotide comprising the nucleotide sequence of the full-length protein coding sequence of clone yb7_{—}1 deposited with the ATCC under accession number 98600;

(d) a polynucleotide encoding the full-length protein encoded by the cDNA insert of clone yb7_{—}1 deposited with the ATCC under accession number 98600;

(e) a polynucleotide comprising the nucleotide sequence of a mature protein coding sequence of clone yb7_{—}1 deposited with the ATCC under accession number 98600;

(f) a polynucleotide encoding a mature protein encoded by the cDNA insert of clone yb7_{—}1 deposited with the ATCC under accession number 98600;

(g) a polynucleotide encoding a protein comprising the amino acid sequence of SEQ ID NO:40;

(h) a polynucleotide encoding a protein comprising a fragment of the amino acid sequence of SEQ ID NO:40 having biological activity, the fragment comprising eight consecutive amino acids of SEQ ID NO:40;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:39 from nucleotide 843 to nucleotide 1004; the nucleotide sequence of the full-length protein coding sequence of clone yb7_{—}1 deposited with the ATCC under accession number 98600; or the nucleotide sequence of a mature protein coding sequence of clone yb7_{—}1 deposited with the ATCC under accession number 98600. In other preferred embodiments, the polynucleotide encodes the full-length or a mature protein encoded by the cDNA insert of clone yb7_{—}1 deposited with the ATCC under accession number 98600.

In further preferred embodiments, the present invention provides a polynucleotide encoding a protein comprising a fragment of the amino acid sequence of SEQ ID NO:40 having biological activity, the fragment preferably comprising eight (more preferably twenty, most preferably thirty) consecutive amino acids of SEQ ID NO:40, or a polynucleotide encoding a protein comprising a fragment of the amino acid sequence of SEQ ID NO:40 having biological activity, the fragment comprising the amino acid sequence from amino acid 22 to amino acid 31 of SEQ ID NO:40.

Other embodiments provide the gene corresponding to the cDNA sequence of SEQ ID NO:39.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:39, but excluding the poly(A) tail at the 3′ end of SEQ ID NO:39; and

(ab) the nucleotide sequence of the cDNA insert of clone yb7_{—}1 deposited with the ATCC under accession number 98600; and

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:39, but excluding the poly(A) tail at the 3′ end of SEQ ID NO:39; and

(bb) the nucleotide sequence of the cDNA insert of clone yb7_{—}1 deposited with the ATCC under accession number 98600; and

(ii) hybridizing said primer(s) to human DNA;

(iii) amplifying human DNA sequences; and

(iv) isolating the polynucleotide product of step (b)(iii).

Preferably the polynucleotide isolated according to the above process comprises a nucleotide sequence corresponding to the cDNA sequence of SEQ ID NO:39, and extending contiguously from a nucleotide sequence corresponding to the 5′ end of SEQ ID NO:39 to a nucleotide sequence corresponding to the 3′ end of SEQ ID NO:39, but excluding the poly(A) tail at the 3′ end of SEQ ID NO:39. Also preferably the polynucleotide isolated according to the above process comprises a nucleotide sequence corresponding to the cDNA sequence of SEQ ID NO:39 from nucleotide 843 to nucleotide 1004, and extending contiguously from a nucleotide sequence corresponding to the 5′ end of said sequence of SEQ ID NO:39 from nucleotide 843 to nucleotide 1004, to a nucleotide sequence corresponding to the 3′ end of said sequence of SEQ ID NO:39 from nucleotide 843 to nucleotide 1004.

In other embodiments, the present invention provides a composition comprising a protein, wherein said protein comprises an amino acid sequence selected from the group consisting of:

(a) the amino acid sequence of SEQ ID NO:40;

(b) fragments of the amnino acid sequence of SEQ ID NO:40 comprising eight consecutive amino acids of SEQ ID NO:40; and

(c) the amino acid sequence encoded by the cDNA insert of clone yb7_{—}1 deposited with the ATCC under accession number 98600; the protein being substantially free from other mammalian proteins. Preferably such protein comprises the amino acid sequence of SEQ ID NO:40. In further preferred embodiments, the present invention provides a protein comprising a fragment of the amnino acid sequence of SEQ ID NO:40 having biological activity, the fragment preferably comprising eight (more preferably twenty, most preferably thirty) consecutive amino acids of SEQ ID NO:40, or a protein comprising a fragment of the amino acid sequence of SEQ ID NO:40 having biological activity, the fragment comprising the arnino acid sequence from amino acid 22 to amino acid 31 of SEQ ID NO:40.

In one embodiment, the present invention provides a composition comprising an isolated polynucleotide selected from the group consisting of:

(a) a polynucleotide comprising the nucleotide sequence of SEQ ID NO:41;

(b) a polynucleotide comprising the nucleotide sequence of SEQ ID NO:41 from nucleotide 179 to nucleotide 4285;

(c) a polynucleotide comprising the nucleotide sequence of the full-length protein coding sequence of clone am728_{—}60 deposited with the ATCC under accession number 98621;

(d) a polynucleotide encoding the full-length protein encoded by the cDNA insert of clone am728_{—}60 deposited with the ATCC under accession number 98621;

(e) a polynucleotide comprising the nucleotide sequence of a mature protein coding sequence of clone am728_{—}60 deposited with the ATCC under accession number 98621;

(e) a polynucleotide encoding a mature protein encoded by the cDNA insert of clone am728_{—}60 depositedwiththeATCC underaccessionnumber98621;

(g) a polynucleotide encoding a protein comprising the amino acid sequence of SEQ ID NO:42;

(h) a polynucleotide encoding a protein comprising a fragment of the amino acid sequence of SEQ ID NO:42 having biological activity, the fragment comprising eight consecutive amino acids of SEQ ID NO:42;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:41 from nucleotide 179 to nucleotide 4285; the nucleotide sequence of the full-length protein coding sequence of clone am728_{—}60 deposited with the ATCC under accession number 98621; or the nucleotide sequence of a mature protein coding sequence of clone am728_{—}60 deposited with the ATCC under accession number 98621. In other preferred embodiments, the polynucleotide encodes the full-length or a mature protein encoded by the cDNA insert of clone am728_{—}60 deposited with the ATCC under accession number 98621. In further preferred embodiments, the present invention provides a polynucleotide encoding a protein comprising a fragment of the amino acid sequence of SEQ ID NO:42 having biological activity, the fragment preferably comprising eight (more preferably twenty, most preferably thirty) consecutive amino acids of SEQ ID NO:42, or a polynucleotide encoding a protein comprising a fragment of the amino acid sequence of SEQ ID NO:42 having biological activity, the fragment comprising the amino acid sequence from amino acid 679 to amino acid 688 of SEQ ID NO:42.

Other embodiments provide the gene corresponding to the cDNA sequence of SEQ ID NO:41.

Further embodiments of the invention provide isolated polynucleotides produced according to a process selected from the group consisting of:

(a) a process comprising the steps of:

(i) preparing one or more polynucleotide probes that hvbridize in 6×SSC at 65 degrees C to a nucleotide sequence selected from the group consisting of:

(aa) SEQ ID NO:41; and

(ab) the nucleotide sequence of the cDNA insert of clone am728_{—}60 deposited with the ATCC under accession number 98621;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:41; and

(bb) the nucleotide sequence of the cDNA insert of clone am728_{—}60 deposited with the ATCC under accession number 98621;

(ii) hybridizing said primer(s) to human genornic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:41, and extending contiguously from a nucleotide sequence corresponding to the 5′ end of SEQ ID NO:41 to a nucleotide sequence corresponding to the 3′ end of SEQ ID NO:41 . Also preferably the polynucleotide isolated according to the above process comprises a nucleotide sequence corresponding to the cDNA sequence of SEQ ID NO:41 from nucleotide 179 to nucleotide 4285, and extending contiguously from a nucleotide sequence corresponding to the 5′ end of said sequence of SEQ ID NO:41 from nucleotide 179 to nucleotide 4285, to a nucleotide sequence corresponding to the 3′ end of said sequence of SEQ ID NO:41 from nucleotide 179 to nucleotide 4285.

In other embodiments, the present invention provides a composition comprising a protein, wherein said protein comprises an amnino acid sequence selected from the group consisting of:

(a) the amino acid sequence of SEQ ID NO:42;

(b) fragments of the amino acid sequence of SEQ ID NO:42, each fragment comprising eight consecutive amino acids of SEQ ID NO:42; and

(c) the amino acid sequence encoded by the cDNA insert of clone am728_{—}60 deposited with the ATCC under accession number 98621;

the protein being substantially free from other mammalian proteins. Preferably such protein comprises the amino acid sequence of SEQ ID NO:42. In further preferred embodiments, the present invention provides a protein comprising a fragment of the amino acid sequence of SEQ ID NO:42 having biological activity, the fragment preferably comprising eight (more preferably twenty, most preferably thirty) consecutive amino acids of SEQ ID NO:42, or a protein comprising a fragment of the amino acid sequence of SEQ ID NO:42 having biological activity, the fragment comprising the amino acid sequence from amino acid 679 to arnino acid 688 of SEQ ID NO:42.

In one embodiment, the present invention provides a composition comprising an isolated polynucleotide selected from the group consisting of:

(a) a polynucleotide comprising the nucleotide sequence of SEQ ID NO:43;

(b) a polynucleotide comprising the nucleotide sequence of SEQ ID NO:43 from nucleotide 108 to nucleotide 254;

(c) a polynucleotide comprising the nucleotide sequence of SEQ ID NO:43 from nucleotide 225 to nucleotide 254;

(d) a polynucleotide comprising the nucleotide sequence of the full-length protein coding sequence of clone bf377_{—}1 deposited with the ATCC under accession number 98621;

(e) a polynucleotide encoding the full-length protein encoded by the cDNA insert of clone bf377_{—}1 deposited with the ATCC under accession number 98621;

(f) a polynucleotide comprising the nucleotide sequence of a mature protein coding sequence of clone bf377_{—}1 deposited with the ATCC under accession number 98621;

(g) a polynucleotide encoding a mature protein encoded by the cDNA insert of clone bf377_{—}1 deposited with the ATCC under accession number 98621;

(h) a polynucleotide encoding a protein comprising the amino acid sequence of SEQ ID NO:44;

(i) a polynucleotide encoding a protein comprising a fragment of the amino acid sequence of SEQ ID NO:44 having biological activity, the fragment comprising eight consecutive amino acids of SEQ ID NO:44;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:43 from nucleotide 108 to nucleotide 254; the nucleotide sequence of SEQ ID NO:43 from nucleotide 225 to nucleotide 254; the nucleotide sequence of the full-length protein coding sequence of clone bf377_{—}1 deposited with the ATCC under accession number 98621; or the nucleotide sequence of a mature protein coding sequence of clone bf377_{—}1 deposited with the ATCC under accession number 98621. In other preferred embodiments, the polynucleotide encodes the full-length or a mature protein encoded by the cDNA insert of clone bf377_{—}1 deposited with the ATCC under accession number 98621. In further preferred embodiments, the present invention provides a polynucleotide encoding a protein comprising a fragment of the amino acid sequence of SEQ ID NO:44 having biological activity, the fragment preferably comprising eight (more preferably twenty, most preferably thirty) consecutive amino acids of SEQ ID NO:44, or a polynucleotide encoding a protein comprising a fragment of the amino acid sequence of SEQ ID NO:44 having biological activity, the fragment comprising the amino acid sequence from amino acid 19 to amino acid 28 of SEQ ID NO:44.

Other embodiments provide the gene corresponding to the cDNA sequence of SEQ ID NO:43.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:43, but excluding the poly(A) tail at the 3′ end of SEQ ID NO:43; and

(ab) the nucleotide sequence of the cDNA insert of clone bf377_{—}1 deposited with the ATCC under accession number 98621;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:43, but excluding the poly(A) tail at the 3′ end of SEQ ID NO:43; and

(bb) the nucleotide sequence of the cDNA insert of clone bf377_{—}1 deposited with the ATCC under accession number 98621;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:43, and extending contiguously from a nucleotide sequence corresponding to the 5′ end of SEQ ID NO:43 to a nucleotide sequence corresponding to the 3′ end of SEQ ID NO:43, but excluding the poly(A) tail at the 3′ end of SEQ ID NO:43. Also preferably the polynucleotide isolated according to the above process comprises a nucleotide sequence corresponding to the cDNA sequence of SEQ ID NO:43 from nucleotide 108 to nucleotide 254, and extending contiguously from a nucleotide sequence corresponding to the 5′ end of said sequence of SEQ ID NO:43 from nucleotide 108 to nucleotide 254, to a nucleotide sequence corresponding to the 3′ end of said sequence of SEQ ID NO:43 from nucleotide 108 to nucleotide 254. Also preferably the polynucleotide isolated according to the above process comprises a nucleotide sequence corresponding to the cDNA sequence of SEQ ID NO:43 from nucleotide 225 to nucleotide 254, and extending contiguously from a nucleotide sequence corresponding to the 5′ end of said sequence of SEQ ID NO:43 from nucleotide 225 to nucleotide 254, to a nucleotide sequence corresponding to the 3′ end of said sequence of SEQ ID NO:43 from nucleotide 225 to nucleotide 254.

In other embodiments, the present invention provides a composition comprising a protein, wherein said protein comprises an amino acid sequence selected from the group consisting of:

(a) the amino acid sequence of SEQ ID NO:44;

(b) fragments of the amino acid sequence of SEQ ID NO:44, each fragment comprising eight consecutive amino acids of SEQ ID NO:44; and

(c) the amino acid sequence encoded by the cDNA insert of clone bf377_{—}1 deposited with the ATCC under accession number 98621;

the protein being substantially free from other mammalian proteins. Preferably such protein comprises the amino acid sequence of SEQ ID NO:44. In further preferred embodiments, the present invention provides a protein comprising a fragment of the amino acid sequence of SEQ ID NO:44 having biological activity, the fragment preferably comprising eight (more preferably twenty, most preferably thirty) consecutive amino acids of SEQ ID NO:44, or a protein comprising a fragment of the amino acid sequence of SEQ ID NO:44 having biological activity, the fragment comprising the amino acid sequence from amino acid 19 to amino acid 28 of SEQ ID NO:44.

In one embodiment, the present invention provides a composition comprising an isolated polynucleotide selected from the group consisting of:

(a) a polynucleotide comprising the nucleotide sequence of SEQ ID NO:45;

(b) a polynucleotide comprising the nucleotide sequence of SEQ ID NO:45 from nucleotide 426 to nucleotide 569;

(c) a polynucleotide comprising the nucleotide sequence of SEQ ID NO:45 from nucleotide 546 to nucleotide 569;

(d) a polynucleotide comprising the nucleotide sequence of the full-length protein coding sequence of clone cw354_{—}1 deposited with the ATCC under accession number 98621;

(e) a polynucleotide encoding the full-length protein encoded by the cDNA insert of clone cw354_{—}1 deposited with the ATCC under accession number 98621;

(f) a polynucleotide comprising the nucleotide sequence of a mature protein coding sequence of clone cw354_{—}1 deposited with the ATCC under accession number 98621;

(g) a polynucleotide encoding a mature protein encoded by the cDNA insert of clone cw354_{—}1 deposited with the ATCC under accession number 98621;

(h) a polynucleotide encoding a protein comprising the arnino acid sequence of SEQ ID NO:46;

(i) a polynucleotide encoding a protein comprising a fragment of the amino acid sequence of SEQ ID NO:46 having biological activity, the fragment comprising eight consecutive amino acids of SEQ ID NO:46;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:45 from nucleotide 426 to nucleotide 569; the nucleotide sequence of SEQ ID NO:45 from nucleotide 546 to nucleotide 569; the nucleotide sequence of the full-length protein coding sequence of clone cw354_{—}1 deposited with the ATCC under accession number 98621; or the nucleotide sequence of a mature protein coding sequence of clone cw354_{—}1 deposited with the ATCC under accession number 98621. In other preferred embodiments, the polynucleotide encodes the full-length or a mature protein encoded by the cDNA insert of clone cw354_{—}1 deposited with the ATCC under accession number 98621. In further preferred embodiments, the present invention provides a polynucleotide encoding a protein comprising a fragment of the amino acid sequence of SEQ ID NO:46 having biological activity, the fragment preferably comprising eight (more preferably twenty, most preferably thirty) consecutive amino acids of SEQ ID NO:46, or a polynucleotide encoding a protein comprising a fragment of the amino acid sequence of SEQ ID NO:46 having biological activity, the fragment comprising the amino acid sequence from amino acid 19 to amino acid 28 of SEQ ID NO:46.

Other embodiments provide the gene corresponding to the cDNA sequence of SEQ ID NO:45.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:45, but excluding the poly(A) tail at the 3′ end of SEQ ID NO:45; and

(ab) the nucleotide sequence of the cDNA insert of clone cw354_{—}1 deposited with the ATCC under accession number 98621;

(ii) hybridizing said probe(s) to human genorric DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:45, but excluding the poly(A) tail at the 3′ end of SEQ ID NO:45; and

(bb) the nucleotide sequence of the cDNA insert of clone cw354_{—}1 deposited with the ATCC under accession number 98621;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a 9nucleotide sequence corresponding to the cDNA sequence of SEQ ID NO:45, and extending contiguously from a nucleotide sequence corresponding to the 5′ end of SEQ ID NO:45 to a nucleotide sequence corresponding to the 3′ end of SEQ ID NO:45, but excluding the poly(A) tail at the 3′ end of SEQ ID NO:45. Also preferably the polynucleotide isolated according to the above process comprises a nucleotide sequence corresponding to the cDNA sequence of SEQ ID NO:45 from nucleotide 426 to nucleotide 569, and extending contiguously from a nucleotide sequence corresponding to the 5′ end of said sequence of SEQ ID NO:45 from nucleotide 426 to nucleotide 569, to a nucleotide sequence corresponding to the 3′ end of said sequence of SEQ ID NO:45 from nucleotide 426 to nucleotide 569. Also preferably the polynucleotide isolated according to the above process comprises a nucleotide sequence corresponding to the cDNA sequence of SEQ ID NO:45 from nucleotide 546 to nucleotide 569, and extending contiguously from a nucleotide sequence corresponding to the 5′ end of said sequence of SEQ ID NO:45 from nucleotide 546 to nucleotide 569, to a nucleotide sequence corresponding to the 3′ end of said sequence of SEQ ID NO:45 from nucleotide 546 to nucleotide 569.

In other embodiments, the present invention provides a composition comprising a protein, wherein said protein comprises an amino acid sequence selected from the group consisting of:

(a) the amino acid sequence of SEQ ID NO:46;

(b) fragments of the amino acid sequence of SEQ ID NO:46, each fragment comprising eight consecutive amino acids of SEQ ID NO:46; and

(c) the amino acid sequence encoded by the cDNA insert of clone cw354_{—}1 deposited with the ATCC under accession number 98621;

the protein being substantially free from other mammalian proteins. Preferably such protein comprises the amino acid sequence of SEQ ID NO:46. In further preferred embodiments, the present invention provides a protein comprising a fragment of the amino acid sequence of SEQ ID NO:46 having biological activity, the fragment preferably comprising eight (more preferably twenty, most preferably thirty) consecutive amino acids of SEQ ID NO:46, or a protein comprising a fragment of the amino acid sequence of SEQ ID NO:46 having biological activity, the fragment comprising the amino acid sequence from amino acid 19 to amino acid 28 of SEQ ID NO:46.

In one embodiment, the present invention provides a composition comprising an isolated polynucleotide selected from the group consisting of:

(a) a polynucleotide comprising the nucleotide sequence of SEQ ID NO:47;

(b) a polynucleotide comprising the nucleotide sequence of SEQ ID NO:47 from nucleotide 151 to nucleotide 891;

(c) a polynucleotide comprising the nucleotide sequence of SEQ ID NO:47 from nucleotide 595 to nucleotide 891;

(d) a polynucleotide comprising the nucleotide sequence of the full-length protein coding sequence of clone nm134_{—}4 deposited with the ATCC under accession number 98621;

(e) a polynucleotide encoding the full-length protein encoded by the cDNA insert of clone nm134_{—}4 deposited with the ATCC under accession number 98621;

(f) a polynucleotide comprising the nucleotide sequence of a mature protein coding sequence of clone nm134_{—}4 deposited with the ATCC under accession number 98621;

(g) a polynucleotide encoding a mature protein encoded by the cDNA insert of clone nm134_{—}4 deposited with the ATCC under accession number 98621;

(h) a polynucleotide encoding a protein comprising the amino acid sequence of SEQ ID NO:48;

(i) a polynucleotide encoding a protein comprising a fragment of the amino acid sequence of SEQ ID NO:48 having biological activity, the fragment comprising eight consecutive amino acids of SEQ ID NO:48;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:47 from nucleotide 151 to nucleotide 891; the nucleotide sequence of SEQ ID NO:47 from nucleotide 595 to nucleotide 891; the nucleotide sequence of the full-length protein coding sequence of clone nm134_{—}4 deposited with the ATCC under accession number 98621; or the nucleotide sequence of a mature protein coding sequence of clone nm134_{—}4 deposited with the ATCC under accession number 98621. In other preferred embodiments, the polynucleotide encodes the full-length or a mature protein encoded by the cDNA insert of clone nm134_{—}4 deposited with the ATCC under accession number 98621. In yet other preferred embodiments, the present invention provides a polynucleotide encoding a protein comprising the amino acid sequence of SEQ ID NO:48 from amino acid 104 to amino acid 163. In further preferred embodiments, the present invention provides a polynucleotide encoding a protein comprising a fragment of the amino acid sequence of SEQ ID NO:48 having biological activity, the fragment preferably comprising eight (more preferably twenty, most preferably thirty) consecutive amino acids of SEQ ID NO:48, or a polynucleotide encoding a protein comprising a fragment of the amino acid sequence of SEQ ID NO:48 having biological activity, the fragment comprising the amino acid sequence from amino acid 118 to amino acid 127 of SEQ ID NO:48.

Other embodiments provide the gene corresponding to the cDNA sequence of SEQ ID NO:47.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:47, but excluding the poly(A) tail at the 3′ end of SEQ ID NO:47; and

(ab) the nucleotide sequence of the cDNA insert of clone nm134_{—}4 deposited with the ATCC under accession number 98621;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:47, but excluding the poly(A) tail at the 3′ end of SEQ ID NO:47; and

(bb) the nucleotide sequence of the cDNA insert of clone nm134_{—}4 deposited with the ATCC under accession number 98621;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:47, and extending contiguously from a nucleotide sequence corresponding to the 5′ end of SEQ ID NO:47 to a nucleotide sequence corresponding to the 3′ end of SEQ ID NO:47, but excluding the poly(A) tail at the 3′ end of SEQ ID NO:47. Also preferably the polynucleotide isolated according to the above process comprises a nucleotide sequence corresponding to the cDNA sequence of SEQ ID NO:47 from nucleotide 151 to nucleotide 891, and extending contiguously from a nucleotide sequence corresponding to the 5′ end of said sequence of SEQ ID NO:47 from nucleotide 151 to nucleotide 891, to a nucleotide sequence corresponding to the 3′ end of said sequence of SEQ ID NO:47 from nucleotide 151 to nucleotide 891. Also preferably the polynucleotide isolated according to the above process comprises a nucleotide sequence corresponding to the cDNA sequence of SEQ ID NO:47 from nucleotide 595 to nucleotide 891, and extending contiguously from a nucleotide sequence corresponding to the 5′ end of said sequence of SEQ ID NO:47 from nucleotide 595 to nucleotide 891, to a nucleotide sequence corresponding to the 3′ end of said sequence of SEQ ID NO:47 from nucleotide 595 to nucleotide 891.

In other embodiments, the present invention provides a composition comprising a protein, wherein said protein comprises an amino acid sequence selected from the group consisting of:

(a) the amino acid sequence of SEQ ID NO:48;

(b) the amino acid sequence of SEQ ID NO:48 from amino acid 104 to amino acid 163;

(c) fragments of the amino acid sequence of SEQ ID NO:48, each fragment comprising eight consecutive amino acids of SEQ ID NO:48; and

(d) the amnino acid sequence encoded by the cDNA insert of clone nm134_{—}4 deposited with the ATCC under accession number 98621;

the protein being substantially free from other mammalian proteins. Preferably such protein comprises the amino acid sequence of SEQ ID NO:48 or the amino acid sequence of SEQ ID NO:48 from amino acid 104 to amino acid 163. In further preferred embodiments, the present invention provides a protein comprising a fragment of the amino acid sequence of SEQ ID NO:48 having biological activity, the fragment preferably comprising eight (more preferably twenty, most preferably thirty) consecutive amino acids of SEQ ID NO:48, or a protein comprising a fragment of the amino acid sequence of SEQ ID NO:48 having biological activity, the fragment comprising the amino acid sequence from amino acid 118 to amino acid 127 of SEQ ID NO:48.

In one embodiment, the present invention provides a composition comprising an isolated polynucleotide selected from the group consisting of:

(a) a polynucleotide comprising the nucleotide sequence of SEQ ID NO:49;

(b) a polynucleotide comprising the nucleotide sequence of SEQ ID NO:49 from nucleotide 1909 to nucleotide 2127;

(c) a polynucleotide comprising the nucleotide sequence of SEQ ID NO:49 from nucleotide 2074 to nucleotide 2127;

(d) a polynucleotide comprising the nucleotide sequence of the full-length protein coding sequence of clone yb11_{—}1 deposited with the ATCC under accession number 98621;

(e) a polynucleotide encoding the full-length protein encoded by the cDNA insert of clone yb11_{—}1 deposited with the ATCC under accession number 98621;

(f) a polynucleotide comprising the nucleotide sequence of a mature protein coding sequence of clone yb11_{—}1 deposited with the ATCC under accession number 98621;

(g) a polynucleotide encoding a mature protein encoded by the cDNA insert of clone yb11_{—}1 deposited with the ATCC under accession number 98621;

(h) a polynucleotide encoding a protein comprising the amino acid sequence of SEQ ID NO:50;

(i) a polynucleotide encoding a protein comprising a fragment of the amino acid sequence of SEQ ID NO:50 having biological activity, the fragment comprising eight consecutive amino acids of SEQ ID NO:50;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:49 from nucleotide 1909 to nucleotide 2127; the nucleotide sequence of SEQ ID NO:49 from nucleotide 2074 to nucleotide 2127; the nucleotide sequence of the full-length protein coding sequence of clone yb11_{—}1 deposited with the ATCC under accession number 98621; or the nucleotide sequence of a mature protein coding sequence of clone yb11_{—}1 deposited with the ATCC under accession number 98621. In other preferred embodiments, the polynucleotide encodes the full-length or a mature protein encoded by the cDNA insert of clone yb11_{—}1 deposited with the ATCC under accession number 98621. In further preferred embodiments, the present invention provides a polynucleotide encoding a protein comprising a fragment of the amino acid sequence of SEQ ID NO:50 having biological activity, the fragment preferably comprising eight (more preferably twenty, most preferably thirty) consecutive amino acids of SEQ ID NO:50, or a polynucleotide encoding a protein comprising a fragment of the amino acid sequence of SEQ ID NO:50 having biological activity, the fragment comprising the amino acid sequence from amino acid 31 to amino acid 40 of SEQ ID NO:50.

Other embodiments provide the gene corresponding to the cDNA sequence of SEQ ID NO:49.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:49, but excluding the poly(A) tail at the 3′ end of SEQ ID NO:49; and

(ab) the nucleotide sequence of the cDNA insert of clone yb11_{—}1 deposited with the ATCC under accession number 98621;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:49, but excluding the poly(A) tail at the 3′ end of SEQ ID NO:49; and

(bb) the nucleotide sequence of the cDNA insert of clone ybll_{—}1 deposited with the ATCC under accession number 98621;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:49, and extending contiguously from a nucleotide sequence corresponding to the 5′ end of SEQ ID NO:49 to a nucleotide sequence corresponding to the 3′ end of SEQ ID NO:49, but excluding the poly(A) tail at the 3′ end of SEQ ID NO:49. Also preferably the polynucleotide isolated according to the above process comprises a nucleotide sequence corresponding to the cDNA sequence of SEQ ID NO:49 from nucleotide 1909 to nucleotide 2127, and extending contiguously from a nucleotide sequence corresponding to the 5′ end of said sequence of SEQ ID NO:49 from nucleotide 1909 to nucleotide 2127, to a nucleotide sequence corresponding to the 3′ end of said sequence of SEQ ID NO:49 from nucleotide 1909 to nucleotide 2127. Also preferably the polynucleotide isolated according to the above process comprises a nucleotide sequence corresponding to the cDNA sequence of SEQ ID NO:49 from nucleotide 2074 to nucleotide 2127, and extending contiguously from a nucleotide sequence corresponding to the 5′ end of said sequence of SEQ ID NO:49 from nucleotide 2074 to nucleotide 2127, to a nucleotide sequence corresponding to the 3′ end of said sequence of SEQ ID NO:49 from nucleotide 2074 to nucleotide 2127.

In other embodiments, the present invention provides a composition comprising a protein, wherein said protein comprises an amino acid sequence selected from the group consisting of:

(a) the amino acid sequence of SEQ ID NO:50;

(b) fragments of the amino acid sequence of SEQ ID NO:50, each fragment comprising eight consecutive amino acids of SEQ ID NO:50; and

(c) the amino acid sequence encoded by the cDNA insert of clone yb11_{—}1 deposited with the ATCC under accession number 98621;

the protein being substantially free from other mammalian proteins. Preferably such protein comprises the amino acid sequence of SEQ ID NO:50. In further preferred embodiments, the present invention provides a protein comprising a fragment of the amino acid sequence of SEQ ID NO:50 having biological activity, the fragment preferably comprising eight (more preferably twenty, most preferably thirty) consecutive amino acids of SEQ ID NO:50, or a protein comprising a fragment of the amino acid sequence of SEQ ID NO:50 having biological activity, the fragment comprising the amino acid sequence from amino acid 31 to amino acid 40 of SEQ ID NO:50.

In one embodiment, the present invention provides a composition comprising an isolated polynucleotide selected from the group consisting of:

(a) a polynucleotide comprising the nucleotide sequence of SEQ ID NO:51;

(b) a polynucleotide comprising the nucleotide sequence of SEQ ID NO:51 from nucleotide 1077 to nucleotide 1733;

(c) a polynucleotide comprising the nucleotide sequence of SEQ ID NO:51 from nucleotide 1158 to nucleotide 1733;

(d) a polynucleotide comprising the nucleotide sequence of the full-length protein coding sequence of clone yc2_{—}1 deposited with the ATCC under accession number 98621;

(e) a polynucleotide encoding the full-length protein encoded by the cDNA insert of clone yc2_{—}1 deposited with the ATCC under accession number 98621;

(f) a polynucleotide comprising the nucleotide sequence of a mature protein coding sequence of clone yc2_{—}1 deposited with the ATCC under accession number 98621;

(g) a polynucleotide encoding a mature protein encoded by the cDNA insert of clone yc2_{—}1 deposited with the ATCC under accession number 98621;

(h) a polynucleotide encoding a protein comprising the amino acid sequence of SEQ ID NO:52;

(i) a polynucleotide encoding a protein comprising a fragment of the amino acid sequence of SEQ ID NO:52 having biological activity, the fragment comprising eight consecutive amino acids of SEQ ID NO:52;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:51 from nucleotide 1077 to nucleotide 1733; the nucleotide sequence of SEQ ID NO:51 from nucleotide 1158 to nucleotide 1733; the nucleotide sequence of the full-length protein coding sequence of clone yc2_{—}1 deposited with the ATCC under accession number 98621; or the nucleotide sequence of a mature protein coding sequence of clone yc2_{—}1 deposited with the ATCC under accession number 98621. In other preferred embodiments, the polynucleotide encodes the full-length or a mature protein encoded by the cDNA insert of clone yc2_{—}1 deposited with the ATCC under accession number 98621.

In further preferred embodiments, the present invention provides a polynucleotide encoding a protein comprising a fragment of the amino acid sequence of SEQ ID NO:52 having biological activity, the fragment preferably comprising eight (more preferably twenty, most preferably thirty) consecutive amino acids of SEQ ID NO:52, or a polynucleotide encoding a protein comprising a fragment of the amino acid sequence of SEQ ID NO:52 having biological activity, the fragment comprising the amino acid sequence from amino acid 104 to amino acid 113 of SEQ ID NO:52.

Other embodiments provide the gene corresponding to the cDNA sequence of SEQ ID NO:51.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:51, but excluding the poly(A) tail at the 3′ end of SEQ ID NO:51; and

(ab) the nucleotide sequence of the cDNA insert of clone yc2_{—}1 deposited with the ATCC under accession number 98621;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:51, but excluding the poly(A) tail at the 3′ end of SEQ ID NO:51; and

(bb) the nucleotide sequence of the cDNA insert of clone yc2_{—}1 deposited with the ATCC under accession number 98621;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:51, and extending contiguously from a nucleotide sequence corresponding to the 5′ end of SEQ ID NO:51 to a nucleotide sequence corresponding to the 3′ end of SEQ ID NO:51 , but excluding the poly(A) tail at the 3′ end of SEQ ID NO:51. Also preferably the polynucleotide isolated according to the above process comprises a nucleotide sequence corresponding to the cDNA sequence of SEQ ID NO:51 from nucleotide 1077 to nucleotide 1733, and extending contiguously from a nucleotide sequence corresponding to the 5′ end of said sequence of SEQ ID NO:51 from nucleotide 1077 to nucleotide 1733, to a nucleotide sequence corresponding to the 3′ end of said sequence of SEQ ID NO:51 from nucleotide 1077 to nucleotide 1733. Also preferably the polynucleotide isolated according to the above process comprises a nucleotide sequence corresponding to the cDNA sequence of SEQ ID NO:51 from nucleotide 1158 to nucleotide 1733, and extending contiguously from a nucleotide sequence corresponding to the 5′ end of said sequence of SEQ ID NO:51 from nucleotide 1158 to nucleotide 1733, to a nucleotide sequence corresponding to the 3′ end of said sequence of SEQ ID NO:51 from nucleotide 1158 to nucleotide 1733.

In other embodiments, the present invention provides a composition comprising a protein, wherein said protein comprises an amino acid sequence selected from the group consisting of:

(a) the amino acid sequence of SEQ ID NO:52;

(b) fragments of the amino acid sequence of SEQ ID NO:52, each fragment comprising eight consecutive amino acids of SEQ ID NO:52; and

(c) the amino acid sequence encoded by the cDNA insert of clone yc2_{—}1 deposited with the ATCC under accession number 98621;

the protein being substantially free from other mammalian proteins. Preferably such protein comprises the amino acid sequence of SEQ ID NO:52. In further preferred embodiments, the present invention provides a protein comprising a fragment of the amino acid sequence of SEQ ID NO:52 having biological activity, the fragment preferably comprising eight (more preferably twenty, most preferably thirty) consecutive amino acids of SEQ ID NO:52, or a protein comprising a fragment of the amino acid sequence of SEQ ID NO:52 having biological activity, the fragment comprising the amino acid sequence from amino acid 104 to amino acid 113 of SEQ ID NO:52.

In one embodiment, the present invention provides a composition comprising an isolated polynucleotide selected from the group consisting of:

(a) a polynucleotide comprising the nucleotide sequence of SEQ ID NO:53;

(b) a polynucleotide comprising the nucleotide sequence of SEQ ID NO:53 from nucleotide 257 to nucleotide 622;

(c) a polynucleotide comprising the nucleotide sequence of the full-length protein coding sequence of clone ff168_{—}12 deposited with the ATCC under accession number 98623;

(d) a polynucleotide encoding the full-length protein encoded by the cDNA insert of clone ff168_{—}12 deposited with the ATCC under accession number 98623;

(e) a polynucleotide comprising the nucleotide sequence of a mature protein coding sequence of clone ff168_{—}12 deposited with the ATCC under accession number 98623;

(f) a polynucleotide encoding a mature protein encoded by the cDNA insert of clone ff168_{—}12 deposited with the ATCC under accession number 98623;

(g) a polynucleotide encoding a protein comprising the amnino acid sequence of SEQ ID NO:54;

(h) a polynucleotide encoding a protein comprising a fragment of the amino acid sequence of SEQ ID NO:54 having biological activity, the fragment comprising eight consecutive amino acids of SEQ ID NO:54;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:53 from nucleotide 257 to nucleotide 622; the nucleotide sequence of the full-ength protein coding sequence of clone ff168_{—}12 deposited with the ATCC under accession number 98623; or the nucleotide sequence of a mature protein coding sequence of clone ff168_{—}12 deposited with the ATCC under accession number 98623. In other preferred embodiments, the polynucleotide encodes the full-length or a mature protein encoded by the cDNA insert of clone ff168_{—}12 deposited with the ATCC under accession number 98623. In further preferred embodiments, the present invention provides a polynucleotide encoding a protein comprising a fragment of the amino acid sequence of SEQ ID NO:54 having biological activity, the fragment preferably comprising eight (more preferably twenty, most preferably thirty) consecutive amino acids of SEQ ID NO:54, or a polynucleotide encoding a protein comprising a fragment of the amino acid sequence of SEQ ID NO:54 having biological activity, the fragment comprising the amino acid sequence from amino acid 56 to amino acid 65 of SEQ ID NO:54.

Other embodiments provide the gene corresponding to the cDNA sequence of SEQ ID NO:53.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:53, but excluding the poly(A) tail at the 3′ end of SEQ ID NO:53; and

(ab) the nucleotide sequence of the cDNA insert of clone ff168_{—}12 deposited with the ATCC under accession number 98623;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:53, but excluding the poly(A) tail at the 3′ end of SEQ ID NO:53; and

(bb) the nucleotide sequence of the cDNA insert of clone ff168_{—}12 deposited with the ATCC under accession number 98623;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:53, and extending contiguously from a nucleotide sequence corresponding to the 5′ end of SEQ ID NO:53 to a nucleotide sequence corresponding to the 3′ end of SEQ ID NO:53, but excluding the poly(A) tail at the 3′ end of SEQ ID NO:53. Also preferably the polynucleotide isolated according to the above process comprises a nucleotide sequence corresponding to the cDNA sequence of SEQ ID NO:53 from nucleotide 257 to nucleotide 622, and extending contiguously from a nucleotide sequence corresponding to the 5′ end of said sequence of SEQ ID NO:53 from nucleotide 257 to nucleotide 622, to a nucleotide sequence corresponding to the 3′ end of said sequence of SEQ ID NO:53 from nucleotide 257 to nucleotide 622.

In other embodiments, the present invention provides a composition comprising a protein, wherein said protein comprises an amino acid sequence selected from the group consisting of:

(a) the amino acid sequence of SEQ ID NO:54;

(b) fragments of the amino acid sequence of SEQ ID NO:54, each fragment comprising eight consecutive amino acids of SEQ ID NO:54; and

(c) the amino acid sequence encoded by the cDNA insert of clone ff168_{—}12 deposited with the ATCC under accession number 98623;

the protein being substantially free from other mammalian proteins. Preferably such protein comprises the amino acid sequence of SEQ ID NO:54. In further preferred embodiments, the present invention provides a protein comprising a fragment of the amino acid sequence of SEQ ID NO:54 having biological activity, the fragment preferably comprising eight (more preferably twenty, most preferably thirty) consecutive amino acids of SEQ ID NO:54, or a protein comprising a fragment of the amino acid sequence of SEQ ID NO:54 having biological activity, the fragment comprising the amino acid sequence from amino acid 56 to amino acid 65 of SEQ ID NO:54.

In one embodiment, the present invention provides a composition comprising an isolated polynucleotide selected from the group consisting of:

(a) a polynucleotide comprising the nucleotide sequence of SEQ ID NO:55;

(b) a polynucleotide comprising the nucleotide sequence of SEQ ID NO:55 from nucleotide 1323 to nucleotide 1829;

(c) a polynucleotide comprising the nucleotide sequence of SEQ ID NO:55 from nucleotide 1539 to nucleotide 1829;

(d) a polynucleotide comprising the nucleotide sequence of the full-length protein coding sequence of clone ls9_{—}1 deposited with the ATCC under accession number 98623;

(e) a polynucleotide encoding the full-length protein encoded by the cDNA insert of clone ls9_{—}1 deposited with the ATCC under accession number 98623;

(f) a polynucleotide comprising the nucleotide sequence of a mature protein coding sequence of clone ls9_{—}1 deposited with the ATCC under accession number 98623;

(g) a polynucleotide encoding a mature protein encoded by the cDNA insert of clone ls9_{—}1 deposited with the ATCC under accession number 98623;

(h) a polynucleotide encoding a protein comprising the amino acid sequence of SEQ ID NO:56;

(i) a polynucleotide encoding a protein comprising a fragment of the amino acid sequence of SEQ ID NO:56 having biological activity, the fragment comprising eight consecutive amino acids of SEQ ID NO:56;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:55 from nucleotide 1323 to nucleotide 1829; the nucleotide sequence of SEQ ID NO:55 from nucleotide 1539 to nucleotide 1829; the nucleotide sequence of the full-length protein coding sequence of clone ls9_{—}1 deposited with the ATCC under accession number 98623; or the nucleotide sequence of a mature protein coding sequence of clone ls9_{—}1 deposited with the ATCC under accession number 98623. In other preferred embodiments, the polynucleotide encodes the full-length or a mature protein encoded by the cDNA insert of clone ls9_{—}1 deposited with the ATCC under accession number 98623.

In further preferred embodiments, the present invention provides a polynucleotide encoding a protein comprising a fragment of the amino acid sequence of SEQ ID NO:56 having biological activity, the fragment preferably comprising eight (more preferably twenty, most preferably thirty) consecutive amino acids of SEQ ID NO:56, or a polynucleotide encoding a protein comprising a fragment of the amino acid sequence of SEQ ID NO:56 having biological activity, the fragment comprising the amino acid sequence from amino acid 79 to amino acid 88 of SEQ ID NO:56.

Other embodiments provide the gene corresponding to the cDNA sequence of SEQ ID NO:55.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:55, but excluding the poly(A) tail at the 3′ end of SEQ ID NO:55; and

(ab) the nucleotide sequence of the cDNA insert of clone ls9_{—}1 deposited with the ATCC under accession number 98623;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:55, but excluding the poly(A) tail at the 3′ end of SEQ ID NO:55; and

(bb) the nucleotide sequence of the cDNA insert of clone ls9_{—}1 deposited with the ATCC under accession number 98623;

(ii) hybridizing said primer(s) to human genomnic DNA in conditions at least as stringent as 4)(SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:55, and extending contiguously from a nucleotide sequence corresponding to the 5′ end of SEQ ID NO:55 to a nucleotide sequence corresponding to the 3′ end of SEQ ID NO:55, but excluding the poly(A) tail at the 3′ end of SEQ ID NO:55. Also preferably the polynucleotide isolated according to the above process comprises a nucleotide sequence corresponding to the cDNA sequence of SEQ ID NO:55 from nucleotide 1323 to nucleotide 1829, and extending contiguously from a nucleotide sequence corresponding to the 5′ end of said sequence of SEQ ID NO:55 from nucleotide 1323 to nucleotide 1829, to a nucleotide sequence corresponding to the 3′ end of said sequence of SEQ ID NO:55 from nucleotide 1323 to nucleotide 1829. Also preferably the polynucleotide isolated according to the above process comprises a nucleotide sequence corresponding to the cDNA sequence of SEQ ID NO:55 from nucleotide 1539 to nucleotide 1829, and extending contiguously from a nucleotide sequence corresponding to the 5′ end of said sequence of SEQ ID NO:55 from nucleotide 1539 to nucleotide 1829, to a nucleotide sequence corresponding to the 3′ end of said sequence of SEQ ID NO:55 from nucleotide 1539 to nucleotide 1829.

In other embodiments, the present invention provides a composition comprising a protein, wherein said protein comprises an amino acid sequence selected from the group consisting of:

(a) the amino acid sequence of SEQ ID NO:56;

(b) fragments of the amino acid sequence of SEQ ID NO:56, each fragment comprising eight consecutive amino acids of SEQ ID NO:56; and

(c) the amino acid sequence encoded by the cDNA insert of clone ls9_{—}1 deposited with the ATCC under accession number 98623;

the protein being substantially free from other mammalian proteins. Preferably such protein comprises the amino acid sequence of SEQ ID NO:56. In further preferred embodiments, the present invention provides a protein comprising a fragment of the amino acid sequence of SEQ ID NO:56 having biological activity, the fragment preferably comprising eight (more preferably twenty, most preferably thirty) consecutive amino acids of SEQ ID NO:56, or a protein comprising a fragment of the amino acid sequence of SEQ ID NO:56 having biological activity, the fragment comprising the amino acid sequence from amino acid 79 to amino acid 88 of SEQ ID NO:56.

In one embodiment, the present invention provides a composition comprising an isolated polynucleotide selected from the group consisting of:

(a) a polynucleotide comprising the nucleotide sequence of SEQ ID NO:57;

(b) a polynucleotide comprising the nucleotide sequence of SEQ ID NO:57 from nucleotide 507 to nucleotide 722;

(c) a polynucleotide comprising the nucleotide sequence of SEQ ID NO:57 from nucleotide 615 to nucleotide 722;

(d) a polynucleotide comprising the nucleotide sequence of the full-length protein coding sequence of clone na1010_{—}1 deposited with the ATCC under accession number 98623;

(e) a polynucleotide encoding the full-length protein encoded by the cDNA insert of clone na1010_{—}1 deposited with the ATCC under accession number 98623;

(f) a polynucleotide comprising the nucleotide sequence of a mature protein coding sequence of clone na1010_{—}1 deposited with the ATCC under accession number 98623;

(g) a poly-nucleotide encoding a mature protein encoded by the cDNA insert of clone na1010_{—}1 deposited with the ATCC under accession number 98623;

(h) a polynucleotide encoding a protein comprising the amino acid sequence of SEQ ID NO:58;

(i) a polynucleotide encoding a protein comprising a fragment of the amino acid sequence of SEQ ID NO:58 having biological activity, the fragment comprising eight consecutive arnino acids of SEQ ID NO:58;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:57 from nucleotide 507 to nucleotide 722; the nucleotide sequence of SEQ ID NO:57 from nucleotide 615 to nucleotide 722; the nucleotide sequence of the full-length protein coding sequence of clone na1010_{—}1 deposited with the ATCC under accession number 98623; or the nucleotide sequence of a mature protein coding sequence of clone na1010_{—}1 deposited with the ATCC under accession number 98623. In other preferred embodiments, the polynucleotide encodes the full-length or a mature protein encoded by the cDNA insert of clone na1010_{—}1 deposited with the ATCC under accession number 98623. In further preferred embodiments, the present invention provides a polynucleotide encoding a protein comprising a fragment of the amino acid sequence of SEQ ID NO:58 having biological activity, the fragment preferably comprising eight (more preferably twenty, most preferably thirty) consecutive amino acids of SEQ ID NO:58, or a polynucleotide encoding a protein comprising a fragment of the amino acid sequence of SEQ ID NO:58 having biological activity, the fragment comprising the amino acid sequence from amino acid 31 to amino acid 40 of SEQ ID NO:58.

Other embodiments provide the gene corresponding to the cDNA sequence of SEQ ID NO:57.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:57, but excluding the poly(A) tail at the 3′ end of SEQ ID NO:57; and

(ab) the nucleotide sequence of the cDNA insert of clone na1010_{—}1 deposited with the ATCC under accessionnumber 98623;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:57, but excluding the poly(A) tail at the 3′ end of SEQ ID NO:57; and

(bb) the nucleotide sequence of the cDNA insert of clone na1010_{—}1 deposited with the ATCC under accession number 98623;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:57, and extending contiguously from a nucleotide sequence corresponding to the 5′ end of SEQ ID NO:57 to a nucleotide sequence corresponding to the 3′ end of SEQ ID NO:57, but excluding the poly(A) tail at the 3′ end of SEQ ID NO:57. Also preferably the polynucleotide isolated according to the above process comprises a nucleotide sequence corresponding to the cDNA sequence of SEQ ID NO:57 from nucleotide 507 to nucleotide 722, and extending contiguously from a nucleotide sequence corresponding to the 5′ end of said sequence of SEQ ID NO:57 from nucleotide 507 to nucleotide 722, to a nucleotide sequence corresponding to the 3′ end of said sequence of SEQ ID NO:57 from nucleotide 507 to nucleotide 722. Also preferably the polynucleotide isolated according to the above process comprises a nucleotide sequence corresponding to the cDNA sequence of SEQ ID NO:57 from nucleotide 615 to nucleotide 722, and extending contiguously from a nucleotide sequence corresponding to the 5′ end of said sequence of SEQ ID NO:57 from nucleotide 615 to nucleotide 722, to a nucleotide sequence corresponding to the 3′ end of said sequence of SEQ ID NO:57 from nucleotide 615 to nucleotide 722.

In other embodiments, the present invention provides a composition comprising a protein, wherein said protein comprises an amino acid sequence selected from the group consisting of:

(a) the amino acid sequence of SEQ ID NO:58;

(b) fragments of the amino acid sequence of SEQ ID NO:58, each fragment comprising eight consecutive amino acids of SEQ ID NO:58; and

(c) the amino acid sequence encoded by the cDNA insert of clone na1010_{—}1 deposited with the ATCC under accession number 98623;

the protein being substantially free from other mammalian proteins. Preferably such protein comprises the amino acid sequence of SEQ ID NO:58. In further preferred embodiments, the present invention provides a protein comprising a fragment of the amino acid sequence of SEQ ID NO:58 having biological activity, the fragment preferably comprising eight (more preferably twenty, most preferably thirty) consecutive amino acids of SEQ ID NO:58, or a protein comprising a fragment of the amino acid sequence of SEQ ID NO:58 having biological activity, the fragment comprising the amino acid sequence from amino acid 31 to amino acid 40 of SEQ ID NO:58.

In one embodiment, the present invention provides a composition comprising an isolated polynucleotide selected from the group consisting of:

(a) a polynucleotide comprising the nucleotide sequence of SEQ ID NO:59;

(b) a polynucleotide comprising the nucleotide sequence of SEQ ID NO:59 from nucleotide 673 to nucleotide 987;

(c) a polynucleotide comprising the nucleotide sequence of SEQ ID NO:59 from nucleotide 868 to nucleotide 987;

(d) a polynucleotide comprising the nucleotide sequence of the full-length protein coding sequence of clone nf87_{—}1 deposited with the ATCC under accession number 98623;

(e) a polynucleotide encoding the full-length protein encoded by the cDNA insert of clone nf87_{—}1 deposited with the ATCC under accession number 98623;

(f) a polynucleotide comprising the nucleotide sequence of a mature protein coding sequence of clone nf87_{—}1 deposited with the ATCC under accession number 98623;

(g) a polynucleotide encoding a mature protein encoded by the cDNA insert of clone nf87_{—}1 deposited with the ATCC under accession number 98623;

(h) a polynucleotide encoding a protein comprising the amino acid sequence of SEQ ID NO:60;

(i) a polynucleotide encoding a protein comprising a fragment of the amino acid sequence of SEQ ID NO:60 having biological activity, the fragment comprising eight consecutive amino acids of SEQ ID NO:60;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:59 from nucleotide 673 to nucleotide 987; the nucleotide sequence of SEQ ID NO:59 from nucleotide 868 to nucleotide 987; the nucleotide sequence of the full-length protein coding sequence of clone nf87_{—}1 deposited with the ATCC under accession number 98623; or the nucleotide sequence of a mature protein coding sequence of clone nf87_{—}1 deposited with the ATCC under accession number 98623. In other preferred embodiments, the polynucleotide encodes the full-length or a mature protein encoded by the cDNA insert of clone nf87_{—}1 deposited with the ATCC under accession number 98623. In further preferred embodiments, the present invention provides a polynucleotide encoding a protein comprising a fragment of the amino acid sequence of SEQ ID NO:60 having biological activity, the fragment preferably comprising eight (more preferably twenty, most preferably thirty) consecutive amino acids of SEQ ID NO:60, or a polynucleotide encoding a protein comprising a fragment of the amino acid sequence of SEQ ID NO:60 having biological activity, the fragment comprising the amino acid sequence from amino acid 47 to amino acid 56 of SEQ ID NO:60.

Other embodiments provide the gene corresponding to the cDNA sequence of SEQ ID NO:59.

Further embodiments of the invention provide isolated polynucleotides produced according to a process selected from the group consisting of:

(a) a process comprising the steps of:

(i) preparing one ormore polynucleotideprobes thathybridize in 6×SSC at 65 degrees C to a nucleotide sequence selected from the group consisting of:

(aa) SEQ ID NO:59, but excluding the poly(A) tail at the 3′ end of SEQ ID NO:59; and

(ab) the nucleotide sequence of the cDNA insert of clone nf87_{—}1 deposited with the ATCC under accession number 98623;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:59, but excluding the poly(A) tail at the 3′ end of SEQ ID NO:59; and

(bb) the nucleotide sequence of the cDNA insert of clone nf87_{—}1 deposited with the ATCC under accession number 98623;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:59, and extending contiguously from a nucleotide sequence corresponding to the 5′ end of SEQ ID NO:59 to a nucleotide sequence corresponding to the 3′ end of SEQ ID NO:59 , but excluding the poly(A) tail at the 3′ end of SEQ ID NO:59. Also preferably the polynucleotide isolated according to the above process comprises a nucleotide sequence corresponding to the cDNA sequence of SEQ ID NO:59 from nucleotide 673 to nucleotide 987, and extending contiguously from a nucleotide sequence corresponding to the 5′ end of said sequence of SEQ ID NO:59 from nucleotide 673 to nucleotide 987, to a nucleotide sequence corresponding to the 3′ end of said sequence of SEQ ID NO:59 from nucleotide 673 to nucleotide 987. Also preferably the polynucleotide isolated according to the above process comprises a nucleotide sequence corresponding to the cDNA sequence of SEQ ID NO:59 from nucleotide 868 to nucleotide 987, and extending contiguously from a nucleotide sequence corresponding to the 5′ end of said sequence of SEQ ID NO:59 from nucleotide 868 to nucleotide 987, to a nucleotide sequence corresponding to the 3′ end of said sequence of SEQ ID NO:59 from nucleotide 868 to nucleotide 987.

In other embodiments, the present invention provides a composition comprising a protein, wherein said protein comprises an amino acid sequence selected from the group consisting of:

(a) the amino acid sequence of SEQ ID NO:60;

(b) fragments of the amino acid sequence of SEQ ID NO:60, each fragment comprising eight consecutive amino acids of SEQ ID NO:60; and

(c) the amino acid sequence encoded by the cDNA insert of clone nf87_{—}1 deposited with the ATCC under accession number 98623;

the protein being substantially free from other mammalian proteins. Preferably such protein comprises the amino acid sequence of SEQ ID NO:60. In further preferred embodiments, the present invention provides a protein comprising a fragment of the amino acid sequence of SEQ ID NO:60 having biological activity, the fragment preferably comprising eight (more preferably twenty, most preferably thirty) consecutive amino acids of SEQ ID NO:60, or a protein comprising a fragment of the amino acid sequence of SEQ ID NO:60 having biological activity, the fragment comprising the amino acid sequence from amino acid 47 to amino acid 56 of SEQ ID NO:60.

In one embodiment, the present invention provides a composition comprising an isolated polynucleotide selected from the group consisting of:

(a) a polynucleotide comprising the nucleotide sequence of SEQ ID NO:61;

(b) a polynucleotide comprising the nucleotide sequence of SEQ ID NO:61 from nucleotide 57 to nucleotide 824;

(c) a polynucleotide comprising the nucleotide sequence of SEQ ID NO:61 from nucleotide 114 to nucleotide 824;

(d) a polynucleotide comprising the nucleotide sequence of the full-length protein coding sequence of clone nh796_{—}1 deposited with the ATCC under accession number 98623;

(e) a polynucleotide encoding the full-length protein encoded by the cDNA insert of clone nh796_{—}1 deposited with the ATCC under accession number 98623;

(i) a polynucleotide comprising the nucleotide sequence of a mature protein coding sequence of clone nh796_{—}1 deposited with the ATCC under accession number 98623;

(g) a polynucleotide encoding a mature protein encoded by the cDNA insert of clone nh796_{—}1 deposited with the ATCC under accession number 98623;

(h) a polynucleotide encoding a protein comprising the amino acid sequence of SEQ ID NO:62;

(i) a polynucleotide encoding a protein comprising a fragment of the amino acid sequence of SEQ ID NO:62 having biological activity, the fragment comprising eight consecutive amino acids of SEQ ID NO:62;

(j) a polynucleotide which is an allelic variant of a polynucleotide of (a)-(g) above;

(k) a polynucleotide which encodes a species homologue of the protein of (h) or (i) above; and

(1) a polynucleotide that hybridizes under stringent conditions to any one of the polynucleotides specified in (a)-(i).

Preferably, such polynucleotide comprises the nucleotide sequence of SEQ ID NO:61 from nucleotide 57 to nucleotide 824; the nucleotide sequence of SEQ ID NO:61 from nucleotide 114 to nucleotide 824; the nucleotide sequence of the full-length protein coding sequence of clone nh796_{—}1 deposited with the ATCC under accession number 98623; or the nucleotide sequence of a mature protein coding sequence of clone nh796_{—}1 deposited with the ATCC under accession number 98623. In other preferred embodiments, the polynucleotide encodes the full-length or a mature protein encoded by the cDNA insert of clone nh796_{—}1 deposited with the ATCC under accession number 98623. In further preferred embodiments, the present invention provides a polynucleotide encoding a protein comprising a fragment of the amino acid sequence of SEQ ID NO:62 having biological activity, the fragment preferably comprising eight (more preferably twenty, most preferably thirty) consecutive amino acids of SEQ ID NO:62, or a polynucleotide encoding a protein comprising a fragment of the amino acid sequence of SEQ ID NO:62 having biological activity, the fragment comprising the amino acid sequence from amino acid 123 to amino acid 132 of SEQ ID NO:62.

Other embodiments provide the gene corresponding to the cDNA sequence of SEQ ID NO:61.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:61, but excluding the poly(A) tail at the 3′ end of SEQ ID NO:61; and

(ab) the nucleotide sequence of the cDNA insert of clone nh796_{—}1 deposited with the ATCC under accession number 98623;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:61, but excluding the poly(A) tail at the 3′ end of SEQ ID NO:61; and

(bb) the nucleotide sequence of the cDNA insert of clone nh796_{—}1 deposited with the ATCC under accession number 98623;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polvnucleotide isolated according to the above process comprises a nucleotide sequence corresponding to the cDNA sequence of SEQ ID NO:61, and extending contiguously from a nucleotide sequence corresponding to the 5′ end of SEQ ID NO:61 to a nucleotide sequence corresponding to the 3′ end of SEQ ID NO:61, but excluding the poly(A) tail at the 3′ end of SEQ ID NO:61. Also preferably the polynucleotide isolated according to the above process comprises a nucleotide sequence corresponding to the cDNA sequence of SEQ ID NO:61 from nucleotide 57 to nucleotide 824, and extending contiguously from a nucleotide sequence corresponding to the 5′ end of said sequence of SEQ ID NO:61 from nucleotide 57 to nucleotide 824, to a nucleotide sequence corresponding to the 3′ end of said sequence of SEQ ID NO:61 from nucleotide 57 to nucleotide 824. Also preferably the polynucleotide isolated according to the above process comprises a nucleotide sequence corresponding to the cDNA sequence of SEQ ID NO:61 from nucleotide 114 to nucleotide 824, and extending contiguously from a nucleotide sequence corresponding to the 5′ end of said sequence of SEQ ID NO:61 from nucleotide 114 to nucleotide 824, to a nucleotide sequence corresponding to the 3′ end of said sequence of SEQ ID NO:61 from nucleotide 114 to nucleotide 824.

In other embodiments, the present invention provides a composition comprising a protein, wherein said protein comprises an amino acid sequence selected from the group consisting of:

(a) the amino acid sequence of SEQ ID NO:62;

(b) fragments of the amino acid sequence of SEQ ID NO:62, each fragment comprising eight consecutive amino acids of SEQ ID NO:62; and

(c) the amino acid sequence encoded by the cDNA insert of clone nh796_{—}1 deposited with the ATCC under accession number 98623;

the protein being substantially free from other mammalian proteins. Preferably such protein comprises the amino acid sequence of SEQ ID NO:62. In further preferred embodiments, the present invention provides a protein comprising a fragment of the amino acid sequence of SEQ ID NO:62 having biological activity, the fragment preferably comprising eight (more preferably twenty, most preferably thirty) consecutive amino acids of SEQ ID NO:62, or a protein comprising a fragment of the amino acid sequence of SEQ ID NO:62 having biological activity, the fragment comprising the amino acid sequence from amino acid 123 to amino acid 132 of SEQ ID NO:62.

In one embodiment, the present invention provides a composition comprising an isolated polynucleotide selected from the group consisting of:

(a) a polynucleotide comprising the nucleotide sequence of SEQ ID NO:63;

(b) a polynucleotide comprising the nucleotide sequence of SEQ ID NO:63 from nucleotide 297 to nucleotide 542;

(c) a polynucleotide comprising the nucleotide sequence of SEQ ID NO:63 from nucleotide 510 to nucleotide 542;

(d) a polynucleotide comprising the nucleotide sequence of the full-length protein coding sequence of clone nn229_{—}1 deposited with the ATCC under accession number 98623;

(e) a polynucleotide encoding the full-length protein encoded by the cDNA insert of clone nn229_{—}1 deposited with the ATCC under accession number 98623;

(f) a polynucleotide comprising the nucleotide sequence of a mature protein coding sequence of clone nn229_{—}1 deposited with the ATCC under accession number 98623;

(g) a polynucleotide encoding a mature protein encoded by the cDNA insert of clone nn229_{—}1 deposited with the ATCC under accession number 98623;

(h) a polynucleotide encoding a protein comprising the amino acid sequence of SEQ ID NO:64;

(i) a polynucleotide encoding a protein comprising a fragment of the amino acid sequence of SEQ ID NO:64 having biological activity, the fragment comprising eight consecutive amino acids of SEQ ID NO:64;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:63 from nucleotide 297 to nucleotide 542; the nucleotide sequence of SEQ ID NO:63 from nucleotide 510 to nucleotide 542; the nucleotide sequence of the full-length protein coding sequence of clone nn229_{—}1 deposited with the ATCC under accession number 98623; or the nucleotide sequence of a mature protein coding sequence of clone nn229_{—}1 deposited with the ATCC under accession number 98623. In other preferred embodiments, the polynucleotide encodes the full-length or a mature protein encoded by the cDNA insert of clone nn229_{—}1 deposited with the ATCC under accession number 98623. In further preferred embodiments, the present invention provides a polynucleotide encoding a protein comprising a fragment of the amino acid sequence of SEQ ID NO:64 having biological activity, the fragment preferably comprising eight (more preferably twenty, most preferably thirty) consecutive amino acids of SEQ ID NO:64, or a polynucleotide encoding a protein comprising a fragment of the amino acid sequence of SEQ ID NO:64 having biological activity, the fragment comprising the amino acid sequence from amino acid 36 to amino acid 45 of SEQ ID NO:64.

Other embodiments provide the gene corresponding to the cDNA sequence of SEQ ID NO:63.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:63, but excluding the poly(A) tail at the 3′ end of SEQ ID NO:63; and

(ab) the nucleotide sequence of the cDNA insert of clone nn229_{—}1 deposited with the ATCC under accession number 98623;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:63, but excluding the poly(A) tail at the 3′ end of SEQ ID NO:63; and

(bb) the nucleotide sequence of the cDNA insert of clone nn229_{—}1 deposited with the ATCC under accession number 98623;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:63, and extending contiguously from a nucleotide sequence corresponding to the 5′ end of SEQ ID NO:63 to a nucleotide sequence corresponding to the 3′ end of SEQ ID NO:63, but excluding the poly(A) tail at the 3′ end of SEQ ID NO:63. Also preferably the polynucleotide isolated according to the above process comprises a nucleotide sequence corresponding to the cDNA sequence of SEQ ID NO:63 from nucleotide 297 to nucleotide 542, and extending contiguously from a nucleotide sequence corresponding to the 5′ end of said sequence of SEQ ID NO:63 from nucleotide 297 to nucleotide 542, to a nucleotide sequence corresponding to the 3′ end of said sequence of SEQ ID NO:63 from nucleotide 297 to nucleotide 542. Also preferably the polynucleotide isolated according to the above process comprises a nucleotide sequence corresponding to the cDNA sequence of SEQ ID NO:63 from nucleotide 510 to nucleotide 542, and extending contiguously from a nucleotide sequence corresponding to the 5′ end of said sequence of SEQ ID NO:63 from nucleotide 510 to nucleotide 542, to a nucleotide sequence corresponding to the 3′ end of said sequence of SEQ ID NO:63 from nucleotide 510 to nucleotide 542.

In other embodiments, the present invention provides a composition comprising a protein, wherein said protein comprises an amino acid sequence selected from the group consisting of:

(a) the amino acid sequence of SEQ ID NO:64;

(b) fragments of the amino acid sequence of SEQ ID NO:64, each fragment comprising eight consecutive amino acids of SEQ ID NO:64; and

(c) the amino acid sequence encoded by the cDNA insert of clone nn229_{—}1 deposited with the ATCC under accession number 98623;

the protein being substantially free from other mammalian proteins. Preferably such protein comprises the amino acid sequence of SEQ ID NO:64. In further preferred embodiments, the present invention provides a protein comprising a fragment of the amino acid sequence of SEQ ID NO:64 having biological activity, the fragment preferably comprising eight (more preferably twenty, most preferably thirty) consecutive amino acids of SEQ ID NO:64, or a protein comprising a fragment of the amino acid sequence of SEQ ID NO:64 having biological activity, the fragment comprising the amino acid sequence from amino acid 36 to amino acid 45 of SEQ ID NO:64.

In one embodiment, the present invention provides a composition comprising an isolated polynucleotide selected from the group consisting of:

(a) a polynucleotide comprising the nucleotide sequence of SEQ ID NO:65;

(b) a polynucleotide comprising the nucleotide sequence of SEQ ID NO:65 from nucleotide 547 to nucleotide 750;

(c) a polynucleotide comprising the nucleotide sequence of SEQ ID NO:65 from nucleotide 601 to nucleotide 750;

(d) a polynucleotide comprising the nucleotide sequence of the full-length protein coding sequence of clone np156_{—}1 deposited with the ATCC under accession number 98623;

(e) a polynucleotide encoding the full-length protein encoded by the cDNA insert of clone np156_{—}1 deposited with the ATCC under accession number 98623;

(f) a polynucleotide comprising the nucleotide sequence of a mature protein coding sequence of clone np156_{—}1 deposited with the ATCC under accession number 98623;

(g) a polynucleotide encoding a mature protein encoded by the cDNA insert of clone np156_{—}1 deposited with the ATCC under accession number 98623;

(h) a polynucleotide encoding a protein comprising the amino acid sequence of SEQ ID NO:66;

(i) a polynucleotide encoding a protein comprising a fragment of the amino acid sequence of SEQ ID NO:66 having biological activity, the fragment comprising eight consecutive amino acids of SEQ ID NO:66;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:65 from nucleotide 547 to nucleotide 750; the nucleotide sequence of SEQ ID NO:65 from nucleotide 601 to nucleotide 750; the nucleotide sequence of the full-length protein coding sequence of clone np156_{—}1 deposited with the ATCC under accession number 98623; or the nucleotide sequence of a mature protein coding sequence of clone np156_{—}1 deposited with the ATCC under accession number 98623. In other preferred embodiments, the polynucleotide encodes the full-length or a mature protein encoded by the cDNA insert of clone np156_{—}1 deposited with the ATCC under accession number 98623. In further preferred embodiments, the present invention provides a polynucleotide encoding a protein comprising a fragment of the amino acid sequence of SEQ ID NO:66 having biological activity, the fragment preferably comprising eight (more preferably twenty, most preferably thirty) consecutive amino acids of SEQ ID NO:66, or a polynucleotide encoding a protein comprising a fragment of the amino acid sequence of SEQ ID NO:66 having biological activity, the fragment comprising the amino acid sequence from amino acid 29 to amino acid 38 of SEQ ID NO:66.

Other embodiments provide the gene corresponding to the cDNA sequence of SEQ ID NO:65.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:65, but excluding the poly(A) tail at the 3′ end of SEQ ID NO:65; and

(ab) the nucleotide sequence of the cDNA insert of clone np156_{—}1 deposited with the ATCC under accession number 98623;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:65, but excluding the poly(A) tail at the 3′ end of SEQ ID NO:65; and

(bb) the nucleotide sequence of the cDNA insert of clone np156_{—}1 deposited with the ATCC under accession number 98623;

(ii) hybridizing said primer(s) to human genomic DNA in conditions at least as stringent as 4(SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:65, and extending contiguously from a nucleotide sequence corresponding to the 5′ end of SEQ ID NO:65 to a nucleotide sequence corresponding to the 3′ end of SEQ ID NO:65, but excluding the poly(A) tail at the 3′ end of SEQ ID NO:65. Also preferably the polynucleotide isolated according to the above process comprises a nucleotide sequence corresponding to the cDNA sequence of SEQ ID NO:65 from nucleotide 547 to nucleotide 750, and extending contiguously from a nucleotide sequence corresponding to the 5′ end of said sequence of SEQ ID NO:65 from nucleotide 547 to nucleotide 750, to a nucleotide sequence corresponding to the 3′ end of said sequence of SEQ ID NO:65 from nucleotide 547 to nucleotide 750. Also preferably the polynucleotide isolated according to the above process comprises a nucleotide sequence corresponding to the cDNA sequence of SEQ ID NO:65 from nucleotide 601 to nucleotide 750, and extending contiguously from a nucleotide sequence corresponding to the 5′ end of said sequence of SEQ ID NO:65 from nucleotide 601 to nucleotide 750, to a nucleotide sequence corresponding to the 3′ end of said sequence of SEQ ID NO:65 from nucleotide 601 to nucleotide 750.

In other embodiments, the present invention provides a composition comprising a protein, wherein said protein comprises an amino acid sequence selected from the group consisting of:

(a) the amino acid sequence of SEQ ID NO:66;

(b) fragments of the amino acid sequence of SEQ ID NO:66, each fragment comprising eight consecutive amino acids of SEQ ID NO:66; and

(c) the amino acid sequence encoded by the cDNA insert of clone np156_{—}1 deposited with the ATCC under accession number 98623;

the protein being substantially free from other mammalian proteins. Preferably such protein comprises the amino acid sequence of SEQ ID NO:66. In further preferred embodiments, the present invention provides a protein comprising a fragment of the amino acid sequence of SEQ ID NO:66 having biological activity, the fragment preferably comprising eight (more preferably twenty, most preferably thirty) consecutive amino acids of SEQ ID NO:66, or a protein comprising a fragment of the amino acid sequence of SEQ ID NO:66 having biological activity, the fragment comprising the amino acid sequence from amino acid 29 to amino acid 38 of SEQ ID NO:66.

In one embodiment, the present invention provides a composition comprising an isolated polynucleotide selected from the group consisting of:

(a) a polynucleotide comprising the nucleotide sequence of SEQ ID NO:67;

(b) a polynucleotide comprising the nucleotide sequence of SEQ ID NO:67 from nucleotide 310 to nucleotide 459;

(c) a polynucleotide comprising the nucleotide sequence of SEQ ID NO:67 from nucleotide 445 to nucleotide 459;

(d) a polynucleotide comprising the nucleotide sequence of the full-length protein coding sequence of clone bg570_{—}1 deposited with the ATCC under accession number 98629;

(e) a polynucleotide encoding the full-length protein encoded by the cDNA insert of clone bg570_{—}1 deposited with the ATCC under accession number 98629;

(f) a polynucleotide comprising the nucleotide sequence of a mature protein coding sequence of clone bg570_{—}1 deposited with the ATCC under accession number 98629;

(g) a polynucleotide encoding a mature protein encoded by the cDNA insert of clone bg570_{—}1 deposited with the ATCC under accession number 98629;

(h) a polynucleotide encoding a protein comprising the amino acid sequence of SEQ ID NO:68;

(i) a polynucleotide encoding a protein comprising a fragment of the amino acid sequence of SEQ ID NO:68 having biological activity, the fragment comprising eight consecutive amino acids of SEQ ID NO:68;

(j) a polynucleotide which is an allelic variant of a polynudeotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:67 from nucleotide 310 to nucleotide 459; the nucleotide sequence of SEQ ID NO:67 from nucleotide 445 to nucleotide 459; the nucleotide sequence of the full-length protein coding sequence of clone bg570_{—}1 deposited with the ATCC under accession number 98629; or the nucleotide sequence of a mature protein coding sequence of clone bg570_{—}1 deposited with the ATCC under accession number 98629. In other preferred embodiments, the polynucleotide encodes the full-length or a mature protein encoded by the cDNA insert of clone bg570_{—}1 deposited with the ATCC under accession number 98629. In further preferred embodiments, the present invention provides a polynucleotide encoding a protein comprising a fragment of the amino acid sequence of SEQ ID NO:68 having biological activity, the fragment preferably comprising eight (more preferably twenty, most preferably thirty) consecutive amino acids of SEQ ID NO:68, or a polynucleotide encoding a protein comprising a fragment of the amino acid sequence of SEQ ID NO:68 having biological activity, the fragment comprising the amino acid sequence from amino acid 20 to amino acid 29 of SEQ ID NO:68.

Other embodiments provide the gene corresponding to the cDNA sequence of SEQ ID NO:67.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:67, but excluding the poly(A) tail at the 3′ end of SEQ ID NO:67; and

(ab) the nucleotide sequence of the cDNA insert of clone bg570_{—}1 deposited with the ATCC under accession number 98629;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:67, but excluding the poly(A) tail at the 3′ end of SEQ ID NO:67; and

(bb) the nucleotide sequence of the cDNA insert of clone bg570_{—}1 deposited with the ATCC under accession number 98629;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:67, and extending contiguously from a nucleotide sequence corresponding to the 5′ end of SEQ ID NO:67 to a nucleotide sequence corresponding to the 3′ end of SEQ ID NQ:67, but excluding the poly(A) tail at the 3′ end of SEQ ID NO:67. Also preferably the polynucleotide isolated according to the above process comprises a nucleotide sequence corresponding to the cDNA sequence of SEQ ID NO:67 from nucleotide 310 to nucleotide 459, and extending contiguously from a nucleotide sequence corresponding to the 5′ end of said sequence of SEQ ID NO:67 from nucleotide 310 to nucleotide 459, to a nucleotide sequence corresponding to the 3′ end of said sequence of SEQ ID NO:67 from nucleotide 310 to nucleotide 459. Also preferably the polynucleotide isolated according to the above process comprises a nucleotide sequence corresponding to the cDNA sequence of SEQ ID NO:67 from nucleotide 445 to nucleotide 459, and extending contiguously from a nucleotide sequence corresponding to the 5′ end of said sequence of SEQ ID NO:67 from nucleotide 445 to nucleotide 459, to a nucleotide sequence corresponding to the 3′ end of said sequence of SEQ ID NO:67 from nucleotide 445 to nucleotide 459.

In other embodiments, the present invention provides a composition comprising a protein, wherein said protein comprises an amnino acid sequence selected from the group consisting of:

(a) the amino acid sequence of SEQ ID NO:68;

(b) fragments of the amino acid sequence of SEQ ID NO:68, each fragment comprising eight consecutive amino acids of SEQ ID NO:68; and

(c) the amino acid sequence encoded by the cDNA insert of clone bg570_{—}1 deposited with the ATCC under accession number 98629;

the protein being substantially free from other mammalian proteins. Preferably such protein comprises the amino acid sequence of SEQ ID NO:68. In further preferred embodiments, the present invention provides a protein comprising a fragment of the amino acid sequence of SEQ ID NO:68 having biological activity, the fragment preferably comprising eight (more preferably twenty, most preferably thirty) consecutive amino acids of SEQ ID NO:68, or a protein comprising a fragment of the amino acid sequence of SEQ ID NO:68 having biological activity, the fragment comprising the amino acid sequence from amino acid 20 to amino acid 29 of SEQ ID NO:68.

In one embodiment, the present invention provides a composition comprising an isolated polynucleotide selected from the group consisting of:

(a) a polynucleotide comprising the nucleotide sequence of SEQ ID NO:69;

(b) a polynucleotide comprising the nucleotide sequence of SEQ ID NO:69 from nucleotide 90 to nucleotide 1019;

(c) a polynucleotide comprising the nucleotide sequence of SEQ ID NO:69 from nucleotide 243 to nucleotide 1019;

(d) a polynucleotide comprising the nucleotide sequence of the full-length protein coding sequence of clone bi120_{—}2 deposited with the ATCC under accession number 98629;

(e) a polynucleotide encoding the full-length protein encoded by the cDNA insert of clone bi120_{—}2 deposited with the ATCC under accession number 98629;

(f) a polynucleotide comprising the nucleotide sequence of a mature protein coding sequence of clone bi120_{—}2 deposited with the ATCC under accession number 98629;

(g) a polynucleotide encoding a mature protein encoded by the cDNA insert of clone bi120_{—}2 deposited with the ATCC under accession number 98629;

(h) a polynucleotide encoding a protein comprising the amino acid sequence of SEQ ID NO:70;

(i) a polynucleotide encoding a protein comprising a fragment of the amino acid sequence of SEQ ID NO:70 having biological activity, the fragment comprising eight consecutive amino acids of SEQ ID NO:70;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:69 from nucleotide 90 to nucleotide 1019; the nucleotide sequence of SEQ ID NO:69 from nucleotide 243 to nucleotide 1019; the nucleotide sequence of the full-length protein coding sequence of clone bi120_{—}2 deposited with the ATCC under accession number 98629; or the nucleotide sequence of a mature protein coding sequence of clone bi120_{—}2 deposited with the ATCC under accession number 98629. In other preferred embodiments, the polynucleotide encodes the full-length or a mature protein encoded by the cDNA insert of clone bi120_{—}2 deposited with the ATCC under accession number 98629. In further preferred embodiments, the present invention provides a polynucleotide encoding a protein comprising a fragment of the amino acid sequence of SEQ ID NO:70 having biological activity, the fragment preferably comprising eight (more preferably twenty, most preferably thirty) consecutive amino acids of SEQ ID NO:70, or a polynucleotide encoding a protein comprising a fragment of the amino acid sequence of SEQ ID NO:70 having biological activity, the fragment comprising the amino acid sequence from amino acid 149 to amino acid 158 of SEQ ID NO:70.

Other embodiments provide the gene corresponding to the cDNA sequence of SEQ ID NO:69.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:69, but excluding the poly(A) tail at the 3′ end of SEQ ID NO:69; and

(ab) the nucleotide sequence of the cDNA insert of clone bi120_{—}2 deposited with the ATCC under accession number 98629;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:69, but excluding the poly(A) tail at the 3′ end of SEQ ID NO:69; and

(bb) the nucleotide sequence of the cDNA insert of clone bi120_{—}2 deposited with the ATCC under accession number 98629;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:69, and extending contiguously from a nucleotide sequence corresponding to the 5′ end of SEQ ID NO:69 to a nucleotide sequence corresponding to the 3′ end of SEQ ID NO:69, but excluding the poly(A) tail at the 3′ end of SEQ ID NO:69. Also preferably the polynucleotide isolated according to the above process comprises a nucleotide sequence corresponding to the cDNA sequence of SEQ ID NO:69 from nucleotide 90 to nucleotide 1019, and extending contiguously from a nucleotide sequence corresponding to the 5′ end of said sequence of SEQ ID NO:69 from nucleotide 90 to nucleotide 1019, to a nucleotide sequence corresponding to the 3′ end of said sequence of SEQ ID NO:69 from nucleotide 90 to nucleotide 1019. Also preferably the polynucleotide isolated according to the above process comprises a nucleotide sequence corresponding to the cDNA sequence of SEQ ID NO:69 from nucleotide 243 to nucleotide 1019, and extending contiguously from a nucleotide sequence corresponding to the 5′ end of said sequence of SEQ ID NO:69 from nucleotide 243 to nucleotide 1019, to a nucleotide sequence corresponding to the 3′ end of said sequence of SEQ ID NO:69 from nucleotide 243 to nucleotide 1019.

In other embodiments, the present invention provides a composition comprising a protein, wherein said protein comprises an amino acid sequence selected from the group consisting of:

(a) the amino acid sequence of SEQ ID NO:70;

(b) fragments of the amino acid sequence of SEQ ID NO:70, each fragment comprising eight consecutive amino acids of SEQ ID NO:70; and

(c) the amino acid sequence encoded by the cDNA insert of clone bi120_{—}2 deposited with the ATCC under accession number 98629;

the protein being substantially free from other mammalian proteins. Preferably such protein comprises the amino acid sequence of SEQ ID NO:70. In further preferred embodiments, the present invention provides a protein comprising a fragment of the amino acid sequence of SEQ ID NO:70 having biological activity, the fragment preferably comprising eight (more preferably twenty, most preferably thirty) consecutive amino acids of SEQ ID NO:70, or a protein comprising a fragment of the amino acid sequence of SEQ ID NO:70 having biological activity, the fragment comprising the amino acid sequence from amino acid 149 to amino acid 158 of SEQ ID NO:70.

In one embodiment, the present invention provides a composition comprising an isolated polynucleotide selected from the group consisting of:

(a) a polynucleotide comprising the nucleotide sequence of SEQ ID NO:71;

(b) a polynucleotide comprising the nucleotide sequence of SEQ ID NO:71 from nucleotide 682 to nucleotide 894;

(c) a polynucleotide comprising the nucleotide sequence of the full-length protein coding sequence of clone bn594_{—}1 deposited with the ATCC under accession number 98629;

(d) a polynucleotide encoding the full-length protein encoded by the cDNA insert of clone bn594_{—}1 deposited with the ATCC under accession number 98629;

(e) a polynucleotide comprising the nucleotide sequence of a mature protein coding sequence of clone bn594_{—}1 deposited with the ATCC under accession number 98629;

(f) a polynucleotide encoding a mature protein encoded by the cDNA insert of clone bn594_{—}1 deposited with the ATCC under accession number 98629;

(g) a polynucleotide encoding a protein comprising the amino acid sequence of SEQ ID NO:72;

(h) a polynucleotide encoding a protein comprising a fragment of the amino acid sequence of SEQ ID NO:72 having biological activity, the fragment comprising eight consecutive amino acids of SEQ ID NO:72;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:71 from nucleotide 682 to nucleotide 894; the nucleotide sequence of the full-length protein coding sequence of clone bn594_{—}1 deposited with the ATCC under accession number 98629; or the nucleotide sequence of a mature protein coding sequence of clone bn594_{—}1 deposited with the ATCC under accession number 98629. In other preferred embodiments, the polynucleotide encodes the full-length or a mature protein encoded by the cDNA insert of clone bn594_{—}1 deposited with the ATCC under accession number 98629. In further preferred embodiments, the present invention provides a polynucleotide encoding a protein comprising a fragment of the amino acid sequence of SEQ ID NO:72 having biological activity, the fragment preferably comprising eight (more preferably twenty, most preferably thirty) consecutive amino acids of SEQ ID NO:72, or a polynucleotide encoding a protein comprising a fragment of the amino acid sequence of SEQ ID NO:72 having biological activity, the fragment comprising the armino acid sequence from amino acid 30 to amino acid 39 of SEQ ID NO:72.

Other embodiments provide the gene corresponding to the cDNA sequence of SEQ ID NO:71.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:71, but excluding the poly(A) tail at the 3′ end of SEQ ID NO:71; and

(ab) the nucleotide sequence of the cDNA insert of clone bn594_{—}1 deposited with the ATCC under accession number 98629;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:71, but excluding the poly(A) tail at the 3′ end of SEQ ID NO:71; and

(bb) the nucleotide sequence of the cDNA insert of clone bn594_{—}1 deposited with the ATCC under accession number 98629;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:71, and extending contiguously from a nucleotide sequence corresponding to the 5′ end of SEQ ID NO:71 to a nucleotide sequence corresponding to the 3′ end of SEQ ID NO:71 , but excluding the poly(A) tail at the 3′ end of SEQ ID NO:71. Also preferably the polynucleotide isolated according to the above process comprises a nucleotide sequence corresponding to the cDNA sequence of SEQ ID NO:71 from nucleotide 682 to nucleotide 894, and extending contiguously from a nucleotide sequence corresponding to the 5′ end of said sequence of SEQ ID NO:71 from nucleotide 682 to nucleotide 894, to a nucleotide sequence corresponding to the 3′ end of said sequence of SEQ ID NO:71 from nucleotide 682 to nucleotide 894.

In other embodiments, the present invention provides a composition comprising a protein, wherein said protein comprises an amino acid sequence selected from the group consisting of:

(a) the amino acid sequence of SEQ ID NO:72;

(b) fragments of the amino acid sequence of SEQ ID NO:72, each fragment comprising eight consecutive amino acids of SEQ ID NO:72; and

(c) the amino acid sequence encoded by the cDNA insert of clone bn594_{—}1 deposited with the ATCC under accession number 98629;

the protein being substantially free from other mammalian proteins. Preferably such protein comprises the amino acid sequence of SEQ ID NO:72. In further preferred embodiments, the present invention provides a protein comprising a fragment of the amino acid sequence of SEQ ID NO:72 having biological activity, the fragment preferably comprising eight (more preferably twenty, most preferably thirty) consecutive amino acids of SEQ ID NO:72, or a protein comprising a fragment of the amino acid sequence of SEQ ID NO:72 having biological activity, the fragment comprising the amino acid sequence from amino acid 30 to amino acid 39 of SEQ ID NO:72.

In one embodiment, the present invention provides a composition comprising an isolated polynucleotide selected from the group consisting of:

(a) a polynucleotide comprising the nucleotide sequence of SEQ ID NO:73;

(b) a polynucleotide comprising the nucleotide sequence of SEQ ID NO:73 from nucleotide 1184 to nucleotide 1582;

(c) a polynucleotide comprising the nucleotide sequence of SEQ ID NO:73 from nucleotide 1265 to nucleotide 1582;

(d) a polynucleotide comprising the nucleotide sequence of the full-length protein coding sequence of clone en554_{—}1 deposited with the ATCC under accession number 98629;

(e) a polynucleotide encoding the full-length protein encoded by the cDNA insert of clone en554_{—}1 deposited with the ATCC under accession number 98629;

(f) a polynucleotide comprising the nucleotide sequence of a mature protein coding sequence of clone en554_{—}1 deposited with the ATCC under accession number 98629;

(g) a polynucleotide encoding a mature protein encoded by the cDNA insert of clone en554_{—}1 deposited with the ATCC under accession number 98629;

(h) a polynucleotide encoding a protein comprising the amino acid sequence of SEQ ID NO:74;

(i) a polynucleotide encoding a protein comprising a fragment of the amino acid sequence of SEQ ID NO:74 having biological activity, the fragment comprising eight consecutive amino acids of SEQ ID NO:74;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:73 from nucleotide 1184 to nucleotide 1582; the nucleotide sequence of SEQ ID NO:73 from nucleotide 1265 to nucleotide 1582; the nucleotide sequence of the full-length protein coding sequence of clone en554_{—}1 deposited with the ATCC under accession number 98629; or the nucleotide sequence of a mature protein coding sequence of clone en554_{—}1 deposited with the ATCC under accession number 98629. In other preferred embodiments, the polynucleotide encodes the full-length or a mature protein encoded by the cDNA insert of clone en554_{—}1 deposited with the ATCC under accession number 98629. In further preferred embodiments, the present invention provides a polynucleotide encoding a protein comprising a fragment of the amino acid sequence of SEQ ID NO:74 having biological activity, the fragment preferably comprising eight (more preferably twenty, most preferably thirty) consecutive amino acids of SEQ ID NO:74, or a polynucleotide encoding a protein comprising a fragment of the amnino acid sequence of SEQ ID NO:74 having biological activity, the fragment comprising the amino acid sequence from amino acid 61 to amino acid 70 of SEQ ID NO:74.

Other embodiments provide the gene corresponding to the cDNA sequence of SEQ ID NO:73.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:73, but excluding the poly(A) tail at the 3′ end of SEQ ID NO:73; and

(ab) the nucleotide sequence of the cDNA insert of clone en554_{—}1 deposited with the ATCC under accession number 98629;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:73, but excluding the poly(A) tail at the 3′ end of SEQ ID NO:73; and

(bb) the nucleotide sequence of the cDNA insert of clone en554 1 deposited with the ATCC under accession number 98629;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:73, and extending contiguously from a nucleotide sequence corresponding to the 5′ end of SEQ ID NO:73 to a nucleotide sequence corresponding to the 3′ end of SEQ ID NO:73, but excluding the poly(A) tail at the 3′ end of SEQ ID NO:73. Also preferably the polynucleotide isolated according to the above process comprises a nucleotide sequence corresponding to the cDNA sequence of SEQ ID NO:73 from nucleotide 1184 to nucleotide 1582, and extending contiguously from a nucleotide sequence corresponding to the 5′ end of said sequence of SEQ ID NO:73 from nucleotide 1184 to nucleotide 1582, to a nucleotide sequence corresponding to the 3′ end of said sequence of SEQ ID NO:73 from nucleotide 1184 to nucleotide 1582. Also preferably the polynucleotide isolated according to the above process comprises a nucleotide sequence corresponding to the cDNA sequence of SEQ ID NO:73 from nucleotide 1265 to nucleotide 1582, and extending contiguously from a nucleotide sequence corresponding to the 5′ end of said sequence of SEQ ID NO:73 from nucleotide 1265 to nucleotide 1582, to a nucleotide sequence corresponding to the 3′ end of said sequence of SEQ ID NO:73 from nucleotide 1265 to nucleotide 1582.

In other embodiments, the present invention provides a composition comprising a protein, wherein said protein comprises an amino acid sequence selected from the group consisting of:

(a) the amino acid sequence of SEQ ID NO:74;

(b) fragments of the amino acid sequence of SEQ ID NO:74, each fragment comprising eight consecutive amino acids of SEQ ID NO:74; and

(c) the amino acid sequence encoded by the cDNA insert of clone en554_{—}1 deposited with the ATCC under accession number 98629;

the protein being substantially free from other mammalian proteins. Preferably such protein comprises the amino acid sequence of SEQ ID NO:74. In further preferred embodiments, the present invention provides a protein comprising a fragment of the amino acid sequence of SEQ ID NO:74 having biological activity, the fragment preferably comprising eight (more preferably twenty, most preferably thirty) consecutive amino acids of SEQ ID NO:74, or a protein comprising a fragment of the amino acid sequence of SEQ ID NO:74 having biological activity, the fragment comprising the amino acid sequence from amino acid 61 to amino acid 70 of SEQ ID NO:74.

In one embodiment, the present invention provides a composition comprising an isolated polvnucleotide selected from the group consisting of:

(a) a polynucleotide comprising the nucleotide sequence of SEQ ID NO:75;

(b) a polynucleotide comprising the nucleotide sequence of SEQ ID NO:75 from nucleotide 79 to nucleotide 504;

(c) a polynucleotide comprising the nucleotide sequence of SEQ ID NO:75 from nucleotide 322 to nucleotide 504;

(d) a polynucleotide comprising the nucleotide sequence of the full-length protein coding sequence of clone na474_{—}10 deposited with the ATCC under accession number 98629;

(e) a polynucleotide encoding the full-length protein encoded by the cDNA insert of clone na474_{—}10 deposited with the ATCC under accession number 98629;

(f) a polynucleotide comprising the nucleotide sequence of a mature protein coding sequence of clone na474_{—}10 deposited with the ATCC under accession number 98629;

(g) a polynucleotide encoding a mature protein encoded by the cDNA insert of clone na474_{—}10 deposited with the ATCC under accession number 98629;

(h) a polynucleotide encoding a protein comprising the amino acid sequence of SEQ ID NO:76;

(i) a polynucleotide encoding a protein comprising a fragment of the amino acid sequence of SEQ ID NO:76 having biological activity, the fragment comprising eight consecutive amino acids of SEQ ID NO:76;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:75 from nucleotide 79 to nucleotide 504; the nucleotide sequence of SEQ ID NO:75 from nucleotide 322 to nucleotide 504; the nucleotide sequence of the full-length protein coding sequence of clone na474_{—}10 deposited with the ATCC under accession number 98629; or the nucleotide sequence of a mature protein coding sequence of clone na474_{—}10 deposited with the ATCC under accession number 98629. In other preferred embodiments, the polynucleotide encodes the full-length or a mature protein encoded by the cDNA insert of clone na474_{—}10 deposited with the ATCC under accession number 98629. In further preferred embodiments, the present invention provides a polynucleotide encoding a protein comprising a fragment of the amino acid sequence of SEQ ID NO:76 having biological activity, the fragment preferably comprising eight (more preferably twenty, most preferably thirty) consecutive amino acids of SEQ ID NO:76, or a polynucleotide encoding a protein comprising a fragment of the amino acid sequence of SEQ ID NO:76 having biological activity, the fragment comprising the amino acid sequence from amino acid 66 to amino acid 75 of SEQ ID NO:76.

Other embodiments provide the gene corresponding to the cDNA sequence of SEQ ID NO:75.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:75, but excluding the poly(A) tail at the 3′ end of SEQ ID NO:75; and

(ab) the nucleotide sequence of the cDNA insert of clone na474_{—}10 deposited with the ATCC under accession number 98629;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:75, but excluding the poly(A) tail at the 3′ end of SEQ ID NO:75; and

(bb) the nucleotide sequence of the cDNA insert of clone na474_{—}10 deposited with the ATCC under accession number 98629;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:75, and extending contiguously from a nucleotide sequence corresponding to the 5′ end of SEQ ID NO:75 to a nucleotide sequence corresponding to the 3′ end of SEQ ID NO:75, but excluding the poly(A) tail at the 3′ end of SEQ ID NO:75. Also preferably the polynucleotide isolated according to the above process comprises a nucleotide sequence corresponding to the cDNA sequence of SEQ ID NO:75 from nucleotide 79 to nucleotide 504, and extending contiguously from a nucleotide sequence corresponding to the 5′ end of said sequence of SEQ ID NO:75 from nucleotide 79 to nucleotide 504, to a nucleotide sequence corresponding to the 3′ end of said sequence of SEQ ID NO:75 from nucleotide 79 to nucleotide 504. Also preferably the polynucleotide isolated according to the above process comprises a nucleotide sequence corresponding to the cDNA sequence of SEQ ID NO:75 from nucleotide 322 to nucleotide 504, and extending contiguously from a a-nucleotide sequence corresponding to the 5′ end of said sequence of SEQ ID NO:75 from nucleotide 322 to nucleotide 504, to a nucleotide sequence corresponding to the 3′ end of said sequence of SEQ ID NO:75 from nucleotide 322 to nucleotide 504.

In other embodiments, the present invention provides a composition comprising a protein, wherein said protein comprises an amino acid sequence selected from the group consisting of:

(a) the amino acid sequence of SEQ ID NO:76;

(b) fragments of the amino acid sequence of SEQ ID NO:76, each fragment comprising eight consecutive amino acids of SEQ ID NO:76; and

(c) the amino acid sequence encoded by the cDNA insert of clone na474_{—}10 deposited with the ATCC under accession number 98629;

the protein being substantially free from other mammalian proteins. Preferably such protein comprises the amino acid sequence of SEQ ID NO:76. In further preferred embodiments, the present invention provides a protein comprising a fragment of the amino acid sequence of SEQ ID NO:76 having biological activity, the fragment preferably comprising eight (more preferably twenty, most preferably thirty) consecutive amino acids of SEQ ID NO:76, or a protein comprising a fragment of the amino acid sequence of SEQ ID NO:76 having biological activity, the fragment comprising the amino acid sequence from amino acid 66 to amino acid 75 of SEQ ID NO:76.

In one embodiment, the present invention provides a composition comprising an isolated polynucleotide selected from the group consisting of:

(a) a polynucleotide comprising the nucleotide sequence of SEQ ID NO:77;

(b) a polynucleotide comprising the nucleotide sequence of SEQ ID NO:77 from nucleotide 92 to nucleotide 1435;

(c) a polynucleotide comprising the nucleotide sequence of SEQ ID NO:77 from nucleotide 170 to nucleotide 1435;

(d) a polynucleotide comprising the nucleotide sequence of the full-length protein coding sequence of clone nn16_{—}10 deposited with the ATCC under accession number 98629;

(e) a polynucleotide encoding the full-length protein encoded by the cDNA insert of clone nn16_{—}10 deposited with the ATCC under accession number 98629;

(f) a polynucleotide comprising the nucleotide sequence of a mature protein coding sequence of clone nn16_{—}10 deposited with the ATCC under accession number 98629;

(g) a polynucleotide encoding a mature protein encoded by the cDNA insert of clone nn16_{—}10 deposited with the ATCC under accession number 98629;

(h) a polynucleotide encoding a protein comprising the amino acid sequence of SEQ ID NO:78;

(i) a polynucleotide encoding a protein comprising a fragment of the amino acid sequence of SEQ ID NO:78 having biological activity, the fragment comprising eight consecutive amino acids of SEQ ID NO:78;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:77 from nucleotide 92 to nucleotide 1435; the nucleotide sequence of SEQ ID NO:77 from nucleotide 170 to nucleotide 1435; the nucleotide sequence of the full-length protein coding sequence of clone nn16_{—}10 deposited with the ATCC under accession number 98629; or the nucleotide sequence of a mature protein coding sequence of clone nn16_{—}10 deposited with the ATCC under accession number 98629. In other preferred embodiments, the polynucleotide encodes the full-length or a mature protein encoded by the cDNA insert of clone nn16_{—}10 deposited with the ATCC under accession number 98629. In further preferred embodiments, the present invention provides a polynucleotide encoding a protein comprising a fragment of the amino acid sequence of SEQ ID NO:78 having biological activity, the fragment preferably comprising eight (more preferably twenty, most preferably thirty) consecutive amino acids of SEQ ID NO:78, or a polynucleotide encoding a protein comprising a fragment of the amino acid sequence of SEQ ID NO:78 having biological activity, the fragment comprising the amino acid sequence from amino acid 218 to amino acid 227 of SEQ ID NO:78.

Other embodiments provide the gene corresponding to the cDNA sequence of SEQ ID NO:77.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:77, but excluding the poly(A) tail at the 3′ end of SEQ ID NO:77; and

(ab) the nucleotide sequence of the cDNA insert of clone nn16_{—}10 deposited with the ATCC under accession number 98629;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:77, but excluding the poly(A) tail at the 3′ end of SEQ ID NO:77; and

(bb) the nucleotide sequence of the cDNA insert of clone nn16_{—}10 deposited with the ATCC under accession number 98629;

(ii) hybridizing said primer(s) to human genornic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:77, and extending contiguously from a nucleotide sequence corresponding to the 5′ end of SEQ ID NO:77 to a nucleotide sequence corresponding to the 3′ end of SEQ ID NO:77, but excluding the poly(A) tail at the 3′ end of SEQ ID NO:77. Also preferably the polynucleotide isolated according to the above process comprises a nucleotide sequence corresponding to the cDNA sequence of SEQ ID NO:77 from nucleotide 92 to nucleotide 1435, and extending contiguously from a nucleotide sequence corresponding to the 5′ end of said sequence of SEQ ID NO:77 from nucleotide 92 to nucleotide 1435, to a nucleotide sequence corresponding to the 3′ end of said sequence of SEQ ID NO:77 from nucleotide 92 to nucleotide 1435. Also preferably the polynucleotide isolated according to the above process comprises a nucleotide sequence corresponding to the cDNA sequence of SEQ ID NO:77 from nucleotide 170 to nucleotide 1435, and extending contiguously from a nucleotide sequence corresponding to the 5′ end of said sequence of SEQ ID NO:77 from nucleotide 170 to nucleotide 1435, to a nucleotide sequence corresponding to the 3′ end of said sequence of SEQ ID NO:77 from nucleotide 170 to nucleotide 1435.

In other embodiments, the present invention provides a composition comprising a protein, wherein said protein comprises an amino acid sequence selected from the group consisting of:

(a) the amino acid sequence of SEQ ID NO:78;

(b) fragments of the amino acid sequence of SEQ ID NO:78, each fragment comprising eight consecutive amino acids of SEQ ID NO:78; and

(c) the amino acid sequence encoded by the cDNA insert of clone nn16_{—}10 deposited with the ATCC under accession number 98629;

the protein being substantially free from other mammalian proteins. Preferably such protein comprises the amino acid sequence of SEQ ID NO:78. In further preferred embodiments, the present invention provides a protein comprising a fragment of the amino acid sequence of SEQ ID NO:78 having biological activity, the fragment preferably comprising eight (more preferably twenty, most preferably thirty) consecutive amino acids of SEQ ID NO:78, or a protein comprising a fragment of the amino acid sequence of SEQ ID NO:78 having biological activity, the fragment comprising the amino acid sequence from amino acid 218 to amino acid 227 of SEQ ID NO:78.

In one embodiment, the present invention provides a composition comprising an isolated polynucleotide selected from the group consisting of:

(a) a polynucleotide comprising the nucleotide sequence of SEQ ID NO:79;

(b) a polynucleotide comprising the nucleotide sequence of SEQ ID NO:79 from nucleotide 1567 to nucleotide 1809;

(c) a polynucleotide comprising the nucleotide sequence of SEQ ID NO:79 from nucleotide 1726 to nucleotide 1809;

(d) a polynucleotide comprising the nucleotide sequence of the full-length protein coding sequence of clone np189_{—}9 deposited with the ATCC under accession number 98629;

(e) a polynucleotide encoding the full-length protein encoded by the cDNA insert of clone np189_{—}9 deposited with the ATCC under accession number 98629;

(f) a polynucleotide comprising the nucleotide sequence of a mature protein coding sequence of clone np189_{—}9 deposited with the ATCC under accession number 98629;

(g) a polynucleotide encoding a mature protein encoded by the cDNA insert of clone np189_{—}9 deposited with the ATCC under accession number 98629;

(h) a polynucleotide encoding a protein comprising the amino acid sequence of SEQ ID NO:80;

(i) a polynucleotide encoding a protein comprising a fragment of the amino acid sequence of SEQ ID NO:80 having biological activity, the fragment comprising eight consecutive amino acids of SEQ ID NO:80;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:79 from nucleotide 1567 to nucleotide 1809; the nucleotide sequence of SEQ ID NO:79 from nucleotide 1726 to nucleotide 1809; the nucleotide sequence of the full-length protein coding sequence of clone np189_{—}9 deposited with the ATCC under accession number 98629; or the nucleotide sequence of a mature protein coding sequence of clone np189_{—}9 deposited with the ATCC under accession number 98629. In other preferred embodiments, the polynucleotide encodes the full-length or a mature protein encoded by the cDNA insert of clone np189_{—}9 deposited with the ATCC under accession number 98629. In further preferred embodiments, the present invention provides a polynucleotide encoding a protein comprising a fragment of the amino acid sequence of SEQ ID NO:80 having biological activity, the fragment preferably comprising eight (more preferably twenty, most preferably thirty) consecutive amino acids of SEQ ID NO: 80, or a polynucleotide encoding a protein comprising a fragment of the amino acid sequence of SEQ ID NO:80 having biological activity, the fragment comprising the amino acid sequence from amino acid 35 to amino acid 44 of SEQ ID NO:80.

Other embodiments provide the gene corresponding to the cDNA sequence of SEQ ID NO:79.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:79, but excluding the poly(A) tail at the 3′ end of SEQ ID NO:79; and

(ab) the nucleotide sequence of the cDNA insert of clone np189_{—}9 deposited with the ATCC under accession number 98629;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:79, but excluding the poly(A) tail at the 3′ end of SEQ ID NO:79; and

(bb) the nucleotide sequence of the cDNA insert of clone np189_{—}9 deposited with the ATCC under accession number 98629;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:79, and extending contiguously from a nucleotide sequence corresponding to the 5′ end of SEQ ID NO:79 to a nucleotide sequence corresponding to the 3′ end of SEQ ID NO:79, but excluding the poly(A) tail at the 3′ end of SEQ ID NO:79. Also preferably the polynucleotide isolated according to the above process comprises a nucleotide sequence corresponding to the cDNA sequence of SEQ ID NO:79 from nucleotide 1567 to nucleotide 1809, and extending contiguously from a nucleotide sequence corresponding to the 5′ end of said sequence of SEQ ID NO:79 from nucleotide 1567 to nucleotide 1809, to a nucleotide sequence corresponding to the 3′ end of said sequence of SEQ ID NO:79 from nucleotide 1567 to nucleotide 1809. Also preferably the polynucleotide isolated according to the above process comprises a nucleotide sequence corresponding to the cDNA sequence of SEQ ID NO:79 from nucleotide 1726 to nucleotide 1809, and extending contiguously from a nucleotide sequence corresponding to the 5′ end of said sequence of SEQ ID NO:79 from nucleotide 1726 to nucleotide 1809, to a nucleotide sequence corresponding to the 3′ end of said sequence of SEQ ID NO:79 from nucleotide 1726 to nucleotide 1809.

In other embodiments, the present invention provides a composition comprising a protein, wherein said protein comprises an amino acid sequence selected from the group consisting of:

(a) the amino acid sequence of SEQ ID NO:80;

(b) fragments of the amino acid sequence of SEQ ID NO:80, each fragment comprising eight consecutive amino acids of SEQ ID NO:80; and

(c) the amino acid sequence encoded by the cDNA insert of clone np189_{—}9 deposited with the ATCC under accession number 98629;

the protein being substantially free from other marunalian proteins. Preferably such protein comprises the amino acid sequence of SEQ ID NO:80. In further preferred embodiments, the present invention provides a protein comprising a fragment of the amino acid sequence of SEQ ID NO:80 having biological activity, the fragment preferably comprising eight (more preferably twenty, most preferably thirty) consecutive amino acids of SEQ ID NO:80, or a protein comprising a fragment of the amino acid sequence of SEQ ID NO:80 having biological activity, the fragment comprising the amino acid sequence from amino acid 35 to amino acid 44 of SEQ ID NO:80.

In one embodiment, the present invention provides a composition comprising an isolated polynucleotide selected from the group consisting of:

(a) a polynucleotide comprising the nucleotide sequence of SEQ ID NO:81;

(b) a polynucleotide comprising the nucleotide sequence of SEQ ID NO:81 from nucleotide 2054 to nucleotide 2206;

(c) a polynucleotide comprising the nucleotide sequence of the full-length protein coding sequence of clone ny226_{—}1 deposited with the ATCC under accession number 98629;

(d) a polynucleotide encoding the full-length protein encoded by the cDNA insert of clone ny226_{—}1 deposited with the ATCC under accession number 98629;

(e) a polynucleotide comprising the nucleotide sequence of a mature protein coding sequence of clone ny226_{—}1 deposited with the ATCC under accession number 98629;

(f) a polynucleotide encoding a mature protein encoded by the cDNA insert of clone ny226_{—}1 deposited with the ATCC under accession number 98629;

(g) a polynucleotide encoding a protein comprising the amino acid sequence of SEQ ID NO:82;

(h) a polynucleotide encoding a protein comprising a fragment of the amino acid sequence of SEQ ID NO:82 having biological activity, the fragment comprising eight consecutive amino acids of SEQ ID NO:82;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:81 from nucleotide 2054 to nucleotide 2206; the nucleotide sequence of the full-length protein coding sequence of clone ny226_{—}1 deposited with the ATCC under accession number 98629; or the nucleotide sequence of a mature protein coding sequence of clone ny226_{—}1 deposited with the ATCC under accession number 98629. In other preferred embodiments, the polynucleotide encodes the full-length or a mature protein encoded by the cDNA insert of clone ny226_{—}1 deposited with the ATCC under accession number 98629. In further preferred embodiments, the present invention provides a polynucleotide encoding a protein comprising a fragment of the amino acid sequence of SEQ ID NO:82 having biological activity, the fragment preferably comprising eight (more preferably twenty, most preferably thirty) consecutive amino acids of SEQ ID NO:82, or a polynucleotide encoding a protein comprising a fragment of the amino acid sequence of SEQ ID NO:82 having biological activity, the fragment comprising the amino acid sequence from amino acid 20 to amino acid 29 of SEQ ID NO:82.

Other embodiments provide the gene corresponding to the cDNA sequence of SEQ ID NO:81.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:81, but excluding the poly(A) tail at the 3′ end of SEQ ID NO:81; and

(ab) the nucleotide sequence of the cDNA insert of clone ny226_{—}1 deposited with the ATCC under accession number 98629;

(ii) hybridizing said probe(s) to human genomnic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:81, but excluding the poly(A) tail at the 3′ end of SEQ ID NO:81; and

(bb) the nucleotide sequence of the cDNA insert of clone ny226_{—}1 deposited with the ATCC under accession number 98629;

(ii) hybridizing said primer(s) to human genornic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:81, and extending contiguously from a nucleotide sequence corresponding to the 5′ end of SEQ ID NO:81 to a nucleotide sequence corresponding to the 3′ end of SEQ ID NO:81, but excluding the poly(A) tail at the 3′ end of SEQ ID NO:81. Also preferably the polynucleotide isolated according to the above process comprises a nucleotide sequence corresponding to the cDNA sequence of SEQ ID NO:81 from nucleotide 2054 to nucleotide 2206, and extending contiguously from a nucleotide sequence corresponding to the 5′ end of said sequence of SEQ ID NO:81 from nucleotide 2054 to nucleotide 2206, to a nucleotide sequence corresponding to the 3′ end of said sequence of SEQ ID NO:81 from nucleotide 2054 to nucleotide 2206.

In other embodiments, the present invention provides a composition comprising a protein, wherein said protein comprises an amino acid sequence selected from the group consisting of:

(a) the amino acid sequence of SEQ ID NO:82;

(b) fragments of the amino acid sequence of SEQ ID NO:82, each fragment comprising eight consecutive amino acids of SEQ ID NO:82; and

(c) the amino acid sequence encoded by the cDNA insert of clone ny226_{—}1 deposited with the ATCC under accession number 98629;

the protein being substantially free from other mammalian proteins. Preferably such protein comprises the amino acid sequence of SEQ ID NO:82. In further preferred embodiments, the present invention provides a protein comprising a fragment of the amino acid sequence of SEQ ID NO:82 having biological activity, the fragment preferably comprising eight (more preferably twenty, most preferably thirty) consecutive amino acids of SEQ ID NO:82, or a protein comprising a fragment of the amino acid sequence of SEQ ID NO:82 having biological activity, the fragment comprising the amino acid sequence from amino acid 20 to amino acid 29 of SEQ ID NO:82.

In one embodiment, the present invention provides a composition comprising an isolated polynucleotide selected from the group consisting of:

(a) a polynucleotide comprising the nucleotide sequence of SEQ ID NO:83;

(b) a polynucleotide comprising the nucleotide sequence of SEQ ID NO:83 from nucleotide 567 to nucleotide 701;

(c) a polynucleotide comprising the nucleotide sequence of the full-length protein coding sequence of clone pe159_{—}1 deposited with the ATCC under accession number 98629;

(d) a polynucleotide encoding the full-length protein encoded by the cDNA insert of clone pe159_{—}1 deposited with the ATCC under accession number 98629;

(e) a polynucleotide comprising the nucleotide sequence of a mature protein coding sequence of clone pe159_{—}1 deposited with the ATCC under accession number 98629;

(e) a polynucleotide encoding a mature protein encoded by the cDNA insert of clone pe159_{—}1 deposited with the ATCC under accession number 98629;

(g) a polynucleotide encoding a protein comprising the amino acid sequence of SEQ ID NO:84;

(h) a polynucleotide encoding a protein comprising a fragment of the amino acid sequence of SEQ ID NO:84 having biological activity, the fragment comprising eight consecutive amino acids of SEQ ID NO:84;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:83 from nucleotide 567 to nucleotide 701; the nucleotide sequence of the full-length protein coding sequence of clone pe159_{—}1 deposited with the ATCC under accession number 98629; or the nucleotide sequence of a mature protein coding sequence of clone pe159_{—}1 deposited with the ATCC under accession number 98629. In other preferred embodiments, the polynucleotide encodes the full-length or a mature protein encoded by the cDNA insert of clone pe159_{—}1 deposited with the ATCC under accession number 98629. In further preferred embodiments, the present invention provides a polynucleotide encoding a protein comprising a fragment of the amino acid sequence of SEQ ID NO:84 having biological activity, the fragment preferably comprising eight (more preferably twenty, most preferably thirty) consecutive amino acids of SEQ ID NO:84, or a polynucleotide encoding a protein comprising a fragment of the amino acid sequence of SEQ ID NO:84 having biological activity, the fragment comprising the amino acid sequence from amino acid 17 to amino acid 26 of SEQ ID NO:84.

Other embodiments provide the gene corresponding to the cDNA sequence of SEQ ID NO:83.

Further embodiments of the invention provide isolated polynucleotides produced according to a process selected from the group consisting of:

(a) a process comprising the steps of:

(i) preparing one ormorepolynucleotide probes thathybridize in 6×SSC at 65 degrees C to a nucleotide sequence selected from the group consisting of:

(aa) SEQ ID NO:83, but excluding the poly(A) tail at the 3′ end of SEQ ID NO:83; and

(ab) the nucleotide sequence of the cDNA insert of clone pe159_{—}1 deposited with the ATCC under accession number 98629;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:83, but excluding the poly(A) tail at the 3′ end of SEQ ID NO:83; and

(bb) the nucleotide sequence of the cDNA insert of clone pe159_{—}1 deposited with the ATCC under accession number 98629;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:83, and extending contiguously from a nucleotide sequence corresponding to the 5′ end of SEQ ID NO:83 to a nucleotide sequence corresponding to the 3′ end of SEQ ID NO:83, but excluding the poly(A) tail at the 3′ end of SEQ ID NO:83. Also preferably the polynucleotide isolated according to the above process comprises a nucleotide sequence corresponding to the cDNA sequence of SEQ ID NO:83 from nucleotide 567 to nucleotide 701, and extending contiguously from a nucleotide sequence corresponding to the 5′ end of said sequence of SEQ ID NO:83 from nucleotide 567 to nucleotide 701, to a nucleotide sequence corresponding to the 3′ end of said sequence of SEQ ID NO:83 from nucleotide 567 to nucleotide 701.

In other embodiments, the present invention provides a composition comprising a protein, wherein said protein comprises an amino acid sequence selected from the group consisting of:

(a) the amino acid sequence of SEQ ID NO:84;

(b) fragments of the amino acid sequence of SEQ ID NO:84, each fragment comprising eight consecutive amino acids of SEQ ID NO:84; and

(c) the amino acid sequence encoded by the cDNA insert of clone pe159_{—}1 deposited with the ATCC under accession number 98629;

the protein being substantially free from other mammalian proteins. Preferably such protein comprises the amino acid sequence of SEQ ID NO:84. In further preferred embodiments, the present invention provides a protein comprising a fragment of the amino acid sequence of SEQ ID NO:84 having biological activity, the fragment preferably comprising eight (more preferably twenty, most preferably thirty) consecutive amino acids of SEQ ID NO:84, or a protein comprising a fragment of the amino acid sequence of SEQ ID NO:84 having biological activity, the fragment comprising the amino acid sequence from amino acid 17 to amino acid 26 of SEQ ID NO:84.

In one embodiment, the present invention provides a composition comprising an isolated polynucleotide selected from the group consisting of:

(a) a polynucleotide comprising the nucleotide sequence of SEQ ID NO:85;

(b) a polynucleotide comprising the nucleotide sequence of SEQ ID NO:85 from nucleotide 593 to nucleotide 784;

(c) a polynucleotide comprising the nucleotide sequence of SEQ ID NO:85 from nucleotide 698 to nucleotide 784;

(d) a polynucleotide comprising the nucleotide sequence of the full-length protein coding sequence of clone pj314_{—}8 deposited with the ATCC under accession number 98629;

(e) a polynucleotide encoding the full-length protein encoded by the cDNA insert of clone pj314_{—}8 deposited with the ATCC under accession number 98629;

(f) a polynucleotide comprising the nucleotide sequence of a mature protein coding sequence of clone pj314_{—}8 deposited with the ATCC under accession number 98629;

(g) a polynucleotide encoding a mature protein encoded by the cDNA insert of clone pj314_{—}8 deposited with the ATCC under accession number 98629;

(h) a polynucleotide encoding a protein comprising the amino acid sequence of SEQ ID NO:86;

(i) a polynucleotide encoding a protein comprising a fragment of the amino acid sequence of SEQ ID NO:86 having biological activity, the fragment comprising eight consecutive amino acids of SEQ ID NO:86;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:85 from nucleotide 593 to nucleotide 784; the nucleotide sequence of SEQ ID NO:85 from nucleotide 698 to nucleotide 784; the nucleotide sequence of the full-length protein coding sequence of clone pj314_{—}8 deposited with the ATCC under accession number 98629; or the nucleotide sequence of a mature protein coding sequence of clone pj314_{—}8 deposited with the ATCC under accession number 98629. In other preferred embodiments, the polynucleotide encodes the full-length or a mature protein encoded by the cDNA insert of clone pj314_{—}8 deposited with the ATCC under accession number 98629. In further preferred embodiments, the present invention provides a polynucleotide encoding a protein comprising a fragment of the amino acid sequence of SEQ ID NO:86 having biological activity, the fragment preferably comprising eight (more preferably twenty, most preferably thirty) consecutive amino acids of SEQ ID NO:86, or a polynucleotide encoding a protein comprising a fragment of the amino acid sequence of SEQ ID NO:86 having biological activity, the fragment comprising the amino acid sequence from amino acid 27 to amino acid 36 of SEQ ID NO:86.

Other embodiments provide the gene corresponding to the cDNA sequence of SEQ ID NO:85.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:85, but excluding the poly(A) tail at the 3′ end of SEQ ID NO:85; and

(ab) the nucleotide sequence of the cDNA insert of clone pj314_{—}8 deposited with the ATCC under accession number 98629;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:85, but excluding the poly(A) tail at the 3′ end of SEQ ID NO:85; and

(bb) the nucleotide sequence of the cDNA insert of clone pj314_{—}8 deposited with the ATCC under accession number 98629;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:85, and extending contiguously from a nucleotide sequence corresponding to the 5′ end of SEQ ID NO:85 to a nucleotide sequence corresponding to the 3′ end of SEQ ID NO:85, but excluding the poly(A) tail at the 3′ end of SEQ ID NO:85. Also preferably the polynucleotide isolated according to the above process comprises a nucleotide sequence corresponding to the cDNA sequence of SEQ ID NO:85 fromnucleotide 593 tonucleotide 784, and extending contiguously from a nucleotide sequence corresponding to the 5′ end of said sequence of SEQ ID NO:85 from nucleotide 593 to nucleotide 784, to a nucleotide sequence corresponding to the 3′ end of said sequence of SEQ ID NO:85 from nucleotide 593 to nucleotide 784. Also preferably the polynucleotide isolated according to the above process comprises a nucleotide sequence corresponding to the cDNA sequence of SEQ ID NO:85 from nucleotide 698 to nucleotide 784, and extending contiguously from a nucleotide sequence corresponding to the 5′ end of said sequence of SEQ ID NO:85 from nucleotide 698 to nucleotide 784, to a nucleotide sequence corresponding to the 3′ end of said sequence of SEQ ID NO:85 from nucleotide 698 to nucleotide 784.

In other embodiments, the present invention provides a composition comprising a protein, wherein said protein comprises an amino acid sequence selected from the group consisting of:

(a) the amino acid sequence of SEQ ID NO:86;

(b) fragments of the amino acid sequence of SEQ ID NO:86, each fragment comprising eight consecutive amino acids of SEQ ID NO:86; and

(c) the amino acid sequence encoded by the cDNA insert of clone pj314_{—}8 deposited with the ATCC under accession number 98629;

the protein being substantially free from other mammalian proteins. Preferably such protein comprises the amino acid sequence of SEQ ID NO:86. In further preferred embodiments, the present invention provides a protein comprising a fragment of the amino acid sequence of SEQ ID NO:86 having biological activity, the fragment preferably comprising eight (more preferably twenty, most preferably thirty) consecutive amino acids of SEQ ID NO:86, or a protein comprising a fragment of the amino acid sequence of SEQ ID NO:86 having biological activity, the fragment comprising the amino acid sequence from amino acid 27 to amino acid 36 of SEQ ID NO:86.

In one embodiment, the present invention provides a composition comprising an isolated polynucleotide selected from the group consisting of:

(a) a polynucleotide comprising the nucleotide sequence of SEQ ID NO:87;

(b) a polynucleotide comprising the nucleotide sequence of SEQ ID NO:87 from nucleotide 176 to nucleotide 328;

(c) a polynucleotide comprising the nucleotide sequence of SEQ ID NO:87 from nucleotide 239 to nucleotide 328;

(d) a polynucleotide comprising the nucleotide sequence of SEQ ID NO:87 from nucleotide 1 to nucleotide 512;

(e) a polynucleotide comprising the nucleotide sequence of the full-length protein coding sequence of clone bp870_{—}1 deposited with the ATCC under accession number 98724;

(f) a polynucleotide encoding the full-length protein encoded by the cDNA insert of clone bp870_{—}1 deposited with the ATCC under accession number 98724;

(g) a polynucleotide comprising the nucleotide sequence of a mature protein coding sequence of clone bp870_{—}1 deposited with the ATCC under accession number 98724;

(h) a polynucleotide encoding a mature protein encoded by the cDNA insert of clone bp870_{—}1 deposited with the ATCC under accession number 98724;

(i) a polynucleotide encoding a protein comprising the amino acid sequence of SEQ ID NO:88;

(j) a polynucleotide encoding a protein comprising a fragment of the amino acid sequence of SEQ ID NO:88 having biological activity, the fragment comprising eight consecutive amino acids of SEQ ID NO:88;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:87 from nucleotide 176 to nucleotide 328; the nucleotide sequence of SEQ ID NO:87 from nucleotide 239 to nucleotide 328; the nucleotide sequence of SEQ ID NO:87 from nucleotide 1 to nucleotide 512; the nucleotide sequence of the full-length protein coding sequence of clone bp870_{—}1 deposited with the ATCC under accession number 98724; or the nucleotide sequence of a mature protein coding sequence of clone bp870_{—}1 deposited with the ATCC under accession number 98724. In other preferred embodiments, the polynucleotide encodes the full-length or a mature protein encoded by the cDNA insert of clone bp870_{—}1 deposited with the ATCC under accession number 98724. In further preferred embodiments, the present invention provides a polynucleotide encoding a protein comprising a fragment of the amino acid sequence of SEQ ID NO:88 having biological activity, the fragment preferably comprising eight (more preferably twenty, most preferably thirty) consecutive amino acids of SEQ ID NO:88, or a polynucleotide encoding a protein comprising a fragment of the amino acid sequence of SEQ ID NO:88 having biological activity, the fragment comprising the amino acid sequence from amino acid 20 to amino acid 29 of SEQ ID NO:88.

Other embodiments provide the gene corresponding to the cDNA sequence of SEQ ID NO:87.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:87, but excluding the poly(A) tail at the 3′ end of SEQ ID NO:87; and

(ab) the nucleotide sequence of the cDNA insert of clone bp870_{—}1 deposited with the ATCC under accession number 98724;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:87, but excluding the poly(A) tail at the 3′ end of SEQ ID NO:87; and

(bb) the nucleotide sequence of the cDNA insert of clone bp870_{—}1 deposited with the ATCC under accession number 98724;

(ii) hybridizing said primer(s) to human genormic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:87, and extending contiguously from a nucleotide sequence corresponding to the 5′ end of SEQ ID NO:87 to a nucleotide sequence corresponding to the 3′ end of SEQ ID NO:87, but excluding the poly(A) tail at the 3′ end of SEQ ID NO:87. Also preferably the polynucleotide isolated according to the above process comprises a nucleotide sequence corresponding to the cDNA sequence of SEQ ID NO:87 from nucleotide 176 to nucleotide 328, and extending contiguously from a nucleotide sequence corresponding to the 5′ end of said sequence of SEQ ID NO:87 from nucleotide 176 to nucleotide 328, to a nucleotide sequence corresponding to the 3′ end of said sequence of SEQ ID NO:87 from nucleotide 176 to nucleotide 328. Also preferably the polynucleotide isolated according to the above process comprises a nucleotide sequence corresponding to the cDNA sequence of SEQ ID NO:87 from nucleotide 239 to nucleotide 328, and extending contiguously from a nucleotide sequence corresponding to the 5′ end of said sequence of SEQ ID NO:87 from nucleotide 239 to nucleotide 328, to a nucleotide sequence corresponding to the 3′ end of said sequence of SEQ ID NO:87 from nucleotide 239 to nucleotide 328. Also preferably the polynucleotide isolated according to the above process comprises a nucleotide sequence corresponding to the cDNA sequence of SEQ ID NO:87 from nucleotide 1 to nucleotide 512, and extending contiguously from a nucleotide sequence corresponding to the 5′ end of said sequence of SEQ ID NO:87 from nucleotide 1 to nucleotide 512, to a nucleotide sequence corresponding to the 3′ end of said sequence of SEQ ID NO:87 from nucleotide 1 to nucleotide 512.

In other embodiments, the present invention provides a composition comprising a protein, wherein said protein comprises an amino acid sequence selected from the group consisting of:

(a) the amino acid sequence of SEQ ID NO:88;

(b) fragments of the amino acid sequence of SEQ ID NO:88, each fragment comprising eight consecutive amino acids of SEQ ID NO:88; and

(c) the amino acid sequence encoded by the cDNA insert of clone bp870_{—}1 deposited with the ATCC under accession number 98724;

the protein being substantially free from other mammalian proteins. Preferably such protein comprises the amino acid sequence of SEQ ID NO:88. In further preferred embodiments, the present invention provides a protein comprising a fragment of the amino acid sequence of SEQ ID NO:88 having biological activity, the fragment preferably comprising eight (more preferably twenty, most preferably thirty) consecutive amino acids of SEQ ID NO:88, or a protein comprising a fragment of the amino acid sequence of SEQ ID NO:88 having biological activity, the fragment comprising the amino acid sequence from amino acid 20 to amino acid 29 of SEQ ID NO:88.

In one embodiment, the present invention provides a composition comprising an isolated polynucleotide selected from the group consisting of:

(a) a polynucleotide comprising the nucleotide sequence of SEQ ID NO:89;

(b) a polynucleotide comprising the nucleotide sequence of SEQ ID NO:89 from nucleotide 15 to nucleotide 749;

(c) a polynucleotide comprising the nucleotide sequence of SEQ ID NO:89 from nucleotide 141 to nucleotide 749;

(d) a polynucleotide comprising the nucleotide sequence of the full-length protein coding sequence of clone bx141_{—}2 deposited with the ATCC under accession number 98630;

(e) a polynucleotide encoding the full-length protein encoded by the cDNA insert of clone bx141_{—}2 deposited with the ATCC under accession number 98630;

(f) a polynucleotide comprising the nucleotide sequence of a mature protein coding sequence of clone bx141_{—}2 deposited with the ATCC under accession number 98630;

(g) a polynucleotide encoding a mature protein encoded by the cDNA insert of clone bx141_{—}2 deposited with the ATCC under accession number 98630;

(h) a polynucleotide encoding a protein comprising the amino acid sequence of SEQ ID NO:90;

(i) a polynucleotide encoding a protein comprising a fragment of the amnino acid sequence of SEQ ID NO:90 having biological activity, the fragment comprising eight consecutive amino acids of SEQ ID NO:90;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:89 from nucleotide 15 to nucleotide 749; the nucleotide sequence of SEQ ID NO:89 from nucleotide 141 to nucleotide 749; the nucleotide sequence of the full-length protein coding sequence of clone bx141_{—}2 deposited with the ATCC under accession number 98630; or the nucleotide sequence of a mature protein coding sequence of clone bx141_{—}2 deposited with the ATCC under accession number 98630. In other preferred embodiments, the polynucleotide encodes the full-length or a mature protein encoded by the cDNA insert of clone bx141_{—}2 deposited with the ATCC under accession number 98630. In yet other preferred embodiments, the present invention provides a polynucleotide encoding a protein comprising the amino acid sequence of SEQ ID NO:90 from amino acid 1 to amino acid 122. In further preferred embodiments, the present invention provides a polynucleotide encoding a protein comprising a fragment of the amino acid sequence of SEQ ID NO:90 having biological activity, the fragment preferably comprising eight (more preferably twenty, most preferably thirty) consecutive amino acids of SEQ ID NO:90, or a polynucleotide encoding a protein comprising a fragment of the amino acid sequence of SEQ ID NO:90 having biological activity, the fragment comprising the amino acid sequence from amino acid 117 to amino acid 126 of SEQ ID NO:90.

Other embodiments provide the gene corresponding to the cDNA sequence of SEQ ID NO:89.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:89, but excluding the poly(A) tail at the 3′ end of SEQ ID NO:89; and

(ab) the nucleotide sequence of the cDNA insert of clone bx141_{—}2 deposited with the ATCC under accession number 98630;

(ii) hybridizing said probe(s) to human genomiic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:89, but excluding the poly(A) tail at the 3′ end of SEQ ID NO:89; and

(bb) the nucleotide sequence of the cDNA insert of clone bx141_{—}2 deposited with the ATCC under accession number 98630;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:89, and extending contiguously from a nucleotide sequence corresponding to the 5′ end of SEQ ID NO:89 to a nucleotide sequence corresponding to the 3′ end of SEQ ID NO:89, but excluding the poly(A) tail at the 3′ end of SEQ ID NO:89. Also preferably the polynucleotide isolated according to the above process comprises a nucleotide sequence corresponding to the cDNA sequence of SEQ ID NO:89 from nucleotide 15 to nucleotide 749, and extending contiguously from a nucleotide sequence corresponding to the 5′ end of said sequence of SEQ ID NO:89 from nucleotide 15 to nucleotide 749, to a nucleotide sequence corresponding to the 3′ end of said sequence of SEQ ID NO:89 from nucleotide to nucleotide 749. Also preferably the polynucleotide isolated according to the above process comprises a nucleotide sequence corresponding to the cDNA sequence of SEQ ID NO:89 from nucleotide 141 to nucleotide 749, and extending contiguously from a nucleotide sequence corresponding to the 5′ end of said sequence of SEQ ID NO:89 from nucleotide 141 to nucleotide 749, to a nucleotide sequence corresponding to the 3′ end of said sequence of SEQ ID NO:89 from nucleotide 141 to nucleotide 749.

In other embodiments, the present invention provides a composition comprising a protein, wherein said protein comprises an amino acid sequence selected from the group consisting of:

(a) the amino acid sequence of SEQ ID NO:90;

(b) the amino acid sequence of SEQ ID NO:90 from amino acid 1 to amino acid 122;

(c) fragments of the amino acid sequence of SEQ ID NO:90, each fragment comprising eight consecutive amino acids of SEQ ID NO:90; and

(d) the amino acid sequence encoded by the cDNA insert of clone bx141_{—}2 deposited with the ATCC under accession number 98630;

the protein being substantially free from other mammalian proteins. Preferably such protein comprises the amino acid sequence of SEQ ID NO:90 or the amino acid sequence of SEQ ID NO:90 from amino acid 1 to amino acid 122. In further preferred embodiments, the present invention provides a protein comprising a fragment of the amino acid sequence of SEQ ID NO:90 having biological activity, the fragment preferably comprising eight (more preferably twenty, most preferably thirty) consecutive amino acids of SEQ ID NO:90, or a protein comprising a fragment of the amino acid sequence of SEQ ID NO:90 having biological activity, the fragment comprising the amino acid sequence from amino acid 117 to amino acid 126 of SEQ ID NO:90.

In one embodiment, the present invention provides a composition comprising an isolated polynucleotide selected from the group consisting of:

(a) a polynucleotide comprising the nucleotide sequence of SEQ ID NO:91;

(b) a polynucleotide comprising the nucleotide sequence of SEQ ID NO:91 from nucleotide 100 to nucleotide 1767;

(c) a polynucleotide comprising the nucleotide sequence of SEQ ID NO:91 from nucleotide 280 to nucleotide 1767;

(d) a polynucleotide comprising the nucleotide sequence of the full-length protein coding sequence of clone cw272_{—}7 deposited with the ATCC under accession number 98630;

(e) a polynucleotide encoding the full-length protein encoded by the cDNA insert of clone cw272_{—}7 deposited with the ATCC under accession number 98630;

(f) a polynucleotide comprising the nucleotide sequence of a mature protein coding sequence of clone cw272_{—}7 deposited with the ATCC under accession number 98630;

(g) a polynucleotide encoding a mature protein encoded by the cDNA insert of clone cw272_{—}7 deposited with the ATCC under accession number 98630;

(h) a polynucleotide encoding a protein comprising the amnino acid sequence of SEQ ID NO:92;

(i) a polynucleotide encoding a protein comprising a fragment of the amino acid sequence of SEQ ID NO:92 having biological activity, the fragment comprising eight consecutive amino acids of SEQ ID NO:92;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:91 from nucleotide 100 to nucleotide 1767; the nucleotide sequence of SEQ ID NO:91 from nucleotide 280 to nucleotide 1767; the nucleotide sequence of the full-length protein coding sequence of clone cw272_{—}7 deposited with the ATCC under accession number 98630; or the nucleotide sequence of a mature protein coding sequence of clone cw272_{—}7 deposited with the ATCC under accession number 98630. In other preferred embodiments, the polynucleotide encodes the full-length or a mature protein encoded by the cDNA insert of clone cw272_{—}7 deposited with the ATCC under accession number 98630. In further preferred embodiments, the present invention provides a polynucleotide encoding a protein comprising a fragment of the amino acid sequence of SEQ ID NO:92 having biological activity, the fragment preferably comprising eight (more preferably twenty, most preferably thirty) consecutive amino acids of SEQ ID NO:92, or a polynucleotide encoding a protein comprising a fragment of the amino acid sequence of SEQ ID NO:92 having biological activity, the fragment comprising the amino acid sequence from amino acid 273 to amino acid 282 of SEQ ID NO:92.

Other embodiments provide the gene corresponding to the cDNA sequence of SEQ ID NO:91.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:91, but excluding the poly(A) tail at the 3′ end of SEQ ID NO:91; and

(ab) the nucleotide sequence of the cDNA insert of clone cw272_{—}7 deposited with the ATCC under accession number 98630;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:91, but excluding the poly(A) tail at the 3′ end of SEQ ID NO:91; and

(bb) the nucleotide sequence of the cDNA insert of clone cw272_{—}7 deposited with the ATCC under accession number 98630;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:91, and extending contiguously from a nucleotide sequence corresponding to the 5′ end of SEQ ID NO:91 to a nucleotide sequence corresponding to the 3′ end of SEQ ID NO:91, but excluding the poly(A) tail at the 3′ end of SEQ ID NO:91. Also preferably the polynucleotide isolated according to the above process comprises a nucleotide sequence corresponding to the cDNA sequence of SEQ ID NO:91 from nucleotide 100 to nucleotide 1767, and extending contiguously from a nucleotide sequence corresponding to the 5′ end of said sequence of SEQ ID NO:91 from nucleotide 100 to nucleotide 1767, to a nucleotide sequence corresponding to the 3′ end of said sequence of SEQ ID NO:91 from nucleotide 100 to nucleotide 1767. Also preferably the polynucleotide isolated according to the above process comprises a nucleotide sequence corresponding to the cDNA sequence of SEQ ID NO:91 from nucleotide 280 to nucleotide 1767, and extending contiguously from a nucleotide sequence corresponding to the 5′ end of said sequence of SEQ ID NO:91 from nucleotide 280 to nucleotide 1767, to a nucleotide sequence corresponding to the 3′ end of said sequence of SEQ ID NO:91 from nucleotide 280 to nucleotide 1767.

In other embodiments, the present invention provides a composition comprising a protein, wherein said protein comprises an amino acid sequence selected from the group consisting of:

(a) the amino acid sequence of SEQ ID NO:92;

(b) fragments of the amino acid sequence of SEQ ID NO:92, each fragment comprising eight consecutive amino acids of SEQ ID NO:92; and

(c) the amino acid sequence encoded by the cDNA insert of clone cw272_{—}7 deposited with the ATCC under accession number 98630;

the protein being substantially free from other mammalian proteins. Preferably such protein comprises the amino acid sequence of SEQ ID NO:92. In further preferred embodiments, the present invention provides a protein comprising a fragment of the amino acid sequence of SEQ ID NO:92 having biological activity, the fragment preferably comprising eight (more preferably twenty, most preferably thirty) consecutive amino acids of SEQ ID NO:92, or a protein comprising a fragment of the amino acid sequence of SEQ ID NO:92 having biological activity, the fragment comprising the amino acid sequence from amino acid 273 to amino acid 282 of SEQ ID NO:92.

In one embodiment, the present invention provides a composition comprising an isolated polynucleotide selected from the group consisting of:

(a) a polynucleotide comprising the nucleotide sequence of SEQ ID NO:93;

(b) a polynucleotide comprising the nucleotide sequence of SEQ ID NO:93 from nucleotide 49 to nucleotide 1245;

(c) a polynucleotide comprising the nucleotide sequence of SEQ ID NO:93 from nucleotide 265 to nucleotide 1245;

(d) a polynucleotide comprising the nucleotide sequence of the full-length protein coding sequence of clone nh328_{—}5 deposited with the ATCC under accession number 98630;

(e) a polynucleotide encoding the full-length protein encoded by the cDNA insert of clone nh328_{—}5 deposited with the ATCC under accession number 98630;

(f) a polynucleotide comprising the nucleotide sequence of a mature protein coding sequence of clone nh328_{—}5 deposited with the ATCC under accession number 98630;

(g) a polynucleotide encoding a mature protein encoded by the cDNA insert of clone nh328_{—}5 deposited with the ATCC under accession number 98630;

(h) a polynucleotide encoding a protein comprising the arnino acid sequence of SEQ ID NO:94;

(i) a polynucleotide encoding a protein comprising a fragment of the amino acid sequence of SEQ ID NO:94 having biological activity, the fragment comprising eight consecutive amino acids of SEQ ID NO:94;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:93 from nucleotide 49 to nucleotide 1245; the nucleotide sequence of SEQ ID NO:93 from nucleotide 265 to nucleotide 1245; the nucleotide sequence of the full-length protein coding sequence of clone nh328_{—}5 deposited with the ATCC under accession number 98630; or the nucleotide sequence of a mature protein coding sequence of clone nh328_{—}5 deposited with the ATCC under accession number 98630. In other preferred embodiments, the polynucleotide encodes the full-length or a mature protein encoded by the cDNA insert of clone nh328_{—}5 deposited with the ATCC under accession number 98630. In yet other preferred embodiments, the present invention provides a polynucleotide encoding a protein comprising the amino acid sequence of SEQ ID NO:94 from amino acid 229 to amino acid 387. in further preferred embodiments, the present invention provides a polynucleotide encoding a protein comprising a fragment of the amino acid sequence of SEQ ID NO:94 having biological activity, the fragment preferably comprising eight (more preferably twenty, most preferably thirty) consecutive amino acids of SEQ ID NO:94, or a polynucleotide encoding a protein comprising a fragment of the amino acid sequence of SEQ ID NO:94 having biological activity, the fragment comprising the amino acid sequence from amino acid 194 to amino acid 203 of SEQ ID NO:94.

Other embodiments provide the gene corresponding to the cDNA sequence of SEQ ID NO:93.

Further embodiments of the invention provide isolated polynucleotides produced according to a process selected from the group consisting of:

(a) a process comprising the steps of:

(i) preparing one ormore polynucleotide probes thathybridize in 6×SSC at 65 degrees C to a nucleotide sequence selected from the group consisting of:

(aa) SEQ ID NO:93, but excluding the poly(A) tail at the 3′ end of SEQ ID NO:93; and

(ab) the nucleotide sequence of the cDNA insert of clone nh328_{—}5 deposited with the ATCC under accession number 98630;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:93, but excluding the poly(A) tail at the 3′ end of SEQ ID NO:93; and

(bb) the nucleotide sequence of the cDNA insert of clone nh328_{—}5 deposited with the ATCC under accession number 98630;

(ii) hybridizing said primer(s) to human genomnic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:93, and extending contiguously from a nucleotide sequence corresponding to the 5′ end of SEQ ID NO:93 to a nucleotide sequence corresponding to the 3′ end of SEQ ID NO:93, but excluding the poly(A) tail at the 3′ end of SEQ ID NO:93. Also preferably the polynucleotide isolated according to the above process comprises a nucleotide sequence corresponding to the cDNA sequence of SEQ ID NO:93 from nucleotide 49 to nucleotide 1245, and extending contiguously from a nucleotide sequence corresponding to the 5′ end of said sequence of SEQ ID NO:93 from nucleotide 49 to nucleotide 1245, to a nucleotide sequence corresponding to the 3′ end of said sequence of SEQ ID NO:93 from nucleotide 49 to nucleotide 1245. Also preferably the polynucleotide isolated according to the above process comprises a nucleotide sequence corresponding to the cDNA sequence of SEQ ID NO:93 from nucleotide 265 to nucleotide 1245, and extending contiguously from a nucleotide sequence corresponding to the 5′ end of said sequence of SEQ ID NO:93 from nucleotide 265 to nucleotide 1245, to a nucleotide sequence corresponding to the 3′ end of said sequence of SEQ ID NO:93 from nucleotide 265 to nucleotide 1245.

In other embodiments, the present invention provides a composition comprising a protein, wherein said protein comprises an amino acid sequence selected from the group consisting of:

(a) the amino acid sequence of SEQ ID NO:94;

(b) the amino acid sequence of SEQ ID NO:94 from amino acid 229 to amino acid 387;

(c) fragments of the amino acid sequence of SEQ ID NO:94, each fragment comprising eight consecutive amino acids of SEQ ID NO:94; and

(d) the amino acid sequence encoded by the cDNA insert of clone nh328_{—}5 deposited with the ATCC under accession number 98630;

the protein being substantially free from other mammalian proteins. Preferably such protein comprises the amino acid sequence of SEQ ID NO:94 or the amino acid sequence of SEQ ID NO:94 from amino acid 229 to amino acid 387. In further preferred embodiments, the present invention provides a protein comprising a fragment of the amino acid sequence of SEQ ID NO:94 having biological activity, the fragment preferably comprising eight (more preferably twenty, most preferably thirty) consecutive amino acids of SEQ ID NO:94, or a protein comprising a fragment of the amino acid sequence of SEQ ID NO:94 having biological activity, the fragment comprising the amino acid sequence from amino acid 194 to amino acid 203 of SEQ ID NO:94.

In one embodiment, the present invention provides a composition comprising an isolated polynucleotide selected from the group consisting of:

(a) a polynucleotide comprising the nucleotide sequence of SEQ ID NO:95;

(b) a polynucleotide comprising the nucleotide sequence of SEQ ID NO:95 from nucleotide 166 to nucleotide 552;

(c) a polynucleotide comprising the nucleotide sequence of the full-length protein coding sequence of clone nm214_{—}3 deposited with the ATCC under accession number 98630;

(d) a polynucleotide encoding the full-length protein encoded by the cDNA insert of clone nm214_{—}3 deposited with the ATCC under accession number 98630;

(e) a polynucleotide comprising the nucleotide sequence of a mature protein coding sequence of clone nm214_{—}3 deposited with the ATCC under accession number 98630;

(f) a polynucleotide encoding a mature protein encoded by the cDNA insert of clone nm214_{—}3 deposited with the ATCC under accession number 98630;

(g) a polynucleotide encoding a protein comprising the amino acid sequence of SEQ ID NO:96;

(h) a polynucleotide encoding a protein comprising a fragment of the amino acid sequence of SEQ ID NO:96 having biological activity, the fragment comprising eight consecutive amino acids of SEQ ID NO:96;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:95 from nucleotide 166 to nucleotide 552; the nucleotide sequence of the full-length protein coding sequence of clone nm214_{—}3 deposited with the ATCC under accession number 98630; or the nucleotide sequence of a mature protein coding sequence of clone nm214_{—}3 deposited with the ATCC under accession number 98630. In other preferred embodiments, the polynucleotide encodes the full-length or a mature protein encoded by the cDNA insert of clone nm214_{—}3 deposited with the ATCC under accession number 98630. In further preferred embodiments, the present invention provides a polynucleotide encoding a protein comprising a fragment of the amino acid sequence of SEQ ID NO:96 having biological activity, the fragment preferably comprising eight (more preferably twenty, most preferably thirty) consecutive amino acids of SEQ ID NO:96, or a polynucleotide encoding a protein comprising a fragment of the amino acid sequence of SEQ ID NO:96 having biological activity, the fragment comprising the amino acid sequence from amino acid 59 to amino acid 68 of SEQ ID NO:96.

Other embodiments provide the gene corresponding to the cDNA sequence of SEQ ID NO:95.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:95, but excluding the poly(A) tail at the 3′ end of SEQ ID NO:95; and

(ab) the nucleotide sequence of the cDNA insert of clone nm214_{—}3 deposited with the ATCC under accession number 98630;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:95, but excluding the poly(A) tail at the 3′ end of SEQ ID NO:95; and

(bb) the nucleotide sequence of the cDNA insert of clone nm214_{—}3 deposited with the ATCC under accession number 98630;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:95, and extending contiguously from a nucleotide sequence corresponding to the 5′ end of SEQ ID NO:95 to a nucleotide sequence corresponding to the 3′ end of SEQ ID NO:95, but excluding the poly(A) tail at the 3′ end of SEQ ID NO:95. Also preferably the polynucleotide isolated according to the above process comprises a nucleotide sequence corresponding to the cDNA sequence of SEQ ID NO:95 from nucleotide 166 to nucleotide 552, and extending contiguously from a nucleotide sequence corresponding to the 5′ end of said sequence of SEQ ID NO:95 from nucleotide 166 to nucleotide 552, to a nucleotide sequence corresponding to the 3′ end of said sequence of SEQ ID NO:95 from nucleotide 166 to nucleotide 552.

In other embodiments, the present invention provides a composition comprising a protein, wherein said protein comprises an amino acid sequence selected from the group consisting of:

(a) the amino acid sequence of SEQ ID NO:96;

(b) fragments of the amino acid sequence of SEQ ID NO:96, each fragment comprising eight consecutive amino acids of SEQ ID NO:96; and

(c) the amino acid sequence encoded by the cDNA insert of clone nm214_{—}3 deposited with the ATCC under accession number 98630;

the protein being substantially free from other mammalian proteins. Preferably such protein comprises the amino acid sequence of SEQ ID NO:96. In further preferred embodiments, the present invention provides a protein comprising a fragment of the amino acid sequence of SEQ ID NO:96 having biological activity, the fragment preferably comprising eight (more preferably twenty, most preferably thirty) consecutive amino acids of SEQ ID NO:96, or a protein comprising a fragment of the amino acid sequence of SEQ ID NO:96 having biological activity, the fragment comprising the amino acid sequence from amino acid 59 to amino acid 68 of SEQ ID NO:96.

In one embodiment, the present invention provides a composition comprising an isolated polynucleotide selected from the group consisting of:

(a) a polynucleotide comprising the nucleotide sequence of SEQ ID NO:97;

(b) a polynucleotide comprising the nucleotide sequence of SEQ ID NO:97 from nucleotide 203 to nucleotide 1441;

(c) a polynucleotide comprising the nucleotide sequence of SEQ ID NO:97 from nucleotide 251 to nucleotide 1441;

(d) a polynucleotide comprising the nucleotide sequence of the full-length protein coding sequence of clone nn320_{—}2 deposited with the ATCC under accession number 98630;

(e) a polynucleotide encoding the full-length protein encoded by the cDNA insert of clone nn320_{—}2 deposited with the ATCC under accession number 98630;

(f) a polynucleotide comprising the nucleotide sequence of a mature protein coding sequence of clone nn320_{—}2 deposited with the ATCC under accession number 98630;

(g) a polynucleotide encoding a mature protein encoded by the cDNA insert of clone nn320_{—}2 deposited with the ATCC under accession number 98630;

(h) a polynucleotide encoding a protein comprising the amino acid sequence of SEQ ID NO:98;

(i) a polynucleotide encoding a protein comprising a fragment of the amino acid sequence of SEQ ID NO:98 having biological activity, the fragment comprising eight consecutive amino acids of SEQ ID NO:98;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:97 from nucleotide 203 to nucleotide 1441; the nucleotide sequence of SEQ ID NO:97 from nucleotide 251 to nucleotide 1441; the nucleotide sequence of the full-length protein coding sequence of clone nn320_{—}2 deposited with the ATCC under accession number 98630; or the nucleotide sequence of a mature protein coding sequence of clone nn320_{—}2 deposited with the ATCC under accession number 98630. In other preferred embodiments, the polynucleotide encodes the full-length or a mature protein encoded by the cDNA insert of clone nn320_{—}2 deposited with the ATCC under accession number 98630. In yet other preferred embodiments, the present invention provides a polynucleotide encoding a protein comprising the amino acid sequence of SEQ ID NO:98 from amino acid 1 to amino acid 92. In further preferred embodiments, the present invention provides a polynucleotide encoding a protein comprising a fragment of the amino acid sequence of SEQ ID NO:98 having biological activity, the fragment preferably comprising eight (more preferably twenty, most preferably thirty) consecutive amino acids of SEQ ID NO:98, or a polynucleotide encoding a protein comprising a fragment of the amino acid sequence of SEQ ID NO:98 having biological activity, the fragment comprising the amino acid sequence from amino acid 201 to amino acid 210 of SEQ ID NO:98.

Other embodiments provide the gene corresponding to the cDNA sequence of SEQ ID NO:97.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:97, but excluding the poly(A) tail at the 3′ end of SEQ ID NO:97; and

(ab) the nucleotide sequence of the cDNA insert of clone nn320_{—}2 deposited with the ATCC under accession number 98630;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:97, but excluding the poly(A) tail at the 3′ end of SEQ ID NO:97; and

(bb) the nucleotide sequence of the cDNA insert of clone nn320_{—}2 deposited with the ATCC under accession number 98630;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:97, and extending contiguously from a nucleotide sequence corresponding to the 5′ end of SEQ ID NO:97 to a nucleotide sequence corresponding to the 3′ end of SEQ ID NO:97, but excluding the poly(A) tail at the 3′ end of SEQ ID NO:97. Also preferably the polynucleotide isolated according to the above process comprises a nucleotide sequence corresponding to the cDNA sequence of SEQ ID NO:97 from nucleotide 203 to nucleotide 1441, and extending contiguously from a nucleotide sequence corresponding to the 5′ end of said sequence of SEQ ID NO:97 from nucleotide 203 to nucleotide 1441, to a nucleotide sequence corresponding to the 3′ end of said sequence of SEQ ID NO:97 from nucleotide 203 to nucleotide 1441. Also preferably the polynucleotide isolated according to the above process comprises a nucleotide sequence corresponding to the cDNA sequence of SEQ ID NO:97 from nucleotide 251 to nucleotide 1441, and extending contiguously from a nucleotide sequence corresponding to the 5′ end of said sequence of SEQ ID NO:97 from nucleotide 251 to nucleotide 1441, to a nucleotide sequence corresponding to the 3′ end of said sequence of SEQ ID NO:97 from nucleotide 251 to nucleotide 1441.

In other embodiments, the present invention provides a composition comprising a protein, wherein said protein comprises an amino acid sequence selected from the group consisting of:

(a) the amino acid sequence of SEQ ID NO:98;

(b) the amino acid sequence of SEQ ID NO:98 from amino acid 1 to amino acid 92;

(c) fragments of the amino acid sequence of SEQ ID NO:98, each fragment comprising eight consecutive amino acids of SEQ ID NO:98; and

(d) the amino acid sequence encoded by the cDNA insert of clone nn320_{—}2 deposited with the ATCC under accession number 98630;

the protein being substantially free from other mammalian proteins. Preferably such protein comprises the amino acid sequence of SEQ ID NO:98 or the amino acid sequence of SEQ ID NO:98 from amino acid 1 to amino acid 92. In further preferred embodiments, the present invention provides a protein comprising a fragment of the armino acid sequence of SEQ ID NO:98 having biological activity, the fragment preferably comprising eight (more preferably twenty, most preferably thirty) consecutive amino acids of SEQ ID NO:98, or a protein comprising a fragment of the amino acid sequence of SEQ ID NO:98 having biological activity, the fragment comprising the amino acid sequence from amino acid 201 to amino acid 210 of SEQ ID NO:98.

In one embodiment, the present invention provides a composition comprising an isolated polynucleotide selected from the group consisting of:

(a) a polynucleotide comprising the nucleotide sequence of SEQ ID NO:99;

(b) a polynucleotide comprising the nucleotide sequence of SEQ ID NO:99 from nucleotide 74 to nucleotide 1531;

(c) a polynucleotide comprising the nucleotide sequence of the full-length protein coding sequence of clone pp392_{—}3 deposited with the ATCC under accession number 98630;

(d) a polynucleotide encoding the full-length protein encoded by the cDNA insert of clone pp392_{—}3 deposited with the ATCC under accession number 98630;

(e) a polynucleotide comprising the nucleotide sequence of a mature protein coding sequence of clone pp392_{—}3 deposited with the ATCC under accession number 98630;

(f) a polynucleotide encoding a mature protein encoded by the cDNA insert of clone pp392_{—}3 deposited with the ATCC under accession number 98630;

(g) a polynucleotide encoding a protein comprising the amino acid sequence of SEQ ID NO:100;

(h) a polynucleotide encoding a protein comprising a fragment of the amino acid sequence of SEQ ID NO:100 having biological activity, the fragment comprising eight consecutive amino acids of SEQ ID NO:100;

(i) a polynucleotide which is an allelic variant of a polynucleotide of (a)-(fl above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:99 from nucleotide 74 to nucleotide 1531; the nucleotide sequence of the full-length protein coding sequence of clone pp392_{—}3 deposited with the ATCC under accession number 98630; or the nucleotide sequence of a mature protein coding sequence of clone pp392_{—}3 deposited with the ATCC under accession number 98630. In other preferred embodiments, the polynucleotide encodes the full-length or a mature protein encoded by the cDNA insert of clone pp392_{—}3 deposited with the ATCC under accession number 98630. In further preferred embodiments, the present invention provides a polynucleotide encoding a protein comprising a fragment of the amino acid sequence of SEQ ID NO:100 having biological activity, the fragment preferably comprising eight (more preferably twenty, most preferably thirty) consecutive amino acids of SEQ ID NO:100, or a polynucleotide encoding a protein comprising a fragment of the amino acid sequence of SEQ ID NO:100 having biological activity, the fragment comprising the amino acid sequence rrom amino acid 237 to amino acid 246 of SEQ ID NO:100.

Other embodiments provide the gene corresponding to the cDNA sequence of SEQ ID NO:99.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:99, but excluding the poly(A) tail at the 3′ end of SEQ ID NO:99; and

(ab) the nucleotide sequence of the cDNA insert of clone pp392_{—}3 deposited with the ATCC under accession number 98630;

(ii) hybridizing said probe(s) to human genomnic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:99, but excluding the poly(A) tail at the 3′ end of SEQ ID NO:99; and

(bb) the nucleotide sequence of the cDNA insert of clone pp392_{—}3 deposited with the ATCC under accession number 98630;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:99, and extending contiguously from a nucleotide sequence corresponding to the 5′ end of SEQ ID NO:99 to a nucleotide sequence corresponding to the 3′ end of SEQ ID NO:99, but excluding the poly(A) tail at the 3′ end of SEQ ID NO:99. Also preferably the polynucleotide isolated according to the above process comprises a nucleotide sequence corresponding to the cDNA sequence of SEQ ID NO:99 from nucleotide 74 to nucleotide 1531, and extending contiguously from a nucleotide sequence corresponding to the 5′ end of said sequence of SEQ ID NO:99 from nucleotide 74 to nucleotide 1531, to a nucleotide sequence corresponding to the 3′ end of said sequence of SEQ ID NO:99 from nucleotide 74 to nucleotide 1531.

In other embodiments, the present invention provides a composition comprising a protein, wherein said protein comprises an amino acid sequence selected from the group consisting of:

(a) the amino acid sequence of SEQ ID NO:100;

(b) fragments of the amino acid sequence of SEQ ID NO:100, each fragment comprising eight consecutive amino acids of SEQ ID NO:100; and

(c) the amino acid sequence encoded by the cDNA insert of clone pp392_{—}3 deposited with the ATCC under accession number 98630;

the protein being substantially free from other mammalian proteins. Preferably such protein comprises the amino acid sequence of SEQ ID NO:100. In further preferred embodiments, the present invention provides a protein comprising a fragment of the amino acid sequence of SEQ ID NO:100 having biological activity, the fragment preferably comprising eight (more preferably twenty, most preferably thirty) consecutive amino acids of SEQ ID NO:100, or a protein comprising a fragment of the amino acid sequence of SEQ ID NO:100 having biological activity, the fragment comprising the amino acid sequence from amino acid 237 to amino acid 246 of SEQ ID NO:100.

In one embodiment, the present invention provides a composition comprising an isolated polynucleotide selected from the group consisting of:

(a) a polynucleotide comprising the nucleotide sequence of SEQ ID NO:101;

(b) a polynucleotide comprising the nucleotide sequence of SEQ ID NO:101 from nucleotide 58 to nucleotide 474;

(c) a polynucleotide comprising the nucleotide sequence of SEQ ID NO:101 from nucleotide 310 to nucleotide 474;

(d) a polynucleotide comprising the nucleotide sequence of the full-length protein coding sequence of clone ya13_{—}1 deposited with the ATCC under accession number 98630;

(e) a polynucleotide encoding the full-length protein encoded by the cDNA insert of clone ya13_{—}1 deposited with the ATCC under accession number 98630;

(f) a polynucleotide comprising the nucleotide sequence of a mature protein coding sequence of clone ya13_{—}1 deposited with the ATCC under accession number 98630;

(g) a polynucleotide encoding a mature protein encoded by the cDNA insert of clone ya13_{—}1 deposited with the ATCC under accession number 98630;

(h) a polynucleotide encoding a protein comprising the amino acid sequence of SEQ ID NO:102;

(i) a polynucleotide encoding a protein comprising a fragment of the amino acid sequence of SEQ ID NO:102 having biological activity, the fragment comprising eight consecutive amino acids of SEQ ID NO:102;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:101 from nucleotide 58 to nucleotide 474; the nucleotide sequence of SEQ ID NO:101 from nucleotide 310 to nucleotide 474; the nucleotide sequence of the full-length protein coding sequence of clone ya13_{—}1 deposited with the ATCC under accession number 98630; or the nucleotide sequence of a mature protein coding sequence of clone ya13_{—}1 deposited with the ATCC under accession number 98630. In other preferred embodiments, the polynucleotide encodes the full-length or a mature protein encoded by the cDNA insert of clone ya13_{—}1 deposited with the ATCC under accession number 98630. In further preferred embodiments, the present invention provides a polynucleotide encoding a protein comprising a fragment of the amino acid sequence of SEQ ID NO:102 having biological activity, the fragment preferably comprising eight (more preferably twenty, most preferably thirty) consecutive amino acids of SEQ ID NO:102, or a polynucleotide encoding a protein comprising a fragment of the amino acid sequence of SEQ ID NO:102 having biological activity, the fragment comprising the amino acid sequence from amino acid 64 to amino acid 73 of SEQ ID NO:102.

Other embodiments provide the gene corresponding to the cDNA sequence of SEQ ID NO:101.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:101, but excluding the poly(A) tail at the 3′ end of SEQ ID NO:101; and

(ab) the nucleotide sequence of the cDNA insert of clone ya13_{—}1 deposited with the ATCC under accession number 98630;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:101, but excluding the poly(A) tail at the 3′ end of SEQ ID NO:101; and

(bb) the nucleotide sequence of the cDNA insert of clone ya13_{—}1 deposited with the ATCC under accession number 98630;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:101, and extending contiguously from a nucleotide sequence corresponding to the 5′ end of SEQ ID NO:101 to a nucleotide sequence corresponding to the 3′ end of SEQ ID NO:101, but excluding the poly(A) tail at the 3′ end of SEQ ID NO:101. Also preferably the polynucleotide isolated according to the above process comprises a nucleotide sequence corresponding to the cDNA sequence of SEQ ID NO:101 from nucleotide 58 to nucleotide 474, and extending contiguously from a nucleotide sequence corresponding to the 5′ end of said sequence of SEQ ID NO:101 from nucleotide 58 to nucleotide 474, to a nucleotide sequence corresponding to the 3′ end of said sequence of SEQ ID NO:101 from nucleotide 58 to nucleotide 474. Also preferably the polynucleotide isolated according to the above process comprises a nucleotide sequence corresponding to the cDNA sequence of SEQ ID NO:101 from nucleotide 310 to nucleotide 474, and extending contiguously from a nucleotide sequence corresponding to the 5′ end of said sequence of SEQ ID NO:101 from nucleotide 310 to nucleotide 474, to a nucleotide sequence corresponding to the 3′ end of said sequence of SEQ ID NO:101 from nucleotide 310 to nucleotide 474.

In other embodiments, the present invention provides a composition comprising a protein, wherein said protein comprises an amino acid sequence selected from the group consisting of:

(a) the amino acid sequence of SEQ ID NO:102;

(b) fragments of the amino acid sequence of SEQ ID NO:102, each fragment comprising eight consecutive amino acids of SEQ ID NO:102; and

(c) the amino acid sequence encoded by the cDNA insert of clone ya13_{—}1 deposited with the ATCC under accession number 98630;

the protein being substantially free from other marnmalian proteins. Preferably such protein comprises the amino acid sequence of SEQ ID NO:102. In further preferred embodiments, the present invention provides a protein comprising a fragment of the amino acid sequence of SEQ ID NO:102 having biological activity, the fragment preferably comprising eight (more preferably twenty, most preferably thirty) consecutive amino acids of SEQ ID NO:102, or a protein comprising a fragment of the amino acid sequence of SEQ ID NO:102 having biological activity, the fragment comprising the amino acid sequence from amino acid 64 to amino acid 73 of SEQ ID NO:102.

In one embodiment, the present invention provides a composition comprising an isolated polynucleotide selected from the group consisting of:

(a) a polynucleotide comprising the nucleotide sequence of SEQ ID NO:103;

(b) a polynucleotide comprising the nucleotide sequence of SEQ ID NO:103 from nucleotide 76 to nucleotide 540;

(c) a polynucleotide comprising the nucleotide sequence of SEQ ID NO:103 from nucleotide 196 to nucleotide 540;

(d) a polynucleotide comprising the nucleotide sequence of the full-length protein coding sequence of clone yb37_{—}1 deposited with the ATCC under accession number 98630;

(e) a polynucleotide encoding the full-length protein encoded by the cDNA insert of clone yb37_{—}1 deposited with the ATCC under accession number 98630;

(f) a polynucleotide comprising the nucleotide sequence of a mature protein coding sequence of clone yb37_{—}1 deposited with the ATCC under accession number 98630;

(g) a polynucleotide encoding a mature protein encoded by the cDNA insert of clone yb37_{—}1 deposited with the ATCC under accession number 98630;

(h) a polynucleotide encoding a protein comprising the amino acid sequence of SEQ ID NO:104;

(i) a polynucleotide encoding a protein comprising a fragment of the amino acid sequence of SEQ ID NO:104 having biological activity, the fragment comprising eight consecutive amino acids of SEQ ID NO:104;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:103 from nucleotide 76 to nucleotide 540; the nucleotide sequence of SEQ ID NO:103 from nucleotide 196 to nucleotide 540; the nucleotide sequence of the full-length protein coding sequence of clone yb37_{—}1 deposited with the ATCC under accession number 98630; or the nucleotide sequence of a mature protein coding sequence of clone yb37_{—}1 deposited with the ATCC under accession number 98630. In other preferred embodiments, the polynucleotide encodes the full-length or a mature protein encoded by the cDNA insert of clone yb37_{—}1 deposited with the ATCC under accession number 98630. In further preferred embodiments, the present invention provides a polynucleotide encoding a protein comprising a fragment of the amino acid sequence of SEQ ID NO:104 having biological activity, the fragment preferably comprising eight (more preferably twenty, most preferably thirty) consecutive amino acids of SEQ ID NO:104, or a polynucleotide encoding a protein comprising a fragment of the amino acid sequence of SEQ ID NO:104 having biological activity, the fragment comprising the amino acid sequence from amino acid 72 to amino acid 81 of SEQ ID NO:104.

Other embodiments provide the gene corresponding to the cDNA sequence of SEQ ID NO:103.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:103, but excluding the poly(A) tail at the 3′ end of SEQ ID NO:103; and

(ab) the nucleotide sequence of the cDNA insert of clone yb37_{—}1 deposited with the ATCC under accession number 98630;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:103, but excluding the poly(A) tail at the 3′ end of SEQ ID NO:103; and

(bb) the nucleotide sequence of the cDNA insert of clone yb37_{—}1 deposited with the ATCC under accession number 98630;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:103, and extending contiguously from a nucleotide sequence corresponding to the 5′ end of SEQ ID NO:103 to a nucleotide sequence corresponding to the 3′ end of SEQ ID NO:103, but excluding the poly(A) tail at the 3′ end of SEQ ID NO:103. Also preferably the polynucleotide isolated according to the above process comprises a nucleotide sequence corresponding to the cDNA sequence of SEQ ID NO:103 fromnucleotide 76 to nucleotide 540, and extending contiguously from a nucleotide sequence corresponding to the 5′ end of said sequence of SEQ ID NO:103 from nucleotide 76 to nucleotide 540, to a nucleotide sequence corresponding to the 3′ end of said sequence of SEQ ID NO:103 from nucleotide 76 to nucleotide 540. Also preferably the polynucleotide isolated according to the above process comprises a nucleotide sequence corresponding to the cDNA sequence of SEQ ID NO:103 from nucleotide 196 to nucleotide 540, and extending contiguously from a nucleotide sequence corresponding to the 5′ end of said sequence of SEQ ID NO:103 from nucleotide 196 to nucleotide 540, to a nucleotide sequence corresponding to the 3′ end of said sequence of SEQ ID NO:103 from nucleotide 196 to nucleotide 540.

In other embodiments, the present invention provides a composition comprising a protein, wherein said protein comprises an amino acid sequence selected from the group consisting of:

(a) the amino acid sequence of SEQ ID NO:104;

(b) fragments of the amino acid sequence of SEQ ID NO:104, each fragment comprising eight consecutive amino acids of SEQ ID NO:104; and

(c) the amino acid sequence encoded by the cDNA insert of clone yb37_{—}1 deposited with the ATCC under accession number 98630;

the protein being substantially free from other mammalian proteins. Preferably such protein comprises the amino acid sequence of SEQ ID NO:104. In further preferred embodiments, the present invention provides a protein comprising a fragment of the amino acid sequence of SEQ ID NO:104 having biological activity, the fragment preferably comprising eight (more preferably twenty, most preferably thirty) consecutive amino acids of SEQ ID NO:104, or a protein comprising a fragment of the amino acid sequence of SEQ ID NO:104 having biological activity, the fragment comprising the amino acid sequence from amino acid 72 to amino acid 81 of SEQ ID NO:104.

In one embodiment, the present invention provides a composition comprising an isolated polynucleotide selected from the group consisting of:

(a) a polynucleotide comprising the nucleotide sequence of SEQ ID NO:105;

(b) a polynucleotide comprising the nucleotide sequence of SEQ ID NO:105 from nucleotide 275 to nucleotide 415;

(c) a polynucleotide comprising the nucleotide sequence of SEQ ID NO:105 from nucleotide 374 to nucleotide 415;

(d) a polynucleotide comprising the nucleotide sequence of the full-length protein coding sequence of clone yb39_{—}1 deposited with the ATCC under accession number 98630;

(e) a polynucleotide encoding the full-length protein encoded by the cDNA insert of clone yb39_{—}1 deposited with the ATCC under accession number 98630;

(f) a polynucleotide comprising the nucleotide sequence of a mature protein coding sequence of clone yb39_{—}1 deposited with the ATCC under accession number 98630;

(g) a polynucleotide encoding a mature protein encoded by the cDNA insert of clone yb39_{—}1 deposited with the ATCC under accession number 98630;

(h) a polynucleotide encoding a protein comprising the amino acid sequence of SEQ ID NO:106;

(i) a polynucleotide encoding a protein comprising a fragment of the amino acid sequence of SEQ ID NO:106 having biological activity, the fragment comprising eight consecutive amino acids of SEQ ID NO:106;

(j) a polynucleotide which is an allelic variant of a polynucleotide of (a)-(g) above;

(k) a polynucleotide which encodes a species homologue of the protein of (h) or (i) above; and

(1) a polynucleotide that hybridizes under stringent conditions to any one of the polynucleotides specified in (a)-(i).

Preferably, such polynucleotide comprises the nucleotide sequence of SEQ ID NO:105 from nucleotide 275 to nucleotide 415; the nucleotide sequence of SEQ ID NO:105 from nucleotide 374 to nucleotide 415; the nucleotide sequence of the full-length protein coding sequence of clone yb39_{—}1 deposited with the ATCC under accession number 98630; or the nucleotide sequence of a mature protein coding sequence of clone yb39_{—}1 deposited with the ATCC under accession number 98630. In other preferred embodiments, the polynucleotide encodes the full-length or a mature protein encoded by the cDNA insert of clone yb39_{—}1 deposited with the ATCC under accession number 98630. In further preferred embodiments, the present invention provides a polynucleotide encoding a protein comprising a fragment of the amino acid sequence of SEQ ID NO:106 having biological activity, the fragment preferably comprising eight (more preferably twenty, most preferably thirty) consecutive amino acids of SEQ ID NO:106, or a polynucleotide encoding a protein comprising a fragment of the amino acid sequence of SEQ ID NO:106 having biological activity, the fragment comprising the amino acid sequence from amino acid 15 to amino acid 24 of SEQ ID NO:106.

Other embodiments provide the gene corresponding to the cDNA sequence of SEQ ID NO:105.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO: 105, but excluding the poly(A) tail at the 3′ end of SEQ ID NO:105; and

(ab) the nucleotide sequence of the cDNA insert of clone yb39_{—}1 deposited with the ATCC under accession number 98630;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:105,but excluding the poly(A) tail at the 3′ end of SEQ ID NO:105; and

(bb) the nucleotide sequence of the cDNA insert of clone yb39_{—}1 deposited with the ATCC under accession number 98630;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:105, and extending contiguously from a nucleotide sequence corresponding to the 5′ end of SEQ ID NO:105 to a nucleotide sequence corresponding to the 3′ end of SEQ ID NO:105 , but excluding the poly(A) tail at the 3′ end of SEQ ID NO:105. Also preferably the polynucleotide isolated according to the above process comprises a nucleotide sequence corresponding to the cDNA sequence of SEQ ID NO:105 from nucleotide 275 to nucleotide 415, and extending contiguously from a nucleotide sequence corresponding to the 5′ end of said sequence of SEQ ID NO:105 from nucleotide 275 to nucleotide 415, to a nucleotide sequence corresponding to the 3′ end of said sequence or SEQ ID NO:105 from nucleotide 275 to nucleotide 415. Also preferably the polynucleotide isolated according to the above process comprises a nucleotide sequence corresponding to the cDNA sequence of SEQ ID NO:105 from nucleotide 374 to nucleotide 415, and extending contiguously from a nucleotide sequence corresponding to the 5′ end of said sequence of SEQ ID NO:105 from nucleotide 374 to nucleotide 415, to a nucleotide sequence corresponding to the 3′ end of said sequence of SEQ ID NO:105 from nucleotide 374 to nucleotide 415.

In other embodiments, the present invention provides a composition comprising a protein, wherein said protein comprises an amino acid sequence selected from the group consisting of:

(a) the amino acid sequence of SEQ ID NO:106;

(b) fragments of the amino acid sequence of SEQ ID NO:106, each fragment comprising eight consecutive amino acids of SEQ ID NO:106; and

(c) the amino acid sequence encoded by the cDNA insert of clone yb39_{—}1 deposited with the ATCC under accession number 98630;

the protein being substantially free from other mammalian proteins. Preferably such protein comprises the amino acid sequence of SEQ ID NO:106. In further preferred embodiments, the present invention provides a protein comprising a fragment of the amino acid sequence of SEQ ID NO:106 having biological activity, the fragment preferably comprising eight (more preferably twenty, most preferably thirty) consecutive amino acids of SEQ ID NO:106, or a protein comprising a fragment of the amino acid sequence of SEQ ID NO:106 having biological activity, the fragment comprising the amino acid sequence from amino acid 15 to amino acid 24 of SEQ ID NO:106.

In one embodiment, the present invention provides a composition comprising an isolated polynucleotide selected from the group consisting of:

(a) a polynucleotide comprising the nucleotide sequence of SEQ ID NO:107;

(b) a polynucleotide comprising the nucleotide sequence of SEQ ID NO:107 from nucleotide 427 to nucleotide 1146;

(c) a polynucleotide comprising the nucleotide sequence of SEQ ID NO:107 from nucleotide 589 to nucleotide 1146;

(d) a polynucleotide comprising the nucleotide sequence of the full-length protein coding sequence of clone bd577_{—}1 deposited with the ATCC under accession number 98631;

(e) a polynucleotide encoding the full-length protein encoded by the cDNA insert of clone bd577_{—}1 deposited with the ATCC under accession number 98631;

(f) a polynucleotide comprising the nucleotide sequence of a mature protein coding sequence of clone bd577_{—}1 deposited with the ATCC under accession number 98631;

(g) a polynucleotide encoding a mature protein encoded by the cDNA insert of clone bd577_{—}1 deposited with the ATCC under accession number 98631;

(h) a polynucleotide encoding a protein comprising the arnino acid sequence of SEQ ID NO:108;

(i) a polynucleotide encoding a protein comprising a fragment of the amino acid sequence of SEQ ID NO:108 having biological activity, the fragment comprising eight consecutive an-ino acids of SEQ ID NO:108;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:107 from nucleotide 427 to nucleotide 1146; the nucleotide sequence of SEQ ID NO: 107 from nucleotide 589 to nucleotide 1146; the nucleotide sequence of the full-length protein coding sequence of clone bd577_{—}1 deposited with the ATCC under accession number 98631; or the nucleotide sequence of a mature protein coding sequence of clone bd577_{—}1 deposited with the ATCC under accession number 98631. In other preferred embodiments, the polynucleotide encodes the full-length or a mature protein encoded by the cDNA insert of clone bd577_{—}1 deposited with the ATCC under accession number 98631. In further preferred embodiments, the present invention provides a polynucleotide encoding a protein comprising a fragment of the amino acid sequence of SEQ ID NO:108 having biological activity, the fragment preferably comprising eight (more preferably twenty, most preferably thirty) consecutive amino acids of SEQ ID NO:108, or a polynucleotide encoding a protein comprising a fragment of the amino acid sequence of SEQ ID NO:108 having biological activity, the fragment comprising the amino acid sequence from amino acid 115 to amino acid 124 of SEQ ID NO:108.

Other embodiments provide the gene corresponding to the cDNA sequence of SEQ ID NO: 107.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:107, but excluding the poly(A) tail at the 3′ end of SEQ ID NO:107; and

(ab) the nucleotide sequence of the cDNA insert of clone bd577_{—}1 deposited with the ATCC under accession number 98631;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:107, but excluding the poly(A) tail at the 3′ end of SEQ ID NO:107; and

(bb) the nucleotide sequence of the cDNA insert of clone bd577_{—}1 deposited with the ATCC under accession number 98631;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:107, and extending contiguously from a nucleotide sequence corresponding to the 5′ end of SEQ ID NO:107 to a nucleotide sequence corresponding to the 3′ end of SEQ ID NO:107, but excluding the poly(A) tail at the 3′ end of SEQ ID NO:107. Also preferably the polynucleotide isolated according to the above process comprises a nucleotide sequence corresponding to the cDNA sequence of SEQ ID NO:107 from nucleotide 427 to nucleotide 1146, and extending contiguously from a nucleotide sequence corresponding to the 5′ end of said sequence of SEQ ID NO:107 from nucleotide 427 to nucleotide 1146, to a nucleotide sequence corresponding to the 3′ end of said sequence of SEQ ID NO:107 from nucleotide 427 to nucleotide 1146. Also preferably the polynucleotide isolated according to the above process comprises a nucleotide sequence corresponding to the cDNA sequence of SEQ ID NO:107 from nucleotide 589 to nucleotide 1146, and extending contiguously from a nucleotide sequence corresponding to the 5′ end of said sequence of SEQ ID NO:107 from nucleotide 589 to nucleotide 1146, to a nucleotide sequence corresponding to the 3′ end of said sequence of SEQ ID NO:107 from nucleotide 589 to nucleotide 1146.

In other embodiments, the present invention provides a composition comprising a protein, wherein said protein comprises an amino acid sequence selected from the group consisting of:

(a) the amino acid sequence of SEQ ID NO:108;

(b) fragments of the amino acid sequence of SEQ ID NO:108, each fragment comprising eight consecutive amino acids of SEQ ID NO:108; and

(c) the amino acid sequence encoded by the cDNA insert of clone bd577_{—}1 deposited with the ATCC under accession number 98631;

the protein being substantially free from other mammalian proteins. Preferably such protein comprises the amino acid sequence of SEQ ID NO:108. In further preferred embodiments, the present invention provides a protein comprising a fragment of the amino acid sequence of SEQ ID NO:108 having biological activity, the fragment preferably comprising eight (more preferably twenty, most preferably thirty) consecutive amino acids of SEQ ID NO:108, or a protein comprising a fragment of the amino acid sequence of SEQ ID NO:108 having biological activity, the fragment comprising the amino acid sequence from amino acid 115 to amino acid 124 of SEQ ID NO:108.

In one embodiment, the present invention provides a composition comprising an isolated polynucleotide selected from the group consisting of:

(a) a polynucleotide comprising the nucleotide sequence of SEQ ID NO:109;

(b) a polynucleotide comprising the nucleotide sequence of SEQ ID NO:109 from nucleotide 95 to nucleotide 1522;

(c) a polynucleotide comprising the nucleotide sequence of SEQ ID NO:109 from nucleotide 161 to nucleotide 1522;

(d) a polynucleotide comprising the nucleotide sequence of the full-length protein coding sequence of clone bv280_{—}3 deposited with the ATCC under accession number 98631;

(e) a polynucleotide encoding the full-length protein encoded by the cDNA insert of clone bv280_{—}3 deposited with the ATCC under accession number 98631;

(f) a polynucleotide comprising the nucleotide sequence of a mature protein coding sequence of clone bv280_{—}3 deposited with the ATCC under accession number 98631;

(g) a polynucleotide encoding a mature protein encoded by the cDNA insert of clone bv280_{—}3 deposited with the ATCC under accession number 98631;

(h) a polynucleotide encoding a protein comprising the amino acid sequence of SEQ ID NO:110;

(i) a polynucleotide encoding a protein comprising a fragment of the amino acid sequence of SEQ ID NO:110 having biological activity, the fragment comprising eight consecutive amino acids of SEQ ID NO:110;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:109 from nucleotide 95 to nucleotide 1522; the nucleotide sequence of SEQ ID NO: 109 from nucleotide 161 to nucleotide 1522; the nucleotide sequence of the full-length protein coding sequence of clone bv280_{—}3 deposited with the ATCC under accession number 98631; or the nucleotide sequence of a mature protein coding sequence of clone bv280_{—}3 deposited with the ATCC under accession number 98631. In other preferred embodiments, the polynucleotide encodes the full-length or a mature protein encoded by the cDNA insert of clone bv280_{—}3 deposited with the ATCC under accession number 98631. In further preferred embodiments, the present invention provides a polynucleotide encoding a protein comprising a fragment of the amino acid sequence of SEQ ID NO:110 having biological activity, the fragment preferably comprising eight (more preferably twenty, most preferably thirty) consecutive amino acids of SEQ ID NO:110, or a polynucleotide encoding a protein comprising a fragment of the amino acid sequence of SEQ ID NO:110 having biological activity, the fragment comprising the amino acid sequence from amino acid 233 to amino acid 242 of SEQ ID NO:110.

Other embodiments provide the gene corresponding to the cDNA sequence of SEQ ID NO:109.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:109, but excluding the poly(A) tail at the 3′ end of SEQ ID NO:109; and

(ab) the nucleotide sequence of the cDNA insert of clone bv280_{—}3 deposited with the ATCC under accession number 98631;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO: 109, but excluding the poly(A) tail at the 3′ end of SEQ ID NO:109; and

(bb) the nucleotide sequence of the cDNA insert of clone bv280_{—}3 deposited with the ATCC under accession number 98631;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:109, and extending contiguously from a nucleotide sequence corresponding to the 5′ end of SEQ ID NO:109 to a nucleotide sequence corresponding to the 3′ end of SEQ ID NO:109, but excluding the poly(A) tail at the 3′ end of SEQ ID NO:109. Also preferably the polynucleotide isolated according to the above process comprises a nucleotide sequence corresponding to the cDNA sequence of SEQ ID NO:109 from nucleotide 95 to nucleotide 1522, and extending contiguously from a nucleotide sequence corresponding to the 5′ end of said sequence of SEQ ID NO:109 from nucleotide 95 to nucleotide 1522, to a nucleotide sequence corresponding to the 3′ end of said sequence of SEQ ID NO:109 from nucleotide 95 to nucleotide 1522. Also preferably the polynucleotide isolated according to the above process comprises a nucleotide sequence corresponding to the cDNA sequence of SEQ ID NO:109 from nucleotide 161 to nucleotide 1522, and extending contiguously from a nucleotide sequence corresponding to the 5′ end of said sequence of SEQ ID NO:109 from nucleotide 161 to nucleotide 1522, to a nucleotide sequence corresponding to the 3′ end of said sequence of SEQ ID NO:109 from nucleotide 161 to nucleotide 1522.

In other embodiments, the present invention provides a composition comprising a protein, wherein said protein comprises an amino acid sequence selected from the group consisting of:

(a) the amino acid sequence of SEQ ID NO:110;

(b) fragments of the amino acid sequence of SEQ ID NO:110, each fragment comprising eight consecutive amino acids of SEQ ID NO:110; and

(c) the amino acid sequence encoded by the cDNA insert of clone bv280_{—}3 deposited with the ATCC under accession number 98631;

the protein being substantially free from other mammalian proteins. Preferably such protein comprises the amino acid sequence of SEQ ID NO:110. In further preferred embodiments, the present invention provides a protein comprising a fragment of the amino acid sequence of SEQ ID NO:110 having biological activity, the fragment preferably comprising eight (more preferably twenty, most preferably thirty) consecutive amino acids of SEQ ID NO:110, or a protein comprising a fragment of the amino acid sequence of SEQ ID NO:110 having biological activity, the fragment comprising the amino acid sequence from amino acid 233 to amino acid 242 of SEQ ID NO:110.

In one embodiment, the present invention provides a composition comprising an isolated polynucleotide selected from the group consisting of:

(a) a polynucleotide comprising the nucleotide sequence of SEQ ID NO:111;

(b) a polynucleotide comprising the nucleotide sequence of SEQ ID NO:111 from nucleotide 286 to nucleotide 552;

(c) a polynucleotide comprising the nucleotide sequence of SEQ ID NO:111 from nucleotide 475 to nucleotide 552;

(d) a polynucleotide comprising the nucleotide sequence of the full-length protein coding sequence of clone co315_{—}3 deposited with the ATCC under accession number 98631;

(e) a polynucleotide encoding the full-length protein encoded by the cDNA insert of clone co315_{—}3 deposited with the ATCC under accession number 98631;

(f) a polynucleotide comprising the nucleotide sequence of a mature protein coding sequence of clone co315_{—}3 deposited with the ATCC under accession number 98631;

(g) a polynucleotide encoding a mature protein encoded by the cDNA insert of clone co315_{—}3 deposited with the ATCC under accession number 98631;

(h) a polynucleotide encoding a protein comprising the amino acid sequence of SEQ ID NO:112;

(i) a polynucleotide encoding a protein comprising a fragment of the amino acid sequence of SEQ ID NO:112 having biological activity, the fragment comprising eight consecutive amino acids of SEQ ID NO:112;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:111 fromnucleotide 286 to nucleotide 552; the nucleotide sequence of SEQ ID NO:111 from nucleotide 475 to nucleotide 552; the nucleotide sequence of the full-length protein coding sequence of clone co315_{—}3 deposited with the ATCC under accession number 98631; or the nucleotide sequence of a mature protein coding sequence of clone co315_{—}3 deposited with the ATCC under accession number 98631. In other preferred embodiments, the polynucleotide encodes the full-length or a mature protein encoded by the cDNA insert of clone co315_{—}3 deposited with the ATCC under accession number 98631. In further preferred embodiments, the present invention provides a polynucleotide encoding a protein comprising a fragment of the amino acid sequence of SEQ ID NO:112 having biological activity, the fragment preferably comprising eight (more preferably twenty, most preferably thirty) consecutive amino acids of SEQ ID NO:112, or a polynucleotide encoding a protein comprising a fragment of the amino acid sequence of SEQ ID NO:112 having biological activity, the fragment comprising the amino acid sequence from amino acid 39 to amino acid 48 of SEQ ID NO:112.

Other embodiments provide the gene corresponding to the cDNA sequence of SEQ ID NO:111.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQIDNO:11,but excluding the poly(A) tail at the 3′ end of SEQ ID NO:111; and

(ab) the nucleotide sequence of the cDNA insert of clone co315_{—}3 deposited with the ATCC under accession number 98631;

(ii) hybridizing said probe(s) to human genonic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:111,but excluding the poly(A) tail at the 3′ end of SEQ ID NO:111; and

(bb) the nucleotide sequence of the cDNA insert of clone co315_{—}3 deposited with the ATCC under accession number 98631;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:111, and extending contiguously from a nucleotide sequence corresponding to the 5′ end of SEQ ID NO:111 to a nucleotide sequence corresponding to the 3′ end of SEQ ID NO:111, but excluding the poly(A) tail at the 3′ end of SEQ ID NO:111. Also preferably the polynucleotide isolated according to the above process comprises a nucleotide sequence corresponding to the cDNA sequence of SEQ ID NO:111 from nucleotide 286 to nucleotide 552, and extending contiguously from a nucleotide sequence corresponding to the 5′ end of said sequence of SEQ ID NO:111 from nucleotide 286 to nucleotide 552, to a nucleotide sequence corresponding to the 3′ end of said sequence of SEQ ID NO:111 from nucleotide 286 to nucleotide 552. Also preferably the polynucleotide isolated according to the above process comprises a nucleotide sequence corresponding to the cDNA sequence of SEQ ID NO:111 from nucleotide 475 to nucleotide 552, and extending contiguously from a nucleotide sequence corresponding to the 5′ end of said sequence of SEQ ID NO:111 from nucleotide 475 to nucleotide 552, to a nucleotide sequence corresponding to the 3′ end of said sequence of SEQ ID NO:111 from nucleotide 475 to nucleotide 552.

In other embodiments, the present invention provides a composition comprising a protein, wherein said protein comprises an amino acid sequence selected from the group consisting of:

(a) the amino acid sequence of SEQ ID NO:112;

(b) fragments of the amino acid sequence of SEQ ID NO:112, each fragment comprising eight consecutive amino acids of SEQ ID NO:112; and

(c) the amino acid sequence encoded by the cDNA insert of clone co315_{—}3 deposited with the ATCC under accession number 98631;

the protein being substantially free from other mammalian proteins. Preferably such protein comprises the amino acid sequence of SEQ ID NO:112. In further preferred embodiments, the present invention provides a protein comprising a fragment of the amino acid sequence of SEQ ID NO:112 having biological activity, the fragment preferably comprising eight (more preferably twenty, most preferably thirty) consecutive amino acids of SEQ ID NO:112, or a protein comprising a fragment of the amino acid sequence of SEQ ID NO:112 having biological activity, the fragment comprising the amino acid sequence from amino acid 39 to amino acid 48 of SEQ ID NO:112.

In one embodiment, the present invention provides a composition comprising an isolated polynucleotide selected from the group consisting of:

(a) a polynucleotide comprising the nucleotide sequence of SEQ ID NO:113;

(b) a polynudeotide comprising the nucleotide sequence of SEQ ID NO:113 from nucleotide 1682 to nucleotide 1963;

(c) a polynucleotide comprising the nucleotide sequence of the full-length protein coding sequence of clone ij226_{—}6 deposited with the ATCC under accession number 98631;

(d) a polynucleotide encoding the full-length protein encoded by the cDNA insert of clone ij226_{—}6 deposited with the ATCC under accession number 98631;

(e) a polynucleotide comprising the nucleotide sequence of a mature protein coding sequence of clone ij226_{—}6 deposited with the ATCC under accession number 98631;

(f) a polynucleotide encoding a mature protein encoded by the cDNA insert of clone ij226_{—}6 deposited with the ATCC under accession number 98631;

(g) a polynucleotide encoding a protein comprising the amino acid sequence of SEQ ID NO:114;

(h) a polynucleotide encoding a protein comprising a fragment of the amino acid sequence of SEQ ID NO:114 having biological activity, the fragment comprising eight consecutive amino acids of SEQ ID NO:114;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:113 from nucleotide 1682 to nucleotide 1963; the nucleotide sequence of the full-length protein coding sequence of clone ij226_{—}6 deposited with the ATCC under accession number 98631; or the nucleotide sequence of a mature protein coding sequence of clone ij226_{—}6 deposited with the ATCC under accession number 98631. In other preferred embodiments, the polynucleotide encodes the full-length or a mature protein encoded by the cDNA insert of clone ij226_{—}6 deposited with the ATCC under accession number 98631.

In further preferred embodiments, the present invention provides a polynucleotide encoding a protein comprising a fragment of the amino acid sequence of SEQ ID NO:114 having biological activity, the fragment preferably comprising eight (more preferably twenty, most preferably thirty) consecutive amino acids of SEQ ID NO:114, or a polynucleotide encoding a protein comprising a fragment of the amino acid sequence of SEQ ID NO:114 having biological activity, the fragment comprising the amino acid sequence from amino acid 42 to amino acid 51 of SEQ ID NO:114.

Other embodiments provide the gene corresponding to the cDNA sequence of SEQ ID NO:113.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:113, but excluding the poly(A) tail at the 3′ end of SEQ ID NO:113; and

(ab) the nucleotide sequence of the cDNA insert of clone ij226_{—}6 deposited with the ATCC under accession number 98631;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:1 13, but excluding the poly(A) tail at the 3′ end of SEQ ID NO:113; and

(bb) the nucleotide sequence of the cDNA insert of clone ij226_{—}6 deposited with the ATCC under accession number 98631;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:113, and extending contiguously from a nucleotide sequence corresponding to the 5′ end of SEQ ID NO:113 to a nucleotide sequence corresponding to the 3′ end of SEQ ID NO:113, but excluding the poly(A) tail at the 3′ end of SEQ ID NO:113. Also preferably the polynucleotide isolated according to the above process comprises a nucleotide sequence corresponding to the cDNA sequence of SEQ ID NO:113 from nucleotide 1682 to nucleotide 1963, and extending contiguously from a nucleotide sequence corresponding to the 5′ end of said sequence of SEQ ID NO:113 from nucleotide 1682 to nucleotide 1963, to a nucleotide sequence corresponding to the 3′ end of said sequence of SEQ ID NO:113 from nucleotide 1682 to nucleotide 1963.

In other embodiments, the present invention provides a composition comprising a protein, wherein said protein comprises an amino acid sequence selected from the group consisting of:

(a) the amino acid sequence of SEQ ID NO:114;

(b) fragments of the amino acid sequence of SEQ ID NO:114, each fragment comprising eight consecutive amino acids of SEQ ID NO:114; and

(c) the amino acid sequence encoded by the cDNA insert of clone ij226_{—}6 deposited with the ATCC under accession number 98631;

the protein being substantially free from other mammalian proteins. Preferably such protein comprises the amino acid sequence of SEQ ID NO:114. In further preferred embodiments, the present invention provides a protein comprising a fragment of the amino acid sequence of SEQ ID NO:114 having biological activity, the fragment preferably comprising eight (more preferably twenty, most preferably thirty) consecutive amino acids of SEQ ID NO:114, or a protein comprising a fragment of the amino acid sequence of SEQ ID NO:114 having biological activity, the fragment comprising the amino acid sequence from amino acid 42 to amino acid 51 of SEQ ID NO:114.

In one embodiment, the present invention provides a composition comprising an isolated polynucleotide selected from the group consisting of:

(a) a polynucleotide comprising the nucleotide sequence of SEQ ID NO:115;

(b) a polynucleotide comprising the nucleotide sequence of SEQ ID NO:115 from nucleotide 1137 to nucleotide 1346;

(c) a polynucleotide comprising the nucleotide sequence of the full-length protein coding sequence of clone nf443_{—}1 deposited with the ATCC under accession number 98631;

(d) a polynucleotide encoding the full-length protein encoded by the cDNA insert of clone nf443_{—}1 deposited with the ATCC under accession number 98631;

(e) a polynucleotide comprising the nucleotide sequence of a mature protein coding sequence of clone nf443_{—}1 deposited with the ATCC under accession number 98631;

(f) a polynucleotide encoding a mature protein encoded by the cDNA insert of clone nf443_{—}1 deposited with the ATCC under accession number 98631;

(g) a polynucleotide encoding a protein comprising the amino acid sequence of SEQ ID NO:116;

(h) a polynucleotide encoding a protein comprising a fragment of the amino acid sequence of SEQ ID NO:116 having biological activity, the fragment comprising eight consecutive amino acids of SEQ ID NO:116;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:115 from nucleotide 1137 to nucleotide 1346; the nucleotide sequence of the full-length protein coding sequence of clone nf443_{—}1 deposited with the ATCC under accession number 98631; or the nucleotide sequence of a mature protein coding sequence of clone nf443_{—}1 deposited with the ATCC under accession number 98631. In other preferred embodiments, the polynucleotide encodes the full-length or a mature protein encoded by the cDNA insert of clone nf443_{—}1 deposited with the ATCC under accession number 98631. In further preferred embodiments, the present invention provides a polynucleotide encoding a protein comprising a fragment of the amino acid sequence of SEQ ID NO:116 having biological activity, the fragment preferably comprising eight (more preferably twenty, most preferably thirty) consecutive amino acids of SEQ ID NO:116, or a polynucleotide encoding a protein comprising a fragment of the amino acid sequence of SEQ ID NO:116 having biological activity, the fragment comprising the amino acid sequence from amino acid 30 to amino acid 39 of SEQ ID NO:116.

Other embodiments provide the gene corresponding to the cDNA sequence of SEQ ID NO:115.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:115, but excluding the poly(A) tail at the 3′ end of SEQ ID NO:115; and

(ab) the nucleotide sequence of the cDNA insert of clone nf443_{—}1 deposited with the ATCC under accession number 98631;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:115, but excluding the poly(A) tail at the 3′ end of SEQ ID NO:115; and

(bb) the nucleotide sequence of the cDNA insert of clone nf443_{—}1 deposited with the ATCC under accession number 98631;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:115, and extending contiguously from a nucleotide sequence corresponding to the 5′ end of SEQ ID NO:115 to a nucleotide sequence corresponding to the 3′ end of SEQ ID NO:115, but excluding the poly(A) tail at the 3′ end of SEQ ID NO:115. Also preferably the polynucleotide isolated according to the above process comprises a nucleotide sequence corresponding to the cDNA sequence of SEQ ID NO:115 from nucleotide 1137 to nucleotide 1346, and extending contiguously from a nucleotide sequence corresponding to the 5′ end of said sequence of SEQ ID jNO:115 from nucleotide 1137 to nucleotide 1346, to a nucleotide sequence corresponding to the 3′ end of said sequence of SEQ ID NO:115 from nucleotide 1137 to nucleotide 1346.

In other embodiments, the present invention provides a composition comprising a protein, wherein said protein comprises an amino acid sequence selected from the group consisting of:

(a) the amino acid sequence of SEQ ID NO:116;

(b) fragments of the amino acid sequence of SEQ ID NO:116, each fragment comprising eight consecutive amino acids of SEQ ID NO:116; and

(c) the amino acid sequence encoded by the cDNA insert of clone nf443_{—}1 deposited with the ATCC under accession number 98631;

the protein being substantially free from other mammalian proteins. Preferably such protein comprises the amino acid sequence of SEQ ID NO:116. In further preferred embodiments, the present invention provides a protein comprising a fragment of the amino acid sequence of SEQ ID NO:116 having biological activity, the fragment preferably comprising eight (more preferably twenty, most preferably thirty) consecutive amino acids of SEQ ID NO:116, or a protein comprising a fragment of the amino acid sequence of SEQ ID NO:116 having biological activity, the fragment comprising the amino acid sequence from amino acid 30 to amino acid 39 of SEQ ID NO:116.

In one embodiment, the present invention provides a composition comprising an isolated polynucleotide selected from the group consisting of:

(a) a polynucleotide comprising the nucleotide sequence of SEQ ID NO:117;

(b) a polynucleotide comprising the nucleotide sequence of SEQ ID NO:117 from nucleotide 308 to nucleotide 634;

(c) a polynucleotide comprising the nucleotide sequence of the full-length protein coding sequence of clone nt429_{—}1 deposited with the ATCC under accession number 98631;

(d) a polynucleotide encoding the full-length protein encoded by the cDNA insert of clone nt429_{—}1 deposited with the ATCC under accession number 98631;

(e) a polynucleotide comprising the nucleotide sequence of a mature protein coding sequence of clone nt429_{—}1 deposited with the ATCC under accession number 98631;

(f) a polynucleotide encoding a mature protein encoded by the cDNA insert of clone nt429_{—}1 deposited with the ATCC under accession number 98631;

(g) a polynucleotide encoding a protein comprising the amino acid sequence of SEQ ID NO:118;

(h) a polynucleotide encoding a protein comprising a fragment of the amino acid sequence of SEQ ID NO:118 having biological activity, the fragment comprising eight consecutive amino acids of SEQ ID NO:118;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:117 from nucleotide 308 to nucleotide 634; the nucleotide sequence of the full-length protein coding sequence of clone nt429_{—}1 deposited with the ATCC under accession number 98631; or the nucleotide sequence of a mature protein coding sequence of clone nt429_{—}1 deposited with the ATCC under accession number 98631. In other preferred embodiments, the polynucleotide encodes the full-length or a mature protein encoded by the cDNA insert of clone nt429_{—}1 deposited with the ATCC under accession number 98631. In yet other preferred embodiments, the present invention provides a polynucleotide encoding a protein comprising the amino acid sequence of SEQ ID NO:1 18 from amino acid 1 to amino acid 47. In further preferred embodiments, the present invention provides a polynucleotide encoding a protein comprising a fragment of the amino acid sequence of SEQ ID NO:118 having biological activity, the fragment preferably comprising eight (more preferably twenty, most preferably thirty) consecutive amino acids of SEQ ID NO:118, or a polynucleotide encoding a protein comprising a fragment of the amino acid sequence of SEQ ID NO:118 having biological activity, the fragment comprising the amino acid sequence from amino acid 49 to amino acid 58 of SEQ ID NO:118.

Other embodiments provide the gene corresponding to the cDNA sequence of SEQ ID NO:117.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:117, but excluding the poly(A) tail at the 3′ end of SEQ ID NO:117; and

(ab) the nucleotide sequence of the cDNA insert of clone nt429_{—}1 deposited with the ATCC under accession number 98631;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:117, but excluding the poly(A) tail at the 3′ end of SEQ ID NO:117; and

(bb) the nucleotide sequence of the cDNA insert of clone nt429_{—}1 deposited with the ATCC under accession number 98631;

(ii) hybridizing said primer(s) to human genomic DNA in conditions at least as stringent as 4×SSC at 65 degrees C;

(iii) amplifying human DNA sequences; and

(iv) isolating the polynucleotide products of step (b) (iii).

Preferably the polynucleotide isolated according to the above process comprises a nucleotide sequence corresponding to the cDNA sequence of SEQ ID NO:117, and extending contiguously from a nucleotide sequence corresponding to the 5′ end of SEQ ID NO:117 to a nucleotide sequence corresponding to the 3′ end of SEQ ID NO:117, but excluding the poly(A) tail at the 3′ end of SEQ ID NO:117. Also preferably the polynucleotide isolated according to the above process comprises a nucleotide sequence corresponding to the cDNA sequence of SEQ ID NO:117 from nucleotide 308 to nucleotide 634, and extending contiguously from a nucleotide sequence corresponding to the 5′ end of said sequence of SEQ ID NO:117 from nucleotide 308 to nucleotide 634, to a nucleotide sequence corresponding to the 3′ end of said sequence of SEQ ID NO:117 from nucleotide 308 to nucleotide 634.

In other embodiments, the present invention provides a composition comprising a protein, wherein said protein comprises an amino acid sequence selected from the group consisting of:

(a) the amino acid sequence of SEQ ID NO:118;

(b) the amino acid sequence of SEQ ID NO:118 from amino acid 1 to amino acid 47;

(c) fragments of the amino acid sequence of SEQ ID NO:118, each fragment comprising eight consecutive amino acids of SEQ ID NO:118; and

(d) the amino acid sequence encoded by the cDNA insert of clone nt429_{—}1 deposited with the ATCC under accession number 98631;

the protein being substantially free from other mammalian proteins. Preferably such protein comprises the amino acid sequence of SEQ ID NO:118 or the amino acid sequence of SEQ ID NO:118 from amino acid 1 to amino acid 47. In further preferred embodiments, the present invention provides a protein comprising a fragment of the amnino acid sequence of SEQ ID NO:118 having biological activity, the fragment preferably comprising eight (more preferably twenty, most preferably thirty) consecutive amino acids of SEQ ID NO:118, or a protein comprising a fragment of the amino acid sequence of SEQ ID NO:118 having biological activity, the fragment comprising the amino acid sequence from amino acid 49 to amino acid 58 of SEQ ID NO:118.

In one embodiment, the present invention provides a composition comprising an isolated polynucleotide selected from the group consisting of:

(a) a polynucleotide comprising the nucleotide sequence of SEQ ID NO:119;

(b) a polynucleotide comprising the nucleotide sequence of SEQ ID NO:119 from nucleotide 104 to nucleotide 652;

(c) a polynucleotide comprising the nucleotide sequence of SEQ ID NO:119 from nucleotide 377 to nucleotide 652;

(d) a polynucleotide comprising the nucleotide sequence of the full-length protein coding sequence of clone pe503_{—}1 deposited with the ATCC under accession number 98631;

(e) a polynucleotide encoding the full-length protein encoded by the cDNA insert of clone pe503_{—}1 deposited with the ATCC under accession number 98631;

(f) a polynucleotide comprising the nucleotide sequence of a mature protein coding sequence of clone pe503_{—}1 deposited with the ATCC under accession number 98631;

(g) a polynucleotide encoding a mature protein encoded by the cDNA insert of clone pe503_{—}1 deposited with the ATCC under accession number 98631;

(h) a polynucleotide encoding a protein comprising the amino acid sequence of SEQ ID NO:120;

(i) a polynucleotide encoding a protein comprising a fragment of the amino acid sequence of SEQ ID NO:120 having biological activity, the fragment comprising eight consecutive amino acids of SEQ ID NO:120;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:119 from nucleotide 104tonucleotide652;thenucleotide sequence ofSEQIDNO:119 from nucleotide 377 to nucleotide 652; the nucleotide sequence of the full-length protein coding sequence of clone pe503_{—}1 deposited with the ATCC under accession number 98631; or the nucleotide sequence of a mature protein coding sequence of clone pe503_{—}1 deposited with the ATCC under accession number 98631. In other preferred embodiments, the polynucleotide encodes the full-length or a mature protein encoded by the cDNA insert of clone pe503_{—}1 deposited with the ATCC under accession number 98631. In yet other preferred embodiments, the present invention provides a polynucleotide encoding a protein comprising the amino acid sequence of SEQ ID NO:120 from amino acid 69 to amino acid 125. In further preferred embodiments, the present invention provides a polynucleotide encoding a protein comprising a fragment of the amino acid sequence of SEQ ID NO:120 having biological activity, the fragment preferably comprising eight (more preferably twenty, most preferably thirty) consecutive amino acids of SEQ ID NO:120, or a polynucleotide encoding a protein comprising a fragment of the amino acid sequence of SEQ ID NO:120 having biological activity, the fragment comprising the amino acid sequence from amino acid 86 to amino acid 95 of SEQ ID NO:120.

Other embodiments provide the gene corresponding to the cDNA sequence of SEQ ID NO:119.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQIDNO:119, butexcluding the poly(A) tail at the 3′ end of SEQ ID NO:119; and

(ab) the nucleotide sequence of the cDNA insert of clone pe503_{—}1 deposited with the ATCC under accession number 98631;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:119, but excluding the poly(A) tail at the 3′ end of SEQ ID NO:119; and

(bb) the nucleotide sequence of the cDNA insert of clone pe503_{—}1 deposited with the ATCC under accession number 98631;

(ii) hybridizing said primer(s) to human genonic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:119, and extending contiguously from a nucleotide sequence corresponding to the 5′ end of SEQ ID NO:119 to a nucleotide sequence corresponding to the 3′ end of SEQ ID NO:119, but excluding the poly(A) tail at the 3′ end of SEQ ID NO:119. Also preferably the polynucleotide isolated according to the above process comprises a nucleotide sequence corresponding to the cDNA sequence of SEQ ID NO:119 from nucleotide 104 to nucleotide 652, and extending contiguously from a nucleotide sequence corresponding to the 5′ end of said sequence of SEQ ID NO:119 from nucleotide 104 to nucleotide 652, to a nucleotide sequence corresponding to the 3′ end of said sequence of SEQ ID NO:119 from nucleotide 104 to nucleotide 652. Also preferably the polynucleotide isolated according to the above process comprises a nucleotide sequence corresponding to the cDNA sequence of SEQ ID NO:119 from nucleotide 377 to nucleotide 652, and extending contiguously from a nucleotide sequence corresponding to the 5′ end of said sequence of SEQ ID NO:119 from nucleotide 377 to nucleotide 652, to a nucleotide sequence corresponding to the 3′ end of said sequence of SEQ ID NO:119 from nucleotide 377 to nucleotide 652.

In other embodiments, the present invention provides a composition comprising a protein, wherein said protein comprises an amino acid sequence selected from the group consisting of:

(a) the anino acid sequence of SEQ ID NO:120;

(b) the amino acid sequence of SEQ ID NO:120 from amino acid 69 to amino acid 125;

(c) fragments of the amino acid sequence of SEQ ID NO:120, each fragment comprising eight consecutive amino acids of SEQ ID NO:120; and

(d) the amino acid sequence encoded by the cDNA insert of clone pe503_{—}1 deposited with the ATCC under accession number 98631;

the protein being substantially free from other mammalian proteins. Preferably such protein comprises the amino acid sequence of SEQ ID NO:120 or the amino acid sequence of SEQ ID NO:120 from amino acid 69 to amino acid 125. In further preferred embodiments, the present invention provides a protein comprising a fragment of the amino acid sequence of SEQ ID NO:120 having biological activity, the fragment preferably comprising eight (more preferably twenty, most preferably thirty) consecutive amino acids of SEQ ID NO:120, or a protein comprising a fragment of the amino acid sequence of SEQ ID NO:120 having biological activity, the fragment comprising the amino acid sequence from amino acid 86 to amino acid 95 of SEQ ID NO:120.

In one embodiment, the present invention provides a composition comprising an isolated polynucleotide selected from the group consisting of:

(a) a polynucleotide comprising the nucleotide sequence of SEQ ID NO:121;

(b) a polynucleotide comprising the nucleotide sequence of SEQ ID NO: 121 from nucleotide 23 to nucleotide 442;

(c) a polynucleotide comprising the nucleotide sequence of SEQ ID NO:121 from nucleotide 224 to nucleotide 442;

(d) a polynucleotide comprising the nucleotide sequence of the full-length protein coding sequence of clone pe834_{—}6 deposited with the ATCC under accession number 98631;

(e) a polynucleotide encoding the full-length protein encoded by the cDNA insert of clone pe834_{—}6 deposited with the ATCC under accession number 98631;

(f) a polynucleotide comprising the nucleotide sequence of a mature protein coding sequence of clone pe834_{—}6 deposited with the ATCC under accession number 98631;

(g) a polynucleotide encoding a mature protein encoded by the cDNA insert of clone pe834_{—}6 deposited with the ATCC under accession number 98631;

(h) a polynucleotide encoding a protein comprising the amino acid sequence of SEQ ID NO:122;

(i) a polynucleotide encoding a protein comprising a fragment of the amino acid sequence of SEQ ID NO:122 having biological activity, the fragment comprising eight consecutive amino acids of SEQ ID NO: 122;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:121 fromnucleotide23 tonucleotide442; thenucleotidesequence of SEQIDNO:121 from nucleotide 224 to nucleotide 442; the nucleotide sequence of the full-length protein coding sequence of clone pe834_{—}6 deposited with the ATCC under accession number 98631; or the nucleotide sequence of a mature protein coding sequence of clone pe834_{—}6 deposited with the ATCC under accession number 98631. In other preferred embodiments, the polynucleotide encodes the full-length or a mature protein encoded by the cDNA insert of clone pe834_{—}6 deposited with the ATCC under accession number 98631. In further preferred embodiments, the present invention provides a polynucleotide encoding a protein comprising a fragment of the amnino acid sequence of SEQ ID NO:122 having biological activity, the fragment preferably comprising eight (more preferably twenty, most preferably thirty) consecutive amino acids of SEQ ID NO:122, or a polynucleotide encoding a protein comprising a fragment of the amiuno acid sequence of SEQ ID NO:122 having biological activity, the fragment comprising the amnino acid sequence from amino acid 65 to amino acid 74 of SEQ ID NO:122.

Other embodiments provide the gene corresponding to the cDNA sequence of SEQ ID NO:121.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:121, but excluding the poly(A) tail at the 3′ end of SEQ ID NO:121; and

(ab) the nucleo tide sequence of the cDNA insert of clone pe834_{—}6 deposited with the ATCC under accession number 98631;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQIDNO:121,butexcludingthepoly(A)tailatthe 3′ end of SEQ ID NO:121; and

(bb) the nucleotide sequence of the cDNA insert of clone pe834_{—}6 deposited with the ATCC under accession number 98631;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:121, and extending contiguously from a nucleotide sequence corresponding to the 5′ end of SEQ ID NO:121 to a nucleotide sequence corresponding to the 3′ end of SEQ ID NO:121, but excluding the poly(A) tail at the 3′ end of SEQ ID NO:121. Also preferably the polynucleotide isolated according to the above process comprises a nucleotide sequence corresponding to the cDNA sequence of SEQ ID NO: 121 from nucleotide 23 to nucleotide 442, and extending contiguously from a nucleotide sequence corresponding to the 5′ end of said sequence of SEQ ID NO:121 from nucleotide 23 to nucleotide 442, to a nucleotide sequence corresponding to the 3′ end of said sequence of SEQ ID NO:121 from nucleotide 23 to nucleotide 442.

In other embodiments, the present invention provides a composition comprising a protein, wherein said protein comprises an amino acid sequence selected from the group consisting of:

(a) the amino acid sequence of SEQ ID NO:122;

(b) fragments of the amino acid sequence of SEQ ID NO:122, each fragment comprising eight consecutive amino acids of SEQ ID NO:122; and

(c) the amino acid sequence encoded by the cDNA insert of clone pe834_{—}6 deposited with the ATCC under accession number 98631;

the protein being substantially free from other mammalian proteins. Preferably such protein comprises the amino acid sequence of SEQ ID NO:122. In further preferred embodiments, the present invention provides a protein comprising a fragment of the amino acid sequence of SEQ ID NO:122 having biological activity, the fragment preferably comprising eight (more preferably twenty, most preferably thirty) consecutive amino acids of SEQ ID NO:122, or a protein comprising a fragment of the amino acid sequence of SEQ ID NO:122 having biological activity, the fragment comprising the amino acid sequence from amino acid 65 to amino acid 74 of SEQ ID NO:122.

In one embodiment, the present invention provides a composition comprising an isolated polynucleotide selected from the group consisting of:

(a) a polynucleotide comprising the nucleotide sequence of SEQ ID NO:123;

(b) a polynucleotide comprising the nucleotide sequence of SEQ ID NO: 123 from nucleotide 98 to nucleotide 265;

(c) a polynucleotide comprising the nucleotide sequence of SEQ ID NO:123 from nucleotide 152 to nucleotide 265;

(d) a polynucleotide comprising the nucleotide sequence of the full-length protein coding sequence of clone ya10_{—}1 deposited with the ATCC under accession number 98631;

(e) a polynucleotide encoding the full-length protein encoded by the cDNA insert of clone ya10_{—}1 deposited with the ATCC under accession number 98631;

(f) a polynucleotide comprising the nucleotide sequence of a mature protein coding sequence of clone ya10_{—}1 deposited with the ATCC under accession number 98631;

(g) a polynucleotide encoding a mature protein encoded by the cDNA insert of clone ya10_{—}1 deposited with the ATCC under accession number 98631;

(h) a polynucleotide encoding a protein comprising the amino acid sequence of SEQ ID NO:124;

(i) a polynucleotide encoding a protein comprising a fragment of the amino acid sequence of SEQ ID NO:124 having biological activity, the fragment comprising eight consecutive amino acids of SEQ ID NO:124;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:123 from nucleotide 98 to nucleotide 265; the nucleotide sequence of SEQ ID NO:123 from nucleotide 152 to nucleotide 265; the nucleotide sequence of the full-length protein coding sequence of clone ya10_{—}1 deposited with the ATCC under accession number 98631; or the nucleotide sequence of a mature protein coding sequence of clone ya10_{—}1 deposited with the ATCC under accession number 98631. In other preferred embodiments, the polynucleotide encodes the full-length or a mature protein encoded by the cDNA insert of clone ya10_{—}1 deposited with the ATCC under accession number 98631.

In further preferred embodiments, the present invention provides a polynucleotide encoding a protein comprising a fragment of the amino acid sequence of SEQ ID NO:124 having biological activity, the fragment preferably comprising eight (more preferably twenty, most preferably thirty) consecutive amino acids of SEQ ID NO:124, or a polynucleotide encoding a protein comprising a fragment of the amino acid sequence of SEQ ID NO:124 having biological activity, the fragment comprising the amino acid sequence from amino acid 22 to amino acid 31 of SEQ ID NO:124.

Other embodiments provide the gene corresponding to the cDNA sequence of SEQ ID NO:123.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:123, but excluding the poly(A) tail at the 3′ end of SEQ ID NO:123; and

(ab) the nucleotide sequence of the cDNA insert of clone ya10_{—}1 deposited with the ATCC under accession number 98631;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO: 123, but excluding the poly(A) tail at the 3′ end of SEQ ID NO:123; and

(bb) the nucleotide sequence of the cDNA insert of clone ya10_{—}1 deposited with the ATCC under accession number 98631;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:123, and extending contiguously from a nucleotide sequence corresponding to the 5′ end of SEQ ID NO:123 to a nucleotide sequence corresponding to the 3′ end of SEQ ID NO:123, but excluding the poly(A) tail at the 3′ end of SEQ ID NO:123. Also preferably the polynucleotide isolated according to the above process comprises a nucleotide sequence corresponding to the cDNA sequence of SEQ ID NO:123 from nucleotide 98 to nucleotide 265, and extending contiguously from a nucleotide sequence corresponding to the 5′ end of said sequence of SEQ ID NO:123 from nucleotide 98 to nucleotide 265, to a nucleotide sequence corresponding to the 3′ end of said sequence of SEQ ID NO:123 from nucleotide 98 to nucleotide 265. Also preferably the polynucleotide isolated according to the above process comprises a nucleotide sequence corresponding to the cDNA sequence of SEQ ID NO:123 from nucleotide 152 to nucleotide 265, and extending contiguously from a nucleotide sequence corresponding to the 5′ end of said sequence of SEQ ID NO:123 from nucleotide 152 to nucleotide 265, to a nucleotide sequence corresponding to the 3′ end of said sequence of SEQ ID NO: 123 from nucleotide 152 to nucleotide 265.

In other embodiments, the present invention provides a composition comprising a protein, wherein said protein comprises an amino acid sequence selected from the group consisting of:

(a) the amino acid sequence of SEQ ID NO:124;

(b) fragments of the amino acid sequence of SEQ ID NO:124, each fragment comprising eight consecutive amino acids of SEQ ID NO:124; and

(c) the amino acid sequence encoded by the cDNA insert of clone ya10_{—}1 deposited with the ATCC under accession number 98631;

the protein being substantially free from other mammalian proteins. Preferably such protein comprises the amino acid sequence of SEQ ID NO:124. In further preferred embodiments, the present invention provides a protein comprising a fragment of the amino acid sequence of SEQ ID NO:124 having biological activity, the fragment preferably comprising eight (more preferably twenty, most preferably thirty) consecutive amino acids of SEQ ID NO:124, or a protein comprising a fragment of the amino acid sequence of SEQ ID NO:124 having biological activity, the fragment comprising the amino acid sequence from amino acid 22 to amino acid 31 of SEQ ID NO:124.

In one embodiment, the present invention provides a composition comprising an isolated polynucleotide selected from the group consisting of:

(a) a polynucleotide comprising the nucleotide sequence of SEQ ID NO:125;

(b) a polynucleotide comprising the nucleotide sequence of SEQ ID NO: 125 from nucleotide 176 to nucleotide 583;

(c) a polynucleotide comprising the nucleotide sequence of the full-length protein coding sequence of clone yb40_{—}1 deposited with the ATCC under accession number 98631;

(d) a polynucleotide encoding the full-length protein encoded by the cDNA insert of clone yb40_{—}1 deposited with the ATCC under accession number 98631;

(e) a polynucleotide comprising the nucleotide sequence of a mature protein coding sequence of clone yb40_{—}1 deposited with the ATCC under accession number 98631;

(f) a polynucleotide encoding a mature protein encoded by the cDNA insert of clone yb40_{—}1 deposited with the ATCC under accession number 98631;

(g) a polynucleotide encoding a protein comprising the amino acid sequence of SEQ ID NO:126;

(h) a polynucleotide encoding a protein comprising a fragment of the amino acid sequence of SEQ ID NO:126 having biological activity, the fragment comprising eight consecutive amino acids of SEQ ID NO:126;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:125 from nucleotide 176 to nucleotide 583; the nucleotide sequence of the full-length protein coding sequence of clone yb40_{—}1 deposited with the ATCC under accession number 98631; or the nucleotide sequence of a mature protein coding sequence of clone yb40_{—}1 deposited with the ATCC under accession number 98631. In other preferred embodiments, the polynucleotide encodes the full-length or a mature protein encoded by the cDNA insert of clone yb40_{—}1 deposited with the ATCC under accession number 98631. In further preferred embodiments, the present invention provides a polynucleotide encoding a protein comprising a fragment of the amino acid sequence of SEQ ID NO:126 having biological activity, the fragment preferably comprising eight (more preferably twenty, most preferably thirty) consecutive amino acids of SEQ ID NO:126, or a polynucleotide encoding a protein comprising a fragment of the amino acid sequence of SEQ ID NO:126 having biological activity, the fragment comprising the amino acid sequence from amino acid 63 to amLino acid 72 of SEQ ID NO:126.

Other embodiments provide the gene corresponding to the cDNA sequence of SEQ ID NO:125.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQIDNO:125,but excluding the poly(A) tail at the 3′ end of SEQ ID NO:125; and

(ab) the nucleotide sequence of the cDNA insert of clone yb40_{—}1 deposited with the ATCC under accession number 98631;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:125, but excluding the poly(A) tail at the 3′ end of SEQ ID NO:125; and

(bb) the nucleotide sequence of the cDNA insert of clone yb40_{—}1 deposited with the ATCC under accession number 98631;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:125, and extending contiguously from a nucleotide sequence corresponding to the 5′ end of SEQ ID NO:125 to a nucleotide sequence corresponding to the 3′ end of SEQ ID NO:125, but excluding the poly(A) tail at the 3′ end of SEQ ID NO:125. Also preferably the polynucleotide isolated according to the above process comprises a nucleotide sequence corresponding to the cDNA sequence of SEQ ID NO:125 from nucleotide 176 to nucleotide 583, and extending contiguously from a nucleotide sequence corresponding to the 5′ end of said sequence of SEQ ID NO:125 from nucleotide 176 to nucleotide 583, to a nucleotide sequence corresponding to the 3′ end of said sequence of SEQ ID NO:125 from nucleotide 176 to nucleotide 583.

In other embodiments, the present invention provides a composition comprising a protein, wherein said protein comprises an amino acid sequence selected from the group consisting of:

(a) the amino acid sequence of SEQ ID NO:126;

(b) fragments of the amino acid sequence of SEQ ID NO:126, each fragment comprising eight consecutive amino acids of SEQ ID NO:126; and

(c) the amino acid sequence encoded by the cDNA insert of clone yb40_{—}1 deposited with the ATCC under accession number 98631;

the protein being substantially free from other mammalian proteins. Preferably such protein comprises the amino acid sequence of SEQ ID NO:126. In further preferred embodiments, the present invention provides a protein comprising a fragment of the amino acid sequence of SEQ ID NO:126 having biological activity, the fragment preferably comprising eight (more preferably twenty, most preferably thirty) consecutive amino acids of SEQ ID NO:126, or a protein comprising a fragment of the amino acid sequence of SEQ ID NO:126 having biological activity, the fragment comprising the amino acid sequence from amino acid 63 to amino acid 72 of SEQ ID NO:126.

In one embodiment, the present invention provides a composition comprising an isolated polynucleotide selected from the group consisting of:

(a) a polynucleotide comprising the nucleotide sequence of SEQ ID NO:127;

(b) a polynucleotide comprising the nucleotide sequence of SEQ ID NO:127 from nucleotide 734 to nucleotide 1873;

(c) a polynucleotide comprising the nucleotide sequence of SEQ ID NO:127 from nucleotide 1403 to nucleotide 1873;

(d) a polynucleotide comprising the nucleotide sequence of the full-length protein coding sequence of clone cs756_{—}2 deposited with the ATCC under accession number 98636;

(e) a polynucleotide encoding the full-length protein encoded by the cDNA insert of clone cs756_{—}2 deposited with the ATCC under accession number 98636;

(f) a polynucleotide comprising the nucleotide sequence of a mature protein coding sequence of clone cs756_{—}2 deposited with the ATCC under accession number 98636;

(g) a polynucleotide encoding a mature protein encoded by the cDNA insert of clone cs756_{—}2 deposited with the ATCC under accession number 98636;

(h) a polynucleotide encoding a protein comprising the amino acid sequence of SEQ ID NO:128;

(i) a polynucleotide encoding a protein comprising a fragment of the amino acid sequence of SEQ ID NO:128 having biological activity, the fragment comprising eight consecutive amino acids of SEQ ID NO:128;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:127 from nucleotide 734 to nucleotide 1873; the nucleotide sequence of SEQ ID NO:127 from nucleotide 1403 to nucleotide 1873; the nucleotide sequence of the full-length protein coding sequence of clone cs756_{—}2 deposited with the ATCC under accession number 98636; or the nucleotide sequence of a mature protein coding sequence of clone cs756_{—}2 deposited with the ATCC under accession number 98636. In other preferred embodiments, the polynucleotide encodes the full-length or a mature protein encoded by the cDNA insert of clone cs756_{—}2 deposited with the ATCC under accession number 98636. In further preferred embodiments, the present invention provides a polynucleotide encoding a protein comprising a fragment of the amino acid sequence of SEQ ID NO:128 having biological activity, the fragment preferably comprising eight (more preferably twenty, most preferably thirty) consecutive amino acids of SEQ ID NO:128, or a polynucleotide encoding a protein comprising a fragment of the amino acid sequence of SEQ ID NO:128 having biological activity, the fragment comprising the amino acid sequence from amino acid 185 to amino acid 194 of SEQ ID NO:128.

Other embodiments provide the gene corresponding to the cDNA sequence of SEQ ID NO:127.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:127, but excluding the poly(A) tail at the 3′ end of SEQ ID NO:127; and

(ab) the nucleotide sequence of the cDNA insert of clone cs756_{—}2 deposited with the ATCC under accession number 98636;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:127, but excluding the poly(A) tail at the 3′ end of SEQ ID NO:127; and

(bb) the nucleotide sequence of the cDNA insert of clone cs756_{—}2 deposited with the ATCC under accession number 98636;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:127, and extending contiguously from a nucleotide sequence corresponding to the 5′ end of SEQ ID NO:127 to a nucleotide sequence corresponding to the 3′ end of SEQ ID NO:127, but excluding the poly(A) tail at the 3′ end of SEQ ID NO:127. Also preferably the polynucleotide isolated according to the above process comprises a nucleotide sequence corresponding to the cDNA sequence of SEQ ID NO:127 from nucleotide 734 to nucleotide 1873, and extending contiguously from a nucleotide sequence corresponding to the 5′ end of said sequence of SEQ ID NO:127 from nucleotide 734 to nucleotide 1873, to a nucleotide sequence corresponding to the 3′ end of said sequence of SEQ ID NO:127 from nucleotide 734 to nucleotide 1873. Also preferably the polynucleotide isolated according to the above process comprises a nucleotide sequence corresponding to the cDNA sequence of SEQ ID NO:127 from nucleotide 1403 to nucleotide 1873, and extending contiguously from a nucleotide sequence corresponding to the 5′ end of said sequence of SEQ ID NO:127 from nucleotide 1403 to nucleotide 1873, to a nucleotide sequence corresponding to the 3′ end of said sequence of SEQ ID NO:127 from nucleotide 1403 to nucleotide 1873.

In other embodiments, the present invention provides a composition comprising a protein, wherein said protein comprises an amino acid sequence selected from the group consisting of:

(a) the amino acid sequence of SEQ ID NO:128;

(b) fragments of the amino acid sequence of SEQ ID NO:128, each fragment comprising eight consecutive amino acids of SEQ ID NO:128; and

(c) the amino acid sequence encoded by the cDNA insert of clone cs756_{—}2 deposited with the ATCC under accession number 98636;

the protein being substantially free from other mammalian proteins. Preferably such protein comprises the amino acid sequence of SEQ ID NO:128. In further preferred embodiments, the present invention provides a protein comprising a fragment of the amino acid sequence of SEQ ID NO:128 having biological activity, the fragment preferably comprising eight (more preferably twenty, mostpreferably thirty) consecutive amino acids of SEQ ID NO:128, or a protein comprising a fragment of the arino acid sequence of SEQ ID NO:128 having biological activity, the fragment comprising the amino acid sequence from amino acid 185 to amnino acid 194 of SEQ ID NO:128.

In one embodiment, the present invention provides a composition comprising an isolated polynucleotide selected from the group consisting of:

(a) a polynucleotide comprising the nucleotide sequence of SEQ ID NO:129;

(b) a polynucleotide comprising the nucleotide sequence of SEQ ID NO:129 from nucleotide 26 to nucleotide 1738;

(c) a polynucleotide comprising the nucleotide sequence of SEQ ID NO:129 from nucleotide 140 to nucleotide 1738;

(d) a polynucleotide comprising the nucleotide sequence of the full-length protein coding sequence of clone ew150_{—}1 deposited with the ATCC under accession number 98636;

(e) a polynucleotide encoding the full-length protein encoded by the cDNA insert of clone ew150_{—}1 deposited with the ATCC under accession number 98636;

(f) a polynucleotide comprising the nucleotide sequence of a mature protein coding sequence of clone ew150_{—}1 deposited with the ATCC under accession number 98636;

(g) a polynucleotide encoding a mature protein encoded by the cDNA insert of clone ew150_{—}1 deposited with the ATCC under accession number 98636;

(h) a polynucleotide encoding a protein comprising the amino acid sequence of SEQ ID NO:130;

(i) a polynucleotide encoding a protein comprising a fragment of the amino acid sequence of SEQ ID NO:130 having biological activity, the fragment comprising eight consecutive amino acids of SEQ ID NO:130;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:129 from nucleotide 26 to nucleotide 1738; the nucleotide sequence of SEQ ID NO:129 from nucleotide 140to nucleotide 1738; thenucleotidesequence of the full length protein coding sequence of clone ew150_{—}1 deposited with the ATCC under accession number 98636; or the nucleotide sequence of a mature protein coding sequence of clone ew150_{—}1 deposited with the ATCC under accession number 98636. In other preferred embodiments, the polynucleotide encodes the full-length or a mature protein encoded by the cDNA insert of clone ew150_{—}1 deposited with the ATCC under accession number 98636. In yet other preferred embodiments, the present invention provides a polynucleotide encoding a protein comprising the amino acid sequence of SEQ ID NO:130 from amino acid 108 to amino acid 166. In further preferred embodiments, the present invention provides a polynucleotide encoding a protein comprising a fragment of the amino acid sequence of SEQ ID NO:130 having biological activity, the fragment preferably comprising eight (more preferably twenty, most preferably thirty) consecutive amino acids of SEQ ID NO:130, or a polynucleotide encoding a protein comprising a fragment of the amino acid sequence of SEQ ID NO:130 having biological activity, the fragment comprising the amino acid sequence from amino acid 280 to amino acid 289 of SEQ ID NO:130.

Other embodiments provide the gene corresponding to the cDNA sequence of SEQ ID NO:129.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ IDNO:129, but excluding the poly(A) tail at the 3′ end of SEQ ID NO:129; and

(ab) the nucleotide sequence of the cDNA insert of clone ew150_{—}1 deposited with the ATCC under accession number 98636;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:129, but excluding the poly(A) tail at the 3′ end of SEQ ID NO:129; and

(bb) the nucleotide sequence of the cDNA insert of clone ew150_{—}1 deposited with the ATCC under accession number 98636;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:129, and extending contiguously from a nucleotide sequence corresponding to the 5′ end of SEQ ID NO:129 to a nucleotide sequence corresponding to the 3′ end of SEQ ID NO:129, but excluding the poly(A) tail at the 3′ end of SEQ ID NO:129. Also preferably the polynucleotide isolated according to the above process comprises a nucleotide sequence corresponding to the cDNA sequence of SEQ ID NO:129 from nucleotide 26 to nucleotide 1738, and extending contiguously from a nucleotide sequence corresponding to the 5′ end of said sequence of SEQ ID NO:129 from nucleotide 26 to nucleotide 1738, to a nucleotide sequence corresponding to the 3′ end of said sequence of SEQ ID NO:129 from nucleotide 26 to nucleotide 1738. Also preferably the polynucleotide isolated according to the above process comprises a nucleotide sequence corresponding to the cDNA sequence of SEQ ID NO:129 from nucleotide 140 to nucleotide 1738, and extending contiguously from a nucleotide sequence corresponding to the 5′ end of said sequence of SEQ ID NO:129 from nucleotide 140 to nucleotide 1738, to a nucleotide sequence corresponding to the 3′ end of said sequence of SEQ ID NO:129 from nucleotide 140 to nucleotide 1738.

In other embodiments, the present invention provides a composition comprising a protein, wherein said protein comprises an amino acid sequence selected from the group consisting of:

(a) the amino acid sequence of SEQ ID NO:130;

(b) the amino acid sequence of SEQ ID NO:130 from amino acid 108 to amino acid 166;

(c) fragments of the amino acid sequence of SEQ ID NO:130, each fragment comprising eight consecutive amino acids of SEQ ID NO:130; and

(d) the amino acid sequence encoded by the cDNA insert of clone ew150_{—}1 deposited with the ATCC under accession number 98636;

the protein being substantially free from other mammalian proteins. Preferably such protein comprises the amino acid sequence of SEQ ID NO:130 or the amino acid sequence of SEQ ID NO:130 from amino acid 108 to amino acid 166. In further preferred embodiments, the present invention provides a protein comprising a fragment of the amino acid sequence of SEQ ID NO:130 having biological activity, the fragment preferably comprising eight (more preferably twenty, most preferably thirty) consecutive amino acids of SEQ ID NO:130, or a protein comprising a fragment of the amino acid sequence of SEQ ID NO:130 having biological activity, the fragment comprising the amino acid sequence from amino acid 280 to amino acid 289 of SEQ ID NO:130.

In one embodiment, the present invention provides a composition comprising an isolated polynucleotide selected from the group consisting of:

(a) a polynucleotide comprising the nucleotide sequence of SEQ ID NO:131;

(b) a polynucleotide comprising the nucleotide sequence of SEQ ID NO:131 from nucleotide 1101 to nucleotide 1910;

(c) a polynucleotide comprising the nucleotide sequence of SEQ ID NO:131 from nucleotide 1260 to nucleotide 1910;

(d) a polynucleotide comprising the nucleotide sequence of the full-length protein coding sequence of clone gg894_{—}13 deposited with the ATCC under accession number 98636;

(e) a polynucleotide encoding the full-length protein encoded by the cDNA insert of clone gg894_{—}13 deposited with the ATCC under accession number 98636;

(f) a polynucleotide comprising the nucleotide sequence of a mature protein coding sequence of clone gg894_{—}13 deposited with the ATCC under accession number 98636;

(g) a polynucleotide encoding a mature protein encoded by the cDNA insert of clone gg894_{—}13 deposited with the ATCC under accession number 98636;

(h) a polynucleotide encoding a protein comprising the amino acid sequence of SEQ ID NO:132;

(i) a polynucleotide encoding a protein comprising a fragment of the amino acid sequence of SEQ ID NO:132 having biological activity, the fragment comprising eight consecutive amino acids of SEQ ID NO:132;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:131 from nucleotide 1101 to nucleotide 1910; the nucleotide sequence of SEQ ID NO:131 from nucleotide 1260 to nucleotide 1910; the nucleotide sequence of the full-length protein coding sequence of clone gg894_{—}13 deposited with the ATCC under accession number 98636; or the nucleotide sequence of a mature protein coding sequence of clone gg894_{—}13 deposited with the ATCC under accession number 98636. In other preferred embodiments, the polynucleotide encodes the full-length or a mature protein encoded by the cDNA insert of clone gg894_{—}13 deposited with the ATCC under accession number 98636. In further preferred embodiments, the present invention provides a polynucleotide encoding a protein comprising a fragment of the amino acid sequence of SEQ ID NO:132 having biological activity, the fragment preferably comprising eight (more preferably twenty, most preferably thirty) consecutive amino acids of SEQ ID NO:132, or a polynucleotide encoding a protein comprising a fragment of the amino acid sequence of SEQ ID NO:132 having biological activity, the fragment comprising the amino acid sequence from amnino acid 130 to amino acid 139 of SEQ ID NO: 132.

Other embodiments provide the gene corresponding to the cDNA sequence of SEQ ID NO:131.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:131, but excluding the poly(A) tail at the 3′ end of SEQ ID NO:131; and

(ab) the nucleotide sequence of the cDNA insert of clone gg894_{—}13 deposited with the ATCC under accession number 98636;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:131, but excluding the poly(A) tail at the 3′ end of SEQ ID NO:131; and

(bb) the nucleotide sequence of the cDNA insert of clone gg894_{—}13 deposited with the ATCC under accession number 98636;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:131, and extending contiguously from a nucleotide sequence corresponding to the 5′ end of SEQ ID NO:131 to a nucleotide sequence corresponding to the 3′ end of SEQ ID NO:131, but excluding the poly(A) tail at the 3′ end of SEQ ID NO:131. Also preferably the polynucleotide isolated according to the above process comprises a nucleotide sequence corresponding to the cDNA sequence of SEQ ID NO:131 from nucleotide 1101 to nucleotide 1910, and extending contiguously from a nucleotide sequence corresponding to the 5′ end of said sequence of SEQ ID NO: 131 from nucleotide 1101 to nucleotide 1910, to a nucleotide sequence corresponding to the 3′ end of said sequence of SEQ ID NO:131 from nucleotide 1101 to nucleotide 1910. Also preferably the polynucleotide isolated according to the above process comprises a nucleotide sequence corresponding to the cDNA sequence of SEQ ID NO:131 from nucleotide 1260 to nucleotide 1910, and extending contiguously from a nucleotide sequence corresponding to the 5′ end of said sequence of SEQ ID NO:131 from nucleotide 1260 to nucleotide 1910, to a nucleotide sequence corresponding to the 3′ end of said sequence of SEQ ID NO:131 from nucleotide 1260 to nucleotide 1910.

In other embodiments, the present invention provides a composition comprising a protein, wherein said protein comprises an amino acid sequence selected from the group consisting of:

(a) the amino acid sequence of SEQ ID NO:132;

(b) fragments of the amino acid sequence of SEQ ID NO:132, each fragment comprising eight consecutive amino acids of SEQ ID NO:132; and

(c) the amino acid sequence encoded by the cDNA insert of clone gg894_{—}13 deposited with the ATCC under accession number 98636;

the protein being substantially free from other mammalian proteins. Preferably such protein comprises the amino acid sequence of SEQ ID NO:132. In further preferred embodiments, the present invention provides a protein comprising a fragment of the amino acid sequence of SEQ ID NO:132 having biological activity, the fragment preferably comprising eight (more preferably twenty, most preferably thirty) consecutive amino acids of SEQ ID NO:132, or a protein comprising a fragment of the amino acid sequence of SEQ ID NO:132 having biological activity, the fragment comprising the amino acid sequence from amino acid 130 to amino acid 139 of SEQ ID NO: 132.

In one embodiment, the present invention provides a composition comprising an isolated polynucleotide selected from the group consisting of:

(a) a polynucleotide comprising the nucleotide sequence of SEQ ID NO:133;

(b) a polynucleotide comprising the nucleotide sequence of SEQ ID NO:133 from nucleotide 452 to nucleotide 1102;

(c) a polynucleotide comprising the nucleotide sequence of the full-length protein coding sequence of clone it217_{—}2 deposited with the ATCC under accession number 98636;

(d) a polynucleotide encoding the full-length protein encoded by the cDNA insert of clone it217_{—}2 deposited with the ATCC under accession number 98636;

(e) a polynucleotide comprising the nucleotide sequence of a mature protein coding sequence of clone it217_{—}2 deposited with the ATCC under accession number 98636;

(f) a polynucleotide encoding a mature protein encoded by the cDNA insert of clone it217_{—}2 deposited with the ATCC under accession number 98636;

(g) a polynucleotide encoding a protein comprising the amino acid sequence of SEQ ID NO:134;

(h) a polynucleotide encoding a protein comprising a fragment of the amino acid sequence of SEQ ID NO:134 having biological activity, the fragment comprising eight consecutive amino acids of SEQ ID NO:134;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:133 from nucleotide 452 to nucleotide 1102; the nucleotide sequence of the full-length protein coding sequence of clone it217_{—}2 deposited with the ATCC under accession number 98636; or the nucleotide sequence of a mature protein coding sequence of clone it217_{—}2 deposited with the ATCC under accession number 98636. In other preferred embodiments, the polynucleotide encodes the full-length or a mature protein encoded by the cDNA insert of clone it217_{—}2 deposited with the ATCC under accession number 98636. In further preferred embodiments, the present invention provides a polynucleotide encoding a protein comprising a fragment of the amino acid sequence of SEQ ID NO:134 having biological activity, the fragment preferably comprising eight (more preferably twenty, most preferably thirty) consecutive amino acids of SEQ ID NO:134, or a polynucleotide encoding a protein comprising a fragment of the amino acid sequence of SEQ ID NO:134 having biological activity, the fragment comprising the amino acid sequence from amino acid 103 to amino acid 112 of SEQ ID NO:134.

Other embodiments provide the gene corresponding to the cDNA sequence of SEQ ID NO:133.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:133, but excluding the poly(A) tail at the 3′ end of SEQ ID NO:133; and

(ab) the nucleotide sequence of the cDNA insert of clone it217_{—}2 deposited with the ATCC under accession number 98636;

(ii) hybridizing said probe(s) to human genormic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:133, but excluding the poly(A) tail at the 3′ end of SEQ ID NO:133; and

(bb) the nucleotide sequence of the cDNA insert of clone it217_{—}2 deposited with the ATCC under accession number 98636;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:133, and extending contiguously from a nucleotide sequence corresponding to the 5′ end of SEQ ID NO:133 to a nucleotide sequence corresponding to the 3′ end of SEQ ID NO:133, but excluding the poly(A) tail at the 3′ end of SEQ ID NO:133. Also preferably the polynucleotide isolated according to the above process comprises a nucleotide sequence corresponding to the cDNA sequence of SEQ ID NO:133 from nucleotide 452 to nucleotide 1102, and extending contiguously from a nucleotide sequence corresponding to the 5′ end of said sequence of SEQ ID NO:133 from nucleotide 452 to nucleotide 1102, to a nucleotide sequence corresponding to the 3′ end of said sequence of SEQ ID NO:133 from nucleotide 452 to nucleotide 1102.

In other embodiments, the present invention provides a composition comprising a protein, wherein said protein comprises an amino acid sequence selected from the group consisting of:

(a) the amnino acid sequence of SEQ ID NO:134;

(b) fragments of the amino acid sequence of SEQ ID NO:134, each fragment comprising eight consecutive amino acids of SEQ ID NO:134; and

(c) the amino acid sequence encoded by the cDNA insert of clone it217_{—}2 deposited with the ATCC under accession number 98636;

the protein being substantially free from other mammalian proteins. Preferably such protein comprises the amino acid sequence of SEQ ID NO:134. In further preferred embodiments, the present invention provides a protein comprising a fragment of the amino acid sequence of SEQ ID NO:134 having biological activity, the fragment preferably comprising eight (more preferably twenty, most preferably thirty) consecutive amino acids of SEQ ID NO:134, or a protein comprising a fragment of the amino acid sequence of SEQ ID NO:134 having biological activity, the fragment comprising the amino acid sequence from amino acid 103 to amino acid 112 of SEQ ID NO:134.

In one embodiment, the present invention provides a composition comprising an isolated polynucleotide selected from the group consisting of:

(a) a polynucleotide comprising the nucleotide sequence of SEQ ID NO:135;

(b) a polynucleotide comprising the nucleotide sequence of SEQ ID NO:135 from nucleotide 127 to nucleotide 387;

(c) a polynucleotide comprising the nucleotide sequence of SEQ ID NO:135 from nucleotide 172 to nucleotide 387;

(d) a polynucleotide comprising the nucleotide sequence of the full-length protein coding sequence of clone ml235_{—}2 deposited with the ATCC under accession number 98636;

(e) a polynucleotide encoding the full-length protein encoded by the cDNA insert of clone ml235_{—}2 deposited with the ATCC under accession number 98636;

(f) a polynucleotide comprising the nucleotide sequence of a mature protein coding sequence of clone ml235_{—}2 deposited with the ATCC under accession number 98636;

(g) a polynucleotide encoding a mature protein encoded by the cDNA insert of clone ml235_{—}2 deposited with the ATCC under accession number 98636;

(h) a polynucleotide encoding a protein comprising the amino acid sequence of SEQ ID NO:136;

(i) a polynucleotide encoding a protein comprising a fragment of the armino acid sequence of SEQ ID NO:136 having biological activity, the fragment comprising eight consecutive amino acids of SEQ ID NO:136;

(i) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:135 from nucleotide 127 to nucleotide 387; the nucleotide sequence of SEQ ID NO:135 from nucleotide 172 to nucleotide 387; the nucleotide sequence of the full-length protein coding sequence of clone ml235_{—}2 deposited with the ATCC under accession number 98636; or the nucleotide sequence of a mature protein coding sequence of clone ml235_{—}2 deposited with the ATCC under accession number 98636. In other preferred embodiments, the polynucleotide encodes the full-length or a mature protein encoded by the cDNA insert of clone ml235_{—}2 deposited with the ATCC under accession number 98636. In further preferred embodiments, the present invention provides a polynucleotide encoding a protein comprising a fragment of the amino acid sequence of SEQ ID NO:136 having biological activity, the fragment preferably comprising eight (more preferably twenty, most preferably thirty) consecutive amino acids of SEQ ID NO:136, or a polynucleotide encoding a protein comprising a fragment of the amino acid sequence of SEQ ID NO:136 having biological activity, the fragment comprising the amino acid sequence from amino acid 38 to amino acid 47 of SEQ ID NO:136.

Other embodiments provide the gene corresponding to the cDNA sequence of SEQ ID NO:135.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:135, but excluding the poly(A) tail at the 3′ end of SEQ ID NO:135; and

(ab) the nucleotide sequence of the cDNA insert of clone ml235_{—}2 deposited with the ATCC under accession number 98636;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:135, but excluding thepoly(A) tail at the 3′ end of SEQ ID NO:135; and

(bb) the nucleotide sequence of the cDNA insert of clone ml235_{—}2 deposited with the ATCC under accession number 98636;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:135, and extending contiguously from a nucleotide sequence corresponding to the 5′ end of SEQ ID NO:135 to a nucleotide sequence corresponding to the 3′ end of SEQ ID NO:135, but excluding the poly(A) tail at the 3′ end of SEQ ID NO:135. Also preferably the polynucleotide isolated according to the above process comprises a nucleotide sequence corresponding to the cDNA sequence of SEQ ID NO:135 from nucleotide 127 to nucleotide 387, and extending contiguously from a nucleotide sequence corresponding to the 5′ end of said sequence of SEQ ID NO:135 from nucleotide 127 to nucleotide 387, to a nucleotide sequence corresponding to the 3′ end of said sequence of SEQ ID NO:135 from nucleotide 127 to nucleotide 387. Also preferably the polynucleotide isolated according to the above process comprises a nucleotide sequence corresponding to the cDNA sequence of SEQ ID NO:135 from nucleotide 172 to nucleotide 387, and extending contiguously from a nucleotide sequence corresponding to the 5′ end of said sequence of SEQ ID NO:135 from nucleotide 172 to nucleotide 387, to a nucleotide sequence corresponding to the 3′ end of said sequence of SEQ ID NO:135 from nucleotide 172 to nucleotide 387.

In other embodiments, the present invention provides a composition comprising a protein, wherein said protein comprises an amino acid sequence selected from the group consisting of:

(a) the amino acid sequence of SEQ ID NO:136;

(b) fragments of the amino acid sequence of SEQ ID NO:136, each fragment comprising eight consecutive amino acids of SEQ ID NO:136; and

(c) the amino acid sequence encoded by the cDNA insert of clone ml235_{—}2 deposited with the ATCC under accession number 98636;

the protein being substantially free from other mammalian proteins. Preferably such protein comprises the amino acid sequence of SEQ ID NO:136. In further preferred embodiments, the present invention provides a protein comprising a fragment of the amino acid sequence of SEQ ID NO:136 having biological activity, the fragment preferably comprising eight (more preferably twenty, most preferably thirty) consecutive amino acids of SEQ ID NO:136, or a protein comprising a fragment of the amino acid sequence of SEQ ID NO:136 having biological activity, the fragment comprising the amino acid sequence from amino acid 38 to amino acid 47 of SEQ ID NO:136.

In one embodiment, the present invention provides a composition comprising an isolated polynucleotide selected from the group consisting of:

(a) a polynucleotide comprising the nucleotide sequence of SEQ ID NO:137;

(b) a polynucleotide comprising the nucleotide sequence of SEQ ID NO:137 from nucleotide 147 to nucleotide 1163;

(c) a polynucleotide comprising the nucleotide sequence of SEQ ID NO:137 from nucleotide 273 to nucleotide 1163;

(d) a polynucleotide comprising the nucleotide sequence of the full-length protein coding sequence of clone mt24_{—}2 deposited with the ATCC under accession number 98636;

(e) a polynucleotide encoding the full-length protein encoded by the cDNA insert of clone mt24_{—}2 deposited with the ATCC under accession number 98636;

(f) a polynucleotide comprising the nucleotide sequence of a mature protein coding sequence of clone mt24_{—}2 deposited with the ATCC under accession number 98636;

(g) a polynucleotide encoding a mature protein encoded by the cDNA insert of clone mt24_{—}2 deposited with the ATCC under accession number 98636;

(h) a polynucleotide encoding a protein comprising the amino acid sequence of SEQ ID NO:138;

(i) a polynucleotide encoding a protein comprising a fragment of the amino acid sequence of SEQ ID NO:138 having biological activity, the fragment comprising eight consecutive amino acids of SEQ ID NO:138;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:137 from nucleotide 147 to nucleotide 1163; the nucleotide sequence of SEQ ID NO:137 from nucleotide 273 to nucleotide 1163; the nucleotide sequence of the full-length protein coding sequence of clone mt24_{—}2 deposited with the ATCC under accession number 98636; or the nucleotide sequence of a mature protein coding sequence of clone mt24_{—}2 deposited with the ATCC under accession number 98636. In other preferred embodiments, the polynucleotide encodes the full-length or a mature protein encoded by the cDNA insert of clone mt24_{—}2 deposited with the ATCC under accession number 98636. In further preferred embodiments, the present invention provides a polynucleotide encoding a protein comprising a fragment of the amino acid sequence of SEQ ID NO:138 having biological activity, the fragment preferably comprising eight (more preferably twenty, most preferably thirty) consecutive amino acids of SEQ ID NO:138, or a polynucleotide encoding a protein comprising a fragment of the amino acid sequence of SEQ ID NO:138 having biological activity, the fragment comprising the amino acid sequence from amino acid 164 to amino acid 173 of SEQ ID NO:138.

Other embodiments provide the gene corresponding to the cDNA sequence of SEQ ID NO:137.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:137, but excluding the poly(A) tail at the 3′ end of SEQ ID NO:137; and

(ab) the nucleotide sequence of the cDNA insert of clone mt24_{—}2 deposited with the ATCC under accession number 98636;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:137, but excluding the poly(A) tail at the 3′ end of SEQ ID NO:137; and

(bb) the nucleotide sequence of the cDNA insert of clone mt24_{—}2 deposited with the ATCC under accession number 98636;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:137, and extending contiguously from a nucleotide sequence corresponding to the 5′ end of SEQ ID NO:137 to a nucleotide sequence corresponding to the 3′ end of SEQ ID NO:137, but excluding the poly(A) tail at the 3′ end of SEQ ID NO:137. Also preferably the polynucleotide isolated according to the above process comprises a nucleotide sequence corresponding to the cDNA sequence of SEQ ID NO:137 from nucleotide 147 to nucleotide 1163, and extending contiguously from a nucleotide sequence corresponding to the 5′ end of said sequence of SEQ ID NO:137 from nucleotide 147 to nucleotide 1163, to a nucleotide sequence corresponding to the 3′ end of said sequence of SEQ ID NO:137 from nucleotide 147 to nucleotide 1163. Also preferably the polynucleotide isolated according to the above process comprises a nucleotide sequence corresponding to the cDNA sequence of SEQ ID NO:137 from nucleotide 273 to nucleotide 1163, and extending contiguously from a nucleotide sequence corresponding to the 5′ end of said sequence of SEQ ID NO:137 from nucleotide 273 to nucleotide 1163, to a nucleotide sequence corresponding to the 3′ end of said sequence of SEQ ID NO:137 from nucleotide 273 to nucleotide 1163.

In other embodiments, the present invention provides a composition comprising a protein, wherein said protein comprises an amino acid sequence selected from the group consisting of:

(a) the amino acid sequence of SEQ ID NO:138;

(b) fragments of the amino acid sequence of SEQ ID NO:138, each fragment comprising eight consecutive amino acids of SEQ ID NO:138; and

(c) the amino acid sequence encoded by the cDNA insert of clone mt24_{—}2 deposited with the ATCC under accession number 98636;

the protein being substantially free from other mammalian proteins. Preferably such protein comprises the amino acid sequence of SEQ ID NO:138. In further preferred embodiments, the present invention provides a protein comprising a fragment of the amino acid sequence of SEQ ID NO:138 having biological activity, the fragment preferably comprising eight (more preferably twenty, most preferably thirty) consecutive amino acids of SEQ ID NO:138, or a protein comprising a fragment of the amino acid sequence of SEQ ID NO:138 having biological activity, the fragment comprising the amino acid sequence from amino acid 164 to amino acid 173 of SEQ ID NO:138.

In one embodiment, the present invention provides a composition comprising an isolated polynucleotide selected from the group consisting of:

(a) a polynucleotide comprising the nucleotide sequence of SEQ ID NO:139;

(b) a polynucleotide comprising the nucleotide sequence of SEQ ID NO:139 from nucleotide 320 to nucleotide 1681;

(c) a polynucleotide comprising the nucleotide sequence of SEQ ID NO:139 from nucleotide 437 to nucleotide 1681;

(d) a polynucleotide comprising the nucleotide sequence of the full-length protein coding sequence of clone pe584_{—}2 deposited with the ATCC under accession number 98636;

(e) a polynucleotide encoding the full-length protein encoded by the cDNA insert of clone pe584_{—}2 deposited with the ATCC under accession number 98636;

(f) a polynucleotide comprising the nucleotide sequence of a mature protein coding sequence of clone pe584_{—}2 deposited with the ATCC under accession number 98636;

(g) a polynucleotide encoding a mature protein encoded by the cDNA insert of clone pe584_{—}2 deposited with the ATCC under accession number 98636;

(h) a polynucleotide encoding a protein comprising the amino acid sequence of SEQ ID NO:140;

(i) a polynucleotide encoding a protein comprising a fragment of the amino acid sequence of SEQ ID NO:140 having biological activity, the fragment comprising eight consecutive amino acids of SEQ ID NO:140;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:139 from nucleotide 320 to nucleotide 1681; the nucleotide sequence of SEQ ID NO:139 from nucleotide 437 to nucleotide 1681; the nucleotide sequence of the full-length protein coding sequence of clone pe584_{—}2 deposited with the ATCC under accession number 98636; or the nucleotide sequence of a mature protein coding sequence of clone pe584_{—}2 deposited with the ATCC under accession number 98636. In other preferred embodiments, the polynucleotide encodes the full-length or a mature protein encoded by the cDNA insert of clone pe584_{—}2 deposited with the ATCC under accession number 98636. In further preferred embodiments, the present invention provides a polynucleotide encoding a protein comprising a fragment of the amino acid sequence of SEQ ID NO:140 having biological activity, the fragment preferably comprising eight (more preferably twenty, most preferably thirty) consecutive amino acids of SEQ ID NO:140, or a polynucleotide encoding a protein comprising a fragment of the amino acid sequence of SEQ ID NO:140 having biological activity, the fragment comprising the amino acid sequence from amino acid 222 to amino acid 231 of SEQ ID NO:140.

Other embodiments provide the gene corresponding to the cDNA sequence of SEQ ID NO:139.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO: 139, but excluding the poly(A) tail at the 3′ end of SEQ ID NO:139; and

(ab) the nucleotide sequence of the cDNA insert of clone pe584_{—}2 deposited with the ATCC under accession number 98636;

(ii) hybridizing said probe(s) to human genornic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:139, but excluding the poly(A) tail at the 3′ end of SEQ ID NO:139; and

(bb) the nucleotide sequence of the cDNA insert of clone pe584_{—}2 deposited with the ATCC under accession number 98636;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:139, and extending contiguously from a nucleotide sequence corresponding to the 5′ end of SEQ ID NO:139 to a nucleotide sequence corresponding to the 3′ end of SEQ ID NO:139, but excluding the poly(A) tail at the 3′ end of SEQ ID NO:139. Also preferably the polynucleotide isolated according to the above process comprises a nucleotide sequence corresponding to the cDNA sequence of SEQ ID NO:139 from nucleotide 320 to nucleotide 1681, and extending contiguously from a nucleotide sequence corresponding to the 5′ end of said sequence of SEQ ID NO:139 from nucleotide 320 to nucleotide 1681, to a nucleotide sequence corresponding to the 3′ end of said sequence of SEQ ID NO:139 from nucleotide 320 to nucleotide 1681. Also preferably the polynucleotide isolated according to the above process comprises a nucleotide sequence corresponding to the cDNA sequence of SEQ ID NO:139 from nucleotide 437 to nucleotide 1681, and extending contiguously from a nucleotide sequence corresponding to the 5′ end of said sequence of SEQ ID NO:139 from nucleotide 437 to nucleotide 1681, to a nucleotide sequence corresponding to the 3′ end of said sequence of SEQ ID NO:139 from nucleotide 437 to nucleotide 1681.

In other embodiments, the present invention provides a composition comprising a protein, wherein said protein comprises an amino acid sequence selected from the group consisting of:

(a) the amino acid sequence of SEQ ID NO:140;

(b) fragments of the amino acid sequence of SEQ ID NO:140, each fragment comprising eight consecutive amino acids of SEQ ID NO:140; and

(c) the amino acid sequence encoded by the cDNA insert of clone pe584_{—}2 deposited with the ATCC under accession number 98636;

the protein being substantially free from other mammalian proteins. Preferably such protein comprises the amino acid sequence of SEQ ID NO:140. In further preferred embodiments, the present invention provides a protein comprising a fragment of the amino acid sequence of SEQ ID NO:140 having biological activity, the fragment preferably comprising eight (more preferably twenty, most preferably thirty) consecutive amino acids of SEQ ID NO:140, or a protein comprising a fragment of the amino acid sequence of SEQ ID NO:140 having biological activity, the fragment comprising the amino acid sequence from amino acid 222 to amino acid 231 of SEQ ID NO:140.

In one embodiment, the present invention provides a composition comprising an isolated polynucleotide selected from the group consisting of:

(a) a polynucleotide comprising the nucleotide sequence of SEQ ID NO:141;

(b) a polynucleotide comprising the nucleotide sequence of SEQ ID NO:141 from nucleotide 78 to nucleotide 1502;

(c) a polynucleotide comprising the nucleotide sequence of SEQ ID NO:141 from nucleotide 564 to nucleotide 1502;

(d) a polynucleotide comprising the nucleotide sequence of the full-length protein coding sequence of clone pj323_{—}2 deposited with the ATCC under accession number 98636;

(e) a polynucleotide encoding the full-length protein encoded by the cDNA insert of clone pj323_{—}2 deposited with the ATCC under accession number 98636;

(f) a polynucleotide comprising the nucleotide sequence of a mature protein coding sequence of clone pj323_{—}2 deposited with the ATCC under accession number 98636;

(g) a polynucleotide encoding a mature protein encoded by the cDNA insert of clone pj323_{—}2 deposited with the ATCC under accession number 98636;

(h) a polynucleotide encoding a protein comprising the amino acid sequence of SEQ ID NO:142;

(i) a polynucleotide encoding a protein comprising a fragment of the amino acid sequence of SEQ ID NO:142 having biological activity, the fragment comprising eight consecutive amino acids of SEQ ID NO:142;

(j) a polynucleotide which is an allelic variant of a polynucleotide of (a)-(g) above;

(k) a polynucleotide which encodes a species homologue of the protein of ) or (i) above; and

(l) a polynucleotide that hybridizes under stringent conditions to any one of the polynucleotides specified in (a)-(i).

Preferably, such polynucleotide comprises the nucleotide sequence of SEQ ID NO:141 fromnucleotide 78 to nucleotide 1502; the nucleotide sequence of SEQ ID NO:141 from nucleotide 564 to nucleotide 1502; the nucleotide sequence of the full-length protein coding sequence of clone pj323_{—}2 deposited with the ATCC under accession number 98636; or the nucleotide sequence of a mature protein coding sequence of clone pj323_{—}2 deposited with the ATCC under accession number 98636. In other preferred embodiments, the polynucleotide encodes the full-length or a mature protein encoded by the cDNA insert of clone pj323_{—}2 deposited with the ATCC under accession number 98636. In yet other preferred embodiments, the present invention provides a polynucleotide encoding a protein comprising the amino acid sequence of SEQ ID NO:142 from amino acid 54 to amino acid 145. In further preferred embodiments, the present invention provides a polynucleotide encoding a protein comprising a fragment of the amino acid sequence of SEQ ID NO:142 having biological activity, the fragment preferably comprising eight (more preferably twenty, most preferably thirty) consecutive amino acids of SEQ ID NO:142, or a polynucleotide encoding a protein comprising a fragment of the amino acid sequence of SEQ ID NO:142 having biological activity, the fragment comprising the amino acid sequence from amino acid 232 to amino acid 241 of SEQ ID NO: 142.

Other embodiments provide the gene corresponding to the cDNA sequence of SEQ ID NO:141.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:141, but excluding the poly(A) tail at the 3′ end of SEQ ID NO:141; and

(ab) the nucleotide sequence of the cDNA insert of clone pj323_{—}2 deposited with the ATCC under accession number 98636;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:141, but excluding the poly(A) tail at the 3′ end of SEQ ID NO:141; and

(bb) the nucleotide sequence of the cDNA insert of clone pj323_{—}2 deposited with the ATCC under accession number 98636;

(ii) hybridizing said primer(s) to human genonic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:141, and extending contiguously from a nucleotide sequence corresponding to the 5′ end of SEQ ID NO:141 to a nucleotide sequence corresponding to the 3′ end of SEQ ID NO:141, but excluding the poly(A) tail at the 3′ end of SEQ ID NO:141. Also preferably the polynucleotide isolated according to the above process comprises a nucleotide sequence corresponding to the cDNA sequence of SEQ ID NO:141 from nucleotide 78 to nucleotide 1502, and extending contiguously from a nucleotide sequence corresponding to the 5′ end of said sequence of SEQ ID NO:141 from nucleotide 78 to nucleotide 1502, to a nucleotide sequence corresponding to the 3′ end of said sequence of SEQ ID NO:141 from nucleotide 78 to nucleotide 1502. Also preferably the polynucleotide isolated according to the above process comprises a nucleotide sequence corresponding to the cDNA sequence of SEQ ID NO:141 from nucleotide 564 to nucleotide 1502, and extending contiguously from a nucleotide sequence corresponding to the 5′ end of said sequence of SEQ ID NO:141 from nucleotide 564 to nucleotide 1502, to a nucleotide sequence corresponding to the 3′ end of said sequence of SEQ ID NO:141 from nucleotide 564 to nucleotide 1502.

In other embodiments, the present invention provides a composition comprising a protein, wherein said protein comprises an amino acid sequence selected from the group consisting of:

(a) the amino acid sequence of SEQ ID NO:142;

(b) the amino acid sequence of SEQ ID NO:142 from amino acid 54 to amino acid 145;

(c) fragments of the amino acid sequence of SEQ ID NO:142, each fragment comprising eight consecutive amino acids of SEQ ID NO:142; and

(d) the amino acid sequence encoded by the cDNA insert of clone pj323_{—}2 deposited with the ATCC under accession number 98636;

the protein being substantially free from other mammalian proteins. Preferably such protein comprises the amino acid sequence of SEQ ID NO:142 or the amino acid sequence of SEQ ID NO:142 from amino acid 54 to amino acid 145. In further preferred embodiments, the present invention provides a protein comprising a fragment of the amino acid sequence of SEQ ID NO:142 having biological activity, the fragment preferably comprising eight (more preferably twenty, most preferably thirty) consecutive amino acids of SEQ ID NO:142, or a protein comprising a fragment of the amino acid sequence of SEQ ID NO:142 having biological activity, the fragment comprising the amino acid sequence from amino acid 232 to amino acid 241 of SEQ ID NO:142.

In one embodiment, the present invention provides a composition comprising an isolated polynucleotide selected from the group consisting of:

(a) a polynucleotide comprising the nucleotide sequence of SEQ ID NO:143;

(b) a polynucleotide comprising the nucleotide sequence of SEQ ID NO:143 from nucleotide 130 to nucleotide 294;

(c) a polynucleotide comprising the nucleotide sequence of SEQ ID NO:143 from nucleotide 241 to nucleotide 294;

(d) a polynucleotide comprising the nucleotide sequence of the full-length protein coding sequence of clone yb24_{—}1 deposited with the ATCC under accession number 98636;

(e) a polynucleotide encoding the full-length protein encoded by the cDNA insert of clone yb24_{—}1 deposited with the ATCC under accession number 98636;

(f) a polynucleotide comprising the nucleotide sequence of a mature protein coding sequence of clone yb24_{—}1 deposited with the ATCC under accession numnber 98636;

(g) a polynucleotide encoding a mature protein encoded by the cDNA insert of clone yb24_{—}1 deposited with the ATCC under accession number 98636;

(h) a polynucleotide encoding a protein comprising the amino acid sequence of SEQ ID NO:144;

(i) a polynucleotide encoding a protein comprising a fragment of the amino acid sequence of SEQ ID NO:144 having biological activity, the fragment comprising eight consecutive amino acids of SEQ ID NO:144;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:143 from nucleotide 130 to nucleotide 294; the nucleotide sequence of SEQ ID NO:143 from nucleotide 241 to nucleotide 294; the nucleotide sequence of the full-length protein coding sequence of clone yb24_{—}1 deposited with the ATCC under accession number 98636; or the nucleotide sequence of a mature protein coding sequence of clone yb24_{—}1 deposited with the ATCC under accession number 98636. In other preferred embodiments, the polynucleotide encodes the full-length or a mature protein encoded by the cDNA insert of clone yb24_{—}1 deposited with the ATCC under accession number 98636. In further preferred embodiments, the present invention provides a polynucleotide encoding a protein comprising a fragment of the amino acid sequence of SEQ ID NO:144 having biological activity, the fragment preferably comprising eight (more preferably twenty, most preferably thirty) consecutive amino acids of SEQ ID NO:144, or a polynucleotide encoding a protein comprising a fragment of the amino acid sequence of SEQ ID NO:144 having biological activity, the fragment comprising the amino acid sequence from amino acid 22 to amino acid 31 of SEQ ID NO:144.

Other embodiments provide the gene corresponding to the cDNA sequence of SEQ ID NO:143.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:143, but excluding the poly(A) tail at the 3′ end of SEQ ID NO:143; and

(ab) the nucleotide sequence of the cDNA insert of clone yb24_{—}1 deposited with the ATCC under accession number 98636;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:143, but excluding the poly(A) tail at the 3′ end of SEQ ID NO:143; and

(bb) the nucleotide sequence of the cDNA insert of clone yb24_{—}1 deposited with the ATCC under accession number 98636;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a -nucleotide sequence corresponding to the cDNA sequence of SEQ ID NO:143, and extending contiguously from a nucleotide sequence corresponding to the 5′ end of SEQ ID NO:143 to a nucleotide sequence corresponding to the 3′ end of SEQ ID NO:143, but excluding the poly(A) tail at the 3′ end of SEQ ID NO:143. Also preferably the polynucleotide isolated according to the above process comprises a nucleotide sequence corresponding to the cDNA sequence of SEQ ID NO:143 from nucleotide 130 to nucleotide 294, and extending contiguously from a nucleotide sequence corresponding to the 5′ end of said sequence of SEQ ID NO:143 from nucleotide 130 to nucleotide 294, to a nucleotide sequence corresponding to the 3′ end of said sequence of SEQ ID NO:143 from nucleotide 130 to nucleotide 294. Also preferably the polynucleotide isolated according to the above process comprises a nucleotide sequence corresponding to the cDNA sequence of SEQ ID NO:143 from nucleotide 241 to nucleotide 294, and extending contiguously from a nucleotide sequence corresponding to the 5′ end of said sequence of SEQ ID NO:143 from nucleotide 241 to nucleotide 294, to a nucleotide sequence corresponding to the 3′ end of said sequence of SEQ ID NO:143 from nucleotide 241 to nucleotide 294.

In other embodiments, the present invention provides a composition comprising a protein, wherein said protein comprises an amino acid sequence selected from the group consisting of:

(a) the amino acid sequence of SEQ ID NO:144;

(b) fragments of the amino acid sequence of SEQ ID NO:144, each fragment comprising eight consecutive amino acids of SEQ ID NO:144; and

(c) the amino acid sequence encoded by the cDNA insert of clone yb24_{—}1 deposited with the ATCC under accession number 98636;

the protein being substantially free from other mammalian proteins. Preferably such protein comprises the amino acid sequence of SEQ ID NO:144. In further preferred embodiments, the present invention provides a protein comprising a fragment of the amino acid sequence of SEQ ID NO:144 having biological activity, the fragment preferably comprising eight (more preferably twenty, most preferably thirty) consecutive amino acids of SEQ ID NO:144, or a protein comprising a fragment of the amino acid sequence of SEQ ID NO:144 having biological activity, the fragment comprising the amino acid sequence from amino acid 22 to amino acid 31 of SEQ ID NO:144.

In one embodiment, the present invention provides a composition comprising an isolated polynucleotide selected from the group consisting of:

(a) a polynucleotide comprising the nucleotide sequence of SEQ ID NO:145;

(b) a polynucleotide comprising the nucleotide sequence of SEQ ID NO:145 from nucleotide 514 to nucleotide 1707;

(c) a polynucleotide comprising the nucleotide sequence of SEQ ID NO:145 from nucleotide 580 to nucleotide 1707;

(d) a polynucleotide comprising the nucleotide sequence of the full-length protein coding sequence of clone yb44_{—}1 deposited with the ATCC under accession number 98636;

(e) a polynucleotide encoding the full-length protein encoded by the cDNA insert of clone yb44_{—}1 deposited with the ATCC under accession number 98636;

(f) a polynucleotide comprising the nucleotide sequence of a mature protein coding sequence of clone yb44_{—}1 deposited with the ATCC under accession number 98636;

(g) a polynucleotide encoding a mature protein encoded by the cDNA insert of clone yb44_{—}1 deposited with the ATCC under accession number 98636;

(h) a polynucleotide encoding a protein comprising the amino acid sequence of SEQ ID NO:146;

(i) a polynucleotide encoding a protein comprising a fragment of the amino acid sequence of SEQ ID NO:146 having biological activity, the fragment comprising eight consecutive amino acids of SEQ ID NO:146;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:145 from nucleotide 514 to nucleotide 1707; the nucleotide sequence of SEQ ID NO:145 from nucleotide 580 to nucleotide 1707; the nucleotide sequence of the full-length protein coding sequence of clone yb44_{—}1 deposited with the ATCC under accession number 98636; or the nucleotide sequence of a mature protein coding sequence of clone yb44_{—}1 deposited with the ATCC under accession number 98636. In other preferred embodiments, the polynucleotide encodes the full-length or a mature protein encoded by the cDNA insert of clone yb44_{—}1 deposited with the ATCC under accession number 98636. In further preferred embodiments, the present invention provides a polynucleotide encoding a protein comprising a fragment of the amino acid sequence of SEQ ID NO:146 having biological activity, the fragment preferably comprising eight (more preferably twenty, most preferably thirty) consecutive amino acids of SEQ ID NO:146, or a polynucleotide encoding a protein comprising a fragment of the amnino acid sequence of SEQ ID NO:146 having biological activity, the fragment comprising the amino acid sequence from amino acid 194 to amino acid 203 of SEQ ID NO:146.

Other embodiments provide the gene corresponding to the cDNA sequence of SEQ ID NO:145.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQIDNO:145,butexcludingthepoly(A) tail atthe 3′ end of SEQ ID NO:145; and

(ab) the nucleotide sequence of the cDNA insert of clone yb44_{—}1 deposited with the ATCC under accession number 98636;

(ii) hybridizing said probe(s) to human genomnic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:145, but excluding the poly(A) tail at the 3′ end of SEQ ID NO:145; and

(bb) the nucleotide sequence of the cDNA insert of clone yb44_{—}1 deposited with the ATCC under accession number 98636;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:145, and extending contiguously from a nucleotide sequence corresponding to the 5′ end of SEQ ID NO:145 to a nucleotide sequence corresponding to the 3′ end of SEQ ID NO:145, but excluding the poly(A) tail at the 3′ end of SEQ ID NO:145. Also preferably the polynucleotide isolated according to the above process comprises a nucleotide sequence corresponding to the cDNA sequence of SEQ ID NO:145 from nucleotide 514 to nucleotide 1707, and extending contiguously from a nucleotide sequence corresponding to the 5′ end of said sequence of SEQ ID NO:145 from nucleotide 514 to nucleotide 1707, to a nucleotide sequence corresponding to the 3′ end of said sequence of SEQ ID NO:145 from nucleotide 514 to nucleotide 1707. Also preferably the polynucleotide isolated according to the above process comprises a nucleotide sequence corresponding to the cDNA sequence of SEQ ID NO:145 from nucleotide 580 to nucleotide 1707, and extending contiguously from a nucleotide sequence corresponding to the 5′ end of said sequence of SEQ ID NO:145 from nucleotide 580 to nucleotide 1707, to a nucleotide sequence corresponding to the 3′ end of said sequence of SEQ ID NO:145 from nucleotide 580 to nucleotide 1707.

In other embodiments, the present invention provides a composition comprising a protein, wherein said protein comprises an amino acid sequence selected from the group consisting of:

(a) the amino acid sequence of SEQ ID NO:146;

(b) fragments of the amino acid sequence of SEQ ID NO:146, each fragment comprising eight consecutive amino acids of SEQ ID NO:146; and

(c) the amino acid sequence encoded by the cDNA insert of clone yb44_{—}1 deposited with the ATCC under accession number 98636;

the protein being substantially free from other mammalian proteins. Preferably such protein comprises the amino acid sequence of SEQ ID NO:146. In further preferred embodiments, the present invention provides a protein comprising a fragment of the amino acid sequence of SEQ ID NO:146 having biological activity, the fragment preferably comprising eight (more preferably twenty, most preferably thirty) consecutive amino acids of SEQ ID NO:146, or a protein comprising a fragment of the amino acid sequence of SEQ ID NO:146 having biological activity, the fragment comprising the amino acid sequence from amino acid 194 to amino acid 203 of SEQ ID NO: 146.

In one embodiment, the present invention provides a composition comprising an isolated polynucleotide selected from the group consisting of:

(a) a polynucleotide comprising the nucleotide sequence of SEQ ID NO:147;

(b) a polynucleotide comprising the nucleotide sequence of SEQ ID NO:147 from nucleotide 1529 to nucleotide 1726;

(c) a polynucleotide comprising the nucleotide sequence of SEQ ID NO:147 from nucleotide 1706 to nucleotide 1726;

(d) a polynucleotide comprising the nucleotide sequence of the full-length protein coding sequence of clone bn69_{—}15 deposited with the ATCC under accession number 98647;

(e) a polynucleotide encoding the full-length protein encoded by the cDNA insert of clone bn69_{—}15 deposited with the ATCC under accession number 98647;

(f) a polynucleotide comprising the nucleotide sequence of a mature protein coding sequence of clone bn69_{—}15 deposited with the ATCC under accession number 98647;

(g) a polynucleotide encoding a mature protein encoded by the cDNA insert of clone bn69_{—}15 deposited with the ATCC under accession number 98647;

(h) a polynucleotide encoding a protein comprising the amino acid sequence of SEQ ID NO:148;

(i) a polynucleotide encoding a protein comprising a fragment of the amino acid sequence of SEQ ID NO: 148 having biological activity, the fragment comprising eight consecutive amino acids of SEQ ID NO:148;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:147 from nucleotide 1529 to nucleotide 1726; the nucleotide sequence of SEQ ID NO:147 from nucleotide 1706 to nucleotide 1726; the nucleotide sequence of the full-length protein coding sequence of clone bn69_{—}15 deposited with the ATCC under accession number 98647; or the nucleotide sequence of a mature protein coding sequence of clone bn69_{—}15 deposited with the ATCC under accession number 98647. In other preferred embodiments, the polynucleotide encodes the full-length or a mature protein encoded by the cDNA insert of clone bn69_{—}15 deposited with the ATCC under accession number 98647. In yet other preferred embodiments, the present invention provides a polynucleotide encoding a protein comprising the amino acid sequence of SEQ ID NO:148 from amino acid 1 to amino acid 53. In further preferred embodiments, the present invention provides a polynucleotide encoding a protein comprising a fragment of the amino acid sequence of SEQ ID NO:148 having biological activity, the fragment preferably comprising eight (more preferably twenty, most preferably thirty) consecutive amino acids of SEQ ID NO:148, or a polynucleotide encoding a protein comprising a fragment of the amino acid sequence of SEQ ID NO:148 having biological activity, the fragment comprising the amino acid sequence from amino acid 28 to amino acid 37 of SEQ ID NO:148.

Other embodiments provide the gene corresponding to the cDNA sequence of SEQ ID NO:147.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:147,but excluding the poly(A) tail at the 3′ end of SEQ ID NO:147; and

(ab) the nucleotide sequence of the cDNA insert of clone bn69_{—}15 deposited with the ATCC under accession number 98647;

(ii) hybridizing said probe(s) to human genomnic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:147, but excluding the poly(A) tail at the 3′ end of SEQ ID NO:147; and

(bb) the nucleotide sequence of the cDNA insert of clone bn69_{—}15 deposited with the ATCC under accession number 98647;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:147, and extending contiguously from a nucleotide sequence corresponding to the 5′ end of SEQ ID NO:147 to a nucleotide sequence corresponding to the 3′ end of SEQ ID NO:147, but excluding the poly(A) tail at the 3′ end of SEQ ID NO:147. Also preferably the polynucleotide isolated according to the above process comprises a nucleotide sequence corresponding to the cDNA sequence of SEQ ID NO:147 from nucleotide 1529 to nucleotide 1726, and extending contiguously from a nucleotide sequence corresponding to the 5′ end of said sequence of SEQ ID NO:147 from nucleotide 1529 to nucleotide 1726, to a nucleotide sequence corresponding to the 3′ end of said sequence of SEQ ID NO:147 from nucleotide 1529 to nucleotide 1726. Also preferably the polynucleotide isolated according to the above process comprises a nucleotide sequence corresponding to the cDNA sequence of SEQ ID NO:147 from nucleotide 1706 to nucleotide 1726, and extending contiguously from a nucleotide sequence corresponding to the 5′ end of said sequence of SEQ ID NO:147 from nucleotide 1706 to nucleotide 1726, to a nucleotide sequence corresponding to the 3′ end of said sequence of SEQ ID NO: 147 from nucleotide 1706 to nucleotide 1726.

In other embodiments, the present invention provides a composition comprising a protein, wherein said protein comprises an amino acid sequence selected from the group consisting of:

(a) the amino acid sequence of SEQ ID NO:148;

(b) the amino acid sequence of SEQ ID NO:148 from amino acid 1 to amino acid 53;

(c) fragments of the amino acid sequence of SEQ ID NO:148, each fragment comprising eight consecutive amino acids of SEQ ID NO:148; and

(d) the amino acid sequence encoded by the cDNA insert of clone bn69_{—}15 deposited with the ATCC under accession number 98647;

the protein being substantially free from other mammalian proteins. Preferably such protein comprises the amino acid sequence of SEQ ID NO:148 or the amino acid sequence of SEQ ID NO:148 from amino acid 1 to amino acid 53. In further preferred embodiments, the present invention provides a protein comprising a fragment of the amino acid sequence of SEQ ID NO:148 having biological activity, the fragment preferably comprising eight (more preferably twenty, most preferably thirty) consecutive amino acids of SEQ ID NO:148, or a protein comprising a fragment of the amino acid sequence of SEQ ID NO:148 having biological activity, the fragment comprising the amino acid sequence from amino acid 28 to amino acid 37 of SEQ ID NO:148.

In one embodiment, the present invention provides a composition comprising an isolated polynucleotide selected from the group consisting of:

(a) a polynucleotide comprising the nucleotide sequence of SEQ ID NO:149;

(b) a polynucleotide comprising the nucleotide sequence of SEQ ID NO: 149 from nucleotide 334 to nucleotide 597;

(c) a polynucleotide comprising the nucleotide sequence of SEQ ID NO:149 from nucleotide 478 to nucleotide 597;

(d) a polynucleotide comprising the nucleotide sequence of the full-length protein coding sequence of clone cb110_{—}1 deposited with the ATCC under accession number 98647;

(e) a polynucleotide encoding the full-length protein encoded by the cDNA insert of clone cb110_{—}1 deposited with the ATCC under accession number 98647;

(f) a polynucleotide comprising the nucleotide sequence of a mature protein coding sequence of clone cb110_{—}1 deposited with the ATCC under accession number 98647;

(g) a polynucleotide encoding a mature protein encoded by the cDNA insert of clone cb110_{—}1 deposited with the ATCC under accession number 98647;

(h) a polynucleotide encoding a protein comprising the amino acid sequence of SEQ ID NO:150;

(i) a polynucleotide encoding a protein comprising a fragment of the amino acid sequence of SEQ ID NO:150 having biological activity, the fragment comprising eight consecutive amino acids of SEQ ID NO:150;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:149 from nucleotide 334 to nucleotide 597; the nucleotide sequence of SEQ ID NO:149 from nucleotide 478 to nucleotide 597; the nucleotide sequence of the full-length protein coding sequence of clone cb110_{—}1 deposited with the ATCC under accession number 98647; or the nucleotide sequence of a mature protein coding sequence of clone cbllO_{—}1 deposited with the ATCC under accession number 98647. In other preferred embodiments, the polynucleotide encodes the full-length or a mature protein encoded by the cDNA insert of clone cb110_{—}1 deposited with the ATCC under accession number 98647. In further preferred embodiments, the present invention provides a polynucleotide encoding a protein comprising a fragment of the amino acid sequence of SEQ ID NO:150 having biological activity, the fragment preferably comprising eight (more preferably twenty, most preferably thirty) consecutive amino acids of SEQ ID NO:150, or a polynucleotide encoding a protein comprising a fragment of the amino acid sequence of SEQ ID NO:150 having biological activity, the fragment comprising the amino acid sequence from amino acid 39 to amino acid 48 of SEQ ID NO:150.

Other embodiments provide the gene corresponding to the cDNA sequence of SEQ ID NO:149.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:149, but excluding the poly(A) tail at the 3′ end of SEQ ID NO:149; and

(ab) the nucleotide sequence of the cDNA insert of clone cb110_{—}1 deposited with the ATCC under accession number 98647;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO: 149, but excluding the poly(A) tail at the 3′ end of SEQ ID NO:149; and

(bb) the nucleotide sequence of the cDNA insert of clone cb110_{—}1 deposited with the ATCC under accession number 98647;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:149, and extending contiguously from a nucleotide sequence corresponding to the 5′ end of SEQ ID NO:149 to a nucleotide sequence corresponding to the 3′ end of SEQ ID NO:149, but excluding the poly(A) tail at the 3′ end of SEQ ID NO:149. Also preferably the polynucleotide isolated according to the above process comprises a nucleotide sequence corresponding to the cDNA sequence of SEQ ID NO:149 from nucleotide 334 to nucleotide 597, and extending contiguously from a nucleotide sequence corresponding to the 5′ end of said sequence of SEQ ID NO:149 from nucleotide 334 to nucleotide 597, to a nucleotide sequence corresponding to the 3′ end of said sequence of SEQ ID NO:149 from nucleotide 334 to nucleotide 597. Also preferably the polynucleotide isolated according to the above process comprises a nucleotide sequence corresponding to the cDNA sequence of SEQ ID NO: 149 from nucleotide 478 to nucleotide 597, and extending contiguously from a nucleotide sequence corresponding to the 5′ end of said sequence of SEQ ID NO:149 from nucleotide 478 to nucleotide 597, to a nucleotide sequence corresponding to the 3′ end of said sequence of SEQ ID NO:149 from nucleotide 478 to nucleotide 597.

In other embodiments, the present invention provides a composition comprising a protein, wherein said protein comprises an amino acid sequence selected from the group consisting of:

(a) the amino acid sequence of SEQ ID NO:150;

(b) fragments of the amino acid sequence of SEQ ID NO:150, each fragment comprising eight consecutive amino acids of SEQ ID NO:150; and

(c) the amino acid sequence encoded by the cDNA insert of clone cb110_{—}1 deposited with the ATCC under accession number 98647;

the protein being substantially free from other mammalian proteins. Preferably such protein comprises the amino acid sequence of SEQ ID NO:150. In further preferred embodiments, the present invention provides a protein comprising a fragment of the amino acid sequence of SEQ ID NO:150 having biological activity, the fragment preferably comprising eight (more preferably twenty, most preferably thirty) consecutive amino acids of SEQ ID NO:150, or a protein comprising a fragment of the amino acid sequence of SEQ ID NO:150 having biological activity, the fragment comprising the amino acid sequence from amino acid 39 to amino acid 48 of SEQ ID NO:150.

In one embodiment, the present invention provides a composition comprising an isolated polynucleotide selected from the group consisting of:

(a) a polynucleotide comprising the nucleotide sequence of SEQ ID NO:151;

(b) a polynucleotide comprising the nucleotide sequence of SEQ ID NO: 151 from nucleotide 191 to nucleotide 1132;

(c) a polynucleotide comprising the nucleotide sequence of SEQ ID NO:151 from nucleotide 290 to nucleotide 1132;

(d) a polynucleotide comprising the nucleotide sequence of the full-length protein coding sequence of clone ch4_{—}11 deposited with the ATCC under accession number 98647;

(e) a polynucleotide encoding the full-length protein encoded by the cDNA insert of clone ch4_{—}11 deposited with the ATCC under accession number 98647;

(f) a polynucleotide comprising the nucleotide sequence of a mature protein coding sequence of clone ch4_{—}11 deposited with the ATCC under accession number 98647;

(g) a polynucleotide encoding a mature protein encoded by the cDNA insert of clone ch4_{—}11 deposited with the ATCC under accession number 98647;

(h) a polynucleotide encoding a protein comprising the amino acid sequence of SEQ ID NO:152;

(i) a polynucleotide encoding a protein comprising a fragment of the amino acid sequence of SEQ ID NO: 152 having biological activity, the fragment comprising eight consecutive amino acids of SEQ ID NO: 152;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:151 from nucleotide 191 to nucleotide 1132; the nucleotide sequence of SEQ ID NO:151 from nucleotide 290 to nucleotide 1132; the nucleotide sequenceof the full-length protein coding sequence of clone ch4_{—}11 deposited with the ATCC under accession number 98647; or the nucleotide sequence of a mature protein coding sequence of clone ch4_{—}11 deposited with the ATCC under accession number 98647. In other preferred embodiments, the polynucleotide encodes the full-length or a mature protein encoded by the cDNA insert of clone ch4_{—}11 deposited with the ATCC under accession number 98647.

In further preferred embodiments, the present invention provides a polynucleotide encoding a protein comprising a fragment of the amino acid sequence of SEQ ID NO:152 having biological activity, the fragment preferably comprising eight (more preferably twenty, most preferably thirty) consecutive amino acids of SEQ ID NO:152, or a polynucleotide encoding a protein comprising a fragment of the amino acid sequence of SEQ ID NO:152 having biological activity, the fragment comprising the amino acid sequence from amino acid 152 to amino acid 161 of SEQ ID NO:152.

Other embodiments provide the gene corresponding to the cDNA sequence of SEQ ID NO:151.

Further embodiments of the invention provide isolated polynucleotides produced according to a process selected from the group consisting of:

(a) a process comprising the steps of:

(i) preparing one or more polynucleotideprobes thathybridize in 6×SSC at 65 degrees C to a nucleotide sequence selected from the group consisting of:

(aa) SEQ IDNO:151, but excluding the poly(A) tail at the 3′ end of SEQ ID NO:151; and

(ab) the nucleo tide sequence of the cDNA insert of clone ch4_{—}11 deposited with the ATCC under accession number 98647;

(ii) hybridizing said probe(s) to human genomic DINA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:151,butexcludingthepoly(A) tail atthe 3′ end of SEQ ID NO:151; and

(bb) the nucleotide sequence of the cDNA insert of clone ch4_{—}11 deposited with the ATCC under accession number 98647;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:151, and extending contiguously from a nucleotide sequence corresponding to the 5′ end of SEQ ID NO:151 to a nucleotide sequence corresponding to the 3′ end of SEQ ID NO:151, but excluding the poly(A) tail at the 3′ end of SEQ ID NO:151. Also preferably the polynucleotide isolated according to the above process comprises a nucleotide sequence corresponding to the cDNA sequence of SEQ ID NO:151 from nucleotide 191 to nucleotide 1132, and extending contiguously from a nucleotide sequence corresponding to the 5′ end of said sequence of SEQ ID NO:151 from nucleotide 191 to nucleotide 1132, to a nucleotide sequence corresponding to the 3′ end of said sequence of SEQ ID NO:151 from nucleotide 191 to nucleotide 1132. Also preferably the polynucleotide isolated according to the above process comprises a nucleotide sequence corresponding to the cDNA sequence of SEQ ID NO:151 from nucleotide 290 to nucleotide 1132, and extending contiguously from a nucleotide sequence corresponding to the 5′ end of said sequence of SEQ ID NO:151 from nucleotide 290 to nucleotide 1132, to a nucleotide sequence corresponding to the 3′ end of said sequence of SEQ ID NO:151 from nucleotide 290 to nucleotide 1132.

In other embodiments, the present invention provides a composition comprising a protein, wherein said protein comprises an amino acid sequence selected from the group consisting of:

(a) the amino acid sequence of SEQ ID NO:152;

(b) fragments of the amino acid sequence of SEQ ID NO:152, each fragment comprising eight consecutive amino acids of SEQ ID NO:152; and

(c) the amino acid sequence encoded by the cDNA insert of clone ch4_{—}11 deposited with the ATCC under accession number 98647;

the protein being substantially free from other mammalian proteins. Preferably such protein comprises the amino acid sequence of SEQ ID NO:152. In further preferred embodiments, the present invention provides a protein comprising a fragment of the amino acid sequence of SEQ ID NO:152 having biological activity, the fragment preferably comprising eight (more preferably twenty, most preferably thirty) consecutive amino acids of SEQ ID NO:152, or a protein comprising a fragment of the amino acid sequence of SEQ ID NO:152 having biological activity, the fragment comprising the amino acid sequence from amino acid 152 to amino acid 161 of SEQ ID NO:152.

In one embodiment, the present invention provides a composition comprising an isolated polynucleotide selected from the group consisting of:

(a) a polynucleotide comprising the nucleotide sequence of SEQ ID NO:153;

(b) a polynucleotide comprising the nucleotide sequence of SEQ ID NO:153 from nucleotide 732 to nucleotide 1898;

(c) a polynucleotide comprising the nucleotide sequence of the full-length protein coding sequence of clone cn621_{—}8 deposited with the ATCC under accession number 98647;

(d) a polynucleotide encoding the full-length protein encoded by the cDNA insert of clone cn621_{—}8 deposited with the ATCC under accession number 98647;

(e) a polynucleotide comprising the nucleotide sequence of a mature protein coding sequence of clone cn621_{—}8 deposited with the ATCC under accession number 98647;

(f) a polynucleotide encoding a mature protein encoded by the cDNA insert of clone cn621_{—}8 deposited with the ATCC under accession number 98647;

(g) a polynucleotide encoding a protein comprising the amino acid sequence of SEQ ID NO:154;

(h) a polynucleotide encoding a protein comprising a fragment of the amino acid sequence of SEQ ID NO:154 having biological activity, the fragment comprising eight consecutive amino acids of SEQ ID NO:154;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:153 from nucleotide 732 to nucleotide 1898; the nucleotide sequence of the full-length protein coding sequence of clone cn621_{—}8 deposited with the ATCC under accession number 98647; or the nucleotide sequence of a mature protein coding sequence of clone cn621_{—}8 deposited with the ATCC under accession number 98647. In other preferred embodiments, the polynucleotide encodes the full-length or a mature protein encoded by the cDNA insert of clone cn621_{—}8 deposited with the ATCC under accession number 98647. In further preferred embodiments, the present invention provides a polynucleotide encoding a protein comprising a fragment of the amino acid sequence of SEQ ID NO:154 having biological activity, the fragment preferably comprising eight (more preferably twenty, most preferably thirty) consecutive amino acids of SEQ ID NO:154, or a polynucleotide encoding a protein comprising a fragment of the amino acid sequence of SEQ ID NO:154 having biological activity, the fragment comprising the amino acid sequence from amino acid 189 to amino acid 198 of SEQ ID NO:154.

Other embodiments provide the gene corresponding to the cDNA sequence of SEQ ID NO:153.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:153, but excluding the poly(A) tail at the 3′ end of SEQ ID NO:153; and

(ab) the nucleotide sequence of the cDNA insert of clone cn621_{—}8 deposited with the ATCC under accession number 98647;

(ii) hybridizing said probe(s) to human genornic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO: 153, but excluding the poly(A) tail at the 3′ end of SEQ ID NO:153; and

(bb) the nucleotide sequence of the cDNA insert of clone cn621_{—}8 deposited with the ATCC under accession number 98647;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:153, and extending contiguously from a nucleotide sequence corresponding to the 5′ end of SEQ ID NO:153 to a nucleotide sequence corresponding to the 3′ end of SEQ ID NO:153, but excluding the poly(A) tail at the 3′ end of SEQ ID NO:153. Also preferably the polynucleotide isolated according to the above process comprises a nucleotide sequence corresponding to the cDNA sequence of SEQ ID NO:153 from nucleotide 732 to nucleotide 1898, and extending contiguously from a nucleotide sequence corresponding to the 5′ end of said sequence of SEQ ID NO:153 from nucleotide 732 to nucleotide 1898, to a nucleotide sequence corresponding to the 3′ end of said sequence of SEQ ID NO:153 from nucleotide 732 to nucleotide 1898.

In other embodiments, the present invention provides a composition comprising a protein, wherein said protein comprises an amino acid sequence selected from the group consisting of:

(a) the amino acid sequence of SEQ ID NO:154;

(b) fragments of the amino acid sequence of SEQ ID NO:154, each fragment comprising eight consecutive amino acids of SEQ ID NO:154; and

(c) the amino acid sequence encoded by the cDNA insert of clone cn621_{—}8 deposited with the ATCC under accession number 98647;

the protein being substantially free from other mammalian proteins. Preferably such protein comprises the amino acid sequence of SEQ ID NO:154. In further preferred embodiments, the present invention provides a protein comprising a fragment of the amino acid sequence of SEQ ID NO:154 having biological activity, the fragment preferably comprising eight (more preferably twenty, most preferably thirty) consecutive amino acids of SEQ ID NO:154, or a protein comprising a fragment of the amino acid sequence of SEQ ID NO:154 having biological activity, the fragment comprising the amino acid sequence from amino acid 189 to amino acid 198 of SEQ ID NO:154.

In one embodiment, the present invention provides a composition comprising an isolated polynucleotide selected from the group consisting of:

(a) a polynucleotide comprising the nucleotide sequence of SEQ ID NO:155;

(b) a polynucleotide comprising the nucleotide sequence of SEQ ID NO:155 from nucleotide 308 to nucleotide 592;

(c) a polynucleotide comprising the nucleotide sequence of SEQ ID NO:155 from nucleotide 377 to nucleotide 592;

(d) a polynucleotide comprising the nucleotide sequence of the full-length protein coding sequence of clone gy621_{—}1 deposited with the ATCC under accession number 98647;

(e) a polynucleotide encoding the full-length protein encoded by the cDNA insert of clone gy621_{—}1 deposited with the ATCC under accession number 98647;

(f) a polynucleotide comprising the nucleotide sequence of a mature protein coding sequence of clone gy621_{—}1 deposited with the ATCC under accession number 98647;

(g) a polynucleotide encoding a mature protein encoded by the cDNA insert of clone gy621_{—}1 deposited with the ATCC under accession number 98647;

(h) a polynucleotide encoding a protein comprising the amnino acid sequence of SEQ ID NO:156;

(i) a polynucleotide encoding a protein comprising a fragment of the amino acid sequence of SEQ ID NO:156 having biological activity, the fragment comprising eight consecutive amino acids of SEQ ID NO: 156;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:155 fromnucleotide 308 to nucleotide 592; the nucleotide sequence of SEQ ID NO:155 from nucleotide 377 to nucleotide 592; the nucleotide sequence of the full-length protein coding sequence of clone gy621_{—}1 deposited with the ATCC under accession number 98647; or the nucleotide sequence of a mature protein coding sequence of clone gy621_{—}1 deposited with the ATCC under accession number 98647. In other preferred embodiments, the polynucleotide encodes the full-length or a mature protein encoded by the cDNA insert of clone gy621_{—}1 deposited with the ATCC under accession number 98647. In further preferred embodiments, the present invention provides a polynucleotide encoding a protein comprising a fragment of the amino acid sequence of SEQ ID NO:156 having biological activity, the fragment preferably comprising eight (more preferably twenty, most preferably thirty) consecutive amino acids of SEQ ID NO:156, or a polynucleotide encoding a protein comprising a fragment of the amino acid sequence of SEQ ID NO:156 having biological activity, the fragment comprising the amino acid sequence from amino acid 42 to amino acid 51 of SEQ ID NO:156.

Other embodiments provide the gene corresponding to the cDNA sequence of SEQ ID NO:155.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO: 155, but excluding the poly(A) tail at the 3′ end of SEQ ID NO:155; and

(ab) the nucleotide sequence of the cDNA insert of clone gy621_{—}1 deposited with the ATCC under accession number 98647;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:155, but excluding the poly(A) tail at the 3′ end of SEQ ID NO:155; and

(bb) the nucleotide sequence of the cDNA insert of clone gy621_{—}1 deposited with the ATCC under accession number 98647;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:155, and extending contiguously from a nucleotide sequence corresponding to the 5′ end of SEQ ID NO:155 to a nucleotide sequence corresponding to the 3′ end of SEQ ID NO:155, but excluding the poly(A) tail at the 3′ end of SEQ ID NO:155. Also preferably the polynucleotide isolated according to the above process comprises a nucleotide sequence corresponding to the cDNA sequence of SEQ ID NO:155 from nucleotide 308 to nucleotide 592, and extending contiguously from a nucleotide sequence corresponding to the 5′ end of said sequence of SEQ ID NO:155 from nucleotide 308 to nucleotide 592, to a nucleotide sequence corresponding to the 3′ end of said sequence of SEQ ID NO:155 from nucleotide 308 to nucleotide 592. Also preferably the polynucleotide isolated according to the above process comprises a nucleotide sequence corresponding to the cDNA sequence of SEQ ID NO:155 from nucleotide 377 to nucleotide 592, and extending contiguously from a nucleotide sequence corresponding to the 5′ end of said sequence of SEQ ID NO:155 from nucleotide 377 to nucleotide 592, to a nucleotide sequence corresponding to the 3′ end of said sequence of SEQ ID NO:155 from nucleotide 377 to nucleotide 592.

In other embodiments, the present invention provides a composition comprising a protein, wherein said protein comprises an amino acid sequence selected from the group consisting of:

(a) the amino acid sequence of SEQ ID NO:156;

(b) fragments of the amino acid sequence of SEQ ID NO:156, each fragment comprising eight consecutive amino acids of SEQ ID NO:156; and

(c) the amino acid sequence encoded by the cDNA insert of clone gy621_{—}1 deposited with the ATCC under accession number 98647;

the protein being substantially free from other mammalian proteins. Preferably such protein comprises the amino acid sequence of SEQ ID NO:156. In further preferred embodiments, the present invention provides a protein comprising a fragment of the amino acid sequence of SEQ ID NO:156 having biological activity, the fragment preferably comprising eight (more preferably twenty, most preferably thirty) consecutive amino acids of SEQ ID NO:156, or a protein comprising a fragment of the amino acid sequence of SEQ ID NO:156 having biological activity, the fragment comprising the amino acid sequence from amino acid 42 to amino acid 51 of SEQ ID NO:156.

In one embodiment, the present invention provides a composition comprising an isolated polynucleotide selected from the group consisting of:

(a) a polynucleotide comprising the nucleotide sequence of SEQ ID NO:157;

(b) a polynucleotide comprising the nucleotide sequence of SEQ ID NO:157 from nucleotide 124 to nucleotide 1881;

(c) a polynucleotide comprising the nucleotide sequence of SEQ ID NO:157 from nucleotide 325 to nucleotide 1881;

(d) a polynucleotide comprising the nucleotide sequence of the full-length protein coding sequence of clone hb1041_{—}2 deposited with the ATCC under accession number 98647;

(e) a polynucleotide encoding the full-length protein encoded by the cDNA insert of clone hb1041_{—}2 deposited with the ATCC under accession number 98647;

(f) a polynucleotide comprising the nucleotide sequence of a mature protein coding sequence of clone hb1041_{—}2 deposited with the ATCC under accession number 98647;

(g) a polynucleotide encoding a mature protein encoded by the cDNA insert of clone hb1041_{—}2 deposited with the ATCC under accession number 98647;

(h) a polynucleotide encoding a protein comprising the amino acid sequence of SEQ ID NO:158;

(i) a polynucleotide encoding a protein comprising a fragment of the amino acid sequence of SEQ ID NO: 158 having biological activity, the fragment comprising eight consecutive amino acids of SEQ ID NO:158;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:157 from nucleotide 124 to nucleotide 1881; the nucleotide sequence of SEQ ID NO:157 from nucleotide 325 to nucleotide 1881; the nucleotide sequence of the full-length protein coding sequence of clone hb1041_{—}2 deposited with the ATCC under accession number 98647; or the nucleotide sequence of a mature protein coding sequence of clone hb1041_{—}2 deposited with the ATCC under accession number 98647. In other preferred embodiments, the polynucleotide encodes the full-length or a mature protein encoded by the cDNA insert of clone hb1041_{—}2 deposited with the ATCC under accession number 98647. In further preferred embodiments, the present invention provides a polynucleotide encoding a protein comprising a fragment of the amino acid sequence of SEQ ID NO:158 having biological activity, the fragment preferably comprising eight (more preferably twenty, most preferably thirty) consecutive amino acids of SEQ ID NO: 158, or a polynucleotide encoding a protein comprising a fragment of the amino acid sequence of SEQ ID NO:158 having biological activity, the fragment comprising the amino acid sequence from amino acid 288 to amino acid 297 of SEQ ID NO:158.

Other embodiments provide the gene corresponding to the cDNA sequence of SEQ ID NO:157.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:157, but excluding the poly(A) tail at the 3′ end of SEQ ID NO:157; and

(ab) the nucleotide sequence of the cDNA insert of clone hb1041_{—}2 deposited with the ATCC under accession number 98647;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO: 157, but excluding the poly(A) tail at the 3′ end of SEQ ID NO:157; and

(bb) the nucleotide sequence of the cDNA insert of clone hb1041_{—}2 deposited with the ATCC under accession number 98647;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:157, and extending contiguously from a nucleotide sequence corresponding to the 5′ end of SEQ ID NO:157 to a nucleotide sequence corresponding to the 3′ end of SEQ ID NO:157, but excluding the poly(A) tail at the 3′ end of SEQ ID NO:157. Also preferably the polynucleotide isolated according to the above process comprises a nucleotide sequence corresponding to the cDNA sequence of SEQ ID NO:157 from nucleotide 124 to nucleotide 1881, and extending contiguously from a nucleotide sequence corresponding to the 5′ end of said sequence of SEQ ID NO:157 from nucleotide 124 to nucleotide 1881, to a nucleotide sequence corresponding to the 3′ end of said sequence of SEQ ID NO:157 from nucleotide 124 to nucleotide 1881. Also preferably the polynucleotide isolated according to the above process comprises a nucleotide sequence corresponding to the cDNA sequence of SEQ ID NO:157 from nucleotide 325 to nucleotide 1881, and extending contiguously from a nucleotide sequence corresponding to the 5′ end of said sequence of SEQ ID NO:157 from nucleotide 325 to nucleotide 1881, to a nucleotide sequence corresponding to the 3′ end of said sequence of SEQ ID NO:157 from nucleotide 325 to nucleotide 1881.

In other embodiments, the present invention provides a composition comprising a protein, wherein said protein comprises an amino acid sequence selected from the group consisting of:

(a) the amino acid sequence of SEQ ID NO:158;

(b) fragments of the amino acid sequence of SEQ ID NO:158, each fragment comprising eight consecutive amino acids of SEQ ID NO:158; and

(c) the amino acid sequence encoded by the cDNA insert of clone hb1041_{—}2 deposited with the ATCC under accession number 98647;

the protein being substantially free from other mammalian proteins. Preferably such protein comprises the amino acid sequence of SEQ ID NO:158. In further preferred embodiments, the present invention provides a protein comprising a fragment of the amino acid sequence of SEQ ID NO:158 having biological activity, the fragment preferably comprising eight (more preferably twenty, most preferably thirty) consecutive amino acids of SEQ ID NO:158, or a protein comprising a fragment of the amino acid sequence of SEQ ID NO:158 having biological activity, the fragment comprising the amino acid sequence from amino acid 288 to amino acid 297 of SEQ ID NO:158.

In one embodiment, the present invention provides a composition comprising an isolated polynucleotide selected from the group consisting of:

(a) a polynucleotide comprising the nucleotide sequence of SEQ ID NO:159;

(b) a polynucleotide comprising the nucleotide sequence of SEQ ID NO:159 from nucleotide 163 to nucleotide 1431;

(c) a polynucleotide comprising the nucleotide sequence of the full-length protein coding sequence of clone mh703_{—}1 deposited with the ATCC under accession number 98647;

(d) a polynucleotide encoding the full-length protein encoded by the cDNA insert of clone mh703_{—}1 deposited with the ATCC under accession number 98647;

(e) a polynucleotide comprising the nucleotide sequence of a mature protein coding sequence of clone mh703_{—}1 deposited with the ATCC under accession number 98647;

(f) a polynucleotide encoding a mature protein encoded by the cDNA insert of clone mh703_{—}1 deposited with the ATCC under accession number 98647;

(g) a polynucleotide encoding a protein comprising the amino acid sequence of SEQ ID NO:160;

(h) a polynucleotide encoding a protein comprising a fragment of the amino acid sequence of SEQ ID NO:160 having biological activity, the fragment comprising eight consecutive amino acids of SEQ ID NO:160;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:159fromnucleotide 163 tonucleotide 1431; thenucleotidesequence of the full-length protein coding sequence of clone mh703_{—}1 deposited with the ATCC under accession number 98647; or the nucleotide sequence of a mature protein coding sequence of clone mh703_{—}1 deposited with the ATCC under accession number 98647. In other preferred embodiments, the polynucleotide encodes the full-length or a mature protein encoded by the cDNA insert of clone mh703_{—}1 deposited with the ATCC under accession number 98647. In further preferred embodiments, the present invention provides a polynucleotide encoding a protein comprising a fragment of the amino acid sequence of SEQ ID NO:160 having biological activity, the fragment preferably comprising eight (more preferably twenty, most preferably thirty) consecutive amino acids of SEQ ID NO:160, or a polynucleotide encoding a protein comprising a fragment of the amino acid sequence of SEQ ID NO:160 having biological activity, the fragment comprising the amino acid sequence from amino acid 206 to amino acid 215 of SEQ ID NO:160.

Other embodiments provide the gene corresponding to the cDNA sequence of SEQ ID NO:159.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:159, but excluding the poly(A) tail at the 3′ end of SEQ ID NO:159; and

(ab) the nucleotide sequence of the cDNA insert of clone mh703_{—}1 deposited with the ATCC under accession number 98647;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO: 159, but excluding the poly(A) tail at the 3′ end of SEQ ID NO:159; and

(bb) the nucleotide sequence of the cDNA insert of clone mh703_{—}1 deposited with the ATCC under accession number 98647;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:159, and extending contiguously from a nucleotide sequence corresponding to the 5′ end of SEQ ID NO:159 to a nucleotide sequence corresponding to the 3′ end of SEQ ID NO:159, but excluding the poly(A) tail at the 3′ end of SEQ ID NO:159. Also preferably the polynucleotide isolated according to the above process comprises a nucleotide sequence corresponding to the cDNA sequence of SEQ ID NO:159 from nucleotide 163 to nucleotide 1431, and extending contiguously from a nucleotide sequence corresponding to the 5′ end of said sequence of SEQ ID NO:159 from nucleotide 163 to nucleotide 1431, to a nucleotide sequence corresponding to the 3′ end of said sequence of SEQ ID NO:159 from nucleotide 163 to nucleotide 1431.

In other embodiments, the present invention provides a composition comprising a protein, wherein said protein comprises an amino acid sequence selected from the group consisting of:

(a) the amino acid sequence of SEQ ID NO:160;

(b) fragments of the amino acid sequence of SEQ ID NO:160, each fragment comprising eight consecutive amino acids of SEQ ID NO:160; and

(c) the amino acid sequence encoded by the cDNA insert of clone mh703_{—}1 deposited with the ATCC under accession number 98647;

the protein being substantially free from other mammalian proteins. Preferably such protein comprises the amino acid sequence of SEQ ID NO:160. In further preferred embodiments, the present invention provides a protein comprising a fragment of the amino acid sequence of SEQ ID NO:160 having biological activity, the fragment preferably comprising eight (more preferably twenty, most preferably thirty) consecutive amino acids of SEQ ID NO:160, or a protein comprising a fragment of the amino acid sequence of SEQ ID NO:160 having biological activity, the fragment comprising the amino acid sequence from amino acid 206 to amino acid 215 of SEQ ID NO:160.

In one embodiment, the present invention provides a composition comprising an isolated polynucleotide selected from the group consisting of:

(a) a polynucleotide comprising the nucleotide sequence of SEQ ID NO:161;

(b) a polynucleotide comprising the nucleotide sequence of SEQ ID NO:161 from nucleotide 653 to nucleotide 934;

(c) a polynucleotide comprising the nucleotide sequence of SEQ ID NO:161 from nucleotide 878 to nucleotide 934;

(d) a polynucleotide comprising the nucleotide sequence of the full-length protein coding sequence of clone na461_{—}19 deposited with the ATCC under accession number 98647;

(e) a polynucleotide encoding the full-length protein encoded by the cDNA insert of clone na461_{—}19 deposited with the ATCC under accession number 98647;

(f) a polynucleotide comprising the nucleotide sequence of a mature protein coding sequence of clone na461_{—}19 deposited with the ATCC under accession number 98647;

(g) a polynucleotide encoding a mature protein encoded by the cDNA insert of clone na461_{—}19 deposited with the ATCC under accession number 98647;

(h) a polynucleotide encoding a protein comprising the amino acid sequence of SEQ ID NO:162;

(i) a polynucleotide encoding a protein comprising a fragment of the amino acid sequence of SEQ ID NO:162 having biological activity, the fragment comprising eight consecutive amino acids of SEQ ID NO:162;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:161 fromnucleotide 653 to nucleotide 934; the nucleotide sequence of SEQ ID NO:161 from nucleotide 878 to nucleotide 934; the nucleotide sequence of the full-length protein coding sequence of clone na461_{—}19 deposited with the ATCC under accession number 98647; or the nucleotide sequence of a mature protein coding sequence of clone na461_{—}19 deposited with the ATCC under accession number 98647. In other preferred embodiments, the polynucleotide encodes the full-length or a mature protein encoded by the cDNA insert of clone na461_{—}19 deposited with the ATCC under accession number 98647. In further preferred embodiments, the present invention provides a polynucleotide encoding a protein comprising a fragment of the amino acid sequence of SEQ ID NO:162 having biological activity, the fragment preferably comprising eight (more preferably twenty, most preferably thirty) consecutive amino acids of SEQ ID NO:162, or a polynucleotide encoding a protein comprising a fragment of the amino acid sequence of SEQ ID NO:162 having biological activity, the fragment comprising the amino acid sequence from amino acid 42 to amino acid 51 of SEQ ID NO:162.

Other embodiments provide the gene corresponding to the cDNA sequence of SEQ ID NO:161.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:161, but excluding the poly(A) tail at the 3′ end of SEQ ID NO:161; and

(ab) the nucleotide sequence of the cDNA insert of clone na461_{—}19 deposited with the ATCC under accession number 98647;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:161,but excluding the poly(A) tail at the 3′ end of SEQ ID NO:161; and

(bb) the nucleotide sequence of the cDNA insert of clone na461_{—}19 deposited with the ATCC under accession number 98647;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:161, and extending contiguously from a nucleotide sequence corresponding to the 5′ end of SEQ ID NO:161 to a nucleotide sequence corresponding to the 3′ end of SEQ ID NO:161, but excluding the poly(A) tail at the 3′ end of SEQ ID NO:161. Also preferably the polynucleotide isolated according to the above process comprises a nucleotide sequence corresponding to the cDNA sequence of SEQ ID NO:161 from nucleotide 653 to nucleotide 934, and extending contiguously from a nucleotide sequence corresponding to the 5′ end of said sequence of SEQ ID NO:161 from nucleotide 653 to nucleotide 934, to a nucleotide sequence corresponding to the 3′ end of said sequence of SEQ ID NO:161 from nucleotide 653 to nucleotide 934. Also preferably the polynucleotide isolated according to the above process comprises a nucleotide sequence corresponding to the cDNA sequence of SEQ ID NO:161 from nucleotide 878 to nucleotide 934, and extending contiguously from a nucleotide sequence corresponding to the 5′ end of said sequence of SEQ ID NO:161 from nucleotide 878 to nucleotide 934, to a nucleotide sequence corresponding to the 3′ end of said sequence of SEQ ID NO:161 from nucleotide 878 to nucleotide 934.

In other embodiments, the present invention provides a composition comprising a protein, wherein said protein comprises an amino acid sequence selected from the group consisting of:

(a) the amino acid sequence of SEQ ID NO:162;

(b) fragments of the amino acid sequence of SEQ ID NO:162, each fragment comprising eight consecutive amino acids of SEQ ID NO:162; and

(c) the amino acid sequence encoded by the cDNA insert of clone na461_{—}19 deposited with the ATCC under accession number 98647;

the protein being substantially free from other mammalian proteins. Preferably such protein comprises the amino acid sequence of SEQ ID NO:162. In further preferred embodiments, the present invention provides a protein comprising a fragment of the amino acid sequence of SEQ ID NO:162 having biological activity, the fragment preferably comprising eight (more preferably twenty, most preferably thirty) consecutive amino acids of SEQ ID NO:162, or a protein comprising a fragment of the amino acid sequence of SEQ ID NO:162 having biological activity, the fragment comprising the amino acid sequence from amino acid 42 to amino acid 51 of SEQ ID NO: 162.

In one embodiment, the present invention provides a composition comprising an isolated polynucleotide selected from the group consisting of:

(a) a polynucleotide comprising the nucleotide sequence of SEQ ID NO:163;

(b) a polynucleotide comprising the nucleotide sequence of SEQ ID NO:163 from nucleotide 72 to nucleotide 1319;

(c) a polynucleotide comprising the nucleotide sequence of SEQ ID NO:163 from nucleotide 1071 to nucleotide 1319;

(d) a polynucleotide comprising the nucleotide sequence of the full-length protein coding sequence of clone na492_{—}2 deposited with the ATCC under accession number 98647;

(e) a polynucleotide encoding the full-length protein encoded by the cDNA insert of clone na492_{—}2 deposited with the ATCC under accession number 98647;

(f) a polynucleotide comprising the nucleotide sequence of a mature protein coding sequence of clone na492_{—}2 deposited with the ATCC under accession number 98647;

(g) a polynucleotide encoding a mature protein encoded by the cDNA insert of clone na492_{—}2 deposited with the ATCC under accession number 98647;

(h) a polynucleotide encoding a protein comprising the amino acid sequence of SEQ ID NO:164;

(i) a polynucleotide encoding a protein comprising a fragment of the amino acid sequence of SEQ ID NO:164 having biological activity, the fragment comprising eight consecutive amino acids of SEQ ID NO:164;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:163 from nucleotide 72 to nucleotide 1319; the nucleotide sequence of SEQ ID NO:163 from nucleotide 1071 to nucleotide 1319; the nucleotide sequence of the full-length protein coding sequence of clone na492_{—}2 deposited with the ATCC under accession number 98647; or the nucleotide sequence of a mature protein coding sequence of clone na492_{—}2 deposited with the ATCC under accession number 98647. In other preferred embodiments, the polynucleotide encodes the full-length or a mature protein encoded by the cDNA insert of clone na492_{—}2 deposited with the ATCC under accession number 98647. In further preferred embodiments, the present invention provides a polynucleotide encoding a protein comprising a fragment of the amino acid sequence of SEQ ID NO:164 having biological activity, the fragment preferably comprising eight (more preferably twenty, most preferably thirty) consecutive amino acids of SEQ ID NO:164, or a polynucleotide encoding a protein comprising a fragment of the amino acid sequence of SEQ ID NO:164 having biological activity, the fragment comprising the amino acid sequence from amino acid 202 to amino acid 211 of SEQ ID NO:164.

Other embodiments provide the gene corresponding to the cDNA sequence of SEQ ID NO:163.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO: 163, but excluding the poly(A) tail at the 3′ end of SEQ ID NO:163; and

(ab) the nucleotide sequence of the cDNA insert of clone na492_{—}2 deposited with the ATCC under accession number 98647;

(ii) hybridizing said probe(s) to human genomic DNA in conditions at least as stringent as 4×SSC at 65 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:163, but excluding the poly(A) tail at the 3′ end of SEQ ID NO:163; and

(bb) the nucleotide sequence of the cDNA insert of clone na492_{—}2 deposited with the ATCC under accession number 98647;

(ii) hybridizing said primer(s) to human genomic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:163, and extending contiguously from a nucleotide sequence corresponding to the 5′ end of SEQ ID NO:163 to a nucleotide sequence corresponding to the 3′ end of SEQ ID NO:163, but excluding the poly(A) tail at the 3′ end of SEQ ID NO:163. Also preferably the polynucleotide isolated according to the above process comprises a nucleotide sequence corresponding to the cDNA sequence of SEQ ID NO:163 from nucleotide 72 to nucleotide 1319, and extending contiguously from a nucleotide sequence corresponding to the 5′ end of said sequence of SEQ ID NO:163 from nucleotide 72 to nucleotide 1319, to a nucleotide sequence corresponding to the 3′ end of said sequence of SEQ ID NO:163 from nucleotide 72 to nucleotide 1319. Also preferably the polynucleotide isolated according to the above process comprises a nucleotide sequence corresponding to the cDNA sequence of SEQ ID NO:163 from nucleotide 1071 to nucleotide 1319, and extending contiguously from a nucleotide sequence corresponding to the 5′ end of said sequence of SEQ ID NO:163 from nucleotide 1071 to nucleotide 1319, to a nucleotide sequence corresponding to the 3′ end of said sequence of SEQ ID NO:163 from nucleotide 1071 to nucleotide 1319.

In other embodiments, the present invention provides a composition comprising a protein, wherein said protein comprises an amino acid sequence selected from the group consisting of:

(a) the amino acid sequence of SEQ ID NO:164;

(b) fragments of the amino acid sequence of SEQ ID NO:164, each fragment comprising eight consecutive amino acids of SEQ ID NO:164; and

(c) the amino acid sequence encoded by the cDNA insert of clone na492_{—}2 deposited with the ATCC under accession number 98647;

the protein being substantially free from other mammalian proteins. Preferably such protein comprises the amino acid sequence of SEQ ID NO:164. In further preferred embodiments, the present invention provides a protein comprising a fragment of the amino acid sequence of SEQ ID NO:164 having biological activity, the fragment preferably comprising eight (more preferably twenty, most preferably thirty) consecutive amino acids of SEQ ID NO:164, or a protein comprising a fragment of the anino acid sequence of SEQ ID NO:164 having biological activity, the fragment comprising the amino acid sequence from amino acid 202 to amino acid 211 of SEQ ID NO:164.

In one embodiment, the present invention provides a composition comprising an isolated polynucleotide selected from the group consisting of:

(a) a polynucleotide comprising the nucleotide sequence of SEQ ID NO:165;

(b) a polynucleotide comprising the nucleotide sequence of SEQ ID NO:165 from nucleotide 2848 to nucleotide 3048;

(c) a polynucleotide comprising the nucleotide sequence of SEQ ID NO:165 from nucleotide 3004 to nucleotide 3048;

(d) a polynucleotide comprising the nucleotide sequence of the full-length protein coding sequence of clone na669_{—}10 deposited with the ATCC under accession number 98647;

(e) a polynucleotide encoding the full-length protein encoded by the cDNA insert of clone na669_{—}10 deposited with the ATCC under accession number 98647;

(f) a polynucleotide comprising the nucleotide sequence of a mature protein coding sequence of clone na669_{—}10 deposited with the ATCC under accession number 98647;

(g) a polynucleotide encoding a mature protein encoded by the cDNA insert of clone na669_{—}10 depositedwith the ATCC under accessionnumber 98647;

(h) a polynucleotide encoding a protein comprising the amino acid sequence of SEQ ID NO:166;

(i) a polynucleotide encoding a protein comprising a fragment of the amino acid sequence of SEQ ID NO:166 having biological activity, the fragment comprising eight consecutive amino acids of SEQ ID NO:166;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:165 from nucleotide 2848 to nucleotide 3048; the nucleotide sequence of SEQ ID NO:165 from nucleotide 3004 to nucleotide 3048; the nucleotide sequence of the full-length protein coding sequence of clone na669_{—}10 deposited with the ATCC under accession number 98647; or the nucleotide sequence of a mature protein coding sequence of clone na669_{—}10 deposited with the ATCC under accession number 98647. In other preferred embodiments, the polynucleotide encodes the full-length or a mature protein encoded by the cDNA insert of clone na669_{—}10 deposited with the ATCC under accession number 98647. In yet other preferred embodiments, the present invention provides a polynucleotide encoding a protein comprising the amino acid sequence of SEQ ID NO:166 from amino acid 5 to amino acid 62. In further preferred embodiments, the present invention provides a polynucleotide encoding a protein comprising a fragment of the amino acid sequence of SEQ ID NO:166 having biological activity, the fragment preferably comprising eight (more preferably twenty, most preferably thirty) consecutive amino acids of SEQ ID NO:166, or a polynucleotide encoding a protein comprising a fragment of the amino acid sequence of SEQ ID NO:166 having biological activity, the fragment comprising the amino acid sequence from amino acid 28 to amino acid 37 of SEQ ID NO:166.

Other embodiments provide the gene corresponding to the cDNA sequence of SEQ ID NO:165.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO: 165, but excluding the poly(A) tail at the 3′ end of SEQ ID NO:165; and

(ab) the nucleotide sequence of the cDNA insert of clone na669_{—}10 deposited with the ATCC under accession number 98647;

(ii) hybridizing said probe(s) to human genomic DNA in conditions at least as stringent as 4×SSC at 65 degrees C; and

(iii) isolating the DNA polynucleotides detected wth the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:165, but excluding the poly(A) tail at the 3′ end of SEQ ID NO:165; and

(bb) the nucleotide sequence of the cDNA insert of clone na669_{—}10 deposited with the ATCC under accession number 98647;

(ii) hybridizing said primer(s) to human genornic DNA in conditions at least as stringent as 4×SSC at 65 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:165, and extending contiguously from a nucleotide sequence corresponding to the 5′ end of SEQ ID NO:165 to a nucleotide sequence corresponding to the 3′ end of SEQ ID NO:165, but excluding the poly(A) tail at the 3′ end of SEQ ID NO:165. Also preferably the polynucleotide isolated according to the above process comprises a nucleotide sequence corresponding to the cDNA sequence of SEQ ID NO:165 from nucleotide 2848 to nucleotide 3048, and extending contiguously from a nucleotide sequence corresponding to the 5′ end of said sequence of SEQ ID NO:165 from nucleotide 2848 to nucleotide 3048, to a nucleotide sequence corresponding to the 3′ end of said sequence of SEQ ID NO:165 from nucleotide 2848 to nucleotide 3048. Also preferably the polynucleotide isolated according to the above process comprises a nucleotide sequence corresponding to the cDNA sequence of SEQ ID NO:165 from nucleotide 3004 to nucleotide 3048, and extending contiguously from a nucleotide sequence corresponding to the 5′ end of said sequence of SEQ ID NO:165 from nucleotide 3004 to nucleotide 3048, to a nucleotide sequence corresponding to the 3′ end of said sequence of SEQ ID NO:165 from nucleotide 3004 to nucleotide 3048.

In other embodiments, the present invention provides a composition comprising a protein, wherein said protein comprises an amino acid sequence selected from the group consisting of:

(a) the amino acid sequence of SEQ ID NO:166;

(b) the amino acid sequence of SEQ ID NO:166 from amino acid 5 to amino acid 62;

(c) fragments of the amino acid sequence of SEQ ID NO:166, each fragment comprising eight consecutive amino acids of SEQ ID NO:166; and

(d) the amino acid sequence encoded by the cDNA insert of clone na669_{—}10 deposited with the ATCC under accession number 98647;

the protein being substantially free from other mammalian proteins. Preferably such protein comprises the amino acid sequence of SEQ ID NO:166 or the amino acid sequence of SEQ ID NO:166 from amino acid 5 to amino acid 62. In further preferred embodiments, the present invention provides a protein comprising a fragment of the amino acid sequence of SEQ ID NO:166 having biological activity, the fragment preferably comprising eight (more preferably twenty, most preferably thirty) consecutive amino acids of SEQ ID NO:166, or a protein comprising a fragment of the amino acid sequence of SEQ ID NO:166 having biological activity, the fragment comprising the amino acid sequence from amino acid 28 to amino acid 37 of SEQ ID NO:166.

In one embodiment, the present invention provides a composition comprising an isolated polynucleotide selected from the group consisting of:

(a) a polynucleotide comprising the nucleotide sequence of SEQ ID NO:167;

(b) a polynucleotide comprising the nucleotide sequence of SEQ ID NO:167 from nucleotide 185 to nucleotide 1678;

(c) a polynucleotide comprising the nucleotide sequence of SEQ ID NO:167 from nucleotide 482 to nucleotide 1678;

(d) a polynucleotide comprising the nucleotide sequence of the full-length protein coding sequence of clone co821_{—}31 deposited with the ATCC under accession number 98663;

(e) a polynucleotide encoding the full-length protein encoded by the cDNA insert of clone co821_{—}31 deposited with the ATCC under accession number 98663;

(f) a polynucleotide comprising the nucleotide sequence of a mature protein coding sequence of clone co821_{—}31 deposited with the ATCC under accession number 98663;

(g) a polynucleotide encoding a mature protein encoded by the cDNA insert of clone co821_{—}31 deposited with the ATCC under accession number 98663;

(h) a polynucleotide encoding a protein comprising the amino acid sequence of SEQ ID NO:168;

(i) a polynucleotide encoding a protein comprising a fragment of the amino acid sequence of SEQ ID NO:168 having biological activity, the fragment comprising eight contiguous amino acids of SEQ ID NO:168;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:167.

Preferably, such polynucleotide comprises the nucleotide sequence of SEQ ID NO:167 from nucleotide 185 to nucleotide 1678; the nucleotide sequence of SEQ ID NO:167 from nucleotide 482 to nucleotide 1678; the nucleotide sequence of the fulllength protein coding sequence of clone co821_{—}31 deposited with the ATCC under accession number 98663; or the nucleotide sequence of a mature protein coding sequence of clone co821_{—}31 deposited with the ATCC under accession number 98663. In other preferred embodiments, the polynucleotide encodes the full-length or a mature protein encoded by the cDNA insert of clone co821_{—}31 deposited with the ATCC under accession number 98663. In further preferred embodiments, the present invention provides a polynucleotide encoding a protein comprising a fragment of the amino acid sequence of SEQ ID NO:168 having biological activity, the fragment preferably comprising eight (more preferably twenty, most preferably thirty) contiguous amino acids of SEQ ID NO:168, or a polynucleotide encoding a protein comprising a fragment of the amino acid sequence of SEQ ID NO:168 having biological activity, the fragment comprising the amino acid sequence from amino acid 244 to amino acid 253 of SEQ ID NO:168.

Other embodiments provide the gene corresponding to the cDNA sequence of SEQ ID NO:167.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:167, but excluding the poly(A) tail at the 3′ end of SEQ ID NO:167; and

(ab) the nucleotide sequence of the cDNA insert of clone co821_{—}31 deposited with the ATCC under accession number 98663;

(ii) hybridizing said probe(s) to human genomic DNA in conditions at least as stringent as 4×SSC at 50 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:167, but excluding the poly(A) tail at the 3′ end of SEQ ID NO:167; and

(bb) the nucleotide sequence of the cDNA insert of clone co821_{—}31 deposited with the ATCC under accession number 98663;

(ii) hybridizing said primer(s) to human genomic DNA in conditions at least as stringent as 4×SSC at 50 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:167, and extending contiguously from a nucleotide sequence corresponding to the 5′ end of SEQ ID NO:167 to a nucleotide sequence corresponding to the 3′ end of SEQ ID NO:167, but excluding the poly(A) tail at the 3′ end of SEQ ID NO:167. Also preferably the polynucleotide isolated according to the above process comprises a nucleotide sequence corresponding to the cDNA sequence of SEQ ID NO:167 from nucleotide 185 to nucleotide 1678, and extending contiguously from a nucleotide sequence corresponding to the 5′ end of said sequence of SEQ ID NO:167 from nucleotide 185 to nucleotide 1678, to a nucleotide sequence corresponding to the 3′ end of said sequence of SEQ ID NO:167 from nucleotide 185 to nucleotide 1678. Also preferably the polynucleotide isolated according to the above process comprises a nucleotide sequence corresponding to the cDNA sequence of SEQ ID NO:167 from nucleotide 482 to nucleotide 1678, and extending contiguously from a nucleotide sequence corresponding to the 5′ end of said sequence of SEQ ID NO:167 from nucleotide 482 to nucleotide 1678, to a nucleotide sequence corresponding to the 3′ end of said sequence of SEQ ID NO:167 from nucleotide 482 to nucleotide 1678.

In other embodiments, the present invention provides a composition comprising a protein, wherein said protein comprises an amino acid sequence selected from the group consisting of:

(a) the amino acid sequence of SEQ ID NO:168;

(b) a fragment of the amino acid sequence of SEQ ID NO:168, the fragment comprising eight contiguous amino acids of SEQ ID NO:168; and

(c) the amino acid sequence encoded by the cDNA insert of clone co821_{—}31 deposited with the ATCC under accession number 98663;

the protein being substantially free from other mammalian proteins. Preferably such protein comprises the amino acid sequence of SEQ ID NO:168. In further preferred embodiments, the present invention provides a protein comprising a fragment of the amino acid sequence of SEQ ID NO:168 having biological activity, the fragment preferably comprising eight (more preferably twenty, most preferably thirty) contiguous amino acids of SEQ ID NO:168, or a protein comprising a fragment of the amino acid sequence of SEQ ID NO:168 having biological activity, the fragment comprising the amino acid sequence from amino acid 244 to amino acid 253 of SEQ ID NO:168.

In one embodiment, the present invention provides a composition comprising an isolated polynucleotide selected from the group consisting of:

(a) a polynucleotide comprising the nucleotide sequence of SEQ ID NO:169;

(b) a polynucleotide comprising the nucleotide sequence of SEQ ID NO:169 from nucleotide 176 to nucleotide 754;

(c) a polynucleotide comprising the nucleotide sequence of SEQ ID NO:169 from nucleotide 425 to nucleotide 754;

(d) a polynucleotide comprising the nucleotide sequence of the full-length protein coding sequence of clone dk329_{—}1 deposited with the ATCC under accession number 98663;

(e) a polynucleotide encoding the full-length protein encoded by the cDNA insert of clone dk329_{—}1 deposited with the ATCC under accession number 98663;

(f) a polynucleotide comprising the nucleotide sequence of a mature protein coding sequence of clone dk329_{—}1 deposited with the ATCC under accession number 98663;

(g) a polynucleotide encoding a mature protein encoded by the cDNA insert of clone dk329_{—}1 deposited with the ATCC under accession number 98663;

(h) a polynucleotide encoding a protein comprising the amino acid sequence of SEQ ID NO:170;

(i) a polynucleotide encoding a protein comprising a fragment of the amino acid sequence of SEQ ID NO:170 having biological activity, the fragment comprising eight contiguous amino acids of SEQ ID NO:170;

(j) a polynucleotide which is an allelic variant of a polynucleotide of (a)-(g) above;

(k) a polynucleotide which encodes a species homologue of the protein of (h) or (i) above;

(1) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:169.

Preferably, such polynucleotide comprises the nucleotide sequence of SEQ ID NO:169 fromnucleotide 176 to nucleotide 754; the nucleotide sequence of SEQ ID NO:169 from nucleotide 425 to nucleotide 754; the nucleotide sequence of the full-length protein coding sequence of clone dk329_{—}1 deposited with the ATCC under accession number 98663; or the nucleotide sequence of a mature protein coding sequence of clone dk329_{—}1 deposited with the ATCC under accession number 98663. In other preferred embodiments, the polynucleotide encodes the full-length or a mature protein encoded by the cDNA insert of clone dk329_{—}1 deposited with the ATCC under accession number 98663. In further preferred embodiments, the present invention provides a polynucleotide encoding a protein comprising a fragment of the amino acid sequence of SEQ ID NO:170 having biological activity, the fragment preferably comprising eight (more preferably twenty, most preferably thirty) contiguous amino acids of SEQ ID NO:170, or a polynucleotide encoding a protein comprising a fragment of the amino acid sequence of SEQ ID NO:170 having biological activity, the fragment comprising the amino acid sequence from amino acid 91 to amino acid 100 of SEQ ID NO:170.

Other embodiments provide the gene corresponding to the cDNA sequence of SEQ ID NO:169.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:169, but excluding the poly(A) tail at the 3′ end of SEQ ID NO:169; and

(ab) the nucleotide sequence of the cDNA insert of clone dk329_{—}1 deposited with the ATCC under accession number 98663;

(ii) hybridizing said probe(s) to human genomic DNA in conditions at least as stringent as 4×SSC at 50 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:169, but excluding the poly(A) tail at the 3′ end of SEQ ID NO:169; and

(bb) the nucleotide sequence of the cDNA insert of clone dk329_{—}1 deposited with the ATCC under accession number 98663;

(ii) hybridizing said primer(s) to human genornic DNA in conditions at least as stringent as 4×SSC at 50 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:169, and extending contiguously from a nucleotide sequence corresponding to the 5′ end of SEQ ID NO:169 to a nucleotide sequence corresponding to the 3′ end of SEQ ID NO:169, but excluding the poly(A) tail at the 3′ end of SEQ ID NO:169. Also preferably the polynucleotide isolated according to the above process comprises a nucleotide sequence corresponding to the cDNA sequence of SEQ ID NO:169 from nucleotide 176 to nucleotide 754, and extending contiguously from a nucleotide sequence corresponding to the 5′ end of said sequence of SEQ ID NO:169 from nucleotide 176 to nucleotide 754, to a nucleotide sequence corresponding to the 3′ end of said sequence of SEQ ID NO:169 from nucleotide 176 to nucleotide 754. Also preferably the polynucleotide isolated according to the above process comprises a nucleotide sequence corresponding to the cDNA sequence of SEQ ID NO:169 from nucleotide 425 to nucleotide 754, and extending contiguously from a nucleotide sequence corresponding to the 5′ end of said sequence of SEQ ID NO:169 from nucleotide 425 to nucleotide 754, to a nucleotide sequence corresponding to the 3′ end of said sequence of SEQ ID NO:169 from nucleotide 425 to nucleotide 754.

In other embodiments, the present invention provides a composition comprising a protein, wherein said protein comprises an amino acid sequence selected from the group consisting of:

(a) the amino acid sequence of SEQ ID NO:170;

(b) a fragment of the amino acid sequence of SEQ ID NO:170, the fragment comprising eight contiguous amino acids of SEQ ID NO:170; and

(c) the amino acid sequence encoded by the cDNA insert of clone dk329_{—}1 deposited with the ATCC under accession number 98663;

the protein being substantially free from other mammalian proteins. Preferably such protein comprises the amino acid sequence of SEQ ID NO:170. In further preferred embodiments, the present invention provides a protein comprising a fragment of the amino acid sequence of SEQ ID NO:170 having biological activity, the fragment preferably comprising eight (more preferably twenty, most preferably thirty) contiguous amino acids of SEQ ID NO:170, or a protein comprising a fragment of the amino acid sequence of SEQ ID NO:170 having biological activity, the fragment comprising the amino acid sequence from amino acid 91 to amino acid 100 of SEQ ID NO:170.

In one embodiment, the present invention provides a composition comprising an isolated polynucleotide selected from the group consisting of:

(a) a polynucleotide comprising the nucleotide sequence of SEQ ID NO:171;

(b) a polynucleotide comprising the nucleotide sequence of SEQ ID NO:171 from nucleotide 190 to nucleotide 1449;

(c) a polynucleotide comprising the nucleotide sequence of SEQ ID NO:171 from nucleotide 913 to nucleotide 1449;

(d) a polynucleotide comprising the nucleotide sequence of the full-length protein coding sequence of clone fx317_{—}11 deposited with the ATCC under accession number 98663;

(e) a polynucleotide encoding the full-length protein encoded by the cDNA insert of clone fx317_{—}11 deposited with the ATCC under accession number 98663;

(f) a polynucleotide comprising the nucleotide sequence of a mature protein coding sequence of clone fx317_{—}11 deposited with the ATCC under accession number 98663;

(g) a polynucleotide encoding a mature protein encoded by the cDNA insert of clone fx317_{—}11 deposited with the ATCC under accession number 98663;

(h) a polynucleotide encoding a protein comprising the amnino acid sequence of SEQ ID NO:172;

(i) a polynucleotide encoding a protein comprising a fragment of the amino acid sequence of SEQ ID NO:172 having biological activity, the fragment comprising eight contiguous amino acids of SEQ ID NO:172;

(j) a polynucleotide which is an allelic variant of a polynucleotide of (a)-(g) above;

(k) a pglynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:171.

Preferably, such polynucleotide comprises the nucleotide sequence of SEQ ID NO:171 from nucleotide 190 to nucleotide 1449; the nucleotide sequence of SEQ ID NO:171 from nucleotide 913 to nucleotide 1449; the nucleotide sequence of the full-length protein coding sequence of clone fx317_{—}11 deposited with the ATCC under accession number 98663; or the nucleotide sequence of a mature protein coding sequence of clone fx317_{—}11 deposited with the ATCC under accession number 98663. In other preferred embodiments, the polynucleotide encodes the full-length or a mature protein encoded by the cDNA insert of clone fx317_{—}11 deposited with the ATCC under accession number 98663. In further preferred embodiments, the present invention provides a polynucleotide encoding a protein comprising a fragment of the amino acid sequence of SEQ ID NO:172 having biological activity, the fragment preferably comprising eight (more preferably twenty, most preferably thirty) contiguous amino acids of SEQ ID NO:172, or a polynucleotide encoding a protein comprising a fragment of the amino acid sequence of SEQ ID NO:172 having biological activity, the fragment comprising the amino acid sequence from amino acid 205 to amino acid 214 of SEQ ID NO:172.

Other embodiments provide the gene corresponding to the cDNA sequence of SEQ ID NO:171.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:171,but excluding the poly(A) tail at the 3′ end of SEQ ID NO:171; and

(ab) the nucleotide sequence of the cDNA insert of clone fx317_{—}11 depositedwiththe ATCC underaccession number 98663;

(ii) hybridizing said probe(s) to human genomic DNA in conditions at least as stringent as 4×SSC at 50 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:171, but excluding the poly(A) tail at the 3′ end of SEQ ID NO:171; and

(bb) the nucleotide sequence of the cDNA insert of clone fx317_{—}11 deposited with the ATCC under accession number 98663;

(ii) hybridizing said primer(s) to human genomic DNA in conditions at least as stringent as 4×SSC at 50 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:171, and extending contiguously from a nucleotide sequence corresponding to the 5′ end of SEQ ID NO:171 to a nucleotide sequence corresponding to the 3′ end of SEQ ID NO:171, but excluding the poly(A) tail at the 3′ end of SEQ ID NO:171. Also preferably the polynucleotide isolated according to the above process comprises a nucleotide sequence corresponding to the cDNA sequence of SEQ ID NO:171 from nucleotide 190 to nucleotide 1449, and extending contiguously from a nucleotide sequence corresponding to the 5′ end of said sequence of SEQ ID NO:171 from nucleotide 190 to nucleotide 1449, to a nucleotide sequence corresponding to the 3′ end of said sequence of SEQ ID NO:171 from nucleotide 190 to nucleotide 1449. Also preferably the polynucleotide isolated according to the above process comprises a nucleotide sequence corresponding to the cDNA sequence of SEQ ID NO:171 fromnucleotide 913 to nucleotide 1449, and extending contiguously from a nucleotide sequence corresponding to the 5′ end of said sequence of SEQ ID NO:171 from nucleotide 913 to nucleotide 1449, to a nucleotide sequence corresponding to the 3′ end of said sequence of SEQ ID NO:171 from nucleotide 913 to nucleotide 1449.

In other embodiments, the present invention provides a composition comprising a protein, wherein said protein comprises an amino acid sequence selected from the group consisting of:

(a) the amino acid sequence of SEQ ID NO:172;

(b) a fragment of the amino acid sequence of SEQ ID NO:172, the fragment comprising eight contiguous amino acids of SEQ ID NO:172; and

(c) the amino acid sequence encoded by the cDNA insert of clone fx317_{—}11 deposited with the ATCC under accession number 98663;

the protein being substantially free from other mammalian proteins. Preferably such protein comprises the amino acid sequence of SEQ ID NO:172. In further preferred embodiments, the present invention provides a protein comprising a fragment of the amino acid sequence of SEQ ID NO:172 having biological activity, the fragment preferably comprising eight (more preferably twenty, most preferably thirty) contiguous amino acids of SEQ ID NO:172, or a protein comprising a fragment of the amino acid sequence of SEQ ID NO:172 having biological activity, the fragment comprising the amino acid sequence from amino acid 205 to amino acid 214 of SEQ ID NO:172.

In one embodiment, the present invention provides a composition comprising an isolated polynucleotide selected from the group consisting of:

(a) a polynucleotide comprising the nucleotide sequence of SEQ ID NO:173;

(b) a polynucleotide comprising the nucleotide sequence of SEQ ID NO:173 from nucleotide 51 to nucleotide 1202;

(c) a polynucleotide comprising the nucleotide sequence of the full-length protein coding sequence of clone lp547_{—}4 deposited with the ATCC under accession number 98663;

(d) a polynucleotide encoding the full-length protein encoded by the cDNA insert of clone lp547_{—}4 deposited with the ATCC under accession number 98663;

(e) a polynucleotide comprising the nucleotide sequence of a mature protein coding sequence of clone lp547_{—}4 deposited with the ATCC under accession number 98663;

(f) a polynucleotide encoding a mature protein encoded by the cDNA insert of clone lp547_{—}4 deposited with the ATCC under accession number 98663;

(g) a polynucleotide encoding a protein comprising the amino acid sequence of SEQ ID NO:174;

(h) a polynucleotide encoding a protein comprising a fragment of the amino acid sequence of SEQ ID NO:174 having biological activity, the fragment comprising eight contiguous amino acids of SEQ ID NO:174;

(i) a polynucleotide which is an allelic variant of a polynucleotide of (a)-(f) above;

(j) a polynucleotide which encodes a species homologue of the protein of (g) or (h) above;

(k) a polynucleotide that hybridizes under stringent conditions to any one of the polynucleotides specified in (a)-(h); and

(l) a polynucleotide that hybridizes under stringent conditions to any one of the polynucleotides specified in (a)-(h) and that has a length that is at least 25% of the length of SEQ ID NO:173.

Preferably, such polynucleotide comprises the nucleotide sequence of SEQ ID NO:173 from nucleotide 51 to nucleotide 1202; the nucleotide sequence of the full-length protein coding sequence of clone lp547_{—}4 deposited with the ATCC under accession number 98663; or the nucleotide sequence of a mature protein coding sequence of clone lp547_{—}4 deposited with the ATCC under accession number 98663. In other preferred embodiments, the polynucleotide encodes the full-length or a mature protein encoded by the cDNA insert of clone lp547_{—}4 deposited with the ATCC under accession number 98663. In further preferred embodiments, the present invention provides a polynucleotide encoding a protein comprising a fragment of the amino acid sequence of SEQ ID NO:174 having biological activity, the fragment preferably comprising eight (more preferably twenty, most preferably thirty) contiguous amino acids of SEQ ID NO:174, or a polynucleotide encoding a protein comprising a fragment of the amnino acid sequence of SEQ ID NO:174 having biological activity, the fragment comprising the amino acid sequence from amino acid 187 to amino acid 196 of SEQ ID NO:174.

Other embodiments provide the gene corresponding to the cDNA sequence of SEQ ID NO:173.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO: 173, but excluding the poly(A) tail at the 3′ end of SEQ ID NO:173; and

(ab) the nucleotide sequence of the cDNA insert of clone lp547_{—}4 deposited with the ATCC under accession number 98663;

(ii) hybridizing said probe(s) to human genomic DNA in conditions at least as stringent as 4×SSC at 50 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:173, but exluding the poly(A) tail at the 3′ end of SEQ ID NO:173; and

(bb) the nucleotide sequence of the cDNA insert of clone lp547_{—}4 deposited with the ATCC under accession number 98663;

(ii) hybridizing said primer(s) to human genomic DNA in conditions at least as stringent as 4×SSC at 50 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:173, and extending contiguously from a nucleotide sequence corresponding to the 5′ end of SEQ ID NO:173 to a nucleotide sequence corresponding to the 3′ end of SEQ ID NO:173, but excluding the poly(A) tail at the 3′ end of SEQ ID NO:173. Also preferably the polynucleotide isolated according to the above process comprises a nucleotide sequence corresponding to the cDNA sequence of SEQ ID NO:173 from nucleotide 51 to nucleotide 1202, and extending contiguously from a nucleotide sequence corresponding to the 5′ end of said sequence of SEQ ID NO:173 from nucleotide 51 to nucleotide 1202, to a nucleotide sequence corresponding to the 3′ end of said sequence of SEQ ID NO:173 from nucleotide 51 to nucleotide 1202.

In other embodiments, the present invention provides a composition comprising a protein, wherein said protein comprises an amino acid sequence selected from the group consisting of:

(a) the amino acid sequence of SEQ ID NO:174;

(b) a fragment of the amino acid sequence of SEQ ID NO:174, the fragment comprising eight contiguous amino acids of SEQ ID NO:174; and

(c) the amino acid sequence encoded by the cDNA insert of clone lp547_{—}4 deposited with the ATCC under accession number 98663;

the protein being substantially free from other mammalian proteins. Preferably such protein comprises the amino acid sequence of SEQ ID NO:174. In further preferred embodiments, the present invention provides a protein comprising a fragment of the amino acid sequence of SEQ ID NO:174 having biological activity, the fragment preferably comprising eight (more preferably twenty, most preferably thirty) contiguous amino acids of SEQ ID NO:174, or a protein comprising a fragment of the amino acid sequence of SEQ ID NO:174 having biological activity, the fragment comprising the L amino acid sequence from amino acid 187 to amino acid 196 of SEQ ID NO:174.

In one embodiment, the present invention provides a composition comprising an isolated polynucleotide selected from the group consisting of:

(a) a polynucleotide comprising the nucleotide sequence of SEQ ID NO:175;

(b) a polynucleotide comprising the nucleotide sequence of SEQ ID NO:175 from nucleotide 61 to nucleotide 2559;

(c) a polynucleotide comprising the nucleotide sequence of SEQ ID NO:175 from nucleotide 904 to nucleotide 2559;

(d) a polynucleotide comprising the nucleotide sequence of the full-length protein coding sequence of clone lv310_{—}7 deposited with the ATCC under accession number 98663;

(e) a polynucleotide encoding the full-length protein encoded by the cDNA insert of clone lv310_{—}7 deposited with the ATCC under accession number 98663;

(f) a polynucleotide comprising the nucleotide sequence of a mature protein coding sequence of clone lv310_{—}7 deposited with the ATCC under accession number 98663;

(g) a polynucleotide encoding a mature protein encoded by the cDNA insert of clone lv310_{—}7 deposited with the ATCC under accession number 98663;

(h) a polynucleotide encoding a protein comprising the amino acid sequence of SEQ ID NO:176;

(i) a polynucleotide encoding a protein comprising a fragment of the amino acid sequence of SEQ ID NO:176 having biological activity, the fragment comprising eight contiguous amino acids of SEQ ID NO:176;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:175.

Preferably, such polynucleotide comprises the nucleotide sequence of SEQ ID NO:175 from nucleotide 61 to nucleotide 2559; the nucleotide sequence of SEQ ID NO:175 from nudeotide 904 to nucleotide 2559; the nucleotide sequence of the full-length protein coding sequence of clone lv310_{—}7 deposited with the ATCC under accession number 98663; or the nucleotide sequence of a mature protein coding sequence of clone lv310_{—}7 deposited with the ATCC under accession number 98663. In other preferred embodiments, the polynucleotide encodes the full-length or a mature protein encoded by the cDNA insert of clone lv310_{—}7 deposited with the ATCC under accession number 98663. In further preferred embodiments, the present invention provides a polynucleotide encoding a protein comprising a fragment of the amino acid sequence of SEQ ID NO:176 having biological activity, the fragment preferably comprising eight (more preferably twenty, most preferably thirty) contiguous amino acids of SEQ ID NO:176, or a polynucleotide encoding a protein comprising a fragment of the amino acid sequence of SEQ ID NO:176 having biological activity, the fragment comprising the amino acid sequence from amino acid 411 to amino acid 420 of SEQ ID NO:176.

Other embodiments provide the gene corresponding to the cDNA sequence of SEQ ID NO:175.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:175, but excluding the poly(A) tail at the 3′ end of SEQ ID NO:175; and

(ab) the nucleotide sequence of the cDNA insert of clone lv310_{—}7 deposited with the ATCC under accession number 98663;

(ii) hybridizing said probe(s) to human genomic DNA in conditions at least as stringent as 4×SSC at 50 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:175, but excluding the poly(A) tail at the 3′ end of SEQ ID NO:175; and

(bb) the nucleotide sequence of the cDNA insert of clone lv310_{—}7 deposited with the ATCC under accession number 98663;

(ii) hybridizing said primer(s) to human genomic DNA in conditions at least as stringent as 4×SSC at 50 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:175, and extending contiguously from a nucleotide sequence corresponding to the 5′ end of SEQ ID NO:175 to a nucleotide sequence corresponding to the 3′ end of SEQ ID NO:175, but excluding the poly(A) tail at the 3′ end of SEQ ID NO:175. Also preferably the polynucleotide isolated according to the above process comprises a nucleotide sequence corresponding to the cDNA sequence of SEQ ID NO: 175 from nucleotide 61 to nucleotide 2559, and extending contiguously from a nucleotide sequence corresponding to the 5′ end of said sequence of SEQ ID NO:175 from nucleotide 61 to nucleotide 2559, to a nucleotide sequence corresponding to the 3′ end of said sequence of SEQ ID NO:175 from nucleotide 61 to nucleotide 2559. Also preferably the polynucleotide isolated according to the above process comprises a nucleotide sequence corresponding to the cDNA sequence of SEQ ID NO:175 from nucleotide 904 to nucleotide 2559, and extending contiguously from a nucleotide sequence corresponding to the 5′ end of said sequence of SEQ ID NO:175 from nucleotide 904 to nucleotide 2559, to a nucleotide sequence corresponding to the 3′ end of said sequence of SEQ ID NO:175 from nucleotide 904 to nucleotide 2559.

In other embodiments, the present invention provides a composition comprising a protein, wherein said protein comprises an amino acid sequence selected from the group consisting of:

(a) the amino acid sequence of SEQ ID NO:176;

(b) a fragment of the amino acid sequence of SEQ ID NO:176, the fragment comprising eight contiguous amino acids of SEQ ID NO:176; and

(c) the amino acid sequence encoded by the cDNA insert of clone lv310_{—}7 deposited with the ATCC under accession number 98663;

the protein being substantially free from other mammalian proteins. Preferably such protein comprises the amino acid sequence of SEQ ID NO:176. In further preferred embodiments, the present invention provides a protein comprising a fragment of the amino acid sequence of SEQ ID NO:176 having biological activity, the fragment preferably comprising eight (more preferably twenty, most preferably thirty) contiguous amino acids of SEQ ID NO:176, or a protein comprising a fragment of the amino acid sequence of SEQ ID NO:176 having biological activity, the fragment comprising the amino acid sequence from amino acid 411 to amino acid 420 of SEQ ID NO:176.

In one embodiment, the present invention provides a composition comprising an isolated polynucleotide selected from the group consisting of:

(a) a polynucleotide comprising the nucleotide sequence of SEQ ID NO:177;

(b) a polynucleotide comprising the nucleotide sequence of SEQ ID NO:177 from nucleotide 389 to nucleotide 1330;

(c) a polynucleotide comprising the nucleotide sequence of SEQ ID NO:177 from nucleotide 1286 to nucleotide 1330;

(d) a polynucleotide comprising the nucleotide sequence of the full-length protein coding sequence of clone nq34_{—}12 deposited with the ATCC under accession number 98663;

(e) a polynucleotide encoding the full-length protein encoded by the cDNA insert of clone nq34_{—}12 deposited with the ATCC under accession number 98663;

(f) a polynucleotide comprising the nucleotide sequence of a mature protein coding sequence of clone nq34_{—}12 deposited with the ATCC under accession number 98663;

(g) a polynucleotide encoding a mature protein encoded by the cDNA insert of clone nq34_{—}12 deposited with the ATCC under accession number 98663;

(h) a polynucleotide encoding a protein comprising the amino acid sequence of SEQ ID NO:178;

(i) a polynucleotide encoding a protein comprising a fragment of the amino acid sequence of SEQ ID NO:178 having biological activity, the fragment comprising eight contiguous amino acids of SEQ ID NO:178;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:177.

Preferably, such polynucleotide comprises the nucleotide sequence of SEQ ID NO:177 from nucleotide 389 to nucleotide 1330; the nucleotide sequence of SEQ ID NO:177 from nucleotide 1286 to nucleotide 1330; the nucleotide sequence of the full-length protein coding sequence of clone nq34_{—}12 deposited with the ATCC under accession number 98663; or the nucleotide sequence of a mature protein coding sequence of clone nq34_{—}12 deposited with the ATCC under accession number 98663. In other preferred embodiments, the polynucleotide encodes the full-length or a mature protein encoded by the cDNA insert of clone nq34_{—}12 deposited with the ATCC under accession number 98663. In further preferred embodiments, the present invention provides a polynucleotide encoding a protein comprising a fragment of the amino acid sequence of SEQ ID NO:178 having biological activity, the fragment preferably comprising eight (more preferably twenty, most preferably thirty) contiguous amino acids of SEQ ID NO:178, or a polynucleotide encoding a protein comprising a fragment of the amino acid sequence of SEQ ID NO:178 having biological activity, the fragment comprising the amino acid sequence from amino acid 152 to amino acid 161 of SEQ ID NO:178.

Other embodiments provide the gene corresponding to the cDNA sequence of SEQ ID NO:177.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:177, but excluding the poly(A) tail at the 3′ end of SEQ ID NO:177; and

(ab) the nucleotide sequence of the cDNA insert of clone nq34_{—}12 deposited with the ATCC under accession number 98663;

(ii) hybridizing said probe(s) to human genomic DNA in conditions at least as stringent as 4×SSC at 50 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:177, but excluding the poly(A) tail at the 3′ end of SEQ ID NO:177; and

(bb) the nucleotide sequence of the cDNA insert of clone nq34_{—}12 deposited with the ATCC under accession number 98663;

(ii) hybridizing said primer(s) to human genomic DNA in conditions at least as stringent as 4×SSC at 50 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:177, and extending contiguously from a nucleotide sequence corresponding to the 5′ end of SEQ ID NO:177 to a nucleotide sequence corresponding to the 3′ end of SEQ ID NO:177, but excluding the poly(A) tail at the 3′ end of SEQ ID NO:177. Also preferably the polynucleotide isolated according to the above process comprises a nucleotide sequence corresponding to the cDNA sequence of SEQ ID NO:177 from nucleotide 389 to nucleotide 1330, and extending contiguously from a nucleotide sequence corresponding to the 5 end of said sequence of SEQ ID NO:177 from nucleotide 389 to nucleotide 1330, to a nucleotide sequence corresponding to the 3′ end of said sequence of SEQ ID NO:177 from nucleotide 389 to nucleotide 1330. Also preferably the polynucleotide isolated according to the above process comprises a nucleotide sequence corresponding to the cDNA sequence of SEQ ID NO:177 from nucleotide 1286 to nucleotide 1330, and extending contiguously from a nucleotide sequence corresponding to the 5′ end of said sequence of SEQ ID NO:177 from nucleotide 1286 to nucleotide 1330, to a nucleotide sequence correspondingto the 3′ end of said sequence of SEQ ID NO:177from nucleotide 1286 to nucleotide 1330.

In other embodiments, the present invention provides a composition comprising a protein, wherein said protein comprises an amino acid sequence selected from the group consisting of:

(a) the amino acid sequence of SEQ ID NO:178;

(b) a fragment of the amino acid sequence of SEQ ID NO:178, the fragment comprising eight contiguous amino acids of SEQ ID NO:178; and

(c) the amino acid sequence encoded by the cDNA insert of clone nq34_{—b 12} deposited with the ATCC under accession number 98663;

the protein being substantially free from other mammalian proteins. Preferably such protein comprises the amino acid sequence of SEQ ID NO:178. In further preferred embodiments, the present invention provides a protein comprising a fragment of the amino acid sequence of SEQ ID NO:178 having biological activity, the fragment preferably comprising eight (more preferably twenty, most preferably thirty) contiguous amino acids of SEQ ID NO:178, or a protein comprising a fragment of the amino acid sequence of SEQ ID NO:178 having biological activity, the fragment comprising the amino acid sequence from amino acid 152 to amino acid 161 of SEQ ID NO:178.

In one embodiment, the present invention provides a composition comprising an isolated polynucleotide selected from the group consisting of:

(a) a polynucleotide comprising the nucleotide sequence of SEQ ID NO:179;

(b) a polynucleotide comprising the nucleotide sequence of SEQ ID NO:179 from nucleotide 1026 to nucleotide 1226;

(c) a polynucleotide comprising the nucleotide sequence of SEQ ID NO:179 from nucleotide 1101 to nucleotide 1226;

(d) a polynucleotide comprising the nucleotide sequence of the full-length protein coding sequence of clone pj154_{—}1 deposited with the ATCC under accession number 98663;

(e) a polynucleotide encoding the full-length protein encoded by the cDNA insert of clone pj154_{—}1 deposited with the ATCC under accession number 98663;

(f) a polynucleotide comprising the nucleotide sequence of a mature protein coding sequence of clone pj154_{—}1 deposited with the ATCC under accession number 98663;

(g) a polynucleotide encoding a mature protein encoded by the cDNA insert of clone pj154_{—}1 deposited with the ATCC under accession number 98663;

(h) a polynucleotide encoding a protein comprising the amino acid sequence of SEQ ID NO:180;

(i) a polynucleotide encoding a protein comprising a fragment of the amino acid sequence of SEQ ID NO:180 having biological activity, the fragment comprising eight contiguous amino acids of SEQ ID NO:180;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:179.

Preferably, such polynucleotide comprises the nucleotide sequence of SEQ ID NO:179 from nucleotide 1026 to nucleotide 1226; the nucleotide sequence of SEQ ID NO:179 from nucleotide 1101 to nucleotide 1226; the nucleotide sequence of the full-length protein coding sequence of clone pj154_{—}1 deposited with the ATCC under accession number 98663; or the nucleotide sequence of a mature protein coding sequence of clone pj154_{—}1 deposited with the ATCC under accession number 98663. In other preferred embodiments, the polynucleotide encodes the full-length or a mature protein encoded by the cDNA insert of clone pj154_{—}1 deposited with the ATCC under accession number 98663. In further preferred embodiments, the present invention provides a polynucleotide encoding a protein comprising a fragment of the amino acid sequence of SEQ ID NO:180 having biological activity, the fragment preferably comprising eight (more preferably twenty, most preferably thirty) contiguous amino acids of SEQ ID NO:180, or a polynucleotide encoding a protein comprising a fragment of the amino acid sequence of SEQ ID NO:180 having biological activity, the fragment comprising the amino acid sequence from amino acid 28 to amino acid 37 of SEQ ID NO:180.

Other embodiments provide the gene corresponding to the cDNA sequence of SEQ ID NO:179.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:179, but excluding the poly(A) tail at the 3′ end of SEQ ID NO:179; and

(ab) the nucleotide sequence of the cDNA insert of clone pj154_{—}1 deposited with the ATCC under accession number 98663;

(ii) hybridizing said probe(s) to human genomic DNA in conditions at least as stringent as 4×SSC at 50 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:179, but excluding the poly(A) tail at the 3′ end of SEQ ID NO:179; and

(bb) the nucleotide sequence of the cDNA insert of clone pj154_{—}1 deposited with the ATCC under accession number 98663;

(ii) hybridizing said primer(s) to human genomic DNA in conditions at least as stringent as 4×SSC at 50 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:179, and extending contiguously from a nucleotide sequence corresponding to the 5′ end of SEQ ID NO:179 to a nucleotide sequence corresponding to the 3′ end of SEQ ID NO:179, but excluding the poly(A) tail at the 3′ end of SEQ ID NO:179. Also preferably the polynucleotide isolated according to the above process comprises a nucleotide sequence corresponding to the cDNA sequence of SEQ ID NO:179 from nucleotide 1026 to nucleotide 1226, and extending contiguously from a nucleotide sequence corresponding to the 5′ end of said sequence of SEQ ID NO:179 from nucleotide 1026 to nucleotide 1226, to a nucleotide sequence corresponding to the 3′ end of said sequence of SEQ ID NO:179 from nucleotide 1026 to nucleotide 1226. Also preferably the polynucleotide isolated according to the above process comprises a nucleotide sequence corresponding to the cDNA sequence of SEQ ID NO:179 from nucleotide 1101 to nucleotide 1226, and extending contiguously from a nucleotide sequence corresponding to the 5′ end of said sequence of SEQ ID NO:179 from nucleotide 1101 to nucleotide 1226, to a nucleotide sequence correspondingto the 3′ end of said sequence of SEQIDNO:179 fromnucleotide 1101 to nucleotide 1226.

In other embodiments, the present invention provides a composition comprising a protein, wherein said protein comprises an amino acid sequence selected from the group consisting of:

(a) the amino acid sequence of SEQ ID NO:180;

(b) a fragment of the amino acid sequence of SEQ ID NO:180, the fragment comprising eight contiguous amino acids of SEQ ID NO:180; and

(c) the amino acid sequence encoded by the cDNA insert of clone pj154_{—}1 deposited with the ATCC under accession number 98663;

the protein being substantially free from other mammalian proteins. Preferably such protein comprises the amino acid sequence of SEQ ID NO:180. In further preferred embodiments, the present invention provides a protein comprising a fragment of the amino acid sequence of SEQ ID NO:180 having biological activity, the fragment preferably comprising eight (more preferably twenty, most preferably thirty) contiguous amino acids of SEQ ID NO:180, or a protein comprising a fragment of the amino acid sequence of SEQ ID NO:180 having biological activity, the fragment comprising the amino acid sequence from amino acid 28 to amino acid 37 of SEQ ID NO:180.

In one embodiment, the present invention provides a composition comprising an isolated polynucleotide selected from the group consisting of:

(a) a polynucleotide comprising the nucleotide sequence of SEQ ID NO:181;

(b) a polynucleotide comprising the nucleotide sequence of SEQ ID NO:181 from nucleotide 478 to nucleotide 651;

(c) a polynucleotide comprising the nucleotide sequence of SEQ ID NO:181 from nucleotide 562 to nucleotide 651;

(d) a polynucleotide comprising the nucleotide sequence of the full-length protein coding sequence of clone pk147_{—}1 deposited with the ATCC under accession number 98663;

(e) a polynucleotide encoding the full-length protein encoded by the cDNA insert of clone pk147_{—}1 deposited with the ATCC under accession number 98663;

(f) a polynucleotide comprising the nucleotide sequence of a mature protein coding sequence of clone pk147_{—}1 deposited with the ATCC under accession number 98663;

(g) a polynucleotide encoding a mature protein encoded by the cDNA insert of clone pk147_{—}1 deposited with the ATCC under accession number 98663;

(h) a polynucleotide encoding a protein comprising the amino acid sequence of SEQ ID NO:182;

(i) a polynucleotide encoding a protein comprising a fragment of the amino acid sequence of SEQ ID NO:182 having biological activity, the fragment comprising eight contiguous amino acids of SEQ ID NO:182;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:181.

Preferably, such polynucleotide comprises the nucleotide sequence of SEQ ID NO:181 fromnucleotide 478 to nucleotide 651; the nucleotide sequence of SEQ ID NO:181 from nucleotide 562 to nucleotide 651; the nucleotide sequence of the full-length protein coding sequence of clone pk147_{—}1 deposited with the ATCC under accession number 98663; or the nucleotide sequence of a mature protein coding sequence of clone pk147_{—}1 deposited with the ATCC under accession number 98663. In other preferred embodiments, the polynucleotide encodes the full-length or a mature protein encoded by the cDNA insert of clone pk147_{—}1 deposited with the ATCC under accession number 98663. In further preferred embodiments, the present invention provides a polynucleotide encoding a protein comprising a fragment of the amino acid sequence of SEQ ID NO:182 having biological activity, the fragment preferably comprising eight (more preferably twenty, most preferably thirty) contiguous amino acids of SEQ ID NO:182, or a polynucleotide encoding a protein comprising a fragment of the amino acid sequence of SEQ ID NO:182 having biological activity, the fragment comprising the amino acid sequence from amino acid 24 to amino acid 33 of SEQ ID NO:182.

Other embodiments provide the gene corresponding to the cDNA sequence of SEQ ID NO:181.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:181,but excluding the poly(A) tail at the 3′ end of SEQ ID NO:181; and

(ab) the nucleotide sequence of the cDNA insert of clone pk147_{—}1 deposited with the ATCC under accession number 98663;

(ii) hybridizing said probe(s) to human genomic DNA in conditions at least as stringent as 4×SSC at 50 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:181, but excluding the poly(A) tail at the 3′ end of SEQ ID NO:181; and

(bb) the nucleotide sequence of the cDNA insert of clone pk147_{—}1 deposited with the ATCC under accession number 98663;

(ii) hybridizing said primer(s) to human genomic DNA in conditions at least as stringent as 4×SSC at 50 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:181, and extending contiguously from a nucleotide sequence corresponding to the 5′ end of SEQ ID NO:181 to a nucleotide sequence corresponding to the 3′ end of SEQ ID NO:181, but excluding the poly(A) tail at the 3′ end of SEQ ID NO:181. Also preferably the polynucleotide isolated according to the above process comprises a nucleotide sequence corresponding to the cDNA sequence of SEQ ID NO:181 from nucleotide 478 to nucleotide 651, and extending contiguously from a nucleotide sequence corresponding to the 5′ end of said sequence of SEQ ID NO:181 from nucleotide 478 to nucleotide 651, to a nucleotide sequence corresponding to the 3′ end of said sequence of SEQ ID NO:181 from nucleotide 478 to nucleotide 651. Also preferably the polynucleotide isolated according to the above process comprises a nucleotide sequence corresponding to the cDNA sequence of SEQ ID NO:181 from nucleotide 562 to nucleotide 651, and extending contiguously from a nucleotide sequence corresponding to the 5′ end of said sequence of SEQ ID NO:181 from nucleotide 562 to nucleotide 651, to a nucleotide sequence corresponding to the 3′ end of said sequence of SEQ ID NO:181 from nucleotide 562 to nucleotide 651.

In other embodiments, the present invention provides a composition comprising a protein, wherein said protein comprises an amino acid sequence selected from the group consisting of:

(a) the amino acid sequence of SEQ ID NO:182;

(b) a fragment of the amino acid sequence of SEQ ID NO:182, the fragment comprising eight contiguous amino acids of SEQ ID NO:182; and

(c) the armino acid sequence encoded by the cDNA insert of clone pk147_{—}1 deposited with the ATCC under accession number 98663;

the protein being substantially free from other mammalian proteins. Preferably such protein comprises the amino acid sequence of SEQ ID NO:182. In further preferred embodiments, the present invention provides a protein comprising a fragment of the amino acid sequence of SEQ ID NO:182 having biological activity, the fragment preferably comprising eight (more preferably twenty, most preferably thirty) contiguous amino acids of SEQ ID NO:182, or a protein comprising a fragment of the amino acid sequence of SEQ ID NO:182 having biological activity, the fragment comprising the amino acid sequence from amino acid 24 to amino acid 33 of SEQ ID NO:182.

In one embodiment, the present invention provides a composition comprising an isolated polynucleotide selected from the group consisting of:

(a) a polynucleotide comprising the nucleotide sequence of SEQ ID NO:183;

(b) a polynucleotide comprising the nucleotide sequence of SEQ ID NO:183 from nucleotide 1129 to nucleotide 1896;

(c) a polynucleotide comprising the nucleotide sequence of SEQ ID NO:183 from nucleotide 1189 to nucleotide 1896;

(d) a polynucleotide comprising the nucleotide sequence of the full-length protein coding sequence of clone pt127_{—}1 deposited with the ATCC under accession number 98663;

(e) a polynucleotide encoding the full-length protein encoded by the cDNA insert of clone pt127_{—}1 deposited with the ATCC under accession number 98663;

(f) a polynucleotide comprising the nucleotide sequence of a mature protein coding sequence of clone pt127_{—}1 deposited with the ATCC under accession number 98663;

(g) a polynucleotide encoding a mature protein encoded by the cDNA insert of clone pt127_{—}1 deposited with the ATCC under accession number 98663;

(h) a polynucleotide encoding a protein comprising the amino acid sequence of SEQ ID NO:184;

(i) a polynucleotide encoding a protein comprising a fragment of the amino acid sequence of SEQ ID NO:184 having biological activity, the fragment comprising eight contiguous amino acids of SEQ ID NO:184;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:183.

Preferably, such polynucleotide comprises the nucleotide sequence of SEQ ID NO:183 from nucleotide 1129 to nucleotide 1896; the nucleotide sequence of SEQ ID NO:183 from nucleotide 1189 to nucleotide 1896; the nucleotide sequence of the full-length protein coding sequence of clone pt127_{—}1 deposited with the ATCC under accession number 98663; or the nucleotide sequence of a mature protein coding sequence of clone pt127_{—}1 deposited with the ATCC under accession number 98663. In other preferred embodiments, the polynucleotide encodes the full-length or a mature protein encoded by the cDNA insert of clone pt127_{—}1 deposited with the ATCC under accession number 98663. In further preferred embodiments, the present invention provides a polynucleotide encoding a protein comprising a fragment of the amino acid sequence of SEQ ID NO:184 having biological activity, the fragment preferably comprising eight (more preferably twenty, most preferably thirty) contiguous amino acids of SEQ ID NO:184, or a polynucleotide encoding a protein comprising a fragment of the amino acid sequence of SEQ ID NO:184 having biological activity, the fragment comprising the amino acid sequence from amino acid 123 to amino acid 132 of SEQ ID NO:184.

Other embodiments provide the gene corresponding to the cDNA sequence of SEQ ID NO:183.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ ID NO:183, but excluding the poly(A) tail at the 3′ end of SEQ ID NO:183; and

(ab) the nucleotide sequence of the cDNA insert of clone pt127_{—}1 deposited with the ATCC under accession number 98663;

(ii) hybridizing said probe(s) to human genomic DNA in conditions at least as stringent as 4×SSC at 50 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO: 183, but excluding the poly(A) tail at the 3′ end of SEQ ID NO:183; and

(bb) the nucleotide sequence of the cDNA insert of clone pt127_{—}1 deposited with the ATCC under accession number 98663;

(ii) hybridizing said primer(s) to human genomic DNA in conditions at least as stringent as 4×SSC at 50 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:183, and extending contiguously from a nucleotide sequence corresponding to the 5′ end of SEQ ID NO:183 to a nucleotide sequence corresponding to the 3′ end of SEQ ID NO:183, but excluding the poly(A) tail at the 3′ end of SEQ ID NO:183. Also preferably the polynucleotide isolated according to the above process comprises a nucleotide sequence corresponding to the cDNA sequence of SEQ ID NO:183 from nucleotide 1129 to nucleotide 1896, and extending contiguously from a nucleotide sequence corresponding to the 5′ end of said sequence of SEQ ID NO:183 from nucleotide 1129 to nucleotide 1896, to a nucleotide sequence corresponding to the 3′ end of said sequence of SEQ ID NO:183 from nucleotide 1129 to nucleotide 1896. Also preferably the polynucleotide isolated according to the above process comprises a nucleotide sequence corresponding to the cDNA sequence of SEQ ID NO:183 from nucleotide 1189 to nucleotide 1896, and extending contiguously from a nucleotide sequence corresponding to the 5′ end of said sequence of SEQ ID NO:183 from nucleotide 1189 to nucleotide 1896, to a nucleotide sequence corresponding to the 3′ end of said sequence of SEQ ID NO:183 from nucleotide 1189 to nucleotide 1896.

In other embodiments, the present invention provides a composition comprising a protein, wherein said protein comprises an amino acid sequence selected from the group consisting of:

(a) the amino acid sequence of SEQ ID NO:184;

(b) a fragment of the amino acid sequence of SEQ ID NO:184, the fragment comprising eight contiguous amino acids of SEQ ID NO:184; and

(c) the amino acid sequence encoded by the cDNA insert of clone pt127_{—}1 deposited with the ATCC under accession number 98663;

the protein being substantially free from other mammalian proteins. Preferably such protein comprises the amino acid sequence of SEQ ID NO:184. In further preferred embodiments, the present invention provides a protein comprising a fragment of the amino acid sequence of SEQ ID NO:184 having biological activity, the fragment preferably comprising eight (more preferably twenty, most preferably thirty) contiguous amino acids of SEQ ID NO:184, or a protein comprising a fragment of the amino acid sequence of SEQ ID NO:184 having biological activity, the fragment comprising the amino acid sequence from amino acid 123 to amino acid 132 of SEQ ID NO:184.

In one embodiment, the present invention provides a composition comprising an isolated polynucleotide selected from the group consisting of:

(a) a polynucleotide comprising the nucleotide sequence of SEQ ID NO:185;

(b) a polynucleotide comprising the nucleotide sequence of SEQ ID NO:185 from nucleotide 172 to nucleotide 1041;

(c) a polynucleotide comprising the nucleotide sequence of SEQ ID NO:185 from nucleotide 295 to nucleotide 1041;

(d) a polynucleotide comprising the nucleotide sequence of the full-length protein coding sequence of clone qo115_{—}13 deposited with the ATCC under accession number 98663;

(e) a polynucleotide encoding the full-length protein encoded by the cDNA insert of done qo115_{—}13 deposited with the ATCC under accession number 98663;

(f) a polynucleotide comprising the nucleotide sequence of a mature protein coding sequence of clone qo115_{—}13 deposited with the ATCC under accession number 98663;

(g) a polynucleotide encoding a mature protein encoded by the cDNA insert of clone qo115_{—}13 deposited with the ATCC under accession number 98663;

(h) a polynucleotide encoding a protein comprising the amino acid sequence of SEQ ID NO:186;

(i) a polynucleotide encoding a protein comprising a fragment of the amino acid sequence of SEQ ID NO:186 having biological activity, the fragment comprising eight contiguous amino acids of SEQ ID NO:186;

(j) a polynucleotide which is an allelic variant of a polynucleotide of (a)-(g) above;

(k) a polynucleotide which encodes a species homologue of the protein of (h) or (i) above;

(l) a polynucleotide that hybridizes under stringent conditions to any one of the polynucleotides specified in (a)-(i); and

(m) a polynucleotide that hybridizes under stringent conditions to any one of the polynucleotides specified in (a)-(i) and that has a length that is at least 25% of the length of SEQ ID NO:185.

Preferably, such polynucleotide comprises the nucleotide sequence of SEQ ID NO:185 from nucleotide 172 to nucleotide 1041; the nucleotide sequence of SEQ ID NO:185 fromnucleotide295 tonucleotide 1041; the nucleotide sequence of the full-length protein coding sequence of clone qo115_{—}13 deposited with the ATCC under accession number 98663; or the nucleotide sequence of a mature protein coding sequence of clone qo115_{—}13 deposited with the ATCC under accession number 98663. In other preferred embodiments, the polynucleotide encodes the full-length or a mature protein encoded by the cDNA insert of clone qo115_{—}13 deposited with the ATCC under accession number 98663. In further preferred embodiments, the present invention provides a polynucleotide encoding a protein comprising a fragment of the amino acid sequence of SEQ ID NO:186 having biological activity, the fragment preferably comprising eight (more preferably twenty, most preferably thirty) contiguous amino acids of SEQ ID NO:186, or a polynucleotide encoding a protein comprising a fragment of the amino acid sequence of SEQ ID NO:186 having biological activity, the fragment comprising the amino acid sequence from amino acid 140 to amino acid 149 of SEQ ID NO:186.

Other embodiments provide the gene corresponding to the cDNA sequence of SEQ ID NO:185.

Further embodiments of the invention provide isolated polynucleotides produced according to a process selected from the group consisting of:

(a) a process comprising the steps of:

(i) preparing one or more polynucleotide probes that hybridize in 6×SSC at 65 degrees C to a nucleotide sequence selected from the group consisting of:

(aa) SEQ IDNO:185, but excluding the poly(A) tail atthe 3′ end of SEQ ID NO:185; and

(ab) the nucleotide sequence of the cDNA insert of clone qo115_{—}13 deposited with the ATCC under accession number 98663;

(ii) hybridizing said probe(s) to human genomic DNA in conditions at least as stringent as 4×SSC at 50 degrees C; and

(iii) isolating the DNA polynucleotides detected with the probe(s); and

(b) a process comprising the steps of:

(i) preparing one or more polynucleotide primers that hybridize in 6×SSC at 65 degrees C to a nucleotide sequence selected from the group consisting of:

(ba) SEQ ID NO:185, but excluding the poly(A) tail at the 3′ end of SEQ ID NO:185; and

(bb) the nucleotide sequence of the cDNA insert of clone qo115_{—}13 deposited with the ATCC under accession number 98663;

(ii) hybridizing said primer(s) to human genomic DNA in conditions at least as stringent as 4×SSC at 50 degrees C;

(iii) amplifying human DNA sequences; and

(iv) isolating the polynucleotide products of step (b)(iii).

Preferably the polynucleotide isolated according to the above process comprises a nucleotide sequence corresponding to the cDNA sequence of SEQ ID NO:185, and extending contiguously from a nucleotide sequence corresponding to the 5′ end of SEQ ID NO:185 to a nucleotide sequence corresponding to the 3′ end of SEQ ID NO:185, but excluding the poly(A) tail at the 3′ end of SEQ ID NO:185. Also preferably the polynucleotide isolated according to the above process comprises a nucleotide sequence corresponding to the cDNA sequence of SEQ ID NO:185 from nucleotide 172 to nucleotide 1041, and extending contiguously from a nucleotide sequence corresponding to the 5′ end of said sequence of SEQ ID NO:185 from nucleotide 172 to nucleotide 1041, to a nucleotide sequence corresponding to the 3′ end of said sequence of SEQ ID NO:185 from nucleotide 172 to nucleotide 1041. Also preferably the polynucleotide isolated according to the above process comprises a nucleotide sequence corresponding to the cDNA sequence of SEQ ID NO:185 from nucleotide 295 to nucleotide 1041, and extending contiguously from a nucleotide sequence corresponding to the 5′ end of said sequence of SEQ ID NO:185 from nucleotide 295 to nucleotide 1041, to a nucleotide sequence corresponding to the 3′ end of said sequence of SEQ ID NO:185 from nucleotide 295 to nucleotide 1041.

In other embodiments, the present invention provides a composition comprising a protein, wherein said protein comprises an amino acid sequence selected from the group consisting of:

(a) the amino acid sequence of SEQ ID NO:186;

(b) a fragment of the amino acid sequence of SEQ ID NO:186, the fragment comprising eight contiguous amino acids of SEQ ID NO:186; and

(c) the amino acid sequence encoded by the cDNA insert of clone qo115_{—}13 deposited with the ATCC under accession number 98663;

the protein being substantially free from other mammalian proteins. Preferably such protein comprises the amino acid sequence of SEQ ID NO:186. In further preferred embodiments, the present invention provides a protein comprising a fragment of the amino acid sequence of SEQ ID NO:186 having biological activity, the fragment preferably comprising eight (more preferably twenty, most preferably thirty) contiguous amino acids of SEQ ID NO:186, or a protein comprising a fragment of the amino acid sequence of SEQ ID NO:186 having biological activity, the fragment comprising the amino acid sequence from amino acid 140 to amino acid 149 of SEQ ID NO:186.

In certain preferred embodiments, the polynucleotide is operably linked to an expression control sequence. The invention also provides a host cell, including bacterial, yeast, insect and mammalian cells, transformed with such polynucleotide compositions. Also provided by the present invention are organisms that have enhanced, reduced, or modified expression of the gene(s) corresponding to the polynucleotide sequences disclosed herein.

Processes are also provided for producing a protein, which comprise:

(a) growing a culture of the host cell transformed with such polynucleotide compositions in a suitable culture medium; and

(b) purifying the protein from the culture.

The protein produced according to such methods is also provided by the present invention.

Protein compositions of the present invention may further comprise a pharmaceutically acceptable carrier. Compositions comprising an antibody which specifically reacts with such protein are also provided by the present invention.

Methods are also provided for preventing, treating or ameliorating a medical condition which comprises administering to a mammalian subject a therapeutically effective amount of a composition comprising a protein of the present invention and a pharmaceutically acceptable carrier.

### BRIEF DESCRIPTION OF THE DRAWINGS

FIGS. 1A and 1B are schematic representations of the pED6 and pNOTs vectors, respectively, used for deposit of clones disclosed herein.

### DETAILED DESCRIPTION

Isolated Proteins and Polynucleotides

Nucleotide and amino acid sequences, as presently determined, are reported below for each clone and protein disclosed in the present application. The nucleotide sequence of each clone can readily be determined by sequencing of the deposited clone in accordance with known methods. The predicted amino acid sequence (both full-length and mature forms) can then be determined from such nucleotide sequence. The amino acid sequence of the protein encoded by a particular clone can also be determined by expression of the clone in a suitable host cell, collecting the protein and determining its sequence. For each disclosed protein applicants have identified what they have determined to be the reading frame best identifiable with sequence information available at the time of filing.

As used herein a “secreted” protein is one which, when expressed in a suitable host cell, is transported across or through a membrane, including transport as a result of signal sequences in its amino acid sequence. “Secreted” proteins include without limitation proteins secreted wholly (e.g., soluble proteins) or partially (e.g., receptors) from the cell in which they are expressed. “Secreted” proteins also include without limitation proteins which are transported across the membrane of the endoplasmic reticulum.

Clone “bd306_{—}7”

A polynucleotide of the present invention has been identified as clone “bd306_{—}7”. bd306_{—}7 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bd306_{—}7 is a full-length clone, including the entire coding sequence of a secreted protein (also referred to herein as “bd306_{—}7 protein”).

The nucleotide sequence of bd306_{—}7 as presently determined is reported in SEQ ID NO: 1, and includes a poly(A) tail. What applicants presently believe to be the proper reading frame and the predicted amino acid sequence of the bd306_{—}7 protein corresponding to the foregoing nucleotide sequence is reported in SEQ ID NO:2. Amino acids 11 to 23 are a predicted leader/signal sequence, with the predicted mature amino acid sequence beginning at amino acid 24. Due to the hydrophobic nature of the predicted leader/signal sequence, it is likely to act as a transmembrane domain should the predicted leader/signal sequence not be separated from the remainder of the bd306_{—}7 protein.

The EcoRI/NotI restriction fragment obtainable from the deposit containing clone bd306_{—}7 should be approximately 3700 bp.

The nucleotide sequence disclosed herein for bd306_{—}7 was searched against the GenBank and GeneSeq nucleotide sequence databases using BLASTN/BLASTX and FASTA search protocols. bd306_{—}7 demonstrated at least some similarity with sequences identified as AA027096 (zk04d03.s1 Soares pregnant uterus NbHPU Homo sapiens cDNA clone 469541 3′), AA027135 (zk04d03.r1 Soares pregnant uterus NbHPU Homo sapiens cDNA clone 469541 5′), AA166312 (ms42g11.r1 Life Tech mouse embryo 135dpc 10666014 Mus musculus cDNA clone 6142765′ similar to TR E238793 E238793 DUALIN, AA535890 (nf94a03.s1 NCI_CGAP_Co3 Homo sapiens cDNA clone IMAGE:927532), H14467 (yl25g07.r1 Homo sapiens cDNA clone 159324 5′ similar to contains HGR repetitive element), T21281 (Human gene signature HUMGS02637), T61016 (Total DNA sequence from cosmid clones LP(2)127 and LP(2)128), U47621 (Human nucleolar autoantigen No55 mRNA, complete cds), W51808 (zc48g04.r1 Soares senescent fibroblasts NbHSF Homo sapiens cDNA clone 325590 5′ similar to PIR:S20742 S20742 synaptonemal complex protein Sc65-rat; contains Alu repetitive element; mRNA sequence), and X97607 (G.gallus mRNA for cartilage associated protein). The predicted amino acid sequence disclosed herein for bd306_{—}7 was searched against the GenPept and GeneSeq amino acid sequence databases using the BLASTX search protocol. The predicted bd306_{—}7 protein demonstrated at least some similarity to sequences identified as R95913 (Neural thread protein [Homo sapiens]), U47621 (nucleolar autoantigen No55 [Homo sapiens]), and X97607 (cartilage associated protein [Gallus gallus]). Two regions of bd306_{—}7 protein (amino acids 148_{—}217 and 298_{—}367 of SEQ ID NO:2) align with the same region, amino acids 145_{—}214, of cartilage associated protein. The homology between bd306_{—}7 protein and nucleolar autoantigen No55 is also good, but in this case it appears that bd306_{—}7 amino acids 148_{—}189 is similar to two regions of No55 (amino acids 145_{—}186 and 296_{—}337), and bd306_{—}7 amino acids 298_{—}367 are also similar to nearby the same two regions of No55 (amino acids 145_{—}214 and 296_{—}365). This implies that two regions in bd306_{—}7 (roughly 148_{—}189 and 298_{—}367) are similar to each other, and one copy of this region is found in cartilage associated protein, but both are present in No55. Cartilage associated protein is reported tobe localized to the extracelluar matrix {J. Cell Sci 1997 110(Pt 12):1351_{—}1359}, while No55 is found in the granular component of the nucleolus {Mol Biol Cell 1996 7(7):1015_{—}1024}. Based upon sequence similarity, bd306_{—}7 proteins and each similar protein or peptide may share at least some activity. The nucleotide sequence of bd306_{—}7 also indicates that it may contain an Alu repetitive element.

bd306_{—}7 protein was expressed in a COS cell expression system, and an expressed protein band of approximately 52 kDa was detected in membrane fractions using SDS polyacrylamide gel electrophoresis.

Clone “fj283_{—}11” and Clone “fj283_{—}6”

Polynucleotides of the present invention have been identified as clone “fj283_{—}11” and clone “fj283_{—}6”. fj283_{—}11 and fj283_{—}6 were isolated from a human adult lung carcinoma cDNA library using methods which are selective for cDNAs encoding secreted proteins (see U.S. Pat. No. 5,536,637), or were identified as encoding a secreted or transmembrane protein on the basis of computer analysis of the amino acid sequence of the encoded protein. fj283_{—}11 and fj283_{—}6 are full-length clones, including the entire coding sequence of a secreted protein (also referred to herein as “fj283 protein”).

The nucleotide sequence of fj283_{—}11 as presently determined is reported in SEQ ID NO:3, and includes a poly(A) tail. The nucleotide sequence of fj283_{—}6 as presently determined is reported in SEQ ID NO:198, and includes a poly(A) tail. Although cDNA clones fj283_{—}11 and fj283_{—}6 have different nucleotide sequences, perhaps as a result of alternative splicing of a common primary MRNA transcript (particularly between nucleotide 402 and nucleotide 618 of SEQ ID NO:198), these clones are predicted to encode the same protein. What applicants presently believe to be the proper reading frame and the predicted amino acid sequence of the fj283 protein corresponding to the foregoing nucleotide sequences is reported in SEQ ID NO:4. Amino acids 8 to 20 of SEQ ID NO:4 are a predicted leader/signal sequence, with the predicted mature amino acid sequence beginning at amino acid 21. Due to the hydrophobic nature of the predicted leader/signal sequence, it is likely to act as a transmembrane domain should the predicted leader/signal sequence not be separated from the remainder of the fj283 protein.

The EcoRI/NotI restriction fragment obtainable from the deposit containing clone fj283_{—}11 should be approximately 3350 bp. The EcoRI/NotI restriction fragment obtainable from the deposit containing clone fj283_{—}6 should be approximately 2700 bp.

The nucleotide sequences disclosed herein for fj283_{—}11 and fj283_{—}6 were searched against the GenBank and GeneSeq nucleotide sequence databases using BLASTN/BLASTX and FASTA search protocols. fj283_{—}11 and/or fj283_{—}6 demonstrated at least some similarity with sequences identified as AA052962 (zl70c02.s1 Stratagene colon (#937204) Homo sapiens cDNA clone 509954 3′ similar to gb D14531 60S RIBOSOMAL PROTEIN L9 (HUMAN)), AA080949 (zn04d12.r1 Stratagene hNT), AA160948 (zq40e12.r1 Stratagene hNT neuron (#937233) Homo sapiens cDNA clone 632206 5′), AA195089 (zr34c02.r1 Soares NhHMPu S1 Homo sapiens cDNA clone 665282 5′, mRNA sequence), AA258887 (zs32b02.r1 NCI_CGAP_GCB1 Homo sapiens cDNA clone IMAGE:686859 5′), H97993 (yw06e03.s1 Homo sapiens cDNA clone 251452 3′), R19768 (yg40g06.r1 Homo sapiens cDNA clone 34951 5′), U09953 (Human ribosomal protein L9 mRNA, complete cds), Z73639 (Human DNA sequence from cosmid V389H8 on chromosome X; Contains MRNA near btk gene involved in a-gamma-globulinemia, ESTs, STS), and Z73901 (Human DNA sequence from cosmid V389H8, between markers DXS366 and DXS87 on chromosome X contains pseudogene, mRNA near btk gene involved in a-gamma-globulinemia, ESTs, STSs). The predicted amino acid sequence disclosed herein for the fj283 protein was searched against the GenPept and GeneSeq amino acid sequence databases using the BLASTX search protocol. The predicted fj283 protein demonstrated at least some similarity to sequences identified as AB011084 (KIAA0512 protein [Homo sapiens]) and U09953 (ribosomal protein L9 [Homo sapiens]). Based upon sequence similarity, fj283 proteins and each similar protein or peptide may share at least some activity. Profile hidden markov model analysis has revealed the presence of an Armadillo/beta-catenin-like domain within the predicted fj283 protein sequence. The armadillo multigene family comprises many proteins widely differing in sizes and functions which have in common a variable number of tandemly repeated arm sequences of about 42 amino acids in length. Many, but not all, armadillo-repeat-containing proteins are nuclear in localization. The predicted fj283 protein does not appear to be of the nuclear variety, but rather appears to be an extracellular protein.

Clone “fk317_{—}3”

A polynucleotide of the present invention has been identified as clone “fk317_{—}3”. fk317_{—}3 was isolated from a human adult kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fk317_{—}3 is a full-length clone, including the entire coding sequence of a secreted protein (also referred to herein as “fk317_{—}3 protein”). The nucleotide sequence of fk317_{—}3 as presently determined is reported in SEQ ID NO:5, and includes a poly(A) tail. What applicants presently believe to be the proper reading frame and the predicted amino acid sequence of the fk317_{—}3 protein corresponding to the foregoing nucleotide sequence is reported in SEQ ID NO:6.

The EcoRI/NotI restriction fragment obtainable fromthe deposit containingclone fk317_{—}3 should be approximately 1400 bp.

The nucleotide sequence disclosed herein for fk317_{—}3 was searched against the GenBank and GeneSeq nucleotide sequence databases using BLASTN/BLASTX and FASTA search protocols. fk317_{—}3 demonstrated at least some similarity with sequences identified as AA568588 (nm21b11.s1 NCI_CGAP_Co10 Homo sapiens cDNA clone IMAGE:1060797), AC002326 (Genomic sequence from Human 6, complete sequence), H48562 (yq78g07.s1 Homo sapiens cDNA clone 201948 3′ similar to contains Alu repetitive element; contains MER30 repetitive element), T67164 (Human alpha-N-acetylglucosaminidase gene), and Z46941 (H.sapiens DNA for alu repeats). The predicted amino acid sequence disclosed herein for fk317_{—}3 was searched against the GenPept and GeneSeq amino acid sequence databases using the BLASTX search protocol. The predicted fk317_{—}3 protein demonstrated at least some similarity to sequences identified as X55777 (put. ORF [Homo sapiens]). Based upon sequence similarity, fk317_{—}3 proteins and each similar protein or peptide may share at least some activity. The TopPredII computer program predicts a potential transmembrane domain within the fk317_{—}3 protein sequence centered around amino acid 42 of SEQ ID NO:6. The nucleotide sequence of fk317_{—}3 indicates that it may contain an Alu repetitive element.

Clone “k213_{—}2x”

A polynucleotide of the present invention has been identified as clone “k213_{—}2x”. Secreted cDNA clones were first isolated from a murine adult bone marrow (stromal cell line FCM-4) cDNA library using methods which are selective for cDNAs encoding secreted proteins (see U.S. Pat. No. 5,536,637), or were identified as encoding a secreted or transmembrane protein on the basis of computer analysis of the amino acid sequence of the encoded protein. These murine cDNAs were then used to isolate k213_{—}2x, a full-length human cDNA clone, including the entire coding sequence of a secreted protein (also referred to herein as “k213_{—}2x protein”).

The nucleotide sequence of k213_{—}2x as presently determined is reported in SEQ ID NO:7, and includes a poly(A) tail. What applicants presently believe to be the proper reading frame and the predicted amino acid sequence of the k213_{—}2x protein corresponding to the foregoing nucleotide sequence is reported in SEQ ID NO:8. Amino acids 26 to 38 are a predicted leader/signal sequence, with the predicted mature amino acid sequence beginning at amino acid 39. Due to the hydrophobic nature of the predicted leader/signal sequence, it is likely to act as a transmembrane domain should the predicted leader/signal sequence not be separated from the remainder of the k213_{—}2x protein.

The EcoRI/NotI restriction fragment obtainable from the deposit containing clone k213_{—}2x should be approximately 1900 bp.

The nucleotide sequence disclosed herein for k213_{—}2x was searched against the GenBank and GeneSeq nucleotide sequence databases using BLASTN/BLASTX and FASTA search protocols. k213_{—}2x demonstrated at least some similarity with sequences identified as AA123852 (mp96e08.r1 Soares 2NbMT Mus musculus cDNA clone 577094 5′), AA362005 (EST71348 T-cell lymphoma Homo sapiens cDNA 5′ end), AA436477 (zv08f05.s1 Soares NhHMPu S1 Homo sapiens cDNA clone 753057 3′), AA436528 (zv08fOb 05.r1 Soares NhHMPu SI Homo sapiens cDNA clone 753057 5′), AA643506 (nq86f04.s1 NCI_CGAP_Co9 Homo sapiens cDNA clone IMAGE:1159231, mRNA sequence), F13485 (H. sapiens partial cDNA sequence; clone c-3dh08), and T19502 (Human gene signature HUMGS00560). Based upon sequence similarity, k213_{—}2x proteins and each similar protein or peptide may share at least some activity.

k213_{—}2x protein was expressed in a COS cell expression system, and an expressed protein band of approximately 6 kDa was detected in membrane fractions using SDS polyacrylamide gel electrophoresis.

Clone “na316_{—}1”

A polynucleotide of the present invention has been identified as clone “na316_{—}1”. na316_{—}1 was isolated from a human adult brain (corpus callosum) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. na316_{—}1 is a full-length clone, including the entire coding sequence of a secreted protein (also referred to herein as “na316_{—}1 protein”). The nucleotide sequence of na316_{—}1 as presently determined is reported in SEQ ID NO:9, and includes a poly(A) tail. What applicants presently believe to be the proper reading frame and the predicted amino acid sequence of the na316_{—}1 protein corresponding to the foregoing nucleotide sequence is reported in SEQ ID NO:10. Amino acids 30 to 42 are a predicted leader/signal sequence, with the predicted mature amino acid sequence beginning at amino acid 43. Due to the hydrophobic nature of the predicted leader/signal sequence, it is likely to act as a transmembrane domain should the predicted leader/signal sequence not be separated from the remainder of the na316_{—}1 protein.

The EcoRI/NotI restriction fragment obtainable from the deposit containing clone na316_{—}1 should be approximately 900 bp.

The nucleotide sequence disclosed herein for na316_{—}1 was searched against the GenBank and GeneSeq nucleotide sequence databases using BLASTN/BLASTX and FASTA search protocols. No hits were found in the database. The TopPredII computer program predicts two potential transmembrane domains within the na316_{—}1 protein sequence, centered around amino acids 31 and 66 of SEQ ID NO:10, respectively.

Clone “nf93_{—20”}

A polynucleotide of the present invention has been identified as clone “nf93_{—}20”. nf93_{—}20 was isolated from a human adult brain (substantia nigra) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. nf93_{—}20 is a full-length clone, including the entire coding sequence of a secreted protein (also referred to herein as “nf93_{—}20 protein”).

The nucleotide sequence of nf93_{—}20 as presently determined is reported in SEQ ID NO:11, and includes a poly(A) tail. What applicants presently believe to be the proper reading frame and the predicted amino acid sequence of the nf93_{—}20 protein corresponding to the foregoing nucleotide sequence is reported in SEQ ID NO: 12. Amino acids 6 to 18 are a predicted leader/signal sequence, with the predicted mature amino acid sequence beginning at amino acid 19. Due to the hydrophobic nature of the predicted leader/signal sequence, it is likely to act as a transmembrane domain should the predicted leader/signal sequence not be separated from the remainder of the nf93_{—}20 protein.

The EcoRI/NotI restriction fragment obtainable from the deposit containing clone nf93_{—}20 should be approximately 2000 bp.

The nucleotide sequence disclosed herein for nf93_{—}20 was searched against the GenBank and GeneSeq nucleotide sequence databases using BLASTN/BLASTX and FASTA search protocols. nf93_{—}20 demonstrated at least some similarity with sequences identified as AA063620 (ze87g07.s1 Soares fetal heart NbHH19W Homo sapiens cDNA clone 366012 3′), AA317410 (EST19337 Retina II Homo sapiens cDNA 5′ end), H29417 (ym60e07.r1 Homo sapiens cDNA clone 526315′), and N41425 (yw93e08.r1 Homo sapiens cDNA clone 259814 5′). Based upon sequence similarity, nf93_{—}20 proteins and each similar protein or peptide may share at least some activity.

nf93_{—}20 protein was expressed in a COS cell expression system, and an expressed protein band of approximately 29 kDa was detected in membrane fractions using SDS polyacrylarnide gel electrophoresis.

Clone “np164_{—}1”

A polynucleotide of the present invention has been identified as clone “np164_{—}1 ”. np164_{—}1 was isolated from a human fetal kidney (293 cell line) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. np164_{—}1 is a full-length clone, including the entire coding sequence of a secreted protein (also referred to herein as “np164_{—}1 protein”).

The nucleotide sequence of np164_{—}1 as presently determined is reported in SEQ ID NO:13, and includes a poly(A) tail. What applicants presently believe to be the proper reading frame and the predicted amino acid sequence of the np164_{—}1 protein corresponding to the foregoing nucleotide sequence is reported in SEQ ID NO:14. Amino acids 348 to 360 are a predicted leader/signal sequence, with the predicted mature amino acid sequence beginning at amino acid 361. Due to the hydrophobic nature of the predicted leader/signal sequence, it is likely to act as a transmembrane domain should the predicted leader/signal sequence notbe separated from the remainder of the np164_{—}1 protein.

The EcoRI/NotI restriction fragment obtainable from the deposit containing clone np164_{—}1 should be approximately 2100 bp.

The nucleotide sequence disclosed herein for np164_{—}1 was searched against the GenBank and GeneSeq nucleotide sequence databases using BLASTN/BLASTX and FASTA search protocols. np164_{—}1 demonstrated at least some similarity with sequences identified as N63143 (yz37c12.s1 Homo sapiens cDNA clone 285238 3′), T19992 (Human gene signature HUMGS01129), Z46676 (Caenorhabditis elegans cosmid C08B11, complete sequence), and Z74910 (S.cerevisiae chromosome XV reading frame ORF YOR002w). The predicted amino acid sequence disclosed herein for np164_{—}1 was searched against the GenPept and GeneSeq amino acid sequence databases using the BLASTX search protocol. The predicted np164_{—}1 protein demonstrated at least some similarity to sequences identified as Z46676 (C08B11.8 [Caenorhabditis elegans]) and Z74910 (ORF YOR002w [Saccharomyces cerevisiae]). Based upon sequence similarity, np164_{—}1 proteins and each similar protein or peptide may share at least some activity. The TopPredlI computer program predicts ten potential transmembrane domains within the np164_{—}1 protein sequence, centered around amino acids 4, 114, 165,229,293,322, 360,386,436, and 465 of SEQ ID NO:14, respectively.

np164_{—}1 protein was expressed in a COS cell expression system, and an expressed protein band of approximately 43 kDa was detected in membrane fractions using SDS polyacrylamide gel electrophoresis.

Clone “pe204_{—}1”

A polynucleotide of the present invention has been identified as clone “pe204_{—}1”. pe204_{—}1 was isolated from a human adult blood (chronic myelogenous leukemia K5) cDNA library using methods which are selective for cDNAs encoding secreted proteins (see U.S. Pat. No.5,536,637), or was identified as encoding a secreted or transmembrane protein on the basis of computer analysis of the amino acid sequence of the encoded protein. pe204_{—}1 is a full-length clone, including the entire coding sequence of a secreted protein (also referred to herein as “pe204_{—}1 protein”).

The nucleotide sequence of pe204_{—}1 as presently determined is reported in SEQ ID NO:15, and includes a poly(A) tail. What applicants presently believe to be the proper reading frame and the predicted amino acid sequence of the pe204_{—}1 protein corresponding to the foregoing nucleotide sequence is reported in SEQ ID NO:16. Amino acids 116 to 128 are a predicted leader/signal sequence, with the predicted mature amino acid sequence beginning at amino acid 129. Due to the hydrophobic nature of the predicted leader/signal sequence, it is likely to act as a transmembrane domain should the predicted leader/signal sequence not be separated from the remainder of the pe204_{—}1 protein.

The EcoRI/NotI restriction fragment obtainable from the deposit containing clone pe204_{—}1 should be approximately 1100 bp.

The nucleotide sequence disclosed herein for pe204_{—}1 was searched against the GenBank and GeneSeq nucleotide sequence databases using BLASTN/BLASTX and FASTA search protocols. pe204_{—}1 demonstrated at least some similarity with sequences identified as AA279961 (zs92h08.s1 NCI_CGAP_GCB1 Homo sapiens cDNA clone 70499] 3′), AA306911 (EST178043 Colon carcinoma (HCC) cell line Homo sapiens cDNA 5′ end), AC002086 (Human PAC clone DJ525N14), AC002094 (Genomic sequence from Human 17, complete sequence), T97749 (ye58c04.s1 Homo sapiens cDNA clone), Z74696 (Human DNA sequence from cosmid 203C2, between markers DXS6791 and DXS8038 on chromosome X contains ESTs), Z80901 (Human DNA sequence from cosmid N119A7 on chromosome 22q12-qter), and Z82245 (Human DNA sequence *** SEQUENCING IN PROGRESS *** from clone 799F10; HTGS phase 1). The predicted amino acid sequence disclosed herein for pe204_{—}1 was searched against the GenPept and GeneSeq amino acid sequence databases using the BLASTX search protocol. The predicted pe204_{—}1 protein demonstrated at least some similarity to sequences identified as K02113 (Gallus gallus vitellogenin [Callus gallus]). Based upon sequence similarity, pe204_{—}1 proteins and each similar protein or peptide may share at least some activity. The TopPredII computer program predicts two additional potential transmembrane domains within the pe204_{—}1 protein sequence, one centered around amino acid 50 and another around amino acid 90 of SEQ ID NO:16.

pe204_{—}1 protein was expressed in a COS cell expression system, and an expressed protein band of approximately 14 kDa was detected in membrane fractions using SDS polyacrylamide gel electrophoresis.

Clone “ya 1_{—}1 ”

A polynucleotide of the present invention has been identified as clone “ya1_{—}1 ”. ya1_{—}1 was isolated from a human adult testes cDNA library and was identified as encoding a secreted or transmembrane protein on the basis of computer analysis of the amino acid sequence of the encoded protein. Ya1_{—}1 is a full-length clone, including the entire coding sequence of a secreted protein (also referred to herein as “ya1_{—}1 protein”).

The nucleotide sequence of ya1_{—}1 as presently determined is reported in SEQ ID NO:17, and includes a poly(A) tail. What applicants presently believe to be the proper reading frame and the predicted amino acid sequence of the ya1_{—}1 protein corresponding to the foregoing nucleotide sequence is reported in SEQ ID NO:18. Amino acids 330 to 342 are a predicted leader/signal sequence, with the predicted mature amino acid sequence beginning at amino acid 343. Due to the hydrophobic nature of the predicted leader/signal sequence, it is likely to act as a transmembrane domain should the predicted leader/signal sequence not be separated from the remainder of the ya1_{—}1 protein.

The EcoRI/NotI restriction fragment obtainable from the deposit containing clone ya1_{—}1 should be approximately 1400 bp.

The nucleotide sequence disclosed herein for yal1_{—}1 was searched against the GenBank and GeneSeq nucleotide sequence databases using BLASTN/BLASTX and FASTA search protocols. Ya1_{—}1 demonstrated at least some similarity with sequences identified as AA431507 (zw76e05.r1 Soares testis NHT Homo sapiens cDNA clone 782144 5′) and F03332 (H. sapiens partial cDNA sequence; clone c-1tg07). Based upon sequence similarity, ya1_{—}1 proteins and each similar protein or peptide may share at least some activity. The TopPredII computer program predicts two potential transmembrane domains within the yalI protein sequence centered around amino acid 156 and around amino acid 332 of SEQ ID NO:18, respectively. The nucleotide sequence of yalJ indicates that it may contain an Alu repetitive element.

ya1_{—}1 protein was expressed in a COS cell expression system, and an expressed protein band of approximately 38 kDa was detected in membrane fractions using SDS polyacrylamide gel electrophoresis.

Clone “yb8_{—}1”

A polynucleotide of the present invention has been identified as clone “yb8_{—}1”. yb8_{—}1 was isolated from a human fetal brain cDNA library and was identified as encoding a secreted or transmembrane protein on the basis of computer analysis of the amino acid sequence of the encoded protein. yb8_{—}1 is a full-length clone, including the entire coding sequence of a secreted protein (also referred to herein as “yb8_{—}1 protein”).

The nucleotide sequence of yb8_{—}1 as presently determined is reported in SEQ ID NO:19, and includes a poly(A) tail. What applicants presently believe to be the proper reading frame and the predicted amino acid sequence of the yb8_{—}1 protein corresponding to the foregoing nucleotide sequence is reported in SEQ ID NO:20. Amino acids 69 to 81 are a predicted leader/signal sequence, with the predicted mature amino acid sequence beginning at amino acid 82. Due to the hydrophobic nature of the predicted leader/signal sequence, it is likely to act as a transmembrane domain should the predicted leader/signal sequence not be separated from the remainder of the yb8_{—}1 protein.

The EcoRI/NotI restriction fragment obtainable from the deposit containing clone yb8_{—}1 should be approximately 1800 bp.

The nucleotide sequence disclosed herein for yb8_{—}1 was searched against the GenBank and GeneSeq nucleotide sequence databases using BLASTN/BLASTX and FASTA search protocols. yb8_{—}1 demonstrated at least some similarity with sequences identified as AA418057 (zv97a06.r1 Soares iNhHMPu Si Homo sapiens cDNA clone 767698 5′ similar to TR:G1143719 G1143719 RS-REX-B), L10334 (Homo sapiens neuroendocrine-specific protein B (NSP) MRNA, complete cds), U17603 (Rattus norvegicus rS-Rex-s mRNA, complete cds), and W19986 (zb38e09.r1 Soares parathyroid tumor NbHPA Homo sapiens cDNA clone 305896 5′ , mRNA sequence). The predicted amino acid sequence disclosed herein for yb8_{—}1 was searched against the GenPept and GeneSeq amino acid sequence databases using the BLASTX search protocol. The predicted yb8_{—}1 protein demonstrated at least some similarity to sequences identified as L10334 (neuroendocrine-specific proteins B and C [Homo sapiens]) and U17603 (rS-Rex-s [Rattus norvegicus]). Based upon sequence similarity, yb8_{—}1 proteins and each similar protein or peptide may share at least some activity. The predicted yb8_{—}1 protein shows significant (60% identity) amino acid similarity to the neuro-endocrine specific protein (NSP) family of proteins. Roebroek et al. (1993, J. Biol Chem. 268: 13439, which is incoporated by reference herein) report observing three transcripts from this gene family: NSP-A (3.4 kb), -B (2.3 kb), and -C (1.8 kb); they encode proteins of 776, 356, and 208 amino acids, respectively. Roebroek et al. also observe that these three transcripts are identical at the 3′ end and only differ over a short portion near their 5′ ends, and are thus possible splice variants. NSP-A and NSP-C were found in neural and endocrine tissues while NSP-B was found only in a lung carcinoma cell line (Roebrek et al. state that NSP-B is “aberrant” suggesting that it might be an artifact). The C-teminal portions of the protein sequences from all three transcripts are identical. The predicted yb8_{—}1 protein shows strong amino acid similarity within this region and is about as long as NSP-C. Thus the predicted yb8_{—}1 protein appears to be related to NSP-C. The TopPredII computer program predicts two potential transmembrane domains within the yb8_{—}1 protein sequence, centered around amino acids 82 and 174 of SEQ ID NO:20, respectively. yb8_{—}1 protein was expressed in a COS cell expression system, and an expressed protein band of approximately 25 kDa was detected in membrane fractions and in comditioned medium using SDS polyacrylamide gel electrophoresis.

Clone “am856_{—}3”

A polynucleotide of the present invention has been identified as clone “am856_{—}3”. am856_{—}3 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. am856_{—}3 is a full-length clone, including the entire coding sequence of a secreted protein (also referred to herein as “am856_{—}3 protein”).

The nucleotide sequence of am856_{—}3 as presently determined is reported in SEQ ID NO:21, and includes a poly(A) tail. What applicants presently believe to be the proper reading frame and the predicted amino acid sequence of the am856_{—}3 protein corresponding to the foregoing nucleotide sequence is reported in SEQ ID NO:22. Amino acids 23 to 35 are a predicted leader/signal sequence, with the predicted mature amino acid sequence beginning at amino acid 36. Due to the hydrophobic nature of the predicted leader/signal sequence, it is likely to act as a transmembrane domain should the predicted leader/signal sequence not be separated from the remainder of the am856_{—}3 protein. The amino acid sequence of another protein that could be encoded by basepairs 214 to 369 of SEQ ID NO:21 is reported in SEQ ID NO:274.

The EcoRI/NotI restriction fragment obtainable from the deposit containing clone am856_{—}3 should be approximately 2100 bp.

The nucleotide sequence disclosed herein for am856_{—}3 was searched against the GenBank and GeneSeq nucleotide sequence databases using BLASTN/BLASTX and FASTA search protocols. am856_{—}3 demonstrated at least some similarity with sequences identified as M26434 (Human hypoxanthine phosphoribosyltransferase (HPRT) gene, complete cds), N71723 (yw52b09.r1 Homo sapiens cDNA clone 255833 5′ similar to gb |M87920| HUMALNE652 Human carcinoma cell-derived Alu RNA transcript, (rRNA);

gb X77738_mal BAND 3 ANION TRANSPORT PROTEIN), U41196 (Human (TTTC)5 short tandem repeat polymorphism UM69, D17S1339), and X89398 (H.sapiens ung gene for uracil DNA-glycosylase). Based upon sequence similarity, am856_{—}3 proteins and each similar protein or peptide may share at least some activity. The TopPredII computer program predicts the amino-terminal half of the am856_{—}3 protein sequence to be highly hydrophobic. The nucleotide sequence of am856_{—}3 indicates that it may contain one or more of the following types of repetitive elements: AT-like, (TTTC)5 short tandem repeat polymorphism UM69.

Clone “am996_{—}12”

A polynucleotide of the present invention has been identified as clone “am996_{—}12”. am996_{—}12 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. am996_{—}12 is a full-length clone, including the entire coding sequence of a secreted protein (also referred to herein as “am996_{—}12 protein”).

The nucleotide sequence of am996_{—}12 as presently determined is reported in SEQ ID NO:23, and includes a poly(A) tail. What applicants presently believe to be the proper reading frame and the predicted amino acid sequence of the am996_{—}12 protein corresponding to the foregoing nucleotide sequence is reported in SEQ ID NO:24. Amino acids 14 to 26 are a predicted leader/signal sequence, with the predicted mature amino acid sequence beginning at amino acid 27. Due to the hydrophobic nature of the predicted leader/signal sequence, it is likely to act as a transmembrane domain should the predicted leader/signal sequence not be separated from the remainder of the am996_{—}12 protein.

The EcoRI/NotI restriction fragment obtainable from the deposit containing clone am996_{—}12 should be approximately 1000 bp.

The nucleotide sequence disclosed herein for am996_{—}12 was searched against the GenBank and GeneSeq nucleotide sequence databases using BLASTN/BLASTX and FASTA search protocols. No hits were found in the database. The TopPredII computer program predicts two potential transmembrane domains within the am996_{—}12 protein sequence, centered around amino acids 18 and 62 of SEQ ID NO:24, respectively.

Clone “cc69_{—}1”

A polynucleotide of the present invention has been identified as clone “cc69_{—}1”. cc69_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cc69_{—}1 is a full-length clone, including the entire coding sequence of a secreted protein (also referred to herein as “cc69_{—}1 protein”).

The nucleotide sequence of cc69_{—}1 as presently determined is reported in SEQ ID NO:25, and includes a poly(A) tail. What applicants presently believe to be the proper reading frame and the predicted amino acid sequence of the cc691 protein corresponding to the foregoing nucleotide sequence is reported in SEQ ID NO:26.

The EcoRI/NotI restriction fragment obtainable from the deposit containing clone cc69_{—}1 should be approximately 550 bp.

The nucleotide sequence disclosed herein for cc69_{—}1 was searched against the GenBank and GeneSeq nucleotide sequence databases using BLASTN/BLASTX and FASTA search protocols. cc69_{—}1 demonstrated at least some similarity with sequences identified as AA280712 (zs98h11.r1 NCI_CGAP_GCB1 Homo sapiens cDNA clone IMAGE:711717 5′), AA421250 (zu27b03.s1 Soares ovary tumor NbHOT Homo sapiens cDNA clone 739181 3′), H28886 (yp03e09.s1 Homo sapiens cDNA clone 186376 3′), and H84171 (yv87c11.r1 Homo sapiens cDNA). Based upon sequence similarity, cc69_{—}1 proteins and each similar protein or peptide may share at least some activity. The TopPredII computer program predicts a potential transmembrane domain within the cc69_{—}1 protein sequence centered around amino acid 15 of SEQ ID NO:26.

Clone “cc162_{—}1”

A polynucleotide of the present invention has been identified as clone “cc162_{—}1”. cc162_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cc162_{—}1 is a full-length clone, including the entire coding sequence of a secreted protein (also referred to herein as “cc162_{—}1 protein”).

The nucleotide sequence of cc162_{—}1 as presently determined is reported in SEQ ID NO:27, and includes a poly(A) tail. What applicants presently believe to be the proper reading frame and the predicted amino acid sequence of the cc162_{—}1 protein corresponding to the foregoing nucleotide sequence is reported in SEQ ID NO:28. Amino acids 2 to 14 are a predicted leader/signal sequence, with the predicted mature amino acid sequence beginning at amino acid 15. Due to the hydrophobic nature of the predicted leader/signal sequence, it is likely to act as a transmembrane domain should the predicted leader/signal sequence not be separated from the remainder of the cc162_{—}1 protein.

The EcoRI/NotI restriction fragment obtainable from the deposit containing clone cc162_{—}1 should be approximately 785 bp.

The nucleotide sequence disclosed herein for cc162_{—}1 was searched against the GenBank and GeneSeq nucleotide sequence databases using BLASTN/BLASTX and FASTA search protocols. cc162_{—}1 demonstrated at least some similarity with sequences identified as AA369067 (EST80419 Placenta II Homo sapiens cDNA 5′ end similar to EST containing Alu repeat), L05367 (Human oligodendrocyte myelin glycoprotein (OMG) exons), and R97898 (yq60b11.r1 Homo sapiens cDNA clone 200157 5′). Based upon sequence similarity, cc162_{—}1 proteins and each similar protein or peptide may share at least some activity. The nucleotide sequence of cc162_{—}1 indicates that it may contain one or more of the following types of repetitive elements: ALU, LI.

Clone “if87_{—}1”

A polynucleotide of the present invention has been identified as clone “if87_{—}1”. if87_{—}1 was isolated from a human adult uterus cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. if87_{—}1 is a full-length clone, including the entire coding sequence of a secreted protein (also referred to herein as “if87_{—}1 protein”).

The nucleotide sequence of if87_{—}1 as presently determined is reported in SEQ ID NO:29, and includes a poly(A) tail. What applicants presently believe to be the proper reading frame and the predicted amino acid sequence of the if87_{—}1 protein corresponding to the foregoing nucleotide sequence is reported in SEQ ID NO:30. Amino acids 8 to 20 are a predicted leader/signal sequence, with the predicted mature amino acid sequence beginning at amino acid 21. Due to the hydrophobic nature of the predicted leader/signal sequence, it is likely to act as a transmembrane domain should the predicted leader/signal sequence not be separated from the remainder of the if87_{—}1 protein.

The EcoRI/NotI restriction fragment obtainable from the deposit containing clone if87_{—}1 should be approximately 900 bp.

The nucleotide sequence disclosed herein for if87_{—}1 was searched against the GenBank and GeneSeq nucleotide sequence databases using BLASTN/BLASTX and FASTA search protocols. if87_{—}1 demonstrated at least some similarity with sequences identified as AA172949 (ms20b07.r1 Stratagene mouse skin (#937313) Mus musculus cDNA clone 607477 5′), AC002310 (Homo sapiens Chromosome 16 BAC clone CIT987-SKA-635H12˜complete genomic sequence, complete sequence), AC003012 (Human PAC clone DJ0169K13, complete sequence), D59442 (Human fetal brain cDNA 3′-end GEN-037G12), R72810 (y109f12.r1 Homo sapiens cDNA clone 157775 5′ similar to contains MSR1 repetitive element), and X74358 (P.camea Pod-EPPT mRNA). The predicted amino acid sequence disclosed herein for if87_{—}1 was searched against the GenPept and GeneSeq amino acid sequence databases using the BLASTX search protocol. The predicted if87_{—}1 protein demonstrated at least some similarity to sequences identified as Z46970 (secreted acid phosphatase 2 (SAP2) [Leishmania mexicana]). Based upon sequence similarity, if87_{—}1 proteins and each similar protein or peptide may share at least some activity. The TopPredII computer program predicts an additional potential transmembrane domain within the if87_{—}1 protein sequence centered around arnino acid 58 of SEQ ID NO:30. The nucleotide sequence of if87_{—}1 indicates that it may contain one or more of the following repetitive elements: ALU, LIMA. if87_{—}1 protein was expressed in a COS cell expression system, and an expressed protein band of approximately 35 kDa was detected in membrane fractions using SDS polyacrylamide gel electrophoresis.

Clone “nn103_{—}4”

A polynucleotide of the present invention has been identified as clone “nn103_{—}4”. nn103_{—}4 was isolated from a human fetal kidney (293 cell line) cDNA library using methods which are selective for cDNAs encoding secreted proteins (see U.S. Pat. No. 5,536,637), orwas identified as encoding a secreted or transmembrane protein on the basis of computer analysis of the amino acid sequence of the encoded protein. nn103_{—}4 is a full-length clone, including the entire coding sequence of a secreted protein (also referred to herein as “nn103_{—}4 protein”).

The nucleotide sequence of nn103_{—}4 as presently determined is reported in SEQ ID NO:31, and includes a poly(A) tail. What applicants presently believe to be the proper reading frame and the predicted amino acid sequence of the nn103_{—}4 protein corresponding to the foregoing nucleotide sequence is reported in SEQ ID NO:32. Amino acids 19 to 31 are a predicted leader/signal sequence, with the predicted mature amino acid sequence beginning at amino acid 32. Due to the hydrophobic nature of the predicted leader/signal sequence, it is likely to act as a transmembrane domain should the predicted leader/signal sequence not be separated from the remainder of the nn103_{—}4 protein.

The EcoRI/NotI restriction fragment obtainable from the deposit containing clone nn103_{—}4 should be approximately 3500 bp. The nucleotide sequence disclosed herein for nn103_{—}4 was searched against the GenBank and GeneSeq nucleotide sequence databases using BLASTN/BLASTX and FASTA search protocols. nn103_{—}4 demonstrated at least some similarity with sequences identified as AA134609 (zn90e04.r1 Stratagene lung carcinoma 937218 Homo sapiens cDNA clone 565470 5′), AA584818 (no09e05.s1 NCI_CGAP_Phel Homo sapiens cDNA clone IMAGE 1100192 simnilar to contains L1.t1 L1 repetitive element), AC002416 (*** SEQUENCING IN PROGRESS *** Human Chromosome X; HTGS phase 1, 3 unordered pieces), AC002456 (Human BAC clone RG013L03 from 7q21, complete sequence), D25252 (Human randomly sequenced mRNA), Q05615 (Insert from pARC 1153), U95743 (Homo sapiens chromosome 16 BAC done CIT987-SK65D3, complete sequence), Z22970 (H.sapiens mRNA for M130 antigen cytoplasmic variant 2), Z71182 (Human DNA sequence from pac 248J6, between markers DXS366 and DXS87 on chromosome X contains STS), Z81310 (Human DNA sequence from cosmid 019A on chromosome 6 Contains HLA DNA gene and STS), Z82253 (Human DNA sequence *** SEQUENCING IN PROGRESS *** from clone U151E3; HTGS phase 1), and Z92547 (Human DNA sequence from PAC 863K). The predicted amino acid sequence disclosed herein for nn103_{—}4 was searched against the GenPept and GeneSeq amino acid sequence databases using the BLASTX search protocol. The predicted nn103_{—}4 protein demonstrated at least some similarity to sequences identified as X52235 (ORFII [Homo sapiens]). Based upon sequence similarity, nn103_{—}4 proteins and each similar protein or peptide may share at least some activity. The TopPredII computer program predicts an additional potential transmembrane domain within the nn103_{—}4 protein sequence centered around amino acid 52 of SEQ ID NO:32. The nucleotide sequence of nn103_{—}4 indicates that it may contain one or more of the following types of repetitive elements: Li, A, MER31.

Clone “np206_{—}8”

A polynucleotide of the present invention has been identified as clone “np206_{—}8”. np206_{—}8 was isolated from a human fetal kidney (293 cell line) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. np206_{—}8 is a full-length clone, including the entire coding sequence of a secreted protein (also referred to herein as “np206_{—}8 protein”). The nucleotide sequence of np206_{—}8 as presently determined is reported in SEQ ID NO:33, and includes a poly(A) tail. What applicants presently believe to be the proper reading frame and the predicted amino acid sequence of the np206_{—}8 protein corresponding to the foregoing nucleotide sequence is reported in SEQ ID NO:34.

The EcoRI/NotI restriction fragment obtainable from the deposit containing clone np206_{—}8 should be approximately 1900 bp.

The nucleotide sequence disclosed herein for np206_{—}8 was searched against the GenBank and GeneSeq nucleotide sequence databases using BLASTN/BLASTX and FASTA search protocols. np206_{—}8 demonstrated at least some similarity with sequences identified as AA126810 (zn87a12.r1 Stratagene lung cDNA), AC000053 (*** SEQUENCING IN PROGRESS *** Human Cosmid Clone 81a12 and 70g8; HTGS phase 2), AC002094 (Genomic sequence from Human 17, complete sequence), AC002431 (Human BAC clone RG180F08 from 7q31, complete sequence), F09069 (H. sapiens partial cDNA sequence; clone c-2we10), G33587 (human STS SHGC-50493), R37071 (yf66a08.s1 Homo sapiens cDNA clone 27020 3′), U91321 (Human chromosome 16p13 BAC clone), Z68746 (Human DNA sequence from cosmid Q27, chromosome region 11p15.5), and Z92846 (Human DNA sequence from cosmid U105G4, between markers DXS366 and DXS87 on chromosome X contains ESTs). Based upon sequence similarity, np206_{—}8 proteins and each similar protein or peptide may share at least some activity. The nucleotide sequence of np206_{—}8 indicates that it may contain one or more of the following types of repetitive elements: Alu/SVA.

Clone “nt746_{—}4”

A polynucleotide of the present invention has been identified as clone “nt746_{—}4”. nt746_{—}4 was isolated from a human adult brain (corpus callosum) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. nt746_{—}4 is a full-length clone, including the entire coding sequence of a secreted protein (also referred to herein as “nt746_{—}4 protein”).

The nucleotide sequence of nt746_{—}4 as presently determined is reported in SEQ ID NO:35, and includes a poly(A) tail. What applicants presently believe to be the proper reading frame and the predicted amino acid sequence of the nt746_{—}4 protein corresponding to the foregoing nucleotide sequence is reported in SEQ ID NO:36.

The EcoRI/NotI restriction fragment obtainable from the deposit containing clone nt746_{—}4 should be approximately 1200 bp.

The nucleotide sequence disclosed herein for nt746_{—}4 was searched against the GenBank and GeneSeq nucleotide sequence databases using BLASTN/BLASTX and FASTA search protocols. nt746_{—}4 demonstrated at least some similarity with sequences identified as AA489740 (aa43c06.r1 Soares NhPu S1 Homo sapiens cDNA clone 823690 5′),J04989 (Bovine alpha 1_{—}3 galactosyltransferase mRNA completed cds),M60263 (Human alpha-1,3-galactosyltransferase (HGT-2) pseudogene), Q74712 (Galactosyl transferase clone), R24770 (yg42c11.r1 Homo sapiens cDNA clone 35316 5′ similar to SP GATR_BOVIN P14769 N-ACETYLLACTOSAMINIDE ALPHA-1,3-GALACTOSYL-TRANSFERASE), and S71333 (alpha 1,3 galactosyltransferase [New Wor1 d monkeys, mermoset lymphoid cell line B95.8, rRNA Partial, 1131 nt]). The predicted amino acid sequence disclosed herein for nt746_{—}4 was searched against the GenPept and GeneSeq amino acid sequence databases using the BLASTX search protocol. The predicted nt746_{—}4 protein demonstrated at least some similarity to sequences identified as M26925 (galactosyltransferase (EC 2.4.1.151) [Mus musculus]), R80016 (Marmoset alpha-1,3-galactosyltransferase), S71333 (alpha 1,3 galactosyltransferase, alpha 1,3GT [New Wor1 d monkeys, mermoset lymphoid cell line B95.8, Peptide, 376 aa] [Platyrrhinil), and W13639 (Murine alpha(1,3)-galactosyltransferase). Based upon sequence similarity, nt746_{—}4 proteins and each similar protein or peptide may share at least some activity. The TopPredII computer program predicts a potential transmembrane domain within the nt746_{—}4 protein sequence centered around amino acid 15 of SEQ ID NO:36. The nucleotide sequence of nt746_{—}4 indicates that it may contain an LTR repetitive element.

nt746_{—}4 protein was expressed in a COS cell expression system, and an expressed protein band of approximately 100 kDa was detected in conditioned medium using SDS polyacrylamide gel electrophoresis.

Clone “pe286_{—}1”

A polynucleotide of the present invention has been identified as clone “pe286_{—}1”. pe286_{—}1 was isolated from a human adult blood (chronic myelogenous leukemia K5) cDNA library using methods which are selective for cDNAs encoding secreted proteins (see U.S. Pat. No.5,536,637), or was identified as encoding a secreted or transmembrane protein on the basis of computer analysis of the amino acid sequence of the encoded protein. pe286_{—}1 is a full-length clone, including the entire coding sequence of a secreted protein (also referred to herein as “pe286_{—}1 protein”).

The nucleotide sequence of pe286_{—}1 as presently determined is reported in SEQ ID NO:37, and includes a poly(A) tail. What applicants presently believe to be the proper reading frame and the predicted amino acid sequence of the pe286_{—}1 protein corresponding to the foregoing nucleotide sequence is reported in SEQ ID NO:38.

The EcoRI/NotI restriction fragment obtainable from the deposit containing clone pe286_{—}1 should be approximately 300 bp.

The nucleotide sequence disclosed herein for pe286_{—}1 was searched against the GenBank and GeneSeq nucleotide sequence databases using BLASTN/BLASTX and FASTA search protocols. pe286_{—}1 demonstrated at least some similarity with sequences identified as AA588854 (no21h03.s1 NCI_CGAP_Pr22 Homo sapiens cDNA clone IMAGE 1101365), L46897 (Homo sapiens (subclone 3_d9 from P1 H13) DNA sequence), and N48057 (yy99d09.s1 Homo sapiens cDNA clone 281681 3′ similar to contains element MER4 repetitive element). Based upon sequence similarity, pe286_{—}1 proteins and each similar protein or peptide may share at least some activity.

Clone “yb7_{—}1”

A polynucleotide of the present invention has been identified as clone “yb7_{—}1”. yb7_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. yb7_{—}1 is a full-length clone, including the entire coding sequence of a secreted protein (also referred to herein as “yb7_{—}1 protein”).

The nucleotide sequence of yb7_{—}1 as presently determined is reported in SEQ ID NO:39, and includes a poly(A) tail. What applicants presently believe to be the proper reading frame and the predicted amino acid sequence of the yb7_{—}1 protein corresponding to the foregoing nucleotide sequence is reported in SEQ ID NO:40.

The EcoRI/NotI restriction fragment obtainable from the deposit containing clone yb7_{—}1 should be approximately 1150 bp.

The nucleotide sequence disclosed herein for yb7_{—}1 was searched against the GenBank and GeneSeq nucleotide sequence databases using BLASTN/BLASTX and FASTA search protocols. yb7_{—}1 demonstrated at least some similarity with sequences identified as N99344 (IMAGE:20090 Homo sapiens cDNA clone 20090). Based upon sequence similarity, yb7_{—}1 proteins and each similar protein or peptide may share at least some activity. The TopPredII computer program predicts a potential transmembrane domain within the yb7_{—}1 protein sequence located around amino acid 52 of SEQ ID NO:40; this domain is also a potential leader/signal sequence with the mature protein beginning at or near amino acid 52 of SEQ ID NO:40.

Clone “am728_{—}60”

A polynucleotide of the present invention has been identified as clone “am728_{—}60”. am728_{—}60 was isolated from a human fetal kidney (293 cell line) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. am728_{—}60 is a full-length clone, including the entire coding sequence of a secreted protein (also referred to herein as “am728_{—}60 protein”).

The nucleotide sequence of am728_{—}60 as presently determined is reported in SEQ ID NO:41. What applicants presently believe to be the proper reading frame and the predicted amino acid sequence of the am728_{—}60 protein corresponding to the foregoing nucleotide sequence is reported in SEQ ID NO:42.

The EcoRI/NotI restriction fragment obtainable from the deposit containing clone am728_{—}60 should be approximately 4333 bp.

The nucleotide sequence disclosed herein for am728_{—}60 was searched against the GenBank and GeneSeq nucleotide sequence databases using BLASTN/BLASTX and FASTA search protocols. am728_{—}60 demonstrated at least some similarity with sequences identified as AA446039 (zw66a08.r1 Soares testis NHT Homo sapiens cDNA clone 781142 5′) and U73682 (Human meningioma-expressed antigen 11 (MEA11) mRNA, partial cds). The predicted amino acid sequence disclosed herein for am728_{—}60 was searched against the GenPept and GeneSeq amino acid sequence databases using the BLASTX search protocol. The predicted am728_{—}60 protein demonstrated at least some similarity to sequences identified as U67884 (melanoma inhibitory activity/condrocyte-derived retinoic acid sensitive protein homolog [Rattus norvegicus]), U73682 (meningioma-expressed antigen 11 [Homo sapiens]), U94780 (MEA6 [Homo sapiens]), and X84707 (melanoma growth regulatory protein [Homo sapiens]). Based upon sequence similarity, am728_{—}60 proteins and each similar protein or peptide may share at least some activity. The TopPredII computer program predicts three potential transmembrane domains within the am728_{—}60 protein sequence, centered around amino adds 300, 370, and 670 of SEQ ID NO:42, respectively.

When expressed in COS cells, am728_{—}60 protein was detected in a membrane fraction from these cells as a band migrating at approximately 200 kD on a denaturing SDS polyacrylamide gel.

Clone “bf377_{—}1”

A polynucleotide of the present invention has been identified as clone 'sf377_{—}1”. bf377_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bf377_{—}1 is a full-length clone, including the entire coding sequence of a secreted protein (also referred to herein as “bf377_{—}1 protein”).

The nucleotide sequence of bf377_{—}1 as presently determined is reported in SEQ ID NO:43, and includes a poly(A) tail. What applicants presently believe to be the proper reading frame and the predicted amino acid sequence of the bf377_{—}1 protein corresponding to the foregoing nucleotide sequence is reported in SEQ ID NO:44. Amnino acids 27 to 39 of SEQ ID NO:44 are a predicted leader/signal sequence, with the predicted mature amino acid sequence beginning at amino acid 40. Due to the hydrophobic nature of the predicted leader/signal sequence, it is likely to act as a transmembrane domain should the predicted leader/signal sequence not be separated from the remainder of the bf377_{—}1 protein.

The EcoRI/NotI restriction fragment obtainable from the deposit containing clone bf377_{—}1 should be approximately 450 bp.

The nucleotide sequence disclosed herein for bf377_{—}1 was searched against the GenBank and GeneSeq nucleotide sequence databases using BLASTN/BLASTX and FASTA search protocols. bf377_{—}1 demonstrated at least some similarity with sequences identified as AA559859 (nl48c05.s1 NCI_CGAP_Pr4 Homo sapiens cDNA clone IMAGE 1043912), AA657838 (nu08b11.s1 NCI_CGAP_Pr2 Homo sapiens cDNA clone IMAGE:1207389 similar to gb:M15990 PROTO-ONCOGENE TYROSINE-PROTEIN KINASE YES (HIJAN)), and R49353 (yg67e07.s1 Homo sapiens cDNA clone 38126 3′ similar to contains MER22 repetitive element). Based upon sequence similarity, bf377_{—}1 proteins and each similar protein or peptide may share at least some activity.

Clone “cw354_{—}1”

A polynucleotide of the present invention has been identified as clone “cw354_{—}1”. cw354_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cw354_{—}1 is a full-length clone, including the entire coding sequence of a secreted protein (also referred to herein as “cw354_{—}1 protein”).

The nucleotide sequence of cw354_{—}1 as presently determined is reported in SEQ ID NO:45, and includes a poly(A) tail. What applicants presently believe to be the proper reading frame and the predicted amino acid sequence of the cw354_protein corresponding to the foregoing nucleotide sequence is reported in SEQ ID NO:46. Amino acids 28 to 40 of SEQ ID NO:46 are a predicted leader/signal sequence, with the predicted mature amino acid sequence beginning at amino acid 41. Due to the hydrophobic nature of the predicted leader/signal sequence, it is likely to act as a transmembrane domain should the predicted leader/signal sequence not be separated from the remainder of the cw354_{—}1 protein.

The EcoRI/NotI restriction fragment obtainable from the deposit containing clone cw354_{—}1 should be approximately 1350 bp.

The nucleotide sequence disclosed herein for cw354_{—}1 was searched against the GenBank and GeneSeq nucleotide sequence databases using BLASTN/BLASTX and FASTA search protocols. cw354_{—}1 demonstrated at least some similarity with sequences identified as D58859 (Human placenta cDNA 5′-end GEN-514B03), H07863 (yl86b05.s1 Homo sapiens cDNA clone 45017 3′), N32178 (yy25b09.s1 Homo sapiens cDNA clone 272249 3′), R81953 (yi98e11.r1 Homo sapiens cDNA clone 147308 5′), and W84437 (zd89d06.s1 Soares fetal heart NbHH19W Homo sapiens cDNA clone 356651 3′). The predicted amino acid sequence disclosed herein for cw354_{—}1 was searched against the GenPept and GeneSeq amino acid sequence databases using the BLASTX search protocol. The predicted cw354_{—}1 protein demonstrated at least some similarity to sequences identified as U39726 (adenosinetriphosphatase [Mycoplasma genitalium]). Based upon sequence similarity, cw354_{—}1 proteins and each similar protein or peptide may share at least some activity.

Clone “nm134_{—}4”

A polynucleotide of the present invention has been identified as clone “nm134_{—}4”. nm134_{—}4 was isolated from a human adult blood (erythroleukemia TF-1) cDNA library using methods which are selective for cDNAs encoding secreted proteins (see U.S. Pat. No.5,536,637), or was identified as encoding a secreted or transmembrane protein on the basis of computer analysis of the amino acid sequence of the encoded protein. nm134_{—}4 is a full-length clone, including the entire coding sequence of a secreted protein (also referred to herein as “nm134_{—}4 protein”).

The nucleotide sequence of nm134_{—}4 as presently determined is reported in SEQ ID NO:47, and includes a poly(A) tail. What applicants presently believe to be the proper reading frame and the predicted amino acid sequence of the nm134_{—}4 protein corresponding to the foregoing nucleotide sequence is reported in SEQ ID NO:48. Amino acids 136 to 148 of SEQ ID NO:48 are a predicted leader/signal sequence, with the predicted mature amino acid sequence beginning at amino acid 149. Due to the hydrophobic nature of the predicted leader/signal sequence, it is likely to act as a transmembrane domain should the predicted leader/signal sequence not be separated from the remainder of the nm134_{—}4 protein. The EcoRI/NotI restriction fragment obtainable from the deposit containing clone nm134_{—}4 should be approximately 1500 bp.

The nucleotide sequence disclosed herein for nm134_{—}4 was searched against the GenBank and GeneSeq nucleotide sequence databases using BLASTN/BLASTX and FASTA search protocols. nm134_{—}4 demonstrated at least some similarity with sequences identified as AA205020 (zq72a12.r1 Stratagene neuroepithelium (#937231) Homo sapiens cDNA clone 647134 5′), AA205286 (zq72a12.s1 Stratagene neuroepithelium (#937231) Homo sapiens cDNA clone 647134 3′), AA261864 (zs18h05.r1 Soares NbHTGBC Homo sapiens cDNA clone 685593 5′), and H63680 (yr55d03.r1 Homo sapiens cDNA clone 209189 5′). Based upon sequence similarity, nm134_{—}4 proteins and each similar protein or peptide may share at least some activity. The TopPredII computer program predicts five potential transmembrane domains within the nm134_{—}4 protein sequence centered around amino acids 108, 132, 170, 195, and 226 of SEQ ID NO:48, respectively.

Clone “yb11_{—}1”

A polynucleotide of the present invention has been identified as clone “yb11_{—}1”. yb11_{—}1 was isolated from a human fetal brain cDNA library and was identified as encoding a secreted or transmembrane protein on the basis of computer analysis of the amino acid sequence of the encoded protein. Yb11_{—}1 is a full-length clone, including the entire coding sequence of a secreted protein (also referred to herein as “yb11_{—}1 protein”). The nucleotide sequence of yb11_{—}1 as presently determined is reported in SEQ ID NO:49, and includes a poly(A) tail. What applicants presently believe to be the proper reading frame and the predicted amino acid sequence of the yb11_{—}1 protein corresponding to the foregoing nucleotide sequence is reported in SEQ ID NO:50. Amino acids 43 to 55 of SEQ ID NO:50 are a predicted leader/signal sequence, with the predicted mature amino acid sequence beginning at amino acid 56. Due to the hydrophobic nature of the predicted leader/signal sequence, it is likely to act as a transmembrane domain should the predicted leader/signal sequence not be separated from the remainder of the yb11_{—}1 protein.

The EcoRI/NotI restriction fragment obtainable from the deposit containing done yb11_{—}1 should be approximately 2800 bp.

The nucleotide sequence disclosed herein for yb11_{—}1 was searched against the GenBank and GeneSeq nucleotide sequence databases using BLASTN/BLASTX and FASTA search protocols. Yb11_{—}1 demonstrated at least some similarity with sequences identified as R55695 (yg88f12.s1 Homo sapiens cDNA clone 40397 3′) and R85100 (yo43b05.s1 Homo sapiens cDNA clone 180657 3′). Based upon sequence similarity, ybll I proteins and each similar protein or peptide may share at least some activity.

Clone “yc2_{—}1”

A polynucleotide of the present invention has been identified as clone “yc2_{—}1”. yc2_{—}1 was isolated from a human fetal kidney (293 cell line) cDNA library and was identified as encoding a secreted or transmembrane protein on the basis of computer analysis of the amino acid sequence of the encoded protein. yc2_{—}1 is a full-length clone, including the entire coding sequence of a secreted protein (also referred to herein as “yc2_{—}1 protein”).

The nucleotide sequence of yc2_{—}1 as presently determined is reported in SEQ ID NO:51, and includes a poly(A) tail. What applicants presently believe to be the proper reading frame and the predicted amino acid sequence of the yc2_{—}1 protein corresponding to the foregoing nucleotide sequence is reported in SEQ ID NO:52. Amino acids 15 to 27 of SEQ ID NO:52 are a predicted leader/signal sequence, with the predicted mature amino acid sequence beginning at amino acid 28. Due to the hydrophobic nature of the predicted leader/signal sequence, it is likely to act as a transmembrane domain should the predicted leader/signal sequence not be separated from the remainder of the yc2_{—}1 protein.

The EcoRI/NotI restriction fragment obtainable from the deposit containing clone yc2_{—}1 should be approximately 2900 bp.

The nucleotide sequence disclosed herein for yc2_{—}1 was searched against the GenBank and GeneSeq nucleotide sequence databases using BLASTN/BLASTX and FASTA search protocols. yc2_{—}1 demonstrated at least some similarity with sequences identified as AA618531 (np38a03.s1 NCI_CGAP_Lul Homo sapiens cDNA clone IMAGE:1118572 similar to contains Alu repetitive element) and AA626937 (af84h07.s1 Soares testis NHT Homo sapiens cDNA clone 1048765 3′). Based upon sequence similarity, yc2_{—}1 proteins and each similar protein or peptide may share at least some activity. The nucleotide sequence of yc2_{—}1 indicates that it may contain one or more Alu repetitive elements.

Clone “ff168_{—}12”

A polynucleotide of the present invention has been identified as clone “ff168_{—}12”. ff168_{—}12 was isolated from a human adult testes (teratocarcinoma NCCIT) cDNA library using methods which are selective for cDNAs encoding secreted proteins (see U.S. Pat. No.5,536,637), or was identified as encoding a secreted or transmembrane protein on the basis of computer analysis of the amino acid sequence of the encoded protein. ff168_{—}12 is a full-length clone, including the entire coding sequence of a secreted protein (also referred to herein as “ff168_{—}12 protein”).

The nucleotide sequence of ff168_{—}12 as presently determined is reported in SEQ ID NO:53, and includes a poly(A) tail. What applicants presently believe to be the proper reading frame and the predicted amino acid sequence of the ff168_{—}12 protein corresponding to the foregoing nucleotide sequence is reported in SEQ ID NO:54.

The EcoRI/NotI restriction fragment obtainable from the deposit containing clone ff168_{—}12 should be approximately 1600 bp.

The nucleotide sequence disclosed herein for ff168_{—}12 was searched against the GenBank and GeneSeq nucleotide sequence databases using BLASTN/BLASTX and FASTA search protocols. ff168_{—}12 demonstrated at least some similarity with sequences identified as AA025945 (ze9le02.r1 Soares fetal heart NbHH19W Homo sapiens cDNA clone 366362 5′), AA156237 (z150c09.s1 Soares pregnant uterus NbHPU Homo sapiens cDNA clone 505360 3′), AA420993 (zu08e09.s1 Soares testis NHT Homo sapiens cDNA clone 731272 3′), N78486 (yz78e03.r1 Homo sapiens cDNA clone 289180 5′), WO1843 (za80a01.r1 Soares fetal lung NbHL19W Homo sapiens cDNA clone 298824 5′), and W95777 (ze07e02.r1 Soares fetal heart NbHH19W Homo sapiens cDNA clone 358298 5′). Based upon sequence similarity, ff168_{—}12 proteins and each similar protein or peptide may share at least some activity.

Clone “ls9_{—}1”

A polynucleotide of the present invention has been identified as clone “ls9_{—}1”. ls9_{—}1 was isolated from a human adult brain (substantia nigra) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ls9_{—}1 is a full-length clone, including the entire coding sequence of a secreted protein (also referred to herein as “ls9_{—}1 protein”).

The nucleotide sequence of ls9_{—}1 as presently determined is reported in SEQ ID NO:55, and includes a poly(A) tail. What applicants presently believe to be the proper reading frame and the predicted amino acid sequence of the ls9_{—}1 protein corresponding to the foregoing nucleotide sequence is reported in SEQ ID NO:56. Amino acids 60 to 72 of SEQ ID NO:56 are a predicted leader/signal sequence, with the predicted mature amino acid sequence beginning at amino acid 73. Due to the hydrophobic nature of the predicted leader/signal sequence, it is likely to act as a transmembrane domain should the predicted leader/signal sequence not be separated from the remainder of the ls9_{—}1 protein.

The EcoRI/Noti restriction fragment obtainable from the deposit containing done ls9_{—}1 should be approximately 2300 bp.

The nucleotide sequence disclosed herein for ls9_{—}1 was searched against the GenBank and GeneSeq nucleotide sequence databases using BLASTN/BLASTX and FASTA search protocols. ls9_{—}1 demonstrated at least some similarity with sequences identified as AA527586 (ng42d05.s1 NCI_CGAP_Co3 Homo sapiens cDNA clone IMAGE:937449), AC000119 (Human BAC clone RG104I04 from 7q21_{—}7q22, complete sequence), T18551 (Human polycystic kidney disease normal PKD1 gene), Y10196 (H.sapiens PEX gene), and Z94721 (Human DNA sequence *** SEQUENCING IN PROGRESS *** from clone 167A14; HTGS phase 1). The predicted amnino acid sequence disclosed herein for ls9_{—}1 was searched against the GenPept and GeneSeq amnino acid sequence databases using the BLASTX search protocol. The predicted ls9_{—}1 protein demonstrated at least some similarity to sequences identified as AB(02375 (KIAA0377 [Homo sapiens]) and R95913 (Neural thread protein). Based upon sequence similarity, ls9_{—}1 proteins and each similar protein or peptide may share at least some activity. The TopPredIl computer program predicts an additional potential transmembrane domain within the ls9_{—}1 protein sequence centered around amino acid 40 of SEQ ID NO:56. The nucleotide sequence of ls9_{—}1 indicates that it may contain an Alu /SVA repetitive element.

Clone “na1010_{—}1”

A polynucleotide of the presentinventionhas been identified as clone “na1010_{—}1”. na1010_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. na1010_{—}1 is a full-length clone, including the entire coding sequence of a secreted protein (also referred to herein as “na1010_{—}1 protein”). The nucleotide sequence of na1010_{—}1 as presently determined is reported in SEQ ID NO:57, and includes a poly(A) tail. What applicants presently believe to be the proper reading frame and the predicted amino acid sequence of the na1010_{—}1 protein corresponding to the foregoing nucleotide sequence is reported in SEQ ID NO:58. Amino acids 24 to 36 of SEQ ID NO:58 are a predicted leader/signal sequence, with the predicted mature amino acid sequence beginning at amino acid 37. Due to the hydrophobic nature of the predicted leader/signal sequence, it is likely to act as a transmembrane domain should the predicted leader/signal sequence not be separated from the remainder of the na1010_{—}1 protein.

The EcoRI/NotI restriction fragment obtainable from the deposit containing clone na1010_{—}1 should be approximately 1050 bp.

The nucleotide sequence disclosed herein for na1010_{—}1 was searched against the GenBank and GeneSeq nucleotide sequence databases using BLASTN/BLASTX and FASTA search protocols. na1010_{—}1 demonstrated at least some similarity with sequences identified as AC002091 (Genomic sequence from Human 17, complete sequence), AC002382 (Human BAC clone RG022J17 from 7q21, complete sequence), and M26434 (Human hypoxanthine phosphoribosyltransferase (HPRT) gene, complete cds). Based upon sequence similarity, na1010_{—}1 proteins and each similar protein or peptide may share at least some activity. The nucleotide sequence of na1010_{—}1 indicates that it may contain one or more of the following repetitive elements: L1/A/MIR/SVA/LTRII, Alu/SVA/A/GAA, or Alu/A/GAAAA.

Clone “nf87_{—}1”

A polynucleotide of the present invention has been identified as clone “nf87_{—}1”. nf87_{—}1 was isolated from a human adult brain (substantia nigra) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. nf87_{—}1 is a full-length clone, including the entire coding sequence of a secreted protein (also referred to herein as “nf87_{—}1 protein”).

The nucleotide sequence of nf87_{—}1 as presently determined is reported in SEQ ID NO:59, and includes a poly(A) tail. What applicants presently believe to be the proper reading frame and the predicted amino acid sequence of the nf87_{—}1 protein corresponding to the foregoing nucleotide sequence is reported in SEQ ID NO:60. Amino acids 53 to 65 of SEQ ID NO:60 are a predicted leader/signal sequence, with the predicted mature amino acid sequence beginning at amino acid 66. Due to the hydrophobic nature of the predicted leader/signal sequence, it is likely to act as a transmembrane domain should the predicted leader/signal sequence not be separated from the remainder of the nf87_{—}1 protein.

The EcoRI/NotI restriction fragment obtainable from the deposit containing clone nf87_{—}1 should be approximately 1200 bp.

The nucleotide sequence disclosed herein for nf87_{—}1 was searched against the GenBank and GeneSeq nucleotide sequence databases using BLASTN/BLASTX and FASTA search protocols. nf87_{—}1 demonstrated at least some similarity with sequences identified as AA358277 (EST67398 Fetal lung III Homo sapiens cDNA 5′ end similar to similar to interferon-alpha-inducible gene p27), W52706 (zc55g02.r1 Soares senescent fibroblasts NbHSF Homo sapiens cDNA clone 326258 5′ similar to SW INI7_HUMAN P40305 INTERFERON-ALPHA INDUCED 11.5 KD PROTEIN), and X67325 (H.sapiens p27 mRNA). The predicted amino acid sequence disclosed herein for nf87_{—}1 was searched against the GenPept and GeneSeq amino acid sequence databases using the BLASTX search protocol. The predicted nf87_{—}1 protein demonstrated at least some similarity to sequences identified as X67325 (p27 gene product [Homo sapiens]). The interferon-alpha-inducible gene is localized on human chromosome 14q32 and expresses the highly hydrophobic p27 gene product in breast carcinoma cells. Based upon sequence similarity, nf87_{—}1 proteins and each similar protein or peptide may share at least some activity. nf87_{—}1 protein was expressed in a COS cell expression system, and an expressed protein band of approximately 16 kDa was detected in membrane fractions using SDS polyacrylamide gel electrophoresis.

Clone “nh796_{—}1”

A polynucleotide of the present invention has been identified as clone “nh796_{—}1”. nh796_{—}1 was isolated from a human adult brain (thalamus) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. nh796_{—}1 is a full-length clone, including the entire coding sequence of a secreted protein (also referred to herein as “nh796_{—}1 protein”).

The nucleotide sequence of nh796_{—}1 as presently determined is reported in SEQ ID NO:61, and includes a poly(A) tail. VVhat applicants presently believe to be the proper reading frame and the predicted amino acid sequence of the nh796_{—}1 protein corresponding to the foregoing nucleotide sequence is reported in SEQ ID NO:62. Amino acids 7 to 19 of SEQ ID NO:62 are a predicted leader/signal sequence, with the predicted mature amino acid sequence beginning at amino acid 20. Due to the hydrophobic nature of the predicted leader/signal sequence, it is likely to act as a transmembrane domain should the predicted leader/signal sequence not be separated from the remainder of the nh796_{—}1 protein.

The EcoRI/NotI restriction fragment obtainable from the deposit containing clone nh796_{—}1 should be approximately 1050 bp.

The nucleotide sequence disclosed herein for nh796_{—}1 was searched against the GenBank and GeneSeq nucleotide sequence databases using BLASTN/BLASTX and FASTA search protocols. nh796_{—}1 demonstrated at least some similarity with sequences identified as AA315985 (EST18772 Lung Homo sapiens cDNA 5′ end), N23239 (yw47b07.s1 Homo sapiens cDNA done 255349 3′), N27741 (yw51c06.s1 Homo sapiens cDNA clone 255754 3′), U69172 (Mus musculus unknown protein mRNA, complete cds), and Z24371 (H. sapiens (D20S195) DNA segment containing (CA) repeat; clone AFM321xc1; single read). The predicted amino acid sequence disclosed herein for nh796_{—}1 was searched against the GenPept and GeneSeq amino acid sequence databases using the BLASTX search protocol. The predicted nh796_{—}1 protein demonstrated at least some similarity to sequences identified as U69172 (unknown [Mus musculus]). The mouse protein of unknown function (U69172) is expressed in late palate development. Based upon sequence similarity, nh796_{—}1 proteins and each similar protein or peptide may share at least some activity.

nh796_{—}1 protein was expressed in a COS cell expression system, and an expressed proteinband of approximately 25 kDa was detected in conditioned media and membrane fractions using SDS polyacrylamide gel electrophoresis.

Clone “nn229_{—}1”

A polynucleotide of the present invention has been identified as clone “nn229_{—}1”. nn229_{—}1 was isolated from a human fetal kidney (293 cell line) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. nn229_{—}1 is a full-length clone, including the entire coding sequence of a secreted protein (also referred to herein as “nn229_{—}1 protein”).

The nucleotide sequence of nn229_{—}1 as presently determined is reported in SEQ ID NO: 63, and includes a poly(A) tail. What applicants presently believe to be the proper reading frame and the predicted amino acid sequence of the nn229_{—}1 protein corresponding to the foregoing nucleotide sequence is reported in SEQ ID NO:64. Amino acids 59 to 71 of SEQ ID NO:64 are a predicted leader/signal sequence, with the predicted mature amino acid sequence beginning at amino acid 72. Due to the hydrophobic nature of the predicted leader/signal sequence, it is likely to act as a transmembrane domain should the predicted leader/signal sequence not be separated from the remainder of the nn229_{—}1 protein.

The EcoRI/NotI restriction fragment obtainable from the deposit containing clone nn229_{—}1 should be approximately 1050 bp.

The nucleotide sequence disclosed herein for nn229_{—}1 was searched against the GenBank and GeneSeq nucleotide sequence databases using BLASTN/BLASTX and FASTA search protocols. nn229_{—}1 demonstrated at least some similarity with sequences identified as H24014 (ym49f02.s1 Homo sapiens cDNA clone 51480 3′), R08508 (ye95h01.r1 Homo sapiens cDNA clone 125521 5′ similar to gb |M87910| HUMALNE34 Human carcinoma cell-derived Alu RNA transcript, (rRNA); gb J02931 TISSUE FACTOR PRECURSOR (HUMAN)), and Z96508 (H.sapiens telomeric DNA sequence, clone 22QTEL030, read 22QTELOO030.seq). Based upon sequence similarity, nn229_{—}1 proteins and each similar protein or peptide may share at least some activity. The TopPredII computer program predicts a potential transmembrane domain within the nn229_{—}1 protein sequence centered around amino acid 20 of SEQ ID NO:64. The nucleotide sequence of nn229_{—}1 indicates that it may contain a MER20 repetitive element.

Clone “np156_{—}1”

A polynucleotide of the present invention has been identified as clone “np156_{—}1”. np156_{—}1 was isolated from a human fetal kidney (293 cell line) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. np156_{—}1 is a full-length clone, including the entire coding sequence of a secreted protein (also referred to herein as “np156_{—}1 protein”). The nucleotide sequence of np156_{—}1 as presently determined is reported in SEQ ID NO:65, and includes a poly(A) tail. What applicants presently believe to be the proper reading frame and the predicted amino acid sequence of the np156_{—}1 protein corresponding to the foregoing nucleotide sequence is reported in SEQ ID NO:66. Amino acids 6 to 18 of SEQ ID NO:66 are a predicted leader/signal sequence, with the predicted mature amino acid sequence beginning at amino acid 19. Due to the hydrophobic nature of the predicted leader/signal sequence, it is likely to act as a transmembrane domain should the predicted leader/signal sequence not be separated from the remainder of the np156_{—}1 protein.

The EcoRI/NotI restriction fragment obtainable from the deposit containing clone np156_{—}1 should be approximately 1200 bp.

The nucleotide sequence disclosed herein for np156_{—}1 was searched against the GenBank and GeneSeq nucleotide sequence databases using BLASTN/BLASTX and FASTA search protocols. np156_{—}1 demonstrated at least some similarity with sequences identified as AA298580 (EST114211 HSC172 cells I Homo sapiens cDNA 5′ end), AA447514 (zw81a05.s1 Soares testis NHT Homo sapiens cDNA clone 782576 3′), AC002309 (*e* SEQUENCING IN PROGRESS *** Human Chromosome 22qll Cosmid Clone 63e9; HTGS phase 1, 3 unordered pieces), AF007269 (Arabidopsis thaliana BAC IG002N01), and N53641 (yz04g03.r1 Homo sapiens cDNA clone 282100 5′). Based upon sequence similarity, np156_{—}1 proteins and each similar protein or peptide may share at least some activity.

Clone “bg570_{—}1”

A polynucleotide of the present invention has been identified as clone “bg570_{—}1”. bg570_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bg570_{—}1 is a full-length clone, including the entire coding sequence of a secreted protein (also referred to herein as “bg570_{—}1 protein”).

The nucleotide sequence of bg570_{—}1 as presently determined is reported in SEQ ID NO:67, and includes a poly(A) tail. What applicants presently believe to be the proper reading frame and the predicted amino acid sequence of the bg570_{—}1 protein corresponding to the foregoing nucleotide sequence is reported in SEQ ID NO:68. Amino acids 33 to 45 of SEQ ID NO:68 are a predicted leader/signal sequence, with the predicted mature amino acid sequence beginning at amino acid 46. Due to the hydrophobic nature of the predicted leader/signal sequence, it is likely to act as a transmembrane domain should the predicted leader/signal sequence not be separated from the remainder of the bg570_{—}1 protein.

The EcoRI/NotI restriction fragment obtainable from the deposit containing clone bg570_{—}1 should be approximately 900 bp.

The nucleotide sequence disclosed herein for bg570_{—}1 was searched against the GenBank and GeneSeq nucleotide sequence databases using BLASTN/BLASTX and FASTA search protocols. bg570_{—}1 demonstrated at least some similarity with sequences identified as T03370 (IB1429 Infant brain, Bento Soares Homo sapiens cDNA clone IB1429 3′ end). Based upon sequence similarity, bg570_{—}1 proteins and each similar protein or peptide may share at least some activity.

Clone “bi120_{—}2”

A polynucleotide of the present invention has been identified as clone “bi120_{—}2”. bi120_{—}2 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bi120_{—}2 is a full-length clone, including the entire coding sequence of a secreted protein (also referred to herein as “bi120_{—}2 protein”).

The nucleotide sequence of bi120_{—}2 as presently determined is reported in SEQ ID NO:69, and includes a poly(A) tail. What applicants presently believe to be the proper reading frame and the predicted amino acid sequence of the bi120_{—}2 protein corresponding to the foregoing nucleotide sequence is reported in SEQ ID NO:70. Amino acids 39 to 51 of SEQ ID NO:70 are a predicted leader/signal sequence, with the predicted mature amino acid sequence beginning at amino acid 52. Due to the hydrophobic nature of the predicted leader/signal sequence, it is likely to act as a transmembrane domain should the predicted leader/signal sequence not be separated from the remainder of the bi120_{—}2 protein.

The EcoRI/NotI restriction fragment obtainable from the deposit containing clone bi120_{—}2 should be approximately 1800 bp. The nucleotide sequence disclosed herein for bi120_{—}2 was searched against the GenBank and GeneSeq nucleotide sequence databases using BLASTN/BLASTX and FASTA search protocols. bi120_{—}2 demonstrated at least some similarity with sequences identified as AA232119 (zr24a12.r1 Stratagene NT2 neuronal precursor 937230 Homo sapiens cDNA clone 664318 5′ similar to WP:C11H1.2 CE05261), D20759 (Human HL60 3′ directed MboI cDNA, HUMGS01738, clone mp1051), N28753 (yx67h11.r1 Homo sapiens cDNA clone), N28806 (yx70g12.r1 Homo sapiens cDNA clone 267142 5′), N35232 (yy21d02.s1 Homo sapiens cDNA clone 271875 3′), W73805 (zd50g02.r1 Soares fetal heart NbHH19W Homo sapiens cDNA clone 344114 5′), Z61133 (H.sapiens CpG island DNA genomic Msel fragment, clone 45gl, forward read cpg45gl.ftla), and Z70205 (Caenorhabditis elegans cosmid C11H1, complete sequence). bi120_{—}2 also demonstrated at least some similarity with CpG island DNA. The predicted amino acid sequence disclosed herein for bi120_{—}2 was searched against the GenPept and GeneSeq amino acid sequence databases using the BLASTX search protocol. The predicted bi120_{—}2 protein demonstrated at least some similarity to sequences identified as Z70205 (C11H1.2 [Caenorhabditis elegans]). Based upon sequence similarity, bi120_{—}2 proteins and each similar protein or peptide may share at least some activity. The TopPredII computer program predicts five additional potential transmembrane domains within the bi120_{—}2 protein sequence, centered around amino acids 20,80,110,150, and 290 of SEQ ID NO:70, respectively. There may be a frameshift in the full-clone sequence (somewhere within base pairs 99-1010 of SEQ ID NO:69). This frameshift from reading frame 3 to reading frame 1 would extend the open reading frame from 309 amino acids to at least 460 amino acids and add three more potential transmembrane domains to the protein. There also appears to be another frameshift occuring around base pair 1450 of SEQ ID NO:69 which shifts the open reading frame back into frame 3, adding approximately 20 more codons to the open reading frame sequence.

Clone “bn594_{—}1”

A polynucleotide of the present invention has been identified as clone “bn594_{—}1”. bn594_{—}1 was isolated from a human adult placenta cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bn594_{—}1 is a full-length clone, including the entire coding sequence of a secreted protein (also referred to herein as “bn594 1 protein”).

The nucleotide sequence of bn594_{—}1 as presently determined is reported in SEQ ID NO:71, and includes a poly(A) tail. What applicants presently believe to be the proper reading frame and the predicted amino acid sequence of the bn594_{—}1 protein corresponding to the foregoing nucleotide sequence is reported in SEQ ID NO:72.

The EcoRI/NotI restriction fragment obtainable from the deposit containing clone bn594_{—}1 should be approximately 1400 bp.

The nucleotide sequence disclosed herein for bn594_{—}1 was searched against the GenBank and GeneSeq nucleotide sequence databases using BLASTN/BLASTX and FASTA search protocols. bn594_{—}1 demonstrated at least some similarity with sequences identified as J03071 (Human growth hormone (GH-1 and GH-2) and chorionic somatomammotropin (CS-1, CS2 and CS-5) genes, complete cds). Based upon sequence similarity, bn594_{—}1 proteins and each similar protein or peptide may share at least some activity. The TopPredil computer program predicts a potential transmembrane domain within the bn594_{—}1 protein sequence centered around amino acid 52 of SEQ ID NO:72; this region is also a potential signal sequence, with the mature protein starting at amino acid 53 of SEQ ID NO:72. The nucleotide sequence of bn594_{—}1 indicates that it may contain one or more of the following types of repetitive elements: ALU, GAAA.

Clone “en554_{—}1”

A polynucleotide of the present invention has been identified as clone “en554_{—}1”. en554_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. en554_{—}1 is a full-length clone, including the entire coding sequence of a secreted protein (also referred to herein as en554_{—}1 protein”). The nucleotide sequence of en554_{—}1 as presently determined is reported in SEQ ID NO:73, and includes a poly(A) tail. What applicants presently believe to be the proper reading frame and the predicted amino acid sequence of the en554 1 protein corresponding to the foregoing nucleotide sequence is reported in SEQ ID NO:74. Amino acids 15 to 27 of SEQ ID NO:74 are a predicted leader/signal sequence, with the predicted mature amino acid sequence beginning at amino acid 28. Due to the hydrophobic nature of the predicted leader/signal sequence, it is likely to act as a transmembrane domain should the predicted leader/signal sequence not be separated from the remainder of the en554_{—}1 protein.

The EcoRI/NotI restriction fragment obtainable from the deposit containing clone en554_{—}1 should be approximately 1800 bp.

The nucleotide sequence disclosed herein for en554_{—}1 was searched against the GenBank and GeneSeq nucleotide sequence databases using BLASTN/BLASTX and FASTA search protocols. en554_{—}1 demonstrated at least some similarity with sequences identified as AA625842 (zv87d08.s1 Soares N MPu S1 Homo sapiens cDNA clone 766767 3′) and R54550 (yg75h06.r1 Homo sapiens cDNA clone 39297 5′). Based upon sequence similarity, en554_{—}1 proteins and each similar protein or peptide may share at least some activity. The nucleotide sequence of en554_{—}1 indicates that it may contain repetitive elements in the region between base pairs 849 and 1023 of SEQ ID NO:73.

Clone “na474_{—}10”

A polynucleotide of the presentinvention hasbeen identified as clone “na474_{—}10”. na474_{—}10 was isolated from a human adult brain (corpus callosum) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. na474_{—}10 is a full-length clone, including the entire coding sequence of a secreted protein (also referred to herein as “na474_{—}10 protein”).

The nucleotide sequence of na474_{—}10 as presently determined is reported in SEQ ID NO:75, and includes a poly(A) tail. What applicants presently believe to be the proper reading frame and the predicted amino acid sequence of the na474_{—}10 protein corresponding to the foregoing nucleotide sequence is reported in SEQ ID NO:76. Amino acids 69 to 81 of SEQ ID NO:76 are a predicted leader/signal sequence, with the predicted mature amino acid sequence beginning at amino acid 82. Due to the hydrophobic nature of the predicted leader/signal sequence, it is likely to act as a transmembrane domain should the predicted leader/signal sequence not be separated from the remainder of the na474_{—}10 protein.

The EcoRI/NotI restriction fragment obtainable from the deposit containing clone na474_{—}10 should be approximately 1500 bp. The nucleotide sequence disclosed herein for na474_{—}10 was searched against the GenBank and GeneSeq nucleotide sequence databases using BLASTN/BLASTX and FASTA search protocols. na474_{—}10 demonstrated at least some similarity with sequences identified as AA262604 (zs23f01.s1 NCI_CGAP_GCB1 Homo sapiens cDNA clone IMAGE:686041 3′ similar to contains Alu repetitive element), AA450131 (zx42a02.r1 Soares total fetus Nb2HF8 9w Homo sapiens cDNA clone 789098 5′), U72661 (Human ninjurinl mRNA, complete cds), and W38567 (zb20h04.r1 Soares fetal lung NbHL19W Homo sapiens cDNA clone 302647 5′). The predicted amino acid sequence disclosed herein for na474_{—}10 was searched against the GenPept and GeneSeq amino acid sequence databases using the BLASTX search protocol. The predicted na474_{—}10 protein demonstrated at least some similarity to sequences identified as U72661 (ninjurinl [Homo sapiens]). Based upon sequence similarity, na474_{—}10 proteins and each similar protein or peptide may share at least some activity. Ninjurin is a cell-surface protein and adhesion molecule which is induced by nerve injury and promotes axonal growth.

Ninjurin is capable of mediating homophilic adhesion and can promote neurite extension of dorsal root ganglion neurons in vitro. It is thought to play a role in nerve regeneration and in the formation and function of other tissues (Araki et al., 1996, Neuron 17(2):353_{—}361, incorporated herein by reference). na474_{—}10 and ninjurin appear to define a novel family of adhesion molecules. na474_{—}10 protein was expressed in a COS cell expression system, and an expressed protein band of approximately 15 kDa was detected in membrane fractions using SDS polyacrylamide gel electrophoresis.

Clone “nn16_{—}10”

A polynucleotide of the present invention has been identified as clone “nn16_{—}10”. nn16_{—}10 was isolated from a human fetal kidney (293 cell line) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. nn16_{—}10 is a full-length clone, including the entire coding sequence of a secreted protein (also referred to herein as “nn16_{—}10 protein”).

The nucleotide sequence of nn16_{—}10 as presently determined is reported in SEQ ID NO:77, and includes a poly(A) tail. What applicants presently believe to be the proper reading frame and the predicted amino acid sequence of the nn16_{—}10 protein corresponding to the foregoing nucleotide sequence is reported in SEQ ID NO:78. Amino acids 14 to 26 of SEQ ID NO:78 are a predicted leader/signal sequence, with the predicted mature amino acid sequence beginning at amino acid 27. Due to the hydrophobic nature of the predicted leader/signal sequence, it is likely to act as a transmembrane domain should the predicted leader/signal sequence not be separated from the remainder of the nn16_{—}10 protein.

The EcoRI/NotI restriction fragment obtainable from the deposit containing clone nn16_{—}10 should be approximately 1600 bp.

The nucleotide sequence disclosed herein for nn6_{—}10 was searched against the GenBank and GeneSeq nucleotide sequence databases using BLASTN/BLASTX and FASTA search protocols. nn16_{—}10 demonstrated at least some simnilarity with sequences identified as R46973 (Y224 Rattus norvegicus cDNA clone Y224 5′ end), U43404 (Sus scrofa ameloblastin mRNA, complete cds), W13000 (mb21d12.r1 Soares mouse p3NMF19.5 Mus musculus cDNA clone 330071 5′), and W36463 (mb71c12.r1 Soares mouse p3NMF19.5 Mus musculus cDNA clone 334870 5′). The predicted amino acid sequence disclosed herein for nn16_{—}10 was searched against the GenPept and GeneSeq amino acid sequence databases using the BLASTX search protocol. The predicted nn16_{—}10 protein demonstrated at least some similarity to sequences identified as U43404 (ameloblastin [Sus scrofa]), and to the amelobalstin proteins of rat (and other species).

Ameloblastin is a unique ameloblast-specific gene product that may be important in enamel matrix formation and mineralization (Krebsbach et al., 1996, J. Biol. Chem. 271: 4431, incorporated herein by reference). Rat ameloblastin is 442 amino acids and is a tooth-specific enamel matrix protein. Immunohistochemical data show staining of golgi and of secretory granules of the secretory ameloblast, in addition to the entire thickness of the enamel matrix. The rat ameloblastin protein is synthesized as a 55 kDa core protein which undergoes extensive post-translational modifications with O-linked oligo-saccharides to become the 65 kDa secretory form (Uchida et al., 1997, J. Histochem. Cytochem. 45(10):1329-1340, incorporated herein by reference). Based upon sequence similarity, nn16_{—}10 proteins and each similar protein or peptide may share at least some activity.

nn16_{—}10 protein was expressed in a COS cell expression system, and an expressed protein band of approximately 84 kDa was detected in conditioned medium and membrane fractions using SDS polyacrylamide gel electrophoresis.

Clone “np189_{—}9”

A polynucleotide of the present invention has been identified as clone “np189_{—}9”. np189_{—}9 was isolated from a human fetal kidney (293 cell line) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. np189_{—}9 is a full-length clone, including the entire coding sequence of a secreted protein (also referred to herein as “np189_{—}9 protein”). The nucleotide sequence of npl89_{—}9 as presently determined is reported in SEQ ID NO:79, and includes a poly(A) tail. What applicants presently believe to be the proper reading frame and the predicted amino acid sequence of the npl89_{—}9 protein corresponding to the foregoing nucleotide sequence is reported in SEQ ID NO:80. Amino acids 41 to 53 of SEQ ID NO:80 are a predicted leader/signal sequence, with the predicted mature amino acid sequence beginning at amino acid 54. Due to the hydrophobic nature of the predicted leader/signal sequence, it is likely to act as a transmembrane domain should the predicted leader/signal sequence not be separated from the remainder of the npl89 9 protein.

The EcoRI/NotI restriction fragment obtainable from the deposit containing clone np189_{—}9 should be approximately 2100 bp.

The nucleotide sequence disclosed herein for np89_{—}9 was searched against the GenBank and GeneSeq nucleotide sequence databases using BLASTN/BLASTX and FASTA search protocols. np89_{—}9 demonstrated at least some similarity with sequences identified as AA035196 (zk27f12.s1 Soares pregnant uterus NbHPU Homo sapiens cDNA clone 471791 3′), AA336568 (EST41447 Endometrial tumor Homo sapiens cDNA 5′ end), AA420972 (zt86a11.s1 Soares testis NHT Homo sapiens cDNA clone 729212 3′), and H38460 (yp69h08.s1 Homo sapiens cDNA clone 192735 3′). Based upon sequence similarity, np189_{—}9 proteins and each similar protein or peptide may share at least some activity. The TopPredII computer program predicts an additional potential transmembrane domain within the np189_{—}9 protein sequence centered around amino acid 38 of SEQ ID NO:80.

Clone “nv226_{—}1”

A polynucleotide of the present invention has been identified as clone “ny226_{—}1”. ny226_{—}1 was isolated from a human adult brain (substantia nigra) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ny226_{—}1 is a full-length clone, including the entire coding sequence of a secreted protein (also referred to herein as “ny226_{—}1 protein”).

The nucleotide sequence of ny226_{—}1 as presently determined is reported in SEQ ID NO:81, and includes a poly(A) tail. What applicants presently believe to be the proper reading frame and the predicted amino acid sequence of the ny226_{—}1 protein corresponding to the foregoing nucleotide sequence is reported in SEQ ID NO:82.

The EcoRI/NotI restriction fragment obtainable from the deposit containing clone ny226_{—}1 should be approximately 3175 bp.

The nucleotide sequence disclosed herein for ny226_{—}1 was searched against the GenBank and GeneSeq nucleotide sequence databases using BLASTN/BLASTX and FASTA search protocols. ny226_{—}1 demonstrated at least some similarity with sequences identified as AC002463 (Human BAC clone RG302F04 from 7q31, complete sequence), R07637 (ye98e03.s1 Homo sapiens cDNA clone 125788 3′), and Z78730 (H.sapiens flow-sorted chromosome 6 HindIII fragment, SC6pA15C3). Based upon sequence similarity, ny226_{—}1 proteins and each similar protein or peptide may share at least some activity. The TopPredII computer program predicts a potential transmembrane domain within the ny226_{—}1 protein sequence centered around amino acid 22 of SEQ ID NO:82; this region is also a putative signal sequence, with the mature protein starting at amino acid 23 of SEQ ID NO:82. The nucleotide sequence of ny226_{—}1 indicates that it may contain one or more repetitive elements, including ALU repetitive elements.

Clone “pe159_{—}1”

A polynucleotide of the present invention has been identified as clone “pe159_{—}1”. pe159_{—}1 was isolated from a human adult blood (chronic myelogenous leukemia K5) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. pe159_{—}1 is a full-length clone, including the entire coding sequence of a secreted protein (also referred to herein as “pe159_{—}1 protein”).

The nucleotide sequence of pe159_{—}1 as presently determined is reported in SEQ ID NO:83, and includes a poly(A) tail. What applicants presently believe to be the proper reading frame and the predicted amino acid sequence of the pe159_{—}1 protein corresponding to the foregoing nucleotide sequence is reported in SEQ ID NO:84.

The EcoRI/NotI restriction fragment obtainable from the deposit containing clone pe159_{—}1 should be approximately 1000 bp.

The nucleotide sequence disclosed herein for pe159_{—}1 was searched against the GenBank and GeneSeq nucleotide sequence databases using BLASTN/BLASTX and FASTA search protocols. pe159_{—}1 demonstrated at least some similarity with sequences identified as AA372974 (EST84925 Colon adenocarcinoma IV Homo sapiens cDNA 5′ end), AC002377 (Human PAC clone DJ222H05), AC002519 (*** SEQUENCING IN PROGRESS *** Human chromosome 16p11.2 BAC clone CIT987SK-A-355G7; HTGS phase 2, 1 ordered pieces), H45355 (yn99b01.r1 Homo sapiens cDNA clone 176521 5′), W39648 (zc19c09.r1 Soares parathyroid tumor NbHPA Homo sapiens cDNA clone 322768 5′), and Z84816 (Human DNA sequence from PAC 2A2 on chromosome X contains ESTs). The predicted amino acid sequence disclosed herein for pe159_{—}1 was searched against the GenPept and GeneSeq amino acid sequence databases using the BLASTX search protocol. The predicted pe159_{—}1 protein demonstrated at least some similarity to sequences identified as M84237 (integrin beta-i subunit [Homo sapiens]) and R96800 (Human histiocyte-secreted factor HSF). Based upon sequence similarity, pel59_{—}1 proteins and each similar protein or peptide may share at least some activity. The nucleotide sequence of pel59_{—}1 indicates that it may contain one or more of the following types of repetitive elements: Alu, SVA, MER3.

Clone “pj314₈”

A polynucleotide of the present invention has been identified as clone “pj314_{—}8”. pj314_{—}8 was isolated from a human fetal carcinoma (cell type NTD2 treated with retinoic acid for 23 days) cDNA library using methods which are selective for cDNAs encoding secreted proteins (see U.S. Pat. No.5,536,637), or was identified as encoding a secreted or transmembrane protein on the basis of computer analysis of the amino acid sequence of the encoded protein. pj314_{—}8 is a full-length clone, including the entire coding sequence of a secreted protein (also referred to herein as “pj314_{—}8 protein”).

The nucleotide sequence of pj314_{—}8 as presently determined is reported in SEQ ID NO:85, and includes a poly(A) tail. What applicants presently believe to be the proper reading frame and the predicted amino acid sequence of the pj314_{—}8 protein corresponding to the foregoing nucleotide sequence is reported in SEQ ID NO:86. Amino acids 23 to 35 of SEQ ID NO:86 are a predicted leader/signal sequence, with the predicted mature amino acid sequence beginning at amino acid 36. Due to the hydrophobic nature of the predicted leader/signal sequence, it is likely to act as a transmembrane domain should the predicted leader/signal sequence not be separated from the remainder of the pj314_{—}8 protein.

The EcoRI/NotI restriction fragment obtainable from the deposit containing clone pj314_{—}8 should be approximately 1200 bp.

The nucleotide sequence disclosed herein for pj314_{—}8 was searched against the GenBank and GeneSeq nucleotide sequence databases using BLASTN/BLASTX and FASTA search protocols. pj314_{—}8 demonstrated at least some simnilarity with sequences identified as H98510 (yv90g02.r1 Homo sapiens cDNA clone), U03019 (Human melanoma growth stimulatory activity beta (MGSA beta) gene, partial cds), U25660 (Dictyostelium discoideum actin gene, partial cds), W67504 (zd40f09.s1 Soares fetal heart NbHH19W Homo sapiens cDNA clone 343145 3′), Z99358 (Homo sapiens mRNA; expressed sequence tag; clone DKFZphamy1_{—a}3, 5′ read), and Z99359 (Homo sapiens mRNA; expressed sequence tag; clone DKFZphamy1_a3, 3′ read). The predicted amino acid sequence disclosed herein for pj314_{—}8 was searched against the GenPept and GeneSeq amino acid sequence databases using the BLASTX search protocol. The predicted pj314_{—}8 protein demonstrated at least some similarity to sequences identified as U16359 (nitric oxide synthase [Rattus norvegicus]). Based upon sequence similarity, pj314_{—}8 proteins and each similar protein or peptide may share at least some activity. The nucleotide sequence of pj314_{—}8 indicates that it may contain one or more of the following types of repetitive elements: AC repeats, PAB repeats, CA repeats.

Clone “bp870_{—}1”

A polynucleotide of the present invention has been identified as clone “bp870_{—}1”. bp870_{—}1 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bp870_{—}1 is a full-length clone, including the entire coding sequence of a secreted protein (also referred to herein as “bp870_{—}1 protein”).

The nucleotide sequence of bp870_{—}1 as presently determined is reported in SEQ ID NO:87, and includes a poly(A) tail. What applicants presently believe to be the proper reading frame and the predicted amino acid sequence of the bp870_{—}1 protein corresponding to the foregoing nucleotide sequence is reported in SEQ ID NO:88. Amino acids 9 to 21 of SEQ ID NO:88 are a predicted leader/signal sequence, with the predicted mature amino acid sequence beginning at amino acid 22. Due to the hydrophobic nature of the predicted leader/signal sequence, it is likely to act as a transmembrane domain should the predicted leader/signal sequence not be separated from the remainder of the bp870_{—}1 protein.

The EcoRI/NotI restriction fragment obtainable from the deposit containing clone bp870_{—}1 should be approximately 1000 bp.

The nucleotide sequence disclosed herein for bp870_{—}1 was searched against the GenBank and GeneSeq nucleotide sequence databases using BLASTN/BLASTX and FASTA search protocols. bp870_{—}1 demonstrated at least some similarity with sequences identified as AA229935 (nc51g10.r1 NCI_CGAP_Pr3 Homo sapiens cDNA clone IMAGE:1011714 similar to contains Alu repetitive element;contains element MER4 repetitive element), Hi2643 (yj13a04.r1 Homo sapiens cDNA clone 1485905′), and H12594 (yj13a04.s1 Homo sapiens cDNA clone 148590 3′ similar to contains Alu repetitive element). Based upon sequence similarity, bp870_{—}1 proteins and each similar protein or peptide may share at least some activity. The nucleotide sequence of bp870_{—}1 indicates that it may contain a simple repeat region and at least one copy of an Alu repetitive element. bp870_{—}1 protein was expressed in a COS cell expression system, and an expressed protein band of approximately 23 kDa was detected in conditioned medium and membrane fractions using SDS polyacrylamide gel electrophoresis.

Clone “bx141_{—}2”

A polynucleotide of the present invention has been identified as clone “bx141_{—}2”. bx141_{—}2 was isolated from a human adult ovary (PA-1 teratocarcinoma, pool of retinoic-acid-treated, activin-treated, and untreated tissue) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bx141_{—}2 is a full-length clone, including the entire coding sequence of a secreted protein (also referred to herein as “bx141_{—}2 protein”).

The nucleotide sequence of bx141_{—}2 as presently determined is reported in SEQ ID NO:89, and includes a poly(A) tail. What applicants presently believe to be the proper reading frame and the predicted amino acid sequence of the bxi41_{—}2 protein corresponding to the foregoing nucleotide sequence is reported in SEQ ID NO:90. Amino acids 30 to 42 of SEQ ID NO:90 are a predicted leader/signal sequence, with the predicted mature amino acid sequence beginning at amino acid 43. Due to the hydrophobic nature of the predicted leader/signal sequence, it is likely to act as a transmembrane domain should the predicted leader/signal sequence not be separated from the remainder of the bx1412 protein.

The EcoRI/NotI restriction fragment obtainable from the deposit containing clone bx141_{—}2 should be approximately 1800 bp. The nucleotide sequence disclosed herein for bx141_{—}2 was searched against the GenBank and GeneSeq nucleotide sequence databases using BLASTN/BLASTX and FASTA search protocols. bx141_{—}2 demonstrated at least some similarity with sequences identified as AA173353 (zp32b01.r1 Stratagene neuroepithelium (#937231) Homo sapiens cDNA clone 611113 5′ similar to SW:A15_{—HUMAN} P41732 CELL SURFACE GLYCOPROTEIN A15), AA375927 (EST88303 HSC172 cells IIHomo sapiens cDNA 5′ end similar to similar to cell surface glycoprotein), D10653 (Human mRNA for cell surface glycoprotein, complete cds), H64050 (yr58c07.r1 Homo sapiens cDNA clone 209484 5′ similar to SP:S39262 S39262 PLATELET CELL SURFACE GLYCOPROTEIN), and R41866 (yg12f04.s1 Homo sapiens cDNA clone 31854 3′). The predicted amino acid sequence disclosed herein for bx141_{—}2 was searched against the GenPept and GeneSeq amino acid sequence databases using the BLASTX search protocol. The predicted bx141_{—}2 protein demonstrated at least some similarity to sequences identified as D10653 (HUMA15_{—}1 cell surface glycoprotein [Homo sapiens]) and D29808 (HUMTALLA1_{—}1 TALLA_{—}1 [Homo sapiens]). The human cell surface glycoprotein (“D10653 protein”) is a protein of 244 amino acids which contains four potential transmembrane domains and four possible N-linked glycosylation sites. A computer-aided comparison showed a marked similarity between D10653 protein and several other membrane proteins: CD9, CD37, CD53, TAPA-1, Sm23, CO-029, and ME491/CD63; also, D10653 protein is similar to the ME491/CD63 protein superfamily. bx141_{—}2 protein also shows some similarity to the human and mouse ME491 and CD63 proteins. Based upon sequence similarity, bx141_{—}2 proteins and each similar protein or peptide may share at least some activity. The TopPredII computer program predicts four potential transmembrane domains within the bx141_{—}2 protein sequence centered around amino acids 31, 70, 104, and 222 of SEQ ID NO:90, respectively. bx141_{—}2 protein was expressed in a COS cell expression system, and an expressed protein band of approximately 24 kDa was detected in membrane fractions using SDS polyacrylamide gel electrophoresis.

Clone “cw272_{—}7”

A polynucleotide of the present invention has been identified as clone “cw272_{—}7”. cw272_{—}7 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cw272_{—}7 is a full-length clone, including the entire coding sequence of a secreted protein (also referred to herein as “cw272 7 protein”).

The nucleotide sequence of cw272_{—}7 as presently determined is reported in SEQ ID NO:91, and includes a poly(A) tail. What applicants presently believe to be the proper reading frame and the predicted amino acid sequence of the cw272_{—}7 protein corresponding to the foregoing nucleotide sequence is reported in SEQ ID NO:92. Amino acids 48 to 60 of SEQ ID NO:92 are a predicted leader/signal sequence, with the predicted mature amino acid sequence beginning at amino acid 61. Due to the hydrophobic nature of the predicted leader/signal sequence, it is likely to act as a transmembrane domain should the predicted leader/signal sequence not be separated from the remainder of the cw272 7 protein.

The EcoRI/NotI restriction fragment obtainable from the deposit containing clone cw272_{—}7 should be approximately 2300 bp.

The nucleotide sequence disclosed herein for cw272_{—}7 was searched against the GenBank and GeneSeq nucleotide sequence databases using BLASTN/BLASTX and FASTA search protocols. While no clear hits were found in these databases, cw272_{—}7 protein does show some similarity to bone morphogenetic proteins and procollagens.

Clone “nh328_{—}5”

A polynucleotide of the present invention has been identified as clone “nh328_{—}5”. nh328_{—}5 was isolated from a human adult brain (thalamus) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. nh328_{—}5 is a full-length clone, including the entire coding sequence of a secreted protein (also referred to herein as “nh328_{—}5 protein”).

The nucleotide sequence of nh328_{—}5 as presently deternined is reported in SEQ ID NO:93, and includes a poly(A) tail. What applicants presently believe to be the proper reading frame and the predicted amino acid sequence of the nh328_{—}5 protein corresponding to the foregoing nucleotide sequence is reported in SEQ ID NO:94. Amino acids 60 to 72 of SEQ ID NO:94 are a predicted leader/signal sequence, with the predicted mature amino acid sequence beginning at amino acid 73. Due to the hydrophobic nature of the predicted leader/signal sequence, it is likely to act as a transmembrane domain should the predicted leader/signal sequence not be separated from the remainder of the nh328 5 protein.

The EcoRI/NotI restriction fragment obtainable from the deposit containing clone nh328_{—}5 should be approximately 2200 bp.

The nucleotide sequence disclosed herein for nh328_{—}5 was searched against the GenBank and GeneSeq nucleotide sequence databases using BLASTN/BLASTX and FASTA search protocols. nh328_{—}5 demonstrated at least some similarity with sequences identified as AA426157 (zv83aO9.r1 Soares total fetus Nb2HF8 9w Homo sapiens cDNA clone 760216 5′), D17160 (Human HepG2 3′ region MboI cDNA, clone hmd2d01m3), D56329 (Human fetal brain cDNA 5′-end GEN-424F08), N62903 (yy67e09.s1 Homo sapiens cDNA clone 278632 3′), R88485 (ym94e01.r1 Homo sapiens cDNA clone 166584 5′), and T26592 (AB329E6R Homo sapiens cDNA clone LLAB329E6 5′). Based upon sequence similarity, nh328 5 proteins and each similar protein or peptide may share at least some activity. The nucleotide sequence of nh328_{—}5 indicates that it may contain some GAA/TIGGER repeat sequences. nh328_{—}5 protein was expressed in a COS cell expression system, and an expressed protein band of approximately 70 kDa was detected in membrane fractions using SDS polyacrylamide gel electrophoresis.

Clone “nm214_{—}3”

A polynucleotide of the present invention has been identified as clone “n214_{—}3”. nm214_{—}3 was isolated from a human adult blood (erythroleukemia TF-1) cDNA library using methods which are selective for cDNAs encoding secreted proteins (see U.S. Pat.

No.5,536,637), or was identified as encoding a secreted or transmembrane protein on the basis of computer analysis of the amino acid sequence of the encoded protein. nrn214_{—}3 is a full-length clone, including the entire coding sequence of a secreted protein (also referred to herein as “nrn214_{—}3 protein”).

The nucleotide sequence of nmn214_{—}3 as presently determined is reported in SEQ ID NO:95, and includes a poly(A) tail. What applicants presently believe to be the proper reading frame and the predicted amino acid sequence of the n-nL214_{—}3 protein corresponding to the foregoing nucleotide sequence is reported in SEQ ID NO:96.

The EcoRI/NotI restriction fragment obtainable from the deposit containing clone nm214_{—}3 should be approximately 1300 bp.

The nucleotide sequence disclosed herein for nm214_{—}3 was searched against the GenBank and GeneSeq nucleotide sequence databases using BLASTN/BLASTX and FASTA search protocols. nm214_{—}3 demonstrated at least some similarity with sequences identified as D10083 (Human RGH1 gene), D11078 (Human RGH2 gene), R68638 (yi06g11.s1 Homo sapiens cDNA clone 138500 3′), U88895 (Human endogenous retrovirus H Dl leader region/integrase-derived ORFI, ORF2, and putative envelope protein mRNA, complete cds), Z95327 (Human DNA sequence *** SEQUENCING IN PROGRESS from clone 347M6; HTGS phase 1), and Z97183 (Human DNA sequence *** SEQUENCING IN PROGRESS from clone ICB2046; HTGS phase 1). The predicted amino acid sequence disclosed herein for nm214_{—}3 was searched against the GenPept and GeneSeq amino acid sequence databases using the BLASTX search protocol. The predicted nm214_{—}3 protein demonstrated at least some similarity to sequences identified as U88895 (HERV-H integrase/envelope region [Homo sapiens]). Based upon sequence similarity, nm214_{—}3 proteins and each similar protein or peptide may share at least some activity. The nm214_{—}3 protein has a putative signal sequence at amino acids 13 to 25 of SEQ ID NO:96, with the mature protein starting at amino acid 26. The TopPredHI computer program predicts a potential transmembrane domain within the nm214_{—}3 protein sequence cewntered around amino acid 90 of SEQ ID NO:96. nrm214_{—}3 protein was expressed in a COS cell expression system, and an expressed protein band of approximately 13 kDa was detected in conditioned medium and membrane fractions using SDS polyacrylamide gel electrophoresis.

Clone “nn320_{—}2”

A polynucleotide of the present invention has been identified as clone “nn320_{—}2”. nn320_{—}2 was isolated from a human fetal kidney (293 cell line) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. nn320_{—}2 is a full-length clone, including the entire coding sequence of a secreted protein (also referred to herein as “nn320_{—}2 protein”).

The nucleotide sequence of nn320_{—}2 as presently determined is reported in SEQ ID NO:97, and includes a poly(A) tail. What applicants presently believe to be the proper reading frame and the predicted amino acid sequence of the nn320_{—}2 protein corresponding to the foregoing nucleotide sequence is reported in SEQ ID NO:98. Amino acids 4 to 16 of SEQ ID NO:98 are a predicted leader/signal sequence, with the predicted mature amino acid sequence beginning at amino acid 17. Due to the hydrophobic nature of the predicted leader/signal sequence, it is likely to act as a transmembrane domain should the predicted leader/signal sequence not be separated from the remainder of the nn320 2 protein.

The EcoRI/NotI restriction fragment obtainable from the deposit containing clone nn320_{—}2 should be approximately 2500 bp.

The nucleotide sequence disclosed herein for nn320_{—}2 was searched against the GenBank and GeneSeq nucleotide sequence databases using BLASTN/BLASTX and FASTA search protocols. nn320_{—}2 demonstrated at least some similarity with sequences identified as AA423969 (zv79h04.r1 Soares total fetus Nb2HF8 9w Homo sapiens cDNA clone 759895 5′) and AA423988 (zv79h04.s1 Soares total fetus Nb2HF8 9w Homo sapiens cDNA clone 759895 3′). The predicted amino acid sequence disclosed herein for nn320_{—}2 was searched against the GenPept and GeneSeq amino acid sequence databases using the BLASTX search protocol. The predicted nn320_{—}2 protein demonstrated at least some similarity to sequences identified as M60351 (filamentous hemagglutinin [Bordetella pertussis]) and R05041 (Filamentous haemagglutinin A). The predicted nn320_{—}2 protein also demonstrated similarity to a variety of proteases and enzyme precursors such as trypsinogen precursor. Based upon sequence similarity, nn320_{—}2 proteins and each similar protein or peptide may share at least some activity.

nn320_{—}2 protein was expressed in a COS cell expression system, and an expressed protein band of approximately 58 kDa was detected in conditioned medium and membrane fractions using SDS polyacrylamide gel electrophoresis.

Clone “pp392_{—}3”

A polynucleotide of the present invention has been identified as clone “pp392_{—}3”. pp392_{—}3 was isolated from a human adult blood (lymphoblastic leukemia MOLT-4) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. pp392_{—}3 is a full-length clone, including the entire coding sequence of a secreted protein (also referred to herein as “pp392_{—}3 protein”).

The nucleotide sequence of pp392_{—}3 as presently determined is reported in SEQ ID NO:99, and includes a poly(A) tail. What applicants presently believe to be the proper reading frame and the predicted amino acid sequence of the pp392_{—}3 protein corresponding to the foregoing nucleotide sequence is reported in SEQ ID NO:100.

The EcoRI/NotI restriction fragment obtainable from the deposit containing clone pp392 3 should be approximately 2100 bp. The nucleotide sequence disclosed herein for pp392_{—}3 was searched against the GenBank and GeneSeq nucleotide sequence databases using BLASTN/BLASTX and FASTA search protocols. pp392_{—}3 demonstrated at least some similarity with sequences identified as AA117686 (mo64c07.r1 Stratagene mouse heart (#937316) Mus musculus cDNA clone 558348 5′) and AL008726 (Human DNA sequence *** SEQUENCING IN PROGRESS from clone 337018; HTGS phase 1). Based upon sequence similarity, pp392_{—}3 proteins and each similar protein or peptide may share at least some activity. The pp392_{—}3 protein has a putative signal sequence at amino acids 196 to 208 of SEQ ID NO:100, with the mature protein starting at amino acid 209. The TopPredII computer program predicts three potential transmembrane domains within the pp392_{—}3 protein sequence centered around amino acids 20, 130, and 310 of SEQ ID NO: 100, respectively. The nucleotide sequence of pp392_{—}3 indicates that it may contain a CA repetitive element.

pp392_{—}3 protein was expressed in a COS cell expression system, and an expressed protein band of approximately 56 kDa was detected in membrane fractions using SDS polyacrylamide gel electrophoresis.

Clone “ya13_{—}1”

A polynucleotide of the present invention has been identified as clone “ya13_{—}1”. ya13_{—}1 was isolated from a human adult testes cDNA library and was identified as encoding a secreted or transmembrane protein on the basis of computer analysis of the amino acid sequence of the encoded protein. ya13_{—}1 is a full-length clone, including the entire coding sequence of a secreted protein (also referred to herein as “ya13_{—}1 protein”).

The nucleotide sequence of ya13_{—}1 as presently determined is reported in SEQ ID NO:101, and includes a poly(A) tail. What applicants presently believe to be the proper reading frame and the predicted amino acid sequence of the yal3_{—}1 protein corresponding to the foregoing nucleotide sequence is reported in SEQ ID NO:102. Amino acids 72 to 84 of SEQ ID NO:102 are a predicted leader/signal sequence, with the predicted mature amino acid sequence beginning at amino acid 85. Due to the hydrophobic nature of the predicted leader/signal sequence, it is likely to act as a transmembrane domain should the predicted leader/signal sequence not be separated from the remainder of the ya13_{—}1 protein.

The EcoRI/NotI restriction fragment obtainable from the deposit containing clone ya13_{—}1 should be approximately 750 bp.

The nucleotide sequence disclosed herein for ya13_{—}1 was searched against the GenBank and GeneSeq nucleotide sequence databases using BLASTN/BLASTX and FASTA search protocols. ya13_{—}1 demonstrated at least some similarity with sequences identified as AA190721 (zp88a07.r1 Stratagene HeLa cell s3 937216 Homo sapiens cDNA clone 627252 5′). Based upon sequence similarity, ya13_{—}1 proteins and each similar protein or peptide may share at least some activity.

Clone “yb37_{—}1”

A polynucleotide of the present invention has been identified as clone “yb37_{—}1”. yb37_{—}1 was isolated from a human fetal brain cDNA library and was identified as encoding a secreted or transmembrane protein on the basis of computer analysis of the amino acid sequence of the encoded protein. yb37_{—}1 is a full-length clone, including the entire coding sequence of a secreted protein (also referred to herein as “yb37_{—}1 protein”).

The nucleotide sequence of yb37_{—}1 as presently determined is reported in SEQ ID NO:103, and includes a poly(A) tail. What applicants presently believe to be the proper reading frame and the predicted amino acid sequence of the yb37_{—}1 protein corresponding to the foregoing nucleotide sequence is reported in SEQ ID NO:104. Amino acids 28 to 40 of SEQ ID NO:104 are a predicted leader/signal sequence, with the predicted mature amino acid sequence beginning at amino acid 41. Due to the hydrophobic nature of the predicted leader/signal sequence, it is likely to act as a transmembrane domain should the predicted leader/signal sequence not be separated from the remainder of the yb37_{—}1 protein. The TopPredll computer program predicts an additional potential transmembrane domain within the yb37_{—}1 protein sequence centered around amino acid 144 of SEQ ID NO:104.

Another possible reading frame and predicted amino acid sequence encoded by yb37_{—}1 is reported in SEQ ID NO:275; amino acids 49 to 61 of SEQ ID NO:275 are a predicted leader/signal sequence, with the predicted mature amino acid sequence beginning at amino acid 62. Due to the hydrophobic nature of this predicted leader/signal sequence, it is likely to act as a transmembrane domain should it not be separated from the remainder of the protein of SEQ ID NO:275.

The nucleotide sequence disclosed herein for yb37_{—}1 was searched against the GenBank and GeneSeq nucleotide sequence databases using BLASTN/BLASTX and FASTA search protocols. No hits were found in the database. The nucleotide sequence of yb37_{—}1 indicates that it may contain one or more A/TAAA repetitive elements.

yb37_{—}1 protein was expressed in a COS cell expression system, and an expressed protein band of approximately 33 kDa was detected in conditioned medium fractions using SDS polyacrylamide gel electrophoresis.

Clone “yb39_{—}1”

A polynucleotide of the present invention has been identified as clone “yb39_{—}1”. yb39_{—}1 was isolated from a human fetal brain cDNA library and was identified as encoding a secreted or transmembrane protein on the basis of computer analysis of the amino acid sequence of the encoded protein. yb39_{—}1 is a full-length clone, including the entire coding sequence of a secreted protein (also referred to herein as “yb39_{—}1 protein”).

The nucleotide sequence of yb39_{—}1 as presently determined is reported in SEQ ID NO:105, and includes a poly(A) tail. What applicants presently believe to be the proper reading frame and the predicted amino acid sequence of the yb39_{—}1 protein corresponding to the foregoing nucleotide sequence is reported in SEQ ID NO:106. Amino acids 21 to 33 of SEQ ID NO:106 are a predicted leader/signal sequence, with the predicted mature amnino acid sequence beginning at amino acid 34. Due to the hydrophobic nature of the predicted leader/signal sequence, it is likely to act as a transmembrane domain should the predicted leader/signal sequence not be separated from the remainder of the yb39_{—}1 protein.

The EcoRI/Notl restriction fragment obtainable from the deposit containing clone yb39_{—}1 should be approximately 825 bp.

The nucleotide sequence disclosed herein for yb39_{—}1 was searched against the GenBank and GeneSeq nucleotide sequence databases using BLASTN/BLASTX and FASTA search protocols. No hits were found in the database.

Clone “bd577_{—}1”

A polynucleotide of the present invention has been identified as clone “bd577_{—}1”. bd577_{—}1 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bd577_{—}1 is a full-length ;clone, including the entire coding sequence of a secreted protein (also referred to herein 3as “bd577_{—}1 protein”).

The nucleotide sequence of bd577_{—}1 as presently determined is reported in SEQ ID NO:107, and includes a poly(A) tail. What applicants presently believe to be the proper reading frame and the predicted amino acid sequence of the bd577_{—}1 protein corresponding to the foregoing nucleotide sequence is reported in SEQ ID NO:108. Amnino acids 42 to 54 of SEQ ID NO:108 are a predicted leader/signal sequence, with the predicted mature amino acid sequence beginning at amino acid 55. Due to the hydrophobic nature of the predicted leader/signal sequence, it is likely to act as a transmembrane domain should the predicted leader/signal sequence not be separated from the remainder of the bd577_{—}1 protein.

Another possible reading frame and predicted amino acid sequence encoded by base pairs 23 to 412 of bd577_{—}1 SEQ ID NO:107 is reported in SEQ ID NO:276; the amino acid sequence of SEQ ID NO:276 has a possible signal sequence from amino acids 57 to 69, with the predicted mature amino acid sequence beginning at amino acid 70. The open reading frames corresponding to SEQ ID NO:276 and SEQ ID NO:108 could be joined if a frameshift were introduced into the nucleotide sequence of SEQ ID NO:107.

The EcoRI/NotI restriction fragment obtainable from the deposit containing clone bd577_{—}1 should be approximately 1800 bp.

The nucleotide sequence disclosed herein for bd577_{—}1 was searched against the GenBank and GeneSeq nucleotide sequence databases using BLASTN/BLASTX and FASTA search protocols. bd577_{—}1 demonstrated at least some similarity with sequences identified as AA306618 (EST177563 Jurkat T-cells VI Homo sapiens cDNA 5′ end) and R20055 (yg39b06.r1 Homo sapiens cDNA clone 34805 5′). Based upon sequence similarity, bd577_{—}1 proteins and each similar protein or peptide may share at least some activity. The TopPredII computer program predicts two potential transmembrane domains within the bd577_{—}1 protein sequence centered around amino acids 42 and 230 of SEQ ID NO:108.

bd577_{—}1 protein was expressed in a COS cell expression system, and an expressed protein band of approximately 56 kDa was detected in membrane fractions using SDS polyacrylamide gel electrophoresis. Clone “by280_{—}3”

A polynucleotide of the present invention has been identified as clone “bv280_{—}3”. bv280_{—}3 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bv280_{—}3 is a full-length clone, including the entire coding sequence of a secreted protein (also referred to herein as “bv280_{—}3 protein”).

The nucleotide sequence of bv280_{—}3 as presently determined is reported in SEQ ID NO:109, and includes a poly(A) tail. What applicants presently believe to be the proper reading frame and the predicted amino acid sequence of the bv280_{—}3 protein corresponding to the foregoing nucleotide sequence is reported in SEQ ID NO:110. Amino acids 10 to 22 of SEQ ID NO:110 are a predicted leader/signal sequence, with the predicted mature amino acid sequence beginning at amino acid 23. Due to the hydrophobic nature of the predicted leader/signal sequence, it is likely to act as a transmembrane domain should the predicted leader/signal sequence not be separated from the remainder of the bv280_{—}3 protein.

The EcoRI/NotI restriction fragment obtainable from the deposit containing clone bv280_{—}3 should be approximately 1900 bp.

The nucleotide sequence disclosed herein for bv280_{—}3 was searched against the GenBank and GeneSeq nucleotide sequence databases using BLASTN/BLASTX and FASTA search protocols. bv28O_{—}3 demonstrated at least some similarity with sequences identified as AA095665 (15468.seq.F Fetal heart, Lambda ZAP Express Homo sapiens cDNA 5′), AA577430 (nm96g10.s1 NCI_CGAP_Co9 Homo sapiens cDNA clone IMAGE:1076130 similar to TR:G945383 G945383 CARBOXYPEPTIDASE), F06654 (H. sapiens partial cDNA sequence; clone c-1gal2), F08501 (H. sapiens partial cDNA), and H10119 (ym03f03.r1 Homo sapiens cDNA clone 46734 5′ similar to SP:A41612 A41612 VITELLOGENIC CARBOXYPEPTIDASE). The predicted amino acid sequence disclosed herein for bv280_{—}3 was searched against the GenPept and GeneSeq amino acid sequence databases using the BLASTX search protocol. The predicted bv280_{—}3 protein demonstrated at least some similarity to sequences identified as L46594 (carboxypeptidase [Aedes aegypti]) and R96737 (A. niger Bo-1 carboxypeptidase Y). Based upon sequence similarity, bv280_{—}3 proteins and each similar protein or peptide may share at least some activity. The bv280_{—}3 protein also has a serine carboxipeptidase active site motif (residues 195_{—}212). This motif is highly specific to serine carboxypeptidases and is not found in any other type of protein in the Swiss-Prot database. The bv280_{—}3 protein also has one copy of the crystallins beta and gamma ‘Greek key’ motif signature. The TopPredII computer program predicts another potential transmembrane domain within the bv280_{—}3 protein sequence centered around amino acid 110 of SEQ ID NO:110. bv280_{—}3 protein was expressed in a COS cell expression system, and an expressed protein band of approximately 61 kDa was detected in conditioned medium fractions using SDS polyacrylamide gel electrophoresis.

Clone “co315_{—}3”

A polynucleotide of the present invention has been identified as clone “co315_{—}3”. co315_{—}3 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. co315_{—}3 is a full-length clone, including the entire coding sequence of a secreted protein (also referred to herein as “co315_{—}3 protein”).

The nucleotide sequence of co315_{—}3 as presently determined is reported in SEQ ID NO:111, and includes a poly(A) tail. What applicants presently believe to be the proper reading frame and the predicted amino acid sequence of the co315 3 protein corresponding to the foregoing nucleotide sequence is reported in SEQ ID NO:112. Amino acids 51 to 63 of SEQ ID NO:112 are a predicted leader/signal sequence, with the predicted mature amino acid sequence beginning at amino acid 64. Due to the hydrophobic nature of the predicted leader/signal sequence, it is likely to act as a transmembrane domain should the predicted leader/signal sequence not be separated from the remainder of the co315_{—}3 protein.

The EcoRI/NotI restriction fragment obtainable from the deposit containing clone co315_{—}3 should be approximately 710 bp.

The nucleotide sequence disclosed herein for co315_{—}3 was searched against the GenBank and GeneSeq nucleotide sequence databases using BLASTN/BLASTX and FASTA search protocols. co315_{—}3 demonstrated at least some similarity with sequences identified as AA031371 (zk15e11.s1 Soares pregnant uterus NbHPU Homo sapiens cDNA clone 470636 3′), AA026051 (ze86aO7.s1 Soares fetal heart NbHH19W Homo sapiens), AA393961 (zt78b10.r1 Soares testis NHT Homo sapiens cDNA clone 728443 5′), AA481047 (aa29c06.s1 NCI_CGAP_GCB1 Homo sapiens cDNA clone IVIAGE:814666 3′), H46323 (yo15c05.r1 Homo sapiens cDNA clone 177992 5′), N23329 (yx78h09.s1 Homo sapiens cDNA clone 267905 3′), and R43942 (yg22f02.s1 Homo sapiens cDNA clone 33080 3′ similar to gb:M14648 VITRONECTIN RECEPTOR ALPHA SUBUNIT PRECURSOR (HUMAN)). Based upon sequence similarity, co315_{—}3 proteins and each similar protein or peptide may share at least some activity.

Clone “ij226_{—}6”

A polynucleotide of the present invention has been identified as clone “ij226_{—}6”. ij226_{—}6 was isolated from a human adult blood (peripheral blood mononuclear cells treated with granulocyte-colony stimulating factor in vivo) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ij226_{—}6 is a full-length clone, including the entire coding sequence of a secreted protein (also referred to herein as “ij226_{—}6 protein”).

The nucleotide sequence of ij226_{—}6 as presently determined is reported in SEQ ID NO:113, and includes a poly(A) tail. What applicants presently believe to be the proper reading frame and the predicted amino acid sequence of the ij226_{—}6 protein corresponding to the foregoing nucleotide sequence is reported in SEQ ID NO:114.

The EcoRI/NotI restriction fragment obtainable from the deposit containing clone ij226_{—}6 should be approximately 2300 bp.

The nucleotide sequence disclosed herein for ij226_{—}6 was searched against the GenBank and GeneSeq nucleotide sequence databases using BLASTN/BLASTX and FASTA search protocols. ij226_{—}6 demonstrated at least some similarity with sequences identified as AE000658 (Homo sapiens T-cell receptor alpha delta locus from bases 1 to 250529 (section 1 of 5) of the Complete Nucleotide Sequence), AF004231 (Homo sapiens monocyte/macrophage Ig-related receptor MIR-10 (MIR cl-10) MRNA, complete cds), G35352 (STS h14a108 5), H54023 (yq88h01.s1 Homo sapiens cDNA), H54181 (yq88h01.r1 Homo sapiens cDNA clone 202897 5′), T18551 (Human polycystic kidney disease normal PKD1 gene), and Z82206 (Human DNA sequence SEQUENCING IN PROGRESS from clone 370M22; HTGS phase 1). The predicted amino acid sequence disclosed herein for ij226_{—}6 was searched against the GenPept and GeneSeq amino acid sequence databases using the BLASTX search protocol. The predicted ij226_{—}6 protein demonstrated at least some similarity to sequences identified as M22334 (unknown protein [Homo sapiens]). Based upon sequence similarity, ij226_{—}6 proteins and each similar protein or peptide may share at least some activity. The TopPredHI computer program predicts two potential transmembrane domains within the ij226_{—}6 protein sequence centered around amino acids 37 and 62 of SEQ ID NO:114. The nucleotide sequence of ij226_{—}6 indicates that it may contain one or more of the following repetitive elements: Li, Alu, SVA.

Clone “nf443_{—}1”

A polynucleotide of the present invention has been identified as clone “nf443_{—}1”. nf443_{—}1 was isolated from a human adult brain (substantia nigra) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. nf443_{—}1 is a full-length clone, including the entire coding sequence of a secreted protein (also referred to herein as “nf443_{—}1 protein”).

The nucleotide sequence of nf443_{—}1 as presently determined is reported in SEQ ID NO:115, and includes a poly(A) tail. What applicants presently believe to be the proper reading frame and the predicted amino acid sequence of the nf443_{—}1 protein corresponding to the foregoing nucleotide sequence is reported in SEQ ID NO:116. Amino acids 21 to 43 of SEQ ID NO:116 are a possible leader/signal sequence, with the predicted mature amino acid sequence beginning at amino acid 44. Due to the hydrophobic nature of this possible leader/signal sequence, it is likely to act as a transmembrane domain should the leader/signal sequence not be separated from the remainder of the nf443_{—}1 protein.

The EcoRI/NotI restriction fragment obtainable from the deposit containing clone nf443_{—}1 should be approximately 3800 bp.

The nucleotide sequence disclosed herein for nf443_{—}1 was searched against the GenBank and GeneSeq nucleotide sequence databases using BLASTN/BLASTX and FASTA search protocols. nf443_{—}1 demonstrated at least some similarity with sequences identified as AA417092 (zuO7al2.s1 Soares testis NHT Homo sapiens cDNA clone 731134 3′), AA421511 (zu07a12.r1 Soares testis NHT Homo sapiens cDNA clone 731134 5′), T23707 (Human gene signature HUMGS05583), and U61233 (Bos taurus tubulin-folding cofactor D mRNA, complete cds). The predicted amino acid sequence disclosed herein for nf443_{—}1 was searched against the GenPept and GeneSeq amino acid sequence databases using the BLASTX search protocol. The predicted nf443_{—}1 protein demonstrated at least some similarity to sequences identified as U61233 (cofactor D [Bos taurus]). Based upon sequence similarity, nf443_{—}1 proteins and each similar protein or peptide may share at least some activity. The nucleotide sequence of nf443_{—}1 indicates that it may contain an Alu repetitive element.

nf443_{—}1 protein was expressed in a COS cell expression system, and an expressed protein band of approximately 10 kDa was detected in conditioned medium fractions using SDS polyacrylamide gel electrophoresis.

Clone “nt429_{—}1”

A polynucleotide of the present invention has been identified as clone “nt429_{—}1”. nt429_{—}1 was isolated from a human adult brain (corpus callosum) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. nt429_{—}1 is a full-length clone, including the entire coding sequence of a secreted protein (also referred to herein as “nt429_{—}1 protein”).

The nucleotide sequence of nt429_{—}1 as presently determined is reported in SEQ ID NO:117, and includes a poly(A) tail. What applicants presently believe to be the proper reading frame and the predicted amino acid sequence of the nt429_{—}1 protein corresponding to the foregoing nucleotide sequence is reported in SEQ ID NO:118. Another possible reading frame and predicted amino acid sequence, encoded by base pairs 399 to 731 of nt429_{—}1 SEQ ID NO:117, is reported in SEQ ID NO:277; the amino acid sequence of SEQ ID NO:277 is hydrophobic in nature near its carboxyl terminus. The overlapping open reading frames corresponding to SEQ ID NO:118 and SEQ ID NO:277 could be joined if a frameshift were introduced into the nucleotide sequence of SEQ ID NO:117.

The EcoRI/NotI restriction fragment obtainable from the deposit containing clone nt429_{—}1 should be approximnately 1800 bp.

The nucleotide sequence disclosed herein for nt429_{—}1 was searched against the GenBank and GeneSeq nucleotide sequence databases using BLASTN/BLASTX and FASTA search protocols. No significant hits were found in the database. The nucleotide sequence of nt429_{—}1 indicates that it may contain one or more of the following repetitive elements: Alu, SVA, A.

Clone “pe503_{—}1”

A polynucleotide of the present invention has been identified as clone “pe503_{—}1”. pe503_{—}1 was isolated from a human adult blood (chronic myelogenous leukemia K5) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. pe503_{—}1 is a full-length clone, including the entire coding sequence of a secreted protein (also referred to herein as “pe503_{—}1 protein”). The nucleotide sequence of pe503_{—}1 as presently determined is reported in SEQ ID NO:119, and includes a poly(A) tail. What applicants presently believe to be the proper reading frame and the predicted amino acid sequence of the pe503_{—}1 protein corresponding to the foregoing nucleotide sequence is reported in SEQ ID NO:120. Amino acids 79 to 91 of SEQ ID NO:120 are a predicted leader/signal sequence, with the predicted mature amino acid sequence beginning at amino acid 92. Due to the hydrophobic nature of the predicted leader/signal sequence, it is likely to act as a transmembrane domain should the predicted leader/signal sequence not be separated from the remainder of the pe503_{—}1 protein.

The EcoRI/NotI restriction fragment obtainable from the deposit containing clone pe503_{—}1 should be approximately 1300 bp.

The nucleotide sequence disclosed herein for pe503_{—}1 was searched against the GenBank and GeneSeq nucleotide sequence databases using BLASTN/BLASTX and FASTA search protocols. pe503_{—}1 demonstrated at least some similarity with sequences identified as AA298572 (EST114204 HSC172 cells I Homo sapiens cDNA 5′ end), AA595242 (no33a12.s1 NCI_CGAP_Pr23 Homo sapiens cDNA clone IMAGE:1102462), H60941 (yr14g06.r1 Homo sapiens cDNA clone 205306 5′), H75686 (yr77g08.r1 Homo sapiens cDNA clone 2113585′), and R61206 (yhO6d11.r1 Homo sapiens cDNA clone 42649 5′). Based upon sequence similarity, pe503_{—}1 proteins and each similar protein or peptide may share at least some activity. The TopPredII computer program predicts four potential transmembrane domains within the pe503_{—}1 protein sequence centered around amino acids 50, 84, 107, and 148 of SEQ ID NO:120, respectively.

pe503_{—}1 protein was expressed in a COS cell expression system, and an expressed protein band of approximately 19 kDa was detected in membrane fractions using SDS polyacrylamide gel electrophoresis.

Clone “pe834_{—}6”

A polynucleotide of the present invention has been identified as clone “pe834_{—}6”. pe834_{—}6 was isolated from a human adult blood (chronic myelogenous leukemia K5) cDNA library using methods which are selective for cDNAs encoding secreted proteins (see U.S. Pat. No.5,536,637), or was identified as encoding a secreted or transmembrane protein on the basis of computer analysis of the amino acid sequence of the encoded protein. pe834_{—}6 is a full-length clone, including the entire coding sequence of a secreted protein (also referred to herein as “pe834_{—}6 protein”).

The nucleotide sequence of pe834_{—}6 as presently determined is reported in SEQ ID NO:121, and includes a poly(A) tail. What applicants presently believe to be the proper reading frame and the predicted amino acid sequence of the pe834_{—}6 protein corresponding to the foregoing nucleotide sequence is reported in SEQ ID NO:122.

Another possible reading frame and predicted amino acid sequence, encoded by base pairs 414 to 725 of pe834_{—}6 SEQ ID NO:121, is reported in SEQ ID NO:278; the amino acid sequence of SEQ ID NO:278 is hydrophobic in nature near its carboxyl terminus. The overlapping open reading frames corresponding to SEQ ID NO:122 and SEQ ID NO:278 could be joined if a frameshift were introduced into the nucleotide sequence of SEQ ID NO:121.

The EcoRI/NotI restriction fragment obtainable from the deposit containing clone pe834_{—}6 should be approximately 1300 bp. The nucleotide sequence disclosed herein for pe834_{—}6 was searched against the GenBank and GeneSeq nucleotide sequence databases using BLASTN/BLASTX and FASTA search protocols. pe834_{—}6 demonstrated at least some similarity with sequences identified as AA054341 (z168f04.s1 Stratagene colon (#937204) Homo sapiens cDNA clone 509791 3′), N21462 (yx57c10.s1 Homo sapiens cDNA clone 265842 3′), N34010 (yx75g07.r1 Homo sapiens cDNA clone 267612 5′), and T90232 (ye15c09.r1 Homo sapiens cDNA clone 1178085′). Based upon sequence similarity, pe834_{—}6 proteins and each similar protein or peptide may share at least some activity.

pe834_{—}6 protein was expressed in a COS cell expression system, and an expressed protein band of approximately 17 kDa was detected in membrane fractions using SDS polyacrylamide gel electrophoresis.

Clone “ya10_{—}1”

A polynucleotide of the present invention has been identified as clone “ya10_{—}1”. ya10_{—}1 was isolated from a human adult testes cDNA library and was identified as encoding a secreted or transmembrane protein on the basis of computer analysis of the amino acid sequence of the encoded protein. Ya10_{—}1 is a full-length clone, including the entire coding sequence of a secreted protein (also referred to herein as “ya10_{—}1 protein”).

The nucleotide sequence of ya10_{—}1 as presently determined is reported in SEQ ID NO:123, and includes a poly(A) tail. What applicants presently believe to be the proper reading frame and the predicted amnino acid sequence of the ya10_{—}1 protein corresponding to the foregoing nucleotide sequence is reported in SEQ ID NO:124.

Amino acids 6 to 18 of SEQ ID NO:124 are a predicted leader/signal sequence, with the predicted mature amino acid sequence beginning at amino acid 19. Due to the hydrophobic nature of the predicted leader/signal sequence, it is likely to act as a transmembrane domain should the predicted leader/signal sequence not be separated from the remainder of the ya10_{—}1 protein.

The EcoRI/NotI restriction fragment obtainable from the deposit containing clone ya10_{—}1 should be approximately 800 bp.

The nucleotide sequence disclosed herein for ya10_{—}1 was searched against the GenBank and GeneSeq nucleotide sequence databases using BLASTN/BLASTX and FASTA search protocols. No clearly significant hits were found in these databases. BLASTX analysis of the ya10_{—}1 protein sequence revealed some amino acid sequence similarity to cystatins (cysteine protease inhibitors) of various species. Based upon this sequence similarity, ya10_{—}1 proteins and each similar protein or peptide may share at least some activity.

Clone “yb40_{—}1”

A polynucleotide of the present invention has been identified as clone “yb40_{—}1”. yb40_{—}1 was isolated from a human fetal brain cDNA library and was identified as encoding a secreted or transmembrane protein on the basis of computer analysis of the amino acid sequence of the encoded protein. yb40_{—}1 is a full-length clone, including the entire coding sequence of a secreted protein (also referred to herein as “yb40_{—}1 protein”). The nucleotide sequence of yb40_{—}1 as presently determined is reported in SEQ ID NO: 125, and includes a poly(A) tail. What applicants presently believe to be the proper reading frame and the predicted amino acid sequence of the yb40_{—}1 protein corresponding to the foregoing nucleotide sequence is reported in SEQ ID NO:126. Amino acids 29 to 41 of SEQ ID NO:126 are a possible leader/signal sequence, with the predicted mature amino acid sequence beginning at amino acid 42. Due to the hydrophobic nature of this possible leader/signal sequence, it could act as a transmembrane domain should it not be separated from the remainder of the yb40_{—}1 protein.

The EcoRI/NotI restriction fragment obtainable from the deposit containing clone yb40_{—}1 should be approximately 1700 bp.

The nucleotide sequence disclosed herein for yb40_{—}1 was searched against the GenBank and GeneSeq nucleotide sequence databases using BLASTN/BLASTX and FASTA search protocols. yb40_{—}1 demonstrated at least some similarity with sequences identified as AA595189 (no32f03.s1 NCI_CGAP_Pr23 Homo sapiens cDNA clone IMAGE:1102397), R74575 (yi58d04.r1 Homo sapiens cDNA clone 143431 5′), and T25773 (Human gene signature HUMGS08001). Based upon sequence similarity, yb40_{—}1 proteins and each similar protein or peptide may share at least some activity.

Clone “cs756_{—}2”

A polynucleotide of the present invention has been identified as clone “cs756_{—}2”. cs756_{—}2 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cs756_{—}2 is a full-length clone, including the entire coding sequence of a secreted protein (also referred to herein as “cs756 2 protein”).

The nucleotide sequence of cs756_{—}2 as presently determined is reported in SEQ ID NO:127, and includes a poly(A) tail. What applicants presently believe to be the proper reading frame and the predicted amino acid sequence of the cs756 2 protein corresponding to the foregoing nucleotide sequence is reported in SEQ ID NO:128. Amino acids 211 to 223 of SEQ ID NO:128 are a predicted leader/signal sequence, with the predicted mature amino acid sequence beginning at amino acid 224. Due to the hydrophobic nature of the predicted leader/signal sequence, it is likely to act as a transmembrane domain should the predicted leader/signal sequence not be separated from the remainder of the cs756_{—}2 protein. The TopPredII computer program predicts a potential transmembrane domain within the cs756_{—}2 protein sequence of SEQ ID NO:128, centered around amino acid 15 of SEQ ID NO: 128; amino acids 2 to 14 of SEQ ID NO:128 are also a possible leader/signal sequence, with the predicted mature amino acid sequence in that case beginning at amino acid 15.

Another possible cs756_{—}2 reading frame and predicted amino acid sequence, encoded by base pairs 385 to 825 of SEQ ID NO:127, is reported in SEQ ID NO:279; the TopPredII computer program predicts a potential transmembrane domain centered around amino acid 100 of SEQ ID NO:279. The open reading frames corresponding to SEQ ID NO:279 and SEQ ID NO:128 could be joined if a frameshift were introduced into the nucleotide sequence of SEQ ID NO:127.

The EcoRI/NotI restriction fragment obtainable from the deposit containing clone cs756_{—}2 should be approximately 3000 bp.

The nucleotide sequence disclosed herein for cs756_{—}2 was searched against the GenBank and GeneSeq nucleotide sequence databases using BLASTN/BLASTX and FASTA search protocols. cs756_{—}2 demonstrated at least some similarity with sequences identified as AA398077 (zt58c03.s1 Soares testis NHT Homo sapiens cDNA clone 726532 3′), AA541286 (nf97e03.s1 NCI_CGAP_Co3 Homo sapiens cDNA clone IMAGE:927868), W28620 (49c2 Human retina cDNA randomly primed sublibrary Homo sapiens cDNA), and W47601 (zc35g08.r1 Soares senescent fibroblasts NbHSF Homo sapiens cDNA clone 324350 5′). The predicted amino acid sequence disclosed herein for SEQ ID NO:279 was searched against the GenPept and GeneSeq amino acid sequence databases using the BLASTX search protocol. The predicted SEQ ID NO:279 protein demonstrated at least some similarity to sequences identified as L76938 (Werner syndrome gene, complete cds [Homo sapiens]). “Wemer's syndrome (WS) is an inherited disease with clinical symptoms resembling premature aging . . . [the] predicted protein is 1432 amino acids in length and shows significant similarity to DNA helicases” (Yu et al., 1996, Science 272(5259):258_{—}262, which is incorporated by reference herein). Based upon sequence similarity, cs756_{—}2 proteins and each similar protein or peptide may share at least some activity. The nucleotide sequence of cs756_{—}2 indicates that it may contain one or more of the following repetitive elements: MIR, MER.

Clone “ew150_{—}1”

A polynucleotide of the present invention has been identified as clone “ew15O_{—}1”. ew150_{—}1 was isolated from a human adult placenta cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ew150_{—}1 is a full-length clone, including the entire coding sequence of a secreted protein (also referred to herein as “ew150_{—}1 protein”).

The nucleotide sequence of ew150_{—}1 as presently determined is reported in SEQ ID NO:129, and includes a poly(A) tail. What applicants presently believe to be the proper reading frame and the predicted amino acid sequence of the ew150_{—}1 protein corresponding to the foregoing nucleotide sequence is reported in SEQ ID NO:130. Amino acids 26 to 38 of SEQ ID NO:130 are a predicted leader/signal sequence, with the predicted mature amino acid sequence beginning at amino acid 39. Due to the hydrophobic nature of the predicted leader/signal sequence, it is likely to act as a transmembrane domain should the predicted leader/signal sequence not be separated from the remainder of the ew150_{—}1 protein.

The EcoRI/NotI restriction fragment obtainable from the deposit containing clone ew150_{—}1 should be approximately 2000 bp.

The nucleotide sequence disclosed herein for ew150_{—}1 was searched against the GenBank and GeneSeq nucleotide sequence databases using BLASTN/BLASTX and FASTA search protocols. ew150_{—}1 demonstrated at least some similarity with sequences identified as AA563938 (nk23b12.s1 NCI_CGAP_Coll Homo sapiens cDNA clone IMAGE 1014335), D63209 (Human placenta cDNA 5′-end GEN-506F01), M90423 (Bacteriphage US3 lytic-enzyme), W23461 (zb33c01.r1 Soares parathyroid tumor NbHPA Homo sapiens cDNA clone 305376 5′), and Z56916 (H.sapiens CpG DNA, clone 153b7, forward read cpg153b7.ft1a). In the region around position 1514 of SEQ ID NO:129, ew150_{—}1 also demonstrated at least some similarity with sequences encoding a mitochondrial energy-transfer proteins signature motif which is found in mitochondrial and other membrane proteins. Based upon sequence similarity, ewl501 proteins and each similar protein or peptide may share at least some activity. The TopPrediH computer program predicts ten potential transmembrane domains within the ew150_{—}1 protein sequence, which are centered around amino acids 70,106,133,200,314,349,387, 457,504, and 527 of SEQ ID NO:130, respectively.

Clone “gg894_{—}13”

A polynucleotide of the present invention has been identified as clone “gg894_{—}13”. gg894_{—}13 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. gg894_{—}13 is a full-length clone, including the entire coding sequence of a secreted protein (also referred to herein as “gg894 13 protein”).

The nucleotide sequence of gg894_{—}13 as presently determined is reported in SEQ ID NO:131, and includes a poly(A) tail. What applicants presently believe to be the proper reading frame and the predicted amino acid sequence of the gg894_{—}13 protein corresponding to the foregoing nucleotide sequence is reported in SEQ ID NO:132. Amino acids 41 to 53 of SEQ ID NO:132 are a predicted leader/signal sequence, with the predicted mature amino acid sequence beginning at amino acid 54. Due to the hydrophobic nature of the predicted leader/signal sequence, it is likely to act as a transmembrane domain should the predicted leader/signal sequence not be separated from the remainder of the gg894_{—}13 protein. Another possible gg894_{—}13 reading frame and predicted amino acid sequence, encoded by base pairs 602 to 1129 of SEQ ID NO:131, is reported in SEQ ID NO:280. The open reading frames corresponding to SEQ ID NO:280 and SEQ ID NO:132 could be joined if a frameshift were introduced into the nucleotide sequence of SEQ ID NO:131.

The EcoRI/NotI restriction fragment obtainable from the deposit containing clone gg894_{—}13 should be approximately 2400 bp.

The nucleotide sequence disclosed herein for gg894_{—}13 was searched against the GenBank and GeneSeq nucleotide sequence databases using BLASTN/BLASTX and FASTA search protocols. gg894_{—}13 demonstrated at least some similarity with sequences identified as H57424 (yr13a10.s1 Homo sapiens cDNA clone 205146 3′), T23885 (Human gene signature HUMGS05820), and W80358 (zh49aO7.s1 Soares fetal liver spleen 1NFLS S1 Homo sapiens cDNA clone 415380 3′). Based upon sequence similarity, gg894_{—}13 proteins and each similar protein or peptide may share at least some activity. The TopPredII computer program predicts a potential transmembrane domain within the gg894_{—}13 protein sequence centered around amino acid 115 of SEQ ID NO:132. The nucleotide sequence of gg894_{—}13 indicates that it may contain a RBMI repetitive element.

Clone “it217_{—}2”

A polynucleotide of the present invention has been identified as clone “it217_{—}2”. it217_{—}2 was isolated from a human adult brain (thalamus) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. it217_{—}2 is a full-length clone, including the entire coding sequence of a secreted protein (also referred to herein as “it217_{—}2 protein”).

The nucleotide sequence of it217_{—}2 as presently determined is reported in SEQ ID NO:133, and includes a poly(A) tail. What applicants presently believe to be the proper reading frame and the predicted amino acid sequence of the it217_{—}2 protein corresponding to the foregoing nucleotide sequence is reported in SEQ ID NO:134. Another possible it217_{—}2 reading frame and predicted amino acid sequence, encoded by base pairs 45 to 311 of SEQ ID NO:133, is reported in SEQ ID NO:281. Amino acids 36 to 48 of SEQ ID NO:281 are a predicted leader/signal sequence, with the predicted mature amino acid sequence beginning at amino acid 49. Due to the hydrophobic nature of the predicted leader/signal sequence, it is likely to act as a transmembrane domain should the predicted leader/signal sequence not be separated from the remainder of the it217_{—}2 protein. The open reading frames corresponding to SEQ ID NO:281 and SEQ ID NO:134 could be joined if at least one frameshift were introduced into the nucleotide sequence of SEQ ID NO:133.

The EcoRI/NotI restriction fragment obtainable from the deposit containing clone it217_{—}2 should be approximately 2250 bp.

The nucleotide sequence disclosed herein for it217_{—}2 was searched against the GenBank and GeneSeq nucleotide sequence databases using BLASTN/BLASTX and FASTA search protocols. it217_{—}2 demonstrated at least some similarity with sequences identified as AA242969 (zr65h09.r1 Soares NhHMPu S1 Homo sapiens cDNA clone 668321 5′ similar to SW SCC2_HUMAN P48594 SQUAMOUS CELL CARCINOMA ANTIGEN 2 ;contains Alu repetitive element), B44876 (HS-1060-A1-G06-MR.abi CIT Human Genomic Sperm Library C Homo sapien genomic clone Plate CT 782 Col 11 Row M), H82168 (yv78d08.r1 Homo sapiens cDNA clone), S66896 (squamous cell carcinoma antigen), U19556 (Human squamous cell carcinoma antigen 1 (SCCA1) mRNA, complete cds), U19557 (Human squamous cell carcinoma antigen 2 (SCCA2) mRNA, complete cds), and U35459 (Human bomapin mRNA, complete cds). The predicted amino acid sequence disclosed herein for it217_{—}2 was searched against the GenPept and GeneSeq amino acid sequence databases using the BLASTX search protocol. The predicted it217_{—}2 protein demonstrated at least some similarity to sequences identified as L40377 (cytoplasmic antiproteinase 2 [Homo sapiens]), M34352 (ovalbumin [Gallus gallus]), M91161 (serpin [Equus caballus]), R25276 (SCC antigen), R48379 (Human megakaryocyte differentiation factor), S66896 (squamous cell carcinoma antigen, SCC antigen serine protease inhibitor [human, Peptide, 390 aa] [Homo sapiensl), Ul19568 (squamous cell carcinoma antigen [Homo sapiens]), and U19576 (squamous cell carcinoma antigen [Homo sapiens]). Human bomapin may play a role in the regulation of protease activities during hematopoiesis (Riewald et al., 1995, J. Biol. Chem. 270: 26754, which is incorporated by reference herein). Serpins are SERine Proteinase JNltbitors and are considered extracellular in localization. Human squamous cell carcinoma antigen (SSCA) is a member of the serpin family of proteinase inhibitors, purified from sera of cancer patients. Based upon sequence similarity, it217_{—}2 proteins and each similar protein or peptide may share at least some activity.

Clone “ml235_{—}2”

A polynucleotide of the present invention has been identified as clone “ml235_{—}2”. ml235_{—}2 was isolated from a human adult brain (caudate nucleus) cDNA library using methods which are selective for cDNAs encoding secreted proteins (see U.S. Pat. No. 35,536,637), or was identified as encoding a secreted or transmembrane protein on the basis of computer analysis of the amino acid sequence of the encoded protein. ml235_{—}2 is a full-length clone, including the entire coding sequence of a secreted protein (also referred to herein as “ml235 2 protein”).

The nucleotide sequence of mnl235_{—}2 as presently determined is reported in SEQ ID NO:135, and includes a poly(A) tail. What applicants presently believe to be the proper reading frame and the predicted amino acid sequence of the mnl235 2 protein corresponding to the foregoing nucleotide sequence is reported in SEQ ID NO:136. Amino acids 3 to 15 of SEQ ID NO:136 are a predicted leader/signal sequence, with the predicted mature amino acid sequence beginning at amino acid 16. Due to the hydrophobic nature of the predicted leader/signal sequence, it is likely to act as a transmembrane domain should the predicted leader/signal sequence not be separated from the remainder of the ml235_{—}2 protein.

The EcoRI/NotI restriction fragment obtainable from the deposit containing clone mnl235_{—}2 should be approximately 1400 bp.

The nucleotide sequence disclosed herein for mi235_{—}2 was searched against the GenBank and GeneSeq nucleotide sequence databases using BLASTN/BLASTX and FASTA search protocols. ml235_{—}2 demonstrated at least some similarity with sequences identified as AA160887 (zo79b05.s1 Stratagene pancreas (#937208) Homo sapiens cDNA clone 593073 3′), R14349 (yf79f12.r1 Homo sapiens cDNA clone 28451 5′), and R54256 (yg74f07.r1 Homo sapiens cDNA clone 39059 5′). Based upon sequence similarity, ml235_{—}2 proteins and each similar protein or peptide may share at least some activity. The TopPredII computer program predicts a potential transmembrane domain within the ml235_{—}2 protein sequence centered around amino acid 25 of SEQ ID NO:136.

Clone “mt24_{—}2”

A polynucleotide of the present invention has been identified as clone “mt24_{—}2”. mt24_{—}2 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. mt24_{—}2 is a full-length clone, including the entire coding sequence of a secreted protein (also referred to herein as =“mt24_{—}2 protein”).

The nucleotide sequence of mt24 2 as presently determined is reported in SEQ ID NO:137, and includes a poly(A) tail. What applicants presently believe to be the proper reading frame and the predicted amino acid sequence of the mt24_{—}2 protein corresponding to the foregoing nucleotide sequence is reported in SEQ ID NO:138. Amino acids 30 to 42 of SEQ ID NO:138 are a predicted leader/signal sequence, with the predicted mature amino acid sequence beginning at amino acid 43. Due to the hydrophobic nature of the predicted leader/signal sequence, it is likely to act as a transmembrane domain should the predicted leader/signal sequence not be separated from the remainder of the mt24 2 protein.

The EcoRI/NotI restriction fragment obtainable from the deposit containing clone mt24_{—}2 should be approximately 1400 bp. The nucleotide sequence disclosed herein for mt24_{—}2 was searched against the GenBank and GeneSeq nucleotide sequence databases using BLASTN/BLASTX and FASTA search protocols. mt24_{—}2 demonstrated at least some similarity with sequences identified as AA062589 (zf68f04.r1 Soares pineal gland N3HPG Homo sapiens cDNA clone 382111 5′) and T19332 (b08016t Testis 1 Homo sapiens cDNA clone bO8016 5′ end). Based upon sequence similarity, mt24_{—}2 proteins and each similar protein or peptide may share at least some activity. The TopPredII computer program predicts four potential transmembrane domains within the mt24_{—}2 protein sequence centered around amino acids 38, 153, 167, and 232 of SEQ ID NO:138, respectively.

Clone “pe584_{—}2”

A polynucleotide of the present invention has been identified as clone “pe584_{—}2”. pe584 2 was isolated from a human adult blood (chronic myelogenous leukemia K5) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. pe584_{—}2 is a full-length clone, including the entire coding sequence of a secreted protein (also referred to herein as “pe584_{—}2 protein”).

The nucleotide sequence of pe584_{—}2 as presently determined is reported in SEQ ID NO:139, and includes a poly(A) tail. What applicants presently believe to be the proper reading frame and the predicted amino acid sequence of the pe584_{—}2 protein corresponding to the foregoing nucleotide sequence is reported in SEQ ID NO:140. Amino acids 27 to 39 of SEQ ID NO:140 are a predicted leader/signal sequence, with the predicted mature amino acid sequence beginning at amino acid 40. Due to the hydrophobic nature of the predicted leader/signal sequence, it is likely to act as a transmembrane domain should the predicted leader/signal sequence not be separated from the remainder of the pe584_{—}2 protein.

The EcoRI/NotI restriction fragment obtainable from the deposit containing clone pe584_{—}2 should be approximately 3000 bp. The nucleotide sequence disclosed herein for pe584_{—}2 was searched against the GenBank and GeneSeq nucleotide sequence databases using BLASTN/BLASTX and FASTA search protocols. pe584_{—}2 demonstrated at least some similarity with sequences identified as AA303149 (EST13039 Uterus tumor I), AA405004 (zt06e03.s1 NCI_CGAP_GCB1 Homo sapiens cDNA clone IMAGE 712348 3′), AA481230 (aa34g01.r1 NCI_CGAP_GCB1 Homo sapiens cDNA clone 815184 5′ similar to SW TCR2_ECOLI P02981 TETRACYCLINE RESISTANCE PROTEIN), D88315 (Mouse mRNA for tetracycline transporter-like protein, complete cds), and T10077 (seql295 Homo sapiens cDNA clone b4HB3MA-COT8-HAP-Ft109 5′). The predicted amino acid sequence disclosed herein for pe584_{—}2 was searched against the GenPept and GeneSeq amino acid sequence databases using the BLASTX search protocol. The predicted pe584_{—}2 protein demonstrated at least some similarity to sequences identified as D88315 (tetracycline transporter-like protein [Mus musculus]). Mouse tetracycline transporter-like protein is a sugar transporter (Matsuo et aZ., 1997, Biochem. Biophys. Res. Comm. 238: 126_{—}192, which is incorporated by reference herein). Based upon sequence similarity, pe584_{—}2 proteins and each similar protein or peptide may share at least some activity. The TopPredII computer program predicts eleven potential transmembrane domains within the pe584_{—}2 protein sequence, which are centered around amino acids 32, 55, 78, 114, 142, 196, 235, 264,287, 332, and 375 of SEQ ID NO:140, respectively.

Clone “pj323_{—}2”

A polynucleotide of the present invention has been identified as clone “pj323_{—}2”. pj323_{—}2 was isolated from a human fetal carcinoma (NTD2 cells treated with retinoic acid for 23 days) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. pj323_{—}2 is a full-length clone, including the entire coding sequence of a secreted protein (also referred to herein as “pj323 2 protein”). The nucleotide sequence of pj323 2 as presently determined is reported in SEQ ID NO:141, and includes a poly(A) tail. What applicants presently believe to be the proper reading frame and the predicted amino acid sequence of the pj323 2 protein corresponding to the foregoing nucleotide sequence is reported in SEQ ID NO:142. Amino acids 150 to 162 of SEQ ID NO:142 are a predicted leader/signal sequence, with the predicted mature amino acid sequence beginning at amino acid 163. Due to the hydrophobic nature of the predicted leader/signal sequence, it is likely to act as a transmembrane domain should the predicted leader/signal sequence not be separated from the remainder of the pj323_{—}2 protein.

The EcoRI/NotI restriction fragment obtainable from the deposit containing clone pj323_{—}2 should be approximately 2500 bp.

The nucleotide sequence disclosed herein for pj323_{—}2 was searched against the GenBank and GeneSeq nucleotide sequence databases using BLASTN/BLASTX and FASTA search protocols. pj323_{—}2 demonstrated at least some similarity with sequences identified as AA160454 (zo74g05.r1 Stratagene pancreas (#937208) Homo sapiens cDNA clone 592664 5′), AA398257 (zt60a08.s1 Soares testis NHT Homo sapiens cDNA clone 726710 3′), and T47284 (yb64g11.s1 Homo sapiens cDNA clone 76004 3′). The predicted amino acid sequence disclosed herein for pj323_{—}2 was searched against the Gen~ept and GeneSeq amino acid sequence databases using the BLASTX search protocol. The predicted pj323_{—}2 protein demonstrated at least some similarity to human integral nuclear envelope protein, lamnin B receptors from several species, and sterol reductases from several species. Lamin B receptors have hydrophobic carboxy terminal portions and hydrophilic amino terminal portions. Antibodies to lamin B receptors have been found in patients with primary biliary cirrhosis. Sterol reductases demonstrate sequence similarity to the hydrophobic portions of lamin B receptors. Based upon sequence similarity, pj323_{—}2 proteins and each similar protein or peptide may share at least some activity. The TopPredIU computer program predicts six potential transmembrane domains within the pj323_{—}2 protein sequence, which are centered around amino acids 47, 106, 164, 187, 341, and 432 of SEQ ID NO:142, respectively.

pj323_{—}2 protein was expressed in a COS cell expression system, and an expressed protein band of approximately 46 kDa was detected in membrane fractions using SDS polyacrylamide gel electrophoresis.

Clone “yb24_{—}1”

A polynucleotide of the present invention has been identified as clone “yb24_{—}1”. yb24_{—}1 was isolated from a human fetal brain cDNA library and was identified as encoding a secreted or transmembrane protein on the basis of computer analysis of the amino acid sequence of the encoded protein. yb24_{—}1 is a full-length clone, including the entire coding sequence of a secreted protein (also referred to herein as “yb24_{—}1 protein”).

The nucleotide sequence of yb24_{—}1 as presently determined is reported in SEQ ID NO:143, and includes a poly(A) tail. What applicants presently believe to be the proper reading frame and the predicted amino acid sequence of the yb24_{—}1 protein corresponding to the foregoing nucleotide sequence is reported in SEQ ID NO:144. Amino acids 25 to 37 of SEQ ID NO:144 are a predicted leader/signal sequence, with the predicted mature amino acid sequence beginning at amino acid 38. Due to the hydrophobic nature of the predicted leader/signal sequence, it is likely to act as a transmembrane domain should the predicted leader/signal sequence not be separated from the remainder of the yb24_{—}1 protein.

The EcoRI/NotI restriction fragment obtainable from the deposit containing clone yb24_{—}1 should be approximately 1700 bp.

The nucleotide sequence disclosed herein for yb24_{—}1 was searched against the GenBank and GeneSeq nucleotide sequence databases using BLASTN/BLASTX and FASTA search protocols. yb24_{—}1 demonstrated at least some similarity with sequences identified as AA149807 (zl47c09.s1 Soares pregnant uterus NbHPU Homo sapiens cDNA clone 505072 3′) and AB003515 (Rat mRNA for GEF-2, complete cds). Based upon sequence similarity, yb24_{—}1 proteins and each similar protein or peptide may share at least some activity.

Clone “yb44_{—}1”

A polynucleotide of the present invention has been identified as clone “yb44_{—}1”. yb44_{—}1 was isolated from a human fetal brain cDNA library and was identified as encoding a secreted or transmembrane protein on the basis of computer analysis of the amino acid sequence of the encoded protein. yb44_{—}1 is a full-length clone, including the entire coding sequence of a secreted protein (also referred to herein as “yb44_{—}1 protein”).

The nucleotide sequence of yb44_{—}1 as presently determined is reported in SEQ ID NO:145, and includes a poly(A) tail. What applicants presently believe to be the proper reading frame and the predicted amino acid sequence of the yb44_{—}1 protein corresponding to the foregoing nucleotide sequence is reported in SEQ ID NO:146. Amino acids 10 to 22 of SEQ ID NO:146 are a predicted leader/signal sequence, with the predicted mature amino acid sequence beginning at amino acid 23. Due to the hydrophobic nature of the predicted leader/signal sequence, it is likely to act as a transmembrane domain should the predicted leader/signal sequence not be separated from the remainder of the yb44_protein.

The EcoRI/NotI restriction fragment obtainable from the deposit containing clone yb44_{—}1 should be approximately 2000 bp.

The nucleotide sequence disclosed herein for yb44_{—}1 was searched against the GenBank and GeneSeq nucleotide sequence databases using BLASTN/BLASTX and FASTA search protocols. yb44_{—}1 demonstrated at least some similarity with sequences identified as AC000016 (*** SEQUENCING IN PROGRESS *** EPM1/APECED region of chromosome 21, BAC clone B4P3; H-TGS phase 1, 10 unordered pieces). The predicted amino acid sequence disclosed herein for yb44_{—}1 was searched against the GenPept and GeneSeq amino acid sequence databases using the BLASTX search protocol. The predicted yb44_{—}1 protein demonstrated at least some similarity to sequences identified as R72377 (Human auxillary cytochrome P450 species 2D6 variant 2 protein) and U44753 (cytochrome P450 [Drosophila melanogaster]). Based upon sequence similarity, yb44_{—}1 proteins and each similar protein or peptide may share at least some activity. The TopPredil computerprogrampredicts three additional potential transmembrane domains within the yb44_{—}1 protein sequence, which are centered around amino acids 82,128, and 361 of SEQ ID NO:146, respectively. The nucleotide sequence of yb44_{—}1 indicates that it may contain one or more of the following repetitive elements: Alu, AT, TATACA, MER44A, TACA.

Clone “bn69_{—}15”

A polynucleotide of the present invention has been identified as clone “bn69_{—}15”. bn69_{—}15 was isolated from a human adult placenta cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bn69_{—}15 is a full-length clone, including the entire coding sequence of a secreted protein (also referred to herein as “bn69 15 protein”).

The nucleotide sequence of bn69_{—}15 as presently determined is reported in SEQ ID NO:147, and includes a poly(A) tail. What applicants presently believe to be the proper reading frame and the predicted amino acid sequence of the bn69_{—}15 protein corresponding to the foregoing nucleotide sequence is reported in SEQ ID NO:148. Amino acids 47 to 59 of SEQ ID NO:148 are a predicted leader/signal sequence, with the predicted mature amino acid sequence beginning at amino acid 60. Due to the hydrophobic nature of the predicted leader/signal sequence, it is likely to act as a transmembrane domain should the predicted leader/signal sequence not be separated from the remainder of the bn69_{—}15 protein. Another potential bn69_{—}15 reading frame and predicted amino acid sequence is encoded by basepairs 1008 to 1352 of SEQ ID NO:147 and is reported in SEQ ID NO:282.

The EcoRI/NotI restriction fragment obtainable from the deposit containing clone bn69_{—}15 should be approximately 2800 bp.

The nucleotide sequence disclosed herein for bn69_{—}15 was searched against the GenBank and GeneSeq nucleotide sequence databases using BLASTN/BLASTX and FASTA search protocols. bn69_{—}15 demonstrated at least some similarity with sequences identified as H80692 (yv01b10.r1 Homo sapiens cDNA clone 241435 5′), T64701 (yc48d02.r1 Homo sapiens cDNA clone 83907 5′), and W21368 (zb59c01.r1 Soares fetal lung NbHL19W Homo sapiens cDNA clone 307872 5′ similar to gb:M83186 CYTOCHROME C OXIDASE POLYPEPTIDE VIIA-HEART PRECURSOR (HUMAN)). Based upon sequence similarity, bn69_{—}15 proteins and each similar protein or peptide may share at least some activity. The TopPredII computer program predicts an additional potential transmembrane domain within the bn69_{—}15 protein sequence centered around amino acid 32 of SEQ ID NO:148.

Clone “cb110_{—}1”

A polynucleotide of the present invention has been identified as clone “cb110_{—}1”. cb110_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. Cb110_{—}1 is a full-length clone, including the entire coding sequence of a secreted protein (also referred to herein as “cb110_{—}1 protein”).

The nucleotide sequence of cb110_{—}1 as presently determined is reported in SEQ ID NO:149, and includes a poly(A) tail. What applicants presently believe to be the proper reading frame and the predicted amino acid sequence of the cb110_{—}1 protein corresponding to the foregoing nucleotide sequence is reported in SEQ ID NO:150. Amino acids 36 to 48 of SEQ ID NO:150 are a predicted leader/signal sequence, with the predicted mature amino acid sequence beginning at amino acid 49. Due to the hydrophobic nature of the predicted leader/signal sequence, it is likely to act as a transmembrane domain should the predicted leader/signal sequence not be separated from the remainder of the cb110_{—}1 protein.

The EcoRI/NotI restriction fragment obtainable from the deposit containing clone cb110_{—}1 should be approximately 900 bp.

The nucleotide sequence disclosed herein for cbllO_{—}1 was searched against the GenBank and GeneSeq nucleotide sequence databases using BLASTN/BLASTX and FASTA search protocols. Cb110_{—}1 demonstrated at least some similarity with sequences identified as AC001083 (Homo sapiens (subdone 2_a6 from BAC H75) DNA sequence, complete sequence), D28485 (Human MSMB gene for beta-microseminoprotein (MSP), promoter region and exonl), and Z98052 (Human DNA sequence *** SEQUENCING IN PROGRESS *** from clone 505B13; HTGS phase 1). Based upon sequence similarity, Cb1101 proteins and each similar protein or peptide may share at least some activity.

Clone “ch4_{—}11”

A polynucleotide of the present invention has been identified as clone “ch4_{—}11”. ch4_{—}11 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ch4_{—}11 is a full-length clone, including the entire coding sequence of a secreted protein (also referred to herein as “ch4_{—}11 protein”).

The nucleotide sequence of ch4_{—}11 as presently determined is reported in SEQ ID NO:151, and includes a poly(A) tail. What applicants presently believe to be the proper reading frame and the predicted amino acid sequence of the ch4_{—}11 protein corresponding to the foregoing nucleotide sequence is reported in SEQ ID NO:152. Amino acids 21 to 33 of SEQ ID NO:152 are a predicted leader/signal sequence, with the predicted mature amino acid sequence beginning at amino acid 34. Due to the hydrophobic nature of the predicted leader/signal sequence, it is likely to act as a transmembrane domain should the predicted leader/signal sequence not be separated from the remainder of the ch4 11 protein.

The EcoRI/NotI restriction fragment obtainable from the deposit containing clone ch4_{—}11 should be approximately 1600 bp.

The nucleotide sequence disclosed herein for ch4_{—}11 was searched against the GenBank and GeneSeq nucleotide sequence databases using BLASTN/BLASTX and FASTA search protocols. ch4_{—}1I demonstrated at least some similarity with sequences identified as AA318160 (EST20431 Retina II Homo sapiens cDNA 5′ end), R94133 (yt74g06.r1 Soares fetal liver spleen 1NFLS Homo sapiens cDNA clone 276275 5′), and W27798 (37hl Human retina cDNA randomly primed sublibrary Homo sapiens). The predicted amino acid sequence disclosed herein for ch4_{—}11 was searched against the GenPept and GeneSeq amino acid sequence databases using the BLASTX search protocol. The predicted ch4_{—}1 protein demonstrated at least some similarity to sequences identified as L28819 (involucrin [Mus musculus]). The ch4_{—}1l protein is the human homologue of the mouse K483_{—}1 protein (see GenBank I80067 and I80068, GeneSeq V09119, V09120, and W42028, and U.S. Pat. No. 5,708,157). Based upon sequence similarity, ch4_{—}11 proteins and each similar protein or peptide may share at least some activity. The TopPredII computer program predicts three potential transmembrane domains within the ch4_{—}11 protein sequence centered around amino acids 28, 189, and 280 of SEQ ID NO:152, respectively.

Clone “cn621_{—}8”

A polynucleotide of the present invention has been identified as clone “cn621_{—}8”. cn621_{—}8 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cn621_{—}8 is a full-length clone, including the entire coding sequence of a secreted protein (also referred to herein as “cn621_{—}8 protein”). The nucleotide sequence of cn621_{—}8 as presently determined is reported in SEQ ID NO:153, and includes a poly(A) tail. What applicants presently believe to be the proper reading frame and the predicted amino acid sequence of the cn621 8 protein corresponding to the foregoing nucleotide sequence is reported in SEQ ID NO:154.

The EcoRI/NotI restriction fragment obtainable from the deposit containing clone cn621_{—}8 should be approximately 3500 bp.

The nucleotide sequence disclosed herein for cn621_{—}8 was searched against the GenBank and GeneSeq nucleotide sequence databases using BLASTN/BLASTX and FASTA search protocols. cn621_{—}8 demonstrated at least some similarity with sequences identified as WI 8181 (JMAGE:20099 Soares infant brain 1NIB Homo sapiens cDNA clone 20099), W60570 (zd26g04.r1 Soares fetal heart NbHH19W Homo sapiens cDNA clone 341814 5′), W60661 (zd26g04.s1 Soares fetal heart NbHH19W Homo sapiens cDNA clone), and Z84474 (Human DNA sequence from PAC 11lM5 on chromosome 6. Contains BBC1, RFP finger protein, EST, STS, tRNAs and polymorphic repeat). The predicted arnino acid sequence disclosed herein for cn621_{—}8 was searched against the GenPept and GeneSeq amino acid sequence databases using the BLASTX search protocol. The predicted cn621_{—}8 protein demonstrated at least some similarity to sequences identified as L35279 (BMP-1 [Homo sapiens]), U91963 (tolloid-like (TLL) [Homo sapiens]), and X64414 (low density lipoprotein receptor [Mus musculus]). Based upon sequence similarity, cn621_{—}8 proteins and each similar protein or peptide may share at least some activity. The TopPredII computer program predicts a potential transmembrane domain within the cn621_{—}8 protein sequence centered around amino acid 220 of SEQ ID NO:154.

Clone “g621_{—}1”

A polynucleotide of the present invention has been identified as clone “gy621_{—}1”. gy621_{—}1 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. gy621_{—}1 is a full-length clone, including the entire coding sequence of a secreted protein (also referred to herein as “gy621_{—}1 protein”).

The nucleotide sequence of gy621_{—}1 as presently determined is reported in SEQ ID NO:155, and includes a poly(A) tail. What applicants presently believe to be the proper reading frame and the predicted amino acid sequence of the gy621_{—}1 protein corresponding to the foregoing nucleotide sequence is reported in SEQ ID NO:156. Amino acids 11 to 23 of SEQ ID NO:156 are a predicted leader/signal sequence, with the predicted mature amino acid sequence beginning at amino acid 24. Due to the hydrophobic nature of the predicted leader/signal sequence, it is likely to act as a transmembrane domain should the predicted leader/signal sequence not be separated from the remainder of the gy621 1 protein.

The EcoRI/NotI restriction fragment obtainable from the deposit containing clone gy621_{—}1 should be approximately 3800 bp.

The nucleotide sequence disclosed herein for gy621_{—}1 was searched against the GenBank and GeneSeq nucleotide sequence databases using BLASTN/BLASTX and FASTA search protocols. gy621_{—}1 demonstrated at least some similarity with sequences identified as AA166536 (ms63h05.r1 Stratagene mouse embryonic carcinoma (#937317) Mus musculus cDNA clone 616281 5′), AA416723 (zuO8a04.s1 Soares testis NHT Homo sapiens cDNA clone 731214 3′), and AA463756 (aa07a05.r1 Soares NhHMPu S1 Homo sapiens cDNA clone 812528 5′). Based upon sequence similarity, gy621_{—}1 proteins and each similar protein or peptide may share at least some activity. The TopPredII computer program predicts at least one additional potential transmembrane domains within the gy621_{—}1 protein sequence of SEQ ID NO:156. The nucleotide sequence of gy621_{—}1 indicates that it may contain one or more ACI or AC2 repetitive elements.

Clone “hb1041_{—}2”

A polynucleotide of thepresentinventionhas been identified as clone “hb1041_{—}2”. hb1041_{—}2 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. hb1041_{—}2 is a full-length clone, including the entire coding sequence of a secreted protein (also referred to herein as “hb1041_{—}2 protein”).

The nucleotide sequence of hb1041_{—}2 as presently determined is reported in SEQ ID NO:157, and includes a poly(A) tail. What applicants presently believe to be the proper reading frame and the predicted amino acid sequence of the hb1041_{—}2 protein corresponding to the foregoing nucleotide sequence is reported in SEQ ID NO:158. Amino acids 55 to 67 of SEQ ID NO:158 are a predicted leader/signal sequence, with the predicted mature amino acid sequence beginning at amino acid 68. Due to the hydrophobic nature of the predicted leader/signal sequence, it may act as a transmembrane domain should the predicted leader/signal sequence not be separated from the remainder of the hb1041_{—}2 protein.

The EcoRI/NotI restriction fragment obtainable from the deposit containing clone hb1041_{—}2 should be approximately 2450 bp.

The nucleotide sequence disclosed herein for hb1040_{—}2 was searched against the GenBank and GeneSeq nucleotide sequence databases using BLASTN/BLASTX and FASTA search protocols. hb1041_{—}2 demonstrated at least some similarity with sequences identified as AA050445 (mj21c12.r1 Soares mouse embryo NbME13.5 14.5 Mus musculus cDNA clone 476758 5′), AA087161 (mo11b05.r1 Life Tech mouse embryo 10 5dpc 10665016 Mus musculus cDNA clone 553233 5′), and W84558 (zd89h10.s1 Soares fetal heart NbHH19W Homo sapiens cDNA clone 356707 3′). The predicted amino acid sequence disclosed herein for hb1041_{—}2 was searched against the GenPept and GeneSeq amino acid sequence databases using the BLASTX search protocol. The predicted hb1041_{—}2 protein demonstrated at least some similarity to sequences identified as AB000459 (unnamed protein product [Homo sapiens]). Based upon sequence similarity, hb1041_{—}2 proteins and each similar protein or peptide may share at least some activity.

Clone “mh703_{—}1”

A polynucleotide of the present invention has been identified as clone “mh703_{—}1”. mh703_{—}1 was isolated from a human adult brain (thalamus) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. mnh703_{—}1 is a full-length clone, including the entire coding sequence of a secreted protein (also referred to herein as “mh703_{—}1 protein”).

The nucleotide sequence of mh703_{—}1 as presently determined is reported in SEQ ID NO:159, and includes a poly(A) tail. What applicants presently believe to be the —proper reading frame and the predicted amino acid sequence of the mh703 1 protein corresponding to the foregoing nucleotide sequence is reported in SEQ ID NO:160.

The EcoRI/NotI restriction fragment obtainable from the deposit containing clone mnh703_{—}1 should be approximately 1700 bp.

The nucleotide sequence disclosed herein for mh703_{—}1 was searched against the GenBank and GeneSeq nucleotide sequence databases using BLASTN/BLASTX and FASTA search protocols. mh703_{—}1 demonstrated at least some similarity with sequences identified as AA173536 (zp04e07.r1 Stratagene ovarian cancer (#937219) Homo sapiens cDNA clone 595428 5′), AA173577 (zp04e07.s1 Stratagene ovarian cancer (#937219) Homo sapiens cDNA clone 595428 3′), AA278788 (zs79a09.r1 NCI_CGAP_GCB1 Homo sapiens cDNA clone IMAGE 703672 5′ similar to TR E189399 E189399 HYPOTHETICAL 51.4 KD PROTEIN), and T26646 (Human gene signature HUMGS08893). The predicted amino acid sequence disclosed herein for mh703_{—}1 was searched against the GenPept and GeneSeq amino acid sequence databases using the BLASTX search protocol. The predicted mh703_{—}1 protein demonstrated at least some similarity to sequences identified as R85881 (WD-40 domain-contg. YCW2 protein) and U80447 (similar to the beta transducin family [Caenorhabditis elegans]). mh703_{—}1 protein contains at least two beta-transducin family Trp-Asp repeat signature motifs, and also contains the WD-40 motif of G-proteins. Based upon sequence similarity, mh703_{—}1 proteins and each similar protein or peptide may share at least some activity. mh703_{—}1 protein was expressed in a COS cell expression system, and an expressed protein band of approximately 51 kDa was detected in conditioned medium using SDS polyacrylamide gel electrophoresis.

Clone “na461_{—}19”

A polynucleotide of the present invention has been identified as clone “na461_{—}19”. na461_{—}19 was isolated from a human adult brain (corpus callosum) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. na461_{—}19 is a full-length clone, including the entire coding sequence of a secreted protein (also referred to herein as “na461_{—}19 protein”).

The nucleotide sequence of na461_{—}19 as presently determined is reported in SEQ ID NO:161, and includes a poly(A) tail. What applicants presently believe to be the proper reading frame and the predicted amino acid sequence of the na461 19 protein corresponding to the foregoing nucleotide sequence is reported in SEQ ID NO:162.

Amino acids 63 to 75 of SEQ ID NO:162 are a predicted leader/signal sequence, with the predicted mature amino acid sequence beginning at amino acid 76. Due to the hydrophobic nature of the predicted leader/signal sequence, it is likely to act as a transmembrane domain should the predicted leader/signal sequence not be separated from the remainder of the na461_{—}19 protein.

The EcoRI/NotI restriction fragment obtainable from the deposit containing clone na461_{—}19 should be approximately 2300 bp.

The nucleotide sequence disclosed herein for na461_{—}19 was searched against the GenBank and GeneSeq nucleotide sequence databases using BLASTN/BLASTX and FASTA search protocols. na461_{—}19 demonstrated at least some similarity with sequences identified as AA032203 (zf01d04.s1 Soares fetal heart NbHH19W Homo sapiens cDNA clone 375655 3′), AA203707 (zx52c12.r1 Soares fetal liver spleen 1NFLS S1 Homo sapiens cDNA clone 446134 5′ similar to contains element MER2 repetitive element), AA262333 30 (zr70h11.s1 Soares NhHMPu S1 Homo sapiens cDNA clone 668805 3′), AA318276 (EST20340 Retina II Homo sapiens cDNA 5′ end), AA436588 (zv08e12.r1 Soares NhHMPu S1 Homo sapiens cDNA clone 753070 5′), and T21229 (Human gene signature HUMGS02545). Based upon sequence similarity, na461_{—}19 proteins and each similar protein or peptide may share at least some activity.

Clone “na492_{—}2”

A polynucleotide of the present invention has been identified as clone “na492_{—}2”. na492_{—}2 was isolated from a human adult brain (corpus callosum) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. na492_{—}2 is a full-length clone, including the entire coding sequence of a secreted protein (also referred to herein as “na492_{—}2 protein”).

The nucleotide sequence of na492_{—}2 as presently determined is reported in SEQ ID NO:163, and includes a poly(A) tail. What applicants presently believe to be the —proper reading frame and the predicted amino acid sequence of the na492 2 protein corresponding to the foregoing nucleotide sequence is reported in SEQ ID NO:164. Amino acids 321 to 333 of SEQ ID NO:164 are a predicted leader/signal sequence, with the predicted mature amino acid sequence beginning at amino acid 334. Due to the hydrophobic nature of the predicted leader/signal sequence, it is likely to act as a transmembrane domain should the predicted leader/signal sequence not be separated from the remainder of the na492_{—}2 protein.

The EcoRI/NotI restriction fragment obtainable from the deposit containing clone na492_{—}2 should be approximately 1800 bp.

The nucleotide sequence disclosed herein for na492_{—}2 was searched against the GenBank and GeneSeq nucleotide sequence databases using BLASTN/BLASTX and FASTA search protocols. na492_{—}2 demonstrated at least some similarity with sequences identified as AA514389 (nf57b05.s1 NCI_CGAP_Co3 Homo sapiens cDNA clone IMAGE:923985), H81154 (yu60f02.r1 Homo sapiens cDNA clone 230523 5′), and R89359 (yq05c05.s1 Homo sapiens cDNA clone 196040 3′). The predicted amino acid sequence disclosed herein for na492_{—}2 was searched against the GenPept and GeneSeq amino acid sequence databases using the BLASTX search protocol. The predicted na492_{—}2 protein demonstrated at least some similarity to sequences identified as AB004534 (pi015 [Schizosaccharomyces pombe]). Based upon sequence similarity, na492_{—}2 proteins and each similar protein or peptide may share at least some activity. The TopPredIl computer program predicts two potential transmembrane domains within the na492_{—}2 protein sequence, one centered around amino acid 350 and another around amino acid 370 of SEQ ID NO:164.

Clone “na669_{—}10”

A polynucleotide of the present invention has been identified as clone “na669_{—}10”. na669_{—}10 was isolated from a human adult brain (corpus callosum) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. na669_{—}10 is a full-length clone, including the entire coding sequence of a secreted protein (also referred to herein as “na669_{—}10 protein”).

The nucleotide sequence of na669_{—}10 as presently determined is reported in SEQ ID NO:165, and includes a poly(A) tail. What applicants presently believe to be the proper reading frame and the predicted amino acid sequence of the na669 10 protein corresponding to the foregoing nucleotide sequence is reported in SEQ ID NO:166. Amino acids 40 to 52 of SEQ ID NO:166 are a predicted leader/signal sequence, with the predicted mature amino acid sequence beginning at amino acid 53. Due to the hydrophobic nature of the predicted leader/signal sequence, it is likely to act as a transmembrane domain should the predicted leader/signal sequence not be separated from the remainder of the na669_{—}10 protein.

The EcoRI/NotI restriction fragment obtainable from the deposit containing clone na669_{—}10 should be approximately 3300 bp.

The nucleotide sequence disclosed herein for na669_{—}10 was searched against the GenBank and GeneSeq nucleotide sequence databases using BLASTN/BLASTX and FASTA search protocols. na669_{—}10 demonstrated at least some similarity with sequences identified as AA035207 (zk27h11.s1 Soares pregnant uterus NbHPU Homo sapiens cDNA clone 471813 3′), AA429797 (zw57d10.r1 Soares total fetus Nb2HF8 9w Homo sapiens cDNA clone 774163 5′), AA512946 (nh91d01.s1 NCI_CGAP_Br1.1 Homo sapiens cDNA clone JMAGE:965857), C20746 (HUMGS0004776, Human Gene Signature), and N33343 (yy08d08.s1 Homo sapiens cDNA clone 270639 3′). Based upon sequence similarity, na669_{—}10 proteins and each similar protein or peptide may share at least some activity. The TopPredII computer program predicts two potential transmembrane domains within the na669 10 protein sequence, one centered around amino acid 11 and another around amino acid 46 of SEQ ID NO:166.

Clone “co821_{—}31”

A polynucleotide of the present invention has been identified as clone “co821_{—}31”. co821_{—}31 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. co821_{—}31 is a full-length clone, including the entire coding sequence of a secreted protein (also referred to herein as “co821_{—}31 protein”).

The nucleotide sequence of co821_{—}31 as presently determined is reported in SEQ ID NO:167, and includes a poly(A) tail. What applicants presently believe to be the proper reading frame and the predicted amino acid sequence of the co821 31 protein corresponding to the foregoing nucleotide sequence is reported in SEQ ID NO:168. Amino acids 87 to 99 of SEQ ID NO:168 are a predicted leader/signal sequence, with the predicted mature amino acid sequence beginning at amino acid 100. Due to the hydrophobic nature of the predicted leader/signal sequence, it is likely to act as a transmembrane domain should the predicted leader/signal sequence not be separated from the remainder of the co821_{—}31 protein.

The EcoRI/NotI restriction fragment obtainable from the deposit containing clone co821_{—}31 should be approximately 2400 bp.

The nucleotide sequence disclosed herein for co821_{—}31 was searched against the GenBank and GeneSeq nucleotide sequence databases using BLASTN/BLASTX and FASTA search protocols. co821_{—}31 demonstrated at least some similarity with sequences identified as AA488906 (aa55a02.r1 NCI_CGAP_GCB1 Homo sapiens cDNA clone IMAGE:824810 5′ similar to TR:G607003 G607003 BETA TRANSDUCIN-LIKE PROTEIN), L26690 (Mus musculus expressed sequence tag EST F101), N30002 (yx82e02.s1 Homo sapiens cDNA clone 268250 3′), R82926 (EST23j22 Clontech adult human fat cell library HL1108A Homo sapiens cDNA clone 23j22), T20673 (Human gene signature HUMGS01889), and W44749 (zb98b11.s1 Soares parathyroid tumor NbHPA Homo sapiens cDNA clone 320829 3′). The predicted amino acid sequence disclosed herein for co821_{—}31 was searched against the GenPept and GeneSeq amino acid sequence databases using the BLASTX search protocol. The predicted co821_{—}31 protein demonstrated at least some similarity to sequences identified as U51030 (Ydr267cp [Saccharomyces cerevisiael). The predicted co821_{—}31 protein also demonstrated at least some similarity to U92792 (general transcriptional repressor Tup1 [Schizosaccharomyces pombe]), L28125 (beta transducin-like protein (het-el) [Podospora anserina]), and other proteins containing WD-40 motifs. Based upon sequence similarity, co821_{—}31 proteins and each similar protein or peptide may share at least some activity.

Clone “dk329_{—}1”

A polynucleotide of the present invention has been identified as clone “dk329_{—}1”. dk329_{—}1 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. dk329_{—}1 is a full-length clone, including the entire coding sequence of a secreted protein (also referred to herein as “dk329_{—}1 protein”).

The nucleotide sequence of dk329_{—}1 as presently determined is reported in SEQ ID NO:169, and includes a poly(A) tail. What applicants presently believe to be the proper reading frame and the predicted amino acid sequence of the dk329_{—}1 protein corresponding to the foregoing nucleotide sequence is reported in SEQ ID NO:170. Amino acids 71 to 83 of SEQ ID NO:170 are a predicted leader/signal sequence, with the predicted mature amino acid sequence beginning at arnino acid 84. Due to the hydrophobic nature of the predicted leader/signal sequence, it is likely to act as a transmembrane domain should the predicted leader/signal sequence not be separated from the remainder of the dk329_{—}1 protein.

The EcoRI/NotI restriction fragment obtainable from the deposit containing clone dk329_{—}1 should be approximately 1300 bp.

The nucleotide sequence disclosed herein for dk329_{—}1 was searched against the GenBank and GeneSeq nucleotide sequence databases using BLASTN/BLASTX and FASTA search protocols. dk329_{—}1 demonstrated at least some similarity with sequences identified as AA147429 (zo39g07.r1 Stratagene endothelial cell 937223 Homo sapiens cDNA clone 589308 5′ similar to WP T14G10.6 CE06452 LEUCOCYTE SURFACE ANTIGEN CD53 LINE), AA190572 (zp42h08.r1 Strata gene muscle 937209 Homo sapiens cDNA clone 612159 5′ similar to WP T14G10.6 CE06452 LEUCOCYTE SURFACE ANTIGEN CD53 LINE), AA234042 (zr51a05.s1 SoaresNhHMPu S1 Homo sapiens cDNA clone 666896 3′ similar to WP:T14G10.6 CE06452 LEUCOCYTE SURFACE ANTIGEN CD53 LINE), AA236262 (zr51a05.r1 Soares NhHMPu S1 Homo sapiens cDNA clone 666896 5′ similar to WP:T14G10.6 CE06452 LEUCOCYTE SURFACE ANTIGEN CD53 LINE), N72328 (yv31f12.r1 Homo sapiens cDNA clone 244367 5′ similar to SW A15_HUMANP41732 CELL SURFACE GLYCOPROTEINA15), and W50192 (mb08d07.r1 Life Tech mouse brain Mus musculus cDNA clone 319597 5′ similar to SW:CD53_HUMAN P19397 LEUKOCYTE SURFACE ANTIGEN CD53). The predicted amino acid sequence disclosed herein for dk329_{—}1 was searched against the GenPept and GeneSeq amino acid sequence databases using the BLASTX search protocol. The predicted dk329_{—}1 protein demonstrated at least some similarity to sequences identified as Z68880 (T14G10.6 [Caenorhabditis elegans]) and a variety of membrane proteins involved in immune function. Based upon sequence similarity, dk329_{—}1 proteins and each similar protein or peptide may share at least some activity. The TopPredII computer program predicts three potential transmembrane domains within the dk329_{—}1 protein sequence, centered around amino acids 31, 71, and 103 of SEQ ID NO:170, respectively.

dk329_{—}1 protein was expressed in a COS cell expression system, and an expressed protein band of approximately 18 kDa was detected in membrane fractions using SDS polyacrylamide gel electrophoresis.

Clone “fx317_{—}11”

A polynucleotide of the present invention has been identified as clone “fx317_{—}11”. fx317_{—}11 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fx317_{—}11 is a full-length clone, including the entire coding sequence of a secreted protein (also referred to herein as “fx317_{—}11 protein”).

The nucleotide sequence of fx317_{—}11 as presently determined is reported in SEQ ID NO:171, and includes a poly(A) tail. What applicants presently believe to be the proper reading frame and the predicted amino acid sequence of the fx317_{—}11 protein corresponding to the foregoing nucleotide sequence is reported in SEQ ID NO:172. Amino acids 229 to 241 of SEQ ID NO:172 are a predicted leader/signal sequence, with the predicted mature amino acid sequence beginning at amino acid 242. Due to the hydrophobic nature of the predicted leader/signal sequence, it is likely to act as a transmembrane domain should the predicted leader/signal sequence not be separated from the remainder of the fx317_{—}11 protein.

The EcoRI/NotI restriction fragment obtainable from the deposit containing clone fx317_{—}11 should be approximately 1900 bp.

The nucleotide sequence disclosed herein for fx317_{—}11 was searched against the GenBank and GeneSeq nucleotide sequence databases using BLASTN/BLASTX and FASTA search protocols. fx317_{—}11 demonstrated at least some similarity with sequences identified as AA505600 (nh93h11.s1 NCI_CGAP_Br2 Homo sapiens cDNA clone IMAGE:966117), N47450 (yy89c09.r1 Homo sapiens cDNA clone 280720 5′ similar to contains element PTR5 repetitive element), T64549 (Human activated platelet protein-2 APP-2 cDNA), and W52611 (zc49e02.r1 Soares senescent fibroblasts NbHSF Homo sapiens cDNA clone 325658 5′). The predicted amino acid sequence disclosed herein for fx317_{—}11 was searched against the GenPept and GeneSeq amino acid sequence databases using the BLASTX search protocol. The predicted fx317_{—}11 protein demonstrated at least some similarity to sequences identified as W15413 (Human activated platelet protein-2 APP-2) and W15414 (Human activated platelet protein-2 APP-2 alternatively spliced variant). APP-2 protein is expressed on activated human platelets. Based upon sequence similarity, fx317_{—}11 proteins and each sirnilar protein or peptide may share at least some activity.

Clone “lp547_{—}4”

A polynucleotide of the present invention has been identified as clone “lp547_{—}4”. lp547_{—}4 was isolated from a human adult blood (peripheral blood mononuclear cells treated in vivo with granulocyte-colony stimulating factor) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. lp547_{—}4 is a full-length clone, including the entire coding sequence of a secreted protein (also referred to herein as “lp547_{—}4 protein”).

The nucleotide sequence of lp547_{—}4 as presently determined is reported in SEQ ID NO:173, and includes a poly(A) tail. What applicants presently believe to be the proper reading frame and the predicted amino acid sequence of the lp547_{—}4 protein corresponding to the foregoing nucleotide sequence is reported in SEQ ID NO:174.

The EcoRI/NotI restriction fragment obtainable from the deposit containing clone lp547_{—}4 should be approximately 1800 bp.

The nucleotide sequence disclosed herein for lp547_{—}4 was searched against the GenBank and GeneSeq nucleotide sequence databases using BLASTN/BLASTX and FASTA search protocols. lp547_{—}4 demonstrated at least some similarity with sequences identified as AA442560 (zv75g07.r1 Soares total fetus Nb2HF8 9w Homo sapiens cDNA clone 759516 5′ similar to TR:G436941 G436941 PHORBOLIN I). The predicted amino acid sequence disclosed herein for lp547_{—}4 was searched against the GenPept and GeneSeq amino acid sequence databases using the BLASTX search protocol. The predicted lp547_{—}4 protein demonstrated at least some similarity to sequences identified as R58704 (Apo-B RNA editing protein), U03891 (phorbolin I [Homo sapiens]), and U21951 (apolipoprotein B mRNA-editing component 1 [Mus musculus]). U03891 protein (phorbolin I) is upregulated in psoriatic keratinocytes. The predicted lp547_{—}4 protein also contains a cytidine and deoxycytidylate deaminases zinc-binding region signature. Based upon sequence similarity, lp547_{—}4 proteins and each similar protein or peptide may share at least some activity. The TopPredII computer program predicts a potential transmembrane domain within the lp547_{—}4 protein sequence, centered around amino acid 290 of SEQ ID NO:174; amino acids 278 to 290 are also a possible leader/signal sequence, with the predicted mature amino acid sequence beginning at amino acid 291.

lp547_{—}4 protein was expressed in a COS cell expression system, and an expressed protein band of approximately 41 kDa was detected in conditioned medium and membrane fractions using SDS polyacrylamide gel electrophoresis.

Clone “lv310_{—}7”

A polynucleotide of the present invention has been identified as clone “lv310_{—}7”. Clones were first isolated from a human adult thyroid cDNA library using methods which are selective for cDNAs encoding secreted proteins (see U.S. Pat. No. 5,536,637), or were identified as encoding a secreted or transmembrane protein on the basis of computer analysis of the amino acid sequence of the encoded protein. Probes derived from these cDNAs were then used to isolate lv310_{—}7 from a human adult brain cDNA library. lv310_{—}7 is a full-length clone, including the entire coding sequence of a secreted protein (also referred to herein as “lv310_{—}7 protein”).

The nucleotide sequence of lv3107 as presently determined is reported in SEQ ID NO:175, and includes a poly(A) tail. What applicants presently believe to be the proper reading frame and the predicted amino acid sequence of the lv31l_{—}7 protein corresponding to the foregoing nucleotide sequence is reported in SEQ ID NO:176. Amino acids 269 to 281 of SEQ ID NO:176 are a predicted leader/signal sequence, with the predicted mature amino acid sequence beginning at amino acid 282. Due to the hydrophobic nature of the predicted leader/signal sequence, it is likely to act as a transmembrane domain should the predicted leader/signal sequence not be separated from the remainder of the lv310l7 protein.

Another possible lv310_{—}7 reading frame and predicted amino acid sequence, encoded by base pairs 1619 to 2188 of SEQ ID NO:175, is reported in SEQ ID NO:283.

The EcoRI/NotI restriction fragment obtainable from the deposit containing clone lv310_{—}7 should be approximately 3650 bp.

The nucleotide sequence disclosed herein for lv310_{—}7 was searched against the GenBank and GeneSeq nucleotide sequence databases using BLASTN/BLASTX and FASTA search protocols. lv310_{—}7 demonstrated at least some similarity with sequences identified as N37001 (yy40a01.s1 Homo sapiens cDNA clone 273672 3′), R56228 (yg90d01.s1 Homo sapiens cDNA clone 40958 3′), and R56310 (yg90d01.r1 Homo sapiens cDNA clone 40958 5′). The predicted amino acid sequence disclosed herein for lv310_{—}7 was searched against the GenPept and GeneSeq amino acid sequence databases using the BLASTX search protocol. The predicted lv310_{—}7 protein demonstrated at least some similarity to sequences identified as U24223 (alpha-CPl [Homo sapiens]). Based upon sequence similarity, lv310_{—}7 proteins and each similar protein or peptide may share at least some activity. The TopPredII computer program predicts 10 potential transmembrane domains within the lv310_{—}7 protein sequence, centered around amino acids 100, 130, 160, 210, 280, 490, 520, 600, 690, and 750 of SEQ ID NO:176, respectively.

Clone “nq34_{—}12”

A polynucleotide of the present invention has been identified as clone “nq34_{—}12”. nq34_{—}12 was isolated from a human adult blood (erythroleukemia TF-1) cDNA library using methods which are selective for cDNAs encoding secreted proteins (see U.S. Pat. No.5,536,637), or was identified as encoding a secreted or transmembrane protein on the basis of computer analysis of the amino acid sequence of the encoded protein. nq34_{—}12 is a full-length clone, including the entire coding sequence of a secreted protein (also referred to herein as “nq34_{—}12 protein”).

The nucleotide sequence of nq34_{—}12 as presently determined is reported in SEQ ID NO:177, and includes a poly(A) tail. What applicants presently believe to be the proper reading frame and the predicted amino acid sequence of the nq34_{—}12 protein corresponding to the foregoing nucleotide sequence is reported in SEQ ID NO:178. Amino acids 287 to 299 of SEQ ID NO:178 are a predicted leader/signal sequence, with —the predicted mature amino acid sequence beginning at amino acid 300. Due to the hydrophobic nature of the predicted leader/signal sequence, it is likely to act as a transmembrane domain should the predicted leader/signal sequence not be separated from the remainder of the nq34_{—}12 protein.

The EcoRI/NotI restriction fragment obtainable from the deposit containing clone nq34 12 should be approximately 1700 bp.

The nucleotide sequence disclosed herein for nq34_{—}12 was searched against the GenBank and GeneSeq nucleotide sequence databases using BLASTN/BLASTX and FASTA search protocols. nq34_{—}12 demonstrated at least some similarity with sequences identified as AA126375 (zl86c06.r1 Stratagene colon (#937204) Homo sapiens cDNA clone 511498 5′), AA446675 (zw84a08.r1 Soares total fetus Nb2HF8 9w Homo sapiens cDNA clone 783638 5′), AA448974 (zx07d05.r1 Soares total fetus Nb2HF8 9w Homo sapiens cDNA clone 785769 5′ similar to SW YNDO_YEAST P40344 HYPOTHETICAL 35.9 KD PROTEIN IN RPC34-CSE2 INTERGENIC REGION), R57902 (F6699 Fetal heart Homo sapiens cDNA clone F6699 5′ end), and X07453 (Plasmodium falciparum 11_{—}1 gene part 1). The predicted amino acid sequence disclosed herein for nq34_{—}12 was searched against the GenPept and GeneSeq amino acid sequence databases using the BLASTX search protocol. The predicted nq34_{—}12 protein demonstrated at least some similarity to sequences identified as X77395 (N2040 gene product [Saccharomyces cerevisiae]). Based upon sequence similarity, nq34_{—}12 proteins and each similar protein or peptide may share at least some activity. nq34_{—}12 protein was expressed in a COS cell expression system, and an expressed protein band of approximately 34 kDa was detected in membrane fractions using SDS polyacrylamide gel electrophoresis.

Clone “pj154_{—}1”

A polynucleotide of the present invention has been identified as clone “pj154_{—}1”. pj154_{—}1 was isolated from a human fetal carcinoma (NTD2 cells treated with retinoic acid for 23 days) cDNA library using methods which are selective for cDNAs encoding secreted proteins (see U.S. Pat. No.5,536,637), or was identified as encoding a secreted or transmembrane protein on the basis of computer analysis of the amino acid sequence of the encoded protein. pj154_{—}1 is a full-length clone, including the entire coding sequence of a secreted protein (also referred to herein as “pj154 1 protein”).

The nucleotide sequence of pj154_{—}1 as presently determined is reported in SEQ ID NO:179, and includes a poly(A) tail. What applicants presently believe to be the proper reading frame and the predicted amino acid sequence of the pj154_{—}1 protein corresponding to the foregoing nucleotide sequence is reported in SEQ ID NO:180. Amino acids 13 to 25 of SEQ ID NO:180 are a predicted leader/signal sequence, with the predicted mature amino acid sequence beginning at amnino acid 26. Due to the hydrophobic nature of the predicted leader/signal sequence, it is likely to act as a transmembrane domain should the predicted leader/signal sequence not be separated from the remainder of the pj154_{—}1 protein.

The EcoRI/NotI restriction fragment obtainable from the deposit containing clone pj154_{—}1 should be approximately 2300 bp.

The nucleotide sequence disclosed herein for pj154_{—}1 was searched against the GenBank and GeneSeq nucleotide sequence databases using BLASTN/BLASTX and FASTA search protocols. pj154_{—}1 demonstrated at least some similarity with sequences identified as AA223153 (zr07g12.r1 Stratagene NT2 neuronal precursor 937230 Homo sapiens cDNA clone 650854 5′), AA223170 (zr07g12.s1 Stratagene NT2 neuronal precursor 937230 Homo sapiens cDNA clone 650854 3′ similar to contains Alu repetitive element), H16627 (ym26d04.r1 Homo sapiens cDNA clone 49469 5′), and Z44660 (H. sapiens partial cDNA sequence; clone c-26d11). Based upon sequence similarity, pj154_{—}1 proteins and each similar protein or peptide may share at least some activity. The nucleotide sequence of pj154_{—}1 indicates that it may contain an Alu repetitive element.

Clone “pk147_{—}1”

A polynucleotide of the present invention has been identified as clone “pk147_{—}1”. pk147_{—}1 was isolated from a human fetal kidney (293 cell line) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. pk147_{—}1 is a full-length clone, including the entire coding sequence of a secreted protein (also referred to herein as “pk147_{—}1 protein”).

The nucleotide sequence of pk147_{—}1 as presently determined is reported in SEQ ID NO:181, and includes a poly(A) tail. What applicants presently believe to be the proper reading frame and the predicted amino acid sequence of the pk147_{—}1 protein corresponding to the foregoing nucleotide sequence is reported in SEQ ID NO:182. Amino acids 16 to 28 of SEQ ID NO:182 are a predicted leader/signal sequence, with the predicted mature amino acid sequence beginning at amino acid 29. Due to the hydrophobic nature of the predicted leader/signal sequence, it is likely to act as a transmembrane domain should the predicted leader/signal sequence not be separated from the remainder of the pk147_{—}1 protein.

The EcoRI/NotI restriction fragment obtainable from the deposit containing clone pk147_{—}1 should be approximately 1600 bp.

The nucleotide sequence disclosed herein for pk147_{—}1 was searched against the GenBank and GeneSeq nucleotide sequence databases using BLASTN/BLASTX and FASTA search protocols. pk147_{—}1 demonstrated at least some similarity with sequences identified as AA126920 (z123h01.s1 Soares pregnant uterus NbHPU Homo sapiens cDNA clone 502801 3′), AA406448 (zv12f07.r1 Soares NhHMPu S1 Homo sapiens cDNA clone 753445 5′), and R51886 (yg78c03.s1 Homo sapiens cDNA clone 39574 3′). Based upon sequence similarity, pk147_{—}1 proteins and each similar protein or peptide may share at least some activity. The TopPredII computer program predicts an additional potential transmembrane domain within the pk147_{—}1 protein sequence centered around amino acid 37 of SEQ ID NC:182.

Clone “pt127_{—}1”

A polynucleotide of the present invention has been identified as clone “pt127_{—}1”. pt127_{—}1 was isolated from a human adultblood (lymphoblastic leukemia MOLT-4) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. pt127_{—}1 is a full-length clone, including the entire coding sequence of a secreted protein (also referred to herein as “pt127_{—}1 protein”).

The nudeotide sequence of pt127_{—}1 as presently determined is reported in SEQ ID NO:183, and includes a poly(A) tail. What applicants presently believe to be the proper reading frame and the predicted amino acid sequence of the pt127_{—}1 protein corresponding to the foregoing nucleotide sequence is reported in SEQ ID NO:184. Amnino acids 8 to 20 of SEQ ID NO:184 are a predicted leader/signal sequence, with the predicted mature amino acid sequence beginning at amino acid 21. Due to the hydrophobic nature of the predicted leader/signal sequence, it is likely to act as a transmembrane domain should the predicted leader/signal sequence not be separated from the remainder of the pt127_{—}1 protein.

The EcoRI/NotI restriction fragment obtainable from the deposit containing clone pt127_{—}1 should be approximately 2600 bp.

The nucleotide sequence disclosed herein for pt127_{—}1 was searched against the GenBank and GeneSeq nucleotide sequence databases using BLASTN/BLASTX and FASTA search protocols. pt127_{—}1 demonstrated at least some similarity with sequences identified as AA081843 (zn19g10.r1 Stratagene neuroepitheliumNT2RAMI 937234 Homo sapiens cDNA clone 547938 5′) and R39258 (yc91h08.s1 Homo sapiens cDNA clone 23514 3′). Based upon sequence similarity, pt127_{—}1 proteins and each similar protein or peptide may share at least some activity. The TopPredII computer program predicts five additional potential transmembrane domains within the pt127_{—}1 protein sequence centered around amino acids 60, 100, 130, 190, and 240 of SEQ ID NO:184.

Clone “qo115_{—}13”

A polynucleotide of the present invention has been identified as clone “qo115_{—}13”. qo115_{—}13 was isolated from a human adult brain (corpus callosum) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. qo115_{—}13 is a full-length clone, including the entire coding sequence of a secreted protein (also referred to herein as “qo115_{—}13 protein”). The nucleotide sequence of qo115_{—}13 as presently determined is reported in SEQ ID NO:185, and includes a poly(A) tail. What applicants presently believe to be the proper reading frame and the predicted amino acid sequence of the qo115_{—}13 protein corresponding to the foregoing nucleotide sequence is reported in SEQ ID NO:186. Amino acids 29 to 41 of SEQ ID NO:186 are a predicted leader/signal sequence, with the predicted mature amino acid sequence beginning at amino acid 42. Due to the hydrophobic nature of the predicted leader/signal sequence, it is likely to act as a transmembrane domain should the predicted leader/signal sequence not be separated from the remainder of the qo115_{—}3 protein.

The EcoRI/NotI restriction fragment obtainable from the deposit containing clone qo115_{—}13 should be approximately 1200 bp.

The nucleotide sequence disclosed herein for qo115_{—}13 was searched against the GenBank and GeneSeq nucleotide sequence databases using BLASTN/BLASTX and FASTA search protocols. No significant hits were found in the database. The nucleotide sequence of qo115_{—}13 indicates that it may contain repetitive elements.

Deposit of Clones

Clones bd306_{—}7, fj283_{—}11, fk317_{—}3, k213_{—}2x, na316_{—}1, nf93_{—}20, np164_{—}1, pe204_{—}1, ya1_{—}1, and yb8_{—}1 were deposited on Nov. 26,1997 with the ATCC (American Type Culture Collection, 10801 University Boulevard, Manassas, Va. 20110-2209 U.S.A.) as an original deposit under the Budapest Treaty and were given the accession number 98599, fromwhich each clone comprising a particular polynucleotide is obtainable. Clone fj283_{—}6 was deposited on Nov. 17, 1998 with the ATCC (American Type Culture Collection, 10801 University Boulevard, Manassas, Va. 20110-2209 U.S.A.) as an original deposit under the Budapest Treaty and was given the accession number 98988.

Clones am856_{—}3, am996_{—}12, cc69_{—}1, cc162_{—}1, if87_{—}1, nn103_{—}4, np206_{—}8, nt746_{—}4, pe286_{—}1, and yb7_{—}1 were deposited on Dec. 4,1997 with the ATCC (American Type Culture Collection, 10801 University Boulevard, Manassas, Va. 20110-2209 U.S.A.) as an original deposit under the Budapest Treaty and were given the accession number 98600, from which each clone comprising a particular polynucleotide is obtainable.

Clones am728_{—}60, bf377_{—}1, cw354_{—}1, nml34_{—}4,yb11_{—}1, and yc2_{—}1 were deposited on Dec. 19, 1997 with the ATCC (American Type Culture Collection, 10801 University Boulevard, Manassas, Va. 20110-2209 U.S.A.) as an original deposit under the Budapest Treaty and were given the accession number 98621, from which each clone comprising a particular polynucleotide is obtainable.

Clones ff168_{—}12, ls9_{—}1, na1010_{—}1, nf87_{—}1, nh796_{—}1, nn229_{—}1, and np156_{—}1 were deposited on Dec. 31, 1997 with the ATCC (American Type Culture Collection, 10801 University Boulevard, Manassas, Va. 20110-2209 U.S.A.) as an original deposit under the Budapest Treaty and were given the accession number 98623, from which each clone comprising a particular polynucleotide is obtainable.

Clones bg570_{—}1,bi120_{—}2,bn594_{—}1, en554_{—}1, na474_{—}10, nn16_{—}10, np189_{—}9, ny226_{—}1, pe159_{—}1, and pj314_{—}8 were deposited on Jan. 7,1998 with the ATCC (American Type Culture Collection, 10801 University Boulevard, Manassas, Virginia 20110_{—}2209 U.S.A.) as an original deposit under the Budapest Treaty and were given the accession number 98629, from which each clone comprising a particular polynucleotide is obtainable.

Clones bp870 2, bx141l2, cw272_{—}7, nh328 5, nnn214 3, nn320 2, pp392 3, yal31, yb37_{—}1, and yb39_{—}1 were deposited on Jan. 8, 1998 with the American Type Culture Collection, 10801 University Boulevard, Manassas, Va. 20110_{—}2209 U.S.A.) as an original deposit under the Budapest Treaty and were given the accession number 98630, from which each clone comprising a particular polynucleotide is obtainable. Clone bp870_{—}1 was deposited on Apr. 7, 1998 with the ATCC (American Type Culture Collection, 10801 University Boulevard, Manassas, Virginia 20110-2209 U.S.A.) as an original deposit under the Budapest Treaty and was given the accession number 98724, from which deposit the bp870_{—}1 clone comprising a particular polynucleotide is obtainable.

Clones bd577_{—}1, bv280_{—}3, co315_{—}3, ij226_{—}6, nf443_{—}1, nt429_{—}1, pe503_{—}1, pe834_{—}6, ya10_{—}1l, and yb40_{—}1 were deposited on Jan. 13, 1998 with the ATCC (American Type Culture Collection, 10801 University Boulevard, Manassas, Va. 20110-2209 U.S.A.) as an original deposit under the Budapest Treaty and were given the accession number 98631, from which each clone comprising a particular polynucleotide is obtainable.

Clones cs756_{—}2, ew150_{—}1, gg894_{—}13, it217_{—}2, ml235_{—}2, mt24_{—}2, pe584_{—}2, pj323_{—}2, yb24_{—}1, and yb44_{—}1 were deposited on Jan. 22,1998 with the ATCC (American Type Culture Collection, 10801 University Boulevard, Manassas, Va. 20110-2209 U.S.A.) as an original deposit under the Budapest Treaty and were given the accession number 98636, from which each clone comprising a particular polynucleotide is obtainable.

Clones bn69_{—}15, cb110_{—}1, ch4_{—}11, cn621_{—}8, gy621_{—}1, hb104_{—}2, mh703_{—}1, na461_{—}19, na492_{—}2, and na669_{—}10 were deposited on Jan. 30, 1998 with the ATCC (American Type Culture Collection, 10801 University Boulevard, Manassas, Va. 20110-2209 U.S.A.) as an original deposit under the Budapest Treaty and were given the accession number 98647, from which each clone comprising a particular polynucleotide is obtainable.

Clones co821_{—}31, dk329_{—}1, fx317_{—}11, lp547_{—}4, lv310_{—}7, nq34_{—}12, pj154_{—}1, pk147_{—}1, pt127_{—}1, and qo115_{—}13 were deposited on Feb. 18, 1998 with the American Type Culture Collection (10801 University Boulevard, Manassas, Va. 20110-2209 U.S.A.) as an original deposit under the Budapest Treaty and were given the accession number ATCC 98663, from which each clone comprising a particular polynucleotide is obtainable.

All restrictions on the availability to the public of the deposited material will be irrevocably removed upon the granting of the patent, except for the requirements specified in 37 C.F.R. § 1.808(b), and the term of the deposit will comply with 37 C.F.R. § 1.806.

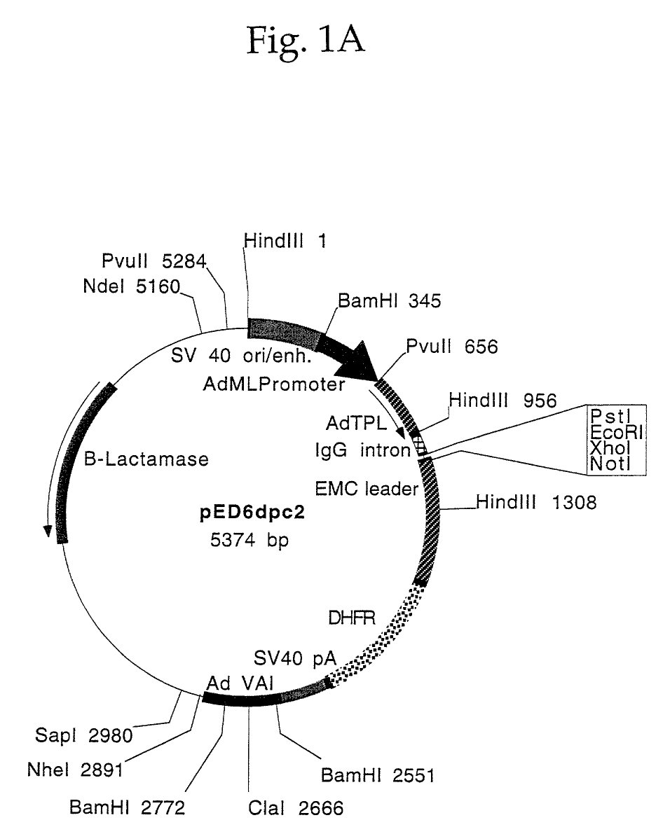

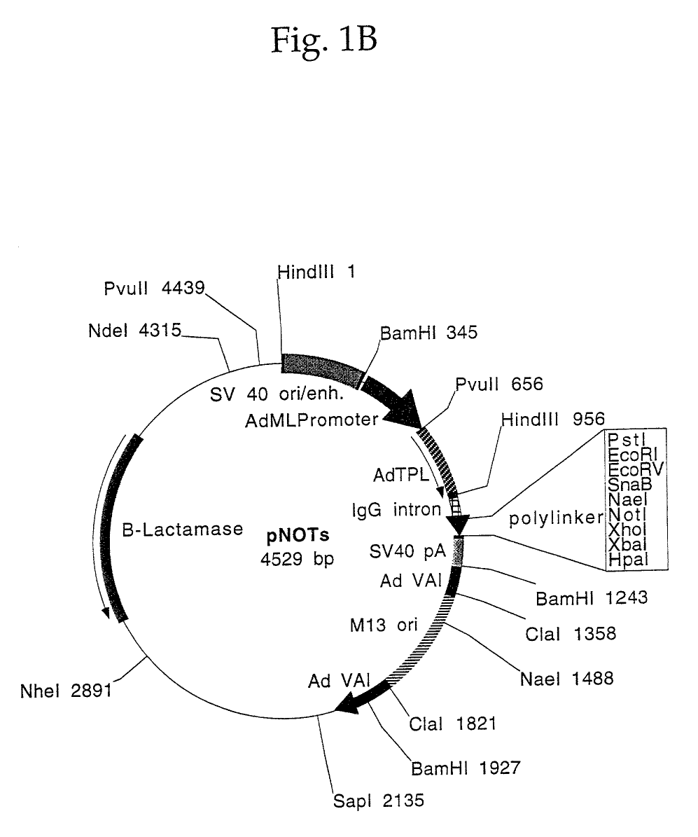

Each clone has been transfected into separate bacterial cells (E. coli) in these composite deposits. Each clone can be removed from the vector in which it was deposited by performing an EcoRI/NotI digestion (5′ site, EcoRI; 3′ site, NotI) to produce the appropriate fragment for such clone. Each clone was deposited in either the pED6 or pNOTs vector depicted in FIGS. 1A and 1B, respectively. The pED6dpc2 vector (“pED6”) was derived from pED6dpcl by insertion of a new polylinker to facilitate cDNA cloning (Kaufman et al., 1991, Nucleic Acids Res. 19: 4485_{—}4490); the pNOTs vector was derived from pMT2 (Kaufman et al., 1989, Mol. Cell. Biol. 9: 946-958) by deletion of the DHFR sequences, insertion of a new polylinker, and insertion of the M13 origin of replication in the Clal site. In some instances, the deposited clone can become “flipped” (i.e., in the reverse orientation) in the deposited isolate. In such instances, the cDNA insert can still be isolated by digestion with EcoRI and NotI. However, NotI will then produce the 5′ site and EcoR will produce the 3′ site for placement of the cDNA in proper orientation for expression in a suitable vector. The cDNA may also be expressed from the vectors in which they were deposited.

Bacterial cells containing a particular clone can be obtained from the composite deposit as follows:

An oligonucleotide probe or probes should be designed to the sequence that is known for that particular clone. This sequence can be derived from the sequences provided herein, or from a combination of those sequences. The sequence of an oligonucleotide probe that was used to isolate or to sequence each full-length clone is identified below, and should be most reliable in isolating the clone of interest.

| Clone | Probe Sequence |
|---|---|
| bd306_7 | SEQ ID NO: 187 |
| fj283_11 | SEQ ID NO: 188 |
| p283_6 | SEQ ID NO: 197 |
| fk317_3 | SEQ ID NO: 189 |
| k213_2x | SEQ ID NO: 190 |
| na316_1 | SEQ ID NO: 191 |
| nf93_20 | SEQ ID NO: 192 |
| np164_1 | SEQ ID NO: 193 |
| pe204_1 | SEQ ID NO: 194 |
| ya1_1 | SEQ ID NO: 195 |
| yb8_1 | SEQ ID NO: 196 |
| am856_3 | SEQ ID NO: 199 |
| am996_12 | SEQ ID NO: 200 |
| cc69_1 | SEQ ID NO: 201 |
| cc162_1 | SEQ ID NO: 202 |
| if87_1 | SEQ ID NO: 203 |
| nnlO3_4 | SEQ ID NO: 204 |
| np206_8 | SEQ ID NO: 205 |
| nt746_4 | SEQ ID NO: 206 |
| pe286_1 | SEQ ID NO: 207 |
| yb7_1 | SEQ ID NO: 208 |
| am728_60 | SEQ ID NO: 209 |
| cw354_1 | SEQ ID NO: 210 |
| nm134_4 | SEQ ID NO: 211 |
| yb11_1 | SEQ ID NO: 212 |
| yc2_1 | SEQ ID NO: 213 |
| ff168_12 | SEQ ID NO: 214 |
| 1s9_1 | SEQ ID NO: 215 |
| na1010_1 | SEQ ID NO: 216 |
| nf87_1 | SEQ ID NO: 217 |
| nh796_1 | SEQ ID NO: 218 |
| nn229_1 | SEQ ID NO: 219 |
| np156_1 | SEQ ID NO: 220 |
| bi120_2 | SEQ ID NO: 221 |
| na474_10 | SEQ ID NO: 222 |
| nnl6_10 | SEQ ID NO: 223 |
| np189_9 | SEQ ID NO: 224 |
| ny226_1 | SEQ ID NO: 225 |
| pe159_1 | SEQ ID NO: 226 |
| pj314_8 | SEQ ID NO: 227 |
| bp870_1 | SEQ ID NO: 228 |
| bx141_2 | SEQ ID NO: 229 |
| cw272_7 | SEQ ID NO: 230 |
| nh328_5 | SEQ ID NO: 231 |
| nm214_3 | SEQ ID NO: 232 |
| nn320_2 | SEQ ID NO: 233 |
| pp392_3 | SEQ ID NO: 234 |
| yb37_1 | SEQ ID NO: 235 |
| bd577_1 | SEQ ID NO: 236 |
| bv280_3 | SEQ ID NO: 237 |
| co315_3 | SEQ ID NO: 238 |
| ij226_6 | SEQ ID NO: 239 |
| nf443_1 | SEQ ID NO: 240 |
| nt429_1 | SEQ ID NO: 241 |
| pe503_1 | SEQ ID NO: 242 |
| pe834_6 | SEQ ID NO: 243 |
| yb40_1 | SEQ ID NO: 244 |
| cs756_2 | SEQ ID NO: 245 |
| ew150_1 | SEQ ID NO: 246 |
| gg894_13 | SEQ ID NO: 247 |
| it217_2 | SEQ ID NO: 248 |
| ml235_2 | SEQ ID NO: 249 |
| mt24_2 | SEQ ID NO: 250 |
| pe584_2 | SEQ ID NO: 251 |
| pj323_2 | SEQ ID NO: 252 |
| yb24_1 | SEQ ID NO: 253 |
| bn69_15 | SEQ ID NO: 254 |
| cb110_1 | SEQ ID NO: 255 |
| ch4_11 | SEQ ID NO: 256 |
| cn621_8 | SEQ ID NO: 257 |
| gy621_1 | SEQ ID NO: 258 |
| hb1041_2 | SEQ ID NO: 259 |
| mh703_1 | SEQ ID NO: 260 |
| na461_19 | SEQ ID NO: 261 |
| na492_2 | SEQ ID NO: 262 |
| na669_10 | SEQ ID NO: 263 |
| co821_31 | SEQ ID NO: 264 |
| dk329_1 | SEQ ID NO: 265 |
| fx317_11 | SEQ ID NO: 266 |
| lp547_4 | SEQ ID NO: 267 |
| lv310_7 | SEQ ID NO: 268 |
| nq342_12 | SEQ ID NO: 269 |
| pj154_1 | SEQ ID NO: 270 |
| pk147_1 | SEQ ID NO: 271 |
| pt127_1 | SEQ ID NO: 272 |
| qo115_13 | SEQ ID NO: 273 |

In the sequences listed above which include an N at position 2, that position is occupied in preferred probes/primers by a biotinylated phosphoaramidite residue rather than a nucleotide (such as, for example, that produced by use of biotin phosphoramidite (1-dimethoxytrityloxy-2-(N-biotinyl-4-aminobutyl)-propyl-3-O-(2-cyanoethyl)-(N,N-diisopropyl)-phosphoramadite) (Glen Research, cat. no. 10-1953)).

The design of the oligonucleotide probe should preferably follow these parameters:

(a) It should be designed to an area of the sequence which has the fewest ambiguous bases (“N's”), if any;

(b) It should be designed to have a Tₘ of approx. 80 C (assuming 2 for each A or T and 4 degrees for each G or C).

The oligonucleotide should preferably be labeled with γ-³²P ATP (specific activity 6000 Ci/mmole) and T4 polynucleotide kinase using commonly employed techniques for labeling oligonucleotides. Other labeling techniques can also be used. Unincorporated label should preferably be removed by gel filtration chromatography or other established methods. The amount of radioactivity incorporated into the probe should be quantitated by measurement in a scintillation counter. Preferably, specific activity of the resulting probe should be approximately 4e+6 dpm/pmole.

The bacterial culture containing the pool of full-length clones should preferably be thawed and 100 μl of the stock used to inoculate a sterile culture flask containing 25 ml of sterile L-broth containing ampicillin at 100 μg/ml. The culture should preferably be grown to saturation at 37° C., and the saturated culture should preferably be diluted in fresh L-broth. Aliquots of these dilutions should preferably be plated to determine the dilution and volume which will yield approximately 5000 distinct and well-separated colonies on solid bacteriological media containing L-broth containing ampicillin at 100 μg/ml and agar at 1.5% in a 150 mm petri dish when grown overnight at 37° C. Other known methods of obtaining distinct, well-separated colonies can also be employed.

Standard colony hybridization procedures should then be used to transfer the colonies to nitrocellulose filters and lyse, denature and bake them.

The filter is then preferably incubated at 65° C. for 1 hour with gentle agitation in 6×SSC (20×stock is 175.3 g NaCl/liter, 88.2 g Na citrate/liter, adjusted to pH 7.0 with NaOH) containing 0.5% SDS, 100 μg/ml of yeast RNA, and 10 mM EDTA (approximately mL per 150 mm filter). Preferably, the probe is then added to the hybridization mix at a concentration greater than or equal to le+6 dpm/mL. The filter is then preferably incubated at 65° C. with gentle agitation overnight. The filter is then preferably washed in 500 mL of 2)(SSC/0.5% SDS at room temperature without agitation, preferably followed by 500 mL of 2×SSC/0.1% SDS at room temperature with gentle shaking for 15 minutes. A third wash with 0.1×SSC/0.5% SDS at 65° C. for 30 minutes to 1 hour is optional. The filter is then preferably dried and subjected to autoradiography for sufficient time to visualize the positives on the X-ray film. Other known hybridization methods can also be employed.

The positive colonies are picked, grown in culture, and plasmid DNA isolated using standard procedures. The clones can then be verified by restriction analysis, hybridization analysis, or DNA sequencing.

Fragments of the proteins of the present invention which are capable of exhibiting biological activity are also encompassed by the present invention. Fragments of the protein maybe in linear form or they may be cyclized using known methods, for example, as described in H. U. Saragovi, et al., Bio/Technology 10, 773-778 (1992) and in R. S. McDowell, et al., J. Amer. Chem. Soc. 11.4 9245-9253 (1992), both of which are incorporated herein by reference. Such fragments maybe fused to carrier molecules such as immunoglobulins for many purposes, including increasing the valency of protein binding sites. For example, fragments of the protein may be fused through “linker” sequences to the Fc portion of an immunoglobulin. For a bivalent form of the protein, such a fusion could be to the Fc portion of an IgG molecule. Other immunoglobulin isotypes may also be used to generate such fusions. For example, a protein-IgM fusion would generate a decavalent form of the protein of the invention.

The present invention also provides both full-length and mature forms of the disclosed proteins. The full-length form of the such proteins is identified in the sequence listing by translation of the nucleotide sequence of each disclosed clone. The mature form(s) of such protein may be obtained by expression of the disclosed full-length polynucleotide (preferably those deposited with ATCC) in a suitable mammalian cell or other host cell. The sequence(s) of the mature form(s) of the protein may also be determinable from the amino acid sequence of the full-length form.

The present invention also provides genes corresponding to the polynucleotide sequences disclosed herein. “Corresponding genes” are the regions of the genome that are transcribed to produce the mRNAs from which cDNA polynucleotide sequences are derived and may include contiguous regions of the genome necessary for the regulated expression of such genes. Corresponding genes may therefore include but are not limited to coding sequences, 5′ and 3′ untranslated regions, alternatively spliced exons, introns, promoters, enhancers, and silencer or suppressor elements. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials. An “isolated gene” is a gene that has been separated from the adjacent coding sequences, if any, present in the genome of the organism from which the gene was isolated.

The chromosomal location corresponding to the polynucleotide sequences disclosed herein may also be determined, for example by hybridizing appropriately labeled polynucleotides of the present invention to chromosomes in situ. It may also be possible to determine the corresponding chromosomal location for a disclosed polynucleotide by identifying significantly similar nucleotide sequences in public databases, such as expressed sequence tags (ESTs), that have already been mapped to particular chromosomal locations. For at least some of the polynucleotide sequences disclosed herein, public database sequences having at least some similarity to the polynucleotide of the present invention have been listed by database accession number. Searches using the GenBank accession numbers of these public database sequences can then be performed at an Internet site provided by the National Center for Biotechnology Information having the address http://www.ncbi.nlm.nih.gov/UniGene/, in order to identify “UniGene clusters” of overlapping sequences. Many of the “UniGene clusters” so identified will already have been mapped to particular chromosomal sites.

Organisms that have enhanced, reduced, or modified expression of the gene(s) corresponding to the polynucleotide sequences disclosed herein are provided. The desired change in gene expression can be achieved through the use of antisense polynucleotides or ribozymes that bind and/or cleave the mRNA transcribed from the gene (Albert and Morris, 1994, Trends Pharmacol. Sci. 15(7): 250-254; Lavarosky et al., 1997, Biochem. Mol. Med. 62(1): 11_{—}22; and Hampel, 1998, Prog. Nucleic Acid Res. Mol. Biol. 58: 1-39; all of which are incorporated by reference herein). Transgenic animals that have multiple copies of the gene(s) corresponding to the polynucleotide sequences disclosed herein, preferably produced by transformation of cells with genetic constructs that are stably maintained within the transformed cells and their progeny, are provided. Transgenic animals that have modified genetic control regions that increase or reduce gene expression levels, or that change temporal or spatial patterns of gene expression, are also provided (see European Patent No. 0 649 464 B1, incorporated by reference herein). In addition, organisms are provided in which the gene(s) corresponding to the polynucleotide sequences disclosed herein have been partially or completely inactivated, through insertion of extraneous sequences into the corresponding gene(s) or through deletion of all or part of the corresponding gene(s). Partial or complete gene inactivation can be accomplished through insertion, preferably followed by imprecise excision, of transposable elements (Plasterk, 1992, Bioessays 14(9): 629_{—}633; Zwaal et al., 1993, Proc. Natl. Acad. Sci. USA 90(16): 7431_{—}7435; Clark et al., 1994, Proc. Natl. Acad. Sci. USA 91(2): 719-722; all of which are incorporated by reference herein), or through homologous recombination, preferably detected by positive/negative genetic selection strategies (Mansour et al., 1988, Nature 336: 348-352; U.S. Patent Nos. 5,464,764; 5,487,992; 5,627,059; 5,631,153; 5,614,396; 5,616,491; and 5,679,523; all of which are incorporated by reference herein). These organisms with altered gene expression are preferably eukaryotes and more preferably are mammals. Such organisms are useful for the development of non-human models for the study of disorders involving the corresponding gene(s), and for the development of assay systems for the identification of molecules that interact with the protein product(s) of the corresponding gene(s).

Where the protein of the presentinvention is membrane-bound (e.g., is a receptor), the present invention also provides for soluble forms of such protein. In such forms, part or all of the intracellular and transmembrane domains of the protein are deleted such that the protein is fully secreted from the cell in which it is expressed. The intracellular and transmembrane domains of proteins of the invention can be identified in accordance with known techniques for determination of such domains from sequence information. For example, the TopPredIH computer program can be used to predict the location of transmembrane domains in an amino acid sequence, domains which are described by the location of the center of the transmsmbrane domain, with at least ten transmembrane amino acids on each side of the reported central residue(s).

Proteins and protein fragments of the present invention include proteins with amino acid sequence lengths that are at least 25%(more preferably at least 50%, and most preferably at least 75%) of the length of a disclosed protein and have at least 60% sequence identity (more preferably, at least 75% identity; most preferably at least 90% or 95% identity) with that disclosed protein, where sequence identity is determined by comparing the amino acid sequences of the proteins when aligned so as to maximize overlap and identity while minimizing sequence gaps. Also included in the present invention are proteins and protein fragments that contain a segment preferably comprising 8 or more (more preferably 20 or more, most preferably 30 or more) contiguous amino acids that shares at least 75% sequence identity (more preferably, at least 85% identity; most preferably at least 95% identity) with any such segment of any of the disclosed proteins.

In particular, sequence identity may be determined using WU-BLAST (Washington University BLAST) version 2.0 software, which builds upon WU-BLAST version 1.4, which in turn is based on the public domain NCBI-BLAST version 1.4 (Altschul and Gish, 1996, Local alignment statistics, Doolittle ed., Methods in Enzymology 266: 460-480; Altschul et al., 1990, Basic local alignment search tool, Journal of Molecular Biology 215: 403-410; Gish and States, 1993, Identification of protein coding regions by database similarity search, Nature Genetics 3: 266-272; Karlin and Altschul, 1993, Applications and statistics for multiple high-scoring segments in molecular sequences, Proc. Natl. Acad. Sci. USA 90: 5873_{—}5877; all of which are incorporated by reference herein). WU-BLAST version 2.0 executable programs for several UNIX platforms can be downloaded from ftp:/blast.wustl.edu/blastlexecutables. The complete suite of search programs (BLASTP, BLASTN, BLASTX, TBLASTN, and TBLASTX) is provided at that site, in addition to several support programs. WU-BLAST 2.0 is copyrighted and may not be sold or redistributed in any form or manner without the express written consent of the author; but the posted executables may otherwise be freely used for commercial, nonprofit, or academic purposes. In all search programs in the suite —BLASTP, BLASTN, BLASTX, TBLASTN and TBLASTX—the gapped alignment routines are integral to the database search itself, and thus yield much better sensitivity and selectivity while producing the more easily interpreted output. Gapping can optionally be turned off in all of these programs, if desired. The default penalty (Q) for a gap of length one is Q=9 for proteins and BLASTP, and Q=10 for BLASTN, but may be changed to any integer value including zero, one through eight, nine, ten, eleven, twelve through twenty, twenty-one through fifty, fifty-one through one hundred, etc. The default per-residue penalty for extending a gap (R) is R=2 for proteins and BLASTP, and R=10 for BLASTN, but may be changed to any integer value including zero, one, two, three, four, five, six, seven, eight, nine, ten, eleven, twelve through twenty, twenty-one through fifty, fifty-one through one hundred, etc. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Species homologues of the disclosed polynucleotides and proteins are also provided by the present invention. As used herein, a “species homologue” is a protein or polynucleotide with a different species of origin from that of a given protein or polynucleotide, but with significant sequence similarity to the given protein or polynucleotide. Preferably, polynucleotide species homologues have at least 60% sequence identity (more preferably, at least 75% identity; most preferably at least 90% identity) with the given polynucleotide, and protein species homologues have at least 30% sequence identity (more preferably, at least 45% identity; most preferably at least 60% identity) with the given protein, where sequence identity is determined by comparing the nucleotide sequences of the polynucleotides or the amino acid sequences of the proteins when aligned so as to maximize overlap and identity while minimizing sequence gaps. Species homologues may be isolated and identified by making suitable probes or primers from the sequences provided herein and screening a suitable nucleic acid source from the desired species. Preferably, species homologues are those isolated from ma:malian species. Most p referably, species homologues are those isolated from certain mammalian species such as, for example, Pan troglodytes, Gorilla gorilla, Pongo pygmaeus, Hylobates concolor, Macaca muZatta, Papio papio, Papio hamadryas, Cercopithecus aethiops, Cebus capucinus, Aotus trivirgatus, Sanguinus oedipus, Microcebus murinus, Mus musculus, Rattus norvegicus, Cricetulusgriseus,Felis catus,Mustela vison, Canisfamiliaris, Oryctolagus cuniculus, Bos taurus, Ovis aries, Sus scrofa, and Equus caballus, for which genetic maps have been created allowing the identification of syntenic relationships between the genomic organization of genes in one species and the genomic organization of the related genes in another species (O'Brien and Seuanez, 1988, Ann. Rev. Genet. 22: 323351; O'Brien et al., 1993, Nature Genetics 3:103-112; Johansson et al., 1995, Genomics 25: 682-690; Lyons et al., 1997, Nature Genetics 15: 47-56; O'Brien et al., 1997, Trends in Genetics 13(10):393-399; Carver and Stubbs, 1997, Genome Research 7:1123-1137; all of which are incorporated by reference herein).

The invention also encompasses allelic variants of the disclosed polynucleotides or proteins; that is, naturally-occurring alternative forms of the isolated polynucleotides which also encode proteins which are identical or have significantly similar sequences to those encoded by the disclosed polynucleotides. Preferably, allelic variants have at least 60% sequence identity (more preferably, at least 75% identity; most preferably at least 90% identity) with the given polynucleotide, where sequence identity is determined by comparing the nucleotide sequences of the polynucleotides when aligned so as to maximize overlap and identity while minimizing sequence gaps. Allelic variants may be isolated and identified by making suitable probes or primers from the sequences provided herein and screening a suitable nucleic acid source from individuals of the appropriate species.

The invention also includes polynucleotides with sequences complementary to those of the polynucleotides disclosed herein.

The present invention also includes polynucleotides that hybridize under reduced stringency conditions, more preferably stringent conditions, and most preferably highly stringent conditions, to polynucleotides described herein. Examples of stringency conditions are shown in the table below: highly stringent conditions are those that are at least as stringent as, for example, conditions A-F; stringent conditions are at least as stringent as, for example, conditions G-L; and reduced stringency conditions are at least as stringent as, for example, conditions M-R.

| Strin- | | | | | | |
|---|---|---|---|---|---|---|
| gency | Poly- | Hybrid | Hybridization | | Wash | |
| Con- | nucleotide | Length | Temperature and | | Temperature | |
| dition | Hybrid | (bp)^{‡} | Buffer^{†} | | and Buffer^{†} | |
| A | DNA:DNA | ≧50 | 65° C.; | 1 × SSC | 65° C.; | 0.3 × SSC |
| | | | -or- | | | |
| | | | 42° C.; | 1 × SSC, | | |
| | | | 50% | formamide | | |
| B | DNA:DNA | <50 | T_{B}*; | 1 × SSC | T_{B}*; | 1 × SSC |
| C | DNA:RNA | ≧50 | 67° C.; | 1 × SSC | 67° C.; | 0.3 × SSC |
| | | | -or- | | | |
| | | | 45° C.; | 1 × SSC, | | |
| | | | 50% | formamide | | |
| D | DNA:RNA | <50 | T_{D}*; | 1 × SSC | T_{D}*; | 1 × SSC |
| E | RNA:RNA | ≧50 | 70° C.; | 1 × SSC | 70° C.; | 0.3 × SSC |
| | | | -or- | | | |
| | | | 50° C.; | 1 × SSC, | | |
| | | | 50% | formamide | | |
| F | RNA:RNA | <50 | T_{G}*; | 1 × SSC | T_{F}*; | 1 × SSC |
| G | DNA:DNA | ≧50 | 65° C.; | 4 × SSC | 65° C.; | 1 × SSC |
| | | | -or- | | | |
| | | | 42° C.; | 4 × SSC, | | |
| | | | 50% | formamide | | |
| H | DNA:DNA | <50 | T_{H}*; | 4 × SSC | T_{H}*; | 4 × SSC |
| I | DNA:RNA | ≧50 | 67° C.; | 4 × SSC | 67° C.; | 1 × SSC |
| | | | -or- | | | |
| | | | 45° C.; | 4 × SSC, | | |
| | | | 50% | formamide | | |
| J | DNA:RNA | <50 | T_{J}*; | 4 × SSC | T_{J}*; | 4 × SSC |
| K | RNA:RNA | ≧50 | 70° C.; | 4 × SSC | 67° C.; | 1 × SSC |
| | | | -or- | | | |
| | | | 50° C.; | 4 × SSC, | | |
| | | | 50% | formamide | | |
| L | RNA:RNA | <50 | T_{L}*; | 2 × SSC | | |
| M | DNA:DNA | ≧50 | 50° C.; | 4 × SSC | 50° C.; | 2 × SSC |
| | | | -or- | | | |
| | | | 40° C.; | 6 × SSC, | | |
| | | | 50% | formamide | | |
| N | DNA:DNA | <50 | T_{N}*; | 6 × SSC | T_{N}*; | 6 × SSC |
| O | DNA:RNA | ≧50 | 55° C.; | 4 × SSC | 55° C.; | 2 × SSC |
| | | | -or- | | | |
| | | | 42° C.; | 6 × SSC, | | |
| | | | 50% | formamide | | |
| P | DNA:RNA | <50 | T_{P}*; | 6 × SSC | T_{P}*; | 6 × SSC |
| Q | RNA:RNA | ≧50 | 60° C.; | 4 × SSC | 60° C.; | 2 × SSC |
| | | | -or- | | | |
| | | | 45° C.; | 6 × SSC, | | |
| | | | 50% | formamide | | |
| R | RNA:RNA | <50 | T_{R}*; | 4 × SSC | T_{R}*; | 4 × SSC |
| ^{‡}The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | | | |
| ^{†}SSPE(1 × SSPE is 0.15M NaCl, 10 mM NaH₂PO₄, 1.25 mM EDTA, pH 7.4) can be substituted for SSC (1 × SSC is 0.15M NaCl and 15 mM sodium citrate) in the hybridization and wash buffers; washes are performed for 15 minutes after hybridization is complete. | | | | | | |
| *T_{B}-T_{R}The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C | | | | | | |
| # bases). For hybrids between 18 and 49 base pairs in length, Tₘ(° C.) 81.5 + 16.6(log₁₀[Na⁺]) + 0.41(% G + C) − (600/N), where N is the number of bases in the hybrid, and [Na⁺] is the concentration of sodium ions in the hybridization buffer ([Na⁺] for 1 × SSC = 0.165M). | | | | | | |

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F.M. Ausubel et al., eds., John Wiley & Sons, Inc., sections 2.10 and 6.3_{—}6.4, incorporated herein by reference.

Preferably, each such hybridizing polynucleotide has a length that is at least 25%(more preferably at least 50%, and most preferably at least 75%) of the length of the polynucleotide of the present invention to which it hybridizes, and has at least 60% sequence identity (more preferably, at least 75% identity; most preferably at least 90% or 95% identity) with the polynucleotide of the present invention to which it hybridizes, where sequence identity is determined by comparing the sequences of the hybridizing polynucleotides when aligned so as to maximize overlap and identity while minimizing sequence gaps.

The isolated polynucleotide of the invention may be operably linked to an expression control sequence such as the pMT2 or pED expression vectors disclosed in Kaufman et al., Nucleic Acids Res. 19, 4485-4490 (1991), in order to produce the protein recombinantly. Many suitable expression control sequences are known in the art. General methods of expressing recombinant proteins are also known and are exemplified in R. Kaufman, Methods in Enzymology 185, 537-566 (1990). As defined herein “operably linked” means that the isolated polynucleotide of the invention and an expression control sequence are situated within a vector or cell in such a way that the protein is expressed by a host cell which has been transformed (transfected) with the ligated polynucleotide/expression control sequence.

A number of types of cells may act as suitable host cells for expression of the protein. Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

Alternatively, it may be possible to produce the protein in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein. Such covalent attachments may be accomplished using known chemical or enzymatic methods.

The protein may also be produced by operably linking the isolated polynucleotide of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac® kit), and such methods are well known in the art, as described in Summers and Smith, Texas Agricultural Experiment Station Bulletin No. 1555 (1987), incorporated herein by reference. As used herein, an insect cell capable of expressing a polynucleotide of the present invention is “transformed.”

The protein of the invention may be prepared by culturing transformed host cells under culture conditions suitable to express the recombinant protein. The resulting expressed protein may then be purified from such culture (i.e., from culture medium or cell extracts) using known purification processes, such as gel filtration and ion exchange chromatography. The purification of the protein may also include an affinity column containing agents which will bind to the protein; one or more column steps over such affinity resins as concanavalin A-agarose, heparin-toyopearl® or Cibacrom blue 3GA Sepharose®; one or more steps involving hydrophobic interaction chromatography using such resins as phenyl ether, butyl ether, or propyl ether; or immunoaffinity chromatography.

Alternatively, the protein of the invention may also be expressed in a form which will facilitate purification. For example, it may be expressed as a fusion protein, such as those of maltose binding protein (MBP), glutathione-S-transferase (GST) or thioredoxin (TRX). Kits for expression and purification of such fusion proteins are commercially available from New England BioLabs (Beverly, Mass.), Pharmacia (Piscataway, N.J.) and Invitrogen Corporation (Carlsbad, Calif.), respectively. The protein can also be tagged with an epitope and subsequently purified by using a specific antibody directed to such epitope. One such epitope (“Flag”) is commercially available from the Eastman Kodak Company (New Haven, Conn.).

Finally, one or more reverse-phase high performance liquid chromatography (RP-HPLC) steps employing hydrophobic RP-HPLC media, e.g., silica gel having pendant methyl or other aliphatic groups, can be employed to further purify the protein. Some or all of the foregoing purification steps, in various combinations, can also be employed to provide a substantially homogeneous isolated recombinant protein. The protein thus purified is substantially free of other mammalian proteins and is defined in accordance with the present invention as an “isolated protein.”

The protein of the invention may also be expressed as a product of transgenic animals, e.g., as a component of the milk of transgenic cows, goats, pigs, or sheep which are characterized by somatic or germ cells containing a nucleotide sequence encoding the protein.

The protein may also be produced by known conventional chemical synthesis. Methods for constructing the proteins of the present invention by synthetic means are known to those skilled in the art. The synthetically-constructed protein sequences, by virtue of sharing primary, secondary or tertiary structural and/or conformational characteristics with proteins may possess biological properties in common therewith, including protein activity. Thus, they may be employed as biologically active or immunological substitutes for natural, purified proteins in screening of therapeutic compounds and in immunological processes for the development of antibodies.

The proteins provided herein also include proteins characterized by amino acid sequences similar to those of purified proteins but into which modification are naturally provided or deliberately engineered. For example, modifications in the peptide or DNA sequences can be made by those skilled in the art using known techniques. Modifications of interest in the protein sequences may include the alteration, substitution, replacement, insertion or deletion of a selected amino acid residue in the coding sequence. For example, one or more of the cysteine residues may be deleted or replaced with another amino acid to alter the conformation of the molecule. Techniques for such alteration, substitution, replacement, insertion or deletion are well known to those skilled in the art (see, e.g., U.S. Pat. No. 4,518,584). Preferably, such alteration, substitution, replacement, insertion or deletion retains the desired activity of the protein.

Other fragments and derivatives of the sequences of proteins which would be expected to retain protein activity in whole or in part and may thus be useful for screening or other immunological methodologies may also be easily made by those skilled in the art given the disclosures herein. Such modifications are believed to be encompassed by the present invention.

Uses and Biological Activity

The polynucleotides and proteins of the present invention are expected to exhibit one or more of the uses or biological activities (including those associated with assays cited herein) identified below. Uses or activities described for proteins of the present invention may be provided by administration or use of such proteins or by administration or use of polynucleotides encoding such proteins (such as, for example, in gene therapies or vectors suitable for introduction of DNA).

Research Uses and Utilities

The polynucleotides provided by the present invention can be used by the research community for various purposes. The polynucleotides can be used to express recombinant protein for analysis, characterization or therapeutic use; as markers for tissues in which the corresponding protein is preferentially expressed (either constitutively or at a particular stage of tissue differentiation or development or in disease states); as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “subtract-out” known sequences in the process of discovering other novel polynucleotides; for selecting and making oligomers for attachment to a “gene chip” or other support, including for examination of expression patterns; to raise anti-protein antibodies using DNA immunization techniques; and as an antigen to raise anti-DNA antibodies or elicit another immune response. Where the polynucleotide encodes a protein whichbinds or potentially binds to another protein (such as, for example, in a receptor-ligand interaction), the polynucleotide can also be used in interaction trap assays (such as, for example, those described in Gyuris et al., 1993, Cell 75: 791_{—}803 and in Rossi et al., 1997, Proc. Natl. Acad. Sci. USA 94: 8405_{—}8410, all of which are incorporated by reference herein) to identify polynucleotides encoding the other protein with which binding occurs or to identify inhibitors of the binding interaction.

The proteins provided by the present invention can similarly be used in assay to determine biological activity, including in a panel of multiple proteins for high-throughput screening; to raise antibodies or to elicit another immune response; as a reagent (including the labeled reagent) in assays designed to quantitatively determine levels of the protein (or its receptor) in biological fluids; as markers for tissues in which the corresponding protein is preferentially expressed (either constitutively or at a particular stage of tissue differentiation or development or in a disease state); and, of course, to isolate correlative receptors or ligands. Where the protein binds or potentially binds to another protein (such as, for example, in a receptor-ligand interaction), the protein can be used to identify the other protein with which binding occurs or to identify inhibitors of the binding interaction. Proteins involved in these binding interactions can also be used to screen for peptide or small molecule inhibitors or agonists of the binding interaction.

Any or all of these research utilities are capable of being developed into reagent grade or kit format for commercialization as research products.

Methods for performing the uses listed above are well known to those skilled in the art. References disclosing such methods include without limitation “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

Nutritional Uses

Polynucleotides and proteins of the present invention can also be used as nutritional sources or supplements. Such uses include without limitation use as a protein or amino acid supplement, use as a carbon source, use as a nitrogen source and use as a source of carbohydrate. In such cases the protein or polynucleotide of the invention can be added to the feed of a particular organism or can be administered as a separate solid or liquid preparation, such as in the form of powder, pills, solutions, suspensions or capsules. In the case of microorganisms, the protein or polynucleotide of the invention can be added to the medium in or on which the microorganism is cultured.

Cytokine and Cell Proliferation/Differentiation Activity

A protein of the present invention may exhibit cytokine, cell proliferation (either inducing or inhibiting) or cell differentiation (either inducing or inhibiting) activity or may induce production of other cytokines in certain cell populations. Many protein factors discovered to date, including all known cytokines, have exhibited activity in one or more factor-dependent cell proliferation assays, and hence the assays serve as a convenient confirmation of cytokine activity. The activity of a protein of the present invention is evidenced by any one of a number of routine factor dependent cell proliferation assays for cell lines including, without limitation, 32D, DA2, DA1G, T10, B9, B9/11, BaF3, MC9/G, M+(preB M+), 2E8, RB5, DA1, 123, T1165, HT2, CTLL2, TF-1, Mo7e and CMK. The activity of a protein of the invention may, among other means, be measured by the following methods: Assays for T-cell or thymocyte proliferation include without limitation those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Bertagnolli et al., J. Immunol. 145:1706-1712, 1990; Bertagnolli et al., Cellular Immunology 133:327-341, 1991; Bertagnolli, et al., J. Immunol. 149:3778-3783, 1992; Bowman et al., J. Immunol. 152: 1756-1761, 1994.

Assays for cytokine production and/or proliferation of spleen cells, lymph node cells or thymocytes include, without limitation, those described in: Polyclonal T cell stimulation, Kruisbeek, A. M. and Shevach, E. M. In Current Protocols in Immunology. J. E. e.a. Coligan eds. Vol 1 pp. 3.12.1-3.12.14, John Wiley and Sons, Toronto. 1994; and Measurement of mouse and human Interferon γ, Schreiber, R. D. In Current Protocols in Immunology. J. E. e.a. Coligan eds. Vol 1 pp. 6.8.1-6.8.8, John Wiley and Sons, Toronto. 1994.

Assays for proliferation and differentiation of hematopoietic and lymphopoietic cells include, without limitation, those described in: Measurement of Human and Murine Interleukin 2 and Interleukin 4, Bottomly, K., Davis, L. S. and Lipsky, P. E. In Current Protocols in Immunology. J. E. e.a. Coligan eds. Vol 1 pp. 6.3.1-6.3.12, John Wiley and Sons, Toronto. 1991; deVries et al., J. Exp. Med. 173:1205-1211, 1991; Moreau et al., Nature 336:690-692, 1988; Greenberger et al., Proc. Natl. Acad. Sci. U.S.A. 80:2931-2938, 1983; Measurement of mouse and human interleukin 6- Nordan, R. In Current Protocols in Immunology. J. E. e.a. Coligan eds. Vol 1 pp. 6.6.1-6.6.5, John Wiley and Sons, Toronto. 1991; Smith et al., Proc. Natl. Acad. Sci. U.S.A. 83:1857-1861, 1986; Measurement of human Interleukin 11- Bennett, F., Giannotti, J., Clark, S. C. and Turner, K. J. In Current Protocols in Immunology. J. E. e.a. Coligan eds. Vol 1 pp. 6.15.1 John Wiley and Sons, Toronto. 1991; Measurement of mouse and human Interleukin 9- Ciarletta, A., Giannotti, J., Clark, S. C. and Turner, K. J. In Current Protocols in Immunology. J. E. e.a. Coligan eds. Vol 1 pp. 6.13.1, John Wiley and Sons, Toronto. 1991. Assays for T-cell clone responses to antigens (which will identify, among others, proteins that affect APC-T cell interactions as well as direct T-cell effects by measuring proliferation and cytokine production) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function; Chapter 6, Cytokines and their cellular receptors; Chapter 7, Immunologic studies in Humans); Weinberger et al., Proc. Natl. Acad. Sci. USA 77:6091-6095,1980; Weinberger et al., Eur. J. Immun. 11:405-411, 1981; Takai et al., J. Immunol. 137:3494-3500, 1986; Takai et al., J. Immunol. 140:508-512, 1988.

Immune Stimulating or Suppressing Activitv

A protein of the present invention may also exhibit immune stimulating or immune suppressing activity, including without limitation the activities for which assays are described herein. A protein may be useful in the treatment of various immune deficiencies and disorders (including severe combined immnunodeficiency (SCID)), e.g., in regulating (up or down) growth and proliferation of T and/or B lymphocytes, as well as effecting the cytolytic activity of NK cells and other cell populations. These immune deficiencies may be genetic or be caused by viral (e.g., HIV) as well as bacterial or fungal infections, or may result from autoimmune disorders. More specifically, infectious diseases causes by viral, bacterial, fungal or other infection may be treatable using a protein of the present invention, including infections by HIV, hepatitis viruses, herpesviruses, mycobacteria, Leishmania spp., malaria spp. and various fungal infections such as candidiasis. Of course, in this regard, a protein of the present invention may also be useful where a boost to the immune system generally may be desirable, i.e., in the treatment of cancer. Autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitis, myasthenia gravis, graft-versus-host disease and autoimmune inflammatory eye disease.

Such a protein of the present invention may also to be useful in the treatment of allergic reactions and conditions, such as asthma (particularly allergic asthma) or other respiratory problems. Other conditions, in which immune suppression is desired (including, for example, organ transplantation), may also be treatable using a protein of the present invention.

Using the proteins of the invention it may also be possible to regulate immune responses in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or anergy in T cells, is distinguishable from immunosuppression in that it is generally antigen-specific and persists after exposure to the tolerizing agent has ceased. Operationally, tolerance can be demonstrated by the lack of a T cell response upon reexposure to specific antigen in the absence of the tolerizing agent.

Down regulating or preventing one or more antigen functions (including without limitation B lymphocyte antigen functions (such as, for example, B7)), e.g., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a molecule which inhibits or blocks interaction of a B7 lymphocyte antigen with its natural ligand(s) on immune cells (such as a soluble, monomeric form of a peptide having B7-2 activity alone or in conjunction with a monomeric form of a peptide having an activity of another B lymphocyte antigen (e.g., B7-1, B7-3) or blocking antibody), prior to transplantation can lead to the binding of the molecule to the natural ligand(s) on the immune cells without transmitting the corresponding costimulatory signal. Blocking B lymphocyte antigen function in this matter prevents cytokine synthesis by immune cells, such as T cells, and thus acts as an immunosuppressant. Moreover, the lack of costirnulation may also be sufficient to anergize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of a combination of B lymphocyte antigens.

The efficacy of particular blocking reagents in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257:789-792 (1992) and Turka et al., Proc. Natl. Acad. Sci USA, 89:11102-11105 (1992). In addition, murine models of GVHD (see Paul ed., Fundamental Immunology, Raven Press, New York, 1989, pp. 846-847) can be used to determine the effect of blocking B lymphocyte antigen function in vivo on the development of that disease.

Blocking antigen function may also be therapeutically useful for treating autoimmune diseases. Many autoimnuune disorders are the result of inappropriate activation of T cells that are reactive against self tissue and which promote the production of cytokines and autoantibodies involved in the pathology of the diseases. Preventing the activation of autoreactive T cells may reduce or eliminate disease symptoms. Administration of reagents which block costimulation of T cells by disrupting receptor:ligand interactions of B lymphocyte antigens can be used to inhibit T cell activation and prevent production of autoantibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of human autoimmune diseases. Examples include murine experimental autoimmune encephalitis, systemic lupus erythmatosis in MRL/lpr/Zpr mice or NZB hybrid mice, murine autoimmune collagen arthritis, diabetes mellitus in NOD mice and BB rats, and murine experimental myasthenia gravis (see Paul ed., Fundamental Immunology, Raven Press, New York, 1989, pp. 840856).

Upregulation of an antigen function (preferably a B lymphocyte antigen function), as a means of up regulating immune responses, may also be useful in therapy. Upregulation of immune responses may be in the form of enhancing an existing immune response or eliciting an initial immune response. For example, enhancing an immune response through stimulating B lymphocyte antigen function may be useful in cases of viral infection. In addition, systemic viral diseases such as influenza, the common cold, and encephalitis might be alleviated by the administration of stimulatory forms of B lymphocyte antigens systemically.

Alternatively, anti-viral immune responses maybe enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. Another method of enhancing anti-viral immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

In another application, up regulation or enhancement of antigen function (preferably B lymphocyte antigen function) may be useful in the induction of tumor immunity. Tumor cells (e.g., sarcoma, melanoma, lymphoma, leukemia, neuroblastoma, carcinoma) transfected with a nucleic acid encoding at least one peptide of the present invention can be administered to a subject to overcome tumor-specific tolerance in the subject. If desired, the tumor cell can be transfected to express a combination of peptides. For example, tumor cells obtained from a patient can be transfected ex vivo with an expression vector directing the expression of a peptide having B7-2-like activity alone, or in conjunction with a peptide having B7-1-like activity and/or B7-3-like activity. The transfected tumor cells are returned to the patient to result in expression of the peptides on the surface of the transfected cell. Alternatively, gene therapy techniques can be used to target a tumor cell for transfection in vivo.

The presence of the peptide of the present invention having the activity of a B lymphocyte antigen(s) on the surface of the tumor cell provides the necessary costimulation signal to T cells to induce a T cell mediated immune response against the transfected tumor cells. In addition, tumor cells which lack MHC class I or MHC class II molecules, or which fail to reexpress sufficient amounts of MHC class I or MHC class II molecules, can be transfected with nucleic acid encoding all or a portion of (e.g., a cytoplasmic-domain truncated portion) of an MHC class I α chain protein and β₂ microglobulin protein or an MHC class II α chain protein and an MHC class II β chain protein to thereby express MHC class I or MHC class II proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject.

The activity of a protein of the invention may, among other means, be measured by the following methods:

Suitable assays for thymocyte or splenocyte cytotoxicity include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500,1986; Takai et al., J. Immunol. 140:508-512, 1988; Herrmann et al., Proc. Natl. Acad. Sci. USA 78:2488-2492, 1981; Herrmann et al., J. Immunol. 128:1968-1974, 1982; Handa et al., J. Immunol. 135:1564-1572,1985; Takai et al., J. Immunol. 137:3494-3500,1986; Bowmanet al., J. Virology 61:1992-1998; Takai et al., J. Immunol. 140:508-512, 1988; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, proteins that modulate T-cell dependent antibody responses and that affect Thl/Th2 profiles) include, without limitation, those described in: Maliszewski, J. Immunol. 144:3028-3033, 1990; and Assays for B cell function: In vitro antibody production, Mond, J. J. and Brunswick, M. In Current Protocols in Immunology. J. E. e.a. Coligan eds. Vol 1 pp. 3.8.1-3.8.16, John Wiley and Sons, Toronto. 1994.

Mixed lymphocyte reaction (MLR) assays (which will identify, among others, proteins that generate predominantly Thl and CTL responses) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Takai et al., J. Immunol. 140:508-512, 1988; Bertagnolli et al., J. Immunol. 149:3778-3783, 1992. Dendritic cell-dependent assays (which will identify, among others, proteins expressed by dendritic cells that activate naive T-cells) include, without limitation, those described in: Guery et al., J. Immunol. 134:536-544, 1995; Inaba et al., Journal of Experimental Medicine 173:549-559, 1991; Macatonia et al., Journal of Immunology 154:5071-5079, 1995; Porgador et al., Journal of Experimental Medicine 182:255-260, 1995; Nair et al., Journal of Virology 67:4062-4069,1993; Huang et al., Science 264:961-965,1994; Macatonia et al., Journal of Experimental Medicine 169:1255-1264, 1989; Bhardwaj et al., Journal of Clinical Investigation 94:797-807,1994; and Inaba et al., Journal of Experimental Medicine 172:631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, Journal of Immunology 145:4037-4045, 1990; Zamai et al., Cytometry 14:891-897, 1993; Gorczyca et al., International Journal of Oncology 1:639648, 1992.

Assays for proteins that influence early steps of T-cell commitment and development include, without limitation, those described in: Antica et al., Blood 84:111-117, 1994; Fine et al., Cellular Immunology 155:111-122, 1994; Galy et al., Blood 85:2770-2778, 1995; Toki et al., Proc. Nat. Acad Sci. USA 88:7548-7551, 1991.

Hematopoiesis Regulating Activity

A protein of the present invention may be useful in regulation of hematopoiesis and, consequently, in the treatment of myeloid or lymphoid cell deficiencies. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional CSF activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in-vivo or ex-vivo (i.e., in conjunction with bone marrow transplantation or with peripheral progenitor cell transplantation (homologous or heterologous)) as normal cells or genetically manipulated for gene therapy.

The activity of a protein of the invention may, among other means, be measured by the following methods:

Suitable assays for proliferation and differentiation of various hematopoietic lines are cited above.

Assays for embryonic stem cell differentiation (which will identify, among others, proteins that influence embryonic differentiation hematopoiesis) include, without limitation, those described in: Johansson et al. Cellular Biology 15:141-151, 1995; Keller et al., Molecular and Cellular Biology 13:473-486, 1993; McClanahan et al., Blood 81:2903-2915, 1993.

Assays for stem cell survival and differentiation (which will identify, among others, proteins that regulate lympho-hematopoiesis) include, without limitation, those described in: Methylcellulose colony forming assays, Freshney, M. G. In Culture ofHematopoietic Cells. R. I. Freshney, et al. eds. Vol pp. 265-268, Wiley-Liss, Inc., New York, NY. 1994; Hirayama et al., Proc. Natl. Acad. Sci. USA 89:5907-5911, 1992; Primitive hematopoietic colony forming cells with high proliferative potential, McNiece, I. K. and Briddell, R. A. In Culture of Hematopoietic Cells. R. I. Freshney, et al. eds. Vol pp. 23-39, Wiley-Liss, Inc., New York, NY. 1994; Neben et al., Experimental Hematology 22:353-359, 1994; Cobblestone area forming cell assay, Ploemacher, R. E. In Culture of Hematopoietic Cells. R. I. Freshney, et al. eds. Vol pp.1-21, Wiley-Liss, Inc., New York, N.Y. 1994; Long term bone marrow cultures in the presence of stromal cells, Spooncer, E., Dexter, M. and Allen, T. In Culture of Hematopoietic Cells. R. I. Freshney, et al. eds. Vol pp. 163-179, Wiley-Liss, Inc., New York, N.Y. 1994; Long term culture initiating cell assay, Sutherland, H.J. In Culture of Hematopoietic Cells. R. I. Freshney, et al. eds. Vol pp. 139-162, Wiley-Liss, Inc., New York, N.Y. 1994.

Tissue Growth Activity A protein of the present invention also may have utility in compositions used for bone, cartilage, tendon, ligament and/or nerve tissue growth or regeneration, as well as for wound healing and tissue repair and replacement, and in the treatment of burns, incisions and ulcers.

A protein of the present invention, which induces cartilage and/or bone growth in circumstances where bone is not normally formed, has application in the healing of bone fractures and cartilage damage or defects in humans and other animals. Such a preparation employing a protein of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

A protein of this invention may also be used in the treatment of periodontal disease, and in other tooth repair processes. Such agents may provide an environment to attract bone-forming cells, stimulate growth of bone-forming cells or induce differentiation of progenitors of bone-forming cells. A protein of the invention may also be useful in the treatment of osteoporosis or osteoarthritis, such as through stimulation of bone and/or cartilage repair orbyblocking inflammation or processes of tissue destruction (collagenase activity, osteoclast activity, etc.) mediated by inflammatory processes.

Another category of tissue regeneration activity that may be attributable to the protein of the present invention is tendon/ligament formation. A protein of the present invention, which induces tendon/ligament-like tissue or other tissue formation in circumstances where such tissue is not normally formed, has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide an environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions of the invention may also be useful in the treatment of tendinitis, carpal tunnel syndrome and other tendon or ligament defects. The compositions may also include an appropriate matrix and/or sequestering agent as a carrier as is well known in the art.

The protein of the present invention may also be useful for proliferation of neural cells and for regeneration of nerve and brain tissue, i.e. for the treatment of central and peripheral nervous system diseases and neuropathies, as well as mechanical and traumatic disorders, which involve degeneration, death or trauma to neural cells or nerve tissue. More specifically, a protein may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the present invention include mechanical and traumatic disorders, such as spinal cord disorders, head trauma and cerebrovascular diseases such as stroke. Peripheral neuropathies resulting from chemotherapy or other medical therapies may also be treatable using a protein of the invention.

Proteins of the invention may also be useful to promote better or faster closure of non-healing wounds, including without limitation pressure ulcers, ulcers associated with vascular insufficiency, surgical and traumatic wounds, and the like.

It is expected that a protein of the present invention may also exhibit activity for generation or regeneration of other tissues, such as organs (including, for example, pancreas, liver, intestine, kidney, skin, endothelium), muscle (smooth, skeletal or cardiac) and vascular (including vascular endothelium) tissue, or for promoting the growth of cells comprising such tissues. Part of the desired effects may be by inhibition or modulation of fibrotic scarring to allow normal tissue to regenerate. A protein of the invention may also exhibit angiogenic activity.

A protein of the present invention may also be useful for gut protection or regeneration and treatment of lung or liver fibrosis, reperfusion injury in various tissues, and conditions resulting from systemic cytokine damage.

A protein of the present invention may also be useful for promoting or inhibiting differentiation of tissues described above from precursor tissues or cells; or for inhibiting the growth of tissues described above.

The activity of a protein of the invention may, among other means, be measured by the following methods:

Assays for tissue generation activity include, without limitation, those described in: International Patent Publication No. WO95/16035 (bone, cartilage, tendon);

International Patent Publication No. WO95/05846 (nerve, neuronal); International Patent Publication No. WO91/07491 (skin, endothelium).

Assays for wound healing activity include, without limitation, those described in: Winter, Epidermal Wound Healing, pps. 71-112 (Maibach, HI and Rovee, DT, eds.), Year Book Medical Publishers, Inc., Chicago, as modified by Eaglstein and Mertz, J. Invest. Dermatol 71:382-84 (1978).

Activin/Inhibin Activity

A protein of the present invention may also exhibit activin- or inhibin-related activities. Inhibins are characterized by their ability to inhibit the release of follicle stimulating hormone (FSH), while activins and are characterized by their ability to stimulate the release of follicle stimulating hormone (FSH). Thus, a protein of the present invention, alone or in heterodimers with a member of the inhibin cc family, may be useful as a contraceptive based on the ability of inhibins to decrease fertility in female mammals and decrease spermatogenesis in male mammals. Administration of sufficient amounts of other inhibins can induce infertility in these mammals. Alternatively, the protein of the invention, as a homodimer or as a heterodimer with other protein subunits of the inhibin-P group, may be useful as a fertility inducing therapeutic, based upon the ability of activin molecules in stimulating PSH release from cells of the anterior pituitary. See, for example, U.S. Pat. No. 4,798,885. A protein of the invention may also be useful for advancement of the onset of fertility in sexually immature mammals, so as to increase the lifetime reproductive performance of domestic animals such as cows, sheep and pigs. The activity of a protein of the invention may, among other means, be measured by the following methods:

Assays for activin/inhibin activity include, without limitation, those described in: Vale et al., Endocrinology 91:562-572, 1972; Ling et al., Nature 321:779-782, 1986; Vale et al., Nature 321:776-779, 1986; Mason et al., Nature 318:659-663, 1985; Forage et al., Proc. Natl. Acad. Sci. USA 83:3091-3095, 1986.

Chemotactic/Chemokinetic Activitv

A protein of the present invention may have chemotactic or chemokinetic activity (e.g., act as a chemokine) for mammalian cells, including, for example, monocytes, fibroblasts, neutrophils, T-cells, mast cells, eosinophils, epithelial and/or endothelial cells. Chemotactic and chemokinetic proteins can be used to mobilize or attract a desired cell population to a desired site of action. Chemotactic or chemokinetic proteins provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention may, among other means, be measured by the following methods:

Assays for chemotactic activity (which will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: Current Protocols in Immunology, Ed by J. E. Coligan, A. M. Kruisbeek, D. H. Margulies, E. M. Shevach, W.Strober, Pub. Greene Publishing Associates and Wiley-Interscience (Chapter 6.12, Measurement of alpha and beta Chemokines 6.12.1-6.12.28; Taub et al. J. Clin. Invest. 95:1370-1376, 1995; Lind et al. APMIS 103:140-146, 1995; Muller et al Eur. J. Immunol. 25: 17441748; Gruber et al. J. of Immunol. 152:5860-5867, 1994; Johnston et al. J. of Immunol. 153: 1762-1768, 1994.

Hemostatic and Thrombolvtic Activitv

A protein of the invention may also exhibit hemostatic or thrombolytic activity. As a result, such a protein is expected to be useful in treatment of various coagulation disorders (including hereditary disorders, such as hemophilias) or to enhance coagulation and other hemostatic events in treating wounds resulting from trauma, surgery or other causes. A protein of the invention may also be useful for dissolving or inhibiting formation of thromboses and for treatment and prevention of conditions resulting therefrom (such as, for example, infarction of cardiac and central nervous system vessels (e.g., stroke).

The activity of a protein of the invention may, among other means, be measured by the following methods: Assay for hemostatic and thrombolytic activity include, without limitation, those described in: Linet et al., J. Clin. Pharmacol. 26:131-140, 1986; Burdick et al., Thrombosis Res. 45:413-419,1987; Humphrey et al., Fibrinolysis 5:71-79 (1991); Schaub, Prostaglandins 35:467-474, 1988.

Receptor/Ligand Activity

A protein of the present invention may also demonstrate activity as receptors, receptor ligands or inhibitors or agonists of receptor/ligand interactions. Examples of such receptors and ligands include, without limitation, cytokine receptors and their ligands, receptor kinases and their ligands, receptor phosphatases and their ligands, receptors involved in cell-cell interactions and their ligands (including without limitation, cellular adhesion molecules (such as selecting, integrins and their ligands) and receptor/ligand pairs involved in antigen presentation, antigen recognition and development of cellular and humoral immune responses). Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ligand interactions.

The activity of a protein of the invention may, among other means, be measured by the following methods:

Suitable assays for receptor-ligand activity include without limitation those described in:Current Protocols in Inmunology, Ed by J. E. Coligan, A. M. Kruisbeek, D. H. Margulies, E. M. Shevach, W.Strober, Pub. Greene Publishing Associates and Wiley-Interscience (Chapter 7.28, Measurement of Cellular Adhesion under static conditions 7.28.1-7.28.22), Takai et al., Proc. Natl. Acad. Sci. USA 84:6864-6868,1987; Bierer et al., J. Exp. Med. 168:1145-1156, 1988; Rosenstein et al., J. Exp. Med. 169:149-160 1989; Stoltenborg et al., J. Immunol. Methods 175:59-68, 1994; Stitt et al., Cell 80:661-670, 1995.

Anti-Inflammatory Activitv

Proteins of the present invention may also exhibit anti-inflammatory activity. The anti-inflammatory activity may be achieved by providing a stimulus to cells involved in the inflammatory response, by inhibiting or promoting cell-cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Proteins exhibiting such activities can be used to treat inflammatory conditions including chronic or acute conditions), including without limitation inflammation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of cytokines such as TNF or IL-1. Proteins of the invention may also be useful to treat anaphylaxis and hypersensitivity to an antigenic substance or material.

Cadherin/Tumor Invasion Suppressor Activity

Cadherins are calcium-dependent adhesion molecules that appear to play major roles during development, particularly in defining specific cell types. Loss or alteration of normal cadherin expression can lead to changes in cell adhesion properties linked to tumor growth and metastasis. Cadherin malfunction is also implicated in other human diseases, such as pemphigus vulgaris and pemphigus foliaceus (auto-immune blistering skin diseases), Crohn's disease, and some developmental abnormalities.

The cadherin superfamily includes well over forty members, each with a distinct pattern of expression. All members of the superfamily have in common conserved extracellular repeats (cadherin domains), but structural differences are found in other parts of the molecule. The cadherin domains bind calcium to form their tertiary structure and thus calcium is required to mediate their adhesion. Only a few amino acids in the first cadherin domain provide the basis for homophilic adhesion; modification of this recognition site can change the specificity of a cadherin so that instead of recognizing only itself, the mutant molecule can now also bind to a different cadherin. In addition, some cadherins engage in heterophilic adhesion with other cadherins.

E-cadherin, one member of the cadherin superfamily, is expressed in epithelial cell types. Pathologically, if E-cadherin expression is lost in a tumor, the malignant cells become invasive and the cancer metastasizes. Transfection of cancer cell lines with polynucleotides expressing E-cadherin has reversed cancer-associated changes by returning altered cell shapes to normal, restoring cells' adhesiveness to each other and to their substrate, decreasing the cell growth rate, and drastically reducing anchorage-independent cell growth. Thus, reintroducing E-cadherin expression reverts carcinomas to a less advanced stage. It is likely that other cadherins have the same invasion suppressor role in carcinomas derived from other tissue types. Therefore, proteins of the present invention with cadherin activity, and polynucleotides of the present invention encoding such proteins, can be used to treat cancer. Introducing such proteins or polynucleotides into cancer cells can reduce or eliminate the cancerous changes observed in these cells by providing normal cadherin expression.

Cancer cells have also been shown to express cadherins of a different tissue type than their origin, thus allowing these cells to invade and metastasize in a different tissue in the body. Proteins of the present invention with cadherin activity, and polynucleotides of the present invention encoding such proteins, can be substituted in these cells for the inappropriately expressed cadherins, restoring normal cell adhesive properties and reducing or eliminating the tendency of the cells to metastasize.

Additionally, proteins of the present invention with cadherin activity, and polynucleotides of the present invention encoding such proteins, can used to generate antibodies recognizing and binding to cadherins. Such antibodies can be used to block the adhesion of inappropriately expressed tumor-cell cadherins, preventing the cells from forming a tumor elsewhere. Such an anti-cadherin antibody can also be used as a marker for the grade, pathological type, and prognosis of a cancer, i.e. the more progressed the cancer, the less cadherin expression there will be, and this decrease in cadherin expression can be detected by the use of a cadherin-binding antibody.

Fragments of proteins of the present invention with cadherin activity, preferably a polypeptide comprising a decapeptide of the cadherin recognition site, and poly-nucleotides of the present invention encoding such protein fragments, can also be used to block cadherin function by binding to cadherins and preventing them from binding in ways that produce undesirable effects. Additionally, fragments of proteins of the present invention with cadherin activity, preferably truncated soluble cadherin fragments which have been found to be stable in the circulation of cancer patients, and polynucleotides encoding such protein fragments, can be used to disturb proper cell-cell adhesion.

Assays for cadherin adhesive and invasive suppressor activity include, without limitation, those described in: Hortsch et al. J Biol Chem 270 (32): 18809-18817, 1995; Miyaki et al. Oncogene 11: 2547-2552, 1995; Ozawa et al. Cell 63: 1033-1038, 1990.

Tumor Inhibition Activity

In addition to the activities described above for immunological treatment or prevention of tumors, a protein of the invention may exhibit other anti-tumor activities. A protein may inhibit tumor growth directly or indirectly (such as, for example, via antibody-dependent cell-mediated cytotoxicity (ADCC)). A protein may exhibit its tumor inhibitory activity by acting on tumor tissue or tumor precursor tissue, by inhibiting formation of tissues necessary to support tumor growth (such as, for example, by inhibiting angiogenesis), by causing production of other factors, agents or cell types which inhibit tumor growth, or by suppressing, eliminating or inhibiting factors, agents or cell types which promote tumor growth.

Other Activities

A protein of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhythms or caricadic cycles or rhythms; effecting the fertility of male or female subjects; effecting the metabolism, catabolism, anabolism, processing, utilization, storage or elimination of dietary fat, lipid, protein, carbohydrate, vitamins, minerals, cofactors or other nutritional factors or component(s); effecting behavioral characteristics, including, without limitation, appetite, libido, stress, cognition (including cognitive disorders), depression (including depressive disorders) and violent behaviors; providing analgesic effects or other pain reducing effects; promoting differentiation and growth of embryonic stem cells in lineages other than hematopoietic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

Administration and Dosing

A protein of the present invention (from whatever source derived, including without limitation from recombinant and non-recombinant sources) may be used in a pharmaceutical composition when combined with a pharmaceutically acceptable carrier. Such a composition may also contain (in addition to protein and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. The pharmaceutical composition of the invention may also contain cytokines, lymphokines, or other hematopoietic factors such as M-CSF, GM-CSF, TNF, IL-1, IL-2, IL-3, IL-4, IL-5, IL-6, IL-7, IL-8, IL-9, IL-10, IL-11, IL-12,IL-13, IL-14, IL-15, IFN, TNFO, TNFi, TNF2, GCSF, Meg-CSF, thrombopoietin, stem cell factor, and erythropoietin. The pharmaceutical composition may further contain other agents which either enhance the activity of the protein or compliment its activity or use in treatment. Such additional factors and/or agents may be included in the pharmaceutical composition to produce a synergistic effect with protein of the invention, or to minimize side effects. Conversely, protein of the present invention may be included in formulations of the particular cytokine, lymphokine, other hematopoietic factor, thrombolytic or anti-thrombotic factor, or anti-inflammatory agent to minimize side effects of the cytokine, lymphokine, other hematopoietic factor, thrombolytic or anti-thrombotic factor, or anti-inflammatory agent.

A protein of the present invention may be active in multimers (e.g., heterodimers or homodimers) or complexes with itself or other proteins. As a result, pharmaceutical compositions of the invention may comprise a protein of the invention in such multimeric or complexed form.

The pharmaceutical composition of the invention may be in the form of a complex of the protein(s) of present invention along with protein or peptide antigens. The protein and/or peptide antigen will deliver a stimulatory signal to both B and T lymphocytes. B lymphocytes will respond to antigen through their surface immunoglobulin receptor. T lymphocytes will respond to antigen through the T cell receptor (TCR) following presentation of the antigen by MHC proteins. MHC and structurally related proteins including those encoded by class I and class II MHC genes on host cells will serve to present the peptide antigen(s) to T lymphocytes. The antigen components could also be supplied as purified MHC-peptide complexes alone or with co-stimulatory molecules that can directly signal T cells. Alternatively antibodies able to bind surface immunolgobulin and other molecules on B cells as well as antibodies able to bind the TCR and other molecules on T cells can be combined with the pharmaceutical composition of the invention.

The pharmaceutical composition of the invention may be in the form of a liposome in which protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S.

Pat. No. 4,837,028; and U.S. Pat. No. 4,737,323, all of which are incorporated herein by reference.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

In practicing the method of treatment or use of the present invention, a therapeutically effective amount of protein of the present invention is administered to a mammal having a condition to be treated. Protein of the present invention may be administered in accordance with the method of the invention either alone or in combination with other therapies such as treatments employing cytokines, lymphokines or other hematopoietic factors. When co-administered with one or more cytokines, lymphokines or other hematopoietic factors, protein of the present invention may be administered either simultaneously with the cytokine(s), lymphokine(s), other hematopoietic factor(s), thrombolytic or anti-thrombotic factors, or sequentially. If administered sequentially, the attending physician will decide on the appropriate sequence of administering protein of the present invention in combination with cytokine(s), lymphokine(s), other hematopoietic factor(s), thrombolytic or anti-thrombotic factors.

Administration of protein of the present invention used in the pharmaceutical composition or to practice the method of the present invention can be carried out in a variety of conventional ways, such as oral ingestion, inhalation, topical application or cutaneous, subcutaneous, intraperitoneal, parenteral or intravenous injection. Intravenous administration to the patient is preferred.

When a therapeutically effective amount of protein of the present invention is administered orally, protein of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% protein of the present invention, and preferably from about 25 to 90% protein of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of protein of the present invention, and preferably from about 1 to 50% protein of the present invention.

When a therapeutically effective amount of protein of the present invention is administered by intravenous, cutaneous or subcutaneous injection, protein of the present invention will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to protein of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

The amount of protein of the present invention in the pharmaceutical composition of the present invention will depend upon the nature and severity of the condition being treated, and on the nature of prior treatments which the patient has undergone. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further. It is contemplated that the various pharmaceutical compositions used to practice the method of the present invention should contain about 0.01 μg to about 100 mg (preferably about 0.1μg to about 10 mg, more preferably about 0.1 μg to about 1 mg) of protein of the present invention per kg body weight.

The duration of intravenous therapy using the pharmaceutical composition of the present invention will vary, depending on the severity of the disease being treated and the condition and potential idiosyncratic response of each individual patient. It is contemplated that the duration of each application of the protein of the present invention will be in the range of 12 to 24 hours of continuous intravenous administration. Ultimately the attending physician will decide on the appropriate duration of intravenous therapy using the pharmaceutical composition of the present invention.

Protein of the invention may also be used to immunize animals to obtain polyclonal and monoclonal antibodies which specifically react with the protein. Such antibodies may be obtained using either the entire protein or fragments thereof as an immunogen. The peptide imnunogens additionally may contain a cysteine residue at the carboxyl terminus, and are conjugated to a hapten such as keyhole limpet hemocyanin (KLH). Methods for synthesizing such peptides are known in the art, for example, as in R.P. Merrifield, J. Amer.Chem.Soc. 85, 2149-2154 (1963); J. L. Krstenansky, et al., FEBS Lett. 211, 10 (1987). Monoclonal antibodies binding to the protein of the invention may be useful diagnostic agents for the immunodetection of the protein. Neutralizing monoclonal antibodies binding to the protein may also be useful therapeutics for both conditions associated with the protein and also in the treatment of some forms of cancer where abnormal expression of the protein is involved. In the case of cancerous cells or leukemic cells, neutralizing monoclonal antibodies against the protein may be useful in detecting and preventing the metastatic spread of the cancerous cells, which may be mediated by the protein.

For compositions of the present invention which are useful for bone, cartilage, tendon or ligament regeneration, the therapeutic method includes administering the composition topically, systematically, or locally as an implant or device. When administered, the therapeutic composition for use in this invention is, of course, in a pyrogen-free, physiologically acceptable form. Further, the composition may desirably be encapsulated or injected in a viscous form for delivery to the site of bone, cartilage or tissue damage. Topical administration may be suitable for wound healing and tissue repair. Therapeutically useful agents other than a protein of the invention which may also optionally be included in the composition as described above, may alternatively or additionally, be administered simultaneously or sequentially with the composition in the methods of the invention. Preferably for bone and/or cartilage formation, the composition would include a matrix capable of delivering the protein-containing composition to the site of bone and/or cartilage damage, providing a structure for the developing bone and cartilage and optimally capable of being resorbed into the body. Such matrices may be formed of materials presently in use for other implanted medical applications.

The choice of matrix material is based on biocompatibility, biodegradability, mechanical properties, cosmetic appearance and interface properties. The particular application of the compositions will define the appropriate formulation. Potential matrices for the compositions may be biodegradable and chemically defined calcium sulfate, tricalciumphosphate, hydroxyapatite, polylactic acid, polyglycolic acid and polyanhydrides. Other potential materials are biodegradable and biologically well-defined, such as bone or dermal collagen. Further matrices are comprised of pure proteins or extracellular matrix components. Other potential matrices are nonbiodegradable and chemically defined, such as sintered hydroxapatite, bioglass, aluminates, or other ceramics. Matrices may be comprised of combinations of any of the above mentioned types of material, such as polylactic acid and hydroxyapatite or collagen and tricalciumphosphate. The bioceramics may be altered in composition, such as in calcium-aluminate-phosphate and processing to alter pore size, particle size, particle shape, and biodegradability.

Presently preferred is a 50:50 (mole weight) copolymer of lactic acid and glycolic acid in the form of porous particles having diameters ranging from 150 to 800 microns. In some applications, it will be useful to utilize a sequestering agent, such as carboxymethyl cellulose or autologous blood clot, to prevent the protein compositions from disassociating from the matrix.

A preferred family of sequestering agents is cellulosic materials such as alkylcelluloses (including hydroxyalkylcelluloses), including methylcellulose, ethylcellulose, hydroxyethylcellulose, hydroxypropylcellulose, hydroxypropyl-methylcellulose, and carboxymethylcellulose, the most preferred being cationic salts of carboxymethylcellulose (CMC). Other preferred sequestering agents include hyaluronic acid, sodium alginate, poly(ethylene glycol), polyoxyethylene oxide, carboxyvinyl polymer and poly(vinyl alcohol). The amount of sequestering agent useful herein is 0.5-20 wt %, preferably 1-10 wt % based on total formulation weight, which represents the amount necessary to prevent desorbtion of the protein from the polymer matrix and to provide appropriate handling of the composition, yet not so much that the progenitor cells are prevented from infiltrating the matrix, thereby providing the protein the opportunity to assist the osteogenic activity of the progenitor cells.

In further compositions, proteins of the invention may be combined with other agents beneficial to the treatment of the bone and/or cartilage defect, wound, or tissue in question. These agents include various growth factors such as epidermal growth factor (EGF), platelet derived growth factor (PDGF), transforming growth factors (TGF-α and TGF-β), and insulin-like growth factor aGF).

The therapeutic compositions are also presently valuable for veterinary applications. Particularly domestic animals and thoroughbred horses, in addition to humans, are desired patients for such treatment with proteins of the present invention.

The dosage regimen of a protein-containing pharmaceutical composition to be used in tissue regeneration will be determined by the attending physician considering various factors which modify the action of the proteins, e.g., amount of tissue weight desired to be formed, the site of damage, the condition of the damaged tissue, the size of a wound, type of damaged tissue (e.g., bone), the patient's age, sex, and diet, the severity of any infection, time of administration and other clinical factors. The dosage may vary with the type of matrix used in the reconstitution and with inclusion of other proteins in the pharmaceutical composition. For example, the addition of other known growth factors, such as IGF I (insulin like growth factor I), to the final composition, may also effect the dosage. Progress can be monitored by periodic assessment of tissue/bone growth and/or repair, for example, X-rays, histomorphometric determinations and tetracycline labeling.

Polynucleotides of the present invention can also be used for gene therapy. Such polynucleotides can be introduced either in vivo or ex vivo into cells for expression in a mammalian subject. Polynucleotides of the invention may also be administered by other known methods for introduction of nucleic acid into a cell or organism (including, without limitation, in the form of viral vectors or naked DNA).

Cells may also be cultured ex vivo in the presence of proteins of the present invention in order to proliferate or to produce a desired effect on or activity in such cells.

Treated cells can then be introduced in vivo for therapeutic purposes.

Patent and literature references cited herein are incorporated by reference as if fully set forth.

What is claimed is:

1. An isolated polynucleotide selected from the group consisting of:
(a) a polynucleotide comprising the nucleotide sequence of SEQ ID NO:1;
(b) a polynucleotide comprising the nucleotide sequence of SEQ ID NO:1 from nucleotide 63 to nucleotide 1265;
(c) a polynucleotide comprising the nucleotide sequence of SEQ ID NO:1 from nucleotide 132 to nucleotide 1265;
(d) a polynucleotide comprising the nucleotide sequence of the full-length protein coding sequence of clone bd306_{—}7 deposited with the ATCC under accession number 98599;
(e) a polynucleotide encoding the full-length protein encoded by the cDNA insert of clone bd306_{—}7 deposited with the ATCC under accession number 98599;
(f) a polynucleotide comprising the nucleotide sequence of the mature protein coding sequence of clone bd306_{—}7 deposited with the ATCC under accession number 98599;
(g) a polynucleotide encoding the mature protein encoded by the cDNA insert of clone bd306_{—}7 deposited with the ATCC under accession number 98599;
(h) a polynucleotide encoding a protein comprising the amino acid sequence of SEQ ID NO:2;
(i) a polynucleotide encoding a protein comprising a fragment of the amino acid sequence of SEQ ID NO:2, the fragment comprising eight consecutive amino acids of SEQ ID NO:2; and
(j) a polynucleotide that hybridizes under stringent conditions to any one of the polynucleotides specified in (a)-(i).

2. The polynucleotide of claim 1 wherein said polynucleotide is operably linked to at least one expression control sequence.

3. A host cell transformed with the polynucleotide of claim 2.

4. The host cell of claim 3, wherein said cell is a mammalian cell.

5. A process for producing a protein encoded by the polynucleotide of claim 2, which process comprises:
(a) growing a culture of the host cell in a suitable culture medium, wherein the host cell has been transformed with the polynucleotide of claim 2; and
(b) purifying said protein from the culture.

6. A protein produced according to the process of claim 5.

7. An isolated polynucleotide encoding the protein of claim 6.

8. The polynucleotide of claim 7, wherein the polynucleotide comprises the cDNA insert of clone bd306_{—}7 deposited with the ATCC under accession number 98599.

9. A protein comprising an amino acid sequence selected from the group consisting of:
(a) the amino acid sequence of SEQ ID NO:2;
(b) the amino acid sequence of SEQ ID NO:2 from amino acid 148 to amino acid 189;
(c) fragments of the amino acid sequence of SEQ ID NO:2 comprising eight consecutive amino acids of SEQ ID NO:2; and
(d) the amino acid sequence encoded by the cDNA insert of clone bd306_{—}7 deposited with the ATCC under accession number 98599; the protein being substantially free from other mammalian proteins.

10. The protein of claim 9, wherein said protein comprises the amino acid sequence of SEQ ID NO:2.

11. The protein of claim 9, wherein said protein comprises the amino acid sequence of SEQ ID NO:2 from amino acid 148 to amino acid 189.

12. A composition comprising the protein of claim 9 and a pharmaceutically acceptable carrier.

13. An isolated polynucleotide selected from the group consisting of:
(a) a polynucleotide comprising the nucleotide sequence of SEQ ID NO:19;
(b) a polynucleotide comprising the nucleotide sequence of SEQ ID NO:19 from nucleotide 27 to nucleotide 734;
(c) a polynucleotide comprising the nucleotide sequence of SEQ ID NO:19 from nucleotide 270 to nucleotide 734;
(d) a polynucleotide comprising the nucleotide sequence of SEQ ID NO:19 from nucleotide 85 to nucleotide 1604;
(e) a polynucleotide comprising the nucleotide sequence of the full-length protein coding sequence of clone yb8_{—}1 deposited under accession number ATCC 98599;
(f) a polynucleotide encoding the full-length protein encoded by the cDNA insert of clone yb8_{—}1 deposited under accession number ATCC 98599;
(g) a polynucleotide comprising the nucleotide sequence of the mature protein coding sequence of clone yb8_{—}1 deposited under accession number ATCC 98599;
(h) a polynucleotide encoding the mature protein encoded by the cDNA insert of clone yb8_{—}1 deposited under accession number ATCC 98599;
(i) a polynucleotide encoding a protein comprising the amino acid sequence of SEQ ID NO:20;
(j) a polynucleotide encoding a protein comprising a fragment of the amino acid sequence of SEQ ID NO:20, the fragment comprising eight consecutive amino acids of SEQ ID NO:20; and
(k) a polynucleotide that hybridizes under stringent conditions to any one of the polynucleotides specified in (a)-(j).

14. A protein comprising an amino acid sequence selected from the group consisting of:
(a) the amino acid sequence of SEQ ID NO:20;
(b) the amino acid sequence of SEQ ID NO:20 from amino acid 70 to arnino acid 236;
(c) fragments of the anino acid sequence of SEQ ID NO:20 comprising eight consecutive amino acids of SEQ ID NO:20; and
(d) the amino acid sequence encoded by the cDNA insert of clone yb8_{—}1 deposited under accession number ATCC 98599; the protein being substantially free from other mammalian proteins.
